SEQUENCE LISTING (5 sequences)

SEQ ID NO: 1 - protein, 743 aa, human
  human DGKa M1 to S735 plus C-terminal Flag-Tag
MAKERGLISPSDFAQLQKYMEYSTKKVSDVLKLFEDGEMAKYVQGDAIGYEGFQQFLKIYLEVDNVPRHLSLALFQSFETGHCLNETNVTKDVVCLNDVSCYFSLLEGGRPEDKLEFTFKLYDTDRNGILDSSEVDKIILQMMRVAEYLDWDVSELRPILQEMMKEIDYDGSGSVSQAEWVRAGATTVPLLVLLGLEMTLKDDGQHMWRPKRFPRPVYCNLCESSIGLGKQGLSCNLCKYTVHDQCAMKALPCEVSTYAKSRKDIGVQSHVWVRGGCESGRCDRCQKKIRIYHSLTGLHCVWCHLEIHDDCLQAVGHECDCGLLRDHILPPSSIYPSVLASGPDRKNSKTSQKTMDDLNLSTSEALRIDPVPNTHPLLVFVNPKSGGKQGQRVLWKFQYILNPRQVFNLLKDGPEIGLRLFKDVPDSRILVCGGDGTVGWILETIDKANLPVLPPVAVLPLGTGNDLARCLRWGGGYEGQNLAKILKDLEMSKVVHMDRWSVEVIPQQTEEKSDPVPFQIINNYFSIGVDASIAHRFHIMREKYPEKFNSRMKNKLWYFEFATSESIFSTCKKLEESLTVEICGKPLDLSNLSLEGIAVLNIPSMHGGSNLWGDTRRPHGDIYGINQALGATAKVITDPDILKTCVPDLSDKRLEVVGLEGAIEMGQIYTKLKNAGRRLAKCSEITFHTTKTLPMQIDGEPWMQTPCTIKITHKNQMPMLMGPPPRSTNFFGFLSDYKDDDDK

SEQ ID NO: 2 - protein, 793 aa, human
  human DGKa M1 to S735 plus N-terminal Avi-Tag and C-terminal Flag-Tag, DGKa_hu_1Avi
MTSHHHHHHSSMGSRGIEGRGGLNDIFEAQKIEWHEGSRTSLYKKAGSATMAKERGLISPSDFAQLQKYMEYSTKKVSDVLKLFEDGEMAKYVQGDAIGYEGFQQFLKIYLEVDNVPRHLSLALFQSFETGHCLNETNVTKDVVCLNDVSCYFSLLEGGRPEDKLEFTFKLYDTDRNGILDSSEVDKIILQMMRVAEYLDWDVSELRPILQEMMKEIDYDGSGSVSQAEWVRAGATTVPLLVLLGLEMTLKDDGQHMWRPKRFPRPVYCNLCESSIGLGKQGLSCNLCKYTVHDQCAMKALPCEVSTYAKSRKDIGVQSHVWVRGGCESGRCDRCQKKIRIYHSLTGLHCVWCHLEIHDDCLQAVGHECDCGLLRDHILPPSSIYPSVLASGPDRKNSKTSQKTMDDLNLSTSEALRIDPVPNTHPLLVFVNPKSGGKQGQRVLWKFQYILNPRQVFNLLKDGPEIGLRLFKDVPDSRILVCGGDGTVGWILETIDKANLPVLPPVAVLPLGTGNDLARCLRWGGGYEGQNLAKILKDLEMSKVVHMDRWSVEVIPQQTEEKSDPVPFQIINNYFSIGVDASIAHRFHIMREKYPEKFNSRMKNKLWYFEFATSESIFSTCKKLEESLTVEICGKPLDLSNLSLEGIAVLNIPSMHGGSNLWGDTRRPHGDIYGINQALGATAKVITDPDILKTCVPDLSDKRLEVVGLEGAIEMGQIYTKLKNAGRRLAKCSEITFHTTKTLPMQIDGEPWMQTPCTIKITHKNQMPMLMGPPPRSTNFFGFLSDYKDDDDK

SEQ ID NO: 3 - protein, 8 aa, human
  SIINFEKL amino acid sequence
SIINFEKL

SEQ ID NO: 4 - protein, 8 aa, human
  Flag-Tag sequence
DYKDDDDK

SEQ ID NO: 5 - protein, 30 aa, human
  OVA-30 peptide sequence
SMLVLLPDEVSGLEQLESIINFEKLTEWTS

(19) United States
(12) Patent Application Publication
(10) Pub. No.: US 2023/0062100 A1
(43) Pub. Date: Mar. 2, 2023

(54) SUBSTITUTED AMINOQUINOLONES AS DGKALPHA INHIBITORS FOR IMMUNE ACTIVATION

(71) Applicants: Bayer Aktiengesellschaft, Leverkusen (DE); Bayer Pharma Aktiengesellschaft, Berlin (DE); DEUTSCHES KREBSFORSCHUNGSZENTRUM, Heidelberg (DE)

(72) Inventors: Norbert SCHMEES, Berlin (DE); Lars WORTMANN, Biberbach an der Riß (DE); DENNIS KIRCHOFF, Berlin (DE); Thi Thanh Uyen NGUYEN, Berlin (DE); Nicolas WERBECK, Berlin (DE); Ulf BÖMER, Glienicke (DE); Kirstin PETERSEN, Berlin (DE); Christina KOBER, Lollar (DE); Detlef STÖCKIGT, Potsdam (DE); Christian LECHNER, Berlin (DE); Robin Michael MEIER, Wain (DE); Simon Anthony HERBERT, Berlin (DE); Isabel Patrizia KERSCHGENS, Zürich (CH); DIRK KOSEMUND, Berlin (DE); Julien LEFRANC, Darmstadt (DE); Rienk OFFRINGA, Heidelberg (DE)

(73) Assignees: Bayer Aktiengesellschaft, Leverkusen (DE); Bayer Pharma Aktiengesellschaft, Berlin (DE); DEUTSCHES KREBSFORSCHUNGSZENTRUM, Heidelberg (DE)

(21) Appl. No.: 17/780,903

(22) PCT Filed: Nov. 24, 2020
(86) PCT No.: PCT/EP2020/083196

§ 371 Date: May 27, 2022

(30) Foreign Application Priority Data
Nov. 28, 2019 (EP) 19212243.0

(51) Int. Cl. A61K 31/4709; C07D 401/04; C07D 401/14; C07D 413/14; C07D 405/14; C07F 9/6558; C07D 417/14; A61K 31/513; A61K 31/496; A61K 31/506; A61K 31/675; A61K 31/55; A61P 37/04; A61P 35/00; A61K 45/06

(52) U.S. Cl. CPC A61K 31/4709; C07D 401/04; C07D 401/14; C07D 413/14; C07D 405/14; C07F 9/65583; C07D 417/14; A61K 31/513; A61K 31/496; A61K 31/506; A61K 31/675; A61K 31/55; A61P 37/04; A61P 35/00; A61K 45/06

(57) ABSTRACT

The present invention covers aminoquinolone compounds of general formula (I): in which R¹, R², R³, R⁴, R⁵, R⁶, R⁷, R⁸, X and n are as defined herein, methods of preparing said compounds, intermediate compounds useful for preparing said compounds, pharmaceutical compositions and combinations comprising said compounds and the use of said compounds for manufacturing pharmaceutical compositions for the treatment and/or prophylaxis of diseases, in particular of diacylglycerol kinase alpha regulated disorders, as a sole agent or in combination with other active ingredients.

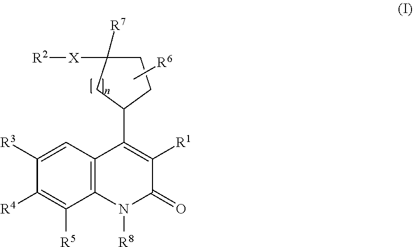

The present invention covers substituted aminoquinolone compounds of general formula (I) as described and defined herein, methods of preparing said compounds, intermediate compounds useful for preparing said compounds, pharmaceutical compositions and combinations comprising said compounds, and the use of said compounds for manufacturing pharmaceutical compositions for the treatment or prophylaxis of diseases, in particular of diacylglycerol kinase alpha (DGKalpha, DGKα) regulated disorders, as a sole agent or in combination with other active ingredients.

The compounds of general formula (I) inhibit DGKα and enhance T cell mediated immune response. This is a new strategy to use the patient's own immune system to overcome immunoevasive strategies utilized by many neoplastic disorders, respectively cancer and by this enhancing anti-tumor immunity. Furthermore, said compounds are used in particular to treat disorders such as viral infections or conditions with dysregulated immune responses or other disorders associated with aberrant DGKα signaling.

The present invention further relates to the use, respectively to the use of the compounds of general formula (I) for manufacturing pharmaceutical compositions for enhancement of T cell mediated immune response.

The present invention further relates to the use, respectively to the use of the compounds of general formula (I) for manufacturing pharmaceutical compositions for the treatment of cancer.

The present invention further relates to the use, respectively to the use of the compounds of general formula (I) for manufacturing pharmaceutical compositions for the treatment or prophylaxis of fibrotic disorders, virus infections, cardiac diseases and lymphoproliferative disorders.

### BACKGROUND

Diacylglycerol kinases (DGKs) represent a family of enzymes that catalyze phosphorylation of the membrane lipid sn-1,2 diacylglycerol (DAG) to form phosphatidic acid (PA) (Eichmann and Lass, Cell Mol Life Sci. 2015; 72: 3931). In T cells, DAG is formed downstream of the T cell receptor (TCR) after activation of the gamma 1 isoform of phospholipase C (PLCy1) and cleavage of phosphatidylinositol 4,5-biphosphate (PIP2) into DAG and an additional second messenger, inositol 1,4,5-triphosphate (IP3) (Krishna and Zhong, Front. Immunol 2013, 4, 178). Whereas, IP3 is important in facilitating release of calcium from the endoplasmic reticulum, DAG interacts with other proteins important in TCR signal transduction, such as Protein kinase Cθ (Quann et al., Nat Immunol 2011(7), 647) and the Ras activating protein RasGRP1 (Krishna and Zhong, Front. Immunol 2013, 4:178). Although, three isoforms of DGK are known to be present within T cells [DGKα (DGKalpha), DGKδ (DGKdelta), and DGKζ (DGKzeta)], only two, DGKα and DGKζ are thought to play an important role in facilitating DAG metabolism downstream of the TCR (Joshi and Koretzky, Int. J. Mol. Sci. 2013, 14, 6649).

Targeting the activity of DGKα in T cells, either by germline deletion, or with chemical inhibitors, results in enhanced and sustained signaling downstream of T cells, as assessed by prolonged phosphorylation of downstream molecules, such as extracellular signal-related kinases 1/2 (ERK1/2 (Zhong et al., Nat Immunol 2003, 4, 882; Olenchock et al., Nat Immunol 2006, 7, 1174; Riese et al., J. Biol. Chem 2011, 286, 5254). Furthermore, the overexpression of DGKα induces a state of decreased functional activity resembling an anergy-like state (Zha et al., Nat Immunol 2006, 7, 1166). In contrast, deletion of DGKα in T cells with enhanced production of effector cytokines, such as IL2 and IFNγ, and enhanced proliferation (Zhong et al., Nat Immunol 2003, 4, 882 Olenchock et al., Nat Immunol 2006, 7, 1174).

These findings suggest that DGKα might serve as a useful target for enhancing T cell anti-tumor activity. The role of DGKα in anti-tumor responses was studied recently in human tumor-infiltrating CD8+T cells (CD8-TILs) from patients with renal cell carcinoma (RCC) (Prinz et al., J. Immunol 2012, 188, 5990). CD8-TILs from RCCs were defective in lytic granule exocytosis and their ability to kill target cells. While proximal signaling events were intact in response to TCR engagement, CD8-TILs exhibited decreased phosphorylation of ERK when compared to non-tumor-infiltrating CD8+T cells. Treatment of CD8-TILs with an inhibitor of DGKα activity rescued killing ability of target cells, increased basal levels of phosphorylation of ERK, and increased PMA/ionomycin-stimulated phosphorylation of ERK.

In addition, Arranz-Nicolas et al show that DGK inhibitors promoted not only Ras/ERK signaling but also AP-1 (Activator protein-1) transcription, facilitated DGKα membrane localization, reduced the requirement for costimulation, and cooperated with enhanced activation following DGKζ silencing/deletion. In contrast with enhanced activation triggered by pharmacological inhibition, DGKα silencing/genetic deletion led to impaired Lck (lymphocyte-specific protein tyrosine kinase) activation and limited costimulation responses. (Arranz-Nicolas et al., Canc Immun, Immunother 2018, 67(6), 965).

In addition, abtigen-specific CD8⁺ T cells from DGKα^{-/-} and DGKζ^{-/-} mice show enhanced expansion and increased cytokine production following (Lymphocytic choriomeningitis virus) infection (Shin et al. J. Immunol, 2012).

Additionally, the adoptive transfer of CAR (chimeric antigen receptor)-T cells deficient in DGKα demonstrated increased efficacy compared to wild type CAR T cells T cells in the treatment of murine mesothelioma (Riese et al., Cancer Res 2013, 73(12), 3566) and a glioblastoma xenograft mouse model (Jung et al. Cancer Res. 2018, 78(16), 4692).

Apart from T-cell regulation, DGKα also plays a role in cancer, mediating numerous aspects of cancer cell progression including survival (Bacchiocchi et al., Blood, 2005, 106(6), 2175; Yanagisawa et al. Biochim Biophys Acta 2007, 1771, 462), migration and invasion of cancer cells (Baldanzi et al., Oncogene 2008, 27, 942; Filigheddu et al., Anticancer Res 2007, 27, 1489; Rainero et al., J Cell Biol 2012, 196(2): 277). In particular, it has been reported that DGKα is over expressed in hepatocellular carcinoma (Takeishi et al., J Hepatol 2012, 57, 77) and melanoma cells (Yanagisawa et al., Biochim Biophys Acta 2007, 1771, 462) while other reports suggested that the growth of colon and breast cancer cell lines was significantly inhibited by DGKα-siRNA16 and DGKα/atypical PKC/b1 integrin signalling pathway was crucial for matrix invasion of breast carcinoma cells (Rainero et al., PLoS One 2014, 9(6): e97144) In addition, expression is also higher in lymphonodal metastasis than in breast original tumour (Hao et al., Cancer 2004, 100, 1110).

Additionally, a study testing the importance of DGKα in glioblastoma multiforme (GBM) cells found that concurrent administration of the relatively non-specific DGKα inhibitor R59022 resulted in decreased growth of intracranially injected GBM tumors. (Dominguez et al. Cancer Discov 2013, 3(7): 782).

Also, DGKα promotes esophageal squamous cell carcinoma (ESCC) progression, supporting DGKα as a potential target for ESCC therapy (Chen et al., Oncogene, 2019, 38 (14) 2533).

In addition, pharmacological inhibition of DGK diminished both airway inflammation and airway hyperresponsiveness in mice and also reduced bronchoconstriction of human airway samples in vitro by blocking T helper 2 (T_{H}2) differentiation (Singh et al., Sci Signal. 2019, 12, eaax3332).

Furthermore, inhibition of DGKα has the potential to reverse the life-threatening Epstein-Barr virus (EBV)-associated immunopathology that occurs in patients X-linked lymphoproliferative disease (XLP-1) patients (Ruffo et al., Sci Transl Med. 2016, 13, 8, 321; Velnati et al., Eur J Med Chem. 2019, 164,378).

In addition, DGKα exacerbates cardiac injury after ischemia/reperfusioncardiac diseases (Sasaki et al., Heart Vessels, 2014, 29, 110).

Taken together, the findings from these studies argue that restraining DGKα activity in T cells and tumor cells may prove valuable in generating more vigorous immune responses against pathogens and tumors and in amoiroting Th2 driven (ato) immune deseases (in re-balancing the immune-systeme). In addition, inhibiting DGKα activity has a therapeutic potential in targeting tumors directly as well as addressing fibrotic disorders, virus infection associated pathologies, cardiac diseases and lymphoproliferative disorders.

### PRIOR ART

DGKα inhitors were reported in the literature. R59022 (A) was identified to act on DGKα in red blood cells (de Chaffoy de Courcelles et. al., J. Biol. Chem. Vol 260, No. 29, (1985), p15762-70). Structurally related R59949 (B) was identified to act on DGKα in T-lymphocytes by inhibiting the transformation of 1,2-diacylglycerols to their respective phosphatidic acids (Jones et. al., J. Biol. Chem. Vol 274, No. 24, (1999), p16846-52). Ritanserin (C), originally identified as a serotonine receptor antagonist, showed comparable activity on DGKα such as the two R cpds (A) and (B) (Boroda et. al., BioChem. Pharm. 123, (2017), 29-39).

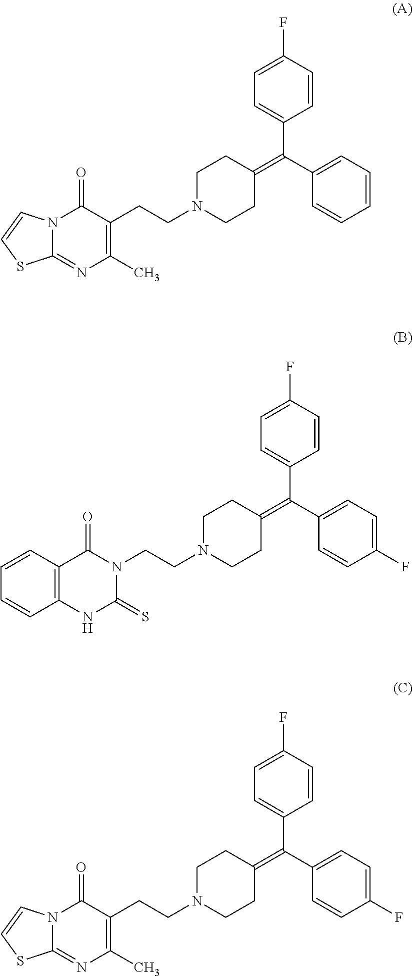 [SMILES: Cc1nc2sccn2c(=O)c1CCN1CCC(=C(c2ccccc2)c2ccc(F)cc2)CC1 ; O=c1c2ccccc2[nH]c(=S)n1CCN1CCC(=C(c2ccc(F)cc2)c2ccc(F)cc2)CC1 ; Cc1nc2sccn2c(=O)c1CCN1CCC(=C(c2ccc(F)cc2)c2ccc(F)cc2)CC1]

A further structure, CU-3 (D) was identified as a first compound with sub-micromolar inhibitory activity on DGKα (Sakane et. al., J. Lipid Res. Vol 57, (2016), p368-79).

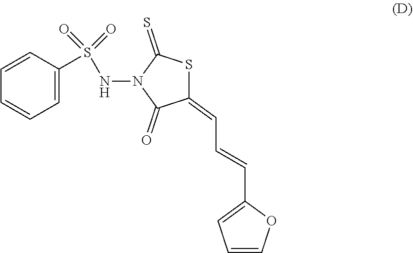 [SMILES: O=C1/C(=C\C=C\c2ccco2)SC(=S)N1NS(=O)(=O)c1ccccc1]

AMB639752 (E) was describe as a further DGKα selective inhibitor with micromolar activity (S. Velnati et al. Eur J. Med. Chem 2019, 164, p378-390.).

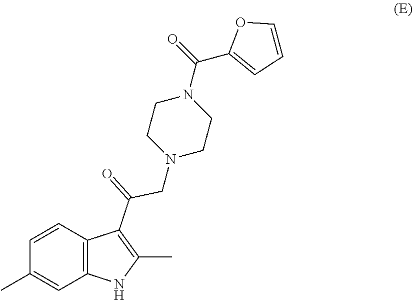 [SMILES: Cc1ccc2c(C(=O)CN3CCN(C(=O)c4ccco4)CC3)c(C)[nH]c2c1]

WO2020/151636 relates to azaquinolinones as PDE9 inhibitor compounds for treatment of PDE9 mediated diseases.

WO2020/143626 relates to quinolinones as PDE9 inhibitor compounds for treatment of PDE9 mediated diseases.

WO2020/182076 relates to the use of phosphodiesterase inhibitor compounds in the preparation of drugs for treating heart failure in mammals.

WO2020/006016 and WO2020/006018 describe Naphthydrinone compounds as T cell activators, which inhibt the activity of DGKα and/or DGK, for treatment of viral infections and proliferative disorders, such as cancer.

WO2017/019723 A1 relates to azacyanoquinolinone compounds which may be useful as therapeutic agents for the treatment of central nervous system disorders associated with phosphodiesterase 9 (PDE9). It also relates to the use of the compounds compounds for treating neurological and psychiatric disorders.

WO2004/074218 describes MIF-inhibitors and multiple uses thereof, among others for treatment of cancer.

WO2007/109251 describes the use of TNFα inhibitors for treatment of diseases, among others for treatment of cancer.

WO 2012/142498 and WO2012/009649 describe MIF-inhibitors and multiple uses thereof, among others in cancer therapy. These patent applications claim an extremely high number of compounds. However, many of these theoretical compounds are not specifically disclosed.

However, the state of the art does not describe:
the specific substituted aminoquinolone compounds of general formula (I) of the present invention as described and defined herein, i.e. compounds having a 2-oxo-1,2-dihydroquinoline core bearing:
   in its 1-position a methyl- or an ethyl group,
   in its 3-position a cyano-, carbamoyl-, alkylcarbamoyl-, dialkylcarbamoyl- or alkoxycarbonyl group,
   in its 4-postion a pyrolidinyl-, piperidinyl- or azepanyl group; and
   as a substituent of said pyrolidinyl-, piperidinyl- or azepanyl group a —X-phenyl, —X-naphthyl or —X-(5- to 10-membered heteroaryl) group,
or stereoisomers, tautomers, N-oxides, hydrates, solvates, salts thereof, or mixtures of same, as described and defined herein, and as hereinafter referred to as “compounds of general formula (I)” or “compounds of the present invention”,
or their pharmacological activity.

It is desirable to provide novel compounds having prophylactic and therapeutic properties.

Accordingly, it is an object of the present invention to provide compounds and pharmaceutical compositions comprising these compounds used for prophylactic and therapeutic use in DGKα regulated disorders in a T cell immune-stimulatory or immune-modifing manner. DGKα regulated disorders comprise conditions with dysregulated immune responses, particularly in an immunologically suppressed tumor microenvironment in cancer, autoimmune diseases, viral infections as well as other disorders associated with aberrant DGKα signalling, e.g. fibrotic diseases. Said compounds can be used as sole agent or in combination with other active ingredients.

It has now been found, and this constitutes the basis of the present invention, that the compounds of the present invention have surprising and advantageous properties.

In particular, the compounds of the present invention have surprisingly been found to effectively inhibit the DGKα protein and enhance T-cell mediated immunity. Accordingly, they provide novel structures for the therapy of human and animal disorders, in particular of cancers, and may therefore be used for the treatment or prophylaxis of hyperproliferative disorders, such as cancer, for example.

### DESCRIPTION OF THE INVENTION

In accordance with a first aspect, the present invention covers compounds of general formula (I):

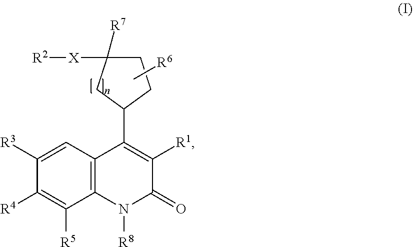

in which
n represents an integer selected from 1, 2 and 3,
X represents a group selected from
   —O—, —S—, —S(═O)—, —S(═O)₂—, *-(C(R¹⁶)(R¹⁷))ₒ-#, *-O—C(R¹⁶)₂—(C(R¹⁶)(R¹⁷))ₚ-#, *-(C(R¹⁶)(R¹⁷))ₚ—C(R¹⁶)₂—O-#,

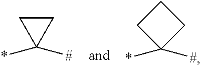

wherein * indicates the point of attachment to R² and #indicates the point of attachment to the pyrrolidine-, piperidine- and azepane moiety,
R¹ represents a group selected from cyano, —C(═O)NH₂, —C(═O)N(H)CH₃, —C(═O)N(H)C₂H₅, —C(═O)N(CH₃)₂ and —C(═O)OR¹⁵,
R² represents a group selected from phenyl, naphthyl and 5- to 10-membered heteroaryl,
   which 5- to 10-membered heteroaryl group is connected to the rest of the molecule via a carbon atom of said 5- to 10-membered heteroaryl group,
   and
   which phenyl, naphthyl and 5- to 10-membered heteroaryl group is optionally substituted, one, two, three or four times, each substituent independently selected from a halogen atom or a group selected from
   C₁-C₆-alkyl, C₃-C₆-cycloalkyl, C₁-C₆-hydroxyalkyl, C₁-C₆-haloalkyl, (C₁-C₂-alkoxy)-(C₁-C₆-alkyl)-, C₁-C₆-alkoxy, (C₁-C₂-alkoxy)-(C₁-C₆-alkoxy)-, C₁-C₆-haloalkoxy, C₃-C₆-cycloalkyloxy, phenoxy, —SR¹⁴, —S(═O)R¹⁴, —S(═O)₂R¹⁴, —P(═O)(R¹⁴)₂, nitro, cyano, hydroxy, —N(R⁹)(R¹⁰), —C(═O)N(R⁹)(R¹⁰), —C(═O)R¹¹, —C(═O)₂R¹⁵, —N(R¹²)C(═O)R¹³, —N(R¹²)S(═O)₂R¹⁴, —N═S(═NH)(R¹⁴)₂, —N═S(═O)(R¹⁴)₂, —S(═O)(═NR¹⁴)R¹³, 4- to 7-membered heterocycloalkyl, 5- to 7-membered heterocycloalkenyl, (4- to 7-membered heterocycloalkyl)oxy, phenyl and 5- or 6-membered heteroaryl,
   or two substituents of said phenyl group, when they are attached to adjacent ring atoms, are optionally linked to one another in such a way that they jointly form a group selected from
   —(CH₂)₃—, —(CH₂)₄—, —O—(CH₂)₂—, —(CH₂)₂—O—, —CH₂—O—CH₂—, —O—(CH₂)₃—, —(CH₂)₃—O—, —CH₂—O—(CH₂)₂—, —(CH₂)₂O—CH₂—, —O—CH₂—O— and —O—(CH₂)₂—O—,
      wherein said 4- to 7-membered heterocycloalkyl group
      and 5- to 7-membered heterocycloalkenyl group is connected to the rest of the molecule via a carbon atom of said
      4- to 7-membered heterocycloalkyl group and
      5- to 7-membered heterocycloalkenyl group,
      and
      wherein said 4- to 7-membered heterocycloalkyl group,
      5- to 7-membered heterocycloalkenyl group and
      (4- to 7-membered heterocycloalkyl)oxy group is optionally substituted, one, two or three times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl, —N(R⁹)(R¹⁰) and oxo,
      and
      wherein said C₁-C₆-alkyl and C₁-C₆-alkoxy group is optionally substituted with a group selected from
      C₃-C₄-cycloalkyl, phenyl and 4- to 7-membered heterocycloalkyl,
         wherein said 4- to 7-membered heterocycloalkyl group is connected to the rest of the molecule via a carbon atom of said 4- to 7-membered heterocycloalkyl group and
         wherein said 4- to 7-membered heterocycloalkyl group is optionally substituted, one, two or three times, each substituent independently selected from a halogen atom or a group selected from
         C₁-C₂-alkyl, C₁-C₂-haloalkyl, cyano, hydroxy, C₁-C₂-alkoxy, C₃-C₄-cycloalkyl, —N(R⁹)(R¹⁰) and oxo,
         and
         which phenyl group is optionally substituted, one or two times, each substituent independently selected from a halogen atom or a group selected from
         C₁-C₂-alkyl, C₁-C₂-haloalkyl, cyano, hydroxy, C₁-C₂-alkoxy, C₃-C₄-cycloalkyl and —N(R⁹)(R¹⁰),
         and
         which C₃-C₄-cycloalkyl group is optionally substituted, one or two times, each substituent independently selected from a halogen atom or a group selected from
         cyano and hydroxy,
      and
      wherein said C₃-C₆-cycloalkyl group is optionally substituted, one or two times, each substituent independently selected from a halogen atom or a C₁-C₄-alkyl group,
      and
      wherein said phenyl, phenoxy and 5- or 6-membered heteroaryl group is optionally substituted, one or two times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl and —N(R⁹)(R¹⁰)
R³ represents a hydrogen atom or a halogen atom or a group selected from
   C₁-C₆-alkyl, C₂-C₆-alkenyl, C₂-C₆-alkynyl, C₃-C₆-cycloalkyl, C₄-C₆-cycloalkenyl, C₁-C₆-hydroxyalkyl, C₁-C₆-haloalkyl, (C₁-C₂-alkoxy)-(C₁-C₆-alkyl)-, C₁-C₆-alkoxy, (C₁-C₂-alkoxy)-(C₁-C₆-alkoxy)-, C₁-C₄-haloalkoxy, C₃-C₆-cycloalkyloxy, phenoxy, —SR¹⁴, —S(═O)R¹⁴, —S(═O)₂R¹⁴, cyano, hydroxy, —N(R⁹)(R¹⁰), —C(═O)N(R⁹)(R¹⁰), —C(═O)R¹, —N(R¹²)C(═O)R¹³, —N(R¹²)S(═O)₂R¹⁴, —N═S(═NH)(R¹⁴)₂, —N═S(═O)(R¹⁴)₂, —P(═O)(R¹⁴)₂, 4- to 7-membered heterocycloalkyl, 5- to 7-membered heterocycloalkenyl, (4- to 7-membered heterocycloalkyl)oxy, phenyl and 5- or 6-membered heteroaryl,
      wherein said 4- to 7-membered heterocycloalkyl group and 5- to 7-membered heterocycloalkenyl group is connected to the rest of the molecule via a carbon atom of said 4- to 7-membered heterocycloalkyl group and 5- to 7-membered heterocycloalkenyl group,
      and
      wherein said 4- to 7-membered heterocycloalkyl group, 5- to 7-membered heterocycloalkenyl group and (4- to 7-membered heterocycloalkyl)oxy group is optionally substituted, one, two or three times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl, —N(R⁹)(R¹⁰) and oxo,
      and
      wherein said C₁-C₆-alkyl, C₂-C₆-alkenyl, C₂-C₆-alkynyl and C₁-C₆-alkoxy group is optionally substituted with a group selected from
      C₃-C₄-cycloalkyl, phenyl and 4- to 7-membered heterocycloalkyl,
         wherein said 4- to 7-membered heterocycloalkyl group is connected to the rest of the molecule via a carbon atom of said 4- to 7-membered heterocycloalkyl group,
         and
         wherein said 4- to 7-membered heterocycloalkyl group is optionally substituted, one, two or three times, each substituent independently selected from a halogen atom or a group selected from
         C₁-C₂-alkyl, C₁-C₂-haloalkyl, cyano, hydroxy, C₁-C₂-alkoxy, C₃-C₄-cycloalkyl, —N(R⁹)(R¹⁰) and oxo,
         and
         which phenyl group is optionally substituted, one or two times, each substituent independently selected from a halogen atom or a group selected from
         C₁-C₂-alkyl, C₁-C₂-haloalkyl, cyano, hydroxy, C₁-C₂-alkoxy, C₃-C₄-cycloalkyl and —N(R⁹)(R¹⁰),
         and
         which C₃-C₄-cycloalkyl group is optionally substituted, one or two times, each substituent independently selected from a halogen atom or a group selected from
         cyano and hydroxy,
      and
      wherein said C₃-C₆-cycloalkyl and C₄-C₆-cycloalkenyl group is optionally substituted, one or two times, each substituent independently selected from a halogen atom or a C₁-C₄-alkyl group,
      and
      wherein said phenyl, phenoxy and 5- or 6-membered heteroaryl group is optionally substituted, one or two times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl and —N(R⁹)(R¹⁰),
R⁴ represents a hydrogen atom or a halogen atom or a group selected from
   C₁-C₆-alkyl, C₂-C₆-alkenyl, C₂-C₆-alkynyl, C₃-C₆-cycloalkyl, C₄-C₆-cycloalkenyl, C₁-C₆-hydroxyalkyl, C₁-C₆-haloalkyl, (C₁-C₂-alkoxy)-(C₁-C₆-alkyl)-, C₁-C₆-alkoxy, (C₁-C₂-alkoxy)-(C₁-C₆-alkoxy)-, C₁-C₄-haloalkoxy, C₃-C₆-cycloalkyloxy, —S(═O)R¹⁴, —S(═O)₂R¹⁴, cyano, hydroxy, —N(R⁹)(R¹⁰), —N(R¹⁸)(R¹⁹), —C(═O)N(R⁹)(R¹⁰), —C(═O)R¹, —N(R¹²)C(═O)R¹³, —N(R¹²)S(═O)₂R¹⁴, —N═S(═NH)(R¹⁴)₂, —N═S(═O)(R¹⁴)₂, —P(═O)(R¹⁴)₂, 4- to 7-membered heterocycloalkyl, 5- to 7-membered heterocycloalkenyl, (4- to 7-membered heterocycloalkyl)oxy, phenyl and 5- or 6-membered heteroaryl,
      wherein said 4- to 7-membered heterocycloalkyl group and 5- to 7-membered heterocycloalkenyl group is connected to the rest of the molecule via a carbon atom of said 4- to 7-membered heterocycloalkyl group and 5- to 7-membered heterocycloalkenyl group,
      and
      wherein said 4- to 7-membered heterocycloalkyl group, 5- to 7-membered heterocycloalkenyl group and (4- to 7-membered heterocycloalkyl)oxy group is optionally substituted, one, two or three times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl, —N(R⁹)(R¹⁰) and oxo,
      and
      wherein said C₁-C₆-alkyl, C₂-C₆-alkenyl, C₂-C₆-alkynyl and C₁-C₆-alkoxy group is optionally substituted with a group selected from
      C₃-C₄-cycloalkyl, phenyl and 4- to 7-membered heterocycloalkyl,
         wherein said 4- to 7-membered heterocycloalkyl group is connected to the rest of the molecule via a carbon atom of said 4- to 7-membered heterocycloalkyl group,
         and
         wherein said 4- to 7-membered heterocycloalkyl group is optionally substituted, one, two or three times, each substituent independently selected from a halogen atom or a group selected from
         C₁-C₂-alkyl, C₁-C₂-haloalkyl, cyano, hydroxy, C₁-C₂-alkoxy, C₃-C₄-cycloalkyl, —N(R⁹)(R¹⁰) and oxo,
         and
         which phenyl group is optionally substituted, one or two times, each substituent independently selected from a halogen atom or a group selected from
         C₁-C₂-alkyl, C₁-C₂-haloalkyl, cyano, hydroxy, C₁-C₂-alkoxy, C₃-C₄-cycloalkyl and —N(R⁹)(R¹⁰),
         and
         which C₃-C₄-cycloalkyl group is optionally substituted, one or two times, each substituent independently selected from a halogen atom or a group selected from
         cyano and hydroxy,
      and
      wherein said C₃-C₆-cycloalkyl and C₄-C₆-cycloalkenyl group is optionally substituted, one or two times, each substituent independently selected from a halogen atom or a C₁-C₄-alkyl group,
      and
      wherein said phenyl and 5- or 6-membered heteroaryl group is optionally substituted, one or two times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl and —N(R⁹)(R¹⁰),
R⁵ represents a hydrogen atom or a halogen atom or a group selected from
   C₁-C₆-alkyl, C₂-C₆-alkenyl, C₂-C₆-alkynyl, C₃-C₆-cycloalkyl, C₄-C₆-cycloalkenyl, C₁-C₆-hydroxyalkyl, C₁-C₆-haloalkyl, (C₁-C₂-alkoxy)-(C₁-C₆-alkyl)-, C₁-C₆-alkoxy, (C₁-C₂-alkoxy)-(C₁-C₆-alkoxy), —C₁-C₄-haloalkoxy, C₃-C₆-cycloalkyloxy, phenoxy, —SR¹⁴, —S(═O)R¹⁴, S(═O)₂R¹⁴, cyano, hydroxy, —N(R⁹)(R¹⁰), —C(═O)N(R⁹)(R¹⁰), —C(═O)R¹¹, —N(R¹²)C(═O)R¹³, —N(R¹²)S(═O)₂R¹⁴, —N═S(═NH)(R¹⁴)₂, —N═S(═O)(R¹⁴)₂, —P(═O)(R¹⁴)₂, 4- to 7-membered heterocycloalkyl, 5- to 7-membered heterocycloalkenyl, (4- to 7-membered heterocycloalkyl)oxy, phenyl and 5- or 6-membered heteroaryl,
      wherein said 4- to 7-membered heterocycloalkyl group and 5- to 7-membered heterocycloalkenyl group is connected to the rest of the molecule via a carbon atom of said 4- to 7-membered heterocycloalkyl group and 5- to 7-membered heterocycloalkenyl group,
      and
      wherein said 4- to 7-membered heterocycloalkyl group, 5- to 7-membered heterocycloalkenyl group and (4- to 7-membered heterocycloalkyl)oxy group is optionally substituted, one, two or three times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl, —N(R⁹)(R¹⁰) and oxo,
      and
      wherein said C₁-C₆-alkyl, C₂-C₆-alkenyl, C₂-C₆-alkynyl and C₁-C₆-alkoxy group is optionally substituted with a group selected from
      C₃-C₄-cycloalkyl, phenyl and 4- to 7-membered heterocycloalkyl,
         wherein said 4- to 7-membered heterocycloalkyl group is connected to the rest of the molecule via a carbon atom of said 4- to 7-membered heterocycloalkyl group,
         and
         wherein said 4- to 7-membered heterocycloalkyl group is optionally substituted, one, two or three times, each substituent independently selected from a halogen atom or a group selected from
         C₁-C₂-alkyl, C₁-C₂-haloalkyl, cyano, hydroxy, C₁-C₂-alkoxy, C₃-C₄-cycloalkyl, —N(R⁹)(R¹⁰) and oxo,
         and
         which phenyl group is optionally substituted, one or two times, each substituent independently selected from a halogen atom or a group selected from
         C₁-C₂-alkyl, C₁-C₂-haloalkyl, cyano, hydroxy, C₁-C₂-alkoxy, C₃-C₄-cycloalkyl and —N(R⁹)(R¹⁰),
         and
         which C₃-C₄-cycloalkyl group is optionally substituted, one or two times, each substituent independently selected from a halogen atom or a group selected from
         cyano and hydroxy,
      and
      wherein said C₃-C₆-cycloalkyl and C₄-C₆-cycloalkenyl group is optionally substituted, one or two times, each substituent independently selected from a halogen atom or a C₁-C₄-alkyl group,
      and
      wherein said phenyl, phenoxy and 5- or 6-membered heteroaryl group is optionally substituted, one or two times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl and —N(R⁹)(R¹⁰),
R⁶ represents a hydrogen atom, or a fluorine atom or a group selected from
   C₁-C₄-alkyl, C₁-C₄-hydroxyalkyl, C₁-C₄-alkoxy, hydroxy and oxo,
R⁷ represents a hydrogen atom or a halogen atom or a group selected from
   C₁-C₄-alkyl, C₁-C₄-alkoxy, hydroxy and cyano,
R³ represents a group selected from methyl and ethyl,
R⁹ and R¹⁰ represent, independently from each occurrence, a hydrogen atom or a group selected from
   C₁-C₄-alkyl, (C₁-C₄-alkoxy)-(C₂-C₄-alkyl)-, C₃-C₄-cycloalkyl and C₂-C₄-haloalkyl,
   or
R⁹ and R¹⁰ together with the nitrogen to which they are attached represent a nitrogen containing 4- to 7-membered heterocycloalkyl group or 5- to 7-membered heterocycloalkenyl group,
   wherein said nitrogen containing 4- to 7-membered heterocycloalkyl group and 5- to 7-membered heterocycloalkenyl group are optionally substituted, one, two or three times, each substituent independently selected from a halogen atom or a group selected from
   C₁-C₄-alkyl, C₃-C₄-cycloalkyl, hydroxy and oxo,
   or
   two substituents, which are attached to the same carbon atom of said nitrogen containing 4- to 7-membered heterocycloalkyl group, together with the carbon atom to which they are attached, represent a 4- to 7-membered heterocycloalkyl group,
      wherein said 4- to 7-membered heterocycloalkyl group is optionally substituted, one or two times, each substituent independently selected from a halogen atom or a group selected from
      C₁-C₄-alkyl, C₃-C₄-cycloalkyl, C₁-C₄-haloalkyl, hydroxy and oxo,
R¹¹ represents a hydrogen atom or group selected from
   C₁-C₄-alkyl, C₁-C₄-hydroxyalkyl, C₁-C₄-haloalkyl, phenyl and 5- or 6-membered heteroaryl,
      wherein said phenyl group and 5- or 6-membered heteroaryl group is optionally substituted, one or two times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl and —N(R⁹)(R¹⁰),
R¹² represents a hydrogen atom or a C₁-C₄-alkyl group,
R¹³ represents a hydrogen atom or a group selected from
   C₁-C₆-alkyl, phenyl and 5- or 6-membered heteroaryl,
      wherein said phenyl group and 5- or 6-membered heteroaryl group is optionally substituted, one or two times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl and —N(R⁹)(R¹⁰)
R¹⁴ represents a group selected from C₁-C₆-alkyl, C₁-C₆-haloalkyl, C₃-C₆-cycloalkyl, phenyl and 5- or 6-membered heteroaryl,
   wherein said phenyl group and 5- or 6-membered heteroaryl group is optionally substituted, one or two times, each substituent independently selected from a halogen atom or a group selected from
   C₁-C₂-alkyl, C₁-C₂-haloalkyl, cyano, hydroxy, C₁-C₂-alkoxy, C₃-C₄-cycloalkyl and —N(R⁹)(R¹⁰),
R¹⁵ represents a hydrogen atom or a C₁-C₄-alkyl group,
R¹⁶ represent, indepently from each other, a hydrogen atom or a halogen atom or a group selected from
   C₁-C₂-alkyl and C₁-C₂-haloalkyl,
R¹⁷ represent, indepently from each other, a hydrogen atom or a halogen atom or a group selected from
   C₁-C₂-alkyl, C₁-C₂-haloalkyl, C₁-C₂-alkoxy, C₁-C₂-haloalkoxy and hydroxy,
R¹⁸ represents a hydrogen atom or a group selected from
   C₁-C₄-alkyl, C₃-C₄-cycloalkyl and C₂-C₄-haloalkyl,
R¹⁹ represents a 4- to 7-membered heterocycloalkyl group,
   wherein said 4- to 7-membered heterocycloalkyl group is optionally substituted, one, two or three times, each substituent independently selected from a halogen atom or a group selected from
   C₁-C₄-alkyl, C₃-C₄-cycloalkyl, C₁-C₄-alkoxy, hydroxy and oxo,
   and
   wherein said 4- to 7-membered heterocycloalkyl group is connected to the rest of the molecule via a carbon atom of the 4- to 7-membered heterocycloalkyl group,
o represents an integer selected from 1 or 2,
and
p represents an integer selected from 0 and 1,
and stereoisomers, tautomers, N-oxides, hydrates, solvates, and salts thereof, and mixtures of same.

### Definitions

The term “substituted” means that one or more hydrogen atoms on the designated atom or group are replaced with a selection from the indicated group, provided that the designated atom's normal valency under the existing circumstances is not exceeded. Combinations of substituents and/or variables are permissible.

The term “optionally substituted” means that the number of substituents can be equal to or different from zero. Unless otherwise indicated, it is possible that optionally substituted groups are substituted with as many optional substituents as can be accommodated by replacing a hydrogen atom with a non-hydrogen substituent on any available carbon or nitrogen atom. Commonly, it is possible for the number of optional substituents, when present, to be 1, 2, 3 or 4, in particular 1, 2 or 3.

When groups in the compounds according to the invention are substituted, it is possible for said groups to be mono-substituted or poly-substituted with substituent(s), unless otherwise specified. Within the scope of the present invention, the meanings of all groups which occur repeatedly are independent from one another. It is possible that groups in the compounds according to the invention are substituted with one, two or three identical or different substituents, particularly with one substituent.

As used herein, an oxo substituent represents an oxygen atom, which is bound to a carbon atom or to a sulfur atom via a double bond.

Should a composite substituent be composed of more than one part, e.g. (C₁-C₂-alkoxy)-(C₁-C₆-alkyl)-, it is possible for a given part to be attached at any suitable position of said composite substituent, e.g. it is possible for the C₁-C₂-alkoxy part to be attached to any suitable carbon atom of the C₁-C₆-alkyl part of said (C₁-C₂-alkoxy)-(C₁-C₆-alkyl)-group. A hyphen at the beginning or at the end of such a composite substituent indicates the point of attachment of said composite substituent to the rest of the molecule. Should a ring, comprising carbon atoms and optionally one or more heteroatoms, such as nitrogen, oxygen or sulfur atoms for example, be substituted with a substituent, it is possible for said substituent to be bound at any suitable position of said ring, be it bound to a suitable carbon atom and/or to a suitable heteroatom.

The term “comprising” when used in the specification includes “consisting of”.

If within the present text any item is referred to as “as mentioned herein”, it means that it may be mentioned anywhere in the present text.

The terms as mentioned in the present text have the following meanings:

The term “halogen atom” means a fluorine, chlorine, bromine or iodine atom, particularly a fluorine, chlorine or bromine atom.

The term “C₁-C₆-alkyl” means a linear or branched, saturated, monovalent hydrocarbon group having 1, 2, 3, 4, 5 or 6 carbon atoms, e.g. a methyl, ethyl, propyl, isopropyl, butyl, sec-butyl, isobutyl, tert-butyl, pentyl, isopentyl, 2-methylbutyl, 1-methylbutyl, 1-ethylpropyl, 1,2-dimethylpropyl, neo-pentyl, 1,1-dimethylpropyl, hexyl, 1-methylpentyl, 2-methylpentyl, 3-methylpentyl, 4-methylpentyl, 1-ethylbutyl, 2-ethylbutyl, 1,1-dimethylbutyl, 2,2-dimethylbutyl, 3,3-dimethylbutyl, 2,3-dimethylbutyl, 1,2-dimethylbutyl or 1,3-dimethylbutyl group, or an isomer thereof. Particularly, said group has 1, 2, 3 or 4 carbon atoms (“C₁-C₄-alkyl”), e.g. a methyl, ethyl, propyl, isopropyl, butyl, sec-butyl isobutyl, or tert-butyl group, more particularly 1, 2 or 3 carbon atoms (“C₁-C₃-alkyl”), e.g. a methyl, ethyl, n-propyl or isopropyl group, more particularly 1 or 2 carbon atoms (“C₁-C₂-alkyl”), e.g. a methyl or ethyl group.

1 or 2 carbon atoms (“C₁-C₂-alkyl”), e.g. a methyl or ethyl group.

The term “C₁-C₆-hydroxyalkyl” means a linear or branched, saturated, monovalent hydrocarbon group in which the term “C₁-C₆-alkyl” is defined supra, and in which 1 or 2 hydrogen atoms are replaced with a hydroxy group, e.g. a hydroxymethyl, 1-hydroxyethyl, 2-hydroxyethyl, 1,2-dihydroxyethyl, 3-hydroxypropyl, 2-hydroxypropyl, 1-hydroxypropyl, 1-hydroxypropan-2-yl, 2-hydroxypropan-2-yl, 2,3-dihydroxypropyl, 1,3-dihydroxypropan-2-yl, 3-hydroxy-2-methyl-propyl, 2-hydroxy-2-methyl-propyl, 1-hydroxy-2-methyl-propyl, 1-hydroxybutyl, 2-hydroxybutyl, 3-hydroxybutyl, 4-hydroxybutyl group, or an isomer thereof.

The term “C₁-C₆-haloalkyl” means a linear or branched, saturated, monovalent hydrocarbon group in which the term “C₁-C₆-alkyl” is as defined supra, and in which one or more of the hydrogen atoms are replaced, identically or differently, with a halogen atom. Particularly, said halogen atom is a fluorine atom. Said C₁-C₆-haloalkyl group is, for example, fluoromethyl, difluoromethyl, trifluoromethyl, 2-fluoroethyl, 2,2-difluoroethyl, 2,2,2-trifluoroethyl, pentafluoroethyl, 3,3,3-trifluoropropyl or 1,3-difluoropropan-2-yl.

The term “C₁-C₆-alkoxy” means a linear or branched, saturated, monovalent group of formula (C₁-C₆-alkyl)-O—, in which the term “C₁-C₆-alkyl” is as defined supra, e.g. a methoxy, ethoxy, n-propoxy, isopropoxy, n-butoxy, sec-butoxy, isobutoxy, tert-butoxy, pentyloxy, isopentyloxy or n-hexyloxy group, or an isomer thereof.

The term “C₁-C₆-haloalkoxy” means a linear or branched, saturated, monovalent C₁-C₆-alkoxy group, as defined supra, in which one or more of the hydrogen atoms is replaced, identically or differently, with a halogen atom. Particularly, said halogen atom is a fluorine atom. Said C₁-C₆-haloalkoxy group is, for example, fluoromethoxy, difluoromethoxy, trifluoromethoxy, 2,2,2-trifluoroethoxy or pentafluoroethoxy.

The term “C₂-C₆-alkenyl” means a linear or branched, monovalent hydrocarbon group, which contains one or two double bonds, and which has 2, 3, 4, 5 or 6 carbon atoms, it being understood that in the case in which said alkenyl group contains two double bonds, then it is possible for said double bonds to be conjugated with each other, or to form an allene. Said alkenyl group is, for example, an ethenyl (or “vinyl”), prop-2-en-1-yl (or “allyl”), prop-1-en-1-yl, but-3-enyl, but-2-enyl, but-1-enyl, pent-4-enyl, pent-3-enyl, pent-2-enyl, pent-1-enyl, hex-5-enyl, hex-4-enyl, hex-3-enyl, hex-2-enyl, hex-1-enyl, prop-1-en-2-yl (or “isopropenyl”), 2-methylprop-2-enyl, 1-methylprop-2-enyl, 2-methylprop-1-enyl, 1-methylprop-1-enyl, 3-methylbut-3-enyl, 2-methylbut-3-enyl, 1-methylbut-3-enyl, 3-methylbut-2-enyl, 2-methylbut-2-enyl, 1-methylbut-2-enyl, 3-methylbut-1-enyl, 2-methylbut-1-enyl, 1-methylbut-1-enyl, 1,1-dimethylprop-2-enyl, 1-ethylprop-1-enyl, 1-propylvinyl, 1-isopropylvinyl, 4-methylpent-4-enyl, 3-methylpent-4-enyl, 2-methylpent-4-enyl, 1-methylpent-4-enyl, 4-methylpent-3-enyl, 3-methylpent-3-enyl, 2-methylpent-3-enyl, 1-methylpent-3-enyl, 4-methylpent-2-enyl, 3-methylpent-2-enyl, 2-methylpent-2-enyl, 1-methylpent-2-enyl, 4-methylpent-1-enyl, 3-methylpent-1-enyl, 2-methylpent-1-enyl, 1-methylpent-1-enyl, 3-ethylbut-3-enyl, 2-ethylbut-3-enyl, 1-ethylbut-3-enyl, 3-ethylbut-2-enyl, 2-ethylbut-2-enyl, 1-ethylbut-2-enyl, 3-ethylbut-1-enyl, 2-ethylbut-1-enyl, 1-ethylbut-1-enyl, 2-propylprop-2-enyl, 1-propylprop-2-enyl, 2-isopropylprop-2-enyl, 1-isopropylprop-2-enyl, 2-propylprop-1-enyl, 1-propylprop-1-enyl, 2-isopropylprop-1-enyl, 1-isopropylprop-1-enyl, 3,3-dimethylprop-1-enyl, 1-(1,1-dimethylethyl)ethenyl, buta-1,3-dienyl, penta-1,4-dienyl or hexa-1,5-dienyl group.

The term “C₂-C₆-alkynyl” means a linear or branched, monovalent hydrocarbon group which contains one triple bond, and which contains 2, 3, 4, 5 or 6 carbon atoms, particularly 2,3 oder 4 carbon atoms (“C₂-C₄-alkynyl”). Said C₂-C₆-alkynyl group is, for example, ethynyl, prop-1-ynyl, prop-2-ynyl (or “propargyl”), but-1-ynyl, but-2-ynyl, but-3-ynyl, pent-1-ynyl, pent-2-ynyl, pent-3-ynyl, pent-4-ynyl, hex-1-ynyl, hex-2-ynyl, hex-3-ynyl, hex-4-ynyl, hex-5-ynyl, 1-methylprop-2-ynyl, 2-methylbut-3-ynyl, 1-methylbut-3-ynyl, 1-methylbut-2-ynyl, 3-methylbut-1-ynyl, 1-ethylprop-2-ynyl, 3-methylpent-4-ynyl, 2-methylpent-4-ynyl, 1-methyl-pent-4-ynyl, 2-methylpent-3-ynyl, 1-methylpent-3-ynyl, 4-methylpent-2-ynyl, 1-methyl-pent-2-ynyl, 4-methylpent-1-ynyl, 3-methylpent-1-ynyl, 2-ethylbut-3-ynyl, 1-ethylbut-3-ynyl, 1-ethylbut-2-ynyl, 1-propylprop-2-ynyl, 1-isopropylprop-2-ynyl, 2,2-dimethylbut-3-ynyl, 1,1-dimethylbut-3-ynyl, 1,1-dimethylbut-2-ynyl or 3,3-dimethylbut-1-ynyl group.

The term “C₃-C₆-cycloalkyl” means a saturated, monovalent, monocyclic hydrocarbon ring which contains 3, 4, 5 or 6 carbon atoms. Said C₃-C₆-cycloalkyl group is for example a cyclopropyl, cyclobutyl, cyclopentyl or cyclohexyl group. Particularly, said group has 3 or 4 carbon atoms (“C₃-C₄-cycloalkyl”), e.g. a cyclopropyl or cyclobutyl group.

The term “C₄-C₆-cycloalkenyl” means a monocyclic hydrocarbon ring which contains 4, 5 or 6 carbon atoms and one double bond. Particularly, said ring contains 5 or 6 carbon atoms (“C₅-C₆-cycloalkenyl”). Said C₄-C₆-cycloalkenyl group is for example, a monocyclic hydrocarbon ring, e.g. a cyclobutenyl, cyclopentenyl, cyclohexenyl or cycloheptenyll group. The term “C₃-C₆-cycloalkyloxy” means a saturated, monovalent group of formula (C₃-C₆-cycloalkyl)-O—, in which the term “C₃-C₆-cycloalkyl” is as defined supra, e.g. a cyclopropyloxy, cyclobutyloxy, cyclopentyloxy or cyclohexyloxy group.

The term “4- to 7-membered heterocycloalkyl” means a monocyclic, saturated heterocycle with 4, 5, 6 or 7 ring atoms in total, which contains one or two identical or different ring heteroatoms from the series N, O and S.

Said heterocycloalkyl group, without being limited thereto, can be a 4-membered ring, such as azetidinyl, oxetanyl or thietanyl, for example; or a 5-membered ring, such as tetrahydrofuranyl, 1,3-dioxolanyl, thiolanyl, pyrrolidinyl, imidazolidinyl, pyrazolidinyl, 1,1-dioxidothiolanyl, 1,2-oxazolidinyl, 1,3-oxazolidinyl or 1,3-thiazolidinyl, for example; or a 6-membered ring, such as tetrahydropyranyl, tetrahydrothiopyranyl, piperidinyl, morpholinyl, dithianyl, thiomorpholinyl, piperazinyl, 1,3-dioxanyl, 1,4-dioxanyl or 1,2-oxazinanyl, for example, or a 7-membered ring, such as azepanyl, 1,4-diazepanyl or 1,4-oxazepanyl, for example.

The term “5- to 7-membered heterocycloalkenyl” means a monocyclic, unsaturated, non-aromatic heterocycle with 5, 6 or 7 ring atoms in total, which contains one or two double bonds and one or two identical or different ring heteroatoms from the series N, O and S.

Said heterocycloalkenyl group is, for example, 4H-pyranyl, 2H-pyranyl, 2,5-dihydro-1H-pyrrolyl, [1,3]dioxolyl, 4H-[1,3,4]thiadiazinyl, 2,5-dihydrofuranyl, 2,3-dihydrofuranyl, 2,5-dihydrothio-phenyl, 2,3-dihydrothiophenyl, 4,5-dihydrooxazolyl or 4H-[1,4]thiazinyl.

The term “(4- to 7-membered heterocycloalkyl)oxy” means a monocyclic, saturated heterocycloalkyl of formula (4- to 7-membered heterocycloalkyl)-O— in which the term “4- to 7-membered heterocycloalkyl” is as defined supra.

The term “nitrogen containing 4- to 7-membered heterocycloalkyl group” means a monocyclic, saturated heterocycle with 4, 5, 6 or 7 ring atoms in total, which contains one ring nitrogen atom and optionally one further ring heteroatom from the series N, O and S.

Said nitrogen containing 4- to 7-membered heterocycloalkyl group, without being limited thereto, can be a 4-membered ring, such as azetidinyl, for example; or a 5-membered ring, such as pyrrolidinyl, imidazolidinyl, pyrazolidinyl, 1,2-oxazolidinyl, 1,3-oxazolidinyl or 1,3-thiazolidinyl, for example; or a 6-membered ring, such as piperidinyl, morpholinyl, thiomorpholinyl, piperazinyl, or 1,2-oxazinanyl, for example, or a 7-membered ring, such as azepanyl, 1,4-diazepanyl or 1,4-oxazepanyl, for example.

The term “heteroaryl” means a monovalent, monocyclic or bicyclic aromatic ring having 5, 6, 8, 9 or 10 ring atoms (a “5- to 10-membered heteroaryl” group), which contains at least one ring heteroatom and optionally one, two or three further ring heteroatoms from the series: N, O and/or S, and which is bound via a ring carbon atom.

Said heteroaryl group can be a 5-membered heteroaryl group, such as, for example, thienyl, furanyl, pyrrolyl, oxazolyl, thiazolyl, imidazolyl, pyrazolyl, isoxazolyl, isothiazolyl, oxadiazolyl, triazolyl, thiadiazolyl or tetrazolyl; or a 6-membered heteroaryl group, such as, for example, pyridinyl, pyridazinyl, pyrimidinyl, pyrazinyl or triazinyl; or a 9-membered heteroaryl group, such as, for example, benzofuranyl, benzothienyl, benzoxazolyl, benzisoxazolyl, benzimidazolyl, benzothiazolyl, benzotriazolyl, thiazolopyridinyl, indazolyl, indolyl, isoindolyl, indolizinyl or purinyl; or a 10-membered heteroaryl group, such as, for example, quinolinyl, quinazolinyl, isoquinolinyl, cinnolinyl, phthalazinyl, quinoxalinyl or pteridinyl.

In general, and unless otherwise mentioned, the heteroaryl or heteroarylene groups include all possible isomeric forms thereof, e.g.: tautomers and positional isomers with respect to the point of linkage to the rest of the molecule. Thus, for some illustrative non-restricting examples, the term pyridinyl includes pyridin-2-yl, pyridin-3-yl and pyridin-4-yl; or the term thienyl includes thien-2-yl and thien-3-yl.

The term “C₁-C₆”, as used in the present text, e.g. in the context of the definition of “C₁-C₆-alkyl”, “C₁-C₆-haloalkyl”, “C₁-C₆-hydroxyalkyl”, “C₁-C₆-alkoxy” or “C₁-C₆-haloalkoxy” means an alkyl group having a finite number of carbon atoms of 1 to 6, i.e. 1, 2, 3, 4, 5 or 6 carbon atoms.

Further, as used herein, the term “C₃-C₈”, as used in the present text, e.g. in the context of the definition of “C₃-C₆-cycloalkyl”, means a cycloalkyl group having a finite number of carbon atoms of 3 to 6, i.e. 3, 4, 5 or 6 carbon atoms.

When a range of values is given, said range encompasses each value and sub-range within said range.

For Example:

“C₁-C₆” encompasses C₁, C₂, C₃, C₄, C₅, C₆, C₁-C₆, C₁-C₅, C₁-C₄, C₁-C₃, C₁-C₂, C₂- C₆, C₂-C₅, C₂-C₄, C₂-C₃, C₃-C₆, C₃-C₅, C₃-C₄, C₄-C₆, C₄-C₅, and C₅-C₆;

“C₂-C₆” encompasses C₂, C₃, C₄, C₅, C₆, C₂-C₆, C₂-C₅, C₂-C₄, C₂-C₃, C₃-C₆, C₃-C₅, C₃-C₄, C₄-C₆, C₄-C₅, and C₅-C₆;

“C₃-C₆” encompasses C₃, C₄, C₅, C₆, C₃-C₆, C₃-C₅, C₃-C₄, C₄-C₆, C₄-C₅, and C₅-C₆.

As used herein, the term “leaving group” means an atom or a group of atoms that is displaced in a chemical reaction as stable species taking with it the bonding electrons. In particular, such a leaving group is selected from the group comprising: halide, in particular fluoride, chloride, bromide or iodide, (methylsulfonyl)oxy, [(trifluoromethyl)sulfonyl]oxy, [(nonafluorobutyl)-sulfonyl]oxy, (phenylsulfonyl)oxy, [(4-methylphenyl)sulfonyl]oxy, [(4-bromophenyl)sulfonyl]oxy, [(4-nitrophenyl)sulfonyl]oxy, [(2-nitrophenyl)sulfonyl]oxy, [(4-isopropylphenyl)sulfonyl]oxy, [(2,4,6-triisopropylphenyl)sulfonyl]oxy, [(2,4,6-trimethylphenyl)sulfonyl]oxy, [(4-tert-butyl-phenyl)sulfonyl]oxy and [(4-methoxyphenyl)sulfonyl]oxy.

It is possible for the compounds of general formula (I) to exist as isotopic variants. The invention therefore includes one or more isotopic variant(s) of the compounds of general formula (I), particularly deuterium-containing compounds of general formula (I).

The term “Isotopic variant” of a compound or a reagent is defined as a compound exhibiting an unnatural proportion of one or more of the isotopes that constitute such a compound.

The term “Isotopic variant of the compound of general formula (I)” is defined as a compound of general formula (I) exhibiting an unnatural proportion of one or more of the isotopes that constitute such a compound.

The expression “unnatural proportion” means a proportion of such isotope which is higher than its natural abundance. The natural abundances of isotopes to be applied in this context are described in “Isotopic Compositions of the Elements 1997”, Pure Appl. Chem., 70(1), 217-235, 1998.

Examples of such isotopes include stable and radioactive isotopes of hydrogen, carbon, nitrogen, oxygen, phosphorus, sulfur, fluorine, chlorine, bromine and iodine, such as ²H (deuterium), ³H (tritium), ¹¹C, ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁵I, ¹²⁹I and ¹³¹I, respectively.

With respect to the treatment and/or prophylaxis of the disorders specified herein the isotopic variant(s) of the compounds of general formula (I) preferably contain deuterium (“deuterium-containing compounds of general formula (I)”). Isotopic variants of the compounds of general formula (I) in which one or more radioactive isotopes, such as ³H or ¹⁴C, are incorporated are useful e.g. in drug and/or substrate tissue distribution studies. These isotopes are particularly preferred for the ease of their incorporation and detectability. Positron emitting isotopes such as ¹⁸F or ¹¹C may be incorporated into a compound of general formula (I). These isotopic variants of the compounds of general formula (I) are useful for in vivo imaging applications. Deuterium-containing and ¹³C-containing compounds of general formula (I) can be used in mass spectrometry analyses in the context of preclinical or clinical studies.

Isotopic variants of the compounds of general formula (I) can generally be prepared by methods known to a person skilled in the art, such as those described in the schemes and/or examples herein, by substituting a reagent for an isotopic variant of said reagent, preferably for a deuterium-containing reagent. Depending on the desired sites of deuteration, in some cases deuterium from D₂O can be incorporated either directly into the compounds or into reagents that are useful for synthesizing such compounds. Deuterium gas is also a useful reagent for incorporating deuterium into molecules. Catalytic deuteration of olefinic bonds and acetylenic bonds is a rapid route for incorporation of deuterium. Metal catalysts (i.e. Pd, Pt, and Rh) in the presence of deuterium gas can be used to directly exchange deuterium for hydrogen in functional groups containing hydrocarbons. A variety of deuterated reagents and synthetic building blocks are commercially available from companies such as for example C/D/N Isotopes, Quebec, Canada; Cambridge Isotope Laboratories Inc., Andover, Mass., USA; and CombiPhos Catalysts, Inc., Princeton, N.J., USA.

The term “deuterium-containing compound of general formula (I)” is defined as a compound of general formula (I), in which one or more hydrogen atom(s) is/are replaced by one or more deuterium atom(s) and in which the abundance of deuterium at each deuterated position of the compound of general formula (I) is higher than the natural abundance of deuterium, which is about 0.015%. Particularly, in a deuterium-containing compound of general formula (I) the abundance of deuterium at each deuterated position of the compound of general formula (I) is higher than 10%, 20%, 30%, 40%, 50%, 60%, 70% or 80%, preferably higher than 90%, 95%, 96% or 97%, even more preferably higher than 98% or 99% at said position(s). It is understood that the abundance of deuterium at each deuterated position is independent of the abundance of deuterium at other deuterated position(s).

The selective incorporation of one or more deuterium atom(s) into a compound of general formula (I) may alter the physicochemical properties (such as for example acidity [C. L. Perrin, et al., J. Am. Chem. Soc., 2007, 129, 4490], basicity [C. L. Perrin et al., J. Am. Chem. Soc., 2005, 127, 9641], lipophilicity [B. Testa et al., Int. J. Pharm., 1984, 19(3), 271]) and/or the metabolic profile of the molecule and may result in changes in the ratio of parent compound to metabolites or in the amounts of metabolites formed. Such changes may result in certain therapeutic advantages and hence may be preferred in some circumstances. Reduced rates of metabolism and metabolic switching, where the ratio of metabolites is changed, have been reported (A. E. Mutlib et al., Toxicol. Appl. Pharmacol., 2000, 169, 102). These changes in the exposure to parent drug and metabolites can have important consequences with respect to the pharmacodynamics, tolerability and efficacy of a deuterium-containing compound of general formula (I). In some cases deuterium substitution reduces or eliminates the formation of an undesired or toxic metabolite and enhances the formation of a desired metabolite (e.g. Nevirapine: A. M. Sharma et al., Chem. Res. Toxicol., 2013, 26, 410; Efavirenz: A. E. Mutlib et al., Toxicol. Appl. Pharmacol., 2000, 169, 102). In other cases the major effect of deuteration is to reduce the rate of systemic clearance. As a result, the biological half-life of the compound is increased. The potential clinical benefits would include the ability to maintain similar systemic exposure with decreased peak levels and increased trough levels. This could result in lower side effects and enhanced efficacy, depending on the particular compound's pharmacokinetic/pharmacodynamic relationship. ML-337 (C. J. Wenthur et al., J. Med. Chem., 2013, 56, 5208) and Odanacatib (K. Kassahun et al., WO2012/112363) are examples for this deuterium effect. Still other cases have been reported in which reduced rates of metabolism result in an increase in exposure of the drug without changing the rate of systemic clearance (e.g. Rofecoxib: F. Schneider et al., Arzneim. Forsch./Drug. Res., 2006, 56, 295; Telaprevir: F. Maltais et al., J. Med. Chem., 2009, 52, 7993). Deuterated drugs showing this effect may have reduced dosing requirements (e.g. lower number of doses or lower dosage to achieve the desired effect) and/or may produce lower metabolite loads.

A compound of general formula (I) may have multiple potential sites of attack for metabolism. To optimize the above-described effects on physicochemical properties and metabolic profile, deuterium-containing compounds of general formula (I) having a certain pattern of one or more deuterium-hydrogen exchange(s) can be selected. Particularly, the deuterium atom(s) of deuterium-containing compound(s) of general formula (I) is/are attached to a carbon atom and/or is/are located at those positions of the compound of general formula (I), which are sites of attack for metabolizing enzymes such as e.g. cytochrome P₄₅₀.

In another embodiment the present invention concerns a deuterium-containing compound of general formula (I) having 1, 2, 3 or 4 deuterium atoms, particularly with 1, 2 or 3 deuterium atoms.

Where the plural form of the word compounds, salts, polymorphs, hydrates, solvates and the like, is used herein, this is taken to mean also a single compound, salt, polymorph, isomer, hydrate, solvate or the like.

By “stable compound’ or “stable structure” is meant a compound that is sufficiently robust to survive isolation to a useful degree of purity from a reaction mixture, and formulation into an efficacious therapeutic agent.

The compounds of the present invention optionally contain one or more asymmetric centres, depending upon the location and nature of the various substituents desired. It is possible that one or more asymmetric carbon atoms are present in the (R) or (S) configuration, which can result in racemic mixtures in the case of a single asymmetric centre, and in diastereomeric mixtures in the case of multiple asymmetric centres. In certain instances, it is possible that asymmetry also be present due to restricted rotation about a given bond, for example, the central bond adjoining two substituted aromatic rings of the specified compounds.

Preferred compounds are those which produce the more desirable biological activity. Separated, pure or partially purified isomers and stereoisomers or racemic or diastereomeric mixtures of the compounds of the present invention are also included within the scope of the present invention. The purification and the separation of such materials can be accomplished by standard techniques known in the art.

Preferred compounds are those which produce the more desirable biological activity. Separated, pure or partially purified isomers and stereoisomers or racemic or diastereomeric mixtures of the compounds of the present invention are also included within the scope of the present invention. The purification and the separation of such materials can be accomplished by standard techniques known in the art.

The optical isomers can be obtained by resolution of the racemic mixtures according to conventional processes, for example, by the formation of diastereoisomeric salts using an optically active acid or base or formation of covalent diastereomers. Examples of appropriate acids are tartaric, diacetyltartaric, ditoluoyltartaric and camphorsulfonic acid. Mixtures of diastereoisomers can be separated into their individual diastereomers on the basis of their physical and/or chemical differences by methods known in the art, for example, by chromatography or fractional crystallisation. The optically active bases or acids are then liberated from the separated diastereomeric salts. A different process for separation of optical isomers involves the use of chiral chromatography (e.g., HPLC columns using a chiral phase), with or without conventional derivatisation, optimally chosen to maximise the separation of the enantiomers. Suitable HPLC columns using a chiral phase are commercially available, such as those manufactured by Daicel, e.g., Chiracel OD and Chiracel OJ, for example, among many others, which are all routinely selectable. Enzymatic separations, with or without derivatisation, are also useful. The optically active compounds of the present invention can likewise be obtained by chiral syntheses utilizing optically active starting materials.

In order to distinguish different types of isomers from each other reference is made to IUPAC Rules Section E (Pure Appl Chem 45, 11-30, 1976).

The present invention includes all possible stereoisomers of the compounds of the present invention as single stereoisomers, or as any mixture of said stereoisomers, e.g. (R)- or (S)-isomers, in any ratio. Isolation of a single stereoisomer, e.g. a single enantiomer or a single diastereomer, of a compound of the present invention is achieved by any suitable state of the art method, such as chromatography, especially chiral chromatography, for example.

Further, it is possible for the compounds of the present invention to exist as tautomers. For example, the compounds of the present invention may contain an amide moiety and can exist as an amide, or an imidic acid, or even a mixture in any amount of the two tautomers, namely

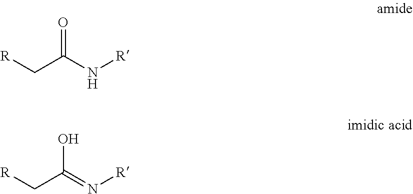

The present invention includes all possible tautomers of the compounds of the present invention as single tautomers, or as any mixture of said tautomers, in any ratio. Further, the compounds of the present invention can exist as N-oxides, which are defined in that at least one nitrogen of the compounds of the present invention is oxidised. The present invention includes all such possible N-oxides.

The present invention also covers useful forms of the compounds of the present invention, such as metabolites, hydrates, solvates, prodrugs, salts, in particular pharmaceutically acceptable salts, and/or co-precipitates.

The compounds of the present invention can exist as a hydrate, or as a solvate, wherein the compounds of the present invention contain polar solvents, in particular water, methanol or ethanol for example, as structural element of the crystal lattice of the compounds. It is possible for the amount of polar solvents, in particular water, to exist in a stoichiometric or non-stoichiometric ratio. In the case of stoichiometric solvates, e.g. a hydrate, hemi-, (semi-), mono-, sesqui-, di-, tri-, tetra-, penta- etc. solvates or hydrates, respectively, are possible. The present invention includes all such hydrates or solvates.

Further, it is possible for the compounds of the present invention to exist in free form, e.g. as a free base, or as a free acid, or as a zwitterion, or to exist in the form of a salt. Said salt may be any salt, either an organic or inorganic addition salt, particularly any pharmaceutically acceptable organic or inorganic addition salt, which is customarily used in pharmacy, or which is used, for example, for isolating or purifying the compounds of the present invention. The term “pharmaceutically acceptable salt” refers to an inorganic or organic acid addition salt of a compound of the present invention. For example, see S. M. Berge, et al. “Pharmaceutical Salts,” J. Pharm. Sci. 1977, 66, 1-19.

A suitable pharmaceutically acceptable salt of the compounds of the present invention may be, for example, an acid-addition salt of a compound of the present invention bearing a nitrogen atom, in a chain or in a ring, for example, which is sufficiently basic, such as an acid-addition salt with an inorganic acid, or “mineral acid”, such as hydrochloric, hydrobromic, hydroiodic, sulfuric, sulfamic, bisulfuric, phosphoric, or nitric acid, for example, or with an organic acid, such as formic, acetic, acetoacetic, pyruvic, trifluoroacetic, propionic, butyric, hexanoic, heptanoic, undecanoic, lauric, benzoic, salicylic, 2-(4-hydroxybenzoyl)-benzoic, camphoric, cinnamic, cyclopentanepropionic, digluconic, 3-hydroxy-2-naphthoic, nicotinic, pamoic, pectinic, 3-phenylpropionic, pivalic, 2-hydroxyethanesulfonic, itaconic, trifluoromethanesulfonic, dodecylsulfuric, ethanesulfonic, benzenesulfonic, para-toluenesulfonic, methanesulfonic, 2-naphthalenesulfonic, naphthalinedisulfonic, camphorsulfonic acid, citric, tartaric, stearic, lactic, oxalic, malonic, succinic, malic, adipic, alginic, maleic, fumaric, D-gluconic, mandelic, ascorbic, glucoheptanoic, glycerophosphoric, aspartic, sulfosalicylic, or thiocyanic acid, for example.

Further, another suitably pharmaceutically acceptable salt of a compound of the present invention which is sufficiently acidic, is an alkali metal salt, for example a sodium or potassium salt, an alkaline earth metal salt, for example a calcium, magnesium or strontium salt, or an aluminium or a zinc salt, or an ammonium salt derived from ammonia or from an organic primary, secondary or tertiary amine having 1 to 20 carbon atoms, such as ethylamine, diethylamine, triethylamine, ethyldiisopropylamine, monoethanolamine, diethanolamine, triethanolamine, dicyclohexylamine, dimethylaminoethanol, diethylaminoethanol, tris(hydroxymethyl)aminomethane, procaine, dibenzylamine, N-methylmorpholine, arginine, lysine, 1,2-ethylenediamine, N-methylpiperidine, N-methyl-glucamine, N,N-dimethyl-glucamine, N-ethyl-glucamine, 1,6-hexanediamine, glucosamine, sarcosine, serinol, 2-amino-1,3-propanediol, 3-amino-1,2-propanediol, 4-amino-1,2,3-butanetriol, or a salt with a quarternary ammonium ion having 1 to 20 carbon atoms, such as tetramethylammonium, tetraethylammonium, tetra(n-propyl)ammonium, tetra(n-butyl)ammonium, N-benzyl-N,N,N-trimethylammonium, choline or benzalkonium.

Those skilled in the art will further recognise that it is possible for acid addition salts of the claimed compounds to be prepared by reaction of the compounds with the appropriate inorganic or organic acid via any of a number of known methods. Alternatively, alkali and alkaline earth metal salts of acidic compounds of the present invention are prepared by reacting the compounds of the present invention with the appropriate base via a variety of known methods.

The present invention includes all possible salts of the compounds of the present invention as single salts, or as any mixture of said salts, in any ratio.

In the present text, in particular in the “Experimental Section”, for the synthesis of intermediates and of examples of the present invention, when a compound is mentioned as a salt form with the corresponding base or acid, the exact stoichiometric composition of said salt form, as obtained by the respective preparation and/or purification process, is, in most cases, unknown.

Unless specified otherwise, suffixes to chemical names or structural formulae relating to salts, such as “hydrochloride”, “trifluoroacetate”, “sodium salt”, or “x HCl”, “x CF₃COOH”, “x Na⁺”, for example, mean a salt form, the stoichiometry of which salt form not being specified.

This applies analogously to cases in which synthesis intermediates or example compounds or salts thereof have been obtained, by the preparation and/or purification processes described, as solvates, such as hydrates, with (if defined) unknown stoichiometric composition.

Furthermore, the present invention includes all possible crystalline forms, or polymorphs, of the compounds of the present invention, either as single polymorph, or as a mixture of more than one polymorph, in any ratio.

Moreover, the present invention also includes prodrugs of the compounds according to the invention. The term “prodrugs” here designates compounds which themselves can be biologically active or inactive, but are converted (for example metabolically or hydrolytically) into compounds according to the invention during their residence time in the body.

The invention further includes all possible cyclodextrin clathrates, i.e alpha-, beta-, or gamma-cyclodextrins, hydroxypropyl-beta-cyclodextrins, methylbetacyclodextrins.

In accordance with a second embodiment of the first aspect, the present invention covers compounds of general formula (I), supra, in which:
n represents an integer selected from 1, 2 and 3,
X represents a group selected from
   —O—, —S—, —S(═O)—, —S(═O)₂—, *-(C(R¹⁶)(R¹⁷))ₒ-#, *-O—C(R¹⁶)₂—(C(R¹⁶)(R¹⁷))ₚ-#, *-(C(R¹⁶)(R¹⁷))ₚ—C(R¹⁶)₂—O—#,

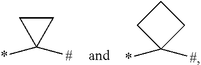

wherein * indicates the point of attachment to R² and #indicates the point of attachment to the pyrrolidine-, piperidine- and azepane moiety,
R¹ represents a group selected from cyano, —C(═O)NH₂, —C(═O)N(H)CH₃, —C(═O)N(H)C₂H₅, —C(═O)N(CH₃)₂ and —C(═O)OR¹⁵,
R² represents a group selected from phenyl, naphthyl and 5- to 10-membered heteroaryl,
   which 5- to 10-membered heteroaryl group is connected to the rest of the molecule via a carbon atom of said 5- to 10-membered heteroaryl group,
   and
   which phenyl, naphthyl and 5- to 10-membered heteroaryl group is optionally substituted, one, two, three or four times, each substituent independently selected from a halogen atom or a group selected from
   C₁-C₄-alkyl, C₃-C₅-cycloalkyl, C₁-C₄-hydroxyalkyl, C₁-C₄-haloalkyl, (C₁-C₂-alkoxy)-(C₁-C₄-alkyl)-, C₁-C₄-alkoxy, (C₁-C₂-alkoxy)-(C₁-C₄-alkoxy)-, C₁-C₄-haloalkoxy, C₃-C₅-cycloalkyloxy, phenoxy, —SR¹⁴, —S(═O)R¹⁴, —S(═O)₂R¹⁴, —P(═O)(R¹⁴)₂, nitro, cyano, hydroxy, —N(R⁹)(R¹⁰), —C(═O)N(R⁹)(R¹⁰), —C(═O)R¹¹, —C(═O)₂R¹⁵, —N(R¹²)C(═O)R¹³, —N(R¹²)S(═O)₂R¹⁴, —N═S(═NH)(R¹⁴)₂, —N═S(═O)(R¹⁴)₂, —S(═O)(═NR¹⁴)R¹³, 4- to 7-membered heterocycloalkyl, 5- to 7-membered heterocycloalkenyl, (4- to 7-membered heterocycloalkyl)oxy, phenyl and 5- or 6-membered heteroaryl,
   or two substituents of said phenyl group, when they are attached to adjacent ring atoms, are optionally linked to one another in such a way that they jointly form a group selected from
   —(CH₂)₃—, —(CH₂)₄—, —O—(CH₂)₂—, —(CH₂)₂—O—, —CH₂—O—CH₂—, —O—(CH₂)₃—, —(CH₂)₃—O—, —CH₂—O—(CH₂)₂—, —(CH₂)₂O—CH₂—, —O—CH₂—O— and —O—(CH₂)₂—O—,
      wherein said 4- to 7-membered heterocycloalkyl group and
      5- to 7-membered heterocycloalkenyl group is connected to the rest of the molecule via a carbon atom of said
      4- to 7-membered heterocycloalkyl group and
      5- to 7-membered heterocycloalkenyl group,
      and
      wherein said 4- to 7-membered heterocycloalkyl group,
      5- to 7-membered heterocycloalkenyl group and
      (4- to 7-membered heterocycloalkyl)oxy group is optionally substituted, one, two or three times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl, —N(R⁹)(R¹⁰) and oxo,
      and
      wherein said C₁-C₄-alkyl and C₁-C₄-alkoxy group is optionally substituted with a group selected from
      C₃-C₄-cycloalkyl, phenyl and 4- to 7-membered heterocycloalkyl,
         wherein said 4- to 7-membered heterocycloalkyl group is connected to the rest of the molecule via a carbon atom of said 4- to 7-membered heterocycloalkyl group,
         and
         wherein said 4- to 7-membered heterocycloalkyl group is optionally substituted, one, two or three times, each substituent independently selected from a halogen atom or a group selected from
         C₁-C₂-alkyl, C₁-C₂-haloalkyl, cyano, hydroxy, C₁-C₂-alkoxy, C₃-C₄-cycloalkyl, —N(R⁹)(R¹⁰) and oxo,
         and
         which phenyl group is optionally substituted, one or two times, each substituent independently selected from a halogen atom or a group selected from
         C₁-C₂-alkyl, C₁-C₂-haloalkyl, cyano, hydroxy, C₁-C₂-alkoxy, C₃-C₄-cycloalkyl and —N(R⁹)(R¹⁰),
         and
         which C₃-C₄-cycloalkyl group is optionally substituted, one or two times, each substituent independently selected from a halogen atom or a group selected from cyano and hydroxy,
      and
      wherein said C₃-C₅-cycloalkyl group is optionally substituted, one or two times, each substituent independently selected from a halogen atom or a C₁-C₄-alkyl group,
      and
      wherein said phenyl, phenoxy and 5- or 6-membered heteroaryl group is optionally substituted, one or two times, each substituent independently selected from a halogen atom or a group selected from
      C₁-C₂-alkyl, C₁-C₂-haloalkyl, cyano, hydroxy, C₁-C₂-alkoxy,
      C₃-C₄-cycloalkyl and —N(R⁹)(R¹⁰),
R³ represents a hydrogen atom or a halogen atom or a group selected from
   C₁-C₄-alkyl, C₂-C₄-alkenyl, C₂-C₄-alkynyl, C₃-C₅-cycloalkyl, C₄-C₅-cycloalkenyl, C₁-C₄-hydroxyalkyl, C₁-C₄-haloalkyl, (C₁-C₂-alkoxy)-(C₁-C₄-alkyl)-, C₁-C₄-alkoxy, (C₁-C₂-alkoxy)-(C₁-C₄-alkoxy)-, C₁-C₄-haloalkoxy, C₃-C₅-cycloalkyloxy, phenoxy, —SR¹⁴, —S(═O)R¹⁴, —S(═O)₂R¹⁴, cyano, hydroxy, —N(R⁹)(R¹⁰), —C(═O)N(R⁹)(R¹⁰), —C(═O)R¹¹, —N(R¹²)C(═O)R¹³, —N(R¹²)S(═O)₂R¹⁴, —N═S(═NH)(R¹⁴)₂, —N═S(═O)(R¹⁴)₂, —P(═O)(R¹⁴)₂, 4- to 7-membered heterocycloalkyl, 5- to 7-membered heterocycloalkenyl, (4- to 7-membered heterocycloalkyl)oxy, phenyl and 5- or 6-membered heteroaryl,
      wherein said 4- to 7-membered heterocycloalkyl group and
      5- to 7-membered heterocycloalkenyl group is connected to the rest of the molecule via a carbon atom of said
      4- to 7-membered heterocycloalkyl group and
      5- to 7-membered heterocycloalkenyl group,
      and
      wherein said 4- to 7-membered heterocycloalkyl group,
      5- to 7-membered heterocycloalkenyl group and
      (4- to 7-membered heterocycloalkyl)oxy group is optionally substituted, one, two or three times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl, —N(R⁹)(R¹⁰) and oxo,
      and
      wherein said C₁-C₄-alkyl, C₂-C₄-alkenyl, C₂-C₄-alkynyl and C₁-C₄-alkoxy group is optionally substituted with a group selected from
      C₃-C₄-cycloalkyl, phenyl and 4- to 7-membered heterocycloalkyl,
         wherein said 4- to 7-membered heterocycloalkyl group, is connected to the rest of the molecule via a carbon atom of said 4- to 7-membered heterocycloalkyl group,
         and
         wherein said 4- to 7-membered heterocycloalkyl group is optionally substituted, one, two or three times, each substituent independently selected from a halogen atom or a group selected from
         C₁-C₂-alkyl, C₁-C₂-haloalkyl, cyano, hydroxy, C₁-C₂-alkoxy,
         C₃-C₄-cycloalkyl, —N(R⁹)(R¹⁰) and oxo,
         and
         which phenyl group is optionally substituted, one or two times, each substituent independently selected from a halogen atom or a group selected from
         C₁-C₂-alkyl, C₁-C₂-haloalkyl, cyano, hydroxy, C₁-C₂-alkoxy,
         C₃-C₄-cycloalkyl and —N(R⁹)(R¹⁰),
         and
         which C₃-C₄-cycloalkyl group is optionally substituted, one or two times, each substituent independently selected from a halogen atom or a group selected from
         cyano and hydroxy,
      and
      wherein said C₃-C₅-cycloalkyl and C₄-C₅-cycloalkenyl group is optionally substituted, one or two times, each substituent independently selected from a halogen atom or a C₁-C₄-alkyl group,
      and
      wherein said phenyl, phenoxy and 5- or 6-membered heteroaryl group is optionally substituted, one or two times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl and —N(R⁹)(R¹⁰),
R⁴ represents a hydrogen atom or a halogen atom or a group selected from
   C₁-C₄-alkyl, C₂-C₄-alkenyl, C₂-C₄-alkynyl, C₃-C₅-cycloalkyl, C₄-C₅-cycloalkenyl, C₁-C₄-hydroxyalkyl, C₁-C₄-haloalkyl, (C₁-C₂-alkoxy)-(C₁-C₄-alkyl)-, C₁-C₄-alkoxy, (C₁-C₂-alkoxy)-(C₁-C₄-alkoxy)-, C₁-C₄-haloalkoxy, C₃-C₅-cycloalkyloxy, —S(═O)R¹⁴, —S(═O)₂R¹⁴, cyano, hydroxy, —N(R⁹)(R¹⁰), —N(R¹⁸)(R¹⁹), —C(═O)N(R⁹)(R¹⁰), —C(═O)R¹¹, —N(R¹²)C(═O)R¹³, —N(R¹²)S(═O)₂R¹⁴, —N═S(═NH)(R¹⁴)₂, —N═S(═O)(R¹⁴)₂, —P(═O)(R¹⁴)₂, 4- to 7-membered heterocycloalkyl, 5- to 7-membered heterocycloalkenyl, (4- to 7-membered heterocycloalkyl)oxy, phenyl and 5- or 6-membered heteroaryl,
      wherein said 4- to 7-membered heterocycloalkyl group and
      5- to 7-membered heterocycloalkenyl group is connected to the rest of the molecule via a carbon atom of said 4- to 7-membered heterocycloalkyl group and 5- to 7-membered heterocycloalkenyl group,
      and
      wherein said 4- to 7-membered heterocycloalkyl group,
      5- to 7-membered heterocycloalkenyl group and (4- to 7-membered heterocycloalkyl)oxy group is optionally substituted, one, two or three times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl, —N(R⁹)(R¹⁰) and oxo,
      and
      wherein said C₁-C₄-alkyl, C₂-C₄-alkenyl, C₂-C₄-alkynyl and C₁-C₄-alkoxy group is optionally substituted with a group selected from
      C₃-C₄-cycloalkyl, phenyl and 4- to 7-membered heterocycloalkyl,
         wherein said 4- to 7-membered heterocycloalkyl group is connected to the rest of the molecule via a carbon atom of said 4- to 7-membered heterocycloalkyl group,
         and
         wherein said 4- to 7-membered heterocycloalkyl group is optionally substituted, one, two or three times, each substituent independently selected from a halogen atom or a group selected from
         C₁-C₂-alkyl, C₁-C₂-haloalkyl, cyano, hydroxy, C₁-C₂-alkoxy, C₃-C₄-cycloalkyl, —N(R⁹)(R¹⁰) and oxo,
         and
         which phenyl group is optionally substituted, one or two times, each substituent independently selected from a halogen atom or a group selected from
         C₁-C₂-alkyl, C₁-C₂-haloalkyl, cyano, hydroxy, C₁-C₂-alkoxy, C₃-C₄-cycloalkyl and —N(R⁹)(R¹⁰),
         and
         which C₃-C₄-cycloalkyl group is optionally substituted, one or two times, each substituent independently selected from a halogen atom or a group selected from
         cyano and hydroxy,
      and
      wherein said C₃-C₅-cycloalkyl and C₄-C₅-cycloalkenyl group is optionally substituted, one or two times, each substituent independently selected from a halogen atom or a C₁-C₄-alkyl group,
      and
      wherein said phenyl and 5- or 6-membered heteroaryl group is optionally substituted, one or two times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl and —N(R⁹)(R¹⁰),
R⁵ represents a hydrogen atom or a halogen atom or a group selected from
   C₁-C₄-alkyl, C₂-C₄-alkenyl, C₂-C₄-alkynyl, C₃-C₅-cycloalkyl, C₄-C₅-cycloalkenyl, C₁-C₄-hydroxyalkyl, C₁-C₄-haloalkyl, (C₁-C₂-alkoxy)-(C₁-C₄-alkyl)-, C₁-C₄-alkoxy, (C₁-C₂-alkoxy)-(C₁-C₄-alkoxy), —C₁-C₄-haloalkoxy, C₃-C₅-cycloalkyloxy, phenoxy, —SR¹⁴, —S(═O)R¹⁴, S(═O)₂R¹⁴, cyano, hydroxy, —N(R⁹)(R¹⁰), —C(═O)N(R⁹)(R¹⁰), —C(═O)R¹¹, —N(R¹²)C(═O)R¹³, —N(R¹²)S(═O)₂R¹⁴, —N═S(═NH)(R¹⁴)₂, —N═S(═O)(R¹⁴)₂, —P(═O)(R¹⁴)₂, 4- to 7-membered heterocycloalkyl, 5- to 7-membered heterocycloalkenyl,
   (4- to 7-membered heterocycloalkyl)oxy, phenyl and 5- or 6-membered heteroaryl,
      wherein said 4- to 7-membered heterocycloalkyl group and
      5- to 7-membered heterocycloalkenyl group is connected to the rest of the molecule via a carbon atom of said
      4- to 7-membered heterocycloalkyl group and
      5- to 7-membered heterocycloalkenyl group,
      and
      wherein said 4- to 7-membered heterocycloalkyl group,
      5- to 7-membered heterocycloalkenyl group and
      (4- to 7-membered heterocycloalkyl)oxy group is optionally substituted, one, two or three times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl, —N(R⁹)(R¹⁰) and oxo,
      and
      wherein said C₁-C₄-alkyl, C₂-C₄-alkenyl, C₂-C₄-alkynyl and C₁-C₄-alkoxy group is optionally substituted with a group selected from
      C₃-C₄-cycloalkyl, phenyl and 4- to 7-membered heterocycloalkyl,
         wherein said 4- to 7-membered heterocycloalkyl group is connected to the rest of the molecule via a carbon atom of said
         4- to 7-membered heterocycloalkyl group,
         and
         wherein said 4- to 7-membered heterocycloalkyl group is optionally substituted, one, two or three times, each substituent independently selected from a halogen atom or a group selected from
         C₁-C₂-alkyl, C₁-C₂-haloalkyl, cyano, hydroxy, C₁-C₂-alkoxy, C₃-C₄-cycloalkyl, —N(R⁹)(R¹⁰) and oxo,
         and
         which phenyl group is optionally substituted, one or two times, each substituent independently selected from a halogen atom or a group selected from
         C₁-C₂-alkyl, C₁-C₂-haloalkyl, cyano, hydroxy, C₁-C₂-alkoxy, C₃-C₄-cycloalkyl and —N(R⁹)(R¹⁰),
         and
         which C₃-C₄-cycloalkyl group is optionally substituted, one or two times, each substituent independently selected from a halogen atom or a group selected from
         cyano and hydroxy,
      and
      wherein said C₃-C₅-cycloalkyl and C₄-C₅-cycloalkenyl group is optionally substituted, one or two times, each substituent independently selected from a halogen atom or a C₁-C₄-alkyl group,
      and
      wherein said phenyl, phenoxy and 5- or 6-membered heteroaryl group is optionally substituted, one or two times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl and —N(R⁹)(R¹⁰),
R⁶ represents a hydrogen atom, or a fluorine atom or a group selected from
   C₁-C₄-alkyl, C₁-C₄-hydroxyalkyl, C₁-C₄-alkoxy, hydroxy and oxo,
R⁷ represents a hydrogen atom or a halogen atom or a group selected from
   C₁-C₄-alkyl, C₁-C₄-alkoxy, hydroxy and cyano,
R³ represents a group selected from methyl and ethyl,
R⁹ and R¹⁰ represent, independently from each occurrence, a hydrogen atom or a group selected from
   C₁-C₄-alkyl, (C₁-C₄-alkoxy)-(C₂-C₄-alkyl)-, C₃-C₄-cycloalkyl and C₂-C₄-haloalkyl,
   or
R⁹ and R¹⁰ together with the nitrogen to which they are attached represent a nitrogen containing 4- to 7-membered heterocycloalkyl group or 5- to 7-membered heterocycloalkenyl group,
   wherein said nitrogen containing 4- to 7-membered heterocycloalkyl group and 5- to 7-membered heterocycloalkenyl group are optionally substituted, one, two or three times, each substituent independently selected from a halogen atom or a group selected from
   C₁-C₄-alkyl, C₃-C₄-cycloalkyl, hydroxy and oxo,
   or
   two substituents, which are attached to the same carbon atom of said nitrogen containing 4- to 7-membered heterocycloalkyl group, together with the carbon atom to which they are attached, represent a 4- to 7-membered heterocycloalkyl group,
      wherein said 4- to 7-membered heterocycloalkyl group is optionally substituted, one or two times, each substituent independently selected from a halogen atom or a group selected from
      C₁-C₄-alkyl, C₃-C₄-cycloalkyl, C₁-C₄-haloalkyl, hydroxy and oxo,
R¹¹ represents a hydrogen atom or group selected from C₁-C₄-alkyl, C₁-C₄-hydroxyalkyl, C₁-C₄-haloalkyl, phenyl and 5- or 6-membered heteroaryl,
   wherein said phenyl group and 5- or 6-membered heteroaryl group is optionally substituted, one or two times, each substituent independently selected from a halogen atom or a group selected from
   C₁-C₂-alkyl, C₁-C₂-haloalkyl, cyano, hydroxy, C₁-C₂-alkoxy, C₃-C₄-cycloalkyl and —N(R⁹)(R¹⁰),
R¹² represents a hydrogen atom or a C₁-C₄-alkyl group,
R¹³ represents a hydrogen atom or a group selected from
   C₁-C₄-alkyl, phenyl and 5- or 6-membered heteroaryl,
      wherein said phenyl group and 5- or 6-membered heteroaryl group is optionally substituted, one or two times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl and —N(R⁹)(R¹⁰),
R¹⁴ represents a group selected from C₁-C₄-alkyl, C₁-C₄-haloalkyl, C₃-C₅-cycloalkyl, phenyl and 5- or 6-membered heteroaryl,
   wherein said phenyl group and 5- or 6-membered heteroaryl group is optionally substituted, one or two times, each substituent independently selected from a halogen atom or a group selected from
   C₁-C₂-alkyl, C₁-C₂-haloalkyl, cyano, hydroxy, C₁-C₂-alkoxy, C₃-C₄-cycloalkyl and —N(R⁹)(R¹⁰)
R¹⁵ represents a hydrogen atom or a C₁-C₄-alkyl group,
R¹⁶ represent, indepently from each other, a hydrogen atom or a halogen atom or a group selected from
   C₁-C₂-alkyl and C₁-C₂-haloalkyl,
R¹⁷ represent, indepently from each other, a hydrogen atom or a halogen atom or a group selected from
   C₁-C₂-alkyl, C₁-C₂-haloalkyl, C₁-C₂-alkoxy, C₁-C₂-haloalkoxy and hydroxy,
R¹⁸ represents a hydrogen atom or a group selected from
   C₁-C₄-alkyl, C₃-C₄-cycloalkyl and C₂-C₄-haloalkyl,
R¹⁹ represents a 4- to 7-membered heterocycloalkyl group,
   wherein said 4- to 7-membered heterocycloalkyl group is optionally substituted, one, two or three times, each substituent independently selected from a halogen atom or a group selected from
   C₁-C₄-alkyl, C₃-C₄-cycloalkyl, C₁-C₄-alkoxy, hydroxy and oxo,
   and
   wherein said 4- to 7-membered heterocycloalkyl group is connected to the rest of the molecule via a carbon atom of the 4- to 7-membered heterocycloalkyl group,
o represents an integer selected from 1 or 2,
and
p represents an integer selected from 0 and 1,
and stereoisomers, tautomers, N-oxides, hydrates, solvates, and salts thereof, and mixtures of same.
In accordance with a third embodiment of the first aspect, the present invention covers compounds of general formula (I), supra, in which:
n represents an integer selected from 1, 2 and 3,
X represents a group selected from
   —O—, —S—, —S(═O)—, —S(═O)₂—, *-(C(R¹⁶)(R⁷))ₒ-#, *-O—C(R¹⁶)₂—(C(R¹⁶)(R⁷))ₚ-#, *-(C(R¹⁶)(R¹⁷))ₚ—C(R¹⁶)₂—O—#,

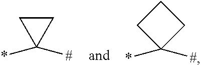

wherein * indicates the point of attachment to R² and #indicates the point of attachment to the pyrrolidine-, piperidine- and azepane moiety,
R¹ represents a group selected from cyano, —C(═O)NH₂, —C(═O)N(H)CH₃, —C(═O)N(H)C₂H₅, —C(═O)N(CH₃)₂ and —C(═O)OR¹⁵,
R² represents a group selected from phenyl, naphthyl and 5- to 10-membered heteroaryl,
   which 5- to 10-membered heteroaryl group is connected to the rest of the molecule via a carbon atom of said 5- to 10-membered heteroaryl group,
   and
   which phenyl, naphthyl and 5- to 10-membered heteroaryl group is optionally substituted, one, two, three or four times, each substituent independently selected from a halogen atom or a group selected from
   C₁-C₄-alkyl, C₃-C₅-cycloalkyl, C₁-C₄-haloalkyl, C₁-C₄-alkoxy, (C₁-C₂-alkoxy)-(C₁-C₄-alkoxy)-, C₁-C₄-haloalkoxy, —SR¹⁴, —S(═O)R¹⁴, —S(═O)₂R¹⁴, —P(═O)(R¹⁴)₂, nitro, cyano, hydroxy, —N(R⁹)(R¹⁰), —C(═O)N(R⁹)(R¹⁰), —C(═O)₂R¹⁵, —N(R¹²)S(═O)₂R¹⁴, —N═S(═O)(R¹⁴)₂, —S(═O)(═NR¹⁴)R¹³,
   4- to 7-membered heterocycloalkyl and 5- or 6-membered heteroaryl,
      wherein said 4- to 7-membered heterocycloalkyl group is connected to the rest of the molecule via a carbon atom of said 4- to 7-membered heterocycloalkyl group,
      and
      wherein said 5- or 6-membered heteroaryl group is optionally substituted, one or two times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl and —N(R⁹)(R¹⁰)
R³ represents a hydrogen atom or a halogen atom or a group selected from
   C₁-C₄-alkyl, C₃-C₅-cycloalkyl, (C₁-C₂-alkoxy)-(C₁-C₄-alkyl)-, C₁-C₄-alkoxy, —S(═O)₂R¹⁴, cyano, hydroxy, —N(R⁹)(R¹⁰) and —P(═O)(R¹⁴)₂,
R⁴ represents a hydrogen atom or a halogen atom or a group selected from
   C₁-C₄-alkyl, C₃-C₅-cycloalkyl, (C₁-C₂-alkoxy)-(C₁-C₄-alkyl)-, C₁-C₄-alkoxy, (C₁-C₂-alkoxy)-(C₁-C₄-alkoxy)-, C₃-C₅-cycloalkyloxy, hydroxy, —N(R⁹)(R¹⁰), —N(R¹⁸)(R¹⁹), —P(═O)(R¹⁴)₂, 4- to 7-membered heterocycloalkyl and
   (4- to 7-membered heterocycloalkyl)oxy,
      wherein said 4- to 7-membered heterocycloalkyl group is connected to the rest of the molecule via a carbon atom of said 4- to 7-membered heterocycloalkyl group,
      and
      wherein said 4- to 7-membered heterocycloalkyl group and (4- to 7-membered heterocycloalkyl)oxy group is optionally substituted, one, two or three times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl, —N(R⁹)(R¹⁰) and oxo,
R⁵ represents a hydrogen atom or a halogen atom or a group selected from
   C₁-C₄-alkyl, C₃-C₅-cycloalkyl, C₁-C₄-alkoxy, S(═O)₂R¹⁴, cyano, —N(R⁹)(R¹⁰), —N═S(═O)(R¹⁴)₂ and —P(═O)(R¹⁴)₂,
R⁶ represents a hydrogen atom or a group selected from
   C₁-C₄-alkyl, C₁-C₄-hydroxyalkyl, C₁-C₄-alkoxy, hydroxy and oxo,
R⁷ represents a hydrogen atom or a C₁-C₄-alkyl group,
R⁸ represents a group selected from methyl and ethyl,
R⁹ and R¹⁰ represent, independently from each occurrence, a hydrogen atom or C₁-C₄-alkyl group,
   or
R⁹ and R¹⁰ together with the nitrogen to which they are attached represent a
   nitrogen containing 4- to 7-membered heterocycloalkyl group or 5- to 7-membered heterocycloalkenyl group,
      wherein said nitrogen containing 4- to 7-membered heterocycloalkyl group and 5- to 7-membered heterocycloalkenyl group are optionally substituted, one, two or three times, each substituent independently selected from a halogen atom or a group selected from
      C₁-C₄-alkyl, C₃-C₄-cycloalkyl, hydroxy and oxo,
      or
      two substituents, which are attached to the same carbon atom of said nitrogen containing 4- to 7-membered heterocycloalkyl group, together with the carbon atom to which they are attached, represent a 4- to 7-membered heterocycloalkyl group,
         wherein said 4- to 7-membered heterocycloalkyl group is optionally substituted, one or two times, each substituent independently selected from a halogen atom or a group selected from
         C₁-C₄-alkyl, C₃-C₄-cycloalkyl, C₁-C₄-haloalkyl, hydroxy and oxo,
R¹² represents a hydrogen atom or a C₁-C₄-alkyl group,
R¹³ represents a hydrogen atom or a group selected from
   C₁-C₄-alkyl, phenyl and 5- or 6-membered heteroaryl,
      wherein said phenyl group and 5- or 6-membered heteroaryl group is optionally substituted, one or two times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl and —N(R⁹)(R¹⁰),
R¹⁴ represents a C₁-C₄-alkyl group,
R¹⁵ represents a hydrogen atom or a C₁-C₄-alkyl group,
R¹⁶ represent, indepently from each other, a hydrogen atom or a C₁-C₂-alkyl group,
R¹⁷ represents a hydroxy group,
R¹⁸ represents a hydrogen atom or a C₁-C₄-alkyl group,
R¹⁹ represents a 4- to 7-membered heterocycloalkyl group,
   wherein said 4- to 7-membered heterocycloalkyl group is optionally substituted, one or two times, with a C₁-C₄-alkyl group,
   and
   wherein said 4- to 7-membered heterocycloalkyl group is connected to the rest of the molecule via a carbon atom of the 4- to 7-membered heterocycloalkyl group,
o represents an integer selected from 1 or 2,
and
p represents an integer selected from 0 and 1,
and stereoisomers, tautomers, N-oxides, hydrates, solvates, and salts thereof, and mixtures of same.
In accordance with a fourth embodiment of the first aspect, the present invention covers compounds of general formula (I), supra, in which:
n represents an integer selected from 1, 2 and 3,
X represents a group selected from
   —O—, —S—, *-(C(R¹⁶)(R¹⁷))ₒ-#, *-O—C(R¹⁶)₂—(C(R¹⁶)(R⁷))ₚ-#and *-(C(R¹⁶)(R¹⁷))ₚ—C(R¹⁶)₂—O—#,
      wherein * indicates the point of attachment to R² and #indicates the point of attachment to the pyrrolidine-, piperidine- and azepane moiety,
R¹ represents a group selected from cyano, —C(═O)NH₂, —C(═O)N(H)CH₃, —C(═O)N(CH₃)₂ and —C(═O)OR¹⁵,
R² represents a group selected from phenyl, naphthyl and 5- to 10-membered heteroaryl,
   which 5- to 10-membered heteroaryl group is connected to the rest of the molecule via a carbon atom of said 5- to 10-membered heteroaryl group,
   and
   which phenyl, naphthyl and 5- to 10-membered heteroaryl group is optionally substituted, one, two, three or four times, each substituent independently selected from a halogen atom or a group selected from
   C₁-C₄-alkyl, C₃-C₅-cycloalkyl, C₁-C₄-haloalkyl, C₁-C₄-alkoxy, (C₁-C₂-alkoxy)-(C₁-C₄-alkoxy)-, C₁-C₄-haloalkoxy, —SR¹⁴, —S(═O)R¹⁴, —S(═O)₂R¹⁴, nitro, cyano, hydroxy, —N(R⁹)(R¹⁰), —C(═O)N(R⁹)(R¹⁰), —C(═O)₂R¹⁵, —N═S(═O)(R¹⁴)₂ and 5- or 6-membered heteroaryl,
      wherein said 5- or 6-membered heteroaryl group is optionally substituted, one or two times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl and —N(R⁹)(R¹⁰),
R³ represents a hydrogen atom or a halogen atom or a group selected from
   C₁-C₄-alkyl, C₃-C₅-cycloalkyl, C₁-C₄-alkoxy, cyano and hydroxy,
R⁴ represents a hydrogen atom or a halogen atom or a group selected from
   C₁-C₄-alkyl, C₃-C₅-cycloalkyl, (C₁-C₂-alkoxy)-(C₁-C₄-alkyl)-, C₁-C₄-alkoxy, (C₁-C₂-alkoxy)-(C₁-C₄-alkoxy)-, C₃-C₅-cycloalkyloxy, hydroxy, —N(R⁹)(R¹⁰), —N(R¹⁸)(R¹⁹), —P(═O)(R¹⁴)₂, 4- to 7-membered heterocycloalkyl and (4- to 7-membered heterocycloalkyl)oxy,
      wherein said 4- to 7-membered heterocycloalkyl group is connected to the rest of the molecule via a carbon atom of said 4- to 7-membered heterocycloalkyl group,
R⁵ represents a hydrogen atom or a halogen atom or a group selected from
   S(═O)₂R¹⁴, cyano, —N(R⁹)(R¹⁰), —N═S(═O)(R¹⁴)₂ and —P(═O)(R¹⁴)₂,
R⁶ represents a hydrogen atom,
R⁷ represents a hydrogen atom or a C₁-C₄-alkyl group,
R⁸ represents a methyl group,
R⁹ and R¹⁰ represent, independently from each occurrence, a hydrogen atom or C₁-C₄-alkyl group,
   or
R⁹ and R¹⁰ together with the nitrogen to which they are attached represent a nitrogen containing 4- to 7-membered heterocycloalkyl group or 5- to 7-membered heterocycloalkenyl group,
   wherein said nitrogen containing 4- to 7-membered heterocycloalkyl group and 5- to 7-membered heterocycloalkenyl group are optionally substituted, one, two or three times, each substituent independently selected from a halogen atom or a group selected from
   C₁-C₄-alkyl and oxo,
R¹⁴ represents a C₁-C₄-alkyl group,
R¹⁵ represents C₁-C₄-alkyl group,
R¹⁶ represent, indepently from each other, a hydrogen atom or a methyl group,
R¹⁷ represents a hydroxy group,
R¹⁸ represents a hydrogen atom or a C₁-C₄-alkyl group,
R¹⁹ represents a 4- to 7-membered heterocycloalkyl group,
   wherein said 4- to 7-membered heterocycloalkyl group is optionally substituted, one or two times, with a C₁-C₄-alkyl group,
   and
   wherein said 4- to 7-membered heterocycloalkyl group is connected to the rest of the molecule via a carbon atom of the 4- to 7-membered heterocycloalkyl group,
o represents an integer of 1,
and
p represents an integer of 0,
and stereoisomers, tautomers, N-oxides, hydrates, solvates, and salts thereof, and mixtures of same.
In accordance with a fifth embodiment of the first aspect, the present invention covers compounds of general formula (I), supra, in which:
n represents an integer selected from 1, 2 and 3,
X represents a group selected from
   —O—, —S—, *-(C(R¹⁶)(R¹⁷))ₒ-#, *-O—C(R¹⁶)₂—(C(R¹⁶)(R¹⁷))ₚ-#and *-(C(R¹⁶)(R¹⁷))ₚ—C(R¹⁶)₂—O—#,
      wherein * indicates the point of attachment to R² and #indicates the point of attachment to the pyrrolidine-, piperidine- and azepane moiety,
R¹ represents a group selected from cyano, —C(═O)NH₂, —C(═O)N(H)CH₃, —C(═O)N(CH₃)₂ and —C(═O)OR¹⁵,
R² represents a group selected from phenyl, naphthyl and 5- to 10-membered heteroaryl,
   which 5- to 10-membered heteroaryl group is selected from pyridinyl, pyrimidinyl, 1,2-benzoxazolyl, 1,3-benzoxazolyl and 1,3-benzothiazolyl,
   and
   which phenyl, naphthyl and 5- to 10-membered heteroaryl group is optionally substituted, one, two, three or four times, each substituent independently selected from a halogen atom or a group selected from
   C₁-C₄-alkyl, C₃-C₅-cycloalkyl, C₁-C₄-haloalkyl, C₁-C₄-alkoxy, (C₁-C₂-alkoxy)-(C₁-C₄-alkoxy)-, C₁-C₄-haloalkoxy, —SR¹⁴, —S(═O)R¹⁴, —S(═O)₂R¹⁴, nitro, cyano, hydroxy, —N(R⁹)(R¹⁰), —C(═O)N(R⁹)(R¹⁰), —C(═O)₂R¹⁵, —N═S(═O)(R¹⁴)₂ and 5- or 6-membered heteroaryl,
      wherein said 5- or 6-membered heteroaryl group is optionally substituted, one or two times, each substituent independently selected from a halogen atom or a C₁-C₂-alkyl group,
R³ represents a hydrogen atom or a halogen atom or a group selected from
   C₁-C₄-alkyl, C₃-C₅-cycloalkyl, C₁-C₄-alkoxy, cyano and hydroxy,
R⁴ represents a hydrogen atom or a halogen atom or a group selected from
   C₁-C₄-alkyl, C₃-C₅-cycloalkyl, (C₁-C₂-alkoxy)-(C₁-C₄-alkyl)-, C₁-C₄-alkoxy, (C₁-C₂-alkoxy)-(C₁-C₄-alkoxy)-, C₃-C₅-cycloalkyloxy, hydroxy, —N(R⁹)(R¹⁰), —N(R¹⁸)(R¹⁹), —P(═O)(R¹⁴)₂, 4- to 7-membered heterocycloalkyl and (4- to 7-membered heterocycloalkyl)oxy,
      wherein said 4- to 7-membered heterocycloalkyl group is connected to the rest of the molecule via a carbon atom of said 4- to 7-membered heterocycloalkyl group,
R⁵ represents a hydrogen atom or a halogen atom or a group selected from
   S(═O)₂R¹⁴, cyano, —N(R⁹)(R¹⁰), —N═S(═O)(R¹⁴)₂ and —P(═O)(R¹⁴)₂,
R⁶ represents a hydrogen atom,
R⁷ represents a hydrogen atom or C₁-C₄-alkyl group,
R⁸ represents a methyl group,
R⁹ and R¹⁰ represent, independently from each occurrence, a hydrogen atom or C₁-C₄-alkyl group,
   or
R⁹ and R¹⁰ together with the nitrogen to which they are attached represent a nitrogen containing 4- to 7-membered heterocycloalkyl group or
   5- to 7-membered heterocycloalkenyl group,
      wherein said nitrogen containing 4- to 7-membered heterocycloalkyl group and 5- to 7-membered heterocycloalkenyl group are optionally substituted, one, two or three times, each substituent independently selected from a halogen atom or a group selected from
      C₁-C₄-alkyl and oxo,
R¹⁴ represents a C₁-C₄-alkyl group,
R¹⁵ represents C₁-C₄-alkyl group,
R¹⁶ represent, indepently from each other, a hydrogen atom or a methyl group,
R¹⁷ represents a hydroxy group,
R¹⁸ represents a hydrogen atom or a C₁-C₄-alkyl group,
R¹⁹ represents a 4- to 7-membered heterocycloalkyl group,
   wherein said 4- to 7-membered heterocycloalkyl group is connected to the rest of the molecule via a carbon atom of the 4- to 7-membered heterocycloalkyl group,
o represents an integer of 1,
and
p represents an integer of 0,
and stereoisomers, tautomers, N-oxides, hydrates, solvates, and salts thereof, and mixtures of same.
In accordance with a sixth embodiment of the first aspect, the present invention covers compounds of general formula (I), supra, in which:
n represents an integer selected from 1, 2 and 3,
X represents a group selected from
   —O—, —S—, —CH₂—, —CH(OH)—, *—OCH₂-#, *-CH₂O—#and *-CH(CH₃)O-#,
      wherein * indicates the point of attachment to R² and #indicates the point of attachment to the pyrrolidine-, piperidine- and azepane moiety,
R¹ represents a group selected from cyano and —C(═O)NH₂,
R² represents a group selected from phenyl, pyridin-2-yl, pyridin-3-yl, pyrimidin-5-yl, 1,2-benzoxazol-6-yl, 1,3-benzoxazol-4-yl and 1,3-benzothiazol-2-yl,
   which group is optionally substituted, one or two times, each substituent independently selected from a fluorine, chlorine, or bromine atom or a group selected from
   methyl, cyclopropyl, difluoromethyl, trifluoromethyl, methoxy, ethoxy, isopropoxy, 2-methoxyethoxy, difluoromethoxy, trifluormethoxy, methylsulfanyl, methylsulfinyl, methylsulfonyl, nitro, cyano, amino, dimethylamino, azetidin-1-yl, 2-oxopyrrolidin-1-yl, 3-methyl-2-oxo-2,3-dihydro-1H-imidazol-1-yl, 2-oxopiperidin-1-yl, 4-methyl-2-oxopiperazin-1-yl, morpholino-4-yl, 3-methyl-2-oxoimidazolidin-1-yl, 3-methyl-2-oxo-1,3-diazinan-1-yl, carbamoyl, dimethylcarbamoyl, ethoxycarbonyl, [dimethyl(oxido)-Δ⁶-sulfanylidene]amino, 1H-imidazol-1-yl, 1-methyl-1H-pyrazol-4-yl and 1H-1,2,4-triazol-1-yl,
R³ represents a hydrogen atom or a fluorine, chlorine or bromine atom or a group selected from
   methyl, cyclopropyl, methoxy, cyano and hydroxy,
R⁴ represents a hydrogen atom or a fluorine, chlorine or bromine atom or a group selected from
   methyl, cyclopropyl, 2-methoxyethyl, methoxy, propoxy, 2-methoxyethoxy, cyclopropyloxy, hydroxy, 2-oxopyrrolidin-1-yl, 4-methylpiperazin-1-yl, methyl(tetrahydrofuran-3-yl)amino, dimethylphosphoryl, oxetan-3-yl, (oxetan-3-yl)oxy, tetrahydrofuranyl-3-oxy, (tetrahydro-2H-pyran-3-yl)oxy and (tetrahydro-2H-pyran-4-yl)oxy,
R⁵ represents a hydrogen atom or a fluorine, chlorine or bromine atom or a group selected from
   methanesulfonyl, cyano, 2-oxopyrrolidin-1-yl, [dimethyl(oxido)-Δ⁶-sulfanylidene]amino and dimethylphosphoryl,
R⁶ represents a hydrogen atom,
R⁷ represents a hydrogen atom or a methyl group,
and
R⁸ represents a methyl group,
and stereoisomers, tautomers, N-oxides, hydrates, solvates, and salts thereof, and mixtures of same.
   In a further embodiment of the first aspect, the present invention covers compounds of formula (I), supra, in which:
   X represents a group selected from
   —O—, —S—, —S(═O)—, —S(═O)₂—, *-(C(R¹⁶)(R¹⁷))ₒ-#, *—O—C(R¹⁶)₂—(C(R¹⁶)(R¹⁷))ₚ-#, *-(C(R¹⁶)(R¹⁷))ₚ—C(R¹⁶)₂—O—#,

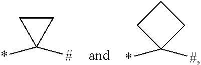

wherein * indicates the point of attachment to R² and #indicates the point of attachment to the pyrrolidine-, piperidine- and azepane moiety,
and stereoisomers, tautomers, N-oxides, hydrates, solvates, and salts thereof, and mixtures of same.

In a further embodiment of the first aspect, the present invention covers compounds of formula (I), supra, in which:
X represents a group selected from
   —O—, —S—, *-(C(R¹⁶)(R¹⁷))ₒ-#, *—O—C(R¹⁶)₂—(C(R¹⁶)(R¹⁷))ₚ-#and *-(C(R¹⁶)(R¹⁷))ₚ—C(R¹⁶)₂—O—#,
      wherein * indicates the point of attachment to R² and #indicates the point of attachment to the pyrrolidine-, piperidine- and azepane moiety,
and stereoisomers, tautomers, N-oxides, hydrates, solvates, and salts thereof, and mixtures of same.

In a further embodiment of the first aspect, the present invention covers compounds of formula (I), supra, in which:
X represents a group selected from
   —O—, —S—, —CH₂—, —CH(OH)—, *—OCH₂-#, *-CH₂O—#and *-CH(CH₃)O-#,
      wherein * indicates the point of attachment to R² and #indicates the point of attachment to the pyrrolidine-, piperidine- and azepane moiety,
and stereoisomers, tautomers, N-oxides, hydrates, solvates, and salts thereof, and mixtures of same.

In a further embodiment of the first aspect, the present invention covers compounds of formula (I), supra, in which:
R¹ represents a group selected from cyano, —C(═O)NH₂, —C(═O)N(H)CH₃, —C(═O)N(H)C₂H₅, —C(═O)N(CH₃)₂ and —C(═O)OR¹⁵,
and stereoisomers, tautomers, N-oxides, hydrates, solvates, and salts thereof, and mixtures of same.
In a further embodiment of the first aspect, the present invention covers compounds of formula (I), supra, in which:
R¹ represents a group selected from cyano, —C(═O)NH₂, —C(═O)N(H)CH₃, —C(═O)N(CH₃)₂ and —C(═O)OR¹⁵,
and stereoisomers, tautomers, N-oxides, hydrates, solvates, and salts thereof, and mixtures of same.

In a further embodiment of the first aspect, the present invention covers compounds of formula (I), supra, in which:
R¹ represents a group selected from cyano, —C(═O)NH₂, —C(═O)N(H)CH₃ and —C(═O)N(CH₃)₂,
and stereoisomers, tautomers, N-oxides, hydrates, solvates, and salts thereof, and mixtures of same.

In a further embodiment of the first aspect, the present invention covers compounds of formula (I), supra, in which:
R¹ represents a group selected from —C(═O)NH₂, —C(═O)N(H)CH₃ and —C(═O)N(CH₃)₂,
and stereoisomers, tautomers, N-oxides, hydrates, solvates, and salts thereof, and mixtures of same.

In a further embodiment of the first aspect, the present invention covers compounds of formula (I), supra, in which:
R¹ represents a group selected from cyano and —C(═O)NH₂,
and stereoisomers, tautomers, N-oxides, hydrates, solvates, and salts thereof, and mixtures of same.

In a further embodiment of the first aspect, the present invention covers compounds of formula (I), supra, in which:
R¹ represents a cyano group,
and stereoisomers, tautomers, N-oxides, hydrates, solvates, and salts thereof, and mixtures of same.

In a further embodiment of the first aspect, the present invention covers compounds of formula (I), supra, in which:
R¹ represents a —C(═O)NH₂ group,
and stereoisomers, tautomers, N-oxides, hydrates, solvates, and salts thereof, and mixtures of same.
In a further embodiment of the first aspect, the present invention covers compounds of formula (I), supra, in which:
R² represents a group selected from phenyl, naphthyl and 5- to 10-membered heteroaryl,
   which 5- to 10-membered heteroaryl group is connected to the rest of the molecule via a carbon atom of said 5- to 10-membered heteroaryl group,
   and
   which phenyl, naphthyl and 5- to 10-membered heteroaryl group is optionally substituted, one, two, three or four times, each substituent independently selected from a halogen atom or a group selected from
   C₁-C₆-alkyl, C₃-C₆-cycloalkyl, C₁-C₆-hydroxyalkyl, C₁-C₆-haloalkyl, (C₁-C₂-alkoxy)-(C₁-C₆-alkyl)-, C₁-C₆-alkoxy, (C₁-C₂-alkoxy)-(C₁-C₆-alkoxy)-, C₁-C₆-haloalkoxy, C₃-C₆-cycloalkyloxy, phenoxy, —SR¹⁴, —S(═O)R¹⁴, —S(═O)₂R¹⁴, —P(═O)(R¹⁴)₂, nitro, cyano, hydroxy, —N(R⁹)(R¹⁰), —C(═O)N(R⁹)(R¹⁰), —C(═O)R¹¹, —C(═O)₂R¹⁵, —N(R¹²)C(═O)R¹³, —N(R¹²)S(═O)₂R¹⁴, —N═S(═NH)(R¹⁴)₂, —N═S(═O)(R¹⁴)₂, —S(═O)(═NR¹⁴)R¹³, 4- to 7-membered heterocycloalkyl, 5- to 7-membered heterocycloalkenyl, (4- to 7-membered heterocycloalkyl)oxy, phenyl and 5- or 6-membered heteroaryl,
   or two substituents of said phenyl group, when they are attached to adjacent ring atoms, are optionally linked to one another in such a way that they jointly form a group selected from
   —(CH₂)₃—, —(CH₂)₄—, —O—(CH₂)₂—, —(CH₂)₂—O—, —CH₂—O—CH₂—, —O—(CH₂)₃—, —(CH₂)₃—O—, —CH₂—O—(CH₂)₂—, —(CH₂)₂O—CH₂—, —O—CH₂—O— and —O—(CH₂)₂—O—,
      wherein said 4- to 7-membered heterocycloalkyl group and
      5- to 7-membered heterocycloalkenyl group is connected to the rest of the molecule via a carbon atom of said
      4- to 7-membered heterocycloalkyl group and
      5- to 7-membered heterocycloalkenyl group,
      and
      wherein said 4- to 7-membered heterocycloalkyl group,
      5- to 7-membered heterocycloalkenyl group and
      (4- to 7-membered heterocycloalkyl)oxy group is optionally substituted, one, two or three times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl, —N(R⁹)(R¹⁰) and oxo,
      and
      wherein said C₁-C₆-alkyl and C₁-C₆-alkoxy group is optionally substituted with a group selected from
      C₃-C₄-cycloalkyl, phenyl and 4- to 7-membered heterocycloalkyl,
         wherein said 4- to 7-membered heterocycloalkyl group is connected to the rest of the molecule via a carbon atom of said 4- to 7-membered heterocycloalkyl group,
         and
         wherein said 4- to 7-membered heterocycloalkyl group is optionally substituted, one, two or three times, each substituent independently selected from a halogen atom or a group selected from
         C₁-C₂-alkyl, C₁-C₂-haloalkyl, cyano, hydroxy, C₁-C₂-alkoxy, C₃-C₄-cycloalkyl, —N(R⁹)(R¹⁰) and oxo,
         and
         which phenyl group is optionally substituted, one or two times, each substituent independently selected from a halogen atom or a group selected from
         C₁-C₂-alkyl, C₁-C₂-haloalkyl, cyano, hydroxy, C₁-C₂-alkoxy, C₃-C₄-cycloalkyl and —N(R⁹)(R¹⁰),
         and
         which C₃-C₄-cycloalkyl group is optionally substituted, one or two times, each substituent independently selected from a halogen atom or a group selected from cyano and hydroxy,
      and
      wherein said C₃-C₆-cycloalkyl group is optionally substituted, one or two times, each substituent independently selected from a halogen atom or a C₁-C₄-alkyl group,
      and
      wherein said phenyl, phenoxy and 5- or 6-membered heteroaryl group is optionally substituted, one or two times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl and —N(R⁹)(R¹⁰)
and stereoisomers, tautomers, N-oxides, hydrates, solvates, and salts thereof, and mixtures of same.
In a further embodiment of the first aspect, the present invention covers compounds of formula (I), supra, in which:
R² represents a phenyl group,
   which group is optionally substituted, one, two, three or four times, each substituent independently selected from a halogen atom or a group selected from
   C₁-C₆-alkyl, C₃-C₆-cycloalkyl, C₁-C₆-hydroxyalkyl, C₁-C₆-haloalkyl, (C₁-C₂-alkoxy)-(C₁-C₆-alkyl)-, C₁-C₆-alkoxy, (C₁-C₂-alkoxy)-(C₁-C₆-alkoxy)-, C₁-C₆-haloalkoxy, C₃-C₆-cycloalkyloxy, phenoxy, —SR¹⁴, —S(═O)R¹⁴, —S(═O)₂R¹⁴, —P(═O)(R¹⁴)₂, nitro, cyano, hydroxy, —N(R⁹)(R¹⁰), —C(═O)N(R⁹)(R¹⁰), —C(═O)R¹¹, —C(═O)₂R¹⁵, —N(R¹²)C(═O)R¹³, —N(R¹²)S(═O)₂R¹⁴, —N═S(═NH)(R¹⁴)₂, —N═S(═O)(R¹⁴)₂, —S(═O)(═NR¹⁴)R¹³, 4- to 7-membered heterocycloalkyl, 5- to 7-membered heterocycloalkenyl, (4- to 7-membered heterocycloalkyl)oxy, phenyl and 5- or 6-membered heteroaryl,
   or two substituents of said phenyl group, when they are attached to adjacent ring atoms, are optionally linked to one another in such a way that they jointly form a group selected from
   —(CH₂)₃—, —(CH₂)₄—, —O—(CH₂)₂—, —(CH₂)₂—O—, —CH₂—O—CH₂—, —O—(CH₂)₃—, —(CH₂)₃—O—, —CH₂—O—(CH₂)₂—, —(CH₂)₂O—CH₂—, —O—CH₂—O— and —O—(CH₂)₂—O—,
      wherein said 4- to 7-membered heterocycloalkyl group and
      5- to 7-membered heterocycloalkenyl group is connected to the rest of the molecule via a carbon atom of said
      4- to 7-membered heterocycloalkyl group and
      5- to 7-membered heterocycloalkenyl group,
      and
      wherein said 4- to 7-membered heterocycloalkyl group,
      5- to 7-membered heterocycloalkenyl group and
      (4- to 7-membered heterocycloalkyl)oxy group is optionally substituted, one, two or three times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl, —N(R⁹)(R¹⁰) and oxo,
      and
      wherein said C₁-C₆-alkyl and C₁-C₆-alkoxy group is optionally substituted with a group selected from
      C₃-C₄-cycloalkyl, phenyl and 4- to 7-membered heterocycloalkyl,
         wherein said 4- to 7-membered heterocycloalkyl group is connected to the rest of the molecule via a carbon atom of said 4- to 7-membered heterocycloalkyl group,
         and
         wherein said 4- to 7-membered heterocycloalkyl group is optionally substituted, one, two or three times, each substituent independently selected from a halogen atom or a group selected from
         C₁-C₂-alkyl, C₁-C₂-haloalkyl, cyano, hydroxy, C₁-C₂-alkoxy, C₃-C₄-cycloalkyl, —N(R⁹)(R¹⁰) and oxo,
         and
         which phenyl group is optionally substituted, one or two times, each substituent independently selected from a halogen atom or a group selected from
         C₁-C₂-alkyl, C₁-C₂-haloalkyl, cyano, hydroxy, C₁-C₂-alkoxy, C₃-C₄-cycloalkyl and —N(R⁹)(R¹⁰),
         and
         which C₃-C₄-cycloalkyl group is optionally substituted, one or two times, each substituent independently selected from a halogen atom or a group selected from cyano and hydroxy,
      and
      wherein said C₃-C₆-cycloalkyl group is optionally substituted, one or two times, each substituent independently selected from
      a halogen atom or a C₁-C₄-alkyl group,
      and
      wherein said phenyl, phenoxy and 5- or 6-membered heteroaryl group is optionally substituted, one or two times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl and —N(R⁹)(R¹⁰)
and stereoisomers, tautomers, N-oxides, hydrates, solvates, and salts thereof, and mixtures of same.
In a further embodiment of the first aspect, the present invention covers compounds of formula (I), supra, in which:
R² represents a 5- to 10-membered heteroaryl group,
   which group is connected to the rest of the molecule via a carbon atom of said group,
   and
   which group is optionally substituted, one, two, three or four times, each substituent independently selected from a halogen atom or a group selected from
   C₁-C₆-alkyl, C₃-C₆-cycloalkyl, C₁-C₆-hydroxyalkyl, C₁-C₆-haloalkyl, (C₁-C₂-alkoxy)-(C₁-C₆-alkyl)-, C₁-C₆-alkoxy, (C₁-C₂-alkoxy)-(C₁-C₆-alkoxy)-, C₁-C₆-haloalkoxy, C₃-C₆-cycloalkyloxy, phenoxy, —SR¹⁴, —S(═O)R¹⁴, —S(═O)₂R¹⁴, —P(═O)(R¹⁴)₂, nitro, cyano, hydroxy, —N(R⁹)(R¹⁰), —C(═O)N(R⁹)(R¹⁰), —C(═O)R¹¹, —C(═O)₂R¹⁵, —N(R¹²)C(═O)R¹³, —N(R¹²)S(═O)₂R¹⁴, —N═S(═NH)(R¹⁴)₂, —N═S(═O)(R¹⁴)₂, —S(═O)(═NR¹⁴)R¹³, 4- to 7-membered heterocycloalkyl, 5- to 7-membered heterocycloalkenyl, (4- to 7-membered heterocycloalkyl)oxy, phenyl and 5- or 6-membered heteroaryl,
   or two substituents of said phenyl group, when they are attached to adjacent ring atoms, are optionally linked to one another in such a way that they jointly form a group selected from
   —(CH₂)₃—, —(CH₂)₄—, —O—(CH₂)₂—, —(CH₂)₂—O—, —CH₂—O—CH₂—, —O—(CH₂)₃—, —(CH₂)₃—O—, —CH₂—O—(CH₂)₂—, —(CH₂)₂O—CH₂—, —O—CH₂—O— and —O—(CH₂)₂—O—,
      wherein said 4- to 7-membered heterocycloalkyl group and
      5- to 7-membered heterocycloalkenyl group is connected to the rest of the molecule via a carbon atom of said
      4- to 7-membered heterocycloalkyl group and
      5- to 7-membered heterocycloalkenyl group,
      and
      wherein said 4- to 7-membered heterocycloalkyl group, 5- to 7-membered heterocycloalkenyl group and (4- to 7-membered heterocycloalkyl)oxy group is optionally substituted, one, two or three times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl, —N(R⁹)(R¹⁰) and oxo,
      and
      wherein said C₁-C₆-alkyl and C₁-C₆-alkoxy group is optionally substituted with a group selected from
      C₃-C₄-cycloalkyl, phenyl and 4- to 7-membered heterocycloalkyl,
         wherein said 4- to 7-membered heterocycloalkyl group is connected to the rest of the molecule via a carbon atom of said 4- to 7-membered heterocycloalkyl group,
         and
         wherein said 4- to 7-membered heterocycloalkyl group is optionally substituted, one, two or three times, each substituent independently selected from a halogen atom or a group selected from
         C₁-C₂-alkyl, C₁-C₂-haloalkyl, cyano, hydroxy, C₁-C₂-alkoxy, C₃-C₄-cycloalkyl, —N(R⁹)(R¹⁰) and oxo,
         and
         which phenyl group is optionally substituted, one or two times, each substituent independently selected from a halogen atom or a group selected from
         C₁-C₂-alkyl, C₁-C₂-haloalkyl, cyano, hydroxy, C₁-C₂-alkoxy, C₃-C₄-cycloalkyl and —N(R⁹)(R¹⁰),
         and
         which C₃-C₄-cycloalkyl group is optionally substituted, one or two times, each substituent independently selected from a halogen atom or a group selected from
         cyano and hydroxy,
      and
      wherein said C₃-C₆-cycloalkyl group is optionally substituted, one or two times, each substituent independently selected from a halogen atom or a C₁-C₄-alkyl group,
      and
      wherein said phenyl, phenoxy and 5- or 6-membered heteroaryl group is optionally substituted, one or two times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl and —N(R⁹)(R¹⁰)
In a further embodiment of the first aspect, the present invention covers compounds of formula (I), supra, in which:
R² represents a group selected from phenyl, naphthyl and 5- to 10-membered heteroaryl,
   which 5- to 10-membered heteroaryl group is connected to the rest of the molecule via a carbon atom of said 5- to 10-membered heteroaryl group,
   and
   which phenyl, naphthyl and 5- to 10-membered heteroaryl group is optionally substituted, one, two, three or four times, each substituent independently selected from a halogen atom or a group selected from
   C₁-C₄-alkyl, C₃-C₅-cycloalkyl, C₁-C₄-hydroxyalkyl, C₁-C₄-haloalkyl, (C₁-C₂-alkoxy)-(C₁-C₄-alkyl)-, C₁-C₄-alkoxy, (C₁-C₂-alkoxy)-(C₁-C₄-alkoxy)-, C₁-C₄-haloalkoxy, C₃-C₅-cycloalkyloxy, phenoxy, —SR¹⁴, —S(═O)R¹⁴, —S(═O)₂R¹⁴, —P(═O)(R¹⁴)₂, nitro, cyano, hydroxy, —N(R⁹)(R¹⁰), —C(═O)N(R⁹)(R¹⁰), —C(═O)R¹¹, —C(═O)₂R¹⁵, —N(R¹²)C(═O)R¹³, —N(R¹²)S(═O)₂R¹⁴, —N═S(═NH)(R¹⁴)₂, —N═S(═O)(R¹⁴)₂, —S(═O)(═NR¹⁴)R¹³, 4- to 7-membered heterocycloalkyl, 5- to 7-membered heterocycloalkenyl, (4- to 7-membered heterocycloalkyl)oxy, phenyl and 5- or 6-membered heteroaryl,
   or two substituents of said phenyl group, when they are attached to adjacent ring atoms, are optionally linked to one another in such a way that they jointly form a group selected from
   —(CH₂)₃—, —(CH₂)₄—, —O—(CH₂)₂—, —(CH₂)₂—O—, —CH₂—O—CH₂—, —O—(CH₂)₃—, —(CH₂)₃—O—, —CH₂—O—(CH₂)₂—, —(CH₂)₂O—CH₂—, —O—CH₂—O— and —O—(CH₂)₂—O—,
      wherein said 4- to 7-membered heterocycloalkyl group and
      5- to 7-membered heterocycloalkenyl group is connected to the rest of the molecule via a carbon atom of said
      4- to 7-membered heterocycloalkyl group and
      5- to 7-membered heterocycloalkenyl group,
      and
      wherein said 4- to 7-membered heterocycloalkyl group,
      5- to 7-membered heterocycloalkenyl group and
      (4- to 7-membered heterocycloalkyl)oxy group is optionally substituted, one, two or three times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl, —N(R⁹)(R¹⁰) and oxo,
      and
      wherein said C₁-C₄-alkyl and C₁-C₄-alkoxy group is optionally substituted with a group selected from
      C₃-C₄-cycloalkyl, phenyl and 4- to 7-membered heterocycloalkyl,
         wherein said 4- to 7-membered heterocycloalkyl group is connected to the rest of the molecule via a carbon atom of said 4- to 7-membered heterocycloalkyl group,
         and
         wherein said 4- to 7-membered heterocycloalkyl group is optionally substituted, one, two or three times, each substituent independently selected from a halogen atom or a group selected from
         C₁-C₂-alkyl, C₁-C₂-haloalkyl, cyano, hydroxy, C₁-C₂-alkoxy, C₃-C₄-cycloalkyl, —N(R⁹)(R¹⁰) and oxo,
         and
         which phenyl group is optionally substituted, one or two times, each substituent independently selected from a halogen atom or a group selected from
         C₁-C₂-alkyl, C₁-C₂-haloalkyl, cyano, hydroxy, C₁-C₂-alkoxy, C₃-C₄-cycloalkyl and —N(R⁹)(R¹⁰),
         and
         which C₃-C₄-cycloalkyl group is optionally substituted, one or two times, each substituent independently selected from a halogen atom or a group selected from
         cyano and hydroxy,
      and
      wherein said C₃-C₅-cycloalkyl group is optionally substituted, one or two times, each substituent independently selected from
      a halogen atom or a C₁-C₄-alkyl group,
      and
      wherein said phenyl, phenoxy and 5- or 6-membered heteroaryl group is optionally substituted, one or two times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl and —N(R⁹)(R¹⁰)
and stereoisomers, tautomers, N-oxides, hydrates, solvates, and salts thereof, and mixtures of same.
In a further embodiment of the first aspect, the present invention covers compounds of formula (I), supra, in which:
R² represents a phenyl group,
   which group is optionally substituted, one, two, three or four times, each substituent independently selected from a halogen atom or a group selected from
   C₁-C₄-alkyl, C₃-C₅-cycloalkyl, C₁-C₄-hydroxyalkyl, C₁-C₄-haloalkyl, (C₁-C₂-alkoxy)-(C₁-C₄-alkyl)-, C₁-C₄-alkoxy, (C₁-C₂-alkoxy)-(C₁-C₄-alkoxy)-, C₁-C₄-haloalkoxy, C₃-C₅-cycloalkyloxy, phenoxy, —SR¹⁴, —S(═O)R¹⁴, —S(═O)₂R¹⁴, —P(═O)(R¹⁴)₂, nitro, cyano, hydroxy, —N(R⁹)(R¹⁰), —C(═O)N(R⁹)(R¹⁰), —C(═O)R¹¹, —C(═O)₂R¹⁵, —N(R¹²)C(═O)R¹³, —N(R¹²)S(═O)₂R¹⁴, —N═S(═NH)(R¹⁴)₂, —N═S(═O)(R¹⁴)₂, —S(═O)(═NR¹⁴)R¹³, 4- to 7-membered heterocycloalkyl, 5- to 7-membered heterocycloalkenyl, (4- to 7-membered heterocycloalkyl)oxy, phenyl and 5- or 6-membered heteroaryl,
   or two substituents of said phenyl group, when they are attached to adjacent ring atoms, are optionally linked to one another in such a way that they jointly form a group selected from
   —(CH₂)₃—, —(CH₂)₄—, —O—(CH₂)₂—, —(CH₂)₂—O—, —CH₂—O—CH₂—, —O—(CH₂)₃—, —(CH₂)₃—O—, —CH₂—O—(CH₂)₂—, —(CH₂)₂O—CH₂—, —O—CH₂—O— and —O—(CH₂)₂—O—,
      wherein said 4- to 7-membered heterocycloalkyl group and
      5- to 7-membered heterocycloalkenyl group is connected to the rest of the molecule via a carbon atom of said
      4- to 7-membered heterocycloalkyl group and
      5- to 7-membered heterocycloalkenyl group,
      and
      wherein said 4- to 7-membered heterocycloalkyl group,
      5- to 7-membered heterocycloalkenyl group and
      (4- to 7-membered heterocycloalkyl)oxy group is optionally substituted, one, two or three times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl, —N(R⁹)(R¹⁰) and oxo,
      and
      wherein said C₁-C₄-alkyl and C₁-C₄-alkoxy group is optionally substituted with a group selected from
      C₃-C₄-cycloalkyl, phenyl and 4- to 7-membered heterocycloalkyl,
         wherein said 4- to 7-membered heterocycloalkyl group is connected to the rest of the molecule via a carbon atom of said 4- to 7-membered heterocycloalkyl group,
         and
         wherein said 4- to 7-membered heterocycloalkyl group is optionally substituted, one, two or three times, each substituent independently selected from a halogen atom or a group selected from
         C₁-C₂-alkyl, C₁-C₂-haloalkyl, cyano, hydroxy, C₁-C₂-alkoxy, C₃-C₄-cycloalkyl, —N(R⁹)(R¹⁰) and oxo,
         and
         which phenyl group is optionally substituted, one or two times, each substituent independently selected from a halogen atom or a group selected from
         C₁-C₂-alkyl, C₁-C₂-haloalkyl, cyano, hydroxy, C₁-C₂-alkoxy, C₃-C₄-cycloalkyl and —N(R⁹)(R¹⁰),
         and
         which C₃-C₄-cycloalkyl group is optionally substituted, one or two times, each substituent independently selected from a halogen atom or a group selected from cyano and hydroxy,
      and
      wherein said C₃-C₅-cycloalkyl group is optionally substituted, one or two times, each substituent independently selected from
      a halogen atom or a C₁-C₄-alkyl group,
      and
      wherein said phenyl, phenoxy and 5- or 6-membered heteroaryl group is optionally substituted, one or two times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl and —N(R⁹)(R¹⁰)
and stereoisomers, tautomers, N-oxides, hydrates, solvates, and salts thereof, and mixtures of same.
In a further embodiment of the first aspect, the present invention covers compounds of formula (I), supra, in which:
R² represents a 5- to 10-membered heteroaryl,
   which group is connected to the rest of the molecule via a carbon atom of said group,
   and
   which group is optionally substituted, one, two, three or four times, each substituent independently selected from a halogen atom or a group selected from
   C₁-C₄-alkyl, C₃-C₅-cycloalkyl, C₁-C₄-hydroxyalkyl, C₁-C₄-haloalkyl, (C₁-C₂-alkoxy)-(C₁-C₄-alkyl)-, C₁-C₄-alkoxy, (C₁-C₂-alkoxy)-(C₁-C₄-alkoxy)-, C₁-C₄-haloalkoxy, C₃-C₅-cycloalkyloxy, phenoxy, —SR¹⁴, —S(═O)R¹⁴, —S(═O)₂R¹⁴, —P(═O)(R¹⁴)₂, nitro, cyano, hydroxy, —N(R⁹)(R¹⁰), —C(═O)N(R⁹)(R¹⁰), —C(═O)R¹¹, —C(═O)₂R¹⁵, —N(R¹²)C(═O)R¹³, —N(R¹²)S(═O)₂R¹⁴, —N═S(═NH)(R¹⁴)₂, —N═S(═O)(R¹⁴)₂, —S(═O)(═NR¹⁴)R¹³, 4- to 7-membered heterocycloalkyl, 5- to 7-membered heterocycloalkenyl, (4- to 7-membered heterocycloalkyl)oxy, phenyl and 5- or 6-membered heteroaryl,
   or two substituents of said phenyl group, when they are attached to adjacent ring atoms, are optionally linked to one another in such a way that they jointly form a group selected from
   —(CH₂)₃—, —(CH₂)₄—, —O—(CH₂)₂—, —(CH₂)₂—O—, —CH₂—O—CH₂—, —O—(CH₂)₃—, —(CH₂)₃—O—, —CH₂—O—(CH₂)₂—, —(CH₂)₂O—CH₂—, —O—CH₂—O— and —O—(CH₂)₂—O—,
      wherein said 4- to 7-membered heterocycloalkyl group and
      5- to 7-membered heterocycloalkenyl group is connected to the rest of the molecule via a carbon atom of said
      4- to 7-membered heterocycloalkyl group and
      5- to 7-membered heterocycloalkenyl group,
      and
      wherein said 4- to 7-membered heterocycloalkyl group,
      5- to 7-membered heterocycloalkenyl group and
      (4- to 7-membered heterocycloalkyl)oxy group is optionally substituted, one, two or three times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl, —N(R⁹)(R¹⁰) and oxo,
      and
      wherein said C₁-C₄-alkyl and C₁-C₄-alkoxy group is optionally substituted with a group selected from
      C₃-C₄-cycloalkyl, phenyl and 4- to 7-membered heterocycloalkyl,
         wherein said 4- to 7-membered heterocycloalkyl group is connected to the rest of the molecule via a carbon atom of said 4- to 7-membered heterocycloalkyl group,
         and
         wherein said 4- to 7-membered heterocycloalkyl group is optionally substituted, one, two or three times, each substituent independently selected from a halogen atom or a group selected from
         C₁-C₂-alkyl, C₁-C₂-haloalkyl, cyano, hydroxy, C₁-C₂-alkoxy, C₃-C₄-cycloalkyl, —N(R⁹)(R¹⁰) and oxo,
         and
         which phenyl group is optionally substituted, one or two times, each substituent independently selected from a halogen atom or a group selected from
         C₁-C₂-alkyl, C₁-C₂-haloalkyl, cyano, hydroxy, C₁-C₂-alkoxy, C₃-C₄-cycloalkyl and —N(R⁹)(R¹⁰),
         and
         which C₃-C₄-cycloalkyl group is optionally substituted, one or two times, each substituent independently selected from a halogen atom or a group selected from cyano and hydroxy,
      and
      wherein said C₃-C₅-cycloalkyl group is optionally substituted, one or two times, each substituent independently selected from a halogen atom or a C₁-C₄-alkyl group,
      and
      wherein said phenyl, phenoxy and 5- or 6-membered heteroaryl group is optionally substituted, one or two times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl and —N(R⁹)(R¹⁰)
and stereoisomers, tautomers, N-oxides, hydrates, solvates, and salts thereof, and mixtures of same.
In a further embodiment of the first aspect, the present invention covers compounds of formula (I), supra, in which:
R² represents a group selected from phenyl, naphthyl and 5- to 10-membered heteroaryl,
   which 5- to 10-membered heteroaryl group is connected to the rest of the molecule via a carbon atom of said 5- to 10-membered heteroaryl group,
   and
   which phenyl, naphthyl and 5- to 10-membered heteroaryl group is optionally substituted, one, two, three or four times, each substituent independently selected from a halogen atom or a group selected from
   C₁-C₄-alkyl, C₃-C₅-cycloalkyl, C₁-C₄-haloalkyl, C₁-C₄-alkoxy, (C₁-C₂-alkoxy)-(C₁-C₄-alkoxy)-, C₁-C₄-haloalkoxy, —SR¹⁴, —S(═O)R¹⁴, —S(═O)₂R¹⁴, —P(═O)(R¹⁴)₂, nitro, cyano, hydroxy, —N(R⁹)(R¹⁰), —C(═O)N(R⁹)(R¹⁰), —C(═O)₂R¹⁵, —N(R¹²)S(═O)₂R¹⁴, —N═S(═O)(R¹⁴)₂, —S(═O)(═NR¹⁴)R¹³,
   4- to 7-membered heterocycloalkyl and 5- or 6-membered heteroaryl,
      wherein said 4- to 7-membered heterocycloalkyl group is connected to the rest of the molecule via a carbon atom of said 4- to 7-membered heterocycloalkyl group,
      and
      wherein said 5- or 6-membered heteroaryl group is optionally substituted, one or two times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl and —N(R⁹)(R¹⁰),
and stereoisomers, tautomers, N-oxides, hydrates, solvates, and salts thereof, and mixtures of same.

In a further embodiment of the first aspect, the present invention covers compounds of formula (I), supra, in which:
R² represents a phenyl group,
   which group is optionally substituted, one, two, three or four times, each substituent independently selected from a halogen atom or a group selected from
   C₁-C₄-alkyl, C₃-C₅-cycloalkyl, C₁-C₄-haloalkyl, C₁-C₄-alkoxy, (C₁-C₂-alkoxy)-(C₁-C₄-alkoxy)-, C₁-C₄-haloalkoxy, —SR¹⁴, —S(═O)R¹⁴, —S(═O)₂R¹⁴, —P(═O)(R¹⁴)₂, nitro, cyano, hydroxy, —N(R⁹)(R¹⁰), —C(═O)N(R⁹)(R¹⁰), —C(═O)₂R¹⁵, —N(R¹²)S(═O)₂R¹⁴, —N═S(═O)(R¹⁴)₂, —S(═O)(═NR¹⁴)R¹³,
   4- to 7-membered heterocycloalkyl and 5- or 6-membered heteroaryl,
      wherein said 4- to 7-membered heterocycloalkyl group
      is connected to the rest of the molecule via a carbon atom of said 4- to 7-membered heterocycloalkyl group,
      and
      wherein said 5- or 6-membered heteroaryl group is optionally substituted, one or two times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl and —N(R⁹)(R¹⁰),
and stereoisomers, tautomers, N-oxides, hydrates, solvates, and salts thereof, and mixtures of same.

In a further embodiment of the first aspect, the present invention covers compounds of formula (I), supra, in which:
R² represents a 5- to 10-membered heteroaryl group,
   which group is connected to the rest of the molecule via a carbon atom of said group,
   and
   which group is optionally substituted, one, two, three or four times, each substituent independently selected from a halogen atom or a group selected from
   C₁-C₄-alkyl, C₃-C₅-cycloalkyl, C₁-C₄-haloalkyl, C₁-C₄-alkoxy, (C₁-C₂-alkoxy)-(C₁-C₄-alkoxy)-, C₁-C₄-haloalkoxy, —SR¹⁴, —S(═O)R¹⁴, —S(═O)₂R¹⁴, —P(═O)(R¹⁴)₂, nitro, cyano, hydroxy, —N(R⁹)(R¹⁰), —C(═O)N(R⁹)(R¹⁰), —C(═O)₂R¹⁵, —N(R¹²)S(═O)₂R¹⁴, —N═S(═O)(R¹⁴)₂, —S(═O)(═NR¹⁴)R¹³,
   4- to 7-membered heterocycloalkyl and 5- or 6-membered heteroaryl,
      wherein said 4- to 7-membered heterocycloalkyl group
      is connected to the rest of the molecule via a carbon atom of said 4- to 7-membered heterocycloalkyl group,
      and
      wherein said 5- or 6-membered heteroaryl group is optionally substituted, one or two times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl and —N(R⁹)(R¹⁰)
and stereoisomers, tautomers, N-oxides, hydrates, solvates, and salts thereof, and mixtures of same.
In a further embodiment of the first aspect, the present invention covers compounds of formula (I), supra, in which:
R² represents a group selected from phenyl, naphthyl and 5- to 10-membered heteroaryl,
   which 5- to 10-membered heteroaryl group is connected to the rest of the molecule via a carbon atom of said 5- to 10-membered heteroaryl group,
   and
   which phenyl, naphthyl and 5- to 10-membered heteroaryl group is optionally substituted, one, two, three or four times, each substituent independently selected from a halogen atom or a group selected from
   C₁-C₄-alkyl, C₃-C₅-cycloalkyl, C₁-C₄-haloalkyl, C₁-C₄-alkoxy, (C₁-C₂-alkoxy)-(C₁-C₄-alkoxy)-, C₁-C₄-haloalkoxy, —SR¹⁴, —S(═O)R¹⁴, —S(═O)₂R¹⁴, nitro, cyano, hydroxy, —N(R⁹)(R¹⁰), —C(═O)N(R⁹)(R¹⁰), —C(═O)₂R¹⁵, —N═S(═O)(R¹⁴)₂ and 5- or 6-membered heteroaryl,
      wherein said 5- or 6-membered heteroaryl group is optionally substituted, one or two times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl and —N(R⁹)(R¹⁰)
and stereoisomers, tautomers, N-oxides, hydrates, solvates, and salts thereof, and mixtures of same.

In a further embodiment of the first aspect, the present invention covers compounds of formula (I), supra, in which:
R² represents a phenyl group,
   which group is optionally substituted, one, two, three or four times, each substituent independently selected from a halogen atom or a group selected from
   C₁-C₄-alkyl, C₃-C₅-cycloalkyl, C₁-C₄-haloalkyl, C₁-C₄-alkoxy, (C₁-C₂-alkoxy)-(C₁-C₄-alkoxy)-, C₁-C₄-haloalkoxy, —SR¹⁴, —S(═O)R¹⁴, —S(═O)₂R¹⁴, nitro, cyano, hydroxy, —N(R⁹)(R¹⁰), —C(═O)N(R⁹)(R¹⁰), —C(═O)₂R¹⁵, —N═S(═O)(R¹⁴)₂ and 5- or 6-membered heteroaryl,
      wherein said 5- or 6-membered heteroaryl group is optionally substituted, one or two times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl and —N(R⁹)(R¹⁰),
and stereoisomers, tautomers, N-oxides, hydrates, solvates, and salts thereof, and mixtures of
In a further embodiment of the first aspect, the present invention covers compounds of formula (I), supra, in which:
R² represents a 5- to 10-membered heteroaryl group,
   which group is connected to the rest of the molecule via a carbon atom of said group,
   and
   which group is optionally substituted, one, two, three or four times, each substituent independently selected from a halogen atom or a group selected from
   C₁-C₄-alkyl, C₃-C₅-cycloalkyl, C₁-C₄-haloalkyl, C₁-C₄-alkoxy, (C₁-C₂-alkoxy)-(C₁-C₄-alkoxy)-, C₁-C₄-haloalkoxy, —SR¹⁴, —S(═O)R¹⁴, —S(═O)₂R¹⁴, nitro, cyano, hydroxy, —N(R⁹)(R¹⁰), —C(═O)N(R⁹)(R¹⁰), —C(═O)₂R¹⁵, —N═S(═O)(R¹⁴)₂ and 5- or 6-membered heteroaryl,
      wherein said 5- or 6-membered heteroaryl group is optionally substituted, one or two times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl and —N(R⁹)(R¹⁰)
and stereoisomers, tautomers, N-oxides, hydrates, solvates, and salts thereof, and mixtures of same.

In a further embodiment of the first aspect, the present invention covers compounds of formula (I), supra, in which:
R² represents a group selected from phenyl, naphthyl and 5- to 10-membered heteroaryl,
   which 5- to 10-membered heteroaryl group is selected from pyridinyl, pyrimidinyl, 1,2-benzoxazolyl, 1,3-benzoxazolyl and 1,3-benzothiazolyl,
   and
   which phenyl, naphthyl and 5- to 10-membered heteroaryl group is optionally substituted, one, two, three or four times, each substituent independently selected from a halogen atom or a group selected from
   C₁-C₄-alkyl, C₃-C₅-cycloalkyl, C₁-C₄-haloalkyl, C₁-C₄-alkoxy, (C₁-C₂-alkoxy)-(C₁-C₄-alkoxy)-, C₁-C₄-haloalkoxy, —SR¹⁴, —S(═O)R¹⁴, —S(═O)₂R¹⁴, nitro, cyano, hydroxy, —N(R⁹)(R¹⁰), —C(═O)N(R⁹)(R¹⁰), —C(═O)₂R¹⁵, —N═S(═O)(R¹⁴)₂ and 5- or 6-membered heteroaryl,
      wherein said 5- or 6-membered heteroaryl group is optionally substituted, one or two times, each substituent independently selected from a halogen atom or a C₁-C₂-alkyl group,
and stereoisomers, tautomers, N-oxides, hydrates, solvates, and salts thereof, and mixtures of same.

In a further embodiment of the first aspect, the present invention covers compounds of formula (I), supra, in which:
R² represents a phenyl group,
   which group is optionally substituted, one, two, three or four times, each substituent independently selected from a halogen atom or a group selected from
   C₁-C₄-alkyl, C₃-C₅-cycloalkyl, C₁-C₄-haloalkyl, C₁-C₄-alkoxy, (C₁-C₂-alkoxy)-(C₁-C₄-alkoxy)-, C₁-C₄-haloalkoxy, —SR¹⁴, —S(═O)R¹⁴, —S(═O)₂R¹⁴, nitro, cyano, hydroxy, —N(R⁹)(R¹⁰), —C(═O)N(R⁹)(R¹⁰), —C(═O)₂R¹⁵, —N═S(═O)(R¹⁴)₂ and 5- or 6-membered heteroaryl,
      wherein said 5- or 6-membered heteroaryl group is optionally substituted, one or two times, each substituent independently selected from a halogen atom or a C₁-C₂-alkyl group,
and stereoisomers, tautomers, N-oxides, hydrates, solvates, and salts thereof, and mixtures of same.

In a further embodiment of the first aspect, the present invention covers compounds of formula (I), supra, in which:
R² represents a 5- to 10-membered heteroaryl group,
   which 5- to 10-membered heteroaryl group is selected from pyridinyl, pyrimidinyl, 1,2-benzoxazolyl, 1,3-benzoxazolyl and 1,3-benzothiazolyl,
   and
   which group is optionally substituted, one, two, three or four times, each substituent independently selected from a halogen atom or a group selected from
   C₁-C₄-alkyl, C₃-C₅-cycloalkyl, C₁-C₄-haloalkyl, C₁-C₄-alkoxy, (C₁-C₂-alkoxy)-(C₁-C₄-alkoxy)-, C₁-C₄-haloalkoxy, —SR¹⁴, —S(═O)R¹⁴, —S(═O)₂R¹⁴, nitro, cyano, hydroxy, —N(R⁹)(R¹⁰), —C(═O)N(R⁹)(R¹⁰), —C(═O)₂R¹⁵, —N═S(═O)(R¹⁴)₂ and 5- or 6-membered heteroaryl,
      wherein said 5- or 6-membered heteroaryl group is optionally substituted, one or two times, each substituent independently selected from a halogen atom or a C₁-C₂-alkyl group,
and stereoisomers, tautomers, N-oxides, hydrates, solvates, and salts thereof, and mixtures of same.
In a further embodiment of the first aspect, the present invention covers compounds of formula (I), supra, in which:
R² represents a group selected from phenyl, pyridin-2-yl, pyridin-3-yl, pyrimidin-5-yl, 1,2-benzoxazol-6-yl, 1,3-benzoxazol-4-yl and 1,3-benzothiazol-2-yl,
   which group is optionally substituted, one or two times, each substituent independently selected from a fluorine, chlorine, or bromine atom or a group selected from
   methyl, cyclopropyl, difluoromethyl, trifluoromethyl, methoxy, ethoxy, isopropoxy, 2-methoxyethoxy, difluoromethoxy, trifluormethoxy, methylsulfanyl, methylsulfinyl, methylsulfonyl, nitro, cyano, amino, dimethylamino, azetidin-1-yl, 2-oxopyrrolidin-1-yl, 3-methyl-2-oxo-2,3-dihydro-1H-imidazol-1-yl, 2-oxopiperidin-1-yl, 4-methyl-2-oxopiperazin-1-yl, morpholino-4-yl, 3-methyl-2-oxoimidazolidin-1-yl, 3-methyl-2-oxo-1,3-diazinan-1-yl, carbamoyl, dimethylcarbamoyl, ethoxycarbonyl, [dimethyl(oxido)-Δ⁶-sulfanylidene]amino, 1H-imidazol-1-yl, 1-methyl-1H-pyrazol-4-yl and 1H-1,2,4-triazol-1-yl,
and stereoisomers, tautomers, N-oxides, hydrates, solvates, and salts thereof, and mixtures of same.

In a further embodiment of the first aspect, the present invention covers compounds of formula (I), supra, in which:
R² represents a phenyl group,
   which group is optionally substituted, one or two times, each substituent independently selected from a fluorine, chlorine, or bromine atom or a group selected from
   methyl, cyclopropyl, difluoromethyl, trifluoromethyl, methoxy, ethoxy, isopropoxy, 2-methoxyethoxy, difluoromethoxy, trifluormethoxy, methylsulfanyl, methylsulfinyl, methylsulfonyl, nitro, cyano, amino, dimethylamino, azetidin-1-yl, 2-oxopyrrolidin-1-yl, 3-methyl-2-oxo-2,3-dihydro-1H-imidazol-1-yl, 2-oxopiperidin-1-yl, 4-methyl-2-oxopiperazin-1-yl, morpholino-4-yl, 3-methyl-2-oxoimidazolidin-1-yl, 3-methyl-2-oxo-1,3-diazinan-1-yl, carbamoyl, dimethylcarbamoyl, ethoxycarbonyl, [dimethyl(oxido)-Δ⁶-sulfanylidene]amino, 1H-imidazol-1-yl, 1-methyl-1H-pyrazol-4-yl and 1H-1,2,4-triazol-1-yl,
and stereoisomers, tautomers, N-oxides, hydrates, solvates, and salts thereof, and mixtures of same.
In a further embodiment of the first aspect, the present invention covers compounds of formula (I), supra, in which:
R² represents a group selected from pyridin-2-yl, pyridin-3-yl, pyrimidin-5-yl, 1,2-benzoxazol-6-yl, 1,3-benzoxazol-4-yl and 1,3-benzothiazol-2-yl,
   which group is optionally substituted, one or two times, each substituent independently selected from a fluorine, chlorine, or bromine atom or a group selected from
   methyl, cyclopropyl, difluoromethyl, trifluoromethyl, methoxy, ethoxy, isopropoxy, 2-methoxyethoxy, difluoromethoxy, trifluormethoxy, methylsulfanyl, methylsulfinyl, methylsulfonyl, nitro, cyano, amino, dimethylamino, azetidin-1-yl, 2-oxopyrrolidin-1-yl, 3-methyl-2-oxo-2,3-dihydro-1H-imidazol-1-yl, 2-oxopiperidin-1-yl, 4-methyl-2-oxopiperazin-1-yl, morpholino-4-yl, 3-methyl-2-oxoimidazolidin-1-yl, 3-methyl-2-oxo-1,3-diazinan-1-yl, carbamoyl, dimethylcarbamoyl, ethoxycarbonyl, [dimethyl(oxido)-Δ⁶-sulfanylidene]amino, 1H-imidazol-1-yl, 1-methyl-1H-pyrazol-4-yl and 1H-1,2,4-triazol-1-yl,
and stereoisomers, tautomers, N-oxides, hydrates, solvates, and salts thereof, and mixtures of same.

In a further embodiment of the first aspect, the present invention covers compounds of formula (I), supra, in which:
R³ represents a hydrogen atom or a halogen atom or a group selected from
   C₁-C₆-alkyl, C₂-C₆-alkenyl, C₂-C₆-alkynyl, C₃-C₆-cycloalkyl, C₄-C₆-cycloalkenyl, C₁-C₆-hydroxyalkyl, C₁-C₆-haloalkyl, (C₁-C₂-alkoxy)-(C₁-C₆-alkyl)-, C₁-C₆-alkoxy, (C₁-C₂-alkoxy)-(C₁-C₆-alkoxy)-, C₁-C₄-haloalkoxy, C₃-C₆-cycloalkyloxy, phenoxy, —SR¹⁴, —S(═O)R¹⁴, —S(═O)₂R¹⁴, cyano, hydroxy, —N(R⁹)(R¹⁰), —C(═O)N(R⁹)(R¹⁰), —C(═O)R¹¹, —N(R¹²)C(═O)R¹³, —N(R¹²)S(═O)₂R¹⁴, —N═S(═NH)(R¹⁴)₂, —N═S(═O)(R¹⁴)₂, —P(═O)(R¹⁴)₂,
   4- to 7-membered heterocycloalkyl, 5- to 7-membered heterocycloalkenyl,
   (4- to 7-membered heterocycloalkyl)oxy, phenyl and 5- or 6-membered heteroaryl,
      wherein said 4- to 7-membered heterocycloalkyl group and
      5- to 7-membered heterocycloalkenyl group is connected to the rest of the molecule via a carbon atom of said 4- to 7-membered heterocycloalkyl group and 5- to 7-membered heterocycloalkenyl group,
      and
      wherein said 4- to 7-membered heterocycloalkyl group,
      5- to 7-membered heterocycloalkenyl group and
      (4- to 7-membered heterocycloalkyl)oxy group is optionally substituted, one, two or three times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl, —N(R⁹)(R¹⁰) and oxo,
      and
      wherein said C₁-C₆-alkyl, C₂-C₆-alkenyl, C₂-C₆-alkynyl and C₁-C₆-alkoxy group is optionally substituted with a group selected from
      C₃-C₄-cycloalkyl, phenyl and 4- to 7-membered heterocycloalkyl,
         wherein said 4- to 7-membered heterocycloalkyl group, is connected to the rest of the molecule via a carbon atom of said
         4- to 7-membered heterocycloalkyl group,
         and
         wherein said 4- to 7-membered heterocycloalkyl group is optionally substituted, one, two or three times, each substituent independently selected from a halogen atom or a group selected from
         C₁-C₂-alkyl, C₁-C₂-haloalkyl, cyano, hydroxy, C₁-C₂-alkoxy, C₃-C₄-cycloalkyl, —N(R⁹)(R¹⁰) and oxo,
         and
         which phenyl group is optionally substituted, one or two times, each substituent independently selected from a halogen atom or a group selected from
         C₁-C₂-alkyl, C₁-C₂-haloalkyl, cyano, hydroxy, C₁-C₂-alkoxy, C₃-C₄-cycloalkyl and —N(R⁹)(R¹⁰)
         and
         which C₃-C₄-cycloalkyl group is optionally substituted, one or two times, each substituent independently selected from a halogen atom or a group selected from
         cyano and hydroxy,
      and
      wherein said C₃-C₆-cycloalkyl and C₄-C₆-cycloalkenyl group is optionally substituted, one or two times, each substituent independently selected from a halogen atom or a C₁-C₄-alkyl group,
      and
      wherein said phenyl, phenoxy and 5- or 6-membered heteroaryl group is optionally substituted, one or two times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl and —N(R⁹)(R¹⁰),
and stereoisomers, tautomers, N-oxides, hydrates, solvates, and salts thereof, and mixtures of same.
In a further embodiment of the first aspect, the present invention covers compounds of formula (I), supra, in which:
R³ represents a hydrogen atom or a halogen atom or a group selected from
   C₁-C₄-alkyl, C₂-C₄-alkenyl, C₂-C₄-alkynyl, C₃-C₅-cycloalkyl, C₄-C₅-cycloalkenyl, C₁-C₄-hydroxyalkyl, C₁-C₄-haloalkyl, (C₁-C₂-alkoxy)-(C₁-C₄-alkyl)-, C₁-C₄-alkoxy, (C₁-C₂-alkoxy)-(C₁-C₄-alkoxy)-, C₁-C₄-haloalkoxy, C₃-C₅-cycloalkyloxy, phenoxy, —SR¹⁴, —S(═O)R¹⁴, —S(═O)₂R¹⁴, cyano, hydroxy, —N(R⁹)(R¹⁰), —C(═O)N(R⁹)(R¹⁰), —C(═O)R¹¹, —N(R¹²)C(═O)R¹³, —N(R¹²)S(═O)₂R¹⁴, —N═S(═NH)(R¹⁴)₂, —N═S(═O)(R¹⁴)₂, —P(═O)(R¹⁴)₂,
   4- to 7-membered heterocycloalkyl, 5- to 7-membered heterocycloalkenyl,
   (4- to 7-membered heterocycloalkyl)oxy, phenyl and 5- or 6-membered heteroaryl,
      wherein said 4- to 7-membered heterocycloalkyl group and
      5- to 7-membered heterocycloalkenyl group is connected to the rest of the molecule via a carbon atom of said
      4- to 7-membered heterocycloalkyl group and
      5- to 7-membered heterocycloalkenyl group,
      and
      wherein said 4- to 7-membered heterocycloalkyl group,
      5- to 7-membered heterocycloalkenyl group and
      (4- to 7-membered heterocycloalkyl)oxy group is optionally substituted, one, two or three times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl, —N(R⁹)(R¹⁰) and oxo,
      and
      wherein said C₁-C₄-alkyl, C₂-C₄-alkenyl, C₂-C₄-alkynyl and C₁-C₄-alkoxy group is optionally substituted with a group selected from
      C₃-C₄-cycloalkyl, phenyl and 4- to 7-membered heterocycloalkyl,
         wherein said 4- to 7-membered heterocycloalkyl group is connected to the rest of the molecule via a carbon atom of said 4- to 7-membered heterocycloalkyl group,
         and
         wherein said 4- to 7-membered heterocycloalkyl group is optionally substituted, one, two or three times, each substituent independently selected from a halogen atom or a group selected from
         C₁-C₂-alkyl, C₁-C₂-haloalkyl, cyano, hydroxy, C₁-C₂-alkoxy, C₃-C₄-cycloalkyl, —N(R⁹)(R¹⁰) and oxo,
         and
         which phenyl group is optionally substituted, one or two times, each substituent independently selected from a halogen atom or a group selected from
         C₁-C₂-alkyl, C₁-C₂-haloalkyl, cyano, hydroxy, C₁-C₂-alkoxy, C₃-C₄-cycloalkyl and —N(R⁹)(R¹⁰),
         and
         which C₃-C₄-cycloalkyl group is optionally substituted, one or two times, each substituent independently selected from a halogen atom or a group selected from
         cyano and hydroxy,
      and
      wherein said C₃-C₅-cycloalkyl and C₄-C₅-cycloalkenyl group is optionally substituted, one or two times, each substituent independently selected from a halogen atom or a C₁-C₄-alkyl group,
      and
      wherein said phenyl, phenoxy and 5- or 6-membered heteroaryl group is optionally substituted, one or two times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl and —N(R⁹)(R¹⁰),
and stereoisomers, tautomers, N-oxides, hydrates, solvates, and salts thereof, and mixtures of same.

In a further embodiment of the first aspect, the present invention covers compounds of formula (I), supra, in which:
R³ represents a hydrogen atom or a halogen atom or a group selected from
   C₁-C₄-alkyl, C₃-C₅-cycloalkyl, (C₁-C₂-alkoxy)-(C₁-C₄-alkyl)-, C₁-C₄-alkoxy, —S(═O)₂R¹⁴, cyano, hydroxy, —N(R⁹)(R¹⁰) and —P(═O)(R¹⁴)₂,
and stereoisomers, tautomers, N-oxides, hydrates, solvates, and salts thereof, and mixtures of same.

In a further embodiment of the first aspect, the present invention covers compounds of formula (I), supra, in which:
R³ represents a hydrogen atom or a halogen atom or a group selected from
   C₁-C₄-alkyl, C₃-C₅-cycloalkyl, C₁-C₄-alkoxy, cyano and hydroxy,
and stereoisomers, tautomers, N-oxides, hydrates, solvates, and salts thereof, and mixtures of same.
In a further embodiment of the first aspect, the present invention covers compounds of formula (I), supra, in which:
R³ represents a hydrogen atom or a fluorine, chlorine or bromine atom or a group selected from methyl, cyclopropyl, methoxy, cyano and hydroxy,
and stereoisomers, tautomers, N-oxides, hydrates, solvates, and salts thereof, and mixtures of same.

In a further embodiment of the first aspect, the present invention covers compounds of formula (I), supra, in which:
R⁴ represents a hydrogen atom or a halogen atom or a group selected from
   C₁-C₆-alkyl, C₂-C₆-alkenyl, C₂-C₆-alkynyl, C₃-C₆-cycloalkyl, C₄-C₆-cycloalkenyl, C₁-C₆-hydroxyalkyl, C₁-C₆-haloalkyl, (C₁-C₂-alkoxy)-(C₁-C₆-alkyl)-, C₁-C₆-alkoxy, (C₁-C₂-alkoxy)-(C₁-C₆-alkoxy)-, C₁-C₄-haloalkoxy, C₃-C₆-cycloalkyloxy, —S(═O)R¹⁴, —S(═O)₂R¹⁴, cyano, hydroxy, —N(R⁹)(R¹⁰), —N(R¹⁸)(R¹⁹), —C(═O)N(R⁹)(R¹⁰), —C(═O)R¹¹, —N(R¹²)C(═O)R¹³, —N(R¹²)S(═O)₂R¹⁴, —N═S(═NH)(R¹⁴)₂, —N═S(═O)(R¹⁴)₂, —P(═O)(R¹⁴)₂, 4- to 7-membered heterocycloalkyl, 5- to 7-membered heterocycloalkenyl, (4- to 7-membered heterocycloalkyl)oxy, phenyl and 5- or 6-membered heteroaryl,
      wherein said 4- to 7-membered heterocycloalkyl group and 5- to 7-membered heterocycloalkenyl group is connected to the rest of the molecule via a carbon atom of said 4- to 7-membered heterocycloalkyl group and 5- to 7-membered heterocycloalkenyl group,
      and
      wherein said 4- to 7-membered heterocycloalkyl group, 5- to 7-membered heterocycloalkenyl group and (4- to 7-membered heterocycloalkyl)oxy group is optionally substituted, one, two or three times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl, —N(R⁹)(R¹⁰) and oxo,
      and
      wherein said C₁-C₆-alkyl, C₂-C₆-alkenyl, C₂-C₆-alkynyl and C₁-C₆-alkoxy group is optionally substituted with a group selected from
      C₃-C₄-cycloalkyl, phenyl and 4- to 7-membered heterocycloalkyl,
         wherein said 4- to 7-membered heterocycloalkyl group is connected to the rest of the molecule via a carbon atom of said 4- to 7-membered heterocycloalkyl group,
         and
         wherein said 4- to 7-membered heterocycloalkyl group is optionally substituted, one, two or three times, each substituent independently selected from a halogen atom or a group selected from
         C₁-C₂-alkyl, C₁-C₂-haloalkyl, cyano, hydroxy, C₁-C₂-alkoxy, C₃-C₄-cycloalkyl, —N(R⁹)(R¹⁰) and oxo,
         and
         which phenyl group is optionally substituted, one or two times, each substituent independently selected from a halogen atom or a group selected from
         C₁-C₂-alkyl, C₁-C₂-haloalkyl, cyano, hydroxy, C₁-C₂-alkoxy, C₃-C₄-cycloalkyl and —N(R⁹)(R¹⁰),
         and
         which C₃-C₄-cycloalkyl group is optionally substituted, one or two times, each substituent independently selected from a halogen atom or a group selected from
         cyano and hydroxy,
      and
      wherein said C₃-C₆-cycloalkyl and C₄-C₆-cycloalkenyl group is optionally substituted, one or two times, each substituent independently selected from a halogen atom or a C₁-C₄-alkyl group,
      and
      wherein said phenyl and 5- or 6-membered heteroaryl group is optionally substituted, one or two times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl and —N(R⁹)(R¹⁰),
and stereoisomers, tautomers, N-oxides, hydrates, solvates, and salts thereof, and mixtures of same.

In a further embodiment of the first aspect, the present invention covers compounds of formula (I), supra, in which:
R⁴ represents a hydrogen atom or a halogen atom or a group selected from
   C₁-C₄-alkyl, C₂-C₄-alkenyl, C₂-C₄-alkynyl, C₃-C₅-cycloalkyl, C₄-C₅-cycloalkenyl, C₁-C₄-hydroxyalkyl, C₁-C₄-haloalkyl, (C₁-C₂-alkoxy)-(C₁-C₄-alkyl)-, C₁-C₄-alkoxy, (C₁-C₂-alkoxy)-(C₁-C₄-alkoxy)-, C₁-C₄-haloalkoxy, C₃-C₅-cycloalkyloxy, —S(═O)R¹⁴, —S(═O)₂R¹⁴, cyano, hydroxy, —N(R⁹)(R¹⁰), —N(R¹⁸)(R¹⁹), —C(═O)N(R⁹)(R¹⁰), —C(═O)R¹¹, —N(R¹²)C(═O)R¹³, —N(R¹²)S(═O)₂R¹⁴, —N═S(═NH)(R¹⁴)₂, —N═S(═O)(R¹⁴)₂, —P(═O)(R¹⁴)₂, 4- to 7-membered heterocycloalkyl, 5- to 7-membered heterocycloalkenyl, (4- to 7-membered heterocycloalkyl)oxy, phenyl and 5- or 6-membered heteroaryl,
      wherein said 4- to 7-membered heterocycloalkyl group and 5- to 7-membered heterocycloalkenyl group is connected to the rest of the molecule via a carbon atom of said 4- to 7-membered heterocycloalkyl group and 5- to 7-membered heterocycloalkenyl group,
      and
      wherein said 4- to 7-membered heterocycloalkyl group, 5- to 7-membered heterocycloalkenyl group and (4- to 7-membered heterocycloalkyl)oxy group is optionally substituted, one, two or three times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl, —N(R⁹)(R¹⁰) and oxo,
      and
      wherein said C₁-C₄-alkyl, C₂-C₄-alkenyl, C₂-C₄-alkynyl and C₁-C₄-alkoxy group is optionally substituted with a group selected from
      C₃-C₄-cycloalkyl, phenyl and 4- to 7-membered heterocycloalkyl,
         wherein said 4- to 7-membered heterocycloalkyl group is connected to the rest of the molecule via a carbon atom of said 4- to 7-membered heterocycloalkyl group,
         and
         wherein said 4- to 7-membered heterocycloalkyl group is optionally substituted, one, two or three times, each substituent independently selected from a halogen atom or a group selected from
         C₁-C₂-alkyl, C₁-C₂-haloalkyl, cyano, hydroxy, C₁-C₂-alkoxy, C₃-C₄-cycloalkyl, —N(R⁹)(R¹⁰) and oxo,
         and
         which phenyl group is optionally substituted, one or two times, each substituent independently selected from a halogen atom or a group selected from
         C₁-C₂-alkyl, C₁-C₂-haloalkyl, cyano, hydroxy, C₁-C₂-alkoxy, C₃-C₄-cycloalkyl and —N(R⁹)(R¹⁰),
         and
         which C₃-C₄-cycloalkyl group is optionally substituted, one or two times, each substituent independently selected from a halogen atom or a group selected from
         cyano and hydroxy,
      and
      wherein said C₃-C₅-cycloalkyl and C₄-C₅-cycloalkenyl group is optionally substituted, one or two times, each substituent independently selected from a halogen atom or a C₁-C₄-alkyl group,
      and
      wherein said phenyl and 5- or 6-membered heteroaryl group is optionally substituted, one or two times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl and —N(R⁹)(R¹⁰),
and stereoisomers, tautomers, N-oxides, hydrates, solvates, and salts thereof, and mixtures of same.

In a further embodiment of the first aspect, the present invention covers compounds of formula (I), supra, in which:
R⁴ represents a hydrogen atom or a halogen atom or a group selected from
   C₁-C₄-alkyl, C₃-C₅-cycloalkyl, (C₁-C₂-alkoxy)-(C₁-C₄-alkyl)-, C₁-C₄-alkoxy, (C₁-C₂-alkoxy)-(C₁-C₄-alkoxy)-, C₃-C₅-cycloalkyloxy, hydroxy, —N(R⁹)(R¹⁰), —N(R¹⁸)(R¹⁹), —P(═O)(R¹⁴)₂, 4- to 7-membered heterocycloalkyl and (4- to 7-membered heterocycloalkyl)oxy,
      wherein said 4- to 7-membered heterocycloalkyl group is connected to the rest of the molecule via a carbon atom of said 4- to 7-membered heterocycloalkyl group,
      and
      wherein said 4- to 7-membered heterocycloalkyl group and (4- to 7-membered heterocycloalkyl)oxy group is optionally substituted, one, two or three times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl, —N(R⁹)(R¹⁰) and oxo,
and stereoisomers, tautomers, N-oxides, hydrates, solvates, and salts thereof, and mixtures of same.

In a further embodiment of the first aspect, the present invention covers compounds of formula (I), supra, in which:
R⁴ represents a hydrogen atom or a halogen atom or a group selected from
   C₁-C₄-alkyl, C₃-C₅-cycloalkyl, (C₁-C₂-alkoxy)-(C₁-C₄-alkyl)-, C₁-C₄-alkoxy, (C₁-C₂-alkoxy)-(C₁-C₄-alkoxy)-, C₃-C₅-cycloalkyloxy, hydroxy, —N(R⁹)(R¹⁰), —N(R¹⁸)(R¹⁹), —P(═O)(R¹⁴)₂, 4- to 7-membered heterocycloalkyl and (4- to 7-membered heterocycloalkyl)oxy,
      wherein said 4- to 7-membered heterocycloalkyl group is connected to the rest of the molecule via a carbon atom of said 4- to 7-membered heterocycloalkyl group,
and stereoisomers, tautomers, N-oxides, hydrates, solvates, and salts thereof, and mixtures of same.

In a further embodiment of the first aspect, the present invention covers compounds of formula (I), supra, in which:
R⁴ represents a hydrogen atom or a halogen atom or a group selected from
   C₁-C₄-alkyl, C₃-C₅-cycloalkyl, (C₁-C₂-alkoxy)-(C₁-C₄-alkyl)-, C₁-C₄-alkoxy, (C₁-C₂-alkoxy)-(C₁-C₄-alkoxy)-, C₃-C₅-cycloalkyloxy, hydroxy, —N(R⁹)(R¹⁰), —N(R¹⁸)(R¹⁹), —P(═O)(R¹⁴)₂, 4- to 7-membered heterocycloalkyl and (4- to 7-membered heterocycloalkyl)oxy,
      wherein said 4- to 7-membered heterocycloalkyl group is connected to the rest of the molecule via a carbon atom of said 4- to 7-membered heterocycloalkyl group,
and stereoisomers, tautomers, N-oxides, hydrates, solvates, and salts thereof, and mixtures of same.

In a further embodiment of the first aspect, the present invention covers compounds of formula (I), supra, in which:
R⁴ represents a hydrogen atom or a fluorine, chlorine or bromine atom or a group selected from
   methyl, cyclopropyl, 2-methoxyethyl, methoxy, propoxy, 2-methoxyethoxy, cyclopropyloxy, hydroxy, 2-oxopyrrolidin-1-yl, 4-methylpiperazin-1-yl, methyl(tetrahydrofuran-3-yl)amino, dimethylphosphoryl, oxetan-3-yl, (oxetan-3-yl)oxy, tetrahydrofuranyl-3-oxy, (tetrahydro-2H-pyran-3-yl)oxy and (tetrahydro-2H-pyran-4-yl)oxy,
and stereoisomers, tautomers, N-oxides, hydrates, solvates, and salts thereof, and mixtures of same.

In a further embodiment of the first aspect, the present invention covers compounds of formula (I), supra, in which:
R⁵ represents a hydrogen atom or a halogen atom or a group selected from
   C₁-C₆-alkyl, C₂-C₆-alkenyl, C₂-C₆-alkynyl, C₃-C₆-cycloalkyl, C₄-C₆-cycloalkenyl, C₁-C₆-hydroxyalkyl, C₁-C₆-haloalkyl, (C₁-C₂-alkoxy)-(C₁-C₆-alkyl)-, C₁-C₆-alkoxy, (C₁-C₂-alkoxy)-(C₁-C₆-alkoxy), —C₁-C₄-haloalkoxy, C₃-C₆-cycloalkyloxy, phenoxy, —SR¹⁴, —S(═O)R¹⁴, S(═O)₂R¹⁴, cyano, hydroxy, —N(R⁹)(R¹⁰), —C(═O)N(R⁹)(R¹⁰), —C(═O)R¹¹, —N(R¹²)C(═O)R¹³, —N(R¹²)S(═O)₂R¹⁴, —N═S(═NH)(R¹⁴)₂, —N═S(═O)(R¹⁴)₂, —P(═O)(R¹⁴)₂, 4- to 7-membered heterocycloalkyl, 5- to 7-membered heterocycloalkenyl, (4- to 7-membered heterocycloalkyl)oxy, phenyl and 5- or 6-membered heteroaryl,
      wherein said 4- to 7-membered heterocycloalkyl group and 5- to 7-membered heterocycloalkenyl group is connected to the rest of the molecule via a carbon atom of said 4- to 7-membered heterocycloalkyl group and 5- to 7-membered heterocycloalkenyl group,
      and
      wherein said 4- to 7-membered heterocycloalkyl group, 5- to 7-membered heterocycloalkenyl group and (4- to 7-membered heterocycloalkyl)oxy group is optionally substituted, one, two or three times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl, —N(R⁹)(R¹⁰) and oxo,
      and
      wherein said C₁-C₆-alkyl, C₂-C₆-alkenyl, C₂-C₆-alkynyl and C₁-C₆-alkoxy group is optionally substituted with a group selected from
      C₃-C₄-cycloalkyl, phenyl and 4- to 7-membered heterocycloalkyl,
         wherein said 4- to 7-membered heterocycloalkyl group is connected to the rest of the molecule via a carbon atom of said 4- to 7-membered heterocycloalkyl group,
         and
         wherein said 4- to 7-membered heterocycloalkyl group is optionally substituted, one, two or three times, each substituent independently selected from a halogen atom or a group selected from
         C₁-C₂-alkyl, C₁-C₂-haloalkyl, cyano, hydroxy, C₁-C₂-alkoxy, C₃-C₄-cycloalkyl, —N(R⁹)(R¹⁰) and oxo,
         and
         which phenyl group is optionally substituted, one or two times, each substituent independently selected from a halogen atom or a group selected from
         C₁-C₂-alkyl, C₁-C₂-haloalkyl, cyano, hydroxy, C₁-C₂-alkoxy, C₃-C₄-cycloalkyl and —N(R⁹)(R¹⁰)
         and
         which C₃-C₄-cycloalkyl group is optionally substituted, one or two times, each substituent independently selected from a halogen atom or a group selected from
         cyano and hydroxy,
      and
      wherein said C₃-C₆-cycloalkyl and C₄-C₆-cycloalkenyl group is optionally substituted, one or two times, each substituent independently selected from a halogen atom or a C₁-C₄-alkyl group,
      and
      wherein said phenyl, phenoxy and 5- or 6-membered heteroaryl group is optionally substituted, one or two times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl and —N(R⁹)(R¹⁰),
and stereoisomers, tautomers, N-oxides, hydrates, solvates, and salts thereof, and mixtures of same.

In a further embodiment of the first aspect, the present invention covers compounds of formula (I), supra, in which:
R⁵ represents a hydrogen atom or a halogen atom or a group selected from
   C₁-C₄-alkyl, C₂-C₄-alkenyl, C₂-C₄-alkynyl, C₃-C₅-cycloalkyl, C₄-C₅-cycloalkenyl, C₁-C₄-hydroxyalkyl, C₁-C₄-haloalkyl, (C₁-C₂-alkoxy)-(C₁-C₄-alkyl)-, C₁-C₄-alkoxy, (C₁-C₂-alkoxy)-(C₁-C₄-alkoxy), —C₁-C₄-haloalkoxy, C₃-C₅-cycloalkyloxy, phenoxy, —SR¹⁴, —S(═O)R¹⁴, S(═O)₂R¹⁴, cyano, hydroxy, —N(R⁹)(R¹⁰), —C(═O)N(R⁹)(R¹⁰), —C(═O)R¹, —N(R¹²)C(═O)R¹³, —N(R¹²)S(═O)₂R¹⁴, —N═S(═NH)(R¹⁴)₂, —N═S(═O)(R¹⁴)₂, —P(═O)(R¹⁴)₂, 4- to 7-membered heterocycloalkyl, 5- to 7-membered heterocycloalkenyl, (4- to 7-membered heterocycloalkyl)oxy, phenyl and 5- or 6-membered heteroaryl,
      wherein said 4- to 7-membered heterocycloalkyl group and
      5- to 7-membered heterocycloalkenyl group is connected to the rest of the molecule via a carbon atom of said
      4- to 7-membered heterocycloalkyl group and
      5- to 7-membered heterocycloalkenyl group,
      and
      wherein said 4- to 7-membered heterocycloalkyl group,
      5- to 7-membered heterocycloalkenyl group and
      (4- to 7-membered heterocycloalkyl)oxy group is optionally substituted, one, two or three times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl, —N(R⁹)(R¹⁰) and oxo,
      and
      wherein said C₁-C₄-alkyl, C₂-C₄-alkenyl, C₂-C₄-alkynyl and C₁-C₄-alkoxy group is optionally substituted with a group selected from
      C₃-C₄-cycloalkyl, phenyl and 4- to 7-membered heterocycloalkyl,
         wherein said 4- to 7-membered heterocycloalkyl group is connected to the rest of the molecule via a carbon atom of said 4- to 7-membered heterocycloalkyl group,
         and
         wherein said 4- to 7-membered heterocycloalkyl group is optionally substituted, one, two or three times, each substituent independently selected from a halogen atom or a group selected from
         C₁-C₂-alkyl, C₁-C₂-haloalkyl, cyano, hydroxy, C₁-C₂-alkoxy, C₃-C₄-cycloalkyl, —N(R⁹)(R¹⁰) and oxo,
         and
         which phenyl group is optionally substituted, one or two times, each substituent independently selected from a halogen atom or a group selected from
         C₁-C₂-alkyl, C₁-C₂-haloalkyl, cyano, hydroxy, C₁-C₂-alkoxy, C₃-C₄-cycloalkyl and —N(R⁹)(R¹⁰)
         and
         which C₃-C₄-cycloalkyl group is optionally substituted, one or two times, each substituent independently selected from a halogen atom or a group selected from
         cyano and hydroxy,
      and
      wherein said C₃-C₅-cycloalkyl and C₄-C₅-cycloalkenyl group is optionally substituted, one or two times, each substituent independently selected from a halogen atom or a C₁-C₄-alkyl group,
      and
      wherein said phenyl, phenoxy and 5- or 6-membered heteroaryl group is optionally substituted, one or two times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl and —N(R⁹)(R¹⁰),
and stereoisomers, tautomers, N-oxides, hydrates, solvates, and salts thereof, and mixtures of same.

In a further embodiment of the first aspect, the present invention covers compounds of formula (I), supra, in which:
R⁵ represents a hydrogen atom or a halogen atom or a group selected from
   C₁-C₄-alkyl, C₃-C₅-cycloalkyl, C₁-C₄-alkoxy, S(═O)₂R¹⁴, cyano, —N(R⁹)(R¹⁰), —N═S(═O)(R¹⁴)₂ and —P(═O)(R¹⁴)₂,
and stereoisomers, tautomers, N-oxides, hydrates, solvates, and salts thereof, and mixtures of same.

In a further embodiment of the first aspect, the present invention covers compounds of formula (I), supra, in which:
R⁵ represents a hydrogen atom or a halogen atom or a group selected from S(═O)₂R¹⁴, cyano, —N(R⁹)(R¹⁰), —N═S(═O)(R¹⁴)₂ and —P(═O)(R¹⁴)₂,
and stereoisomers, tautomers, N-oxides, hydrates, solvates, and salts thereof, and mixtures of same.

In a further embodiment of the first aspect, the present invention covers compounds of formula (I), supra, in which:
R⁵ represents a hydrogen atom or a fluorine, chlorine or bromine atom or a group selected from methanesulfonyl, cyano, 2-oxopyrrolidin-1-yl, [dimethyl(oxido)-Δ⁶-sulfanylidene]amino and dimethylphosphoryl,
and stereoisomers, tautomers, N-oxides, hydrates, solvates, and salts thereof, and mixtures of same.

In a further embodiment of the first aspect, the present invention covers compounds of formula (I), supra, in which:
R⁶ represents a hydrogen atom, or a fluorine atom or a group selected from
   C₁-C₄-alkyl, C₁-C₄-hydroxyalkyl, C₁-C₄-alkoxy, hydroxy and oxo,
and stereoisomers, tautomers, N-oxides, hydrates, solvates, and salts thereof, and mixtures of same.

In a further embodiment of the first aspect, the present invention covers compounds of formula (I), supra, in which:
R⁶ represents a hydrogen atom,
and stereoisomers, tautomers, N-oxides, hydrates, solvates, and salts thereof, and mixtures of

In a further embodiment of the first aspect, the present invention covers compounds of formula (I), supra, in which:
R⁷ represents a hydrogen atom or a halogen atom or a group selected from
   C₁-C₄-alkyl, C₁-C₄-alkoxy, hydroxy and cyano,
and stereoisomers, tautomers, N-oxides, hydrates, solvates, and salts thereof, and mixtures of same.

In a further embodiment of the first aspect, the present invention covers compounds of formula (I), supra, in which:
R⁷ represents a hydrogen atom or a C₁-C₄-alkyl group,
and stereoisomers, tautomers, N-oxides, hydrates, solvates, and salts thereof, and mixtures of same.

In a further embodiment of the first aspect, the present invention covers compounds of formula (I), supra, in which:
R⁷ represents a hydrogen atom or a methyl group,
and stereoisomers, tautomers, N-oxides, hydrates, solvates, and salts thereof, and mixtures of same.

In a further embodiment of the first aspect, the present invention covers compounds of formula (I), supra, in which:
R³ represents a group selected from methyl and ethyl,
and stereoisomers, tautomers, N-oxides, hydrates, solvates, and salts thereof, and mixtures of same.

In a further embodiment of the first aspect, the present invention covers compounds of formula (I), supra, in which:
R³ represents a methyl group,
and stereoisomers, tautomers, N-oxides, hydrates, solvates, and salts thereof, and mixtures of same.

In a further embodiment of the first aspect, the present invention covers compounds of formula (I), supra, in which:
R⁹ and R¹⁰ represent, independently from each occurrence, a hydrogen atom or a group selected from
   C₁-C₄-alkyl, (C₁-C₄-alkoxy)-(C₂-C₄-alkyl)-, C₃-C₄-cycloalkyl and C₂-C₄-haloalkyl,
   or
R⁹ and R¹⁰ together with the nitrogen to which they are attached represent a nitrogen containing 4- to 7-membered heterocycloalkyl group or 5- to 7-membered heterocycloalkenyl group,
   wherein said nitrogen containing 4- to 7-membered heterocycloalkyl group and 5- to 7-membered heterocycloalkenyl group are optionally substituted, one, two or three times, each substituent independently selected from a halogen atom or a group selected from
   C₁-C₄-alkyl, C₃-C₄-cycloalkyl, hydroxy and oxo,
   or
   two substituents, which are attached to the same carbon atom of said nitrogen containing 4- to 7-membered heterocycloalkyl group, together with the carbon atom to which they are attached, represent a 4- to 7-membered heterocycloalkyl group,
      wherein said 4- to 7-membered heterocycloalkyl group is optionally substituted, one or two times, each substituent independently selected from a halogen atom or a group selected from
      C₁-C₄-alkyl, C₃-C₄-cycloalkyl, C₁-C₄-haloalkyl, hydroxy and oxo,
and stereoisomers, tautomers, N-oxides, hydrates, solvates, and salts thereof, and mixtures of same.

In a further embodiment of the first aspect, the present invention covers compounds of formula (I), supra, in which:
R⁹ and R¹⁰ represent, independently from each occurrence, a hydrogen atom or a group selected from
   C₁-C₄-alkyl, (C₁-C₄-alkoxy)-(C₂-C₄-alkyl)-, C₃-C₄-cycloalkyl and C₂-C₄-haloalkyl,
and stereoisomers, tautomers, N-oxides, hydrates, solvates, and salts thereof, and mixtures of same.

In a further embodiment of the first aspect, the present invention covers compounds of formula (I), supra, in which:
R⁹ and R¹⁰ together with the nitrogen to which they are attached represent a nitrogen containing 4- to 7-membered heterocycloalkyl group or 5- to 7-membered heterocycloalkenyl group,
   wherein said nitrogen containing 4- to 7-membered heterocycloalkyl group and 5- to 7-membered heterocycloalkenyl group are optionally substituted, one, two or three times, each substituent independently selected from a halogen atom or a group selected from
   C₁-C₄-alkyl, C₃-C₄-cycloalkyl, hydroxy and oxo,
   or
   two substituents, which are attached to the same carbon atom of said nitrogen containing 4- to 7-membered heterocycloalkyl group, together with the carbon atom to which they are attached, represent a 4- to 7-membered heterocycloalkyl group,
      wherein said 4- to 7-membered heterocycloalkyl group is optionally substituted, one or two times, each substituent independently selected from a halogen atom or a group selected from
      C₁-C₄-alkyl, C₃-C₄-cycloalkyl, C₁-C₄-haloalkyl, hydroxy and oxo,
and stereoisomers, tautomers, N-oxides, hydrates, solvates, and salts thereof, and mixtures of same.

In a further embodiment of the first aspect, the present invention covers compounds of formula (I), supra, in which:
R⁹ and R¹⁰ represent, independently from each occurrence, a hydrogen atom or C₁-C₄-alkyl group,
   or
R⁹ and R¹⁰ together with the nitrogen to which they are attached represent a nitrogen containing 4- to 7-membered heterocycloalkyl group or 5- to 7-membered heterocycloalkenyl group,
   wherein said nitrogen containing 4- to 7-membered heterocycloalkyl group and 5- to 7-membered heterocycloalkenyl group are optionally substituted, one, two or three times, each substituent independently selected from a halogen atom or a group selected from
   C₁-C₄-alkyl, C₃-C₄-cycloalkyl, hydroxy and oxo,
   or
   two substituents, which are attached to the same carbon atom of said nitrogen containing 4- to 7-membered heterocycloalkyl group, together with the carbon atom to which they are attached, represent a 4- to 7-membered heterocycloalkyl group,
      wherein said 4- to 7-membered heterocycloalkyl group is optionally substituted, one or two times, each substituent independently selected from a halogen atom or a group selected from
      C₁-C₄-alkyl, C₃-C₄-cycloalkyl, C₁-C₄-haloalkyl, hydroxy and oxo,
and stereoisomers, tautomers, N-oxides, hydrates, solvates, and salts thereof, and mixtures of same.

In a further embodiment of the first aspect, the present invention covers compounds of formula (I), supra, in which:
R⁹ and R¹⁰ represent, independently from each occurrence, a hydrogen atom or C₁-C₄-alkyl group,
and stereoisomers, tautomers, N-oxides, hydrates, solvates, and salts thereof, and mixtures of same.

In a further embodiment of the first aspect, the present invention covers compounds of formula (I), supra, in which:
R⁹ and R¹⁰ represent, independently from each occurrence, a hydrogen atom or C₁-C₄-alkyl group,
   or
R⁹ and R¹⁰ together with the nitrogen to which they are attached represent a nitrogen containing 4- to 7-membered heterocycloalkyl group or 5- to 7-membered heterocycloalkenyl group,
   wherein said nitrogen containing 4- to 7-membered heterocycloalkyl group and 5- to 7-membered heterocycloalkenyl group are optionally substituted, one, two or three times, each substituent independently selected from a halogen atom or a group selected from
   C₁-C₄-alkyl and oxo,
and stereoisomers, tautomers, N-oxides, hydrates, solvates, and salts thereof, and mixtures of same.

In a further embodiment of the first aspect, the present invention covers compounds of formula (I), supra, in which:
R⁹ and R¹⁰ together with the nitrogen to which they are attached represent a nitrogen containing 4- to 7-membered heterocycloalkyl group or 5- to 7-membered heterocycloalkenyl group,
   wherein said nitrogen containing 4- to 7-membered heterocycloalkyl group and 5- to 7-membered heterocycloalkenyl group are optionally substituted, one, two or three times, each substituent independently selected from a halogen atom or a group selected from
   C₁-C₄-alkyl and oxo,
and stereoisomers, tautomers, N-oxides, hydrates, solvates, and salts thereof, and mixtures of same.

In a further embodiment of the first aspect, the present invention covers compounds of formula (I), supra, in which:
R¹¹ represents a hydrogen atom or group selected from
   C₁-C₄-alkyl, C₁-C₄-hydroxyalkyl, C₁-C₄-haloalkyl, phenyl and 5- or 6-membered heteroaryl,
   wherein said phenyl group and 5- or 6-membered heteroaryl group is optionally substituted, one or two times, each substituent independently selected from a halogen atom or a group selected from
   C₁-C₂-alkyl, C₁-C₂-haloalkyl, cyano, hydroxy, C₁-C₂-alkoxy, C₃-C₄-cycloalkyl and —N(R⁹)(R¹⁰),and stereoisomers, tautomers, N-oxides, hydrates, solvates, and salts thereof, and mixtures of same.

In a further embodiment of the first aspect, the present invention covers compounds of formula (I), supra, in which:
R¹¹ represents a hydrogen atom or group selected from C₁-C₄-alkyl, C₁-C₄-hydroxyalkyl, C₁-C₄-haloalkyl, phenyl and 5- or 6-membered heteroaryl,
   wherein said phenyl group and 5- or 6-membered heteroaryl group is optionally substituted, one or two times, each substituent independently selected from a halogen atom or a group selected from
   C₁-C₂-alkyl, C₁-C₂-haloalkyl, cyano, hydroxy, C₁-C₂-alkoxy, C₃-C₄-cycloalkyl and —N(R⁹)(R¹⁰),
and stereoisomers, tautomers, N-oxides, hydrates, solvates, and salts thereof, and mixtures of same.

In a further embodiment of the first aspect, the present invention covers compounds of formula (I), supra, in which:
R¹² represents a hydrogen atom or a C₁-C₄-alkyl group,
and stereoisomers, tautomers, N-oxides, hydrates, solvates, and salts thereof, and mixtures of same.

In a further embodiment of the first aspect, the present invention covers compounds of formula (I), supra, in which:
R¹³ represents a hydrogen atom or a group selected from
   C₁-C₄-alkyl, phenyl and 5- or 6-membered heteroaryl,
      wherein said phenyl group and 5- or 6-membered heteroaryl group is optionally substituted, one or two times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl and —N(R⁹)(R¹⁰),
and stereoisomers, tautomers, N-oxides, hydrates, solvates, and salts thereof, and mixtures of same.

In a further embodiment of the first aspect, the present invention covers compounds of formula (I), supra, in which:
R¹⁴ represents a group selected from C₁-C₄-alkyl, C₁-C₄-haloalkyl, C₃-C₅-cycloalkyl, phenyl and 5- or 6-membered heteroaryl,
   wherein said phenyl group and 5- or 6-membered heteroaryl group is optionally substituted, one or two times, each substituent independently selected from a halogen atom or a group selected from
   C₁-C₂-alkyl, C₁-C₂-haloalkyl, cyano, hydroxy, C₁-C₂-alkoxy, C₃-C₄-cycloalkyl and —N(R⁹)(R¹⁰),
and stereoisomers, tautomers, N-oxides, hydrates, solvates, and salts thereof, and mixtures of same.

In a further embodiment of the first aspect, the present invention covers compounds of formula (I), supra, in which:
R¹⁴ represents a C₁-C₄-alkyl group,
and stereoisomers, tautomers, N-oxides, hydrates, solvates, and salts thereof, and mixtures of same.

In a further embodiment of the first aspect, the present invention covers compounds of formula (I), supra, in which:
R¹⁵ represents a hydrogen atom or a C₁-C₄-alkyl group,
and stereoisomers, tautomers, N-oxides, hydrates, solvates, and salts thereof, and mixtures of same.

In a further embodiment of the first aspect, the present invention covers compounds of formula (I), supra, in which:
R¹⁶ represent, indepently from each other, a hydrogen atom or a halogen atom or a group selected from
   C₁-C₂-alkyl and C₁-C₂-haloalkyl,
and stereoisomers, tautomers, N-oxides, hydrates, solvates, and salts thereof, and mixtures of same.

In a further embodiment of the first aspect, the present invention covers compounds of formula (I), supra, in which:
R¹⁶ represent, indepently from each other, a hydrogen atom or a C₁-C₂-alkyl group,
and stereoisomers, tautomers, N-oxides, hydrates, solvates, and salts thereof, and mixtures of same.

In a further embodiment of the first aspect, the present invention covers compounds of formula (I), supra, in which:
R¹⁶ represent, indepently from each other, a hydrogen atom or a methyl group,
and stereoisomers, tautomers, N-oxides, hydrates, solvates, and salts thereof, and mixtures of same.

In a further embodiment of the first aspect, the present invention covers compounds of formula (I), supra, in which:
R¹⁷ represent, indepently from each other, a hydrogen atom or a halogen atom or a group selected from
   C₁-C₂-alkyl, C₁-C₂-haloalkyl, C₁-C₂-alkoxy, C₁-C₂-haloalkoxy and hydroxy,
and stereoisomers, tautomers, N-oxides, hydrates, solvates, and salts thereof, and mixtures of same.

In a further embodiment of the first aspect, the present invention covers compounds of formula (I), supra, in which:
R¹⁷ represents a hydroxy group,
and stereoisomers, tautomers, N-oxides, hydrates, solvates, and salts thereof, and mixtures of same.

In a further embodiment of the first aspect, the present invention covers compounds of formula (I), supra, in which:
R¹⁸ represents a hydrogen atom or a group selected from
   C₁-C₄-alkyl, C₃-C₄-cycloalkyl and C₂-C₄-haloalkyl,
and stereoisomers, tautomers, N-oxides, hydrates, solvates, and salts thereof, and mixtures of same.

In a further embodiment of the first aspect, the present invention covers compounds of formula (I), supra, in which:
R¹⁸ represents a hydrogen atom or a C₁-C₄-alkyl group,
and stereoisomers, tautomers, N-oxides, hydrates, solvates, and salts thereof, and mixtures of same.

In a further embodiment of the first aspect, the present invention covers compounds of formula (I), supra, in which:
R¹⁹ represents a 4- to 7-membered heterocycloalkyl group,
   wherein said 4- to 7-membered heterocycloalkyl group is optionally substituted, one, two or three times, each substituent independently selected from a halogen atom or a group selected from
   C₁-C₄-alkyl, C₃-C₄-cycloalkyl, C₁-C₄-alkoxy, hydroxy and oxo,
   and
   wherein said 4- to 7-membered heterocycloalkyl group is connected to the rest of the molecule via a carbon atom of the 4- to 7-membered heterocycloalkyl group,
and stereoisomers, tautomers, N-oxides, hydrates, solvates, and salts thereof, and mixtures of same.

In a further embodiment of the first aspect, the present invention covers compounds of formula (I), supra, in which:
R¹⁹ represents a 4- to 7-membered heterocycloalkyl group,
   wherein said 4- to 7-membered heterocycloalkyl group is optionally substituted, one or two times, with a C₁-C₄-alkyl group,
   and
   wherein said 4- to 7-membered heterocycloalkyl group is connected to the rest of the molecule via a carbon atom of the 4- to 7-membered heterocycloalkyl group,
and stereoisomers, tautomers, N-oxides, hydrates, solvates, and salts thereof, and mixtures of same.

In a further embodiment of the first aspect, the present invention covers compounds of formula (I), supra, in which:
R¹⁹ represents a 4- to 7-membered heterocycloalkyl group,
   wherein said 4- to 7-membered heterocycloalkyl group is connected to the rest of the molecule via a carbon atom of the 4- to 7-membered heterocycloalkyl group,
and stereoisomers, tautomers, N-oxides, hydrates, solvates, and salts thereof, and mixtures of same.

In a further embodiment of the first aspect, the present invention covers compounds of formula (I), supra, in which:
o represents an integer selected from 1 or 2,
and stereoisomers, tautomers, N-oxides, hydrates, solvates, and salts thereof, and mixtures of same.

In a further embodiment of the first aspect, the present invention covers compounds of formula (I), supra, in which:
o represents an integer of 1,
and stereoisomers, tautomers, N-oxides, hydrates, solvates, and salts thereof, and mixtures of same.

In a further embodiment of the first aspect, the present invention covers compounds of formula (I), supra, in which:
and
p represents an integer selected from 0 and 1,
and stereoisomers, tautomers, N-oxides, hydrates, solvates, and salts thereof, and mixtures of same.

In a further embodiment of the first aspect, the present invention covers compounds of formula (I), supra, in which:
and
p represents an integer of 0,
and stereoisomers, tautomers, N-oxides, hydrates, solvates, and salts thereof, and mixtures of same.

In a further embodiment of the first aspect, the present invention covers compounds of formula (I), supra, in which:
n represents an integer selected from 1, 2 and 3,
and stereoisomers, tautomers, N-oxides, hydrates, solvates, and salts thereof, and mixtures of same.

In a further embodiment of the first aspect, the present invention covers compounds of formula (I), supra, in which:
n represents an integer selected from 1 and 2,
and stereoisomers, tautomers, N-oxides, hydrates, solvates, and salts thereof, and mixtures of same.

In a further embodiment of the first aspect, the present invention covers compounds of formula (I), supra, in which:
n represents an integer selected from 1 and 3,
and stereoisomers, tautomers, N-oxides, hydrates, solvates, and salts thereof, and mixtures of same.

In a further embodiment of the first aspect, the present invention covers compounds of formula (I), supra, in which:
n represents an integer selected from 2 and 3,
and stereoisomers, tautomers, N-oxides, hydrates, solvates, and salts thereof, and mixtures of same.

In a further embodiment of the first aspect, the present invention covers compounds of formula (I), supra, in which:
n represents an integer of 1,
and stereoisomers, tautomers, N-oxides, hydrates, solvates, and salts thereof, and mixtures of same.

In a further embodiment of the first aspect, the present invention covers compounds of formula (I), supra, in which:
n represents an integer of 2,
and stereoisomers, tautomers, N-oxides, hydrates, solvates, and salts thereof, and mixtures of same.

In a further embodiment of the first aspect, the present invention covers compounds of formula (I), supra, in which:
n represents an integer of 3,
and stereoisomers, tautomers, N-oxides, hydrates, solvates, and salts thereof, and mixtures of same.

In a particular further embodiment of the first aspect, the present invention covers combinations of two or more of the above mentioned embodiments under the heading “further embodiments of the first aspect of the present invention”.

The present invention covers any sub-combination within any embodiment or aspect of the present invention of compounds of general formula (I), supra.

The present invention covers the compounds of general formula (I) which are disclosed in the Example Section of this text, infra.

The compounds of general formula (I) of the present invention can be converted to any salt, preferably pharmaceutically acceptable salts, as described herein, by any method which is known to the person skilled in the art. Similarly, any salt of a compound of general formula (I) of the present invention can be converted into the free compound, by any method which is known to the person skilled in the art.

Compounds of general formula (I) of the present invention demonstrate a valuable pharmacological spectrum of action, which could not have been predicted. Compounds of the present invention have surprisingly been found to effectively inhibit DGKα and it is possible therefore that said compounds be used for the treatment or prophylaxis of diseases, preferably conditions with dysregulated immune responses, particularly cancer or other disorders associated with aberrant DGKα signaling, in humans and animals.

Disorders and conditions particularly suitable for treatment with an DGKα inhibitor of the present invention are liquid and solid tumours, such as cancers of the breast, respiratory tract, brain, reproductive organs, digestive tract, urinary tract, eye, liver, skin, head and neck, thyroid, parathyroid and their distant metastases. Those disorders also include lymphomas, sarcomas, and leukaemias.

Examples of breast cancers include, but are not limited to, triple negative breast cancer, invasive ductal carcinoma, invasive lobular carcinoma, ductal carcinoma in situ, and lobular carcinoma in situ.

Examples of cancers of the respiratory tract include, but are not limited to, small-cell and non-small-cell lung carcinoma, as well as bronchial adenoma and pleuropulmonary blastoma. Examples of brain cancers include, but are not limited to, brain stem and hypophtalmic glioma, cerebellar and cerebral astrocytoma, glioblastoma, medulloblastoma, ependymoma, as well as neuroectodermal and pineal tumour.

Tumours of the male reproductive organs include, but are not limited to, prostate and testicular cancer.

Tumours of the female reproductive organs include, but are not limited to, endometrial, cervical, ovarian, vaginal, and vulvar cancer, as well as sarcoma of the uterus.

Examples of ovarian cancer include, but are not limited to serous tumour, endometrioid tumour, mucinous cystadenocarcinoma, granulosa cell tumour, Sertoli-Leydig cell tumour and arrhenoblastoma.

Examples of cervical cancer include, but are not limited to squamous cell carcinoma, adenocarcinoma, adenosquamous carcinoma, small cell carcinoma, neuroendocrine tumour, glassy cell carcinoma and villoglandular adenocarcinoma.

Tumours of the digestive tract include, but are not limited to, anal, colon, colorectal, esophageal, gallbladder, gastric, pancreatic, rectal, small-intestine, and salivary gland cancers.

Examples of esophageal cancer include, but are not limited to esophageal cell carcinomas and adenocarcinomas, as well as squamous cell carcinomas, leiomyosarcoma, malignant melanoma, rhabdomyosarcoma and lymphoma.

Examples of gastric cancer include, but are not limited to intestinal type and diffuse type gastric adenocarcinoma.

Examples of pancreatic cancer include, but are not limited to ductal adenocarcinoma, adenosquamous carcinomas and pancreatic endocrine tumours.

Tumours of the urinary tract include, but are not limited to, bladder, penile, kidney, renal pelvis, ureter, urethral and human papillary renal cancers.

Examples of kidney cancer include, but are not limited to renal cell carcinoma, urothelial cell carcinoma, juxtaglomerular cell tumour (reninoma), angiomyolipoma, renal oncocytoma, Bellini duct carcinoma, clear-cell sarcoma of the kidney, mesoblastic nephroma and Wilms' tumour.

Examples of bladder cancer include, but are not limited to transitional cell carcinoma, squamous cell carcinoma, adenocarcinoma, sarcoma and small cell carcinoma.

Eye cancers include, but are not limited to, intraocular melanoma and retinoblastoma.

Examples of liver cancers include, but are not limited to, hepatocellular carcinoma (liver cell carcinomas with or without fibrolamellar variant), cholangiocarcinoma (intrahepatic bile duct carcinoma), and mixed hepatocellular cholangiocarcinoma.

Skin cancers include, but are not limited to, squamous cell carcinoma, Kaposi's sarcoma, malignant melanoma, Merkel cell skin cancer, and non-melanoma skin cancer.

Head-and-neck cancers include, but are not limited to, squamous cell cancer of the head and neck, laryngeal, hypopharyngeal, nasopharyngeal, oropharyngeal cancer, salivary gland cancer, lip and oral cavity cancer and squamous cell.

Lymphomas include, but are not limited to, AIDS-related lymphoma, non-Hodgkin's lymphoma, cutaneous T-cell lymphoma, Burkitt lymphoma, Hodgkin's disease, and lymphoma of the central nervous system.

Sarcomas include, but are not limited to, sarcoma of the soft tissue, osteosarcoma, malignant fibrous histiocytoma, lymphosarcoma, and rhabdomyosarcoma.

Leukemias include, but are not limited to, acute myeloid leukemia, acute lymphoblastic leukemia, chronic lymphocytic leukemia, chronic myelogenous leukemia, and hairy cell leukemia.

The term “treating” or “treatment” as stated throughout this document is used conventionally, for example the management or care of a subject for the purpose of combating, alleviating, reducing, relieving, improving the condition of a disease or disorder, such as a carcinoma.

The compounds of the present invention can be used in particular in therapy and prevention, i.e. prophylaxis, of tumour growth and metastases, especially in solid tumours of all indications and stages with or without pre-treatment of the tumour growth.

Generally, the use of chemotherapeutic agents and/or anti-cancer agents in combination with a compound or pharmaceutical composition of the present invention will serve to:
1. yield better efficacy in reducing the growth of a tumour or even eliminate the tumour as compared to administration of either agent alone,
2. provide for the administration of lesser amounts of the administered chemotherapeutic agents,
3. provide for a chemotherapeutic treatment that is well tolerated in the patient with fewer deleterious pharmacological complications than observed with single agent chemotherapies and certain other combined therapies,
4. provide for treating a broader spectrum of different cancer types in mammals, especially humans,
5. provide for a higher response rate among treated patients,
6. provide for a longer survival time among treated patients compared to standard chemotherapy treatments,
7. provide a longer time for tumour progression, and/or
8. yield efficacy and tolerability results at least as good as those of the agents used alone, compared to known instances where other cancer agent combinations produce antagonistic effects.

In addition, the compounds of general formula (I) of the present invention can also be used in combination with radiotherapy and/or surgical intervention.

In a further embodiment of the present invention, the compounds of general formula (I) of the present invention are used in combination with radiation: i.e. radiation treatment sensitizes cancers to anti-tumor immune responses by induction of tumor cell death and subsequent presentation of tumor neoantigens to tumor-reactive Tcells. As DGKα is enhancing the antigen specific activation of T cells, the overall effect results in a much stronger cancer cell attack as compared to irradiation treatment alone.

Thus, the present invention also provides a method of killing a tumor, wherein conventional radiation therapy is employed previous to administering one or more of the compounds of the present invention.

The compounds of the present invention can be administered as the sole pharmaceutical agent or in combination with one or more other pharmaceutically active ingredients where the combination causes no unacceptable adverse effects. The present invention also covers such pharmaceutical combinations. For example, the compounds of the present invention can be combined with:

131I-chTNT, abarelix, abemaciclib, abiraterone, acalabrutinib, aclarubicin, adalimumab, ado-trastuzumab emtansine, afatinib, aflibercept, aldesleukin, alectinib, alemtuzumab, alendronic acid, alitretinoin, alpharadin, altretamine, amifostine, aminoglutethimide, hexyl aminolevulinate, amrubicin, amsacrine, anastrozole, ancestim, anethole dithiolethione, anetumab ravtansine, angiotensin II, antithrombin III, apalutamide, aprepitant, arcitumomab, arglabin, arsenic trioxide, asparaginase, atezolizumab, avelumab, axicabtagene ciloleucel, axitinib, azacitidine, basiliximab, belotecan, bendamustine, besilesomab, belinostat, bevacizumab, bexarotene, bicalutamide, bisantrene, bleomycin, blinatumomab, bortezomib, bosutinib, buserelin, brentuximab vedotin, brigatinib, busulfan, cabazitaxel, cabozantinib, calcitonine, calcium folinate, calcium levofolinate, capecitabine, capromab, carbamazepine carboplatin, carboquone, carfilzomib, carmofur, carmustine, catumaxomab, celecoxib, celmoleukin, cemiplimab, ceritinib, cetuximab, chlorambucil, chlormadinone, chlormethine, cidofovir, cinacalcet, cisplatin, cladribine, clodronic acid, clofarabine, cobimetinib, copanlisib, crisantaspase, crizotinib, cyclophosphamide, cyproterone, cytarabine, dacarbazine, dactinomycin, daratumumab, darbepoetin alfa, dabrafenib, dasatinib, daunorubicin, decitabine, degarelix, denileukin diftitox, denosumab, depreotide, deslorelin, dianhydrogalactitol, dexrazoxane, dibrospidium chloride, dianhydrogalactitol, diclofenac, dinutuximab, docetaxel, dolasetron, doxifluridine, doxorubicin, doxorubicin+estrone, dronabinol, durvalumab, eculizumab, edrecolomab, elliptinium acetate, elotuzumab, eltrombopag, enasidenib, endostatin, enocitabine, enzalutamide, epirubicin, epitiostanol, epoetin alfa, epoetin beta, epoetin zeta, eptaplatin, eribulin, erlotinib, esomeprazole, estradiol, estramustine, ethinylestradiol, etoposide, everolimus, exemestane, fadrozole, fentanyl, filgrastim, fluoxymesterone, floxuridine, fludarabine, fluorouracil, flutamide, folinic acid, formestane, fosaprepitant, fotemustine, fulvestrant, gadobutrol, gadoteridol, gadoteric acid meglumine, gadoversetamide, gadoxetic acid, gallium nitrate, ganirelix, gefitinib, gemcitabine, gemtuzumab, Glucarpidase, glutoxim, GM-CSF, goserelin, granisetron, granulocyte colony stimulating factor, histamine dihydrochloride, histrelin, hydroxycarbamide, I-125 seeds, lansoprazole, ibandronic acid, ibritumomab tiuxetan, ibrutinib, idarubicin, ifosfamide, imatinib, imiquimod, improsulfan, indisetron, incadronic acid, ingenol mebutate, inotuzumab ozogamicin, interferon alfa, interferon beta, interferon gamma, iobitridol, iobenguane (123I), iomeprol, ipilimumab, irinotecan, Itraconazole, ixabepilone, ixazomib, lanreotide, lansoprazole, lapatinib, lasocholine, lenalidomide, lenvatinib, lenograstim, lentinan, letrozole, leuprorelin, levamisole, levonorgestrel, levothyroxine sodium, lisuride, lobaplatin, lomustine, lonidamine, lutetium Lu 177 dotatate, masoprocol, medroxyprogesterone, megestrol, melarsoprol, melphalan, mepitiostane, mercaptopurine, mesna, methadone, methotrexate, methoxsalen, methylaminolevulinate, methylprednisolone, methyltestosterone, metirosine, midostaurin, mifamurtide, miltefosine, miriplatin, mitobronitol, mitoguazone, mitolactol, mitomycin, mitotane, mitoxantrone, mogamulizumab, molgramostim, mopidamol, morphine hydrochloride, morphine sulfate, mvasi, nabilone, nabiximols, nafarelin, naloxone+pentazocine, naltrexone, nartograstim, necitumumab, nedaplatin, nelarabine, neratinib, neridronic acid, netupitant/palonosetron, nivolumab, pentetreotide, nilotinib, nilutamide, nimorazole, nimotuzumab, nimustine, nintedanib, niraparib, nitracrine, nivolumab, obinutuzumab, octreotide, ofatumumab, olaparib, olaratumab, omacetaxine mepesuccinate, omeprazole, ondansetron, oprelvekin, orgotein, orilotimod, osimertinib, oxaliplatin, oxycodone, oxymetholone, ozogamicine, p53 gene therapy, paclitaxel, palbociclib, palifermin, palladium-103 seed, palonosetron, pamidronic acid, panitumumab, panobinostat, pantoprazole, pazopanib, pegaspargase, PEG-epoetin beta (methoxy PEG-epoetin beta), pembrolizumab, pegfilgrastim, peginterferon alfa-2b, pemetrexed, pentazocine, pentostatin, peplomycin, Perflubutane, perfosfamide, Pertuzumab, picibanil, pilocarpine, pirarubicin, pixantrone, plerixafor, plicamycin, poliglusam, polyestradiol phosphate, polyvinylpyrrolidone+sodium hyaluronate, polysaccharide-K, pomalidomide, ponatinib, porfimer sodium, pralatrexate, prednimustine, prednisone, procarbazine, procodazole, propranolol, quinagolide, rabeprazole, racotumomab, radium-223 chloride, radotinib, raloxifene, raltitrexed, ramosetron, ramucirumab, ranimustine, rasburicase, razoxane, refametinib, regorafenib, ribociclib, risedronic acid, rhenium-186 etidronate, rituximab, rolapitant, romidepsin, romiplostim, romurtide, rucaparib, samarium (153Sm) lexidronam, sargramostim, sarilumab, satumomab, secretin, siltuximab, sipuleucel-T, sizofiran, sobuzoxane, sodium glycididazole, sonidegib, sorafenib, stanozolol, streptozocin, sunitinib, talaporfin, talimogene laherparepvec, tamibarotene, tamoxifen, tapentadol, tasonermin, teceleukin, technetium (99mTc) nofetumomab merpentan, 99mTc-HYNIC-[Tyr3]-octreotide, tegafur, tegafur+gimeracil+oteracil, temoporfin, temozolomide, temsirolimus, teniposide, testosterone, tetrofosmin, thalidomide, thiotepa, thymalfasin, thyrotropin alfa, tioguanine, tisagenlecleucel, tislelizumab, tocilizumab, topotecan, toremifene, tositumomab, trabectedin, trametinib, tramadol, trastuzumab, trastuzumab emtansine, treosulfan, tretinoin, trifluridine+tipiracil, trilostane, triptorelin, trametinib, trofosfamide, thrombopoietin, tryptophan, ubenimex, valatinib, valrubicin, vandetanib, vapreotide, vemurafenib, vinblastine, vincristine, vindesine, vinflunine, vinorelbine, vismodegib, vorinostat, vorozole, yttrium-90 glass microspheres, zinostatin, zinostatin stimalamer, zoledronic acid, zorubicin.

The compounds of the invention can further be combined with other reagents targeting the immune system, such as immune checkpoint inhibitors, e.g. aPD-1/-L1 axis antagonists. PD-1, along with its ligands PD-L1 and PD-L2, function as negative regulators of T cell activation. DGKα suppresses immune cell function. PD-L1 is overexpressed in many cancers and overexpression of PD-1 often occurs concomitantly in tumor infiltrating T cells. This results in attenuation of T cell activation and evasion of immune surveillance, which contributes to impaired antitumor immune responses. (Keir M E et al. (2008) Annu. Rev. Immunol. 26:677).

In accordance with a further aspect, the present invention covers combinations comprising one or more of the compounds of general formula (I), as described herein, or stereoisomers, tautomers, N-oxides, hydrates, solvates, and salts thereof, particularly pharmaceutically acceptable salts thereof, or mixtures of same, and one or more immune checkpoint inhibitors. Preferably, the immune checkpoint inhibitor is a aPD-1/-L1 axis antagonist.

The compounds of the invention can further be combined with chimeric antigen receptor T cells (CAR-T cells), such as Axicabtagen-Ciloleucel or Tisagenlecleucel. The activity of CAR-T cells can be suppressed by the tumor micro environment (TME). Knock out of DGKα by techniques such as Crispr had been shown to enhance CAR-T cell activity in a suppressive TME (Mol. Cells 2018; 41(8): 717-723).

In accordance with a further aspect, the present invention covers combinations comprising one or more compounds of general formula (I), as described herein, or stereoisomers, tautomers, N-oxides, hydrates, solvates, and salts thereof, particularly pharmaceutically acceptable salts thereof, or mixtures of same, with chimeric antigen receptor T cells, (CAR-T cells), CAR-NKT cells or CAR-NK cells.

Preferably, the chimeric antigen receptor T cells (CAR-T cells) are Axicabtagen-Ciloleucel or Tisagenlecleucel.

The present invention further provides the use of the compounds according to the invention for expansion of T cells including CAR-T and tumor infiltrated lymphocytes ex-vivo. Inhibition of DGKα was shown to reactivate ex vivo treated T cells (Prinz et al. (2012) J. Immunol).

In accordance with a further aspect, the present invention covers compounds of general formula (I), as described herein, or stereoisomers, tautomers, N-oxides, hydrates, solvates, and salts thereof, particularly pharmaceutically acceptable salts thereof, or mixtures of same, for use in the expansion of T cells including CAR-T cells, CAR-NKT cells or CAR-NK cells and tumor infiltrated lymphocytes ex-vivo.

Hence, the present invention also relates to the use of the compounds according to the invention for the expansion of T cells, including CAR-T cell, CAR-NKT cells or CAR-NK cells and tumor infiltrated lymphocytes, ex-vivo.

The present invention also comprises an ex-vivo method for the expansion of T cells, including CAR-T cells, CAR-NKT cells or CAR-NK cells and tumor infiltrated lymphocytes, contacting said T cells with compounds according to the invention.

The compounds of the invention can further be combined with inhibitors of DGKζ, such as those inhibitors of DGKζ disclosed in WO2020/006016 and WO2020/006018. As DGKζ in T cells operates in a similar fashion as DGKα, a dual inhibition profoundly enhances T cell effector functions compared with cells with deletion of either DGK isoform alone or wild-type cells (Riese et al., Cancer Res 2013, 73(12), 3566).

Compounds of the present invention can be utilized to inhibit, block, reduce or decrease DGKα activity resulting in the modulation of dysregulated immune responses e.g. to block immunosuppression and increase immune cell activation and infiltration in the context of cancer and cancer immunotherapy that will eventually lead to reduction of tumour growth.

This method comprises administering to a mammal in need thereof, including a human, an amount of a compound of this invention, or a pharmaceutically acceptable salt, isomer, polymorph, metabolite, hydrate, solvate or ester thereof; which is effective to treat the disorder.

The present invention also provides methods of treating a variety of other disorders wherein DGKα is involved such as, but not limited to, disorders with dysregulated immune responses, inflammation, vaccination for infection & cancer, viral infections, obesity and diet-induced obesity, adiposity, metabolic disorders, fibrotic disorders, cardiac diseases and lymphoproliferative disorders.

These disorders have been well characterized in humans, but also exist with a similar etiology in other mammals, and can be treated by administering pharmaceutical compositions of the present invention.

In accordance with a further aspect, the present invention covers compounds of general formula (I), as described supra, or stereoisomers, tautomers, N-oxides, hydrates, solvates, and salts thereof, particularly pharmaceutically acceptable salts thereof, or mixtures of same, for use in the treatment or prophylaxis of diseases, in particular cancer or conditions with dysregulated immune responses or other disorders associated with aberrant DGKα signaling.

The pharmaceutical activity of the compounds according to the invention can be explained by their activity as DGKα inhibitors.

In accordance with a further aspect, the present invention covers the use of compounds of general formula (I), as described supra, or stereoisomers, tautomers, N-oxides, hydrates, solvates, and salts thereof, particularly pharmaceutically acceptable salts thereof, or mixtures of same, for the treatment or prophylaxis of diseases, in particular cancer or conditions with dysregulated immune responses or other disorders associated with aberrant DGKα signaling, particularly liquid and solid tumours.

In accordance with a further aspect, the present invention covers the compounds of general formula (I), as described supra, or stereoisomers, tautomers, N-oxides, hydrates, solvates, and salts thereof, particularly pharmaceutically acceptable salts thereof, or mixtures of same, for the use of treatment or prophylaxis of diseases, in particular cancer or conditions with dysregulated immune responses or other disorders associated with aberrant DGKα signaling, particularly liquid and solid tumours.

In accordance with a further aspect, the present invention covers the use of compounds of general formula (I), as described supra, or stereoisomers, tautomers, N-oxides, hydrates, solvates, and salts thereof, particularly pharmaceutically acceptable salts thereof, or mixtures of same, in a method of treatment or prophylaxis of diseases, in particular cancer or conditions with dysregulated immune responses or other disorders associated with aberrant DGKα signaling, particularly liquid and solid tumours.

In accordance with a further aspect, the present invention covers use of a compound of general formula (I), as described supra, or stereoisomers, tautomers, N-oxides, hydrates, solvates, and salts thereof, particularly pharmaceutically acceptable salts thereof, or mixtures of same, for the preparation of a pharmaceutical composition, preferably a medicament, for the prophylaxis or treatment of diseases, in particular cancer or conditions with dysregulated immune responses or other disorders associated with aberrant DGKα signaling, particularly liquid and solid tumours.

In accordance with a further aspect, the present invention covers a method of treatment or prophylaxis of diseases, in particular cancer or conditions with dysregulated immune responses or other disorders associated with aberrant DGKα signaling, particularly liquid and solid tumours, using an effective amount of a compound of general formula (I), as described supra, or stereoisomers, tautomers, N-oxides, hydrates, solvates, and salts thereof, particularly pharmaceutically acceptable salts thereof, or mixtures of same.

In accordance with a further aspect, the present invention covers pharmaceutical compositions, in particular a medicament, comprising a compound of general formula (I), as described supra, or a stereoisomer, a tautomer, an N-oxide, a hydrate, a solvate, a salt thereof, particularly a pharmaceutically acceptable salt, or a mixture of same, and one or more excipients), in particular one or more pharmaceutically acceptable excipient(s). Conventional procedures for preparing such pharmaceutical compositions in appropriate dosage forms can be utilized.

The present invention furthermore covers pharmaceutical compositions, in particular medicaments, which comprise at least one compound according to the invention, conventionally together with one or more pharmaceutically suitable excipients, and to their use for the above mentioned purposes.

It is possible for the compounds according to the invention to have systemic and/or local activity. For this purpose, they can be administered in a suitable manner, such as, for example, via the oral, parenteral, pulmonary, nasal, sublingual, lingual, buccal, rectal, vaginal, dermal, transdermal, conjunctival, otic route or as an implant or stent.

For these administration routes, it is possible for the compounds according to the invention to be administered in suitable administration forms.

For oral administration, it is possible to formulate the compounds according to the invention to dosage forms known in the art that deliver the compounds of the invention rapidly and/or in a modified manner, such as, for example, tablets (uncoated or coated tablets, for example with enteric or controlled release coatings that dissolve with a delay or are insoluble), orally-disintegrating tablets, films/wafers, films/lyophylisates, capsules (for example hard or soft gelatine capsules), sugar-coated tablets, granules, pellets, powders, emulsions, suspensions, aerosols or solutions. It is possible to incorporate the compounds according to the invention in crystalline and/or amorphised and/or dissolved form into said dosage forms.

Parenteral administration can be effected with avoidance of an absorption step (for example intravenous, intraarterial, intracardial, intraspinal or intralumbal) or with inclusion of absorption (for example intramuscular, subcutaneous, intracutaneous, percutaneous or intraperitoneal).

Administration forms which are suitable for parenteral administration are, inter alia, preparations for injection and infusion in the form of solutions, suspensions, emulsions, lyophylisates or sterile powders.

Examples which are suitable for other administration routes are pharmaceutical forms for inhalation [inter alia powder inhalers, nebulizers], nasal drops, nasal solutions, nasal sprays; tablets/films/wafers/capsules for lingual, sublingual or buccal administration; suppositories; eye drops, eye ointments, eye baths, ocular inserts, ear drops, ear sprays, ear powders, ear-rinses, ear tampons; vaginal capsules, aqueous suspensions (lotions, mixturae agitandae), lipophilic suspensions, emulsions, ointments, creams, transdermal therapeutic systems (such as, for example, patches), milk, pastes, foams, dusting powders, implants or stents.

The compounds according to the invention can be incorporated into the stated administration forms. This can be effected in a manner known per se by mixing with pharmaceutically suitable excipients. Pharmaceutically suitable excipients include, inter alia,
fillers and carriers (for example cellulose, microcrystalline cellulose (such as, for example, Avicel®), lactose, mannitol, starch, calcium phosphate (such as, for example, Di-Cafos®)),
ointment bases (for example petroleum jelly, paraffins, triglycerides, waxes, wool wax, wool wax alcohols, lanolin, hydrophilic ointment, polyethylene glycols),
bases for suppositories (for example polyethylene glycols, cacao butter, hard fat),
solvents (for example water, ethanol, isopropanol, glycerol, propylene glycol, medium chain-length triglycerides fatty oils, liquid polyethylene glycols, paraffins),
surfactants, emulsifiers, dispersants or wetters (for example sodium dodecyl sulfate), lecithin, phospholipids, fatty alcohols (such as, for example, Lanette®), sorbitan fatty acid esters (such as, for example, Span®), polyoxyethylene sorbitan fatty acid esters (such as, for example, Tween®), polyoxyethylene fatty acid glycerides (such as, for example, Cremophor®), polyoxethylene fatty acid esters, polyoxyethylene fatty alcohol ethers, glycerol fatty acid esters, poloxamers (such as, for example, Pluronic®),
buffers, acids and bases (for example phosphates, carbonates, citric acid, acetic acid, hydrochloric acid, sodium hydroxide solution, ammonium carbonate, trometamol, triethanolamine),
isotonicity agents (for example glucose, sodium chloride),
adsorbents (for example highly-disperse silicas),
viscosity-increasing agents, gel formers, thickeners and/or binders (for example polyvinylpyrrolidone, methylcellulose, hydroxypropylmethylcellulose, hydroxypropyl-cellulose, carboxymethylcellulose-sodium, starch, carbomers, polyacrylic acids (such as, for example, Carbopol®); alginates, gelatine),
disintegrants (for example modified starch, carboxymethylcellulose-sodium, sodium starch glycolate (such as, for example, Explotab®), cross-linked polyvinylpyrrolidone, croscarmellose-sodium (such as, for example, AcDiSol®)),
flow regulators, lubricants, glidants and mould release agents (for example magnesium stearate, stearic acid, talc, highly-disperse silicas (such as, for example, Aerosil®)),
coating materials (for example sugar, shellac) and film formers for films or diffusion membranes which dissolve rapidly or in a modified manner (for example polyvinylpyrrolidones (such as, for example, Kollidon®), polyvinyl alcohol, hydroxypropylmethylcellulose, hydroxypropylcellulose, ethylcellulose, hydroxypropyl-methylcellulose phthalate, cellulose acetate, cellulose acetate phthalate, polyacrylates, polymethacrylates such as, for example, Eudragit®)),
capsule materials (for example gelatine, hydroxypropylmethylcellulose),
synthetic polymers (for example polylactides, polyglycolides, polyacrylates, polymethacrylates (such as, for example, Eudragit®), polyvinylpyrrolidones (such as, for example, Kollidon®), polyvinyl alcohols, polyvinyl acetates, polyethylene oxides, polyethylene glycols and their copolymers and blockcopolymers),
plasticizers (for example polyethylene glycols, propylene glycol, glycerol, triacetine, triacetyl citrate, dibutyl phthalate),
penetration enhancers,
stabilisers (for example antioxidants such as, for example, ascorbic acid, ascorbyl palmitate, sodium ascorbate, butylhydroxyanisole, butylhydroxytoluene, propyl gallate),
preservatives (for example parabens, sorbic acid, thiomersal, benzalkonium chloride, chlorhexidine acetate, sodium benzoate),
colourants (for example inorganic pigments such as, for example, iron oxides, titanium dioxide),
flavourings, sweeteners, flavour- and/or odour-masking agents.

The present invention furthermore relates to a pharmaceutical composition which comprise at least one compound according to the invention, conventionally together with one or more pharmaceutically suitable excipient(s), and to their use according to the present invention.

In accordance with another aspect, the present invention covers pharmaceutical combinations, in particular medicaments, comprising at least one compound of general formula (I) of the present invention and at least one or more further active ingredients, in particular for the treatment and/or prophylaxis of cancer or conditions with dysregulated immune responses or other disorders associated with aberrant DGKα signaling, particularly liquid and solid tumours.

Particularly, the present invention covers a pharmaceutical combination, which comprises:
one or more first active ingredients, in particular compounds of general formula (I) as defined supra, and
one or more further active ingredients, in particular in particular immune checkpoint inhibitors.

The term “combination” in the present invention is used as known to persons skilled in the art, it being possible for said combination to be a fixed combination, a non-fixed combination or a kit-of-parts.

A “fixed combination” in the present invention is used as known to persons skilled in the art and is defined as a combination wherein, for example, a first active ingredient, such as one or more compounds of general formula (I) of the present invention, and a further active ingredient are present together in one unit dosage or in one single entity. One example of a “fixed combination” is a pharmaceutical composition wherein a first active ingredient and a further active ingredient are present in admixture for simultaneous administration, such as in a formulation. Another example of a “fixed combination” is a pharmaceutical combination wherein a first active ingredient and a further active ingredient are present in one unit without being in admixture.

A non-fixed combination or “kit-of-parts” in the present invention is used as known to persons skilled in the art and is defined as a combination wherein a first active ingredient and a further active ingredient are present in more than one unit. One example of a non-fixed combination or kit-of-parts is a combination wherein the first active ingredient and the further active ingredient are present separately. It is possible for the components of the non-fixed combination or kit-of-parts to be administered separately, sequentially, simultaneously, concurrently or chronologically staggered.

Based upon standard laboratory techniques known to evaluate compounds useful for the treatment of cancer or conditions with dysregulated immune responses or other disorders associated with aberrant DGKα signaling, by standard toxicity tests and by standard pharmacological assays for the determination of treatment of the conditions identified above in mammals, and by comparison of these results with the results of known active ingredients or medicaments that are used to treat these conditions, the effective dosage of the compounds of the present invention can readily be determined for treatment of each desired indication. The amount of the active ingredient to be administered in the treatment of one of these conditions can vary widely according to such considerations as the particular compound and dosage unit employed, the mode of administration, the period of treatment, the age and sex of the patient treated, and the nature and extent of the condition treated.

The total amount of the active ingredient to be administered will generally range from about 0.001 mg/kg to about 200 mg/kg body weight per day, and preferably from about 0.01 mg/kg to about 20 mg/kg body weight per day. Clinically useful dosing schedules will range from one to three times a day dosing to once every four weeks dosing. In addition, it is possible for “drug holidays”, in which a patient is not dosed with a drug for a certain period of time, to be beneficial to the overall balance between pharmacological effect and tolerability. It is possible for a unit dosage to contain from about 0.5 mg to about 1500 mg of active ingredient, and can be administered one or more times per day or less than once a day. The average daily dosage for administration by injection, including intravenous, intramuscular, subcutaneous and parenteral injections, and use of infusion techniques will preferably be from 0.01 to 200 mg/kg of total body weight. The average daily rectal dosage regimen will preferably be from 0.01 to 200 mg/kg of total body weight. The average daily vaginal dosage regimen will preferably be from 0.01 to 200 mg/kg of total body weight. The average daily topical dosage regimen will preferably be from 0.1 to 200 mg administered between one to four times daily. The transdermal concentration will preferably be that required to maintain a daily dose of from 0.01 to 200 mg/kg. The average daily inhalation dosage regimen will preferably be from 0.01 to 100 mg/kg of total body weight.

Of course the specific initial and continuing dosage regimen for each patient will vary according to the nature and severity of the condition as determined by the attending diagnostician, the activity of the specific compound employed, the age and general condition of the patient, time of administration, route of administration, rate of excretion of the drug, drug combinations, and the like. The desired mode of treatment and number of doses of a compound of the present invention or a pharmaceutically acceptable salt or ester or composition thereof can be ascertained by those skilled in the art using conventional treatment tests.

Syntheses of Compounds

The compounds according to the invention of general formula (I) can be prepared according to the following schemes 1-16. The schemes and procedures described below illustrate synthetic routes to the compounds of general formula (I) of the invention and are not intended to be limiting. It is clear to the person skilled in the art that the order of transformations as exemplified in schemes 1-16 can be modified in various ways. The order of transformations exemplified in these schemes is therefore not intended to be limiting. In addition, interconversion of any of the substituents, R¹, R², R³, R⁴, R⁵, R⁶, R⁷, R⁸ or X can be achieved before and/or after the exemplified transformations. These modifications can be such as the introduction of protecting groups, cleavage of protecting groups, reduction or oxidation of functional groups, halogenation, metalation or substitution known to the person skilled in the art. These transformations include those which introduce a functionality which allows for further interconversion of substituents. Appropriate protecting groups and their introduction and cleavage are well-known to the person skilled in the art (see for example T. W. Greene and P. G. M. Wuts in Protective Groups in Organic Synthesis, 4^{th} edition, Wiley 2006). Specific examples are described in the subsequent paragraphs.

Isatoic anhydrides 1 are widely available from commercial suppliers or described in the literature. For example the isatoic anhydrides 1 can be prepared from 2-aminobenzoic acids 2 (in analogy to the procedure in Tetrahedron Lett. 2014, 55, 3607-3609) using triphosgene in an organic solvent such as THF or 1,4-dioxane or (in analogy to the procedure in Tetrahedron Lett. 2013, 54, 6897-6899) using di-tert-butyl dicarbonate and a base such as sodium hydroxide followed by treatment with 2-chloromethylpyridinium iodide and subsequent acidic workup (Scheme 1).

Alternatively, preparation of the isatoic anhydrides 1 can also be achieved (for example in analogy to the procedure in J. Org. Chem. 2014, 79, 4196-4200) using Pd-catalyzed oxidative double carbonylation of o-iodoanilines 3.

The obtained isatoic anhydrides 1 can then be alkylated at the nitrogen to obtain compounds of the general formula 4. Typically an alkylating agent such as for example an alkylbromide, alkyliodide or alkylsulfonate, a base such as disopropylethylamine, potassium carbonate or potassium tert-butoxide in an organic solvent is used.

Alternatively the alkylated isatoic anhydrides 4 can be prepared directly from secondary anilines 5 (in analogy to the procedure in Tetrahedron Lett. 2014, 55, 3607-3609) using triphosgene in an organic solvent such as THF or 1,4-dioxane or (in analogy to the procedure in Tetrahedron Lett. 2013, 54, 6897-6899) using di-tert-butyl dicarbonate and a base such as sodium hydroxide followed by treatment with 2-chloromethylpyridinium iodide and subsequent acidic workup.

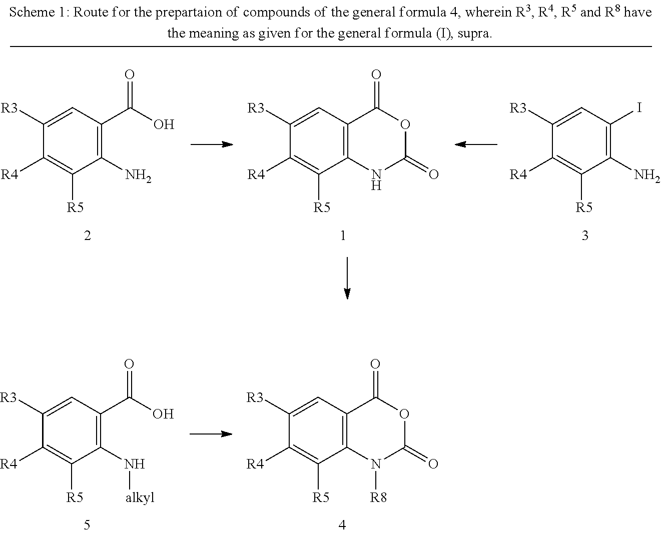

Isatoic anhydrides 4 can be converted to the corresponding quinolones 7 using ethyl acetate derivatives 6 such as for example ethylcyano acetate (for R¹=CN), a base such as for example triethylamine in an organic solvent such as for example THF (Scheme 2).

Hydroxy quinolones 7 can be converted to the corresponding halides 8 using for example phosphoryl chloride (Y=chloro) or phosphoryl bromide (Y=bromo).

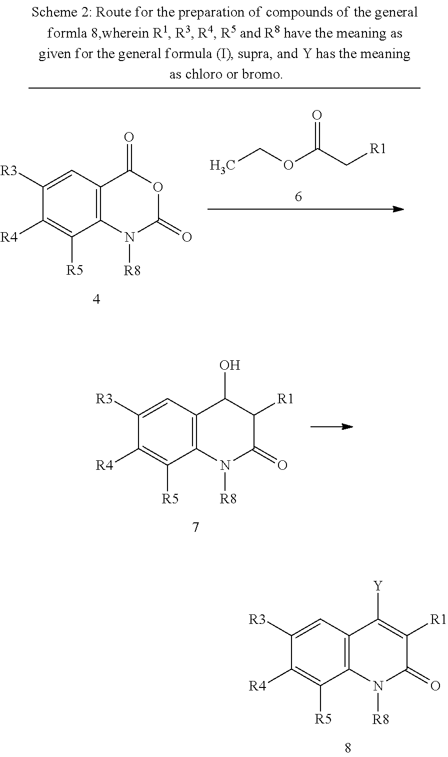

Halides of the general formula 8 can be reacted with amines 9 to yield compounds of the general formula 10 (Scheme 3). Typically the reaction is performed in an organic solvent such as for example isopropanol and a base such as for example diisopropylethylamine or triethylamine. Many amines of the general formula 9 are commercially available or described in the literature.

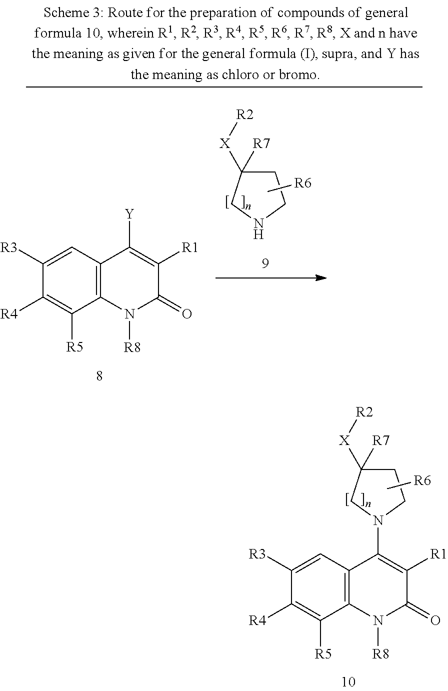

Nitriles of the general formula 11 can be converted to the amides of the general formula 12 (Scheme 4). Typically the reaction is performed with palladium(II)acetate and acetaldoxime in an organic solvent such as for example ethanol (see for example J. Med. Chem. 2016, 59, 6281ff, Degorce et al.).

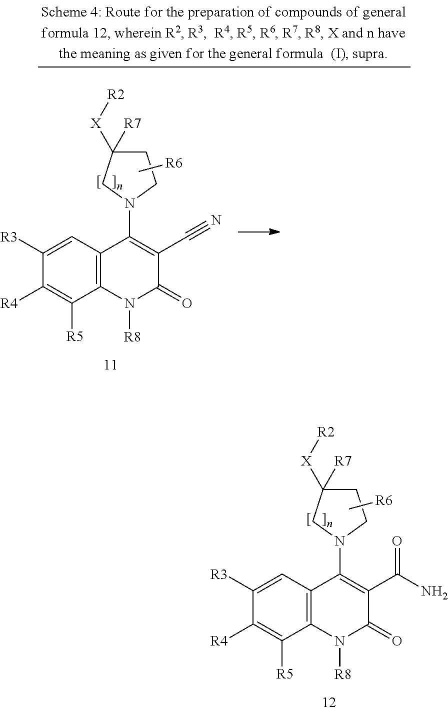

Alcohols of the general formula 13 can be converted to the ethers of the general formula 15 (Scheme 5). The reaction can for example be perfomed under Mitsunobu conditions known to the skilled person. Typical conditions are triphenylphosphine and diisopropyl azodicarboxylate in an organic solvent (see for example Org. Chem. Front. 2015, 2, 739-752).

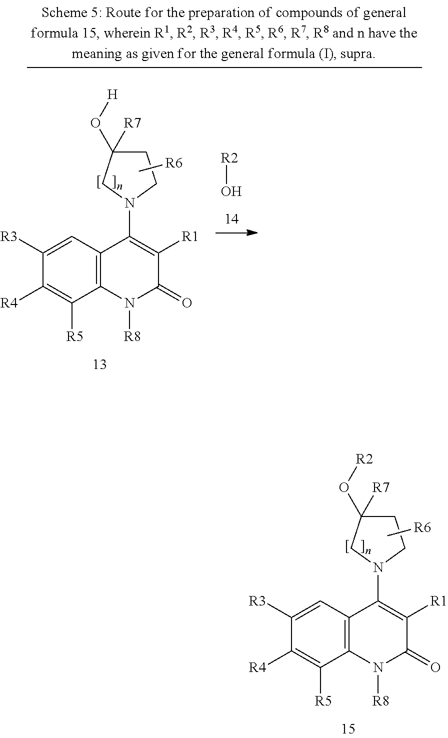

Isatoic anhydrides 4 can be converted to the corresponding quinolones 16 using diethylmalonate, a base such as for example triethylamine in an organic solvent such as for example THF (Scheme 6). Hydroxy quinolones 16 can be converted to the corresponding halides 17 using for example phosphoryl chloride (Y=chloro) or boron tribromide (Y=bromo). Halides of the general formula 17 can be reacted with amines 9 to yield compounds of the general formula 18. Typically the reaction is performed in an organic solvent such as for example isopropanol and with a base such as for example diisopropylethylamine.

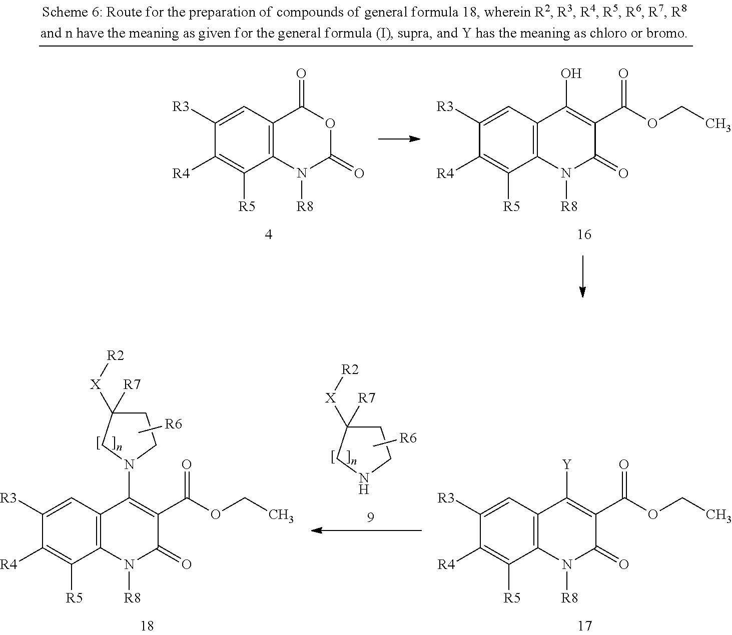

Esters of the general formula 18 can be converted to the corresponding carboxylic acids 19 using classical ester hydrolysis conditions (Scheme 7). Typically lithium hydroxide, sodium hydroxide or porassium hydroxide water/ethanol/THF at elevated temperatures is used for this reaction. The carboxylic acids of the general formula 19 and amines of general formula 20 can be converted to the corresponding amides 21 using standard amide forming reaction known to the person skilled in the art. For a review see for example Chem. Rev. 2011, 111, 6557-6602. Amines 20 are commercially available or described in the literature.

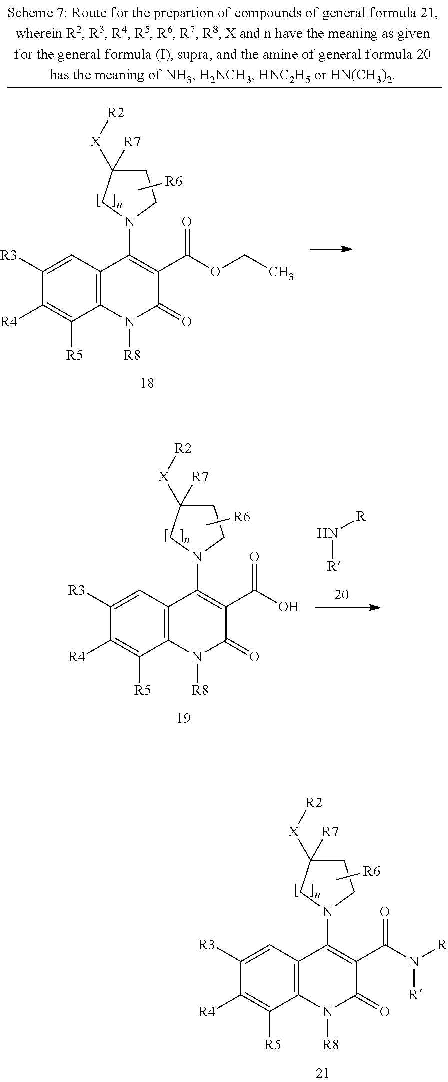

Ethers of the general formula 24 can be prepared from corresponding alcohols 22 via various nucleophilic substitution reactions followed by removal of the tertbutyloxycarbonyl protecting group under acidic conditions (for example with trifluoroacetic acid or hydrochloric acid) known to the person skilled in the art. Instead of a tertbutyloxycarbonyl other suitable protecting groups can be used for this sequence known to the skilled person (see for example P. G. M. Wuts and T. W. Greene in “Protective Groups in Organic Synthesis”, 4^{th} edition, Wiley 2006). Compounds of general formula 22 and 14 are commercially available or described in the literature.

Ethers of the general formula 23 can be prepared in one step from the corresponding alcohols 22 and 14 under Mitsunobu conditions known to the skilled person (Scheme 8). Typical conditions are triphenylphosphine and diisopropyl azodicarboxylate or diethyl azodicarboxylate in an organic solvent (see for example Org. Chem. Front. 2015, 2, 739-752). In a second step tertbutyloxycarbonyl protected ethers 23 are converted to the corresponding free amines 24 by treatment with trifluoroacetic acid or concentrated hydrochloric acid.

In an alternative procedure (Scheme 8) the alcohols of general formula 22 can be converted to the corresponding halides 25. For Hal=bromo typically carbon tetrabromide, triphenylphosphine and 1H-imidazole in an organic solvent such as for example dichloromethane is used (see for example Chemistry—A European Journal, 2015, 21, 12797). For Hal=iodo typically iodine, triphenylphosphine and 1H-imidazole in an organic solvent such as for example tetrahydrofuran is used (see for example Journal of Organic Chemistry, 2004, 69, 5120-5123). In a subsequent step halides 25 can be reacted with alcohols 14 to the corresponding ethers 23. Typically a base such as for example potassium carbonate in an organic solvent such as for example N,N-dimethyl-formamide can be used (see for example European Journal of Medicinal Chemistry, 2016, 108, 655-662).

In an alternative procedure (Scheme 8) alcohols 22 can be converted to the corresponding sulfonates 26. The sulfonate leaving groups are known to the skilled person (LG-O has the meaning as a sulfonyloxy leaving group such as for example (methylsulfonyl)oxy, [(trifluoromethyl)sulfonyl]oxy or [(4-methylphenyl)sulfonyl]oxy). For the conversion of 22 to 26 typically a sulfonylation reagent such as for example trifluoromethanesulfonic anhydride, 4-methylbenzenesulfonyl chloride or methanesulfonylchloride, a base such as for example triethylamine in an organic solvent such as for example dichloromethane can be used (see for example Journal of Medicinal Chemistry, 2014, 74, 562-573). In a subsequent step sulfonates 26 can be reacted with alcohols 14 to the corresponding ethers 23. Typically a base such as for example potassium carbonate in an organic solvent such as for example N,N-dimethyl-formamide can be used (see for example Journal of Medicinal Chemistry, 2016, 59, 3964-3979).

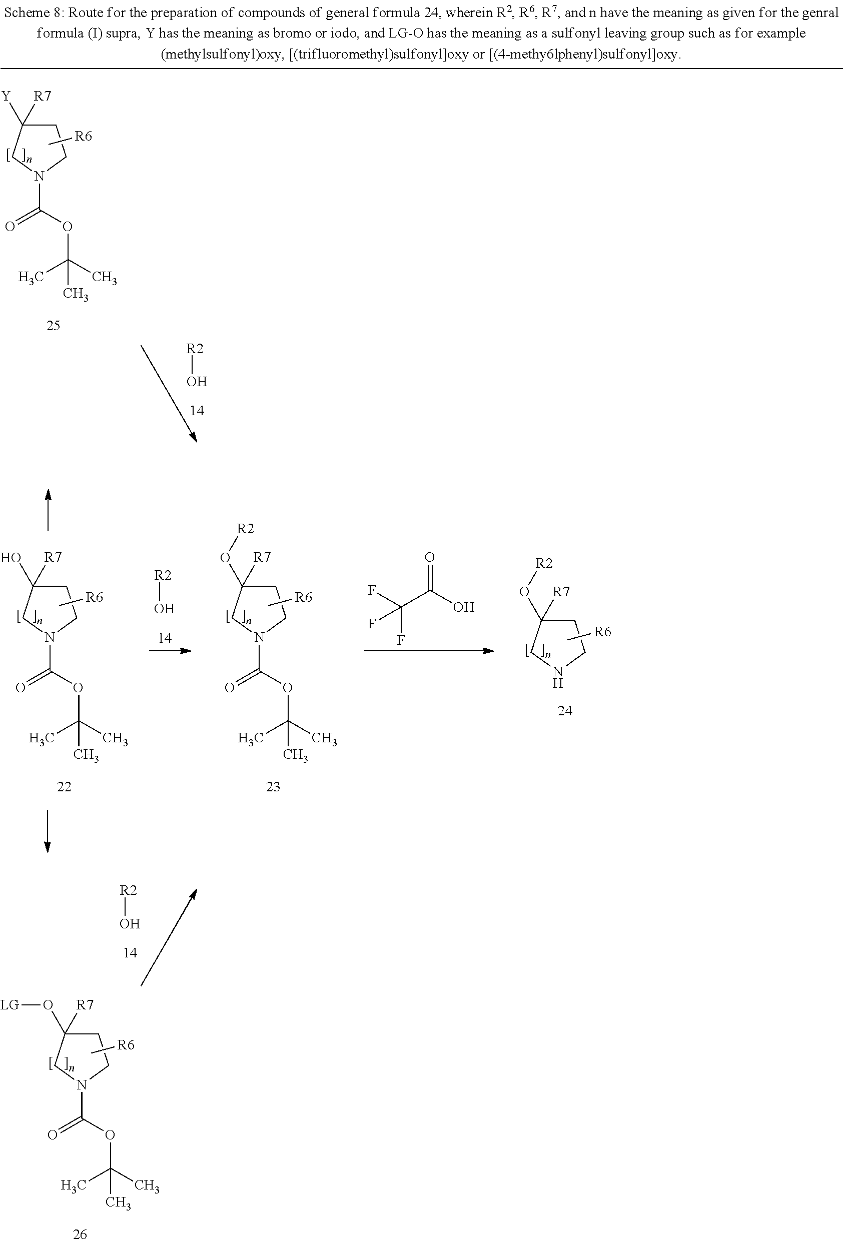

Thioethers of the general formula 29 can be prepared from corresponding alcohols 22 via various nucleophilic substitution reactions followed by removal of the tertbutyloxycarbonyl protecting group under acidic conditions (for example with trifluoroacetic acid or hydrochloric acid) known to the person skilled in the art. Instead of a tertbutyloxycarbonyl other suitable protecting groups can be used for this sequence known to the skilled person (see for example P. G. M. Wuts and T. W. Greene in “Protective Groups in Organic Synthesis”, 4^{th} edition, Wiley 2006). Compounds of general formula 22 and 27 are commercially available or described in the literature.

Thioethers of the general formula 28 can be prepared in one step from the corresponding alcohols 22 and thiols 27 under Mitsunobu conditions known to the skilled person (Scheme 9). Typical conditions are triphenylphosphine and diisopropyl azodicarboxylate or diethyl azodicarboxylate in an organic solvent (see for example Bioorganic and Medicinal Chemistry Letters, 2015, 25, 529-541). In a second step tertbutyloxycarbonyl protected thioethers 28 are converted to the corresponding free amines 29 by treatment with trifluoroacetic acid or concentrated hydrochloric acid.

Alternatively halides 25 can be reacted with thiols 27 to the corresponding thioethers 28 (Scheme 9). Typically a base such as for example potassium carbonate in an organic solvent such as for example N,N-dimethyl-formamide can be used (see for example WO2010/42867).

Alternatively (Scheme 9) sulfonates 26 can be reacted with thiols 27 to the corresponding thioethers 28. Typically a base such as for example potassium carbonate in an organic solvent such as for example N,N-dimethyl-formamide can be used (see for example Journal of Medicinal Chemistry, 2006, 49, 2784-2793).

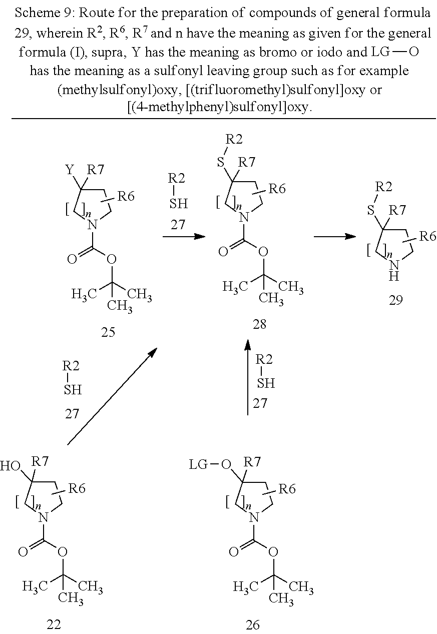

Ethers of the general formula 32 can be prepared from the corresponding alcohols 22 via various alkylation reactions followed by removal of the tertbutyloxycarbonyl protecting group (in 31) under acidic conditions (for example with trifluoroacetic acid or hydrochloric acid) known to the person skilled in the art. Instead of a tertbutyloxycarbonyl other suitable protecting groups can be used for this sequence known to the skilled person (see for example P. G. M. Wuts and T. W. Greene in “Protective Groups in Organic Synthesis”, 4^{th} edition, Wiley 2006). Compounds of general formula 22 and 30 are commercially available or described in the literature.

Ethers of the general formula 31 can be prepared in one step from the corresponding alcohols 22 and halides 30 under alkylating conditions known to the skilled person (Scheme 10). Typical conditions are for example a base such as sodium carbonate with or without potassium iodide in an organic solvent such as 1,4-dioxane or THF (see for example: Bioorganic and Medicinal Chemistry, 2007, 15, 6596-6607).

Typical reaction conditions for the compounds of the general formula 32 from the Boc protected amines 31 are for example trifluoroacetic acid or hydrochloric acid and are known to the person skilled in the art.

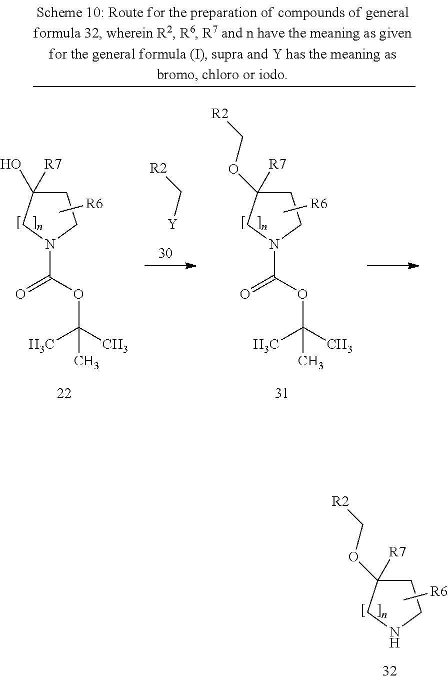

Ethers of the general formula 37 can be prepared from corresponding alcohols 33 via various nucleophilic substitution reactions followed by removal of the tertbutyloxycarbonyl protecting group under acidic conditions (for example with trifluoroacetic acid or hydrochloric acid) known to the person skilled in the art. Instead of a tertbutyloxycarbonyl other suitable protecting groups can be used for this sequence known to the skilled person (see for example P. G. M. Wuts and T. W. Greene in “Protective Groups in Organic Synthesis”, 4^{th} edition, Wiley 2006). Compounds of general formula 33 and 14 are commercially available or described in the literature.

Ethers of the general formula 36 can be prepared in one step from the corresponding alcohols 33 and 14 under Mitsunobu conditions known to the skilled person (Scheme 11). Typical conditions are triphenylphosphine and diisopropyl azodicarboxylate or diethyl azodicarboxylate in an organic solvent (see for example Org. Chem. Front. 2015, 2, 739-752). In a second step tertbutyloxycarbonyl protected ethers 36 are converted to the corresponding free amines 37 for example by treatment with trifluoroacetic acid or concentrated hydrochloric acid.

In an alternative procedure the alcohols of general formula 33 can be converted to the corresponding halides 34. For Y=bromo typically carbon tetrabromide, triphenylphosphine and 1H-imidazole in an organic solvent such as for example dichloromethane is used (see for example Chemistry—A European Journal, 2015, 21, 12797). For Y=iodo typically iodine, triphenylphosphine and 1H-imidazole in an organic solvent such as for example tetrahydrofuran is used (see for example Journal of Organic Chemistry, 2004, 69, 5120-5123). In a subsequent step halides 34 can be reacted with alcohols 14 to the corresponding ethers 36. Typically a base such as for example potassium carbonate in an organic solvent such as for example N,N-dimethyl-formamide can be used (see for example European Journal of Medicinal Chemistry, 2016, 108, 655-662).

In an alternative procedure alcohols 33 can be converted to the corresponding sulfonates 35. The sulfonate leaving groups are known to the skilled person (LG-O has the meaning as a sulfonyl leaving group such as for example (methylsulfonyl)oxy, [(trifluoromethyl)sulfonyl]oxy or [(4-methylphenyl)sulfonyl]oxy). For the conversion of 33 to 35 typically a sulfonylation reagent such as for example trifluoromethanesulfonic anhydride, 4-methylbenzenesulfonyl chloride or methanesulfonylchloride, a base such as for example triethylamine in an organic solvent such as for example dichloromethane can be used (see for example Journal of Medicinal Chemistry, 2014, 74, 562-573). In a subsequent step sulfonates 35 can be reacted with alcohols 14 to the corresponding ethers 36. Typically a base such as for example potassium carbonate in an organic solvent such as for example N,N-dimethyl-formamide can be used (see for example Journal of Medicinal Chemistry, 2016, 59, 3964-3979).

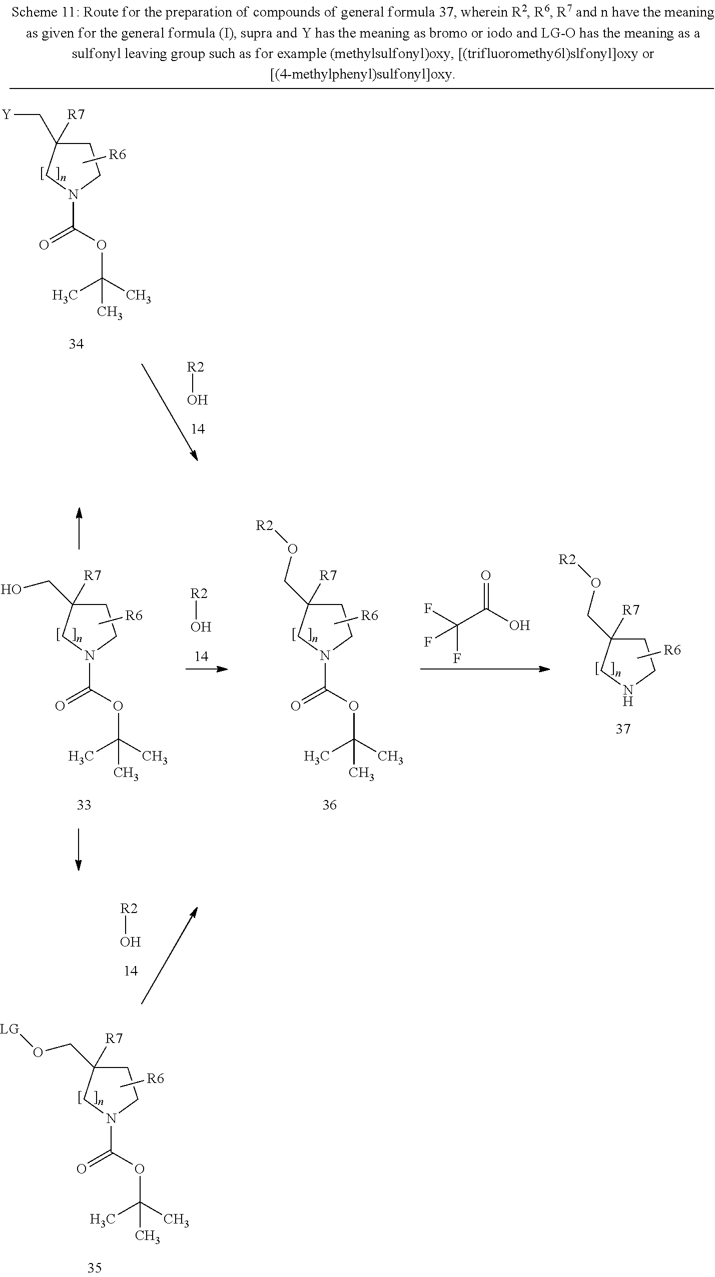

Compounds of the general formula 40 can be prepared from corresponding halides 34 via organometallic cross coupling reactions followed by removal of the tertbutyloxycarbonyl protecting group under acidic conditions (for example with trifluoroacetic acid or hydrochloric acid) known to the person skilled in the art. Instead of a tertbutyloxycarbonyl other suitable protecting groups can be used for this sequence known to the skilled person (see for example P. G. M. Wuts and T. W. Greene in “Protective Groups in Organic Synthesis”, 4^{th} edition, Wiley 2006). Compounds of general formula 38 are commercially available or described in the literature.

The organometallic cross coupling reaction of compound of the general formula 34 and boronic acids 38 is known to the person skilled in the art Scheme 12). Typically a metal catalyst such as for example nickel(II)iodide and a ligand such as trans-2-aminocyclohexanol and a base such as sodium bis(trimethylsilyl)amide in an organic solvent is used (see for example WO2014071363 or Angew. Chem. Int. Ed. 2009, 48, 2656-2670).

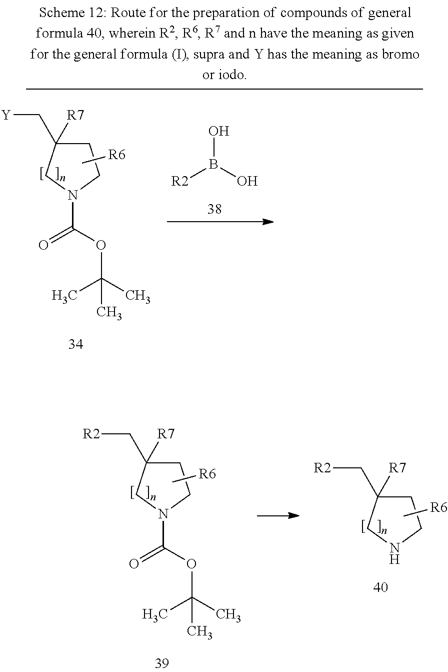

Alternatively compounds of the general formula 39 can be formed from compounds of the general formula 34 with Y=Br by a light promoted, nickel catalysed reaction as described in J. Am. Chem. Soc. 2016, 138, 8084-8087 and Org. Lett. 2016, 18, 4012, and known to one skilled in the art (Scheme 13). Preferentially, compounds of general formula 34 with Y=Br are reacted with bromides of the general formula 41 in the presence of a photoredox catalyst such as Ir(4′,6′-dF-5-CF3-ppy)2(4,4′-dtbbpy)PF6, a nickel precatalyst such as nickel II chloride dimethoxyethane adduct, and a ligand such as 4,4′-di-tert-butyl-2,2′-bipyridine, with a base such as sodium carbonate, 2,6-dimethoxypyridine, or lithium carbonate, with additives such as tris(trimehylsilyl)silane, in a solvent or solvent mixture such as dimethoxyethane, N,N-dimethylacetamide/trifluorotoluene or 1,3-dimethyl-2-imidazolidinone/trifluorotoluene, irradiated with light generated by two 40 W Kessil LED aquarium lights, at a temperature between 0° C. and the boiling point of the respective solvent. Ideally the reaction is performed between room temperature and 35° C. to afford compounds of general formula 39.

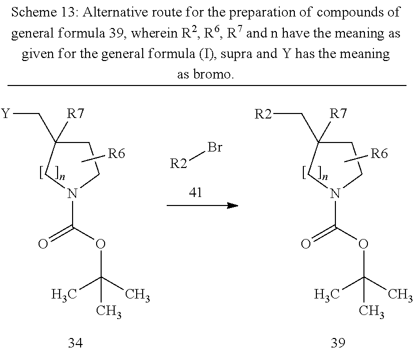

Compounds of the general formula 47 can be prepared from the corresponding nitriles 42 via reduction to the corresponding aldehydes 43 followed by a Corey-Fuchs reaction furnishes compounds of general formula 44 (Scheme 14). Subsequent Sonogashira reaction with bromides of the general formula 41 followed by hydrogenation of alkines 45 gives rise to compounds of the general formula 46. Removal of the tertbutyloxycarbonyl protecting group under acidic conditions (for example with trifluoroacetic acid or hydrochloric acid) furnishes compounds of general formula 47. Instead of a tertbutyloxycarbonyl other suitable protecting groups can be used for this sequence known to the skilled person (see for example P. G. M. Wuts and T. W. Greene in “Protective Groups in Organic Synthesis”, 4^{th} edition, Wiley 2006). All reactions of this sequence are known to the person skilled in the art. Compounds of the general formula 41 and 42 are commercially available or described in the literature.

Typical reaction conditions for the conversion of nitriles 42 to aldehydes 43 are a reducing agent such as diisobutylaluminium hydride in an organic solvent such as toluene (see for example Tetrahedron Letters, 2011, 52, 6058-6060).

Typical reaction conditions for the conversion of aldehydes 43 to alkynes 44 are known to the skilled person. For example, Corey-Fuchs reaction conditions such as (1-diazo-2-oxo-propyl)-phosphonic acid dimethyl ester, a base such as potassium carbonate in an organic solvent such as methanol can be used (see for example Journal of the American Chemical Society, 2003, 125, 3714-3715).

Typical reaction conditions for the conversion of alkynes 44 to alkynes 45 are known to the skilled person. For example, Sonogashira reaction conditions such as copper(I)iodide, a base such as cesium carbonate and/or N-ethyl-N,N-diisopropylamine, a palladium catalyst such as bis-triphenylphosphine-palladium(II)chloride in an organic solvent such as diethylene glycol dimethyl ether can be used (see for example procedure in WO2004/37796).

Typical hydrogenation conditions for the conversion of alkynes 45 to alkanes 46 are known to the skilled person. For example, hydrogen, a catalyst such as 10% palladium on activated carbon in an organic solvent such as for example methanol or ethanol (see for example procedure in WO2013/39802).

Typical reaction conditions for the deprotection of the tertbutyloxycarbonyl protecting group in compounds of the general formula 46 are an acid such as for example trifluoroacetic acid or hydrochloric acid giving rise to compound of the general formula 47.

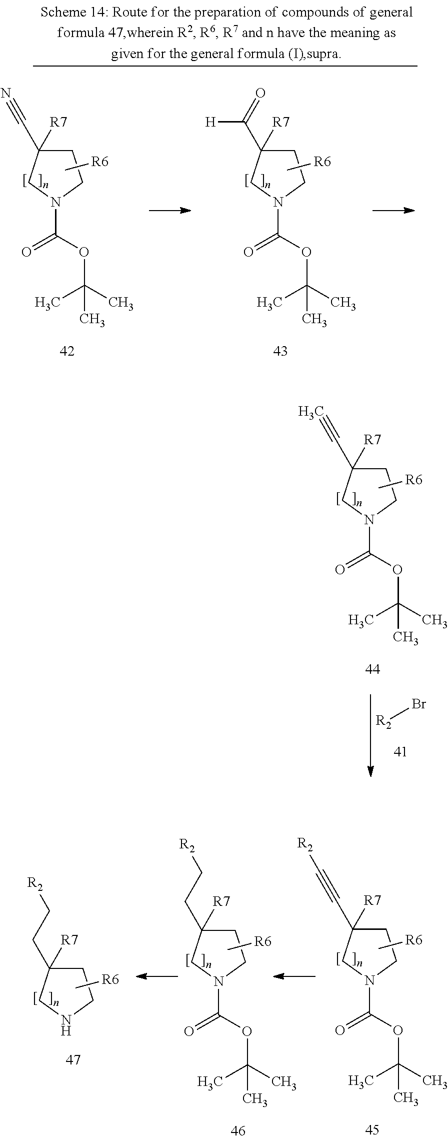

Alternatively compounds of the general formula 43 can be prepared from the corresponding esters 48 (with R=methyl, ethyl or tert-butyl) via reduction to the corresponding alcohols of general formula 49 and subsequent oxidation to the corresponding aldehydes of general formula 43 (Scheme 15). All reactions of this sequence are known to the person skilled in the art. Compounds of the general formula 48 are commercially available or described in the literature.

The conversion of the esters 48 to the aldehydes 43 is typically perfomed in a two-step procedure. In the first step the esters 48 are reduced to the corresponding alcohols of general formula 49. Typical reaction conditions are a reducing agent such as for example LiAlH₄/diethyl ether in an organic solvent such as for example THF or diethylether (see for example Journal of Medicinal Chemistry, 1994, 37, 113-124). In the second step the alcohols of the general formula 49 are oxidized to the corresponding aldehydes 43. Typical reaction conditions are for example Swern oxidation conditions such as DMSO, oxalyl chloride, a base such as trimethylamine in an organic solvent such as dichloromethane (see for example Journal of Medicinal Chemistry, 1994, 37, 113-124). Alternative reduction or oxidation conditions are known to the person skilled in the art.

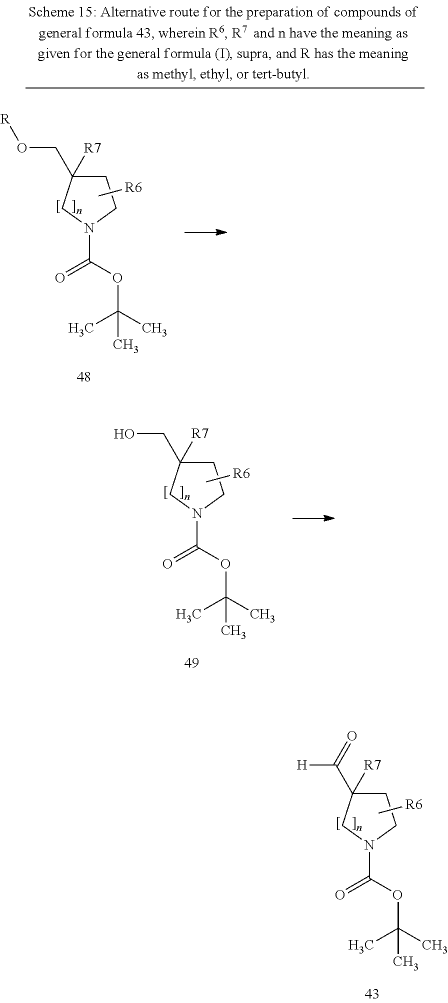

Benzoxazoles of the general formula 52 can be prepared from carboxylic acids 50 and 2-aminophenols 51 via a condensation reaction known to the one skilled in the art (Scheme 16). For example, the carboxylic acid 50 and the 2-aminophenols 51 can be reacted in polyphosphoric acid at elevated temperature. Compounds of general formula 50 and 51 are commercially available or described in the literature.

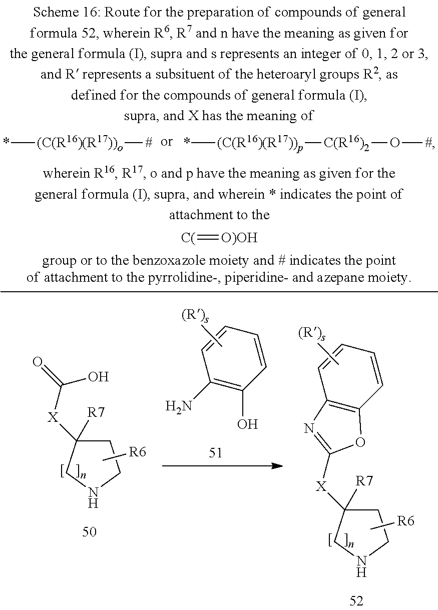

In accordance with a second aspect, the present invention covers methods of preparing compounds of general formula (I), said methods comprising the step of allowing an intermediate compound of general formula (II)

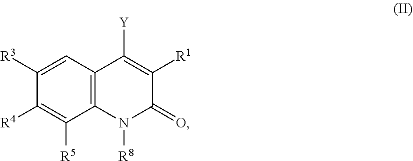

in which R¹, R³, R⁴, R⁵ and R⁸ are as defined for the compound of general formula (I) as defined supra, and Y has the meaning of chloro or bromo,
to react with a compound of general formula (III)

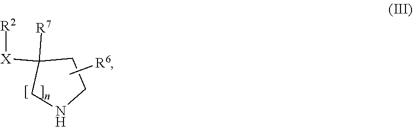

in which R², R⁶, R⁷, X and n are as defined for the compound of general formula (I) as defined supra,
thereby giving a compound of general formula (I)

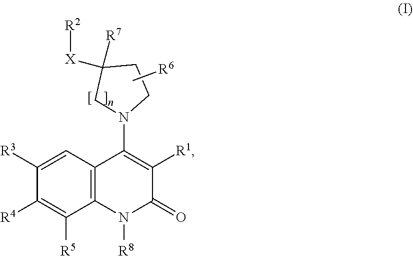

in which R¹, R², R³, R⁴, R⁵, R⁶, R⁷, R⁸, X and n are as defined supra.

In accordance with a second embodiment of the second aspect, the present invention covers methods of preparing compounds of general formula (I-b), which are compounds of general formula (I) in which R², R³, R⁴, R⁵, R⁶, R⁷, R⁸, X and n are as defined for the compound of general formula (I) as defined supra, and R¹ represents a carbamoyl group, said methods comprising the step of allowing a compound of general formula (I-a)

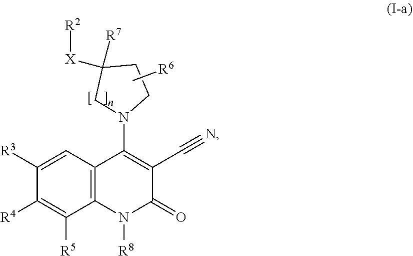

which is a compound of general formula (I) in which R², R³, R⁴, R⁵, R⁶, R⁷, R⁸, X and n are as defined for the compound of general formula (I) as defined supra, and R¹ represents a cyano group,
to react with palladium(II)acetate and acetaldoxime,
thereby giving a compound of general formula (I-b)

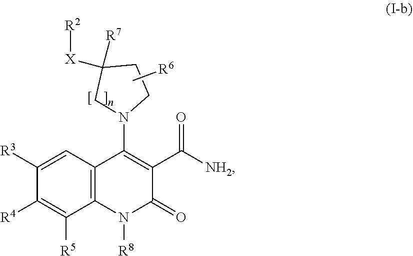

in which R², R³, R⁴, R⁵, R⁶, R⁷, R⁸, X and n are as defined supra, and R¹ represents a carbamoyl group.

In accordance with a third embodiment of the second aspect, the present invention covers methods of preparing compounds of general formula (I-c), which are compounds of general formula (I) in which R¹, R², R³, R⁴, R⁵, R⁶, R⁷, R⁸ and n are as defined for the compound of general formula (I) as defined supra, and X represents an O atom, said methods comprising the step of allowing an intermediate compound of general formula (IV)

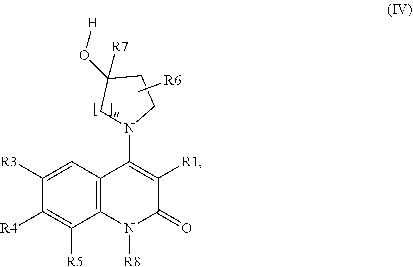

in which R¹, R³, R⁴, R⁵, R⁶, R⁷, R⁸ and n are as defined for the compound of general formula (I) as defined supra,
to react with a compound of general formula (V):

R²—OH (V),

in which R² is as defined for the compound of general formula (I) as defined supra, in the presence of triphenylphosphine and diisopropyl azodicarboxylate, thereby giving a compound of general formula (I-c)

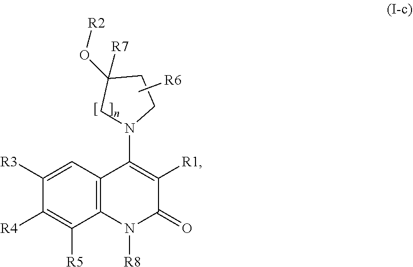

in which R¹, R², R³, R⁴, R⁵, R⁶, R⁷, R⁸ and n are as defined supra, and X represents an O atom.

In accordance with a fourth embodiment of the second aspect, the present invention covers methods of preparing compounds of general formula (I-e), which are compounds of general formula (I) in which R², R³, R⁴, R⁵, R⁶, R⁷, R⁸, X and n are as defined for the compound of general formula (I) as defined supra, and R¹ represents a —C(═O)NH₂, —C(═O)N(H)CH₃, —C(═O)N(H)C₂H₅ or —C(═O)N(CH₃)₂ group, said methods comprising the step of allowing a compound of general formula (I-d)

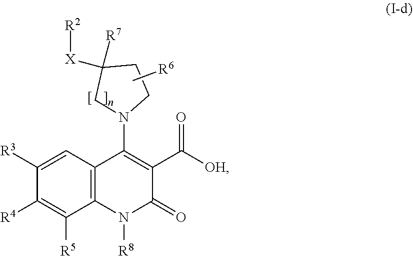

which is a compound of general formula (I) in which R², R³, R⁴, R⁵, R⁶, R⁷, R⁸, X and n are as defined for the compound of general formula (I) as defined supra, and R¹ represents a carboxyl group, to react with a compound of general formula (VI)

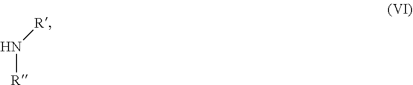

which compound is NH₃, H₂NCH₃, H₂NCH₂CH₃ or HN(CH₃)₂, or salts thereof, thereby giving a compound of general formula (I-e)

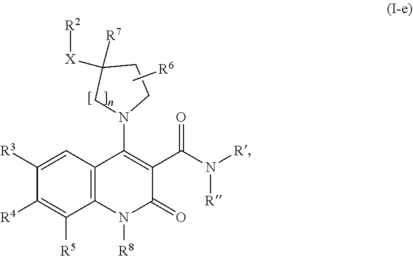

which is a compound of general formula (I) in which R², R³, R⁴, R⁵, R⁶, R⁷, R⁸, X and n are as defined supra, and R¹ represents a group —C(═O)NH₂, —C(═O)N(H)CH₃, —C(═O)N(H)C₂H₅ or —C(═O)N(CH₃)₂.

In accordance with a third aspect, the present invention covers methods of preparing compounds of general formula (I), said methods comprising the step of allowing an intermediate compound of general formula (II)

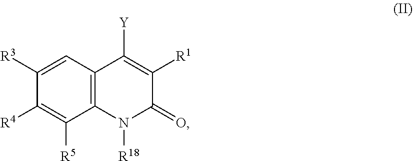

in which R¹, R³, R⁴, R⁵ and R⁸ are as defined for the compound of general formula (I) as defined supra, and Y has the meaning of chloro or bromo,
to react with a compound of general formula (III)

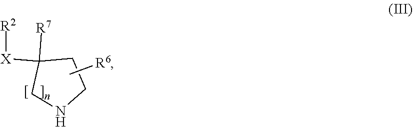

in which R², R⁶, R⁷, X and n are as defined for the compound of general formula (I) as defined supra,
thereby giving a compound of general formula (I)

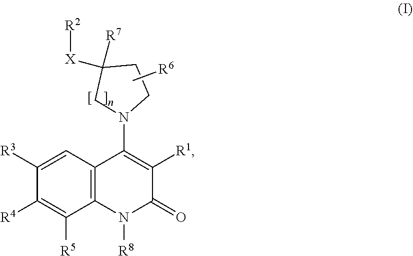

in which R¹, R², R³, R⁴, R⁵, R⁶, R⁷, R⁸, X and n are as defined supra,
then optionally converting said compound into solvates, salts and/or solvates of such salts using the corresponding (i) solvents and/or (ii) bases or acids.

In accordance with a second embodiment of the third aspect, the present invention covers methods of preparing compounds of general formula (I-b), which are compounds of general formula (I) in which R², R³, R⁴, R⁵, R⁶, R⁷, R⁸, X and n are as defined for the compound of general formula (I) as defined supra, and R¹ represents a carbamoyl group, said methods comprising the step of allowing a compound of general formula (I-a):

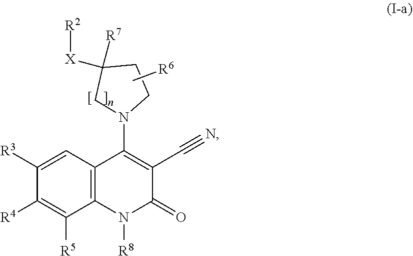

which is a compound of general formula (I) in which R², R³, R⁴, R⁵, R⁶, R⁷, R⁸, X and n are as defined for the compound of general formula (I) as defined supra, and R¹ represents a cyano group,
to react with palladium(II)acetate and acetaldoxime, thereby giving a compound of general formula (I-b)

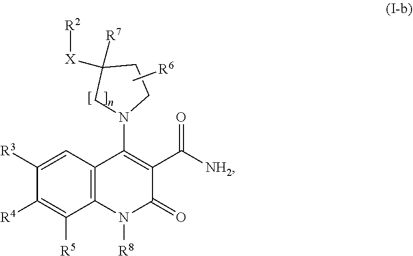

in which R², R³, R⁴, R⁵, R⁶, R⁷, R⁸, X and n are as defined supra, and R¹ represents a carbamoyl group,
then optionally converting said compound into solvates, salts and/or solvates of such salts using the corresponding (i) solvents and/or (ii) bases or acids.

In accordance with a third embodiment of the third aspect, the present invention covers methods of preparing compounds of general formula (I-c), which are compounds of general formula (I) in which R¹, R², R³, R⁴, R⁵, R⁶, R⁷, R⁸ and n are as defined for the compound of general formula (I) as defined supra, and X represents an O atom, said methods comprising the step of allowing an intermediate compound of general formula (IV)

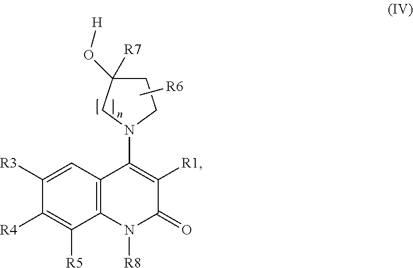

in which R¹, R³, R⁴, R⁵, R⁶, R⁷, R⁸ and n are as defined for the compound of general formula (I) as defined supra,
to react with a compound of general formula (V):

R²—OH (V),

in which R² is as defined for the compound of general formula (I) as defined supra, in the presence of triphenylphosphine and diisopropyl azodicarboxylate, thereby giving a compound of general formula (I-c)

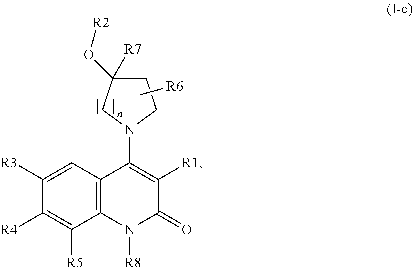

in which R¹, R², R³, R⁴, R⁵, R⁶, R⁷, R⁸ and n are as defined supra, and X represents an O atom,
then optionally converting said compound into solvates, salts and/or solvates of such salts using the corresponding (i) solvents and/or (ii) bases or acids.

In accordance with a fourth embodiment of the third aspect, the present invention covers methods of preparing compounds of general formula (I-e), which are compounds of general formula (I) in which R², R³, R⁴, R⁵, R⁶, R⁷, R⁸, X and n are as defined for the compound of general formula (I) as defined supra, and R¹ represents a —C(═O)NH₂, —C(═O)N(H)CH₃, —C(═O)N(H)C₂H₅ or —C(═O)N(CH₃)₂ group, said methods comprising the step of allowing a compound of general formula (I-d)

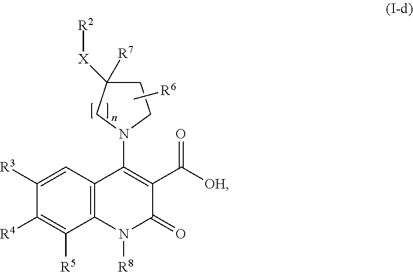

which is a compound of general formula (I) in which R², R³, R⁴, R⁵, R⁶, R⁷, R⁸, X and n are as defined for the compound of general formula (I) as defined supra, and R¹ represents a carboxyl group,
to react with a compound of general formula (VI)

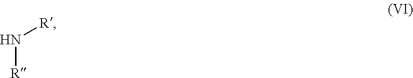

which compound is NH₃, H₂NCH₃, H₂NCH₂CH₃ or HN(CH₃)₂, or salts thereof,
thereby giving a compound of general formula (I-e)

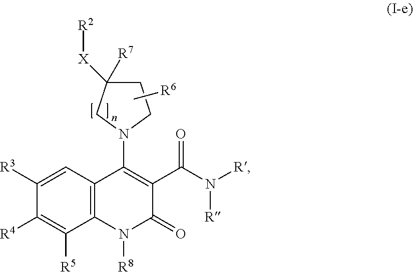

which is a compound of general formula (I) in which R², R³, R⁴, R⁵, R⁶, R⁷, R⁸, X and n are as defined supra, and R¹ represents a group —C(═O)NH₂, —C(═O)N(H)CH₃, —C(═O)N(H)C₂H₅ or —C(═O)N(CH₃)₂,
then optionally converting said compound into solvates, salts and/or solvates of such salts using the corresponding (i) solvents and/or (ii) bases or acids.

The present invention covers methods of preparing compounds of the present invention of general formula (I), said methods comprising the steps as described in the Experimental Section herein.

In accordance with a fourth aspect, the present invention covers the use of intermediate compounds for the preparation of a compound of general formula (I) as defined supra.

Particularly, the inventions covers the use of intermediate compounds of general formula (II)

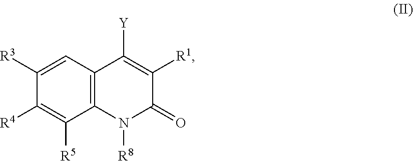

in which R¹, R³, R⁴, R⁵ and R⁸ are as defined for the compound of general formula (I) as defined supra, and Y has the meaning of chloro or bromo, for the preparation of a compound of general formula (I) as defined supra.

Particularly, the inventions covers the use of intermediate compounds of general formula (III):

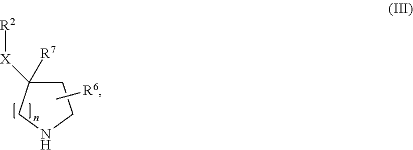

in which R², R⁶, R⁷, X and n are as defined for the compound of general formula (I) as defined supra, for the preparation of a compound of general formula (I) as defined supra.

Particularly, the inventions covers the use of intermediate compounds of general formula (IV):

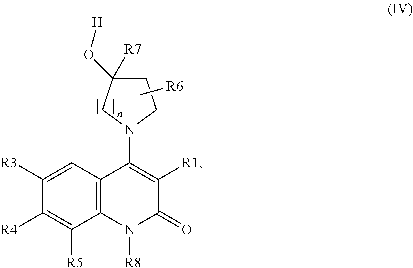

in which R¹, R³, R⁴, R⁵, R⁶, R⁷, R⁸ and n are as defined for the compound of general formula (I) as defined supra, for the preparation of a compound of general formula (I) as defined supra.

Particularly, the inventions covers the use of intermediate compounds of general formula (V):

R²—OH (V),

in which R² is as defined for the compound of general formula (I) as defined supra, for the preparation of a compound of general formula (I) as defined supra.

Particularly, the inventions covers the use of intermediate compounds of general formula (VI)

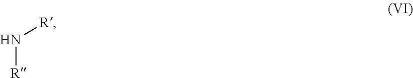

which compounds are NH₃, H₂NCH₃, H₂NCH₂CH₃ or HN(CH₃)₂, or salts thereof, for the preparation of a compound of general formula (I) as defined supra.

The present invention covers the use of intermediate compounds which are disclosed in the Example Section of this text, infra.

The present invention covers any sub-combination within any embodiment or aspect of the present invention of intermediate compounds of general formulae (II), (III), (IV), (V) and (VI), supra.

### DESCRIPTION OF THE FIGURES

FIG. 1: human DGKα M1 to S735 plus C-terminal Flag-Tag, DGKα_hu_1, as described under SEQ ID No. 1.

FIG. 2: human DGKα M1 to S735 plus N-terminal Avi-Tag and C-terminal Flag-Tag, DGKα_hu_1Avi, as described under SEQ ID No. 2.

FIG. 3: SIINFEKL amino acid sequence, as described under SEQ ID No. 3.

FIG. 4: GCCACC DNA sequence

FIG. 5: Flag-Tag sequence, as described under SEQ ID No. 4.

FIG. 6: OVA-30 peptide sequence, as described under SEQ ID No. 5.

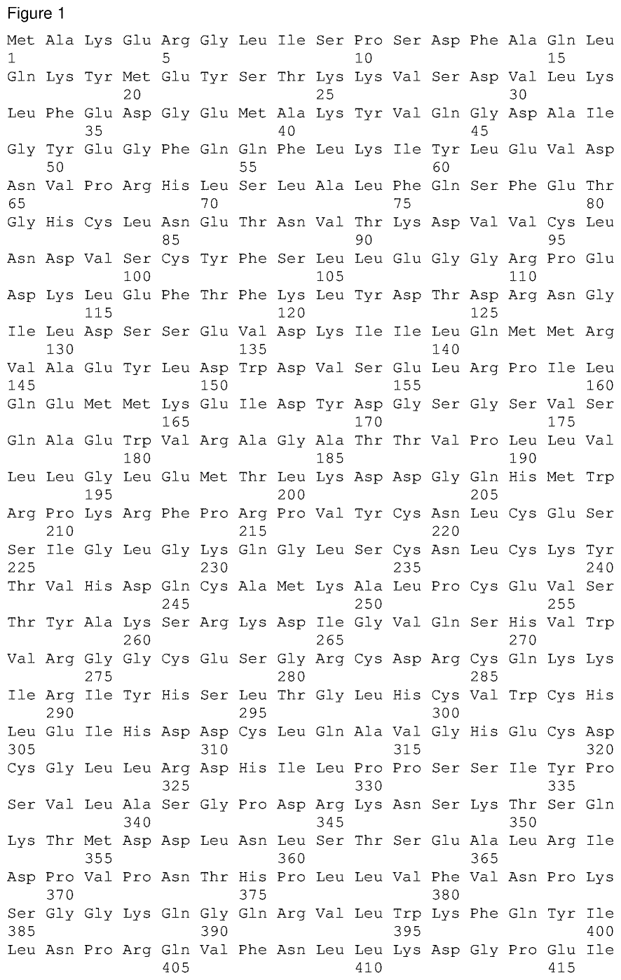

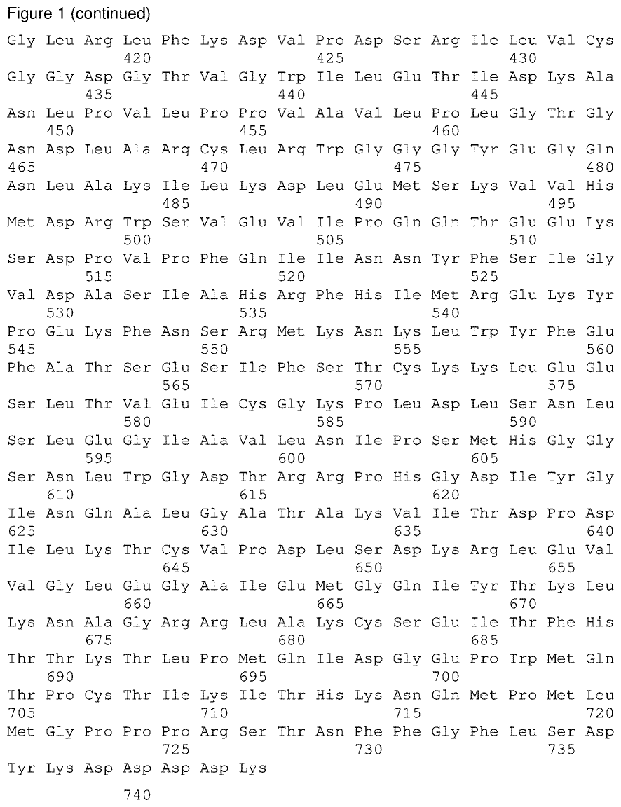

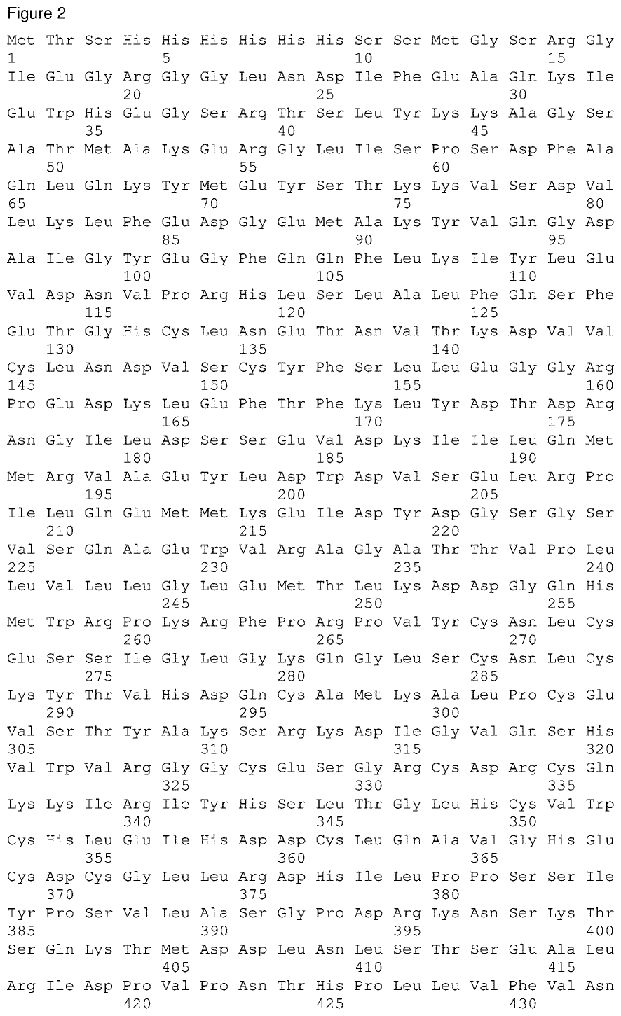

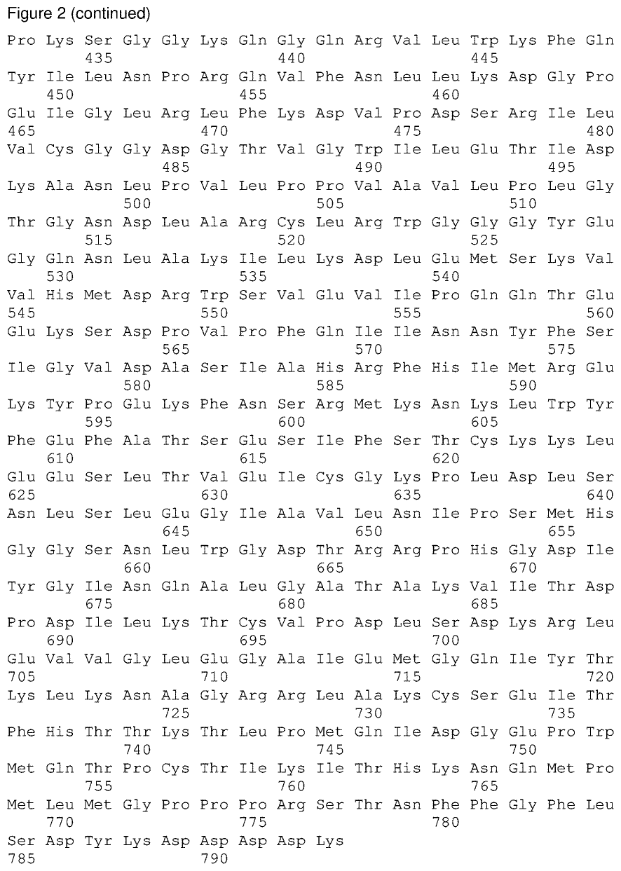

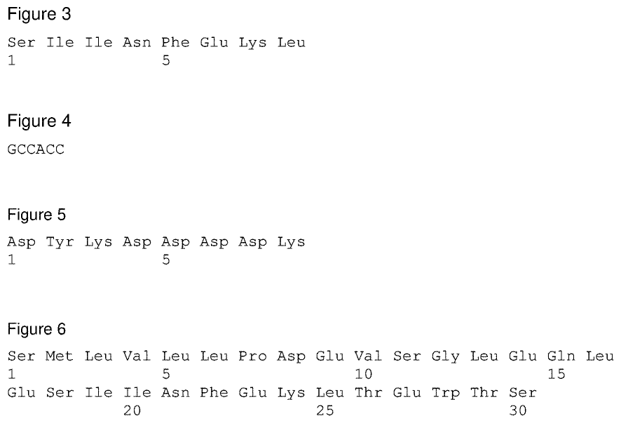

### EXPERIMENTAL SECTION

NMR peak forms are stated as they appear in the spectra, possible higher order effects have not been considered. The multiplicities are stated according to the signal form which appears in the spectrum, NMR-spectroscopic effects of a higher order were not taken into consideration. Multiplicity of the NMR signals: s=singlet, d=doublet, t=triplet, q=quartet, quin=quintet, spt=septed, br=broad signal, m=multiplet. NMR signals: shift in [ppm]. Combinations of multiplicity could be e.g. dd=doublet from doublet.

Chemical names were generated using the ACD/Name software from ACD/Labs. In some cases generally accepted names of commercially available reagents were used in place of ACD/Name generated names.

Table 1 lists the abbreviations used in this paragraph and in the examples section as far as they are not explained within the text body. Other abbreviations have their meanings customary per se to the skilled person.

TABLE 1
Abbreviations

| | |
|---|---|
| ACN | acetonitrile |
| AcOH | acetic acid |
| BOC | tert-butoxycarbonyl |
| CDCl3 | deuterochloroform |
| CFSE | carboxyfluorescein succinimidyl ester |
| DAD | diode array detector |
| DMF | N,N-dimethylformamide |
| DMSO-d6 | deuterated dimethyl sulphoxide |
| DMSO | dimethyl sulphoxide |
| ELSD | evaporative light scattering detector |
| ESIpos | electrospray ionization positive |
| Expl. | Example |
| h | hour/hours |
| HATU | (7-aza-1H-benzotriazol-1-yl)-1,1,3,3- |
| | tetramethyluronium hexafluorophosphate |
| HBTU | O-benzotriazole-N,N,N′,N′- |
| | tetramethyluronium hexafluorophosphate |
| HPLC | high-pressure liquid chromatography |
| LCMS | liquid chromatography coupled with mass |
| | spectrometry |
| LPS | lipopolysaccharide |
| mL | milliliter |
| min. | minute(s) |
| MTBE | methyl tert-butyl ether |
| PBMC | peripheral blood mononuclear cells |
| PyBOP | (benzotriazol-1-yl)oxytripyrrolidinophosphonium |
| | hexafluorophosphate |
| RP-HPLC | reverse-phase high-pressure liquid chromatography |
| rt | room temperature |
| Rt | retention time |
| sat. | saturated |
| T3P | 2,4,6-tripropyl-1,3,5,2,4,6-trioxatriphosphorinane |
| | 2,4,6-trioxide |
| THF | tetrahydrofurane |
| TFA | trifluoroacetic acid |
| TLC | thin layer chromatography |
| TNFα | tumour necrosis factor alpha |
| μM | micromolar |
| UPLC | Ultra high performance chromatography |

The various aspects of the invention described in this application are illustrated by the following examples which are not meant to limit the invention in any way.

The example testing experiments described herein serve to illustrate the present invention and the invention is not limited to the examples given.

### EXPERIMENTAL SECTION—GENERAL PART

All reagents, for which the synthesis is not described in the experimental part, are either commercially available, or are known compounds or may be formed from known compounds by known methods by a person skilled in the art.

The compounds and intermediates produced according to the methods of the invention may require purification. Purification of organic compounds is well known to the person skilled in the art and there may be several ways of purifying the same compound. In some cases, no purification may be necessary. In some cases, the compounds may be purified by crystallization. In some cases, impurities may be stirred out using a suitable solvent. In some cases, the compounds may be purified by chromatography, particularly flash column chromatography, using for example prepacked silica gel cartridges, e.g. Biotage SNAP cartidges KP-Sil® or KP-NH® in combination with a Biotage autopurifier system (SP4© or Isolera Four®) and eluents such as gradients of hexane/ethyl acetate or DCM/methanol. In some cases, the compounds may be purified by preparative HPLC using for example a Waters autopurifier system equipped with a diode array detector and/or on-line electrospray ionization mass spectrometer in combination with a suitable prepacked reverse phase column and eluents such as gradients of water and acetonitrile which may contain additives such as trifluoroacetic acid, formic acid or aqueous ammonia.

In some cases, purification methods as described above can provide those compounds of the present invention which possess a sufficiently basic or acidic functionality in the form of a salt, such as, in the case of a compound of the present invention which is sufficiently basic, a trifluoroacetate or formate salt for example, or, in the case of a compound of the present invention which is sufficiently acidic, an ammonium salt for example. A salt of this type can either be transformed into its free base or free acid form, respectively, by various methods known to the person skilled in the art, or be used as salts in subsequent biological assays. It is to be understood that the specific form (e.g. salt, free base etc.) of a compound of the present invention as isolated and as described herein is not necessarily the only form in which said compound can be applied to a biological assay in order to quantify the specific biological activity.

### Chromatographic Conditions

LC-MS (Method 1): Instrument: Waters Acquity UPLCMS SingleQuad; column: Acquity UPLC BEH C18 1.7 μm, 50×2.1 mm; eluent A: water+0.1 vol. % formic acid (99%), eluent B: acetonitrile; gradient: 0-1.6 min. 1-99% B, 1.6-2.0 min. 99% B; flow 0.8 ml/min; temperature: 60° C.; DAD scan: 210-400 nm.

LC-MS (Method 2): Instrument: Waters Acquity UPLCMS SingleQuad; column: Acquity UPLC BEH C18 1.7 μm, 50×2.1 mm; eluent A: water+0.2 vol. % aqueous ammonia (32%), eluent B: acetonitrile; gradient: 0-1.6 min. 1-99% B, 1.6-2.0 min. 99% B; flow 0.8 ml/min; temperature: 60° C.; DAD scan: 210-400 nm.

LC-MS (Method 3): Instrument: Agilent 1290 UPLCMS 6230 TOF; column: BEH C 18 1.7 μm, 50×2.1 mm; eluent A: water+0.05 vol. % formic acid (99%); eluent B: acetonitrile+0.05 vol. % formic acid (99%); gradient: 0-1.7 min. 2-90% B, 1.7-2.0 min. 90% B; flow 1.2 ml/min; temperature: 60° C.; DAD scan: 190-400 nm.

LC-MS (Method 4): Instrument: Agilent 1290 UPLCMS 6230 TOF; column: BEH C 18 1.7 μm, 50×2.1 mm; eluent A: water+0.05 vol. % formic acid (99%); eluent B: acetonitrile+0.05 vol. % formic acid (99%); gradient: 0-1.7 min. 2-90% B, 1.7-2.0 min. 90% B; flow 1.2 ml/min; temperature: 60° C.; DAD scan: 190-400 nm.

### EXPERIMENTAL SECTION—INTERMEDIATES

### Intermediate 1

### 4-(4-hydroxypiperidin-1-yl)-1-methyl-2-oxo-1,2-dihydroquinoline-3-carbonitrile

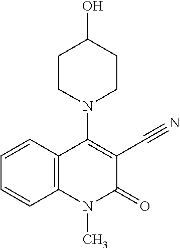 [SMILES: Cn1c(=O)c(C#N)c(N2CCC(O)CC2)c2ccccc21]

A suspension of 800 mg 4-chloro-1-methyl-2-oxo-1,2-dihydroquinoline-3-carbonitrile (3.66 mmol, CAS 150617-68-8, synthesis described in WO2012009649, example 1—compound Ill), 444 mg piperidin-4-ol (4.39 mmol, CAS 5382-16-1) and 1.9 mL N,N-diisopropylethylamine (11 mmol) in 18 mL 2-propanol was stirred for 2 h at 90° C. The mixture was cooled down to rt, water was added, the precipitate was collected by filtration, washed with ethanol and dried in vacuum. 820 mg of the title compound were obtained (75% yield, 95% purity).

¹H NMR (400 MHz, DMSO-d₆) δ ppm 1.66 (m, 2H), 1.96 (m, 2H), 3.41 (m, 2H), 3.56 (s, 3H), 3.72 (m, 2H), 3.84 (m, 1H), 4.89 (d, 1H), 7.34 (m, 1H), 7.56 (m, 1H), 7.70 (m, 1H), 7.82 (m, 1H).

LC-MS (Method 1): Rₜ=0.79 min; MS (ESIpos): m/z=284 [M+H]⁺

### Intermediate 2

### tert-butyl 4-[(methanesulfonyl)oxy]piperidine-1-carboxylate

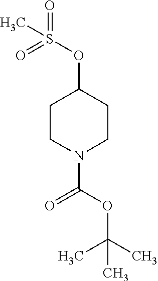 [SMILES: CC(C)(C)OC(=O)N1CCC(OS(C)(=O)=O)CC1]

To 10.0 g tert-butyl 4-hydroxypiperidine-1-carboxylate (49.7 mmol, CAS 109384-19-2) and 15 mL triethylamine (110 mmol) in 150 mL THF at 0° C. was added carefully 4.2 mL methanesulfonyl chloride (55 mmol) and the mixture was stirred 5 h at 0° C. The mixture was poured into an aqueous solution of bicarbonate and extracted with ethyl acetate (3×). The combined organic phases were washed with water, dried and concentrated under reduced pressure to give 13.8 g of the title compound (95% purity, 94% yield).

¹H NMR (400 MHz, DMSO-d₆) δ ppm 1.40 (s, 9H), 1.61 (m, 2H), 1.90 (m, 2H), 3.17 (m, 5H), 3.61 (m, 2H), 4.82 (m, 1H).

### Intermediate 3

### tert-butyl 4-[4-(1H-1,2,4-triazol-1-yl)phenoxy]piperidine-1-carboxylate

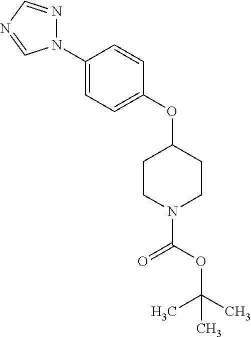 [SMILES: CC(C)(C)OC(=O)N1CCC(Oc2ccc(-n3cncn3)cc2)CC1]

A mixture of 500 mg tert-butyl 4-[(methanesulfonyl)oxy]piperidine-1-carboxylate (1.79 mmol, intermediate 2), 346 mg 4-(1H-1,2,4-triazol-1-yl)phenol (2.15 mmol, CAS 68337-15-5) and 1.17 g cesium carbonate (3.58 mmol) in 10 mL DMF was stirred for 6 h at 90° C. The mixture was poured into an aqueous solution of bicarbonate carbonate and extracted with ethyl acetate (3×). The combined organic phases were washed with water, dried and concentrated under reduced pressure. The residue was purified by flash chromatography (silica, dichloromethane/methanol gradient 0-3%) to give 250 mg of the title compound (98% purity, 40% yield).

¹H NMR (400 MHz, DMSO-d₆) δ ppm 1.40 (s, 9H), 1.53 (dtd, 2H), 1.92 (m, 2H), 3.18 (m, 2H), 3.67 (m, 2H), 4.63 (m, 1H), 7.15 (m, 2H), 7.75 (m, 2H), 8.18 (s, 1H), 9.16 (s, 1H).

LC-MS (Method 2): Rₜ=1.16 min; MS (ESIpos): m/z=245.5 [M+H]⁺

### Intermediate 4

### 4-[4-(1H-1,2,4-triazol-1-yl)phenoxy]piperidine

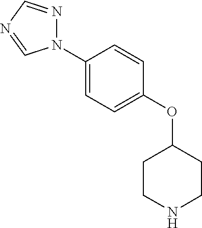 [SMILES: c1ncn(-c2ccc(OC3CCNCC3)cc2)n1]

To a solution of 250 mg tert-butyl 4-[4-(1H-1,2,4-triazol-1-yl)phenoxy]piperidine-1-carboxylate (726 μmol, intermediate 3) in 10 mL dichloromethane was added 1.1 mL trifluoroacetic acid (15 mmol) and the mixture was stirred for 2 h at rt. The mixture was concentrated under reduced pressure and the residue was diluted with toluene. The solvent was evaporated to give 200 mg TFA salt of the title compound (98% purity, 111% yield).

LC-MS (Method 2): Rₜ=0.74 min; MS (ESIpos): m/z=245.2 [M+H]⁺

### Intermediate 5

### tert-butyl 4-[4-(2-oxopyrrolidin-1-yl)phenoxy]piperidine-1-carboxylate

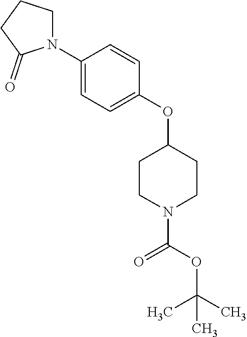 [SMILES: CC(C)(C)OC(=O)N1CCC(Oc2ccc(N3CCCC3=O)cc2)CC1]

A mixture of 500 mg tert-butyl 4-[(methanesulfonyl)oxy]piperidine-1-carboxylate (1-79 mmol, intermediate 2), 381 mg 1-(4-hydroxyphenyl)pyrrolidin-2-one (2.15 mmol, CAS 7517-07-9) and 1.17 g cesium carbonate (3.58 mmol) in 10 mL DMF was stirred for 5 h at 90° C. The mixture was poured into an aqueous solution of bicarbonate and extracted with ethyl acetate (2×). The combined organic phases were washed with brine, dried and concentrated under reduced pressure. The residue was purified by flash chromatography (silica, dichloromethane/methanol gradient 0-3%) to give 310 mg of the title compound (98% purity, 46% yield). 1H NMR (DMSO-d6, 400 MHz): δ=7.49-7.57 (m, 2H), 6.91-7.03 (m, 2H), 4.43-4.59 (m, 1H), 3.73-3.82 (m, 2H), 3.55-3.69 (m, 2H), 3.09-3.23 (m, 2H), 2.41-2.48 (m, 2H), 1.95-2.12 (m, 2H), 1.81-1.93 (m, 2H), 1.44-1.54 (m, 2H), 1.40 ppm (s, 9H).

LC-MS (Method 2): Rₜ=1.19 min; MS (ESIpos): m/z=362 [M+H]⁺

### Intermediate 6

### 1-{4-[(piperidin-4-yl)oxy]phenyl}pyrrolidin-2-one

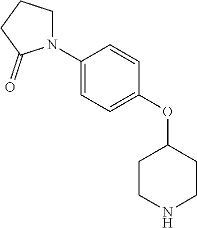 [SMILES: O=C1CCCN1c1ccc(OC2CCNCC2)cc1]

To a solution of 310 mg tert-butyl 4-[4-(2-oxopyrrolidin-1-yl)phenoxy]piperidine-1-carboxylate (860 μmol, intermediate 5) in 10 mL dichloromethane was added 1.3 mL trifluoroacetic acid (17 mmol) and the mixture was stirred for 2 h at rt. The mixture was concentrated under reduced pressure and the residue was diluted with toluene. The solvent was evaporated to give 300 mg TFA salt of the title compound (98% purity, 120% yield).

LC-MS (Method 2): Rₜ=0.79 min; MS (ESIpos): m/z=261.2 [M+H]⁺

### Intermediate 7

### tert-butyl 4-[4-(morpholin-4-yl)phenoxy]piperidine-1-carboxylate

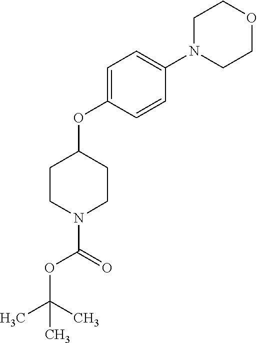 [SMILES: CC(C)(C)OC(=O)N1CCC(Oc2ccc(N3CCOCC3)cc2)CC1]

A mixture of 750 mg tert-butyl 4-bromopiperidine-1-carboxylate (2.84 mmol, CAS 180695-79-8), 509 mg 4-(morpholin-4-yl)phenol (2.84 mmol, CAS 6291-23-2) and 1.85 g cesium carbonate (5.68 mmol) in 20 mL DMF was stirred for 6 h at 90° C. and 5 h at 100° C. The mixture was stirred in water and ethyl acetate. The organic phase was washed with water and brine, dried and concentrated under reduced pressure. The residue was purified by flash chromatography (silica, dichloromethane/methanol gradient 0-3%) to give 80 mg of the title compound (80% purity, 6% yield).

¹H NMR (DMSO-d₆) δ: 6.86 (s, 4H), 4.33-4.46 (m, 1H), 3.68-3.75 (m, 4H), 3.56-3.66 (m, 2H), 3.08-3.21 (m, 2H), 2.93-3.01 (m, 4H), 1.78-1.89 (m, 2H), 1.43-1.52 (m, 2H), 1.36-1.41 (m, 9H). LC-MS (Method 2): Rₜ=1.32 min; MS (ESIpos): m/z=363.3 [M+H]⁺

### Intermediate 8

### 4-{4-[(piperidin-4-yl)oxy]phenyl}morpholine

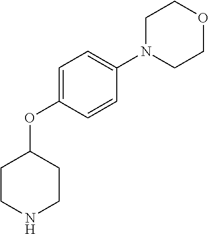 [SMILES: c1cc(N2CCOCC2)ccc1OC1CCNCC1]

To a solution of 12 mg tert-butyl 4-[4-(morpholin-4-yl)phenoxy]piperidine-1-carboxylate (33 μmol, intermediate 7) in 3 mL dichloromethane was added 51 μL trifluoroacetic acid (660 μmol) and the mixture was stirred for 2 h at rt. The mixture was concentrated under reduced pressure and the residue was diluted with toluene. The solvent was evaporated to give 10 mg of title compound (85% purity, 98% yield).

LC-MS (Method 2): Rₜ=0.88 min; MS (ESIpos): m/z=263.8 [M+H]⁺

### Intermediate 9

### tert-butyl 4-{[2-(trifluoromethyl)pyrimidin-5-yl]oxy}piperidine-1-carboxylate

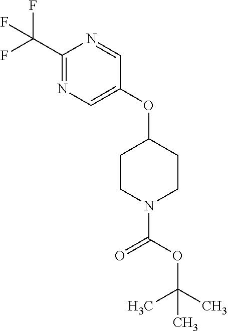 [SMILES: CC(C)(C)OC(=O)N1CCC(Oc2cnc(C(F)(F)F)nc2)CC1]

A mixture of 500 mg tert-butyl 4-[(methylsulfonyl)oxy]piperidine-1-carboxylate (1.79 mmol, intermediate 2), 352 mg 2-(trifluoromethyl)pyrimidin-5-ol (2.15 mmol, CAS 100991-09-1) and 1.17 g cesium carbonate (3.58 mmol) in 10 mL DMF was stirred for 5 h at 90° C. The mixture was poured into an aqueous solution of bicarbonate carbonate and extracted with ethyl acetate (2×). The combined organic phases were washed with brine, dried and concentrated under reduced pressure to give 470 mg of the title compound (80% purity, 60% yield).

¹H NMR (400 MHz, DMSO-d₆) δ ppm 1.40 (s, 9H); 1.53-1.65 (m, 2H); 1.94-2.02 (m, 2H); 3.14-3.26 (m, 2H); 3.62-3.73 (m, 2H); 4.87-4.97 (m, 1H); 8.81 (s, 2H).

LC-MS (Method 2): Rₜ=1.29 min; MS (ESIpos): m/z=292.4 [M+H]⁺

### Intermediate 10

### 5-[(piperidin-4-yl)oxy]-2-(trifluoromethyl)pyrimidine

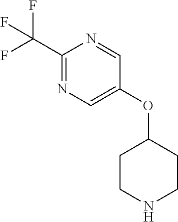 [SMILES: FC(F)(F)c1ncc(OC2CCNCC2)cn1]

To a solution of 470 mg tert-butyl 4-{[2-(trifluoromethyl)pyrimidin-5-yl]oxy}piperidine-1-carboxylate (1.35 mmol, intermediate 9) in 15 mL dichloromethane was added 2.1 mL trifluoroacetic acid (27 mmol) and the mixture was stirred for 2 h at rt. The mixture was concentrated under reduced pressure and the residue was diluted with toluene. The solvent was evaporated to give 400 mg of title compound (80% purity, 96% yield).

LC-MS (Method 2): Rₜ=0.89 min; MS (ESIpos): m/z=248.1 [M+H]⁺

### Intermediate 11

### tert-butyl 4-[4-(difluoromethoxy)phenoxy]piperidine-1-carboxylate

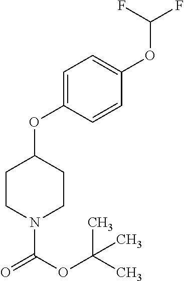 [SMILES: CC(C)(C)OC(=O)N1CCC(Oc2ccc(OC(F)F)cc2)CC1]

To 500 μL 4-(difluoromethoxy)phenol (3.9 mmol, CAS 87789-47-7) in 15 mL DMF at 0° C. was added 343 mg sodium hydride (60% in mineral oil, 8.58 mmol) and the mixture was stirred for 30 min. at 0° C. A solution of 1.49 g of tert-butyl 4-[(methanesulfonyl)oxy]piperidine-1-carboxylate (5.07 mmol, intermediate 2) in 15 mL DMF was added dropwise and the mixture was stirred for 65 h at 80° C. The mixture was cooled down to rt, water was added and concentrated under reduced pressure. The residue was purified by RP-HPLC (column: X-Bridge C18 5 μm 100×30 mm, mobile phase: water (0.2 vol. % ammonia 32%)/acetonitrile-gradient) to give 633 mg of the title compound (100% purity, 47% yield).

¹H NMR (DMSO-d₆) δ: 7.06-7.17 (m, 1H), 6.97-7.05 (m, 1H), 6.87-7.34 (m, 3H), 4.43-4.58 (m, 1H), 3.56-3.72 (m, 2H), 3.05-3.26 (m, 2H), 1.82-1.95 (m, 2H), 1.43-1.57 (m, 2H), 1.35-1.43 (m, 9H).

LC-MS (Method 2): Rₜ=1.42 min; MS (ESIpos): m/z=288 [M+H]⁺

### Intermediate 12

### 4-[4-(difluoromethoxy)phenoxy]piperidine, salt with hydrochloric acid

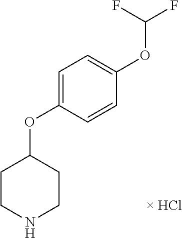 [SMILES: FC(F)Oc1ccc(OC2CCNCC2)cc1]

To a solution of 694 mg tert-butyl 4-[4-(difluoromethoxy)phenoxy]piperidine-1-carboxylate (2.02 mmol, intermediate 11) in 35 mL methanol was added 5.1 mL hydrogen chloride (4.0 M in dioxane, 20 mmol) and the mixture was stirred for 4 h at rt. The mixture was concentrated under reduced pressure and dried in vacuum to give 566 mg of the title compound (100% purity, 100% yield).

LC-MS (Method 2): Rₜ=1.02 min; MS (ESIpos): m/z=244.2 [M+H]⁺

### Intermediate 13

### tert-butyl 4-methyl-4-(4-nitrophenoxy)piperidine-1-carboxylate

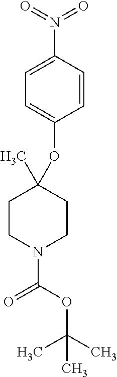 [SMILES: CC(C)(C)OC(=O)N1CCC(C)(Oc2ccc([N+](=O)[O-])cc2)CC1]

To a suspension of 92.9 mg sodium hydride (60% in mineral oil, 2.32 mmol) in 2.5 mL THF at 0° C. was added 250 mg tert-butyl 4-hydroxy-4-methylpiperidine-1-carboxylate (1.16 mmol, CAS 406235-30-1) solved in 5 mL THF and the mixture was stirred for 15 min. at 0° C. 46 mg 1-fluoro-4-nitrobenzene (1.74 mmol, CAS 350-46-9) solved in 2 mL THF was added dropwise and the mixture was stirred for 6 h at 70° C. After cooling to rt, water was added and the mixture was extracted with ethyl acetate (3×). The combined organic phases were washed with brine, dried and concentrated under reduced pressure. The residue was purified by flash chromatography (silica, hexane/ethyl acetate gradient 0-10%) to give 290 mg of the title compound (95% purity, 71% yield).

¹H NMR (DMSO-d₆) δ: 8.12-8.19 (m, 2H); 7.20-7.32 (m, 2H); 3.51-3.69 (m, 2H); 3.00-3.28 (m, 2H); 1.90-2.04 (m, 2H); 1.57-1.73 (m, 2H); 1.42-1.45 (m, 3H); 1.36-1.41 (m, 9H).

### Intermediate 14

### 4-methyl-4-(4-nitrophenoxy)piperidine, salt with trifluoroacetic acid

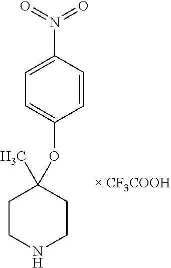 [SMILES: CC1(Oc2ccc([N+](=O)[O-])cc2)CCNCC1]

To a solution of 275 mg tert-butyl 4-methyl-4-(4-nitrophenoxy)piperidine-1-carboxylate (818 μmol, intermediate 13) in 5.3 mL dichloromethane was added 630 μL trifluoroacetic acid (8.2 mmol) and the mixture was stirred overnight at rt. The mixture was concentrated under reduced pressure and the residue was diluted with toluene. The solvent was evaporated to give 280 mg TFA salt of the title compound (95% purity, 138% yield).

¹H NMR (DMSO-d₆) δ: 8.41-8.61 (m, 1H), 8.16-8.24 (m, 2H), 7.26-7.34 (m, 2H), 3.02-3.26 (m, 4H), 2.12-2.22 (m, 2H), 1.81-1.94 (m, 2H), 1.41-1.48 (m, 3H).

LC-MS (Method 2): Rₜ=1.00 min; MS (ESIpos): m/z=237.2 [M+H]⁺

### Intermediate 15

### tert-butyl 4-[4-(methanesulfonyl)phenoxy]piperidine-1-carboxylate

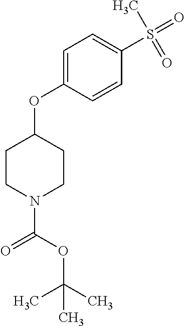 [SMILES: CC(C)(C)OC(=O)N1CCC(Oc2ccc(S(C)(=O)=O)cc2)CC1]

To a suspension of 100 mg tert-butyl 4-hydroxypiperidine-1-carboxylate (497 μmol, CAS 109384-19-2), 94.1 mg 4-(methanesulfonyl)phenol (547 μmol, CAS 14763-60-1) and 143 mg triphenylphosphine (547 μmol) was added 110 μL diisopropyl azodicarboxylate (550 μmol) at 0° C. The mixture was stirred overnight at rt. After that, water was added and the mixture was extracted with ethyl acetate (3×). The combined organic phases were filtered (using a waterresistant filter) and concentrated under reduced pressure. The residue was purified by flash chromatography (silica, hexane/ethyl acetate gradient 0-70%). The impure product was purified by flash chromatography again (silica, dichloromethane/methanol gradient 0-20%). The impure product was purified by RP-HPLC (column: Chromatorex 125×30 mm, 10 μm mobile phase: acetonitrile/water (0.2 vol. % ammonia 32%)-gradient) to give 76 mg of the title compound (99% purity, 43% yield).

¹H NMR (DMSO-d₆) δ: 7.75-7.92 (m, 2H), 7.15-7.26 (m, 2H), 4.66-4.81 (m, 1H), 3.60-3.72 (m, 2H), 3.09-3.25 (m, 5H), 1.87-1.97 (m, 2H), 1.48-1.62 (m, 2H), 1.36-1.45 (m, 9H).

LC-MS (Method 1): Rₜ=1.14 min; MS (ESIpos): m/z=300.1 [M+H]⁺

### Intermediate 16

### 4-[4-(methanesulfonyl)phenoxy]piperidine

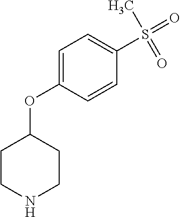 [SMILES: CS(=O)(=O)c1ccc(OC2CCNCC2)cc1]

To a solution of 70 mg tert-butyl 4-[4-(methanesulfonyl)phenoxy]piperidine-1-carboxylate (197 μmol, intermediate 15) in 2 mL dichloromethane was added 380 μL trifluoroacetic acid (4.9 mmol) and the mixture was stirred for 1.5 h at rt. To the mixture was added a solution of sat. sodium bicarbonate and extracted with dichloromethane (3×). The combined organic phases were filtered (using a waterresistant filter) and concentrated under reduced pressure to give 20 mg of the title compound (95% purity, 38% yield).

¹H NMR (DMSO-d₆) δ: 7.74-7.83 (m, 2H), 7.11-7.22 (m, 2H), 4.50-4.62 (m, 1H), 3.10-3.19 (m, 3H), 2.94 (dt, J=12.8, 4.1 Hz, 2H), 2.54-2.63 (m, 2H), 1.88-1.99 (m, 2H), 1.40-1.53 (in, 2H).

TABLE 2
Compounds in table 2 were prepared in analogy to intermediate 15.

| Inter- | | | | |
|---|---|---|---|---|
| mediate | Structure | IUPAC-Name | Starting Materials | Analytics |
| 17 | 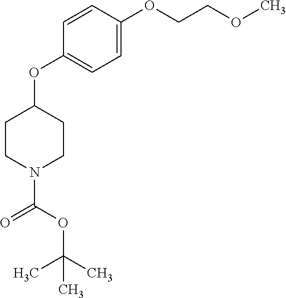 [SMILES: COCCOc1ccc(OC2CCN(C(=O)OC(C)(C)C)CC2)cc1] | tert-butyl 4-[4- (2- methoxyethoxy) phenoxy] piperidine-1- carboxylate | tert-butyl 4- hydroxypiperidine-1- carboxylate (CAS 109384-19-2) and 4-(2- methoxyethoxy)phenol (CAS 51980-60-0) | ¹H NMR (DMSO-d₆) δ: 6.81-6.96 (m, 4H), 4.31-4.47 (m, 1H), 3.98-4.03 (m, 2H), 3.60-3.68 (m, 4H), 3.27-3.31 (m, 3H), 3.06-3.19 (m, 2H), 1.79-1.91 (m, 2H), 1.42-1.51 (m, 2H), 1.36-1.42 (s, 9H). |
| 18 | 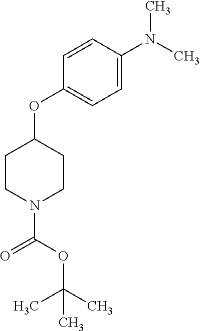 [SMILES: CN(C)c1ccc(OC2CCN(C(=O)OC(C)(C)C)CC2)cc1] | tert-butyl 4-[4- (dimethylamino) phenoxy] piperidine-1- carboxylate | tert-butyl 4- hydroxypiperidine-1- carboxylate (CAS 109384-19-2) and 4- (dimethylamino) phenol (CAS 619-60-3) | ¹H NMR (DMSO-d₆) δ: 6.80-6.89 (m, 2H), 6.64-6.72 (m, 2H), 4.24-4.39 (m, 1H), 3.62 (ddd, 2H), 3.04-3.22 (m, 2H), 2.76-2.82 (m, 6H), 1.77-1.89 (m, 2H), 1.46 (dtd, 2H), 1.40 (s, 9H). |
| 19 | 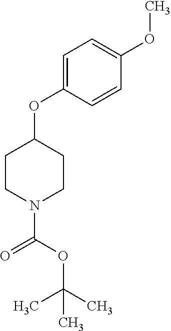 [SMILES: COc1ccc(OC2CCN(C(=O)OC(C)(C)C)CC2)cc1] | tert-butyl 4-(4- methoxyphenoxy) piperidine-1- carboxylate | tert-butyl 4- hydroxypiperidine-1- carboxylate (CAS 109384-19-2) and 4- methoxyphenol (CAS 150-76-5) | ¹H NMR (DMSO-d₆) δ: 6.80-6.94 (m, 4H), 4.39 (tt, J = 7.9, 3.7 Hz, 1H), 3.58- 3.72 (m, 5H), 3.04- 3.28 (m, 2H), 1.79- 1.93 (m, 2H), 1.43- 1.52 (m, 2H), 1.38- 1.42 (s, 9H). |
| 20 | 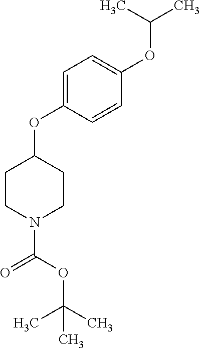 [SMILES: CC(C)Oc1ccc(OC2CCN(C(=O)OC(C)(C)C)CC2)cc1] | tert-butyl 4-{4- [(propan-2- yl)oxy]phenoxy} piperidine-1- carboxylate | tert-butyl 4- hydroxypiperidine-1- carboxylate (CAS 109384-19-2) and 4- isopropoxyphenol (CAS 7495-77-4) | ¹H-NMR (400 MHz, DMSO-d₆): δ [ppm] = 1.22 (d, 6H), 1.40 (s, 9H), 1.47 (dtd, 2H), 1.79- 1.91 (m, 2H), 3.14 (br t, 2H), 3.58- 3.71 (m, 2H), 4.38 (tt, 1H), 4.42-4.52 (m, 1H), 6.78-6.84 (m, 2H), 6.85-6.90 (m, 2H). |

TABLE 3
synthesis in analogy to intermediate 16.

| Inter- | | | Starting | |
|---|---|---|---|---|
| mediate | Structure | IUPAC-Name | Materials | Analytics |
| 21 | 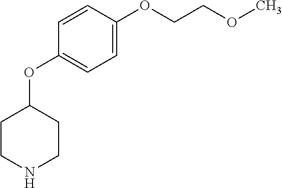 [SMILES: COCCOc1ccc(OC2CCNCC2)cc1] | 4-[4-(2- methoxyethoxy) phenoxy] piperidine | tert-butyl 4-[4-(2- methoxyethoxy) phenoxy]piperidine-1- carboxylate (intermediate 17) | ¹H NMR (DMSO-d₆) δ: 6.81-6.94 (m, 4H), 4.30-4.39 (m, 1H), 3.97-4.05 (m, 2H), 3.58-3.65 (m, 2H), 3.00-3.09 (m, 2H), 2.70-2.81 (m, 2H), 1.85-2.00 (m, 2H), 1.46-1.61 (m, 2H), 1.16-1.19 (m, 3H). |
| 22 | 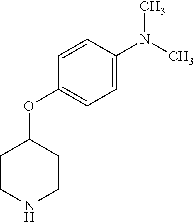 [SMILES: CN(C)c1ccc(OC2CCNCC2)cc1] | N,N-dimethyl-4- [(piperidin-4- yl)oxy]aniline | tert-butyl 4-[4- (dimethylamino) phenoxy]piperidine-1- carboxylate (intermediate 18) | ¹H NMR (DMSO-d₆) δ: 6.77-6.89 (m, 2H), 6.65-6.71 (m, 2H), 4.69-4.86 (m, 2H), 4.19-4.34 (m, 1H), 2.99-3.12 (m, 2H), 2.78-2.83 (m, 6H), 2.70-2.78 (m, 2H), 1.85-1.96 (m, 2H), 1.49-1.61 (m, 2H). |
| 23 | 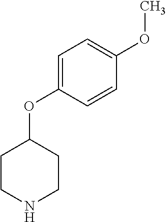 | 4-(4- methoxyphenoxy) piperidine | tert-butyl 4-(4- methoxyphenoxy) piperidine-1- carboxylate (intermediate 19) | ¹H NMR (DMSO-d₆) δ: 6.80-6.97 (m, 4H), 4.30-4.44 (m, 1H), 3.69 (s, 3H), 3.04- 3.14 (m, 2H), 2.76- 2.88 (m, 2H), 1.89- 2.00 (m, 2H), 1.53- 1.67 (m, 2H). |
| 24 | 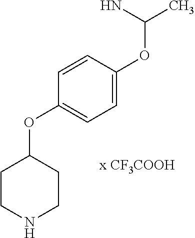 [SMILES: CC(N)Oc1ccc(OC2CCNCC2)cc1] | 4-{4-[(propan-2- yl)oxy]phenoxy} piperidine, salt with trifluoroacetic acid | tert-butyl 4-(4- isopropoxyphenoxy) piperidine-1- carboxylate (intermediate 20) | ¹H NMR (400 MHz, DMSO-d₆) δ ppm 8.39-8.70 (m, 2 H), 6.88-6.94 (m, 2 H), 6.81-6.87 (m, 2 H), 4.42-4.54 (m, 2 H), 3.23 (br dd, 2 H), 2.99-3.14 (m, 2 H), 2.03 (d quin, 2 H), 1.77 (ddt, 2 H), 1.22 (d, 6 H). LC-MS (Method 2): Rₜ = 1.11 min; MS (ESIpos): m/z = 236.2 [M + H]⁺ |

### Intermediate 25

### 7-bromo-1-methyl-2H-3,1-benzoxazine-2,4(1H)-dione

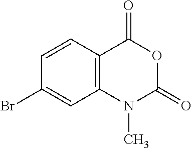

To a solution of 50 g 7-bromo-2H-3,1-benzoxazine-2,4(1H)-dione (207 mmol, CAS 76561-16-5) and 72 mL N,N-diisopropylethylamine (413 mmol) in 400 mL dimethylacetamide was added 39 mL iodomethane (620 mmol) at rt and the mixture was stirred overnight. The reaction was cooled to 0° C. and 200 mL water was slowly added. The solid that precipitated from this procedure was collected by filtration, washed with water and dried in an oven at 50° C. 48.1 g of the title compound was obtained (91% yield).

¹H-NMR (400 MHz, DMSO-d6) δ[ppm]: 3.46 (s, 3H); 7.52 (dd, 1H); 7.70 (d, 1H); 7.90 (d, 1H).

### Intermediate 26

### 7-bromo-4-hydroxy-1-methyl-2-oxo-1,2-dihydroquinoline-3-carbonitrile

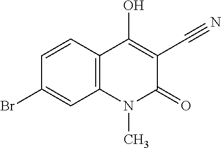 [SMILES: Cn1c(=O)c(C#N)c(O)c2ccc(Br)cc21]

A solution of 40 g 7-bromo-1-methyl-2H-3,1-benzoxazine-2,4(1H)-dione (156 mmol, intermediate 25) in 320 mL THF was slowly treated with 170 mL triethylamine (1.2 mol) followed by the addition of 25 mL ethyl cyanoacetate (234 mmol) at rt. The reaction was heated at 60° C. and stirred at that temperature over night. Further 25 mL ethyl cyanoacetate (234 mmol) were added and the reaction was stirred at 70° C. for further 5 h. After cooling to rt, water was added and THF was evaporated in vacuum. The mixture was acidified to pH=1 by addition of hydrochloric acid (2 M) and extracted with ethyl acetate 3 times. The combined organic layers were evaporated in vacuum and the residue was stirred first with hexane, decanted and then stirred with a small amount ethyl acetate/hexane. The residue was filtered and 46 g of the title material was obtained in two crops (106% yield).

¹H-NMR (400 MHz, DMSO-d6) δ[ppm]: 3.51 (s, 3H); 6.67 (bs, 1H); 7.46 (dd, 1H); 7.71 (d, 1H); 7.96 (d, 1H).

### Intermediate 27

### 7-bromo-4-chloro-1-methyl-2-oxo-1,2-dihydroquinoline-3-carbonitrile

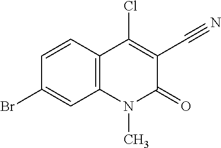 [SMILES: Cn1c(=O)c(C#N)c(Cl)c2ccc(Br)cc21]

A mixture of 16 g 7-bromo-4-hydroxy-1-methyl-2-oxo-1,2-dihydroquinoline-3-carbonitrile (57 mmol, intermediate 26) and 100 mL phosphoric trichloride (1.05 mol) was stirred at 90° C. overnight. After cooling to rt, hexane was added and the reaction was filtered. The solid was washed with sat. sodium bicarbonate solution and water. The obtained residue was dried in an oven at 50° C. overnight to give 13.2 g of the title compound (77% yield).

¹H-NMR (400 MHz, DMSO-d6) δ[ppm]: 3.64 (s, 3H); 7.66 (dd, 1H); 7.94-7.98 (m, 2H).

### Intermediate 28

### 7-methoxy-1-methyl-2H-3,1-benzoxazine-2,4(1H)-dione

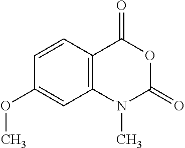

To a solution of 420 g potassium carbonate in 300 mL DMF was added 235 g 7-methoxy-2H-3,1-benzoxazine-2,4(1H)-dione (1.22 mol, CAS 128076-63-1) and 259 g iodomethane (1.82 mol, 113 ml). The mixture was stirred at 20° C. for 12 h. The reaction was monitored by LC-MS until complete consumption of 7-methoxy-2H-3,1-benzoxazine-2,4(1H)-dione. The reaction mixture was added to ice water (2000 ml), stirred for 0.5 h and filtered and concentrated under vacuum to give 218 g of the title compound (87% yield) as yellow solid.

¹H-NMR (400 MHz, DMSO-d6) δ[ppm]: 7.92 (d, 3H); 6.91 (dd, 1H); 6.83 (d, 1H); 3.93 (s, 3H); 3.45 (s, 3H).

### Intermediate 29

### 4-hydroxy-7-methoxy-1-methyl-2-oxo-1,2-dihydroquinoline-3-carbonitrile

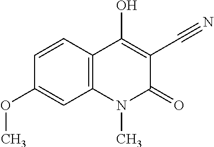 [SMILES: COc1ccc2c(O)c(C#N)c(=O)n(C)c2c1]

To a solution of 218 g 7-methoxy-1-methyl-2H-3,1-benzoxazine-2,4(1H)-dione (intermediate 28) and 159 g ethyl cyanoacetate in 2 L DMF was added 264 g potassium tert-butoxide. The mixture was stirred at 120° C. for 12 h. The reaction mixture was poured into 1000 mL water, extracted with ethyl acetate, the aqueous phase was adjusted pH=2˜3 with 6 M hydrochloric acid (400 ml) and stirred for 1 h at 0° C., then filtered and dried over sodium sulfate and evaporated in vacuum to give 130 g of the title compound (48% yield) as a yellow solid.

¹H-NMR (400 MHz, DMSO-d6) δ[ppm]: 8.03 (d, 1H); 6.96-6.92 (m, 2H); 3.93 (s, 3H); 3.54 (s, 3H).

### Intermediate 30

### 7-bromo-4-chloro-1-methyl-2-oxo-1,2-dihydroquinoline-3-carbonitrile

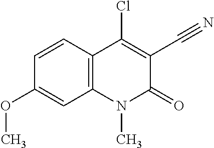 [SMILES: COc1ccc2c(Cl)c(C#N)c(=O)n(C)c2c1]

A solution of 50 g 4-hydroxy-7-methoxy-1-methyl-2-oxo-1,2-dihydroquinoline-3-carbonitrile (intermediate 29) in 430 mL phosphoric trichloride was stirred at 110° C. for 12 h. The solvent was removed in vacuum and to the residue was added 500 mL ice water and stirred for 12 h. The reaction mixture was filtered and concentrated under vacuum to give 47.5 g of the title compound (88% yield) as a brown solid.

¹H-NMR (400 MHz, DMSO-d6) δ[ppm]: 7.97 (d, 1H); 7.11 (dd, 1H); 7.05 (d, 1H); 3.99 (s, 3H); 3.64 (s, 3H).

### Intermediate 31

### 4-chloro-7-hydroxy-1-methyl-2-oxo-1,2-dihydroquinoline-3-carbonitrile

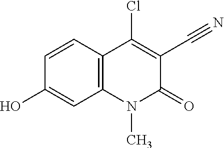 [SMILES: Cn1c(=O)c(C#N)c(Cl)c2ccc(O)cc21]

To a solution of 15 g 7-bromo-4-chloro-1-methyl-2-oxo-1,2-dihydroquinoline-3-carbonitrile (intermediate 30) in 350 mL dichloromethane at −40° C. was added 117 g boron tribromide (45.0 ml). Then the mixture was stirred at 20° C. for 12 h. The conversion was checked by LC-MS to indicate ˜10% of left starting material. The residue was poured into 6 L ice water and filtered.

To the reaction mixture was added 1.5 L sat. aqueous sodium bicarbonate solution and the mixture was stirred for 2 h, then the mixture was filtered and dried over sodium sulfate. The solvent was removed in vacuum and to the resulting residue was added 500 mL DMSO. The mixture was stirred for 3 h, then filtered and washed with water (100 ml, 3X), the product was dried in vacuum to give 3.34 g of the title compound (24% yield) as a light brown solid.

¹H-NMR (400 MHz, DMSO-d6) δ[ppm]: 11.28 (brs, 1H); 7.89 (d, 1H); 6.94 (dd, 1H); 6.85 (s, 1H); 3.55 (s, 3H).

### Intermediate 32

### 7-fluoro-1-methyl-2H-3,1-benzoxazine-2,4(1H)-dione

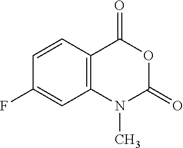

To a solution of 5 g 7-fluoro-2H-3,1-benzoxazine-2,4(1H)-dione (26.2 mmol, CAS 321-50-6) and 9.1 mL N,N-diisopropylethylamine (52 mmol) in 400 mL DMF was added 4.9 mL iodomethane (79 mmol) and was stirred overnight at rt. To the mixture was added ice water. The solid that precipitated from this procedure was collected by filtration, washed with water and hexane and dried in vacuum to give 3.75 g of the title compound (95% purity, 70% yield).

¹H NMR (DMSO-d₆) δ: 8.08 (dd, 1H), 7.40 (dd, 1H), 7.19 (td, 1H), 3.44 (s, 3H).

LC-MS (Method 1): Rₜ=0.79 min; MS (ESIpos): m/z=196.1 [M+H]⁺

### Intermediate 33

### 7-fluoro-4-hydroxy-1-methyl-2-oxo-1,2-dihydroquinoline-3-carbonitrile

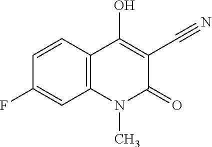 [SMILES: Cn1c(=O)c(C#N)c(O)c2ccc(F)cc21]

A suspension of 3.7 g 7-fluoro-1-methyl-2H-3,1-benzoxazine-2,4(1H)-dione (18 mmol, intermediate 32) in 40 mL 2-methyltetrahydrofuran was slowly treated with 20 mL triethylamine (144 mmol) followed by the addition of 7.7 mL ethyl cyanoacetate (72 mmol) and was refluxed for 73 h. After cooling to rt, the solvent was evaporated in vacuum, water and ethyl acetate was added and the mixture was acidified to pH=1 by addition of hydrochloric acid (2 M). The solid that precipitated from this procedure was collected by filtration, washed with water and hexane and dried in vacuum to give 3.09 g of the title compound (100% purity, 73% yield).

¹H NMR (DMSO-d₆) δ: 8.11 (dd, 1H), 7.37 (dd, 1H), 7.16 (td, 1H), 3.49 (s, 3H).

LC-MS (Method 1): Rₜ=0.68 min; MS (ESIneg): m/z=217.1 [M−H]⁻

### Intermediate 34

### 4-chloro-7-fluoro-1-methyl-2-oxo-1,2-dihydroquinoline-3-carbonitrile

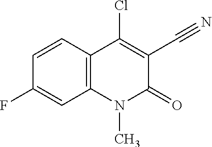 [SMILES: Cn1c(=O)c(C#N)c(Cl)c2ccc(F)cc21]

A mixture of 3 g 7-fluoro-4-hydroxy-1-methyl-2-oxo-1,2-dihydroquinoline-3-carbonitrile (13.7 mmol, intermediate 33) and 20 mL phosphoric trichloride (208 mmol) was stirred for 67 h at 70° C. and 4 h at 90° C. After cooling to rt, dichloro methane and ice water were added and the mixture was extracted with dichloromethane (3×). The combined organic phases were washed with brine, filtered (using a waterresistant filter) and concentrated under reduced pressure to give 2.46 g of the title compound (96% purity, 73% yield).

¹H NMR (DMSO-d₆) δ: 8.15 (dd, 1H), 7.67 (dd, 1H), 7.35-7.42 (m, 1H), 3.63 (s, 3H).

LC-MS (Method 1): Rₜ=1.00 min; MS (ESIneg): m/z=235.1 [M−H]⁻

### Intermediate 35

### 7-chloro-1-methyl-2H-3,1-benzoxazine-2,4(1H)-dione

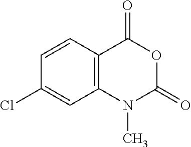

To a solution of 2 g 7-chloro-2H-3,1-benzoxazine-2,4(1H)-dione (9.82 mmol, CAS 40928-13-0) and 3.4 mL N,N-diisopropylethylamine (20 mmol) in 15 mL DMF was added 1.9 mL iodomethane (29 mmol) and was stirred overnight at rt. To the mixture was added ice water. The solid that precipitated from this procedure was collected by filtration, washed with water and hexane and dried in vacuum to give 2.50 g of the title compound (98% purity, 118% yield).

¹H NMR (DMSO-d₆) δ: 8.00 (d, 1H), 7.59 (d, 1H), 7.39 (dd, 1H), 3.46 (s, 3H).

LC-MS (Method 1): Rₜ=0.90 min; MS (ESIpos): m/z=212.1 [M+H]⁺

### Intermediate 36

### 7-chloro-4-hydroxy-1-methyl-2-oxo-1,2-dihydroquinoline-3-carbonitrile

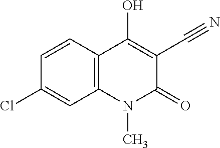 [SMILES: Cn1c(=O)c(C#N)c(O)c2ccc(Cl)cc21]

A suspension of 2.5 g 7-chloro-1-methyl-2H-3,1-benzoxazine-2,4(1H)-dione (11.6 mmol, intermediate 35) in 25 mL 2-methyltetrahydrofuran was slowly treated with 13 mL triethylamine (93 mmol) followed by the addition of 4.9 mL ethyl cyanoacetate (46 mmol) and was refluxed for 65 h. After cooling to rt, the solvent was evaporated in vacuum, water/ethyl acetate (1:1) was added and the mixture was acidified to pH=1 by addition of hydrochloric acid (2 M). The solid that precipitated from this procedure was collected by filtration, washed with water, ethyl acetate and hexane and dried in vacuum to give 2.55 g of the title compound (100% purity, 94% yield).

¹H NMR (DMSO-d₆) δ: 8.05 (d, 1H), 7.59 (d, 1H), 7.34 (dd, 1H), 3.52 (s, 3H).

LC-MS (Method 2): Rₜ=0.57 min; MS (ESIneg): m/z=233.1 [M−H]⁻

### Intermediate 37

### 4,7-dichloro-1-methyl-2-oxo-1,2-dihydroquinoline-3-carbonitrile

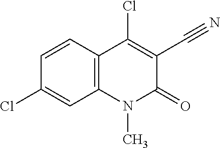 [SMILES: Cn1c(=O)c(C#N)c(Cl)c2ccc(Cl)cc21]

A mixture of 2.48 g 7-chloro-4-hydroxy-1-methyl-2-oxo-1,2-dihydroquinoline-3-carbonitrile (10.6 mmol, intermediate 36) and 15 mL phosphoric trichloride (160 mmol) was stirred overnight at 90° C. After cooling to rt, dichloromethane (20 ml) and ice water (500 ml) were added and the mixture was extracted with dichloromethane (3×). The combined organic phases were washed with brine, filtered (using a waterresistant filter) and concentrated under reduced pressure to give 2.46 g of the title compound (95% purity, 87% yield).

¹H NMR (DMSO-d₆) δ: 8.07 (d, 1H), 7.85 (d, 1H), 7.55 (dd, 1H), 3.65 (s, 3H).

LC-MS (Method 1): Rₜ=1.10 min; MS (ESIpos): m/z=253.1 [M+H]⁺

### Intermediate 38

### 1,7-dimethyl-2H-3,1-benzoxazine-2,4(1H)-dione

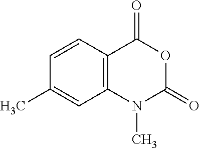

To a solution of 4.78 g 7-methyl-2H-3,1-benzoxazine-2,4(1H)-dione (27 mmol, CAS 63480-11-5) and 9.4 mL N,N-diisopropylethylamine (54 mmol) in 45 mL DMF was added 5.1 mL iodomethane (81 mmol) and was stirred overnight at rt. To the mixture was added ice water (300 ml). The solid that precipitated from this procedure was collected by filtration, washed with water and hexane and dried in vacuum to give 4.92 g of the title compound (95% purity, 91% yield).

¹H NMR (DMSO-d₆) δ: 7.89 (d, 1H), 7.29 (s, 1H), 7.15-7.19 (m, 1H), 3.45 (s, 3H), 2.46 (s, 3H).

LC-MS (Method 1): Rₜ=0.87 min; MS (ESIpos): m/z=192.1 [M+H]⁺

### Intermediate 39

### 4-hydroxy-1,7-dimethyl-2-oxo-1,2-dihydroquinoline-3-carbonitrile

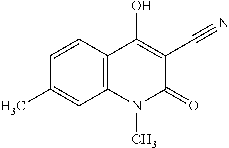 [SMILES: Cc1ccc2c(O)c(C#N)c(=O)n(C)c2c1]

A suspension of 4.8 g 1,7-dimethyl-2H-3,1-benzoxazine-2,4(1H)-dione (25.1 mmol, intermediate 38) in 65 mL 2-methyltetrahydrofuran was slowly treated with 28 mL triethylamine (200 mmol) followed by the addition of 11 mL ethyl cyanoacetate (100 mmol) and was stirred for 113 h at 80° C. After cooling to rt, the solvent was evaporated in vacuum, water/ethyl acetate (200 ml, 1:1) was added and the mixture was acidified to pH=1 by addition of hydrochloric acid (2 M). The solid that precipitated from this procedure was collected by filtration, washed with water, ethyl acetate and hexane and dried in vacuum to give 5.55 g of the title compound (98% purity, 101% yield).

¹H NMR (DMSO-d₆) δ: 7.97 (d, 1H), 7.38 (s, 1H), 7.16 (dd, 1H), 3.54 (s, 3H), 2.46 (s, 3H).

LC-MS (Method 1): Rₜ=0.72 min; MS (ESIpos): m/z=215.2 [M+H]⁺

### Intermediate 40

### 4-chloro-1,7-dimethyl-2-oxo-1,2-dihydroquinoline-3-carbonitrile

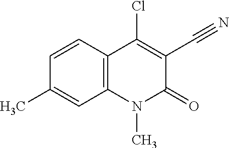 [SMILES: Cc1ccc2c(Cl)c(C#N)c(=O)n(C)c2c1]

A mixture of 5.45 g 4-hydroxy-1,7-dimethyl-2-oxo-1,2-dihydroquinoline-3-carbonitrile (24.9 mmol, intermediate 39) and 35 mL phosphoric trichloride (370 mmol) was stirred overnight at 90° C. After cooling to rt, ice water (400 ml) was added and the mixture was extracted with dichloromethane (3×). The combined organic phases were washed with brine, filtered (using a waterresistant filter) and concentrated under reduced pressure. The residue was stirred in dichloromethane and ethanol, the precipitate was collected by filtration, washed with dichloromethane and dried in vacuum. 2.4 g of the title compound were obtained (31% yield, 74% purity).

¹H NMR (DMSO-d₆) δ: 7.90-7.99 (m, 1H), 7.51-7.62 (m, 1H), 7.31-7.35 (m, 1H), 3.61-3.68 (m, 3H), 2.52-2.54 (m, 3H).

LC-MS (Method 1): Rₜ=1.07 min; MS (ESIpos): m/z=233.2 [M+H]⁺

### Intermediate 41

### methyl [1-(3-cyano-1-methyl-2-oxo-1,2-dihydroquinolin-4-yl)piperidin-4-yl]acetate

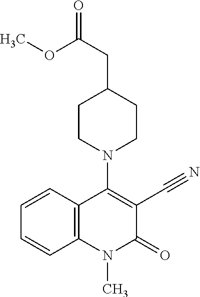 [SMILES: COC(=O)CC1CCN(c2c(C#N)c(=O)n(C)c3ccccc23)CC1]

To a suspension of 500 mg 4-chloro-1-methyl-2-oxo-1,2-dihydroquinoline-3-carbonitrile (2.29 mmol, CAS 150617-68-8, synthesis described in WO2012009649, example 1—compound Ill) and 1.2 mL N,N-diisopropylethylamine (6.9 mmol) in 10 mL 2-propanol was added 431 mg methyl (piperidin-4-yl)acetate (2.74 mmol, CAS 168986-49-0) and the reaction was stirred for 2 h at 90° C. After this time, water was added, the precipitate was collected by filtration, washed with ethanol and dried in vacuum to give 620 mg of the title compound (80% yield).

¹H NMR (DMSO-d₆) δ: 7.78-7.87 (m, 1H), 7.68-7.77 (m, 1H), 7.50-7.58 (m, 1H), 7.27-7.36 (m, 1H), 3.69-3.79 (m, 2H), 3.60-3.66 (m, 3H), 3.51-3.60 (m, 3H), 3.36-3.43 (m, 2H), 2.35-2.41 (m, 2H), 1.95-2.13 (m, 1H), 1.79-1.87 (m, 2H), 1.45-1.58 (m, 2H).

LC-MS (Method 1): Rₜ=1.07 min; MS (ESIpos): m/z=341 [M+H]⁺

### Intermediate 42

### [1-(3-cyano-1-methyl-2-oxo-1,2-dihydroquinolin-4-yl)piperidin-4-yl]acetic acid

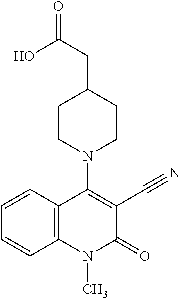 [SMILES: Cn1c(=O)c(C#N)c(N2CCC(CC(=O)O)CC2)c2ccccc21]

To 617 mg methyl [1-(3-cyano-1-methyl-2-oxo-1,2-dihydroquinolin-4-yl)piperidin-4-yl]acetate (1.82 mmol, intermediate 41) in 50 mL THF and 12 mL ethanol was added 11 mL lithium hydroxide (1.0 M, 11 mmol) and the mixture was stirred for 3 h at rt. The reaction was diluted with water and adjusted to pH=3-4 with hydrogen chloride (4M). The organic solvents were evaporated, the precipitate was collected by filtration, washed with water and dried in vacuum to give 538 mg of the title compound (95% purity, 86% yield).

¹H NMR (DMSO-d₆) δ: 12.29 (br s, 1H), 7.65-7.90 (m, 2H), 7.46-7.64 (m, 1H), 7.24-7.38 (m, 1H), 3.69-3.80 (m, 2H), 3.57 (s, 3H), 3.38-3.44 (m, 2H), 2.24-2.31 (m, 2H), 1.94-2.11 (m, 1H), 1.81-1.90 (m, 2H), 1.41-1.59 (m, 2H).

LC-MS (Method 1): Rₜ=0.89 min; MS (ESIpos): m/z=326 [M+H]⁺

### Intermediate 43

### (rac)-tert-butyl 4-[(methanesulfonyl)oxy]azepane-1-carboxylate

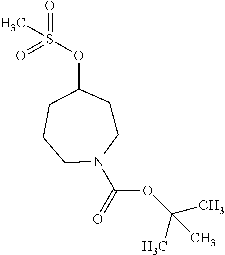 [SMILES: CC(C)(C)OC(=O)N1CCCC(OS(C)(=O)=O)CC1]

To a solution of 2 g (rac)-tert-butyl 4-hydroxyazepane-1-carboxylate (9.29 mmol, CAS 478832-21-2) and 2.8 mL triethylamine (20 mmol) in 28 mL THF at 0° C. was added slowly 790 μL methanesulfonyl chloride (10 mmol) and the reaction was stirred for 6 h at 0° C. The mixture was poured into an aqueous solution of bicarbonate and extracted with ethyl acetate (3×). The combined organic phases were washed with water and brine, dried and concentrated under reduced pressure to give 2 g of the title compound (95% purity, 70% yield).

¹H NMR (DMSO-d₆) δ: 4.74-4.85 (m, 1H), 3.28-3.43 (m, 4H), 3.13-3.19 (m, 3H), 1.93-2.03 (m, 1H), 1.73-1.90 (m, 4H), 1.56-1.68 (m, 1H), 1.36-1.43 (m, 9H).

### Intermediate 44

### (rac)-tert-butyl 4-[4-(trifluoromethoxy)phenoxy]azepane-1-carboxylate

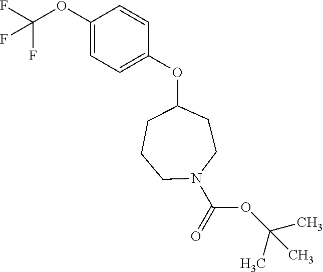 [SMILES: CC(C)(C)OC(=O)N1CCCC(Oc2ccc(OC(F)(F)F)cc2)CC1]

To a solution of 300 mg (rac)-tert-butyl 4-[(methanesulfonyl)oxy]azepane-1-carboxylate (971 μmol, intermediate 43) and 633 mg cesium carbonate (1.94 mmol) in 10 mL DMF was added 170 μL 4-(trifluoromethoxy)phenol (1.2 mmol, CAS 828-27-3) and the reaction was stirred for 6 h at 90° C. The mixture was poured into an aqueous solution of bicarbonate and extracted with ethyl acetate (2×). The combined organic phases were washed with brine, dried and concentrated under reduced pressure. The residue was purified by flash chromatography (silica, hexane/ethyl acetate gradient 0-20%) to give 260 mg of the title compound (95% purity, 68% yield).

¹H NMR (DMSO-d₆) δ: 7.23-7.33 (m, 2H), 6.96-7.04 (m, 2H), 4.44-4.59 (m, 1H), 3.35-3.47 (m, 2H), 3.23-3.32 (m, 1H), 1.92-2.05 (m, 1H), 1.69-1.88 (m, 4H), 1.54-1.65 (m, 1H), 1.38-1.43 (m, 9H).

### Intermediate 45

### (rac)-4-[4-(trifluoromethoxy)phenoxy]azepane

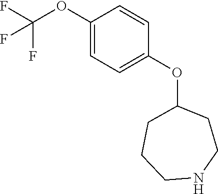 [SMILES: FC(F)(F)Oc1ccc(OC2CCCNCC2)cc1]

To a solution of 255 mg (rac)-tert-butyl 4-[4-(trifluoromethoxy)phenoxy]azepane-1-carboxylate (655 μmol, intermediate 44) in 4.2 mL dichloromethane was added 500 μL trifluoroacetic acid (6.5 mmol) and the mixture was stirred overnight at rt. The mixture was concentrated under reduced pressure and the residue was diluted with toluene. The solvent was evaporated to give 307 mg TFA salt of the title compound (95% purity, 164% yield).

¹H NMR (DMSO-d₆) δ: 7.25-7.34 (m, 2H), 6.99-7.09 (m, 2H), 4.67-4.76 (m, 1H), 3.08-3.32 (m, 4H), 2.10-2.22 (m, 1H), 1.97-2.07 (m, 2H), 1.80-1.94 (m, 2H), 1.66-1.78 (m, 1H).

LC-MS (Method 1): Rₜ=1.26 min; MS (ESIpos): m/z=376.5 [M+H]⁺

### Intermediate 46

### (rac)-tert-butyl 4-(4-bromophenoxy)azepane-1-carboxylate

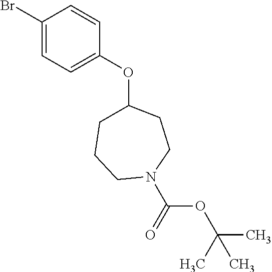 [SMILES: CC(C)(C)OC(=O)N1CCCC(Oc2ccc(Br)cc2)CC1]

To a solution of 1 g (rac)-tert-butyl 4-[(methanesulfonyl)oxy]azepane-1-carboxylate (3.24 mmol, intermediate 43) and 2.11 g cesium carbonate (6.48 mmol) in 33 mL DMF was added 708 mg 4-bromophenol (3.89 mmol, CAS 106-41-2) and the reaction was stirred for 3 h at 90° C. The mixture was poured into an aqueous solution of bicarbonate and extracted with ethyl acetate (2×). The combined organic phases were washed with brine, dried and concentrated under reduced pressure. The residue was purified by flash chromatography (silica, hexane/ethyl acetate gradient 0-15%) to give 540 mg of the title compound (95% purity, 43% yield).

¹H NMR (DMSO-d₆) δ: 7.35-7.49 (m, 2H), 6.85-6.94 (m, 2H), 4.40-4.59 (m, 1H), 3.34-3.46 (m, 2H), 3.21-3.31 (m, 1H), 1.89-2.03 (m, 1H), 1.66-1.85 (m, 4H), 1.52-1.64 (m, 1H), 1.40 (s, 9H).

### Intermediate 47

### (rac)-4-(4-bromophenoxy)azepane

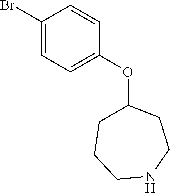 [SMILES: Brc1ccc(OC2CCCNCC2)cc1]

To a solution of 535 mg tert-butyl (rac)-4-(4-bromophenoxy)azepane-1-carboxylate (1.37 mmol, intermediate 46) in 8.8 mL dichloromethane was added 1.1 mL trifluoroacetic acid (14 mmol) and the mixture was stirred overnight at rt. The mixture was concentrated under reduced pressure and the residue was diluted with toluene. The solvent was evaporated to give 550 mg TFA salt of the title compound (95% purity, 141% yield).

¹H NMR (DMSO-d₆) δ: 7.39-7.56 (m, 2H), 6.86-6.99 (m, 2H), 4.57-4.78 (m, 1H), 3.05-3.30 (m, 4H), 1.63-2.22 (m, 6H).

LC-MS (Method 2): Rₜ=1.19 min; MS (ESIpos): m/z=270.4 [M+H]⁺

### Intermediate 48

### 4-chloro-6-methoxy-1-methyl-2-oxo-1,2-dihydroquinoline-3-carbonitrile

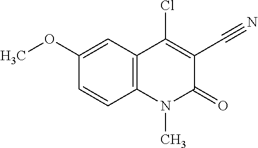 [SMILES: COc1ccc2c(c1)c(Cl)c(C#N)c(=O)n2C]

A mixture of 2.7 g 4-hydroxy-6-methoxy-1-methyl-2-oxo-1,2-dihydroquinoline-3-carbonitrile (11.7 mmol, intermediate 68) and 11 mL phosphoric trichloride (120 mmol) was stirred for 10 h at 90° C. After cooling to rt, ice water (2000 ml) w as added carefully and the mixture was adjusted to pH=10 with sodium carbonate. The precipitate was collected by filtration to obtain 2.6 g of a crude solid. This solid was purified by flash chromatography (silica, dichloromethane/methanol gradient 0-3%) to give 700 mg of the title compound (23% yield).

¹H NMR (400 MHz, DMSO-d₆) δ ppm 3.66 (s, 3H); 3.89 (s, 3H); 7.42 (d, 1H); 7.56 (dd, 1H); 7.70 (d, 1H).

LC-MS (Method 2): Rₜ=0.80 min; MS (ESIpos): m/z=249.3 [M+H]⁺

### Intermediate 49

### 4,6-dichloro-1-methyl-2-oxo-1,2-dihydroquinoline-3-carbonitrile

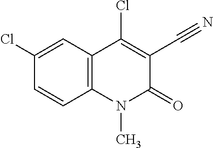 [SMILES: Cn1c(=O)c(C#N)c(Cl)c2cc(Cl)ccc21]

Intermediate 49 was isolated as a by-product of the synthesis of intermediate 48. After flash chromatography (silica, dichloromethane/methanol gradient 0-3%) 180 mg of the title compound (6% yield) were obtained.

¹H NMR (400 MHz, DMSO-d₆) δ ppm 4.00 (s, 3H); 7.51 (d, 1H); 7.75 (dd, 1H); 8.06 (d, 1H).

### Intermediate 50

### 4-[4-(hydroxymethyl)piperidin-1-yl]-1-methyl-2-oxo-1,2-dihydroquinoline-3-carbonitrile

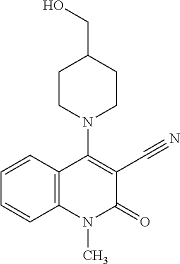 [SMILES: Cn1c(=O)c(C#N)c(N2CCC(CO)CC2)c2ccccc21]

To a solution of 800 mg 4-chloro-1-methyl-2-oxo-1,2-dihydroquinoline-3-carbonitrile (3.00 g, 3.29 mmol, CAS 150617-68-8, synthesis described in WO2012009649, example 1—compound III) and 1.7 mL N,N-diisopropylethylamine (9.9 mmol) in 17 mL 2-propanol was added 455 mg (piperidin-4-yl)methanol (3.95 mmol, CAS 6457-49-4) and the reaction was stirred for 2 h at 90° C. After this time, water was added and the precipitate was collected by filtration, washed with ethanol and dried in vacuum to give 920 mg of the title compound (95% purity, 89% yield).

¹H NMR (DMSO-d₆) δ: 7.78-7.88 (m, 1H), 7.70-7.78 (m, 1H), 7.52-7.60 (m, 1H), 7.28-7.40 (m, 1H), 4.54-4.64 (m, 1H), 3.70-3.85 (m, 2H), 3.28-3.44 (m, 7H), 1.79-1.92 (m, 2H), 1.61-1.76 (m, 1H), 1.36-1.54 (m, 2H).

LC-MS (Method 1): Rₜ=0.84 min; MS (ESIpos): m/z=298 [M+H]⁺

### Intermediate 51

### 8-fluoro-1-methyl-2H-3,1-benzoxazine-2,4(1H)-dione

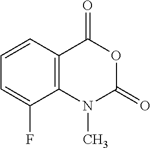

To a suspension of 5 g 8-fluoro-2H-3,1-benzoxazine-2,4(1H)-dione (26.2 mmol, CAS 174463-53-7) in 200 mL DMF at 0° C. was added 2.4 mL iodomethane (39 mmol) and 1.45 g sodium hydride (60% in mineral oil, 39.3 mmol) and the mixture was stirred overnight at rt. After that time, water and ethyl acetate was added and the mixture was extracted with ethyl acetate (2×). The combined organic phases were washed with brine and water, dried and concentrated under reduced pressure. The residue was purified by flash chromatography (silica, hexane/ethyl acetate gradient 0-25%) to give 2.40 g of the title compound (95% purity, 45% yield).

¹H NMR (DMSO-d₆) δ: 7.83-7.96 (m, 1H), 7.70-7.80 (m, 1H), 7.27-7.42 (m, 1H), 3.57-3.68 (m, 3H).

### Intermediate 52

### 8-fluoro-4-hydroxy-1-methyl-2-oxo-1,2-dihydroquinoline-3-carbonitrile

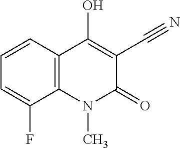 [SMILES: Cn1c(=O)c(C#N)c(O)c2cccc(F)c21]

A suspension of 2.4 g 8-fluoro-1-methyl-2H-3,1-benzoxazine-2,4(1H)-dione (11.7 mmol, intermediate 51) in 27 mL THF was slowly treated with 13 mL triethylamine (93 mmol) followed by the addition of 1.9 mL ethyl cyanoacetate (18 mmol) and was stirred for 24 h at 70° C. After cooling to rt, the solvent was evaporated in vacuum, ethyl acetate and water were added and the mixture was acidified to pH=1 by addition of hydrochloric acid (2 N). The solid that precipitated from this procedure was collected by filtration, washed with water and ethyl acetate and dried in vacuum to give 2.4 g of the title compound (95% purity, 89% yield).

¹H NMR (DMSO-d₆) δ: 7.83-7.98 (m, 1H), 7.53-7.68 (m, 1H), 7.26 (td, 1H), 3.62-3.81 (m, 3H).

LC-MS (Method 2): Rₜ=0.49 min; MS (ESIpos): m/z=219.3 [M+H]⁺

### Intermediate 53

### 4-chloro-8-fluoro-1-methyl-2-oxo-1,2-dihydroquinoline-3-carbonitrile

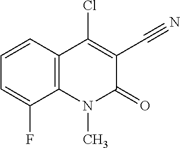 [SMILES: Cn1c(=O)c(C#N)c(Cl)c2cccc(F)c21]

A suspension of 1.8 g 8-fluoro-4-hydroxy-1-methyl-2-oxo-1,2-dihydroquinoline-3-carbonitrile (7.84 mmol, intermediate 52) and 7.3 mL phosphoric trichloride (78 mmol) was stirred for 20 h at 90° C. After cooling to rt, ice water was added carefully and the mixture was adjusted to pH=10 with sodium carbonate. The precipitate was collected by filtration to give 1.5 g of the title compound (95% purity, 77% yield).

¹H NMR (DMSO-d₆) δ: 7.91-8.02 (m, 1H), 7.74-7.87 (m, 1H), 7.44-7.53 (m, 1H), 3.76-3.84 (m, 3H).

LC-MS (Method 2): Rₜ=0.97 min; MS (ESIpos): m/z=237.3 [M+H]⁺

### Intermediate 54

### 8-bromo-1-methyl-2H-3,1-benzoxazine-2,4(1H)-dione

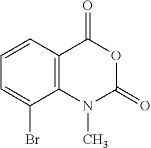

To a suspension of 90 g 8-bromo-2H-3,1-benzoxazine-2,4(1H)-dione (372 mmol, CAS 331646-98-1) and 130 mL N,N-diisopropylethylamine (740 mmol) in 720 mL dimethylacetamide was added 69 mL iodomethane (1.1 mol) and the mixture was stirred overnight at rt. After that time, water was added and the precipitate was collected by filtration, washed with ethanol and hexane to give 86 g of the title compound (90% yield).

¹H NMR (400 MHz, DMSO-d₆) δ ppm 3.64 (s, 3H); 7.26 (t, 1H); 8.00 (dd, 1H); 8.09 (dd, 1H).

LC-MS (Method 1): Rₜ=0.92 min; MS (ESIpos): m/z=256.1 [M+H]⁺

### Intermediate 55

### 8-bromo-4-hydroxy-1-methyl-2-oxo-1,2-dihydroquinoline-3-carbonitrile

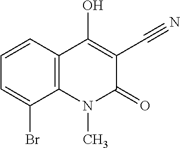 [SMILES: Cn1c(=O)c(C#N)c(O)c2cccc(Br)c21]

A suspension of 40 g 8-bromo-1-methyl-2H-3,1-benzoxazine-2,4(1H)-dione (156 mmol, intermediate 54) in 320 mL THF was slowly treated with 170 mL triethylamine (1.2 mol) followed by the addition of 50 mL ethyl cyanoacetate (470 mmol) and was stirred for 24 h at 70° C. After cooling to rt, the solvent was evaporated in vacuum, water was added and the mixture was acidified to pH=1 by addition of hydrochloric acid (2 N). The mixture was extracted with ethyl acetate (3×). The combined organic phases were washed with brine, dried and concentrated under reduced pressure. The residue was stirred in cyclopentyl methyl ether, the solid that precipitated from this procedure was collected by filtration to give 38.6 g of the title compound (89% yield).

¹H NMR (DMSO-d₆) δ: 8.22-8.98 (m, 1H), 7.83-8.08 (m, 2H), 7.12-7.29 (m, 1H), 3.62-3.74 (m, 3H).

### Intermediate 56

### 8-bromo-4-chloro-1-methyl-2-oxo-1,2-dihydroquinoline-3-carbonitrile

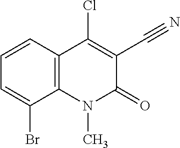 [SMILES: Cn1c(=O)c(C#N)c(Cl)c2cccc(Br)c21]

A suspension of 35.6 g 8-bromo-4-hydroxy-1-methyl-2-oxo-1,2-dihydroquinoline-3-carbonitrile (127 mmol, intermediate 55) and 120 mL phosphoric trichloride (1.3 mol) was stirred for 134 h at 90° C. After cooling to rt, hexane was added and the precipitate was collected by filtration. The solid was stirred with an aqueous solution of sodium carbonate, filtered, washed with ethyl acetate and dried in vacuum. The impure product was solved in dichloromethane, the organic phase was washed with brine, dried and concentrated under reduced pressure. The residue was stirred in dichloromethane, the precipitate was collected by filtration and dried in vacuum to give 6.12 g of the title compound (16% yield).

¹H NMR (DMSO-d₆) δ: 8.08-8.31 (m, 2H); 7.35-7.44 (m, 1H); 3.74-3.87 (m, 3H).

### Intermediate 57

### 8-chloro-1-methyl-2H-3,1-benzoxazine-2,4(1H)-dione

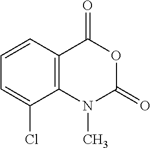

To a solution of 4 g 8-chloro-2H-3,1-benzoxazine-2,4(1H)-dione (19.2 mmol, CAS 63497-60-9) and 6.7 mL N,N-diisopropylethylamine (38 mmol) in 33 mL DMF was added 3.6 mL iodomethane (58 mmol) and was stirred overnight at rt. To the mixture was added ice water (300 ml). The solid that precipitated from this procedure was collected by filtration, washed with water and hexane and dried in vacuum to give 4.4 g of the title compound (90% purity, 97% yield).

¹H NMR (DMSO-d₆) δ: 7.98 (dd, 1H); 7.92 (dd, 1H); 7.34 (t, 1H); 3.64 (s, 3H).

LC-MS (Method 1): Rₜ=0.92 min; MS (ESIpos): m/z=212.1 [M+H]⁺

### Intermediate 58

### 8-chloro-4-hydroxy-1-methyl-2-oxo-1,2-dihydroquinoline-3-carbonitrile

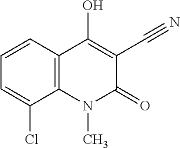 [SMILES: Cn1c(=O)c(C#N)c(O)c2cccc(Cl)c21]

A suspension of 4.35 g 8-chloro-1-methyl-2H-3,1-benzoxazine-2,4(1H)-dione (18.5 mmol, intermediate 57) in 45 mL 2-methyltetrahydrofuran was slowly treated with 21 mL triethylamine (150 mmol) followed by the addition of 9.7 mL ethyl cyanoacetate (74 mmol) and was stirred for 138 h at 80° C. After cooling to rt, the solvent was evaporated in vacuum, water/ethyl acetate (200 ml, 1:1) was added and the mixture was acidified to pH=1 by addition of hydrochloric acid (2 M). The solid that precipitated from this procedure was collected by filtration, washed with water, ethyl acetate and hexane and dried in vacuum to give 3.87 g of the title compound (99% purity, 88% yield).

¹H NMR (DMSO-d₆) δ: 8.02 (dd, 1H); 7.75 (dd, 1H); 7.25 (t, 1H); 3.68 (s, 3H).

LC-MS (Method 1): Rₜ=0.74 min; MS (ESIneg): m/z=233.2 [M−H]⁻

### Intermediate 59

### 4,8-dichloro-1-methyl-2-oxo-1,2-dihydroquinoline-3-carbonitrile

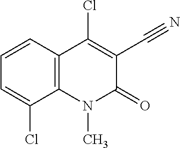 [SMILES: Cn1c(=O)c(C#N)c(Cl)c2cccc(Cl)c21]

A mixture of 3.8 g 8-chloro-4-hydroxy-1-methyl-2-oxo-1,2-dihydroquinoline-3-carbonitrile (16.2 mmol, intermediate 58) and 23 mL phosphoric trichloride (240 mmol) was stirred overnight at 90° C. After cooling to rt, ice water (400 ml) was added and the mixture was extracted with dichloromethane (3×). The combined organic phases were washed with brine, filtered (using a waterresistant filter) and concentrated under reduced pressure. The residue was stirred in dichloromethane/ethanol (1:1, 50 ml), the precipitate was collected by filtration and dried in vacuum to give 1.66 g of the title compound (38% yield, 94% purity).

¹H NMR (DMSO-d₆) δ: 8.09 (dd, 1H); 8.00 (dd, 1H); 7.48 (t, 1H); 3.81 (s, 3H).

LC-MS (Method 1): Rₜ=1.08 min; MS (ESIpos): m/z=253.1 [M+H]⁺

### Intermediate 60

### 6-bromo-1-methyl-2H-3,1-benzoxazine-2,4(1H)-dione

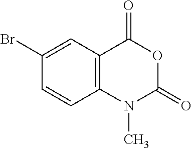

To a solution of 65 g 6-bromo-2H-3,1-benzoxazine-2,4(1H)-dione (269 mmol, CAS 4692-98-2) and 94 mL N,N-diisopropylethylamine (540 mmol) in 520 mL dimethylacetamide was added 50 mL iodomethane (810 mmol) at rt and was stirred overnight. To the reaction was added 2000 mL water. The solid that precipitated from this procedure was collected by filtration, washed with water, ethanol and hexane and dried in an oven at 50° C. 53.7 g of the title compound were obtained (78% yield).

¹H NMR (DMSO-d₆) δ: 7.95-8.12 (m, 2H), 7.37-7.49 (m, 1H), 3.46 (s, 3H).

LC-MS (Method 1): Rₜ=0.93 min; MS (ESIneg): m/z=253.3 [M−H]⁻

### Intermediate 61

### 6-bromo-4-hydroxy-1-methyl-2-oxo-1,2-dihydroquinoline-3-carbonitrile

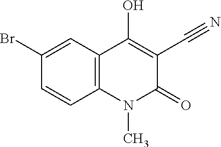 [SMILES: Cn1c(=O)c(C#N)c(O)c2cc(Br)ccc21]

A suspension of 53.7 g 6-bromo-1-methyl-2H-3,1-benzoxazine-2,4(1H)-dione (210 mmol, intermediate 60) in 430 mL THF was slowly treated with 230 mL triethylamine (1.7 mol) followed by the addition of 67 mL ethyl cyanoacetate (630 mmol) and was stirred overnight at 70° C. After cooling to rt, the solvent was evaporated in vacuum, water was added and the mixture was acidified to pH=1 by addition of hydrochloric acid. The mixture was extracted with ethyl acetate (3×). The combined organic phases were washed with brine, dried over sodium sulfate and concentrated under reduced pressure. The residue was stirred in hexane/ethyl acetate, the solid that precipitated from this procedure was collected by filtration to give 61 g of the title compound.

¹H NMR (DMSO-d₆) δ: 8.12-8.19 (m, 1H); 7.82-7.88 (m, 1H); 7.43-7.51 (m, 1H); 4.01 (s, 3H).

LC-MS (Method 1): Rₜ=0.70 min; MS (ESIneg): m/z=277.4 [M−H]⁻

### Intermediate 62

### 6-bromo-4-chloro-1-methyl-2-oxo-1,2-dihydroquinoline-3-carbonitrile

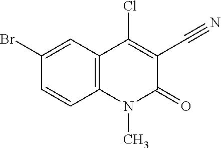 [SMILES: Cn1c(=O)c(C#N)c(Cl)c2cc(Br)ccc21]

A mixture of 58 g 6-bromo-4-hydroxy-1-methyl-2-oxo-1,2-dihydroquinoline-3-carbonitrile (208 mmol, intermediate 61) and 200 mL phosphoric trichloride (2.1 mol) was stirred overnight at 90° C. After cooling to rt, hexane was added and the precipitate was collected by filtration. The solid was stirred with an aqueous solution of sodium carbonate, filtered, washed with ethyl acetate and dried in vacuum. The impure product was stirred in ethanol/acetonitrile (1:1, 200 ml), the precipitate was collected by filtration and dried in vacuum to give 40.4 g of the title compound (65% yield).

¹H NMR (DMSO-d₆) δ: 8.11-8.19 (m, 1H); 7.97-8.11 (m, 1H); 7.65-7.72 (m, 1H); 3.65 (s, 3H).

LC-MS (Method 1): Rₜ=1.11 min; MS (ESIpos): m/z=297.2 [M+H]⁺

### Intermediate 63

### 4-chloro-6-hydroxy-1-methyl-2-oxo-1,2-dihydroquinoline-3-carbonitrile

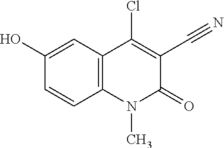 [SMILES: Cn1c(=O)c(C#N)c(Cl)c2cc(O)ccc21]

To a solution of 15 g 4-chloro-6-methoxy-1-methyl-2-oxo-1,2-dihydroquinoline-3-carbonitrile (CAS 1598417-48-1, intermediate 48) in 300 mL dichloromethane at −40° C. was added 75.5 g boron tribromide (29.1 ml). Then the mixture was stirred at 25° C. for 8 h. Full conversion was checked by LC-MS. 150 mL methanol were carefully added at −30° C. After stirring at rt the solvent was removed in vacuum and the residue was stirred with DMSO. The residue was filtered, the collected solid was washed with water and dried in vacuum to generate 6.79 g (48% yield) of the title compound as yellow solid.

¹H-NMR (400 MHz, DMSO-d6) δ[ppm]: 10.15 (s, 1H); 7.59 (d, 1H); 7.38-7.33 (m, 2H); 3.62 (s, 3H).

### Intermediate 64

### 8-bromo-1,6-dimethyl-2H-3,1-benzoxazine-2,4(1H)-dione

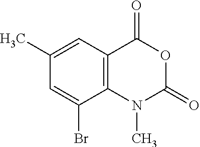

To a solution of 10 g 8-bromo-6-methyl-2H-3,1-benzoxazine-2,4(1H)-dione (39.1 mmol, CAS 177970-27-3) and 14 mL N,N-diisopropylethylamine (78 mmol) in 100 mL dimethylacetamide was added 7.3 mL iodomethane (120 mmol) at rt and was stirred overnight. To the reaction was added 2000 mL water. The solid that precipitated from this procedure was collected by filtration, washed with water, ethanol and hexane and dried in vacuum. 7.2 g of the title compound were obtained (95% purity, 65% yield).

¹H NMR (DMSO-d₆) δ: 7.91-7.99 (m, 1H), 7.79-7.85 (m, 1H), 3.63 (s, 3H), 2.34 (s, 3H).

### Intermediate 65

### 8-bromo-4-hydroxy-1,6-dimethyl-2-oxo-1,2-dihydroquinoline-3-carbonitrile

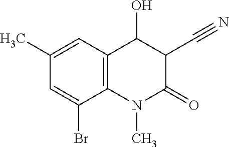 [SMILES: Cc1cc(Br)c2c(c1)C(O)C(C#N)C(=O)N2C]

A suspension of 7.2 g 8-bromo-1,6-dimethyl-2H-3,1-benzoxazine-2,4(1H)-dione (25.3 mmol, intermediate 64) in 150 mL THF was slowly treated with 28 mL triethylamine (200 mmol) followed by the addition of 4.1 mL ethyl cyanoacetate (38 mmol) and was stirred for 8 h at 70° C. After cooling to rt, ethyl acetate and water were added and the mixture was acidified to pH=1 by addition of hydrochloric acid (2 N). The mixture was extracted with ethyl acetate (2×). The combined organic phases were washed with brine and water, dried and concentrated under reduced pressure. The residue was stirred in ethyl acetate, the solid that precipitated from this procedure was collected by filtration to give 4.49 g of the title compound (95% purity, 57% yield).

¹H NMR (DMSO-d₆) δ: 7.71-7.90 (m, 2H), 3.55-3.71 (m, 3H), 2.26-2.38 (m, 3H).

LC-MS (Method 2): Rₜ=0.61 min; MS (ESIpos): m/z=294.0 [M+H]⁺

### Intermediate 66

### 8-bromo-4-chloro-1,6-dimethyl-2-oxo-1,2-dihydroquinoline-3-carbonitrile

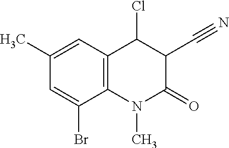 [SMILES: Cc1cc(Br)c2c(c1)C(Cl)C(C#N)C(=O)N2C]

A mixture of 4.49 g 8-bromo-4-hydroxy-1,6-dimethyl-2-oxo-1,2-dihydroquinoline-3-carbonitrile (15.3 mmol, intermediate 65) and 14 mL phosphoric trichloride (150 mmol) was stirred for 10 h at 90° C. After cooling to rt, ice water (1500 ml) was added carefully and the mixture was adjusted to pH=10 with sodium carbonate. The mixture was extracted with dichloromethane (3×). The combined organic phases were washed with water, dried and concentrated under reduced pressure. The residue was purified by flash chromatography (silica, dichloromethane/methanol gradient 0-1%) to give 2.9 g of the title compound (95% purity, 58% yield).

¹H NMR (DMSO-d₆) δ: 8.01-8.11 (m, 1H), 7.89-7.94 (m, 1H), 3.78 (s, 3H), 2.41 (s, 3H).

LC-MS (Method 2): Rₜ=1.19 min; MS (ESIpos): m/z=311.3 [M+H]⁺

### Intermediate 67

### 6-methoxy-1-methyl-2H-3,1-benzoxazine-2,4(1H)-dione

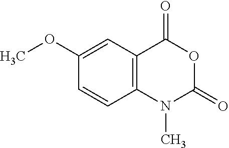

To a solution of 4.6 g 6-methoxy-2H-3,1-benzoxazine-2,4(1H)-dione (22.6 mmol, CAS 37795-77-0) and 7.9 mL N,N-diisopropylethylamine (45 mmol) in 42 mL dimethylacetamide was added 4.2 mL iodomethane (68 mmol) at rt and was stirred overnight. To the reaction was added 1000 mL water. The solid that precipitated from this procedure was collected by filtration, washed with water, ethanol and hexane and dried in vacuum. 4.3 g of the title compound were obtained (87% yield).

¹H NMR (DMSO-d₆) δ ppm 3.45 (s, 3H); 3.84 (s, 3H); 7.39-7.49 (m, 3H).

### Intermediate 68

### 4-hydroxy-6-methoxy-1-methyl-2-oxo-1,2-dihydroquinoline-3-carbonitrile

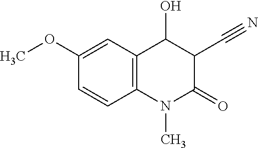 [SMILES: COc1ccc2c(c1)C(O)C(C#N)C(=O)N2C]

A suspension of 4.3 g 6-methoxy-1-methyl-2H-3,1-benzoxazine-2,4(1H)-dione (19.7 mmol, intermediate 67) in 45 mL THF was slowly treated with 22 mL triethylamine (160 mmol) followed by the addition of 3.2 mL ethyl cyanoacetate (30 mmol) and was stirred for 24 h at 70° C. After cooling to rt, ethyl acetate and water were added and the mixture was acidified to pH=1 by addition of hydrochloric acid (2 N). The resulting precipitate was collected by filtration to give 2.7 g of the title compound (57% yield).

¹H NMR (400 MHz, DMSO-d₆) δ ppm 3.53 (s, 3H); 3.83 (s, 3H); 7.38 (dd, 1H); 7.47-7.51 (m, 1H); 7.57 (d, 1H).

LC-MS (Method 2): Rₜ=0.48 min; MS (ESIpos): m/z=231.3 [M+H]⁺

### EXPERIMENTAL SECTION—EXAMPLES

### Example 1

### 1-methyl-4-[4-(4-methylphenoxy)piperidin-1-yl]-2-oxo-1,2-dihydroquinoline-3-carbonitrile

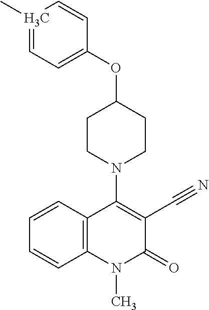 [SMILES: Cc1ccc(OC2CCN(c3c(C#N)c(=O)n(C)c4ccccc34)CC2)cc1]

A solution of 100 mg 4-chloro-1-methyl-2-oxo-1,2-dihydroquinoline-3-carbonitrile (0.5 mmol, CAS 150617-68-8, synthesis described in WO2012009649, example 1—compound III), 105 mg 4-(4-methylphenoxy)piperidine (0.55 mmol, CAS 63843-49-2) and 0.24 mL N,N-diisopropylethylamine (1.4 mmol) in 2 mL 2-propanol was stirred for 2 h at 90° C. After this time, water was added and the reaction was stirred for some time. The residue was collected by filtration and washed with ethanol. The resulting solid was dried at 100° C. in vacuum to give 146 mg of the title compound (81% yield, 95% purity).

¹H NMR (DMSO-d₆) δ: 7.85-7.91 (m, 1H), 7.69-7.78 (m, 1H), 7.53-7.62 (m, 1H), 7.31-7.39 (m, 1H), 7.05-7.14 (m, 2H), 6.87-6.97 (m, 2H), 4.59-4.86 (m, 1H), 3.69-3.82 (m, 2H), 3.49-3.61 (m, 5H), 2.11-2.26 (m, 5H), 1.80-1.98 (m, 2H).

LC-MS (Method 1): Rₜ=1.36 min; MS (ESIpos): m/z=374 [M+H]⁺

### Example 2

### 1-methyl-2-oxo-4-{4-[4-(trifluoromethoxy)phenoxy]piperidin-1-yl}-1,2-dihydroquinoline-3-carbonitrile

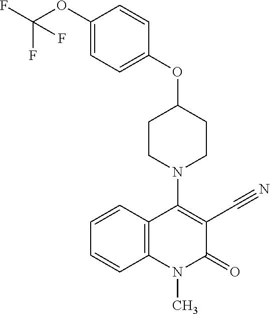 [SMILES: Cn1c(=O)c(C#N)c(N2CCC(Oc3ccc(OC(F)(F)F)cc3)CC2)c2ccccc21]

A solution of 5.0 g 4-chloro-1-methyl-2-oxo-1,2-dihydroquinoline-3-carbonitrile (21.7 mmol, CAS 150617-68-8, synthesis described in WO2012009649, example 1—compound III), 6.57 g 4-[4-(trifluoromethoxy)phenoxy]piperidine (23.9 mmol, CAS 287952-67-4) and 6.1 mL triethylamine (43.5 mmol) in 300 mL 2-propanol was stirred for 5 h at 90° C. After this time, water and ethyl acetate were added and the reaction was extracted with further ethyl acetate.

The combined organic phases were dried over sodium sulfate and the solvent was removed under reduced pressure. Upon evaporation a precipitate was generated and collected by filtration. The resulting solid was dried to obtain 8.1 g of the title compound (80% yield, 95% purity).

¹H NMR (DMSO-d₆) δ: 7.87 (dd, 1H), 7.69-7.78 (m, 1H), 7.53-7.61 (m, 1H), 7.26-7.39 (m, 3H), 7.11-7.20 (m, 2H), 4.70-4.86 (m, 1H), 3.70-3.83 (m, 2H), 3.49-3.64 (m, 5H), 2.13-2.26 (m, 2H), 1.83-2.00 (m, 2H).

LC-MS (Method 2): Rₜ=1.39 min; MS (ESIpos): m/z=444.5 [M+H]⁺

### Example 3

### 1-methyl-4-[4-(3-methylphenoxy)piperidin-1-yl]-2-oxo-1,2-dihydroquinoline-3-carbonitrile

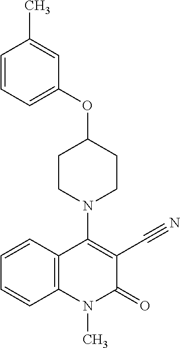 [SMILES: Cc1cccc(OC2CCN(c3c(C#N)c(=O)n(C)c4ccccc34)CC2)c1]

A solution of 100 mg 4-chloro-1-methyl-2-oxo-1,2-dihydroquinoline-3-carbonitrile (0.5 mmol, CAS 150617-68-8, synthesis described in WO2012009649, example 1—compound III), 117 mg 4-(3-methylphenoxy)piperidine (0.52 mmol, CAS 63843-46-9) and 0.12 mL triethylamine (0.87 mmol) in 4.4 mL 2-propanol was stirred for 5 h at 90° C. After this time, water was added and the reaction was extracted with ethyl acetate. The organic phase was washed with water and brine and dried over sodium sulfate. After evaporation of the solvent, the residue was purified by RP-HPLC (column: X-Bridge C18 5 μm 100×30 mm, mobile phase: acetonitrile/water (0.2 vol. % ammonia 32%)-gradient). 23 mg of the title compound were obtained (95% purity, 14% yield).

¹H NMR (DMSO-d₆) δ: 7.82-7.92 (m, 1H), 7.70-7.79 (m, 1H), 7.53-7.62 (m, 1H), 7.31-7.39 (m, 1H), 7.14-7.23 (m, 1H), 6.73-6.90 (m, 3H), 4.66-4.82 (m, 1H), 3.69-3.85 (m, 2H), 3.49-3.61 (m, 5H), 2.25-2.31 (m, 3H), 2.14-2.25 (m, 2H), 1.84-2.00 (m, 2H).

LC-MS (Method 2): Rₜ=1.34 min; MS (ESIpos): m/z=374.5 [M+H]⁺

### Example 4

### 4-[4-(3-methoxyphenoxy)piperidin-1-yl]-1-methyl-2-oxo-1,2-dihydroquinoline-3-carbonitrile

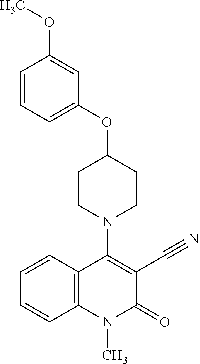 [SMILES: COc1cccc(OC2CCN(c3c(C#N)c(=O)n(C)c4ccccc34)CC2)c1]

A solution of 100 mg 4-chloro-1-methyl-2-oxo-1,2-dihydroquinoline-3-carbonitrile (434 μmol, CAS 150617-68-8, synthesis described in WO2012009649, example 1—compound III), 108 mg 4-(3-methoxyphenoxy)piperidine (521 μmol, CAS 162402-37-1) and 0.12 mL triethylamine (0.87 mmol) in 7.5 mL 2-propanol was stirred for 2.5 h at 90° C. After this time, water was added and the reaction was extracted with ethyl acetate. The organic phase was washed with water and brine and dried over sodium sulfate. After evaporation of the solvent, the residue was purified by RP-HPLC (column: X-Bridge C18 5 μm 100×30 mm, mobile phase: acetonitrile/water (0.2 vol. % ammonia 32%)-gradient). 94 mg of the title compound were obtained (95% purity, 53% yield).

¹H NMR (DMSO-d₆) δ: 7.83-7.91 (m, 1H), 7.69-7.77 (m, 1H), 7.52-7.62 (m, 1H), 7.30-7.39 (m, 1H), 7.15-7.26 (m, 1H), 6.49-6.67 (m, 3H), 4.76 (tt, 1H), 3.70-3.81 (m, 5H), 3.51-3.62 (m, 5H), 2.13-2.25 (m, 2H), 1.84-1.99 (m, 2H).

LC-MS (Method 2): Rₜ=1.26 min; MS (ESIpos): m/z=390.5 [M+H]⁺

### Example 5

### 1-methyl-2-oxo-4-(4-phenoxypiperidin-1-yl)-1,2-dihydroquinoline-3-carbonitrile

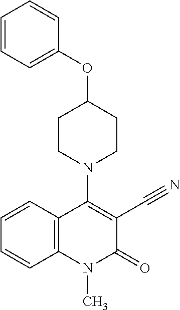 [SMILES: Cn1c(=O)c(C#N)c(N2CCC(Oc3ccccc3)CC2)c2ccccc21]

A suspension of 100 mg 4-chloro-1-methyl-2-oxo-1,2-dihydroquinoline-3-carbonitrile (457 μmol, CAS 150617-68-8, synthesis described in WO2012009649, example 1—compound Ill), 97.3 mg 4-phenoxypiperidine (549 μmol, CAS 3202-33-3) and 0.24 mL N,N-diisopropylethylamine (1.4 mmol) in 2 mL 2-propanol was stirred for 2 h at 90° C. After this time, water was added and the reaction was stirred for some time. The residue was collected by filtration and washed with ethanol. The resulting solid was dried at 100° C. in vacuum to give 142 mg of the title compound (82% yield, 95% purity).

¹H NMR (DMSO-d₆) δ: 7.82-7.94 (m, 1H), 7.70-7.77 (m, 1H), 7.53-7.61 (m, 1H), 7.26-7.39 (m, 3H), 7.01-7.13 (m, 2H), 6.88-6.98 (m, 1H), 4.68-4.84 (m, 1H), 3.71-3.84 (m, 2H), 3.50-3.62 (m, 5H), 2.14-2.25 (m, 2H), 1.85-2.00 (m, 2H).

LC-MS (Method 1): Rₜ=1.28 min; MS (ESIpos): m/z=360 [M+H]⁺

### Example 6

### 4-[4-(4-fluorophenoxy)piperidin-1-yl]-1-methyl-2-oxo-1,2-dihydroquinoline-3-carbonitrile

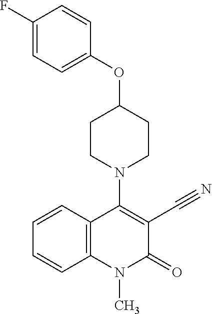 [SMILES: Cn1c(=O)c(C#N)c(N2CCC(Oc3ccc(F)cc3)CC2)c2ccccc21]

A suspension of 100 mg 4-chloro-1-methyl-2-oxo-1,2-dihydroquinoline-3-carbonitrile (457 μmol, CAS 150617-68-8, synthesis described in WO2012009649, example 1—compound Ill), 107 mg 4-(4-fluorophenoxy)piperidine (549 μmol, CAS 3202-34-4) and 0.24 mL N,N-diisopropylethylamine (1.4 mmol) in 2 mL 2-propanol was stirred for 2 h at 90° C. After this time, water was added and the reaction was stirred for some time. The residue was collected by filtration and washed with ethanol. The resulting solid was dried at 100° C. in vacuum to give 135 mg of the title compound (74% yield, 95% purity).

¹H NMR (DMSO-d₆) δ: 7.82-7.91 (m, 1H), 7.69-7.76 (m, 1H), 7.53-7.62 (m, 1H), 7.29-7.38 (m, 1H), 6.98-7.22 (m, 4H), 4.61-4.83 (m, 1H), 3.70-3.82 (m, 2H), 3.47-3.63 (m, 5H), 2.13-2.27 (m, 2H), 1.81-1.99 (m, 2H).

LC-MS (Method 1): Rₜ=1.29 min; MS (ESIpos): m/z=378 [M+H]⁺

### Example 7

### 4-[4-(3-fluorophenoxy)piperidin-1-yl]-1-methyl-2-oxo-1,2-dihydroquinoline-3-carbonitrile

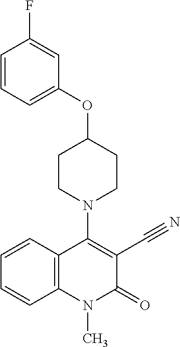 [SMILES: Cn1c(=O)c(C#N)c(N2CCC(Oc3cccc(F)c3)CC2)c2ccccc21]

A suspension of 100 mg 4-chloro-1-methyl-2-oxo-1,2-dihydroquinoline-3-carbonitrile (457 μmol, CAS 150617-68-8, synthesis described in WO2012009649, example 1—compound Ill), 127 mg 4-(3-fluorophenoxy)piperidine hydrogen chloride salt (1:1) (549 μmol, CAS 3202-36-6) and 0.24 mL N,N-diisopropylethylamine (1.4 mmol) in 2 mL 2-propanol was stirred for 2 h at 90° C. After this time, water was added and the reaction was stirred for some time. The residue was collected by filtration and washed with ethanol. The resulting solid was dried at 100° C. in vacuum to give 127 mg of the title compound (70% yield, 95% purity).

¹H NMR (DMSO-d₆) δ: 7.87 (dd, 1H), 7.70-7.79 (m, 1H), 7.57 (d, 1H), 7.27-7.40 (m, 2H), 6.84-7.02 (m, 2H), 6.70-6.80 (m, 1H), 4.72-4.90 (m, 1H), 3.71-3.83 (m, 2H), 3.47-3.63 (m, 5H), 2.13-2.27 (m, 2H), 1.83-1.99 (m, 2H).

LC-MS (Method 1): Rₜ=1.31 min; MS (ESIpos): m/z=378 [M+H]⁺

### Example 8

### 4-[4-(4-bromophenoxy)piperidin-1-yl]-1-methyl-2-oxo-1,2-dihydroquinoline-3-carbonitrile

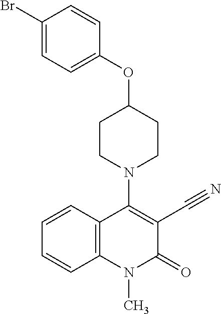 [SMILES: Cn1c(=O)c(C#N)c(N2CCC(Oc3ccc(Br)cc3)CC2)c2ccccc21]

A suspension of 111 mg 4-chloro-1-methyl-2-oxo-1,2-dihydroquinoline-3-carbonitrile (508 μmol, CAS 150617-68-8, synthesis described in WO2012009649, example 1—compound Ill), 130 mg 4-(4-bromophenoxy)piperidine (508 μmol, CAS 74130-05-5) and 0.28 mL triethylamine (2.0 mmol) in 15 mL 2-propanol was stirred for 4 h at 90° C. and 48 h at rt. After this time, water and ethyl acetate were added and the reaction was stirred for some time. The solid was collected by filtration. The impure product was purified by flash chromatography (silica, dichloromethane/methanol gradient 0-2%) to give 66 mg of the title compound (98% purity, 29% yield).

¹H NMR (DMSO-d₆) δ: 7.81-7.92 (m, 1H), 7.70-7.77 (m, 1H), 7.53-7.62 (m, 1H), 7.41-7.51 (m, 2H), 7.34 (td, 1H), 6.98-7.09 (m, 2H), 4.68-4.86 (m, 1H), 3.69-3.83 (m, 2H), 3.50-3.61 (m, 5H), 2.12-2.26 (m, 2H), 1.78-2.02 (m, 2H).

LC-MS (Method 2): Rₜ=1.37 min; MS (ESIpos): m/z=439 [M+H]⁺

### Example 9

### 4-[4-(3-chlorophenoxy)piperidin-1-yl]-1-methyl-2-oxo-1,2-dihydroquinoline-3-carbonitrile

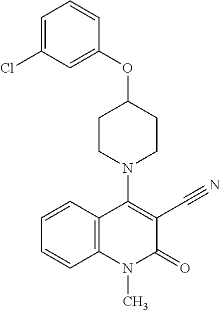 [SMILES: Cn1c(=O)c(C#N)c(N2CCC(Oc3cccc(Cl)c3)CC2)c2ccccc21]

A solution of 100 mg 4-chloro-1-methyl-2-oxo-1,2-dihydroquinoline-3-carbonitrile (457 μmol, CAS 150617-68-8, synthesis described in WO2012009649, example 1—compound III), 107 mg 4-(3-chlorophenoxy)piperidine (503 μmol, CAS 97840-40-9) and 0.13 mL triethylamine (910 μmol) in 6.3 mL 2-propanol was stirred for 5 h at 90° C. After this time, water was added and the reaction was extracted with ethyl acetate. The organic phase was washed with water and brine and dried over sodium sulfate. After evaporation of the solvent, the residue was purified by flash chromatography (silica, dichloromethane/methanol gradient 0-1%). 270 mg of the title compound were obtained (95% purity, 142% yield).

¹H NMR (DMSO-d₆) δ: 7.82-7.97 (m, 1H), 7.67-7.80 (m, 1H), 7.52-7.65 (m, 1H), 7.26-7.42 (m, 2H), 7.12-7.21 (m, 1H), 6.97-7.06 (m, 2H), 4.76-4.92 (m, 1H), 3.71-3.85 (m, 2H), 3.48-3.66 (m, 5H), 2.14-2.27 (m, 2H), 1.80-1.97 (m, 2H).

LC-MS (Method 2): Rₜ=1.35 min; MS (ESIpos): m/z=394.5 [M+H]⁺

### Example 10

### 4-[4-(4-chlorophenoxy)piperidin-1-yl]-1-methyl-2-oxo-1,2-dihydroquinoline-3-carbonitrile

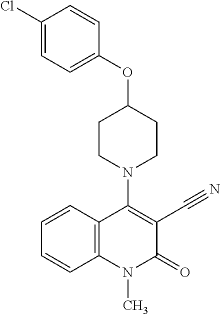 [SMILES: Cn1c(=O)c(C#N)c(N2CCC(Oc3ccc(Cl)cc3)CC2)c2ccccc21]

A solution of 100 mg 4-chloro-1-methyl-2-oxo-1,2-dihydroquinoline-3-carbonitrile (457 μmol, CAS 150617-68-8, synthesis described in WO2012009649, example 1—compound III), 107 mg 4-(4-chlorophenoxy)piperidine (503 μmol, CAS 97839-99-1) and 0.13 mL triethylamine (910 μmol) in 6.3 mL 2-propanol was stirred for 5 h at 90° C. After this time, water was added and the reaction was extracted with ethyl acetate. The organic phase was washed with water and brine and dried over sodium sulfate. After evaporation of the solvent, the residue was purified by flash chromatography (silica, dichloromethane/methanol gradient 0-1%). 175 mg of the title compound were obtained (95% purity, 92% yield).

¹H NMR (DMSO-d₆) δ: 7.83-7.92 (m, 1H), 7.69-7.77 (m, 1H), 7.51-7.61 (m, 1H), 7.39 (s, 3H), 7.02-7.15 (m, 2H), 4.68-4.82 (m, 1H), 3.69-3.84 (m, 2H), 3.46-3.63 (m, 5H), 2.13-2.31 (m, 2H), 1.82-1.99 (m, 2H).

LC-MS (Method 2): Rₜ=1.37 min; MS (ESIpos): m/z=394.5 [M+H]⁺

### Example 11

### 4-[4-(2-methoxyphenoxy)piperidin-1-yl]-1-methyl-2-oxo-1,2-dihydroquinoline-3-carbonitrile

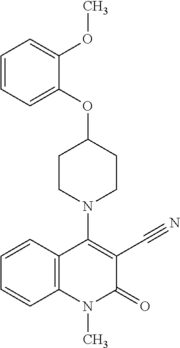 [SMILES: COc1ccccc1OC1CCN(c2c(C#N)c(=O)n(C)c3ccccc23)CC1]

A solution of 100 mg 4-chloro-1-methyl-2-oxo-1,2-dihydroquinoline-3-carbonitrile (434 μmol, CAS 150617-68-8, synthesis described in WO2012009649, example 1—compound III), 108 mg 4-(2-methoxyphenoxy)piperidine (521 μmol, CAS 28033-32-1) and 0.12 mL triethylamine (0.87 mmol) in 7.5 mL 2-propanol was stirred for 2.5 h at 90° C. After this time, water was added and the reaction was extracted with ethyl acetate. The organic phase was washed with water and brine and dried over sodium sulfate. After evaporation of the solvent, the residue was purified by RP-HPLC (column: X-Bridge C18 5 μm 100×30 mm, mobile phase: acetonitrile/water (0.2 vol. % ammonia 32%)-gradient). 102 mg of the title compound were obtained (95% purity, 57% yield).

¹H NMR (DMSO-d₆) δ: 7.84-7.92 (m, 1H), 7.70-7.79 (m, 1H), 7.52-7.62 (m, 1H), 7.31-7.38 (m, 1H), 7.07-7.13 (m, 1H), 6.84-7.03 (m, 3H), 4.56-4.73 (m, 1H), 3.75-3.85 (m, 5H), 3.47-3.62 (m, 5H), 2.10-2.25 (m, 2H), 1.81-2.00 (m, 2H).

LC-MS (Method 2): Rₜ=1.22 min; MS (ESIpos): m/z=390.5 [M+H]⁺

### Example 12

### ethyl 4-{[1-(3-cyano-1-methyl-2-oxo-1,2-dihydroquinolin-4-yl)piperidin-4-yl]oxy}benzoate

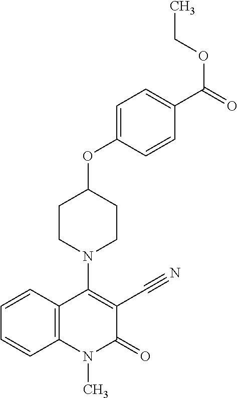 [SMILES: CCOC(=O)c1ccc(OC2CCN(c3c(C#N)c(=O)n(C)c4ccccc34)CC2)cc1]

To a solution of 50 mg 4-(4-hydroxypiperidin-1-yl)-1-methyl-2-oxo-1,2-dihydroquinoline-3-carbonitrile (176 μmol, intermediate 1) and 100 μL ethyl 4-fluorobenzoate (710 μmol, CAS 451-46-7) in 2.0 mL DMF at 0° C. was added 33 mg sodium hydride (60% in mineral oil, 829 μmol) and the mixture was stirred for 5 h at 80° C. The mixture was cooled down to rt, water was added and the reaction was extracted with ethyl acetate (3×). The combined organic layers were filtered and concentrated under reduced pressure. The residue was purified by RP-HPLC (column: Chromatorex 125×30 mm, 10 μm mobile phase: water (0.2 vol. % ammonia 32%)/acetonitrile-gradient) to give 27.3 mg of the title compound (90% purity, 32% yield).

1H-NMR (400 MHz, DMSO-d6): 5 [ppm]=1.31 (t, 3H), 1.88-2.02 (m, 2H), 2.24 (ddd, 2H), 3.51-3.64 (m, 5H), 3.71-3.82 (m, 2H), 4.28 (q, 2H), 4.90 (tt, 1H), 7.11-7.21 (m, 2H), 7.28-7.40 (m, 1H), 7.51-7.62 (m, 1H), 7.74 (ddd, 1H), 7.83-7.96 (m, 3H).

LC-MS (Method 3): Rₜ=1.32 min; MS (ESIpos): m/z=432 [M+H]⁺

### Example 13

### 1-methyl-2-oxo-4-{4-[4-(trifluoromethyl)phenoxy]piperidin-1-yl}-1,2-dihydroquinoline-3-carbonitrile

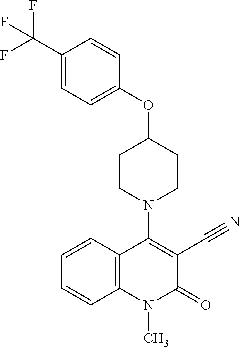 [SMILES: Cn1c(=O)c(C#N)c(N2CCC(Oc3ccc(C(F)(F)F)cc3)CC2)c2ccccc21]

A suspension of 50 mg 4-chloro-1-methyl-2-oxo-1,2-dihydroquinoline-3-carbonitrile (229 μmol, CAS 150617-68-8, synthesis described in WO2012009649, example 1—compound III), 77 mg 4-[4-(trifluoromethyl)phenoxy]piperidine hydrogen chloride salt (1:1) (274 μmol, CAS 287952-09-4) and 0.12 mL N,N-diisopropylethylamine (690 μmol) in 1 mL 2-propanol was stirred for 2 h at 90° C. After this time, water was added and the reaction was stirred for some time. The residue was collected by filtration and washed with ethanol. The resulting solid was dried at 100° C. in vacuum to give 77.9 mg of the title compound (76% yield, 95% purity).

¹H NMR (DMSO-d₆) δ: 7.83-7.92 (m, 1H), 7.70-7.82 (m, 1H), 7.63-7.70 (m, 2H), 7.53-7.61 (m, 1H), 7.29-7.38 (m, 1H), 7.18-7.28 (m, 2H), 4.85-5.02 (m, 1H), 3.70-3.86 (m, 2H), 3.52-3.68 (m, 5H), 2.15-2.30 (m, 2H), 1.87-2.04 (m, 2H).

LC-MS (Method 1): Rₜ=1.39 min; MS (ESIpos): m/z=429 [M+H]⁺

### Example 14

### 1-methyl-4-[4-(2-methylphenoxy)piperidin-1-yl]-2-oxo-1,2-dihydroquinoline-3-carbonitrile

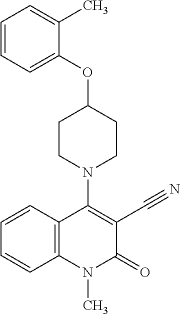 [SMILES: Cc1ccccc1OC1CCN(c2c(C#N)c(=O)n(C)c3ccccc23)CC1]

A solution of 100 mg 4-chloro-1-methyl-2-oxo-1,2-dihydroquinoline-3-carbonitrile (434 μmol, CAS 150617-68-8, synthesis described in WO2012009649, example 1—compound III), 117 mg 4-(2-methylphenoxy)piperidine (521 μmol, CAS 63843-42-5) and 0.12 mL triethylamine (0.87 mmol) in 4.4 mL 2-propanol was stirred for 6 h at 90° C. After this time, water was added and the reaction was extracted with ethyl acetate. The organic phase was washed with water and brine and dried over sodium sulfate. After evaporation of the solvent, the residue was purified by RP-HPLC (column: X-Bridge C18 5 μm 100×30 mm, mobile phase: acetonitrile/water (0.2 vol. % ammonia 32%)-gradient). 75 mg of the title compound were obtained (95% purity, 44% yield).

¹H NMR (DMSO-d₆) δ: 7.84-7.92 (m, 1H), 7.68-7.78 (m, 1H), 7.51-7.63 (m, 1H), 7.28-7.40 (m, 1H), 7.12-7.21 (m, 2H), 7.01-7.10 (m, 1H), 6.81-6.89 (m, 1H), 4.72-4.89 (m, 1H), 3.69-3.89 (m, 2H), 3.50-3.66 (m, 5H), 2.13-2.28 (m, 5H), 1.83-2.04 (m, 2H).

LC-MS (Method 2): Rₜ=1.36 min; MS (ESIpos): m/z=374.5 [M+H]⁺

### Example 15

### 1-methyl-2-oxo-4-{4-[3-(trifluoromethyl)phenoxy]piperidin-1-yl}-1,2-dihydroquinoline-3-carbonitrile

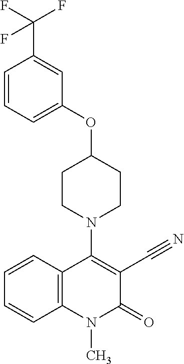 [SMILES: Cn1c(=O)c(C#N)c(N2CCC(Oc3cccc(C(F)(F)F)c3)CC2)c2ccccc21]

A suspension of 100 mg 4-chloro-1-methyl-2-oxo-1,2-dihydroquinoline-3-carbonitrile (457 μmol, CAS 150617-68-8, synthesis described in WO2012009649, example 1—compound Ill), 135 mg 4-[3-(trifluoromethyl)phenoxy]piperidine (549 μmol, CAS 337912-66-0) and 0.24 mL N,N-diisopropylethylamine (1.4 mmol) in 1 mL 2-propanol was stirred for 2 h at 90° C. After this time, water was added and the reaction was stirred for some time. The residue was collected by filtration and washed with ethanol. The resulting solid was dried at 100° C. in vacuum to give 167 mg of the title compound (81% yield, 95% purity).

¹H NMR (DMSO-d₆) δ: 7.85-7.95 (m, 1H), 7.70-7.82 (m, 1H), 7.49-7.62 (m, 2H), 7.26-7.40 (m, 4H), 4.85-5.00 (m, 1H), 3.69-3.85 (m, 2H), 3.50-3.64 (m, 5H), 2.22 (ddd, 2H), 1.87-2.02 (m, 2H).

LC-MS (Method 1): Rₜ=1.40 min; MS (ESIpos): m/z=428 [M+H]⁺

### Example 16

### 4-[4-(2-chlorophenoxy)piperidin-1-yl]-1-methyl-2-oxo-1,2-dihydroquinoline-3-carbonitrile

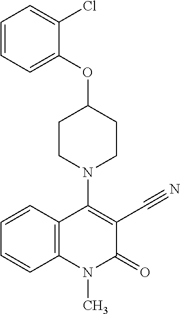 [SMILES: Cn1c(=O)c(C#N)c(N2CCC(Oc3ccccc3Cl)CC2)c2ccccc21]

A solution of 100 mg 4-chloro-1-methyl-2-oxo-1,2-dihydroquinoline-3-carbonitrile (434 μmol, CAS 150617-68-8, synthesis described in WO2012009649, example 1—compound III), 110 mg 4-(2-chlorophenoxy)piperidine (521 μmol, CAS 245057-65-2) and 0.12 mL triethylamine (0.87 mmol) in 7.5 mL 2-propanol was stirred for 3.5 h at 90° C. After this time, water was added and the reaction was extracted with ethyl acetate. The organic phase was washed with water and brine and dried over sodium sulfate. After evaporation of the solvent, the residue was purified by RP-HPLC (column: X-Bridge C18 5 μm 100×30 mm, mobile phase: acetonitrile/water (0.2 vol. % ammonia 32%)-gradient). 75 mg of the title compound were obtained (95% purity, 42% yield).

¹H NMR (DMSO-d₆) δ: 7.82-7.92 (m, 1H), 7.68-7.79 (m, 1H), 7.51-7.61 (m, 1H), 7.41-7.50 (m, 1H), 7.26-7.38 (m, 3H), 6.93-7.03 (m, 1H), 4.80-4.98 (m, 1H), 3.73-3.89 (m, 2H), 3.51-3.62 (m, 5H), 2.16-2.29 (m, 2H), 1.86-2.06 (m, 2H).

LC-MS (Method 2): Rₜ=1.32 min; MS (ESIpos): m/z=394.4 [M+H]⁺

### Example 17

### 1-methyl-2-oxo-4-{4-[4-(1H-1,2,4-triazol-1-yl)phenoxy]piperidin-1-yl}-1,2-dihydroquinoline-3-carbonitrile

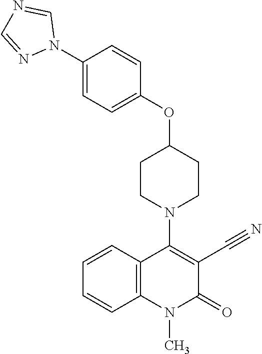 [SMILES: Cn1c(=O)c(C#N)c(N2CCC(Oc3ccc(-n4cncn4)cc3)CC2)c2ccccc21]

A suspension of 157 mg 4-chloro-1-methyl-2-oxo-1,2-dihydroquinoline-3-carbonitrile (716 μmol, CAS 150617-68-8, synthesis described in WO2012009649, example 1—compound Ill), 175 mg 4-[4-(1H-1,2,4-triazol-1-yl)phenoxy]piperidine (716 μmol, intermediate 4) and 0.4 mL triethylamine (2.9 mmol) in 15 mL 2-propanol was stirred for 2 h at 90° C. and 48 h at rt. After this time, water and ethyl acetate were added and the reaction was stirred for some time. The solid was collected by filtration to give 210 mg of the title compound (98% purity, 67% yield).

¹H NMR (DMSO-d₆) δ: 9.15-9.23 (m, 1H), 8.19-8.28 (m, 1H), 7.85-7.94 (m, 1H), 7.70-7.83 (m, 3H), 7.55-7.62 (m, 1H), 7.30-7.40 (m, 1H), 7.18-7.30 (m, 2H), 4.77-4.91 (m, 1H), 3.71-3.85 (m, 2H), 3.51-3.65 (m, 5H), 2.19-2.29 (m, 2H), 1.87-2.05 (m, 2H).

LC-MS (Method 2): Rₜ=1.05 min; MS (ESIpos): m/z=427.6 [M+H]⁺

### Example 18

### 1-methyl-2-oxo-4-{4-[4-(2-oxopyrrolidin-1-yl)phenoxy]piperidin-1-yl}-1,2-dihydroquinoline-3-carbonitrile

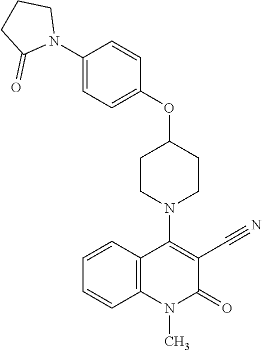 [SMILES: Cn1c(=O)c(C#N)c(N2CCC(Oc3ccc(N4CCCC4=O)cc3)CC2)c2ccccc21]

A suspension of 185 mg 4-chloro-1-methyl-2-oxo-1,2-dihydroquinoline-3-carbonitrile (845 μmol, CAS 150617-68-8, synthesis described in WO2012009649, example 1—compound Ill), 220 mg 1-{4-[(piperidin-4-yl)oxy]phenyl}pyrrolidin-2-one (845 μmol, intermediate 6) and 0.47 mL triethylamine (3.4 mmol) in 15 mL 2-propanol was stirred for 2 h at 90° C. and overnight at rt. After this time, water and ethyl acetate were added and the reaction was stirred for some time. The solid was collected by filtration to give 310 mg of the title compound (98% purity, 81% yield).

¹H NMR (DMSO-d₆) δ: 7.85-7.97 (m, 1H), 7.68-7.78 (m, 1H), 7.51-7.62 (m, 3H), 7.30-7.39 (m, 1H), 6.97-7.10 (m, 2H), 4.65-4.84 (m, 1H), 3.71-3.87 (m, 4H), 3.47-3.63 (m, 5H), 2.43-2.48 (m, 2H), 2.15-2.26 (m, 2H), 2.00-2.10 (m, 2H), 1.85-1.97 (m, 2H).

LC-MS (Method 2): Rₜ=1.08 min; MS (ESIpos): m/z=443.6 [M+H]⁺

### Example 19

### 4-{4-[4-(1H-imidazol-1-yl)phenoxy]piperidin-1-yl}-1-methyl-2-oxo-1,2-dihydroquinoline-3-carbonitrile

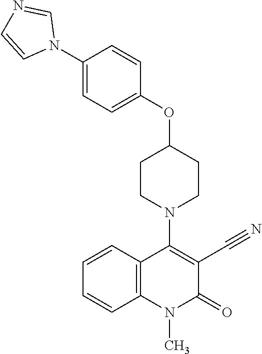 [SMILES: Cn1c(=O)c(C#N)c(N2CCC(Oc3ccc(-n4ccnc4)cc3)CC2)c2ccccc21]

A suspension of 45 mg 4-chloro-1-methyl-2-oxo-1,2-dihydroquinoline-3-carbonitrile (205 μmol, CAS 150617-68-8, synthesis described in WO2012009649, example 1—compound III), 50.0 mg 4-[4-(1H-imidazol-1-yl)phenoxy]piperidine (205 μmol, CAS 397277-13-3) and 0.11 mL triethylamine (820 μmol) in 8 mL 2-propanol was stirred for 4 h at 90° C. After this time, water and ethyl acetate were added and the reaction was stirred for some time. The solid was collected by filtration to give 57 mg of the title compound (90% purity, 59% yield).

¹H NMR (DMSO-d₆) δ: 8.12-8.17 (m, 1H), 7.82-7.90 (m, 1H), 7.71-7.77 (m, 1H), 7.64-7.67 (m, 1H), 7.53-7.60 (m, 3H), 7.32-7.39 (m, 1H), 7.15-7.25 (m, 2H), 7.08 (t, 1H), 4.76-4.91 (m, 1H), 3.72-3.84 (m, 2H), 3.53-3.65 (m, 5H), 2.18-2.28 (m, 2H), 1.88-2.01 (m, 2H).

LC-MS (Method 2): Rₜ=1.06 min; MS (ESIpos): m/z=426.8 [M+H]⁺

### Example 20

### 1-methyl-4-{4-[4-(morpholin-4-yl)phenoxy]piperidin-1-yl}-2-oxo-1,2-dihydroquinoline-3-carbonitrile

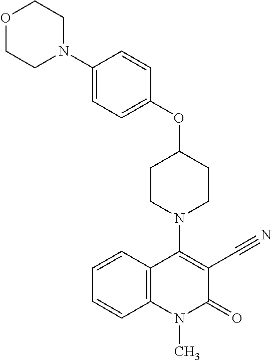 [SMILES: Cn1c(=O)c(C#N)c(N2CCC(Oc3ccc(N4CCOCC4)cc3)CC2)c2ccccc21]

A suspension of 8.3 mg 4-chloro-1-methyl-2-oxo-1,2-dihydroquinoline-3-carbonitrile (38.1 μmol, CAS 150617-68-8, synthesis described in WO2012009649, example 1—compound Ill), 10.0 mg 4-{4-[(piperidin-4-yl)oxy]phenyl}morpholine (38 μmol, intermediate 8) and 21 μL triethylamine (150 μmol) in 3 mL 2-propanol was stirred for 4 h at 90° C. After this time, water and ethyl acetate were added and the reaction was stirred for some time. The solid was collected by filtration to give 5 mg of the title compound (95% purity, 28% yield).

¹H NMR (DMSO-d₆) δ: 7.82-7.96 (m, 1H), 7.66-7.79 (m, 1H), 7.49-7.63 (m, 1H), 7.28-7.39 (m, 1H), 6.80-6.99 (m, 4H), 4.51-4.69 (m, 1H), 3.67-3.85 (m, 6H), 3.48-3.61 (m, 5H), 2.94-3.04 (m, 4H), 2.10-2.22 (m, 2H), 1.82-1.96 (m, 2H).

LC-MS (Method 2): Rₜ=1.17 min; MS (ESIpos): m/z=445.8 [M+H]⁺

### Example 21

### 1-methyl-4-{4-[4-(3-methyl-2-oxo-1,3-diazinan-1-yl)phenoxy]piperidin-1-yl}-2-oxo-1,2-dihydroquinoline-3-carbonitrile

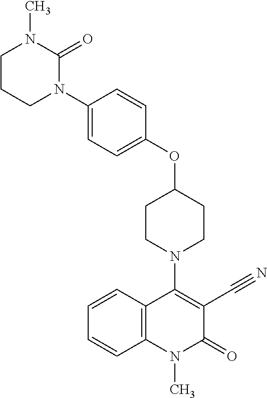 [SMILES: CN1CCCN(c2ccc(OC3CCN(c4c(C#N)c(=O)n(C)c5ccccc45)CC3)cc2)C1=O]

A suspension of 100 mg 4-[4-(4-bromophenoxy)piperidin-1-yl]-1-methyl-2-oxo-1,2-dihydroquinoline-3-carbonitrile (217 μmol, example 8), 50 mg 1-methyltetrahydropyrimidin-2(1H)-one (433 μmol, CAS 10166-54-8), 66 mg potassium carbonate (477 μmol), 8.3 mg copper(I)iodide (43 μmol) and 93 μL N,N′-dimethylethane-1,2-diamine (870 μmol) in 5 mL toluene was stirred for 30 h at 110° C. Water was added and the reaction was extracted with ethyl acetate (3×). The organic phase was washed with water and brine and dried over sodium sulfate. After evaporation of the solvent, the residue was purified by flash chromatography (silica, dichloromethane/methanol gradient 0-3%). 20 mg of the title compound were obtained (99% purity, 19% yield).

¹H NMR (DMSO-d₆) δ: 7.82-7.92 (m, 1H), 7.66-7.78 (m, 1H), 7.52-7.62 (m, 1H), 7.30-7.44 (m, 1H), 7.08-7.21 (m, 2H), 6.89-7.01 (m, 2H), 4.64-4.81 (m, 1H), 3.70-3.84 (m, 2H), 3.51-3.63 (m, 7H), 2.83 (s, 3H), 2.16-2.24 (m, 2H), 1.86-2.05 (m, 4H).

LC-MS (Method 2): Rₜ=1.08 min; MS (ESIpos): m/z=472.6 [M+H]⁺

### Example 22

### 1-methyl-4-{4-[4-(3-methyl-2-oxoimidazolidin-1-yl)phenoxy]piperidin-1-yl}-2-oxo-1,2-dihydroquinoline-3-carbonitrile

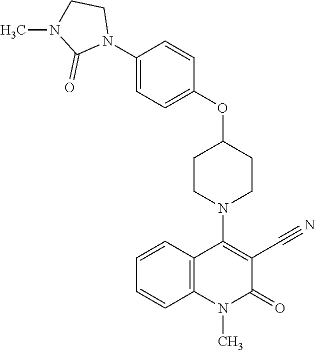 [SMILES: CN1CCN(c2ccc(OC3CCN(c4c(C#N)c(=O)n(C)c5ccccc45)CC3)cc2)C1=O]

A suspension of 80 mg 4-[4-(4-bromophenoxy)piperidin-1-yl]-1-methyl-2-oxo-1,2-dihydroquinoline-3-carbonitrile (173 μmol, example 8), 35 mg 1-methylimidazolidin-2-one (347 μmol, CAS 694-32-6), 53 mg potassium carbonate (381 μmol), 6.6 mg copper(I)iodide (35 μmol) and 75 μL N,N′-dimethylethane-1,2-diamine (690 μmol) in 5 mL toluene was stirred for 10 h at 110′C. Water was added and the reaction was extracted with ethyl acetate (3×). The organic phase was washed with water and brine and dried over sodium sulfate. After evaporation of the solvent, the residue was purified by flash chromatography (silica, dichloromethane/methanol gradient 0-3%). 5 mg of the title compound were obtained (91% purity, 6% yield).

¹H NMR (DMSO-d₆) δ: 7.83-7.96 (m, 1H), 7.67-7.77 (m, 1H), 7.52-7.60 (m, 1H), 7.41-7.51 (m, 2H), 7.28-7.37 (m, 1H), 6.95-7.08 (m, 2H), 4.62-4.73 (m, 1H), 3.69-3.83 (m, 4H), 3.49-3.62 (m, 5H), 3.36-3.46 (m, 2H), 2.74 (s, 3H), 2.10-2.26 (m, 2H), 1.82-1.97 (m, 2H).

LC-MS (Method 2): Rₜ=1.09 min; MS (ESIpos): m/z=458.6 [M+H]⁺

### Example 23

### 1-methyl-2-oxo-4-{4-[4-(2-oxopiperidin-1-yl)phenoxy]piperidin-1-yl}-1,2-dihydroquinoline-3-carbonitrile

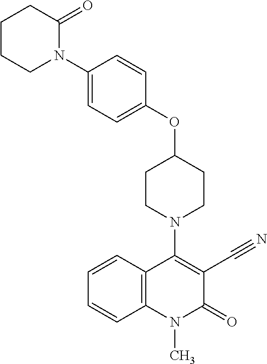 [SMILES: Cn1c(=O)c(C#N)c(N2CCC(Oc3ccc(N4CCCCC4=O)cc3)CC2)c2ccccc21]

A suspension of 80 mg 4-[4-(4-bromophenoxy)piperidin-1-yl]-1-methyl-2-oxo-1,2-dihydroquinoline-3-carbonitrile (173 μmol, example 8), 34 mg piperidin-2-one (347 μmol, CAS 675-20-7), 53 mg potassium carbonate (381 μmol), 6.6 mg copper(I)iodide (35 μmol) and 75 μL N,N′-dimethylethane-1,2-diamine (690 μmol) in 5 mL toluene was stirred for 10 h at 110° C.

Water was added and the reaction was extracted with ethyl acetate (3×). The organic phase was washed with water and brine and dried over sodium sulfate. After evaporation of the solvent, the residue was purified by flash chromatography (silica, dichloromethane/methanol gradient 0-3%). 5 mg of the title compound were obtained (91% purity, 6% yield).

¹H NMR (CHLOROFORM-d) δ: 7.78-7.87 (m, 1H), 7.61-7.70 (m, 1H), 7.34-7.42 (m, 1H), 7.25-7.27 (m, 1H), 7.15-7.22 (m, 2H), 6.92-7.03 (m, 2H), 4.56-4.73 (m, 1H), 3.83-3.98 (m, 2H), 3.69 (s, 3H), 3.56-3.66 (m, 4H), 2.49-2.64 (m, 2H), 2.20-2.33 (m, 2H), 2.04-2.15 (m, 2H), 1.87-2.02 (m, 4H).

LC-MS (Method 2): Rₜ=1.09 min; MS (ESIpos): m/z=457.6 [M+H]⁺

### Example 24

### 1-methyl-4-{4-[4-(4-methyl-2-oxopiperazin-1-yl)phenoxy]piperidin-1-yl}-2-oxo-1,2-dihydroquinoline-3-carbonitrile

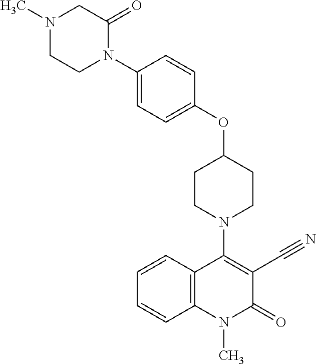 [SMILES: CN1CCN(c2ccc(OC3CCN(c4c(C#N)c(=O)n(C)c5ccccc45)CC3)cc2)C(=O)C1]

A suspension of 100 mg 4-[4-(4-bromophenoxy)piperidin-1-yl]-1-methyl-2-oxo-1,2-dihydroquinoline-3-carbonitrile (217 μmol, example 8), 50 mg 4-methylpiperazin-2-one (433 μmol, CAS 34770-60-0), 66 mg potassium carbonate (477 μmol), 8.3 mg copper(I)iodide (43 μmol) and 93 μL N,N′-dimethylethane-1,2-diamine (870 μmol) in 5 mL toluene was stirred for 30 h at 110′C. Water was added and the reaction was extracted with ethyl acetate. The organic phase was washed with water and brine and dried over sodium sulfate. After evaporation the residue was stirred in ethyl acetate, the solid was collected by filtration and dried in vacuum to give 26 mg of the title compound (24% yield, 95% purity).

¹H NMR (DMSO-d₆) δ: 7.84-7.97 (m, 1H), 7.69-7.79 (m, 1H), 7.52-7.63 (m, 1H), 7.29-7.38 (m, 1H), 7.14-7.26 (m, 2H), 7.01-7.09 (m, 2H), 4.70-4.82 (m, 1H), 3.70-3.85 (m, 2H), 3.50-3.65 (m, 7H), 3.02-3.13 (m, 2H), 2.68-2.74 (m, 2H), 2.28 (s, 3H), 2.15-2.25 (m, 2H), 1.86-2.00 (m, 2H).

LC-MS (Method 2): Rₜ=0.99 min; MS (ESIpos): m/z=472.8 [M+H]⁺

### Example 25

### 1-methyl-4-{4-[4-(3-methyl-2-oxo-2,3-dihydro-1H-imidazol-1-yl)phenoxy]piperidin-1-yl}-2-oxo-1,2-dihydroquinoline-3-carbonitrile

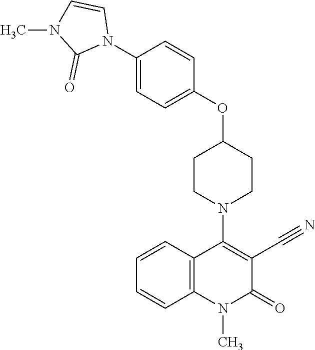 [SMILES: Cn1ccn(-c2ccc(OC3CCN(c4c(C#N)c(=O)n(C)c5ccccc45)CC3)cc2)c1=O]

Example 25 was obtained as a side product from the synthesis of example 22. Purification was performed by analogous chromatography and 11 mg were obtained (14% yield, 97% purity).

¹H NMR (CHLOROFORM-d) δ: 7.79-7.91 (m, 1H), 7.61-7.70 (m, 1H), 7.48-7.55 (m, 2H), 7.35-7.41 (m, 1H), 7.26 (br s, 1H), 6.97-7.05 (m, 2H), 6.47-6.55 (m, 1H), 6.28-6.33 (m, 1H), 4.65 (dt, 1H), 3.83-3.97 (m, 2H), 3.70 (s, 3H), 3.56-3.66 (m, 2H), 3.34 (s, 3H), 2.21-2.34 (m, 2H), 2.03-2.17 (m, 2H).

LC-MS (Method 2): Rₜ=1.04 min; MS (ESIpos): m/z=456.5 [M+H]⁺

### Example 26

### 4-{[1-(3-cyano-1-methyl-2-oxo-1,2-dihydroquinolin-4-yl)piperidin-4-yl]oxy}-N,N-dimethylbenzamide

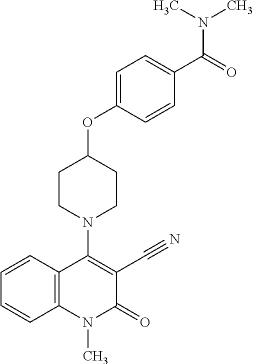 [SMILES: CN(C)C(=O)c1ccc(OC2CCN(c3c(C#N)c(=O)n(C)c4ccccc34)CC2)cc1]

To a suspension of 50.0 mg 4-(4-hydroxypiperidin-1-yl)-1-methyl-2-oxo-1,2-dihydroquinoline-3-carbonitrile (176 μmol, intermediate 1) and 118 mg 4-fluoro-N,N-dimethylbenzamide (706 μmol, CAS 24167-56-4) in 3 mL DMF at 0° C. was added 33 mg sodium hydride (60% in mineral oil, 829 μmol) and the mixture was stirred for 17 h at 80° C. After that time, water was added and the mixture was extracted with ethyl acetate (3×). The combined organic layers were filtered over a hydrophobic filter and concentrated under reduced pressure. The residue was purified by RP-HPLC (column: X-Bridge C18 5 μm 100×30 mm, mobile phase: acetonitrile/water (0.2 vol. % ammonia 32%)-gradient) to give 1.3 mg of the title compound (100% purity, 2% yield).

¹H NMR (DMSO-d₆) δ: 7.85-7.93 (m, 1H), 7.71-7.82 (m, 1H), 7.55-7.65 (m, 1H), 7.30-7.45 (m, 3H), 7.02-7.11 (m, 2H), 4.73-4.91 (m, 1H), 3.73-3.84 (m, 2H), 3.51-3.64 (m, 5H), 2.91-3.03 (m, 6H), 2.17-2.29 (m, 2H), 1.86-2.01 (m, 2H).

LC-MS (Method 2): Rₜ=1.04 min; MS (ESIpos): m/z=456.5 [M+H]⁺

### Example 27

### 4-{4-[(1,3-benzoxazol-4-yl)oxy]piperidin-1-yl}-1-methyl-2-oxo-1,2-dihydroquinoline-3-carbonitrile

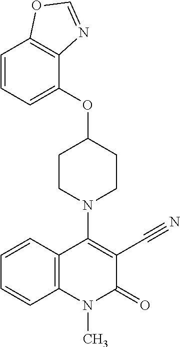 [SMILES: Cn1c(=O)c(C#N)c(N2CCC(Oc3cccc4ocnc34)CC2)c2ccccc21]

To a suspension of 100 mg 4-(4-hydroxypiperidin-1-yl)-1-methyl-2-oxo-1,2-dihydroquinoline-3-carbonitrile (353 μmol, intermediate 1), 91 mg 1,3-benzoxazol-4-ol (671 μmol, CAS 89590-22-7) and 185 mg triphenylphosphin (706 μmol) in 4 mL THF was added 140 μL diisopropyl azodicarboxylate (710 μmol) at 0° C. The mixture was stirred 48 h at rt. After that, the reaction was concentrated under reduced pressure. The residue was purified by flash chromatography (silica, hexane/ethyl acetate gradient 0-100%). The impure product was purified by RP-HPLC (column: X-Bridge C18 5 μm 100×30 mm, mobile phase: acetonitrile/water (0.1 vol. % formic acid)-gradient). The impure product was purified by preparative TLC (dichloromethane/ethanol; 95:5) to give 12 mg of the title compound (8% yield, 90% purity).

¹H NMR (DMSO-d₆) δ: 8.65-8.70 (m, 1H), 7.90 (d, 1H), 7.71-7.78 (m, 1H), 7.55-7.61 (m, 1H), 7.32-7.40 (m, 3H), 7.06-7.14 (m, 1H), 5.15-5.28 (m, 1H), 3.76-3.88 (m, 2H), 3.54-3.66 (m, 5H), 2.23-2.31 (m, 2H), 1.96-2.08 (m, 2H).

LC-MS (Method 1): Rₜ=1.17 min; MS (ESIpos): m/z=401 [M+H]⁺

### Example 28

### 1-methyl-2-oxo-4-(4-{[2-(trifluoromethyl)pyrimidin-5-yl]oxy}piperidin-1-yl)-1,2-dihydroquinoline-3-carbonitrile

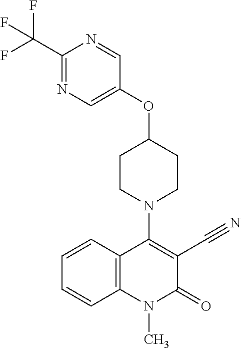 [SMILES: Cn1c(=O)c(C#N)c(N2CCC(Oc3cnc(C(F)(F)F)nc3)CC2)c2ccccc21]

A suspension of 292 mg 4-chloro-1-methyl-2-oxo-1,2-dihydroquinoline-3-carbonitrile (1.33 mmol, CAS 150617-68-8, synthesis described in WO2012009649, example 1—compound Ill), 330 mg 5-[(piperidin-4-yl)oxy]-2-(trifluoromethyl)pyrimidine (1.33 mmol, intermediate 10) and 740 μL triethylamine (5.3 mmol) in 15 mL 2-propanol was stirred for 2 h at 90° C. and 24 h at rt. After this time, water and ethyl acetate were added and the reaction was stirred for some time. The solid was collected by filtration to give 70 mg of the title compound (95% purity, 12% yield).

¹H NMR (DMSO-d₆) δ: 8.87-8.92 (m, 2H), 7.85-7.91 (m, 1H), 7.69-7.79 (m, 1H), 7.54-7.61 (m, 1H), 7.31-7.41 (m, 1H), 5.06-5.18 (m, 1H), 3.69-3.87 (m, 2H), 3.51-3.66 (m, 5H), 2.24-2.33 (m, 2H), 1.95-2.11 (m, 2H).

LC-MS (Method 2): Rₜ=1.19 min; MS (ESIpos): m/z=430.5 [M+H]⁺

### Example 29

### 4-{4-[(1,2-benzoxazol-6-yl)oxy]piperidin-1-yl}-1-methyl-2-oxo-1,2-dihydroquinoline-3-carbonitrile

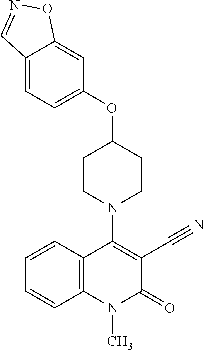 [SMILES: Cn1c(=O)c(C#N)c(N2CCC(Oc3ccc4cnoc4c3)CC2)c2ccccc21]

To a suspension of 100 mg 4-(4-hydroxypiperidin-1-yl)-1-methyl-2-oxo-1,2-dihydroquinoline-3-carbonitrile (353 μmol, intermediate 1), 91 mg 1,2-benzoxazol-6-ol (671 μmol, CAS 65685-55-4) and 185 mg triphenylphosphine (706 μmol) in 4 mL THF was added 140 μL diisopropyl azodicarboxylate (710 μmol). The mixture was stirred 48 h at rt. After that, the reaction was concentrated under reduced pressure. Water was added and the mixture was extracted with dichloromethane. The combined organic phases were washed with brine, filtered (using a waterresistant filter) and concentrated under reduced pressure. The residue was purified by RP-HPLC (column: X-Bridge C18 5 μm 100×30 mm, mobile phase: acetonitrile/water (0.2 vol. % ammonia 32%)-gradient). The impure product was stirred in ethanol, the solid was collected by filtration to give 41 mg of the title compound (23% yield, 80% purity).

¹H NMR (DMSO-d₆) δ: 10.63 (s, 1H), 7.84-7.94 (m, 1H), 7.74 (ddd, 1H), 7.49-7.63 (m, 2H), 7.29-7.40 (m, 1H), 6.46-6.76 (m, 2H), 4.75-4.92 (m, 1H), 3.70-3.84 (m, 2H), 3.51-3.67 (m, 5H), 2.15-2.31 (m, 2H), 1.87-2.02 (m, 2H).

LC-MS (Method 2): Rₜ=0.67 min; MS (ESIpos): m/z=401 [M+H]⁺

### Example 30

### 4-[4-(4-{[dimethyl(oxo)-λ⁶-sulfanylidene]amino}phenoxy)piperidin-1-yl]-1-methyl-2-oxo-1,2-dihydroquinoline-3-carbonitrile

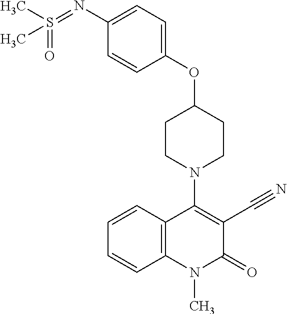 [SMILES: Cn1c(=O)c(C#N)c(N2CCC(Oc3ccc(N=S(C)(C)=O)cc3)CC2)c2ccccc21]

24 mg 1-methyl-2-oxo-4-(4-phenoxypiperidin-1-yl)-1,2-dihydroquinoline-3-carbonitrile (67 μmol, example 5), 37 mg (s-methanesulfonimidoyl)methane (401 μmol, CAS 1520-31-6), 5.7 mg 3,6-di-tert-butyl-10-phenyl-9-(2,4,6-trimethylphenyl)acridinium tetrafluoroborate (10 μmol) and 2.1 mg (2,2,6,6-tetramethylpiperidin-1-yl)oxidanyl (13 μmol) were dissolved in 800 μL 1,2-dichloroethane. The solution was degassed with argon for some time. The vial was placed in a water bath (to keep the temperature below 35,C) and was subsequently irradiated by two 40 W Kessil LED Aquarium lights (40 W each, 4 cm distance) for 24 h. The solvent was evaporated and the crude material was purified by RP-HPLC (column: Chromatorex 125×30 mm, 10 μm mobile phase: acetonitrile/water (0.2 vol. % ammonia 32%)-gradient) to give 3.5 mg of the title compound (11% yield, 93% purity).

¹H NMR (DMSO-d₆) δ: 7.85-7.91 (m, 1H), 7.69-7.81 (m, 1H), 7.53-7.62 (m, 1H), 7.30-7.40 (m, 1H), 6.82-6.93 (m, 4H), 4.55-4.67 (m, 1H), 3.71-3.83 (m, 2H), 3.46-3.61 (m, 5H), 3.10-3.19 (m, 6H), 2.12-2.25 (m, 2H), 1.83-1.94 (m, 2H).

LC-MS (Method 2): Rₜ=0.98 min; MS (ESIpos): m/z=451.5 [M+H]⁺

### Example 31

### 4-[4-(2-methoxy-4-methylphenoxy)piperidin-1-yl]-1-methyl-2-oxo-1,2-dihydroquinoline-3-carbonitrile

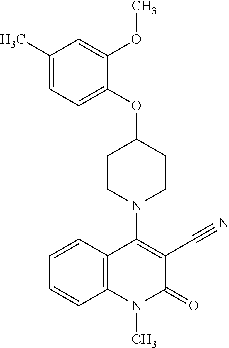 [SMILES: COc1cc(C)ccc1OC1CCN(c2c(C#N)c(=O)n(C)c3ccccc23)CC1]

A solution of 100 mg 4-chloro-1-methyl-2-oxo-1,2-dihydroquinoline-3-carbonitrile (434 μmol, CAS 150617-68-8, synthesis described in WO2012009649, example 1—compound III), 115 mg 4-(2-methoxy-4-methylphenoxy)piperidine (521 μmol, CAS 883543-21-3) and 0.12 mL triethylamine (0.87 mmol) in 7.5 mL 2-propanol was stirred for 4 h at 90° C. After this time, water was added and the reaction was extracted with ethyl acetate. The organic phase was washed with water and brine and dried over sodium sulfate. After evaporation of the solvent, the residue was purified by RP-HPLC (column: X-Bridge C18 5 μm 100×30 mm, mobile phase: acetonitrile/water (0.2 vol. % ammonia 32%)-gradient). 68 mg of the title compound were obtained (95% purity, 37% yield).

¹H NMR (DMSO-d₆) δ: 7.81-7.91 (m, 1H), 7.70-7.79 (m, 1H), 7.52-7.62 (m, 1H), 7.30-7.38 (m, 1H), 6.90-7.01 (m, 1H), 6.75-6.87 (m, 1H), 6.64-6.72 (m, 1H), 4.47-4.62 (m, 1H), 3.73-3.83 (m, 5H), 3.55-3.59 (m, 3H), 3.45-3.55 (m, 2H), 2.25 (s, 3H), 2.13 (ddd, 2H), 1.82-1.95 (m, 2H).

LC-MS (Method 2): Rₜ=1.29 min; MS (ESIpos): m/z=404.5 [M+H]⁺

### Example 32

### 4-[4-(4-cyanophenoxy)piperidin-1-yl]-1-methyl-2-oxo-1,2-dihydroquinoline-3-carbonitrile

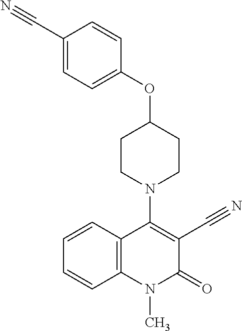 [SMILES: Cn1c(=O)c(C#N)c(N2CCC(Oc3ccc(C#N)cc3)CC2)c2ccccc21]

A solution of 100 mg 4-chloro-1-methyl-2-oxo-1,2-dihydroquinoline-3-carbonitrile (457 μmol, CAS 150617-68-8, synthesis described in WO2012009649, example 1—compound III), 111 mg 4-[(piperidin-4-yl)oxy]benzonitrile (549 μmol, CAS 224178-67-0) and 0.24 mL N,N-diisopropylethylamine (1.4 mmol) in 2 mL 2-propanol was stirred for 2 h at 90° C. After this time, water was added and the reaction was stirred for some time. The residue was collected by filtration and washed with ethanol. The resulting solid was dried at 100° C. in vacuum to give 159 mg of the title compound (86% yield, 95% purity).

¹H NMR (DMSO-d₆) δ: 7.86-7.89 (m, 1H), 7.71-7.81 (m, 3H), 7.55-7.62 (m, 1H), 7.29-7.39 (m, 1H), 7.19-7.27 (m, 2H), 4.87-4.99 (m, 1H), 3.70-3.84 (m, 2H), 3.53-3.64 (m, 5H), 2.18-2.28 (m, 2H), 1.89-2.02 (m, 2H).

LC-MS (Method 1): Rₜ=1.19 min; MS (ESIpos): m/z=385 [M+H]⁺

### Example 33

### 4-[4-(2-fluorophenoxy)piperidin-1-yl]-1-methyl-2-oxo-1,2-dihydroquinoline-3-carbonitrile

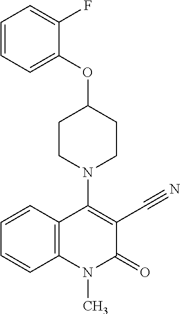 [SMILES: Cn1c(=O)c(C#N)c(N2CCC(Oc3ccccc3F)CC2)c2ccccc21]

A solution of 100 mg 4-chloro-1-methyl-2-oxo-1,2-dihydroquinoline-3-carbonitrile (457 μmol, CAS 150617-68-8, synthesis described in WO2012009649, example 1—compound III), 107 mg 4-(2-fluorophenoxy)piperidine (549 μmol, CAS 3623-02-7) and 0.24 mL N,N-diisopropylethylamine (1.4 mmol) in 2 mL 2-propanol was stirred for 2 h at 90° C. After this time, water was added and the reaction was stirred for some time. The residue was collected by filtration and washed with ethanol. The resulting solid was dried at 100° C. in vacuum to give 145 mg of the title compound (75% yield, 90% purity).

¹H NMR (DMSO-d₆) δ: 7.80-7.93 (m, 1H), 7.66-7.78 (m, 1H), 7.52-7.59 (m, 1H), 7.07-7.40 (m, 4H), 6.92-7.03 (m, 1H), 4.71-4.85 (m, 1H), 3.73-3.85 (m, 2H), 3.47-3.63 (m, 5H), 2.17-2.27 (m, 2H), 1.86-2.07 (m, 2H).

LC-MS (Method 1): Rₜ=1.28 min; MS (ESIpos): m/z=378 [M+H]⁺

### Example 34

### 4-[4-(2-cyanophenoxy)piperidin-1-yl]-1-methyl-2-oxo-1,2-dihydroquinoline-3-carbonitrile

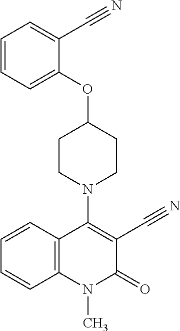 [SMILES: Cn1c(=O)c(C#N)c(N2CCC(Oc3ccccc3C#N)CC2)c2ccccc21]

A solution of 100 mg 4-chloro-1-methyl-2-oxo-1,2-dihydroquinoline-3-carbonitrile (457 μmol, CAS 150617-68-8, synthesis described in WO2012009649, example 1—compound III), 111 mg 2-[(piperidin-4-yl)oxy]benzonitrile (549 μmol, CAS 900572-37-4) and 0.24 mL N,N-diisopropylethylamine (1.4 mmol) in 2 mL 2-propanol was stirred for 2 h at 90° C. After this time, water was added and the reaction was stirred for some time. The residue was collected by filtration and washed with ethanol. The resulting solid was dried at 100° C. in vacuum to give 163 mg of the title compound (88% yield, 95% purity).

¹H NMR (DMSO-d₆) δ: 7.86-7.93 (m, 1H), 7.64-7.79 (m, 3H), 7.55-7.60 (m, 1H), 7.41-7.48 (m, 1H), 7.32-7.39 (m, 1H), 7.06-7.18 (m, 1H), 4.94-5.05 (m, 1H), 3.72-3.85 (m, 2H), 3.53-3.68 (m, 5H), 2.20-2.31 (m, 2H), 1.90-2.06 (m, 2H).

LC-MS (Method 1): Rₜ=1.18 min; MS (ESIpos): m/z=385 [M+H]⁺

### Example 35

### 4-{[1-(3-cyano-1-methyl-2-oxo-1,2-dihydroquinolin-4-yl)piperidin-4-yl]oxy}benzamide

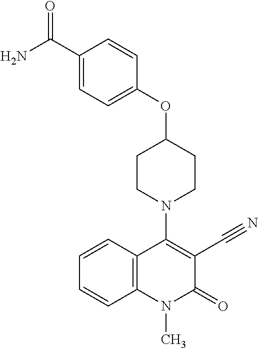 [SMILES: Cn1c(=O)c(C#N)c(N2CCC(Oc3ccc(C(N)=O)cc3)CC2)c2ccccc21]

A solution of 100 mg 4-chloro-1-methyl-2-oxo-1,2-dihydroquinoline-3-carbonitrile (457 μmol, CAS 150617-68-8, synthesis described in WO2012009649, example 1—compound III), 121 mg 4-[(piperidin-4-yl)oxy]benzamide (549 μmol, CAS 609781-30-8) and 0.24 mL N,N-diisopropylethylamine (1.4 mmol) in 2 mL 2-propanol was stirred for 2 h at 90° C. After this time, water was added and the reaction was stirred for some time. The residue was collected by filtration and washed with ethanol. The resulting solid was dried at 100° C. in vacuum to give 166 mg of the title compound (86% yield, 95% purity).

¹H NMR (DMSO-d₆) δ: 7.81-7.92 (m, 4H), 7.71-7.78 (m, 1H), 7.55-7.62 (m, 1H), 7.30-7.38 (m, 1H), 7.16-7.25 (m, 1H), 7.03-7.11 (m, 2H), 4.80-4.92 (m, 1H), 3.70-3.84 (m, 2H), 3.51-3.65 (m, 5H), 2.16-2.28 (m, 2H), 1.86-2.01 (m, 2H).

LC-MS (Method 1): Rₜ=0.95 min; MS (ESIpos): m/z=403 [M+H]⁺

### Example 36

### 1-methyl-2-oxo-4-{4-[2-(trifluoromethyl)phenoxy]piperidin-1-yl}-1,2-dihydroquinoline-3-carbonitrile

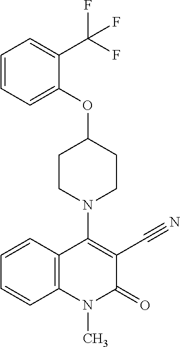 [SMILES: Cn1c(=O)c(C#N)c(N2CCC(Oc3ccccc3C(F)(F)F)CC2)c2ccccc21]

A solution of 100 mg 4-chloro-1-methyl-2-oxo-1,2-dihydroquinoline-3-carbonitrile (457 μmol, CAS 150617-68-8, synthesis described in WO2012009649, example 1—compound III), 135 mg 4-[2-(trifluoromethyl)phenoxy]piperidine (549 μmol, CAS 824390-04-7) and 0.24 mL N,N-diisopropylethylamine (1.4 mmol) in 2 mL 2-propanol was stirred for 2 h at 90° C. After this time, water was added and the reaction was stirred for some time. The residue was collected by filtration and washed with ethanol. The resulting solid was dried at 100° C. in vacuum to give 166 mg of the title compound (87% yield, 95% purity).

¹H NMR (DMSO-d₆) δ: 7.82-7.90 (m, 1H), 7.68-7.78 (m, 1H), 7.51-7.67 (m, 3H), 7.28-7.49 (m, 2H), 7.02-7.17 (m, 1H), 4.90-5.11 (m, 1H), 3.67-3.81 (m, 2H), 3.48-3.65 (m, 5H), 2.12-2.28 (m, 2H), 1.87-2.06 (m, 2H).

LC-MS (Method 1): Rₜ=1.37 min; MS (ESIpos): m/z=428 [M+H]⁺

### Example 37

### 4-[4-(4-cyclopropylphenoxy)piperidin-1-yl]-1-methyl-2-oxo-1,2-dihydroquinoline-3-carbonitrile

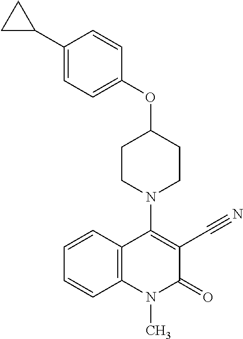 [SMILES: Cn1c(=O)c(C#N)c(N2CCC(Oc3ccc(C4CC4)cc3)CC2)c2ccccc21]

30 mg 4-[4-(4-bromophenoxy)piperidin-1-yl]-1-methyl-2-oxo-1,2-dihydroquinoline-3-carbonitrile (68 μmol, example 8) and 6.0 mg di-μ-iodobis(tri-tert-butylphosphino)dipalladium(l) (7.0 μmol, CAS 166445-62-1) were sealed in a vessel and flushed with argon. 1.1 mL toluene was added and the mixture was stirred at rt. 410 μL bromo(cyclopropyl)zinc (0.50 M, 210 μmol, CAS 126403-68-7) was added dropwise. The mixture was stirred at rt for 1 h. The mixture was filtered via a silica column. The column was washed with dichloromethane and dichloromethane/methanol (9:1). The filtrate was concentrated under reduced pressure. The residue was purified by RP-HPLC (instrument: Waters Autopurificationsystem; column: X-Bridge C18 5μ 50×50 mm; eluent A: water (0.1 vol. % formic acid 99%), eluent B: acetonitrile; gradient: 0.00-0.50 min. 36% B (40->100 mL/min), 0.51-13 min. 36-56% B (100 mL/min), DAD scan: 210-400 nm) to give 4.1 mg of the title compound (88% purity, 13% yield).

¹H NMR (400 MHz, ACETONITRILE-d₃) δ ppm 0.52-0.64 (m, 2H); 0.78-0.97 (m, 3H); 1.22-1.30 (m, 4H); 1.80-1.89 (m, 1H); 3.49-3.61 (m+s, 7H); 3.75-3.84 (m, 2H); 4.63 (tt, 1H); 6.85-6.92 (m, 2H); 6.99-7.07 (m, 2H); 7.25-7.31 (m, 1H); 7.44-7.50 (m, 1H); 7.67 (ddd, 1H); 7.89 (dd, 1H).

### Example 38

### 1-methyl-2-oxo-4-{4-[3-(trifluoromethoxy)phenoxy]piperidin-1-yl}-1,2-dihydroquinoline-3-carbonitrile

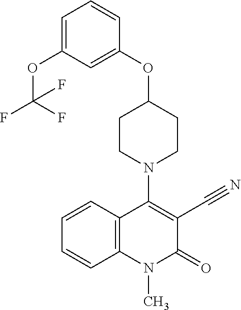 [SMILES: Cn1c(=O)c(C#N)c(N2CCC(Oc3cccc(OC(F)(F)F)c3)CC2)c2ccccc21]

A solution of 100 mg 4-chloro-1-methyl-2-oxo-1,2-dihydroquinoline-3-carbonitrile (457 μmol, CAS 150617-68-8, synthesis described in WO2012009649, example 1—compound III), 131 mg 4-[3-(trifluoromethoxy)phenoxy]piperidine (503 μmol, CAS 459819-38-6) and 0.13 mL triethylamine (910 μmol) in 6.3 mL 2-propanol was stirred for 4 h at 90° C. After this time, water was added and the reaction was extracted with ethyl acetate. The organic phase was washed with water and brine and dried over sodium sulfate. After evaporation of the solvent, the residue was purified by flash chromatography (silica, dichloromethane/methanol gradient 0-1%). 120 mg of the title compound were obtained (95% purity, 56% yield).

¹H NMR (DMSO-d₆) δ: 7.82-7.92 (m, 1H), 7.70-7.80 (m, 1H), 7.53-7.62 (m, 1H), 7.41-7.47 (m, 1H), 7.30-7.39 (m, 1H), 7.04-7.15 (m, 2H), 6.90-7.01 (m, 1H), 4.78-4.91 (m, 1H), 3.72-3.82 (m, 2H), 3.51-3.63 (m, 5H), 2.16-2.27 (m, 2H), 1.84-2.01 (m, 2H).

LC-MS (Method 2): Rₜ=1.40 min; MS (ESIpos): m/z=444.5 [M+H]⁺

### Example 39

### 4-{4-[4-(difluoromethoxy)phenoxy]piperidin-1-yl}-1-methyl-2-oxo-1,2-dihydroquinoline-3-carbonitrile

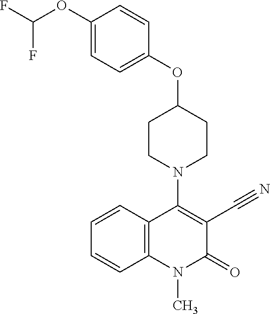 [SMILES: Cn1c(=O)c(C#N)c(N2CCC(Oc3ccc(OC(F)F)cc3)CC2)c2ccccc21]

A suspension of 100 mg 4-chloro-1-methyl-2-oxo-1,2-dihydroquinoline-3-carbonitrile (457 μmol, CAS 150617-68-8, synthesis described in WO2012009649, example 1—compound Ill), 73 mg 4-[4-(difluoromethoxy)phenoxy]piperidine hydrogen chloride salt (1:1) (261 μmol, intermediate 12) and 0.11 mL N,N-diisopropylethylamine (650 μmol) in 1.3 mL 2-propanol was stirred for 2 h at 90° C. After this time, water was added and the reaction was stirred for some time. The residue was collected by filtration and washed with water and ethanol. The resulting solid was dried to give 88 mg of the title compound (95% yield, 100% purity).

¹H NMR (DMSO-d₆) δ: 7.82-7.95 (m, 1H), 7.70-7.80 (m, 1H), 7.52-7.62 (m, 1H), 6.87-7.45 (m, 6H), 4.65-4.82 (m, 1H), 3.70-3.84 (m, 2H), 3.48-3.63 (m, 5H), 2.11-2.26 (m, 2H), 1.85-1.98 (m, 2H).

LC-MS (Method 1): Rₜ=1.29 min; MS (ESIpos): m/z=426.4 [M+H]⁺

### Example 40

### 1-methyl-4-[4-methyl-4-(4-nitrophenoxy)piperidin-1-yl]-2-oxo-1,2-dihydroquinoline-3-carbonitrile

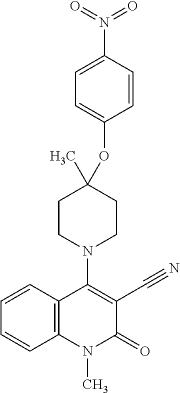 [SMILES: Cn1c(=O)c(C#N)c(N2CCC(C)(Oc3ccc([N+](=O)[O-])cc3)CC2)c2ccccc21]

A solution of 239 mg 4-chloro-1-methyl-2-oxo-1,2-dihydroquinoline-3-carbonitrile (1.04 mmol, CAS 150617-68-8, synthesis described in WO2012009649, example 1—compound III), 270 mg 4-methyl-4-(4-nitrophenoxy)piperidine (1.14 mmol, intermediate 14) and 0.29 mL triethylamine (2.1 mmol) in 14 mL 2-propanol was stirred for 5 h at 90° C. After this time, water was added and the reaction was extracted with ethyl acetate. The organic phase was washed with water and brine and dried over sodium sulfate. After evaporation of the solvent, DMSO was added and the reaction was stirred for some time. The residue was collected by filtration to give 125 mg of the title compound (95% purity, 27% yield).

¹H NMR (DMSO-d₆) δ: 8.14-8.26 (m, 2H), 7.86-7.94 (m, 1H), 7.70-7.78 (m, 1H), 7.53-7.60 (m, 1H), 7.28-7.40 (m, 3H), 3.52-3.75 (m, 7H), 2.20-2.30 (m, 2H), 2.04-2.15 (m, 2H), 1.56 (s, 3H).

LC-MS (Method 2): Rₜ=1.27 min; MS (ESIpos): m/z=419.4 [M+H]⁺

### Example 41

### 4-[4-(4-aminophenoxy)-4-methylpiperidin-1-yl]-1-methyl-2-oxo-1,2-dihydroquinoline-3-carbonitrile

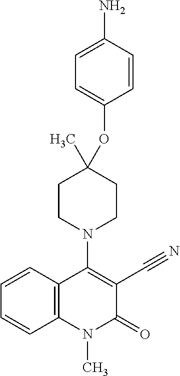 [SMILES: Cn1c(=O)c(C#N)c(N2CCC(C)(Oc3ccc(N)cc3)CC2)c2ccccc21]

100 mg 1-methyl-4-[4-methyl-4-(4-nitrophenoxy)piperidin-1-yl]-2-oxo-1,2-dihydroquinoline-3-carbonitrile (227 μmol, example 40) and 10 mg palladium (94.0 μmol, 10% on activated car) were solved in 10 mL methanol and 10 mL dichloromethane. The mixture was stirred at rt under hydrogen atmosphere for 6 h. The reaction was filtered via kieselgur. After evaporation of the solvent 96 mg of the title compound were obtained (95% purity, 103% yield).

¹H NMR (DMSO-d₆) δ: 7.85-7.95 (m, 1H), 7.70-7.79 (m, 1H), 7.60 (br d, J=1.0 Hz, 1H), 7.29-7.38 (m, 1H), 6.47-6.87 (m, 4H), 3.74-3.92 (m, 2H), 3.50-3.64 (m, 5H), 1.82-2.03 (m, 4H), 1.19-1.27 (m, 3H).

LC-MS (Method 2): Rₜ=1.08 min; MS (ESIpos): m/z=389.4 [M+H]⁺

### Example 42

### 4-[4-(4-bromophenoxy)-4-methylpiperidin-1-yl]-1-methyl-2-oxo-1,2-dihydroquinoline-3-carbonitrile

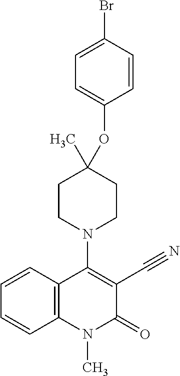 [SMILES: Cn1c(=O)c(C#N)c(N2CCC(C)(Oc3ccc(Br)cc3)CC2)c2ccccc21]

To a mixture of 80 mg 4-[4-(4-aminophenoxy)-4-methylpiperidin-1-yl]-1-methyl-2-oxo-1,2-dihydroquinoline-3-carbonitrile (206 μmol, example 41) in 630 μL tribromomethane (7.2 mmol) at 100° C. was added dropwise a solution of 55 μL 3-m ethylbutyl nitrite (410 μmol) in 180 μL tribromomethane (2.1 mmol). The reaction was stirred for 4 h at 100° C. The mixture was concentrated under reduced pressure and purified by flash chromatography (silica, dichloromethane/methanol gradient 0-1%) to give 33 mg of the title compound (95% purity, 34% yield).

¹H NMR (DMSO-d₆) δ: 7.86-7.92 (m, 1H), 7.71-7.77 (m, 1H), 7.42-7.63 (m, 3H), 7.28-7.37 (m, 1H), 7.00-7.15 (m, 2H), 3.50-3.89 (m, 8H), 1.85-2.17 (m, 6H).

LC-MS (Method 2): Rₜ=1.40 min; MS (ESIpos): m/z=453.3 [M+H]⁺

### Example 43

### 4-{4-[4-(methanesulfonyl)phenoxy]piperidin-1-yl}-1-methyl-2-oxo-1,2-dihydroquinoline-3-carbonitrile

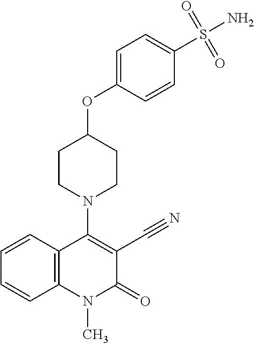 [SMILES: Cn1c(=O)c(C#N)c(N2CCC(Oc3ccc(S(N)(=O)=O)cc3)CC2)c2ccccc21]

A suspension of 13 mg 4-chloro-1-methyl-2-oxo-1,2-dihydroquinoline-3-carbonitrile (58.7 μmol, CAS 150617-68-8, synthesis described in WO2012009649, example 1—compound III), 18 mg 4-[4-(methanesulfonyl)phenoxy]piperidine (70.5 μmol, intermediate 16) and 41 μL N,N-diisopropylethylamine (230 μmol) in 250 μL 2-propanol was stirred for 4 h at rt. The mixture was concentrated under reduced pressure. The residue was solved in dichloromethane and concentrated under reduced pressure again. The residue was purified by RP-HPLC (column: Chromatorex 125×30 mm, 10 μm mobile phase: acetonitrile/water (0.2 vol. % ammonia 32%)-gradient) to give 23 mg of the title compound (99% purity, 89% yield).

¹H NMR (DMSO-d₆) δ: 7.80-7.92 (m, 3H), 7.69-7.78 (m, 1H), 7.52-7.61 (m, 1H), 7.22-7.39 (m, 3H), 4.88-5.00 (m, 1H), 3.70-3.85 (m, 2H), 3.50-3.66 (m, 5H), 3.17 (s, 3H), 2.18-2.31 (m, 2H), 1.87-2.04 (m, 2H).

LC-MS (Method 1): Rₜ=1.06 min; MS (ESIpos): m/z=438.4 [M+H]⁺

### Example 44

### 4-{4-[4-(2-methoxyethoxy)phenoxy]piperidin-1-yl}-1-methyl-2-oxo-1,2-dihydroquinoline-3-carbonitrile

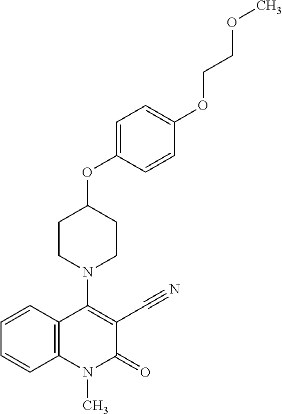 [SMILES: COCCOc1ccc(OC2CCN(c3c(C#N)c(=O)n(C)c4ccccc34)CC2)cc1]

A suspension of 51 mg 4-chloro-1-methyl-2-oxo-1,2-dihydroquinoline-3-carbonitrile (232 μmol, CAS 150617-68-8, synthesis described in WO2012009649, example 1—compound III), 70 mg 4-[4-(2-methoxyethoxy)phenoxy]piperidine (279 μmol, intermediate 21) and 160 μL N,N-diisopropylethylamine (930 μmol) in 1 mL 2-propanol was stirred for 2 h at rt. The mixture was concentrated under reduced pressure. The residue was purified by RP-HPLC (column: Chromatorex 125×30 mm, 10 μm mobile phase: acetonitrile/water-gradient) to give 29 mg of the title compound (99% purity, 29% yield).

¹H NMR (DMSO-d₆) δ: 7.80-7.91 (m, 1H), 7.68-7.77 (m, 1H), 7.57 (dd, 1H), 7.34 (td, 1H), 6.93-7.01 (m, 2H), 6.84-6.91 (m, 2H), 4.53-4.70 (m, 1H), 3.98-4.07 (m, 2H), 3.70-3.81 (m, 2H), 3.61-3.66 (m, 2H), 3.48-3.60 (m, 5H), 3.28 (s, 3H), 2.17 (ddd, 2H), 1.83-1.95 (m, 2H).

LC-MS (Method 1): Rₜ=1.20 min; MS (ESIpos): m/z=434.8 [M+H]⁺

### Example 45

### 4-{4-[4-(dimethylamino)phenoxy]piperidin-1-yl}-1-methyl-2-oxo-1,2-dihydroquinoline-3-carbonitrile

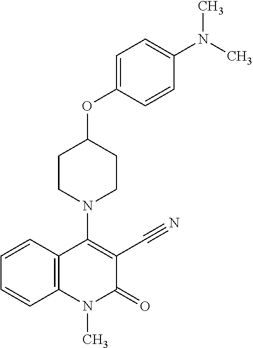 [SMILES: CN(C)c1ccc(OC2CCN(c3c(C#N)c(=O)n(C)c4ccccc34)CC2)cc1]

A suspension of 66 mg 4-chloro-1-methyl-2-oxo-1,2-dihydroquinoline-3-carbonitrile (303 μmol, CAS 150617-68-8, synthesis described in WO2012009649, example 1—compound III), 80 mg N,N-dimethyl-4-[(piperidin-4-yl)oxy]aniline (363 μmol, intermediate 22) and 0.21 mL N,N-diisopropylethylamine (1.2 mmol) in 1.3 mL 2-propanol was stirred for 3 h at rt. After this time, water was added and the reaction was extracted with ethyl acetate (3×). The combined organic phases were filtered (using a waterresistant filter) and concentrated under reduced pressure. The residue was purified by RP-HPLC (column: X-Bridge C18 5 μm 100×30 mm, mobile phase: acetonitrile/water (0.2 vol. % ammonia 32%)-gradient). 25 mg of the title compound were obtained (98% purity, 20% yield).

¹H NMR (DMSO-d₆) δ: 7.82-7.94 (m, 1H), 7.66-7.78 (m, 1H), 7.52-7.63 (m, 1H), 7.29-7.38 (m, 1H), 6.86-6.98 (m, 2H), 6.67-6.77 (m, 2H), 4.49-4.62 (m, 1H), 3.70-3.87 (m, 2H), 3.46-3.62 (m, 5H), 2.81 (s, 6H), 2.09-2.22 (m, 2H), 1.82-1.97 (m, 2H).

LC-MS (Method 2): Rₜ=1.27 min; MS (ESIpos): m/z=403.3 [M+H]⁺

### Example 46

### 4-[4-(4-methoxyphenoxy)piperidin-1-yl]-1-methyl-2-oxo-1,2-dihydroquinoline-3-carbonitrile

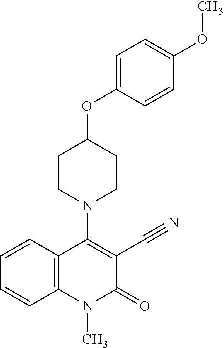 [SMILES: COc1ccc(OC2CCN(c3c(C#N)c(=O)n(C)c4ccccc34)CC2)cc1]

A suspension of 44 mg 4-chloro-1-methyl-2-oxo-1,2-dihydroquinoline-3-carbonitrile (201 μmol, CAS 150617-68-8, synthesis described in WO2012009649, example 1—compound III), 50 mg 4-(4-methoxyphenoxy)piperidine (241 μmol, intermediate 23) and 0.14 mL N,N-diisopropylethylamine (800 μmol) in 870 μL 2-propanol was stirred for 2 h at rt. After this time, water was added and the reaction was extracted with ethyl acetate (2×). The combined organic phases were filtered (using a waterresistant filter) and concentrated under reduced pressure. The residue was purified by RP-HPLC (column: X-Bridge C18 5 μm 100×30 mm, mobile phase: acetonitrile/water-gradient). 12 mg of the title compound were obtained (97% purity, 15% yield).

¹H NMR (DMSO-d₆) δ: 7.82-7.93 (m, 1H), 7.68-7.76 (m, 1H), 7.53-7.62 (m, 1H), 7.31-7.39 (m, 1H), 6.82-7.02 (m, 4H), 4.54-4.68 (m, 1H), 3.66-3.83 (m, 5H), 3.46-3.60 (m, 5H), 2.12-2.25 (m, 2H), 1.79-1.97 (m, 2H).

LC-MS (Method 1): Rₜ=1.24 min; MS (ESIpos): m/z=390.6 [M+H]⁺

### Example 47

### 1-methyl-2-oxo-4-(4-{4-[(propan-2-yl)oxy]phenoxy}piperidin-1-yl)-1,2-dihydroquinoline-3-carbonitrile

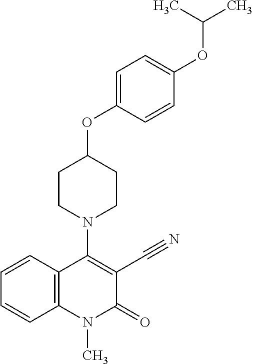 [SMILES: CC(C)Oc1ccc(OC2CCN(c3c(C#N)c(=O)n(C)c4ccccc34)CC2)cc1]

A solution of 100 mg 4-chloro-1-methyl-2-oxo-1,2-dihydroquinoline-3-carbonitrile (457 μmol, CAS 150617-68-8, synthesis described in WO2012009649, example 1—compound III), 129 mg 4-{4-[(propan-2-yl)oxy]phenoxy}piperidine (549 μmol, intermediate 24) and 320 μL N,N-diisopropylethylamine (1.8 mmol) in 2 mL 2-propanol was stirred for 4 h at rt. After this time, water (50 ml) was added and the reaction was stirred for 1 h. The solid was collected by filtration, washed with water and dried under reduced pressure at 60° C. The residue was purified by RP-HPLC (column: X-Bridge C18 5 μm 100×30 mm, mobile phase: acetonitrile/water (0.2 vol. % ammonia 32%)-gradient). 38.2 mg of the title compound were obtained (99% purity, 20% yield).

1H NMR (400 MHz, DMSO-d6) δ ppm 7.87 (dd, 1H), 7.74 (ddd, 1H), 7.57 (dd, 1H), 7.34 (td, 1H), 6.91-7.00 (m, 2H), 6.82-6.90 (m, 2H), 4.56-4.67 (m, 1H), 4.48 (dt, 1H), 3.70-3.82 (m, 2H), 3.48-3.61 (m, 5H), 2.17 (ddd, 2H), 1.81-1.98 (m, 2H), 1.23 (d, 6H).

LC-MS (Method 1): Rₜ=1.34 min; MS (ESIpos): m/z=418.3 [M+H]⁺

### Example 48

### 1-methyl-2-oxo-4-{4-[4-(trifluoromethoxy)phenoxy]piperidin-1-yl}-1,2-dihydroquinoline-3-carboxamide

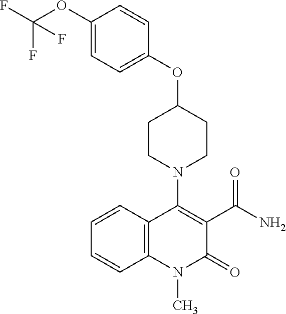 [SMILES: Cn1c(=O)c(C(N)=O)c(N2CCC(Oc3ccc(OC(F)(F)F)cc3)CC2)c2ccccc21]

A mixture of 580 mg 1-methyl-2-oxo-4-{4-[4-(trifluoromethoxy)phenoxy]piperidin-1-yl}-1,2-dihydroquinoline-3-carbonitrile (1.24 mmol, example 2), 70 mg palladium(II)diacetate (311 μmol) and 734 mg N-[(1E)-ethylidene]hydroxylamine (12.4 mmol, CAS 107-29-9) in 9.3 mL ethanol was stirred for 9 h at 80° C. Water was added and the reaction was extracted with ethyl acetate (2×). The organic phase was washed with brine and dried over sodium sulfate. After evaporation the residue was purified by flash chromatography (silica, dichloromethane/methanol gradient 0-3%) to give 530 mg of the title compound (95% purity, 88% yield).

¹H NMR (DMSO-d₆) δ: 7.83-8.03 (m, 1H), 7.59-7.74 (m, 2H), 7.40-7.58 (m, 2H), 7.24-7.36 (m, 3H), 7.06-7.19 (m, 2H), 4.58-4.70 (m, 1H), 3.59 (s, 3H), 3.34-3.45 (m, 2H), 3.07-3.22 (m, 2H), 2.09-2.20 (m, 2H), 1.78-1.93 (m, 2H).

LC-MS (Method 2): Rₜ=1.27 min; MS (ESIpos): m/z=462.5 [M+H]⁺

### Example 49

### 4-[4-(3-chlorophenoxy)piperidin-1-yl]-1-methyl-2-oxo-1,2-dihydroquinoline-3-carboxamide

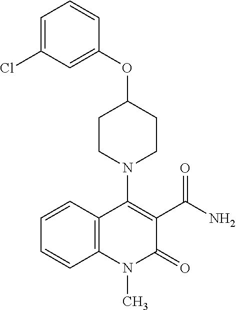 [SMILES: Cn1c(=O)c(C(N)=O)c(N2CCC(Oc3cccc(Cl)c3)CC2)c2ccccc21]

A mixture of 50 mg 4-[4-(3-chlorophenoxy)piperidin-1-yl]-1-methyl-2-oxo-1,2-dihydroquinoline-3-carbonitrile (121 μmol, example 9), 7 mg palladium(II)diacetate (30 μmol) and 71 mg N-[(1E)-ethylidene]hydroxylamine (1.2 mmol, CAS 107-29-9) in 5 mL ethanol was stirred for 6 h at 80° C. Water was added and the reaction was extracted with ethyl acetate (2×). The organic phase was washed with brine and dried over sodium sulfate. After evaporation the residue was purified by RP-HPLC (column: X-Bridge C18 5 μm 100×30 mm, mobile phase: acetonitrile/water (0.2 vol. % ammonia 32%)-gradient) to give 20 mg of the title compound (95% purity, 38% yield).

¹H NMR (400 MHz, DMSO-d₆) δ ppm 1.79-1.90 (m, 2H); 2.07-2.18 (m, 2H); 3.11-3.21 (m, 2H); 3.34-3.42 (m, 2H); 3.58 (s, 3H); 4.67 (dt, 1H); 6.97-7.01 (m, 2H); 7.10-7.12 (m, 1H); 7.28-7.33 (m, 2H); 7.53 (s, 2H); 7.59-7.65 (m, 1H); 7.68 (s, 1H); 7.91 (dd, 1H).

### Example 50

### 4-[4-(4-chlorophenoxy)piperidin-1-yl]-1-methyl-2-oxo-1,2-dihydroquinoline-3-carboxamide

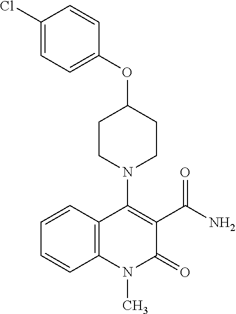 [SMILES: Cn1c(=O)c(C(N)=O)c(N2CCC(Oc3ccc(Cl)cc3)CC2)c2ccccc21]

A mixture of 50 mg 4-[4-(4-chlorophenoxy)piperidin-1-yl]-1-methyl-2-oxo-1,2-dihydroquinoline-3-carbonitrile (121 μmol, example 10), 7 mg palladium(II)diacetate (30 μmol) and 71 mg N-[(1E)-ethylidene]hydroxylamine (1.2 mmol, CAS 107-29-9) in 5 mL ethanol was stirred for 4 h at 80° C. Water was added and the reaction was extracted with ethyl acetate (2×). The organic phase was washed with brine and dried over sodium sulfate. After evaporation the residue was purified by flash chromatography (silica, dichloromethane/methanol gradient 0-3%) to give 30 mg of the title compound (95% purity, 57% yield).

¹H NMR (DMSO-d₆) δ: 7.85-7.99 (m, 1H), 7.57-7.73 (m, 2H), 7.43-7.57 (m, 2H), 7.25-7.38 (m, 3H), 7.00-7.09 (m, 2H), 4.53-4.70 (m, 1H), 3.50-3.65 (m, 3H), 3.34-3.41 (m, 2H), 3.09-3.22 (m, 2H), 2.06-2.19 (m, 2H), 1.76-1.89 (m, 2H).

LC-MS (Method 2): Rₜ=1.20 min; MS (ESIpos): m/z=412.5 [M+H]⁺

### Example 51

### ethyl 4-{[1-(3-carbamoyl-1-methyl-2-oxo-1,2-dihydroquinolin-4-yl)piperidin-4-yl]oxy}benzoate

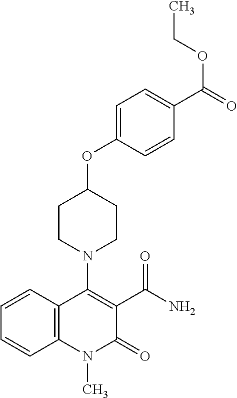 [SMILES: CCOC(=O)c1ccc(OC2CCN(c3c(C(N)=O)c(=O)n(C)c4ccccc34)CC2)cc1]

To a solution of 56 mg ethyl 4-{[1-(3-cyano-1-methyl-2-oxo-1,2-dihydroquinolin-4-yl)piperidin-4-yl]oxy}benzoate (130 μmol, example 12) in 2.0 mL ethanol were added 39 mg N-[(1E)-ethylidene]hydroxylamine (651 μmol, CAS 107-29-9) and 7 mg palladium(II)diacetate (33 μmol). The mixture was stirred for 3 h at 80° C. Water was added and the reaction was extracted with ethyl acetate (3×). The combined organic layers were filtered and concentrated under reduced pressure. The residue was purified by RP-HPLC (column: X-Bridge C18 5 μm 100×30 mm, mobile phase: acetonitrile/water (0.2 vol. % ammonia 32%)-gradient) to give 36.1 mg of the title compound (100% purity, 62% yield). 1H-NMR (400 MHz, DMSO-d6): 5 [ppm]=1.30 (t, 3H), 1.79-2.00 (m, 2H), 2.10-2.22 (m, 2H), 3.11-3.24 (m, 2H), 3.34-3.46 (m, 2H), 3.58 (s, 3H), 4.27 (q, 2H), 4.73 (dt, 1H), 7.08-7.15 (m, 2H), 7.27-7.35 (m, 1H), 7.48 (d, 1H), 7.50-7.54 (m, 1H), 7.59-7.66 (m, 1H), 7.68 (br d, 1H), 7.85-7.94 (m, 3H).

LC-MS (Method 3): Rₜ=1.22 min; MS (ESIpos): m/z=450 [M+H]⁺

### Example 52

### 4-{4-[4-(difluoromethoxy)phenoxy]piperidin-1-yl}-1-methyl-2-oxo-1,2-dihydroquinoline-3-carboxamide

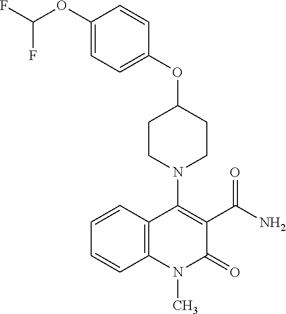 [SMILES: Cn1c(=O)c(C(N)=O)c(N2CCC(Oc3ccc(OC(F)F)cc3)CC2)c2ccccc21]

A mixture of 50 mg 4-{4-[4-(difluoromethoxy)phenoxy]piperidin-1-yl}-1-methyl-2-oxo-1,2-dihydroquinoline-3-carbonitrile (118 μmol, example 39), 7 mg palladium(II)diacetate (29 μmol) and 70 mg N-[(1E)-ethylidene]hydroxylamine (1.2 mmol, CAS 107-29-9) in 1 mL ethanol was stirred for 6 h at 80° C. Water was added and the re action was extracted with ethyl acetate (2×). The organic phase was washed with brine and dried over sodium sulfate. After evaporation the residue was purified by RP-HPLC (column: Chromatorex 125×30 mm, 10 μm mobile phase: acetonitrile/water (0.2 vol. % ammonia 32%)-gradient) to give 28.8 mg of the title compound (100% purity, 55% yield).

¹H NMR (DMSO-d₆) δ: 7.91 (dd, 1H); 7.68 (s, 1H); 7.59-7.65 (m, 1H); 7.52 (d, 1H); 7.47 (br d, 1H); 6.90-7.35 (m, 6H); 4.53-4.62 (m, 1H); 3.58 (s, 3H); 3.36-3.42 (m, 2H); 3.09-3.19 (m, 2H); 2.07-2.17 (m, 2H); 1.79-1.90 (m, 2H).

LC-MS (Method 2): Rₜ=1.15 min; MS (ESIpos): m/z=444.4 [M+H]⁺

### Example 53

### 1-methyl-2-oxo-4-[4-(phenylsulfanyl)piperidin-1-yl]-1,2-dihydroquinoline-3-carbonitrile

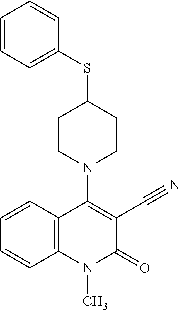 [SMILES: Cn1c(=O)c(C#N)c(N2CCC(Sc3ccccc3)CC2)c2ccccc21]

A suspension of 100 mg 4-chloro-1-methyl-2-oxo-1,2-dihydroquinoline-3-carbonitrile (457 μmol, CAS 150617-68-8, synthesis described in WO2012009649, example 1—compound Ill), 126 mg 4-(phenylsulfanyl)piperidine hydrogen chloride salt (1:1) (549 μmol, CAS 101798-66-7) and 0.24 mL N,N-diisopropylethylamine (1.4 mmol) in 2.0 mL 2-propanol was stirred for 2 h at 90° C. After this time, water was added and the reaction was stirred for some time. The residue was collected by filtration and washed with ethanol. The resulting solid was dried to give 141 mg of the title compound (78% yield, 95% purity).

¹H NMR (DMSO-d₆) δ: 7.80-7.85 (m, 1H), 7.68-7.76 (m, 1H), 7.53-7.61 (m, 1H), 7.42-7.49 (m, 2H), 7.24-7.41 (m, 4H), 3.72-3.82 (m, 2H), 3.62-3.72 (m, 1H), 3.54-3.61 (m, 3H), 3.42-3.53 (m, 2H), 2.05-2.18 (m, 2H), 1.71-1.86 (m, 2H).

LC-MS (Method 1): Rₜ=1.35 min; MS (ESIpos): m/z=376 [M+H]⁺

### Example 54

### 7-bromo-1-methyl-2-oxo-4-(4-phenoxypiperidin-1-yl)-1,2-dihydroquinoline-3-carbonitrile

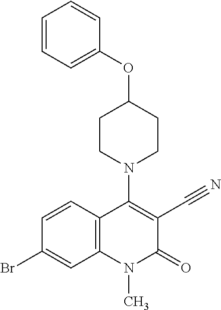 [SMILES: Cn1c(=O)c(C#N)c(N2CCC(Oc3ccccc3)CC2)c2ccc(Br)cc21]

A suspension of 100 mg 7-bromo-4-chloro-1-methyl-2-oxo-1,2-dihydroquinoline-3-carbonitrile (336 μmol, intermediate 27), 71.5 mg 4-phenoxypiperidine (403 μmol, CAS 3202-33-3) and 0.18 mL N,N-diisopropylethylamine (1.0 mmol) in 2.0 mL 2-propanol was stirred for 2 h at 90° C. After this time, water was added and the reaction was stirred for some time. The residue was collected by filtration and washed with ethanol. The resulting solid was dried to give 123 mg of the title compound (79% yield, 95% purity).

¹H NMR (DMSO-d₆) δ: 7.72-7.83 (m, 2H), 7.47-7.51 (m, 1H), 7.26-7.35 (m, 2H), 7.02-7.09 (m, 2H), 6.89-6.98 (m, 1H), 4.68-4.88 (m, 1H), 3.70-3.81 (m, 2H), 3.49-3.61 (m, 5H), 2.12-2.26 (m, 2H), 1.85-1.99 (m, 2H).

LC-MS (Method 1): Rₜ=1.40 min; MS (ESIpos): m/z=438 [M+H]⁺

### Example 55

### 7-bromo-1-methyl-2-oxo-4-[4-(phenylsulfanyl)piperidin-1-yl]-1,2-dihydroquinoline-3-carbonitrile

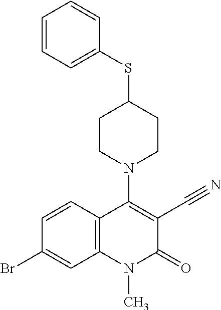 [SMILES: Cn1c(=O)c(C#N)c(N2CCC(Sc3ccccc3)CC2)c2ccc(Br)cc21]

A suspension of 100 mg 7-bromo-4-chloro-1-methyl-2-oxo-1,2-dihydroquinoline-3-carbonitrile (336 μmol, intermediate 27), 93 mg 4-(phenylsulfanyl)piperidine hydrogen chloride salt (1:1) (403 μmol, CAS 101798-66-7) and 0.18 mL N,N-diisopropylethylamine (1.0 mmol) in 2.0 mL 2-propanol was stirred for 2 h at 90° C. After this ti me, water was added and the reaction was stirred for some time. The residue was collected by filtration and washed with ethanol. The resulting solid was dried to give 141 mg of the title compound (87% yield, 95% purity).

¹H NMR (DMSO-d₆) δ: 7.67-7.80 (m, 2H), 7.43-7.51 (m, 3H), 7.32-7.40 (m, 2H), 7.23-7.32 (m, 1H), 3.63-3.81 (m, 3H), 3.41-3.58 (m, 5H), 2.04-2.17 (m, 2H), 1.73-1.86 (m, 2H).

LC-MS (Method 1): Rₜ=1.46 min; MS (ESIpos): m/z=454 [M+H]⁺

### Example 56

### 7-bromo-4-[4-(4-fluorophenoxy)piperidin-1-yl]-1-methyl-2-oxo-1,2-dihydroquinoline-3-carbonitrile

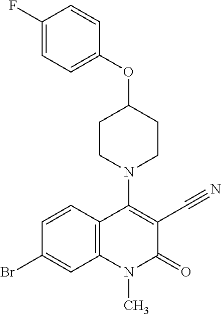 [SMILES: Cn1c(=O)c(C#N)c(N2CCC(Oc3ccc(F)cc3)CC2)c2ccc(Br)cc21]

A suspension of 64 mg 7-bromo-4-chloro-1-methyl-2-oxo-1,2-dihydroquinoline-3-carbonitrile (213 μmol, intermediate 27), 50 mg 4-(4-fluorophenoxy)piperidine hydrogen chloride salt (256 μmol, CAS 3202-34-4) and 0.11 mL N,N-diisopropylethylamine (640 μmol) in 1.3 mL 2-propanol was stirred for 2 h at 90° C. After this ti me, water was added and the reaction was stirred for some time. The residue was collected by filtration and washed with ethanol. The resulting solid was dried to give 80 mg of the title compound (78% yield, 95% purity).

¹H NMR (DMSO-d₆) δ: 7.72-7.82 (m, 2H), 7.49 (dd, J=8.9, 1.8 Hz, 1H), 7.01-7.20 (m, 4H), 4.62-4.78 (m, 1H), 3.69-3.80 (m, 2H), 3.47-3.61 (m, 5H), 2.07-2.23 (m, 2H), 1.78-2.02 (m, 2H).

LC-MS (Method 1): Rₜ=1.40 min; MS (ESIpos): m/z=458 [M+H]⁺

### Example 57

### 7-bromo-4-[4-(4-chlorophenoxy)piperidin-1-yl]-1-methyl-2-oxo-1,2-dihydroquinoline-3-carbonitrile

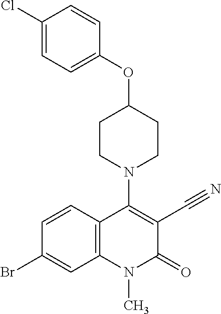 [SMILES: Cn1c(=O)c(C#N)c(N2CCC(Oc3ccc(Cl)cc3)CC2)c2ccc(Br)cc21]

A suspension of 200 mg 7-bromo-4-chloro-1-methyl-2-oxo-1,2-dihydroquinoline-3-carbonitrile (672 μmol, intermediate 27), 157 mg 4-(4-chlorophenoxy)piperidine (739 μmol, CAS 97839-99-1) and 190 μL triethylamine (1.3 mmol) in 9.3 mL 2-propanol was stirred for 5 h at 90° C. After this time, water was added and the reaction was extracted with ethyl acetate. The organic phase was washed with water and brine and dried over sodium sulfate. After evaporation of the solvent, ethyl acetate was added and the reaction was stirred for some time. The residue was collected by filtration to give 116 mg of the title compound (95% purity, 35% yield).

¹H NMR (DMSO-d₆) δ: 7.69-7.84 (m, 2H), 7.43-7.55 (m, 1H), 7.27-7.38 (m, 2H), 7.01-7.11 (m, 2H), 4.69-4.83 (m, 1H), 3.67-3.81 (m, 2H), 3.47-3.62 (m, 5H), 2.11-2.26 (m, 2H), 1.82-1.98 (m, 2H).

LC-MS (Method 2): Rₜ=1.48 min; MS (ESIpos): m/z=472 [M+H]⁺

### Example 58

### 7-bromo-1-methyl-2-oxo-4-{4-[4-(trifluoromethoxy)phenoxy]piperidin-1-yl}-1,2-dihydroquinoline-3-carbonitrile

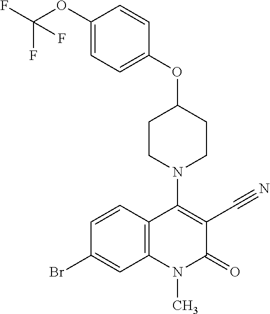 [SMILES: Cn1c(=O)c(C#N)c(N2CCC(Oc3ccc(OC(F)(F)F)cc3)CC2)c2ccc(Br)cc21]

A solution of 150 mg 7-bromo-4-chloro-1-methyl-2-oxo-1,2-dihydroquinoline-3-carbonitrile (504 μmol, intermediate 27), 145 mg 4-[4-(trifluoromethoxy)phenoxy]piperidine (555 μmol, CAS 287952-67-4) and 0.14 mL triethylamine (1.0 mmol) in 2-propanol was stirred for 3 h at 90° C. After this time, water was added and the reaction was extracted with ethyl acetate. The organic phase was washed with water and brine and dried over sodium sulfate. After evaporation of the solvent, the residue was purified by flash chromatography (silica, dichloromethane/methanol gradient 0-1%). 175 mg of the title compound were obtained (95% purity, 63% yield).

¹H NMR (DMSO-d₆) δ: 7.72-7.85 (m, 2H), 7.45-7.54 (m, 1H), 7.27-7.33 (m, 2H), 7.09-7.21 (m, 2H), 4.78 (tt, 1H), 3.67-3.83 (m, 2H), 3.47-3.63 (m, 5H), 2.13-2.26 (m, 2H), 1.84-1.99 (m, 2H).

LC-MS (Method 2): Rₜ=1.50 min; MS (ESIpos): m/z=523 [M+H]⁺

### Example 59

### 7-bromo-1-methyl-2-oxo-4-{4-[4-(trifluoromethyl)phenoxy]piperidin-1-yl}-1,2-dihydroquinoline-3-carbonitrile

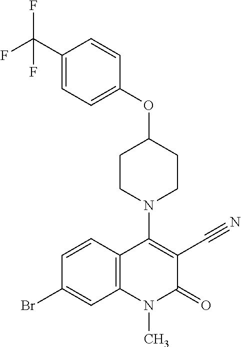 [SMILES: Cn1c(=O)c(C#N)c(N2CCC(Oc3ccc(C(F)(F)F)cc3)CC2)c2ccc(Br)cc21]

A suspension of 174 mg 7-bromo-4-chloro-1-methyl-2-oxo-1,2-dihydroquinoline-3-carbonitrile (584 μmol, intermediate 27), 172 mg 4-[4-(trifluoromethyl)phenoxy]piperidine (701 μmol, CAS 28033-37-6) and 0.31 mL N,N-diisopropylethylamine (1.8 mmol) in 3.5 mL 2-propanol was stirred for 2 h at 90° C. After this time, water was added and the reaction was stirred for some time. The residue was collected by filtration and washed with ethanol. The resulting solid was dried to give 267 mg of the title compound (86% yield, 95% purity).

¹H NMR (DMSO-d₆) δ: 7.73-7.82 (m, 2H), 7.63-7.73 (m, 2H), 7.44-7.55 (m, 1H), 7.19-7.30 (m, 2H), 4.83-5.00 (m, 1H), 3.69-3.82 (m, 2H), 3.50-3.62 (m, 5H), 2.16-2.28 (m, 2H), 1.87-2.00 (m, 2H).

LC-MS (Method 1): Rₜ=1.49 min; MS (ESIpos): m/z=508 [M+H]⁺

### Example 60

### 7-bromo-4-[4-(3-chlorophenoxy)piperidin-1-yl]-1-methyl-2-oxo-1,2-dihydroquinoline-3-carbonitrile

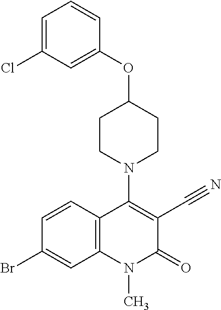 [SMILES: Cn1c(=O)c(C#N)c(N2CCC(Oc3cccc(Cl)c3)CC2)c2ccc(Br)cc21]

A solution of 200 mg 7-bromo-4-chloro-1-methyl-2-oxo-1,2-dihydroquinoline-3-carbonitrile (672 μmol, intermediate 27), 157 mg 4-(3-chlorophenoxy)piperidine (739 μmol, CAS 97840-40-9) and 0.19 mL triethylamine (1.3 mmol) in 9.3 mL 2-propanol was stirred for 5 h at 90° C. After this time, water was added and the reaction was extracted with ethyl acetate. The organic phase was washed with water and brine and dried over sodium sulfate. After evaporation of the solvent, the residue was purified by flash chromatography (silica, dichloromethane/methanol gradient 0-1%). 250 mg of the title compound were obtained (95% purity, 75% yield).

¹H NMR (DMSO-d₆) δ: 7.70-7.84 (m, 2H), 7.45-7.54 (m, 1H), 7.28-7.39 (m, 1H), 7.12-7.19 (m, 1H), 6.98-7.06 (m, 2H), 4.78-4.95 (m, 1H), 3.69-3.81 (m, 2H), 3.49-3.64 (m, 5H), 2.13-2.26 (m, 2H), 1.83-1.97 (m, 2H).

LC-MS (Method 2): Rₜ=1.47 min; MS (ESIpos): m/z=472 [M+H]⁺

### Example 61

### 7-bromo-1-methyl-2-oxo-4-{4-[3-(trifluoromethoxy)phenoxy]piperidin-1-yl}-1,2-dihydroquinoline-3-carbonitrile

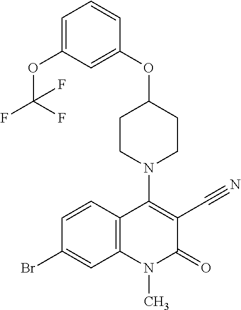 [SMILES: Cn1c(=O)c(C#N)c(N2CCC(Oc3cccc(OC(F)(F)F)c3)CC2)c2ccc(Br)cc21]

A solution of 300 mg 7-bromo-4-chloro-1-methyl-2-oxo-1,2-dihydroquinoline-3-carbonitrile (1.01 mmol, intermediate 27), 290 mg 4-[3-(trifluoromethoxy)phenoxy]piperidine (1.11 mmol, CAS 459819-38-6) and 0.28 mL triethylamine (2.0 mmol) in 14 mL 2-propanol was stirred for 5 h at 90° C. After this time, water was added and the reaction was extracted with ethyl acetate. The organic phase was washed with water and brine and dried over sodium sulfate. After evaporation of the solvent, the residue was purified by flash chromatography (silica, dichloromethane/methanol gradient 0-1%). 370 mg of the title compound were obtained (95% purity, 76% yield).

¹H NMR (DMSO-d₆) δ: 7.73-7.82 (m, 2H), 7.37-7.52 (m, 2H), 7.05-7.15 (m, 2H), 6.94 (dt, 1H), 4.78-4.90 (m, 1H), 3.68-3.81 (m, 2H), 3.48-3.61 (m, 5H), 2.15-2.25 (m, 2H), 1.80-2.00 (m, 2H).

LC-MS (Method 2): Rₜ=1.49 min; MS (ESIpos): m/z=523 [M+H]⁺

### Example 62

### 7-hydroxy-1-methyl-2-oxo-4-{4-[4-(trifluoromethoxy)phenoxy]piperidin-1-yl}-1,2-dihydroquinoline-3-carbonitrile

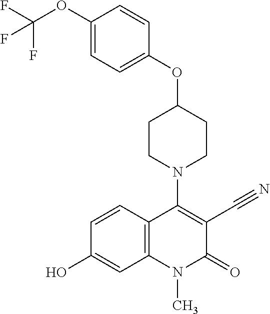 [SMILES: Cn1c(=O)c(C#N)c(N2CCC(Oc3ccc(OC(F)(F)F)cc3)CC2)c2ccc(O)cc21]

A suspension of 100 mg 4-chloro-7-hydroxy-1-methyl-2-oxo-1,2-dihydroquinoline-3-carbonitrile (426 μmol, intermediate 31), 111 mg 4-[4-(trifluoromethoxy)phenoxy]piperidine (426 μmol, CAS 287952-67-4) and 120 μL triethylamine (850 μmol) in 5 mL 2-propanol was stirred for 2 h at 90° C. After this time, water and ethyl acetate were added and the reaction was stirred for some time. The solid was collected by filtration. The impure product was stirred in dichloromethane/methanol for some time. The residue was collected by filtration to give 100 mg of the title compound (95% purity, 49% yield).

¹H NMR (DMSO-d₆) δ: 10.80 (br s, 1H), 7.67-7.75 (m, 1H), 7.24-7.40 (m, 2H), 7.06-7.23 (m, 2H), 6.70-6.85 (m, 2H), 4.69-4.83 (m, 1H), 3.64-3.81 (m, 2H), 3.46-3.56 (m, 5H), 2.12-2.24 (m, 2H), 1.80-1.95 (m, 2H).

LC-MS (Method 1): Rₜ=1.27 min; MS (ESIpos): m/z=460 [M+H]⁺

### Example 63

### 7-fluoro-1-methyl-2-oxo-4-{4-[4-(trifluoromethoxy)phenoxy]piperidin-1-yl}-1,2-dihydroquinoline-3-carbonitrile

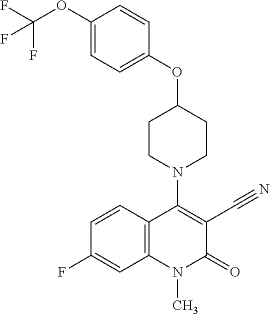 [SMILES: Cn1c(=O)c(C#N)c(N2CCC(Oc3ccc(OC(F)(F)F)cc3)CC2)c2ccc(F)cc21]

A suspension of 120 mg 4-chloro-7-fluoro-1-methyl-2-oxo-1,2-dihydroquinoline-3-carbonitrile (487 μmol, intermediate 34), 157 mg 4-[4-(trifluoromethoxy)phenoxy]piperidine (584 μmol, CAS 287952-67-4) and 250 μL triethylamine (1.5 mmol) in 3 mL 2-propanol was stirred for 4 h at 90° C. After this time, water was added and the reaction was stirred for some time. The solid was collected by filtration, washed with water, ethanol and hexane and dried. 216 mg of the title compound was obtained (99% purity, 95% yield).

¹H NMR (DMSO-d₆) δ: 7.93 (dd, 1H), 7.46 (dd, 1H), 7.28-7.34 (m, 2H), 7.12-7.23 (m, 3H), 4.78 (dt, 1H), 3.70-3.80 (m, 2H), 3.50-3.60 (m, 5H), 2.20 (ddd, 2H), 1.86-1.97 (m, 2H).

LC-MS (Method 1): Rₜ=1.44 min; MS (ESIpos): m/z=462 [M+H]⁺

### Example 64

### 7-chloro-1-methyl-2-oxo-4-{4-[4-(trifluoromethoxy)phenoxy]piperidin-1-yl}-1,2-dihydroquinoline-3-carbonitrile

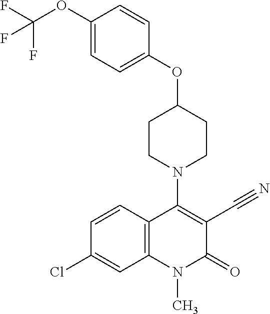 [SMILES: Cn1c(=O)c(C#N)c(N2CCC(Oc3ccc(OC(F)(F)F)cc3)CC2)c2ccc(Cl)cc21]

A suspension of 120 mg 4,7-dichloro-1-methyl-2-oxo-1,2-dihydroquinoline-3-carbonitrile (450 μmol, intermediate 37), 164 mg 4-[4-(trifluoromethoxy)phenoxy]piperidine (541 μmol, CAS 287952-67-4) and 240 μL triethylamine (1.4 mmol) in 3 mL 2-propanol was stirred for 2 h at 90° C. After this time, water was added and the reaction was stirred for some time. The solid was collected by filtration, washed with water and hexane and dried. 198 mg of the title compound was obtained (99% purity, 83% yield).

¹H NMR (DMSO-d₆) δ: 7.86 (d, 1H), 7.66 (d, 1H), 7.37 (dd, 1H), 7.27-7.34 (m, 2H), 7.11-7.17 (m, 2H), 4.78 (dt, 1H), 3.71-3.80 (m, 2H), 3.49-3.60 (m, 5H), 2.16-2.25 (m, 2H), 1.86-1.97 (m, 2H).

LC-MS (Method 2): Rₜ=1.49 min; MS (ESIpos): m/z=478 [M+H]⁺

### Example 65

### 4-[4-(4-chlorophenoxy)piperidin-1-yl]-7-methoxy-1-methyl-2-oxo-1,2-dihydroquinoline-3-carbonitrile

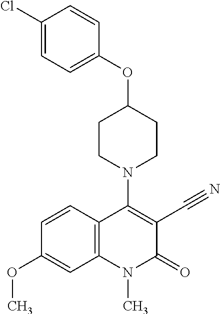 [SMILES: COc1ccc2c(N3CCC(Oc4ccc(Cl)cc4)CC3)c(C#N)c(=O)n(C)c2c1]

30 mg 7-bromo-4-[4-(4-chlorophenoxy)piperidin-1-yl]-1-methyl-2-oxo-1,2-dihydroquinoline-3-carbonitrile (64 μmol, example 57), 5.5 mg (2′-amino[biphenyl]-2-yl)(methanesulfonato-kappaO)palladium-di-tert-butyl(2′,4′,6′-triisopropyl-3,6-dimethoxy [biphenyl]-2-yl)phosphine (1:1) (6.4 μmol, CAS 1536473-72-9), 3.1 mg di-tert-butyl[3,6-dimethoxy-2′,4′,6′-tri(propan-2-yl)biphenyl-2-yl]phosphine (6.4 μmol, CAS 1160861-53-9) and 29 mg cesium carbonate (89 μmol) were sealed in a vessel and flushed with argon. 1.0 mL toluene and 26 μL methanol were added and the mixture was stirred overnight at 60° C. The mixture was filtered via a silica column. The column was washed with dichloromethane/methanol (9:1). The filtrate was concentrated under reduced pressure. The residue was purified by RP-HPLC (column: X-Bridge C18 5 μm 100×30 mm, mobile phase: acetonitrile/water (0.2 vol. % ammonia 32%)-gradient) to give 2.4 mg of the title compound (95% purity, 8% yield).

¹H NMR (ACETONITRILE-d₃) δ: 7.59-7.70 (m, 1H), 7.07-7.15 (m, 2H), 6.79-6.87 (m, 2H), 6.66-6.76 (m, 2H), 4.44-4.56 (m, 1H), 3.76 (s, 3H), 3.55-3.66 (m, 2H), 3.33-3.44 (m, 5H), 2.00-2.09 (m, 2H), 1.79-1.84 (m, 2H).

### Example 66

### 7-methoxy-1-methyl-2-oxo-4-{4-[4-(trifluoromethyl)phenoxy]piperidin-1-yl}-1,2-dihydroquinoline-3-carbonitrile

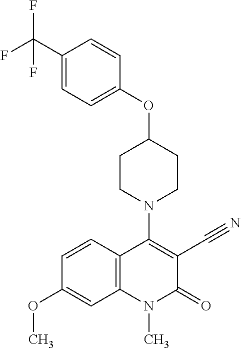 [SMILES: COc1ccc2c(N3CCC(Oc4ccc(C(F)(F)F)cc4)CC3)c(C#N)c(=O)n(C)c2c1]

mg 7-bromo-1-methyl-2-oxo-4-{4-[4-(trifluoromethyl)phenoxy]piperidin-1-yl}-1,2-dihydro-quinoline-3-carbonitrile (59 μmol, example 59), 5.1 mg (2′-amino[biphenyl]-2-yl)(methanesulfonato-kappaO)palladium-di-tert-butyl(2′,4′,6′-triisopropyl-3,6-dimethoxy [biphenyl]-2-yl)phosphine (1:1) (5.9 μmol, CAS 1536473-72-9), 2.9 mg di-tert-butyl[3,6-dimethoxy-2′,4′,6′-tri(propan-2-yl)biphenyl-2-yl]phosphine (5.9 μmol, CAS 1160861-53-9) and 27 mg cesium carbonate (83 μmol) were sealed in a vessel and flushed with argon. 1.0 mL toluene and 24 μL methanol were added and the mixture was stirred overnight at 60° C. The mixture was filtered via a silica column. The column was washed with dichloromethane/methanol (9:1). The filtrate was concentrated under reduced pressure. The residue was purified by RP-HPLC (column: X-Bridge C18 5 μm 100×30 mm, mobile phase: acetonitrile/water (0.2 vol. % ammonia 32%)-gradient) to give 3.6 mg of the title compound (100% purity, 13% yield).

¹H NMR (ACETONITRILE-d₃) δ: 7.60 (d, 1H), 7.41 (d, 2H), 6.93 (d, 2H), 6.61-6.71 (m, 2H), 4.53-4.65 (m, 1H), 3.72 (s, 3H), 3.53-3.64 (m, 2H), 3.29-3.43 (m, 5H), 2.03 (ddt, Hz, 2H), 1.74-1.82 (m, 2H).

### Example 67

### 1,7-dimethyl-2-oxo-4-{4-[4-(trifluoromethoxy)phenoxy]piperidin-1-yl}-1,2-dihydroquinoline-3-carbonitrile

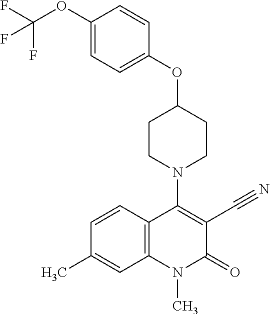 [SMILES: Cc1ccc2c(N3CCC(Oc4ccc(OC(F)(F)F)cc4)CC3)c(C#N)c(=O)n(C)c2c1]

A suspension of 122 mg 4-chloro-1,7-dimethyl-2-oxo-1,2-dihydroquinoline-3-carbonitrile (514 μmol, intermediate 40), 166 mg 4-[4-(trifluoromethoxy)phenoxy]piperidine (617 μmol, CAS 287952-67-4) and 0.27 mL N,N-diisopropylethylamine (1.5 mmol) in 3.0 mL 2-propanol was stirred for 2 h at 90° C. After this time, water was added and the reaction was stirred for some time. The residue was collected by filtration and washed with water and ethanol. The resulting solid was dried to give 231 mg of the title compound (100% yield, 98% purity).

¹H NMR (DMSO-d₆) δ: 7.75 (d, 1H), 7.40 (s, 1H), 7.31 (d, 2H), 7.12-7.19 (m, 3H), 4.77 (dt, 1H), 3.70-3.79 (m, 2H), 3.50-3.59 (m, 5H), 2.47 (s, 3H), 2.16-2.24 (m, 2H), 1.86-1.96 (m, 2H).

LC-MS (Method 1): Rₜ=1.49 min; MS (ESIpos): m/z=458.5 [M+H]⁺

### Example 68

### 7-cyclopropyl-1-methyl-2-oxo-4-[4-(phenylsulfanyl)piperidin-1-yl]-1,2-dihydroquinoline-3-carbonitrile

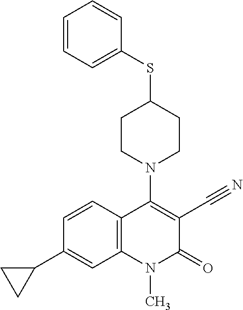 [SMILES: Cn1c(=O)c(C#N)c(N2CCC(Sc3ccccc3)CC2)c2ccc(C3CC3)cc21]

30 mg 7-bromo-1-methyl-2-oxo-4-[4-(phenylsulfanyl)piperidin-1-yl]-1,2-dihydroquinoline-3-carbonitrile (66 μmol, example 55) and 5.8 mg di-μ-iodobis(tri-tert-butylphosphino)dipalladium(I) (6.6 μmol, CAS 166445-62-1) were sealed in a vessel and flushed with argon. 1.1 mL toluene was added and the mixture was stirred at rt. 400 μL bromo(cyclopropyl)zinc (0.50 M, 200 μmol, CAS 126403-68-7) was added dropwise. The mixture was stirred at rt for 1 h. The mixture was filtered via a silica column. The column was washed with dichloromethane and dichloromethane/methanol (9:1). The filtrate was concentrated under reduced pressure. The residue was purified by RP-HPLC (column: X-Bridge C18 5 μm 100×30 mm, mobile phase: acetonitrile/water (0.1 vol. % formic acid)-gradient) to give 24.9 mg of the title compound (100% purity, 91% yield).

¹H NMR (ACETONITRILE-d₃) δ: 7.53-7.68 (m, 1H), 7.33-7.42 (m, 2H), 7.15-7.31 (m, 3H), 6.99-7.11 (m, 1H), 6.78-6.86 (m, 1H), 3.60-3.73 (m, 2H), 3.31-3.45 (m, 3H), 1.91-2.00 (m, 1H), 1.81-1.89 (m, 5H), 1.66-1.80 (m, 2H), 0.96-1.08 (m, 2H), 0.72-0.81 (m, 2H).

LC-MS (Method 1): Rₜ=1.47 min; MS (ESIpos): m/z=416 [M+H]⁺

### Example 69

### 7-butoxy-1-methyl-2-oxo-4-[4-(phenylsulfanyl)piperidin-1-yl]-1,2-dihydroquinoline-3-carbonitrile

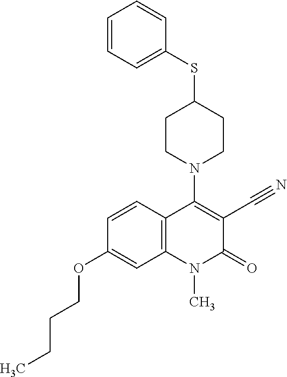 [SMILES: CCCCOc1ccc2c(N3CCC(Sc4ccccc4)CC3)c(C#N)c(=O)n(C)c2c1]

30 mg 7-bromo-1-methyl-2-oxo-4-[4-(phenylsulfanyl)piperidin-1-yl]-1,2-dihydroquinoline-3-carbonitrile (66 μmol, example 55), 5.5 mg (2′-aminobiphenyl-2-yl)(methanesulfonato-kappaO)palladium-di-tert-butyl[3-methoxy-6-methyl-2′,4′,6′-tri(propan-2-yl)biphenyl-2-yl]phosphine (1:1) (6.6 μmol, CAS 1445085-55-1), 3.1 mg di-tert-butyl[3-methoxy-6-methyl-2′,4′,6′-tri(propan-2-yl)biphenyl-2-yl]phosphine (6.6 μmol, CAS 1262046-34-3) and 30 mg cesium carbonate (92 μmol) were sealed in a vessel and flushed with argon. 1.0 mL toluene and 60 μL n-butanol were added and the mixture was stirred overnight at 80° C. The mixture was filtered via a silica column. The column was washed with dichloromethane and dichloromethane/methanol (9:1). The filtrate was concentrated under reduced pressure. The residue was purified by RP-HPLC (column: X-Bridge C18 5 μm 100×30 mm, mobile phase: acetonitrile/water (0.1 vol. % formic acid)-gradient) to give 21.5 mg of the title compound (100% purity, 73% yield).

¹H NMR (ACETONITRILE-d₃) δ: 7.49-7.58 (m, 1H), 7.20-7.37 (m, 2H), 7.02-7.19 (m, 3H), 6.56-6.67 (m, 2H), 3.84-4.00 (m, 2H), 3.49-3.60 (m, 2H), 3.34 (s, 3H), 3.18-3.32 (m, 3H), 1.90 (br d, 2H), 1.51-1.69 (m, 4H), 1.23-1.35 (m, 2H), 0.72-0.81 (m, 3H).

LC-MS (Method 1): Rₜ=1.58 min; MS (ESIpos): m/z=448 [M+H]⁺

### Example 70

### 7-(cyclopropyloxy)-1-methyl-2-oxo-4-(4-phenoxypiperidin-1-yl)-1,2-dihydroquinoline-3-carbonitrile

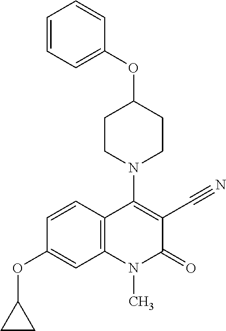 [SMILES: Cn1c(=O)c(C#N)c(N2CCC(Oc3ccccc3)CC2)c2ccc(OC3CC3)cc21]

30 mg 7-bromo-1-methyl-2-oxo-4-(4-phenoxypiperidin-1-yl)-1,2-dihydroquinoline-3-carbonitrile (68 μmol, example 54), 5.7 mg (2′-aminobiphenyl-2-yl)(methanesulfonato-kappaO)palladium-di-tert-butyl[3-methoxy-6-methyl-2′,4′,6′-tri(propan-2-yl)biphenyl-2-yl]phosphine (1:1) (6.8 μmol, CAS 1445085-55-1), 3.2 mg di-tert-butyl[3-methoxy-6-methyl-2′,4′,6′-tri(propan-2-yl)biphenyl-2-yl]phosphine (6.8 μmol, CAS 1262046-34-3) and 31 mg cesium carbonate (96 μmol) were sealed in a vessel and flushed with argon. 1.0 mL toluene and 33 μL cyclopropanol were added and the mixture was stirred overnight at 80° C. The mixture was filtered via a silica column. The column was washed with dichloromethane and dichloromethane/methanol (9:1). The filtrate was concentrated under reduced pressure. The residue was purified by RP-HPLC (column: X-Bridge C18 5 μm 100×30 mm, mobile phase: acetonitrile/water (0.1 vol. % formic acid)-gradient) to give 23.5 mg of the title compound (100% purity, 83% yield).

¹H NMR (ACETONITRILE-d₃) δ: 7.49-7.58 (m, 1H), 7.20-7.37 (m, 2H), 7.02-7.19 (m, 3H), 6.56-6.67 (m, 2H), 3.84-4.00 (m, 2H), 3.49-3.60 (m, 2H), 3.34 (s, 3H), 3.18-3.32 (m, 3H), 1.90 (br d, 2H), 1.51-1.69 (m, 4H), 1.23-1.35 (m, 2H), 0.72-0.81 (m, 3H).

LC-MS (Method 1): Rₜ=1.58 min; MS (ESIpos): m/z=448 [M+H]⁺

### Example 71

### 7-methoxy-1-methyl-2-oxo-4-{4-[4-(trifluoromethoxy)phenoxy]piperidin-1-yl}-1,2-dihydroquinoline-3-carbonitrile

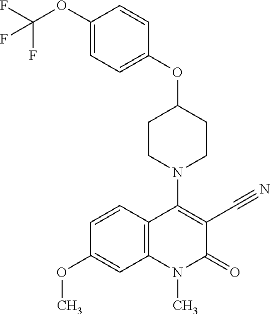 [SMILES: COc1ccc2c(N3CCC(Oc4ccc(OC(F)(F)F)cc4)CC3)c(C#N)c(=O)n(C)c2c1]

30 mg 7-bromo-1-methyl-2-oxo-4-{4-[4-(trifluoromethoxy)phenoxy]piperidin-1-yl}-1,2-dihydroquinoline-3-carbonitrile (57 μmol, example 58), 4.9 mg (2′-amino[biphenyl]-2-yl)(methanesulfonato-kappaO)palladium-di-tert-butyl(2′,4′,6′-triisopropyl-3,6-dimethoxy-[biphenyl]-2-yl)phosphine (1:1) (5.74 μmol, CAS 1536473-72-9), 2.8 mg di-tert-butyl[3,6-dimethoxy-2′,4′,6′-tri(propan-2-yl)biphenyl-2-yl]phosphine (5.7 μmol, CAS 1160861-53-9) and 26 mg cesium carbonate (80 μmol) were sealed in a vessel and flushed with argon. 1.0 mL toluene and 23 μL methanol were added and the mixture was stirred overnight at 60° C. The mixture was filtered via a silica column. The column was washed with dichloromethane/methanol (9:1). The filtrate was concentrated under reduced pressure. The residue was purified by RP-HPLC (column: X-Bridge C18 5 μm 100×30 mm, mobile phase: acetonitrile/water (0.2 vol. % ammonia 32%)-gradient) to give 4.1 mg of the title compound (100% purity, 15% yield).

¹H NMR (ACETONITRILE-d₃) δ: 7.64 (d, 1H), 7.02-7.12 (m, 2H), 6.86-6.94 (m, 2H), 6.66-6.82 (m, 2H), 4.47-4.60 (m, 1H), 3.76 (s, 3H), 3.56-3.67 (m, 2H), 3.42 (s, 3H), 3.33-3.41 (m, 2H), 2.01-2.10 (m, 2H), 1.79-1.85 (m, 2H).

LC-MS (Method 2): Rₜ=1.43 min; MS (ESIpos): m/z=474 [M+H]⁺

### Example 72

### 4-[4-(4-chlorophenoxy)piperidin-1-yl]-1-methyl-7-(oxetan-3-yl)-2-oxo-1,2-dihydroquinoline-3-carbonitrile

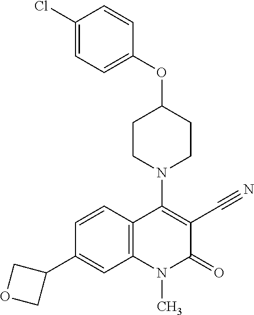 [SMILES: Cn1c(=O)c(C#N)c(N2CCC(Oc3ccc(Cl)cc3)CC2)c2ccc(C3COC3)cc21]

50 mg 7-bromo-4-[4-(4-chlorophenoxy)piperidin-1-yl]-1-methyl-2-oxo-1,2-dihydroquinoline-3-carbonitrile (106 μmol, example 57), 2.4 mg bis{3,5-difluoro-2-[5-(trifluoromethyl)pyridin-2-yl]phenyl}iridium(I)hexafluorophosphate-4,4′-di-tert-butyl-2,2′-bipyridine (1:1:1) (2.1 μmol, CAS 870987-63-6) and 74 μL 2,6-dimethylpyridine (635 μmol) were dissolved in the reaction vial in 1.9 mL 1,2-dimethoxyethane. In a separate vial the Ni-catalyst was prepared by dissolving 1.2 mg 1,2-dimethoxyethane-dichloronickel (1:1) (5.3 μmol) and 1.4 mg 4,4′-di-tert-butyl-2,2′-bipyridine (5.3 μmol) in 1.9 mL 1,2-dimethoxyethane followed by stirring for 5 min. The catalyst solution was syringed to the sealed reaction vial and was degassed with argon for 5 min., then 39.5 μL 3-bromooxetane (476 μmol, CAS 39267-79-3) and 32.6 μL 1,1,1,3,3,3-hexamethyl-2-(trimethylsilyl)trisilane (106 μmol) was added. The vial was placed in a water bath (to keep the temperature below 35° C.) and was subsequently irradiated by two 40 W Kessil LED Aquarium lamps for 16 h. The reaction was quenched with water, extracted with ethyl acetate (3×), dried over sodium sulfate and concentrated in vacuum. The crude material was purificated by flash chromatography (silica, dichloromethane/methanol gradient 0-1%). 20 mg of the title compound were obtained (93% purity, 12% yield).

¹H NMR (DMSO-d₆) δ: 7.84-7.92 (m, 1H), 7.51 (s, 2H), 7.32-7.40 (m, 2H), 7.05-7.12 (m, 2H), 4.93-5.03 (m, 2H), 4.73-4.80 (m, 1H), 4.66-4.72 (m, 2H), 4.36-4.51 (m, 1H), 3.69-3.82 (m, 2H), 3.49-3.62 (m, 5H), 2.15-2.25 (m, 2H), 1.86-1.98 (m, 2H).

LC-MS (Method 2): Rₜ=1.30 min; MS (ESIpos): m/z=450.5 [M+H]⁺

### Example 73

### 4-[4-(3-chlorophenoxy)piperidin-1-yl]-1-methyl-7-(oxetan-3-yl)-2-oxo-1,2-dihydroquinoline-3-carbonitrile

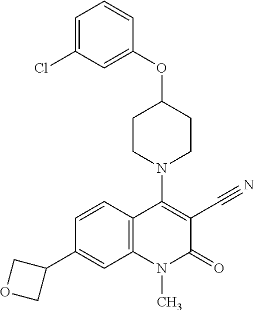 [SMILES: Cn1c(=O)c(C#N)c(N2CCC(Oc3cccc(Cl)c3)CC2)c2ccc(C3COC3)cc21]

50 mg 7-bromo-4-[4-(3-chlorophenoxy)piperidin-1-yl]-1-methyl-2-oxo-1,2-dihydroquinoline-3-carbonitrile (106 μmol, example 60), 2.4 mg bis{3,5-difluoro-2-[5-(trifluoromethyl)pyridin-2-yl]phenyl}iridium(I)hexafluorophosphate-4,4′-di-tert-butyl-2,2′-bipyridine (1:1:1) (2.1 μmol, CAS 870987-63-6) and 74 μL 2,6-dimethylpyridine (635 μmol) were dissolved in the reaction vial in 1.9 mL 1,2-dimethoxyethane. In a separate vial the Ni-catalyst was prepared by dissolving 1.2 mg 1,2-dimethoxyethane-dichloronickel (1:1) (5.3 μmol) and 1.4 mg 4,4′-di-tert-butyl-2,2′-bipyridine (5.3 μmol) in 1.9 mL 1,2-dimethoxyethane followed by stirring for 5 min. The catalyst solution was syringed to the sealed reaction vial and was degassed with argon for 5 min., then 39.5 μL 3-bromooxetane (476 μmol, CAS 39267-79-3) and 32.6 μL 1,1,1,3,3,3-hexamethyl-2-(trimethylsilyl)trisilane (106 μmol) was added. The vial was placed in a water bath (to keep the temperature below 35° C.) and was subsequently irradiated by two 40 W Kessil LED Aquarium lamps for 16 h. The reaction was quenched with water, extracted with ethyl acetate (3×), dried over sodium sulfate and concentrated in vacuum. The crude material was purificated by flash chromatography (silica, dichloromethane/methanol gradient 0-1%). 14 mg of the title compound were obtained (95% purity, 9% yield).

¹H NMR (DMSO-d₆) δ: 7.88 (d, 1H), 7.41-7.51 (m, 2H), 7.25-7.39 (m, 1H), 7.12-7.21 (m, 1H), 6.97-7.06 (m, 2H), 4.95-5.03 (m, 2H), 4.77-4.91 (m, 1H), 4.64-4.74 (m, 2H), 4.36-4.52 (m, 1H), 3.70-3.85 (m, 2H), 3.50-3.62 (m, 5H), 2.14-2.26 (m, 2H), 1.84-2.01 (m, 2H).

LC-MS (Method 2): Rₜ=1.30 min; MS (ESIpos): m/z=450.5 [M+H]⁺

### Example 74

### 4-[4-(3-chlorophenoxy)piperidin-1-yl]-7-(2-methoxyethyl)-1-methyl-2-oxo-1,2-dihydroquinoline-3-carbonitrile

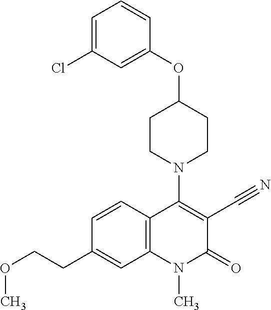 [SMILES: COCCc1ccc2c(N3CCC(Oc4cccc(Cl)c4)CC3)c(C#N)c(=O)n(C)c2c1]

50 mg 7-bromo-4-[4-(3-chlorophenoxy)piperidin-1-yl]-1-methyl-2-oxo-1,2-dihydroquinoline-3-carbonitrile (106 μmol, example 60), 2.4 mg bis{3,5-difluoro-2-[5-(trifluoromethyl)pyridin-2-yl]phenyl}iridium(I)hexafluorophosphate-4,4′-di-tert-butyl-2,2′-bipyridine (1:1:1) (2.1 μmol, CAS 870987-63-6) and 74 μL 2,6-dimethylpyridine (635 μmol) were dissolved in the reaction vial in 1.9 mL 1,2-dimethoxyethane. In a separate vial the Ni-catalyst was prepared by dissolving 1.2 mg 1,2-dimethoxyethane-dichloronickel (1:1) (5.3 μmol) and 1.4 mg 4,4′-di-tert-butyl-2,2′-bipyridine (5.3 μmol) in 1.9 mL 1,2-dimethoxyethane followed by stirring for 5 min. The catalyst solution was syringed to the sealed reaction vial and was degassed with argon for 5 min., then 44.7 μL 1-bromo-2-methoxyethane (476 μmol, CAS 6482-24-2) and 32.6 μL 1,1,1,3,3,3-hexamethyl-2-(trimethylsilyl)trisilane (106 μmol) was added. The vial was placed in a water bath (to keep the temperature below 35° C.) and was subsequently irradiated by two 40 W Kessil LED Aquarium lamps for 16 h. The reaction was quenched with water, extracted with ethyl acetate (3×), dried over sodium sulfate and concentrated in vacuum. The crude material was purificated by flash chromatography (silica, dichloromethane/methanol gradient 0-1%). 20 mg of the title compound were obtained (95% purity, 13% yield).

¹H NMR (DMSO-d₆) δ: 7.74-7.79 (m, 1H), 7.41-7.45 (m, 1H), 7.27-7.36 (m, 1H), 7.19-7.25 (m, 1H), 7.12-7.18 (m, 1H), 6.98-7.05 (m, 2H), 4.73-4.89 (m, 1H), 3.70-3.80 (m, 2H), 3.50-3.64 (m, 7H), 3.22-3.27 (m, 3H), 2.97 (t, 2H), 2.19 (ddd, 2H), 1.84-1.96 (m, 2H).

LC-MS (Method 2): Rₜ=1.40 min; MS (ESIpos): m/z=452.5 [M+H]⁺

### Example 75

### 4-[4-(4-chlorophenoxy)piperidin-1-yl]-7-(2-methoxyethyl)-1-methyl-2-oxo-1,2-dihydroquinoline-3-carbonitrile

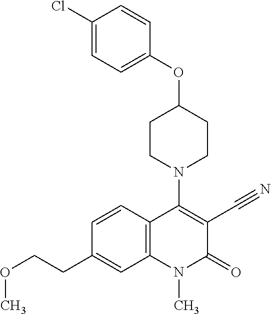 [SMILES: COCCc1ccc2c(N3CCC(Oc4ccc(Cl)cc4)CC3)c(C#N)c(=O)n(C)c2c1]

50 mg 7-bromo-4-[4-(4-chlorophenoxy)piperidin-1-yl]-1-methyl-2-oxo-1,2-dihydroquinoline-3-carbonitrile (106 μmol, example 57), 2.4 mg bis{3,5-difluoro-2-[5-(trifluoromethyl)pyridin-2-yl]phenyl}iridium(I)hexafluorophosphate-4,4′-di-tert-butyl-2,2′-bipyridine (1:1:1) (2.1 μmol, CAS 870987-63-6) and 74 μL 2,6-dimethylpyridine (635 μmol) were dissolved in the reaction vial in 1.9 mL 1,2-dimethoxyethane. In a separate vial the Ni-catalyst was prepared by dissolving 1.2 mg 1,2-dimethoxyethane-dichloronickel (1:1) (5.3 μmol) and 1.4 mg 4,4′-di-tert-butyl-2,2′-bipyridine (5.3 μmol) in 1.9 mL 1,2-dimethoxyethane followed by stirring for 5 min. The catalyst solution was syringed to the sealed reaction vial and the solution was degassed with argon for 5 min., then 44.7 μL 1-bromo-2-methoxyethane (476 μmol, CAS 6482-24-2) and 32.6 μL 1,1,1,3,3,3-hexamethyl-2-(trimethylsilyl)trisilane (106 μmol) was added. The vial was placed in a water bath (to keep the temperature below 35° C.) and was subsequently irradiated by two 40 W Kessil LED Aquarium lamps for 16 h. The reaction was quenched with water, extracted with ethyl acetate (3×), dried over sodium sulfate and concentrated in vacuum. The crude material was purificated by flash chromatography (silica, dichloromethane/methanol gradient 0-1%). 9 mg of the title compound were obtained (95% purity, 6% yield).

¹H NMR (DMSO-d₆) δ: 7.63-7.75 (m, 1H), 7.09-7.41 (m, 4H), 6.91-7.07 (m, 2H), 4.60-4.76 (m, 1H), 3.37-3.75 (m, 9H), 3.16 (s, 3H), 2.88 (br t, 2H), 2.04-2.18 (m, 2H), 1.73-1.88 (m, 2H).

LC-MS (Method 2): Rₜ=1.37 min; MS (ESIpos): m/z=452.4 [M+H]⁺

### Example 76

### 7-(2-methoxyethyl)-1-methyl-2-oxo-4-{4-[3-(trifluoromethoxy)phenoxy]piperidin-1-yl}-1,2-dihydroquinoline-3-carbonitrile

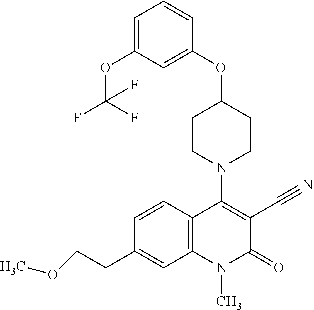 [SMILES: COCCc1ccc2c(N3CCC(Oc4cccc(OC(F)(F)F)c4)CC3)c(C#N)c(=O)n(C)c2c1]

60 mg 7-bromo-1-methyl-2-oxo-4-{4-[3-(trifluoromethoxy)phenoxy]piperidin-1-yl}-1,2-dihydroquinoline-3-carbonitrile (115 μmol, example 61), 2.6 mg bis{3,5-difluoro-2-[5-(trifluoromethyl)pyridin-2-yl]phenyl}iridium(I)hexafluorophosphate-4,4′-di-tert-butyl-2,2′-bi-pyridine (1:1:1) (2.3 μmol, CAS 870987-63-6) and 80 μL 2,6-dimethylpyridine (689 μmol) were dissolved in the reaction vial in 2.1 mL 1,2-dimethoxyethane. In a separate vial the Ni-catalyst was prepared by dissolving 1.3 mg 1,2-dimethoxyethane-dichloronickel (1:1) (5.7 μmol) and 1.5 mg 4,4′-di-tert-butyl-2,2′-bipyridine (5.7 μmol) in 2.1 mL 1,2-dimethoxyethane followed by stirring for 5 min. The catalyst solution was syringed to the sealed reaction vial and the solution was degassed with argon for 5 min., then 49 μL 1-bromo-2-methoxyethane (517 μmol, CAS 6482-24-2) and 35 μL 1,1,1,3,3,3-hexamethyl-2-(trimethylsilyl)trisilane (115 μmol) was added. The vial was placed in a water bath (to keep the temperature below 35° C.) and was subsequently irradiated by two 40 W Kessil LED Aquarium lamps for 16 h. The reaction was quenched with water, extracted with ethyl acetate (3×), dried over sodium sulfate and concentrated in vacuum. The crude material was purificated by flash chromatography (silica, dichloromethane/methanol gradient 0-1%). 21 mg of the title compound were obtained (95% purity, 13% yield).

¹H NMR (DMSO-d₆) δ: 7.73-7.81 (m, 1H), 7.39-7.46 (m, 2H), 7.18-7.28 (m, 1H), 7.02-7.14 (m, 2H), 6.90-7.00 (m, 1H), 4.76-4.93 (m, 1H), 3.70-3.83 (m, 2H), 3.47-3.67 (m, 7H), 3.20-3.27 (m, 3H), 2.92-3.01 (m, 2H), 2.15-2.24 (m, 2H), 1.86-1.99 (m, 2H).

LC-MS (Method 2): Rₜ=1.43 min; MS (ESIpos): m/z=502.5 [M+H]⁺

### Example 77

### 1-methyl-7-(oxetan-3-yl)-2-oxo-4-{4-[3-(trifluoromethoxy)phenoxy]piperidin-1-yl}-1,2-dihydroquinoline-3-carbonitrile

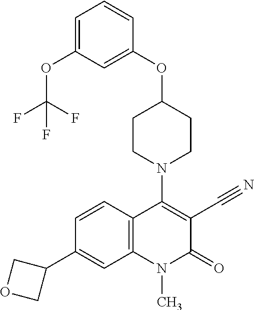 [SMILES: Cn1c(=O)c(C#N)c(N2CCC(Oc3cccc(OC(F)(F)F)c3)CC2)c2ccc(C3COC3)cc21]

60 mg 7-bromo-1-methyl-2-oxo-4-{4-[3-(trifluoromethoxy)phenoxy]piperidin-1-yl}-1,2-dihydroquinoline-3-carbonitrile (115 μmol, example 61), 2.6 mg bis{3,5-difluoro-2-[5-(trifluoromethyl)pyridin-2-yl]phenyl}iridium(I)hexafluorophosphate-4,4′-di-tert-butyl-2,2′-bi-pyridine (1:1:1) (2.3 μmol, CAS 870987-63-6) and 80 μL 2,6-dimethylpyridine (689 μmol) were dissolved in the reaction vial in 2.1 mL 1,2-dimethoxyethane. In a separate vial the Ni-catalyst was prepared by dissolving 1.3 mg 1,2-dimethoxyethane-dichloronickel (1:1) (5.7 μmol) and 1.5 mg 4,4′-di-tert-butyl-2,2′-bipyridine (5.7 μmol) in 2.1 mL 1,2-dimethoxyethane followed by stirring for 5 min. The catalyst solution was syringed to the sealed reaction vial and the solution was degassed with argon for 5 min., then 43 μL 3-bromooxetane (517 μmol, CAS 39267-79-3) and 35 μL 1,1,1,3,3,3-hexamethyl-2-(trimethylsilyl)trisilane (115 μmol) was added. The vial was placed in a water bath (to keep the temperature below 35° C.) and was subsequently irradiated by two 40 W Kessil LED Aquarium lamps for 16 h. The reaction was quenched with water, extracted with ethyl acetate (3×), dried over sodium sulfate and concentrated in vacuum. The crude material was purificated by flash chromatography (silica, dichloromethane/methanol gradient 0-1%). 26 mg of the title compound were obtained (95% purity, 16% yield).

¹H NMR (DMSO-d₆) δ: 7.82-7.92 (m, 1H), 7.39-7.51 (m, 3H), 7.06-7.14 (m, 2H), 6.89-6.99 (m, 1H), 4.94-5.08 (m, 2H), 4.77-4.91 (m, 1H), 4.65-4.75 (m, 2H), 4.36-4.49 (m, 1H), 3.71-3.82 (m, 2H), 3.51-3.64 (m, 5H), 2.16-2.27 (m, 2H), 1.83-1.99 (m, 2H).

LC-MS (Method 2): Rₜ=1.36 min; MS (ESIpos): m/z=500.5 [M+H]⁺

### Example 78

### 7-(dimethylphosphoryl)-1-methyl-2-oxo-4-{4-[3-(trifluoromethoxy)phenoxy]piperidin-1-yl}-1,2-dihydroquinoline-3-carbonitrile

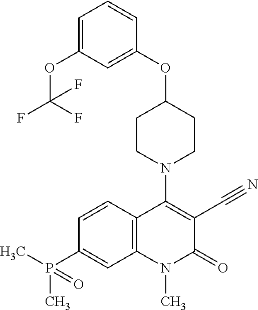 [SMILES: Cn1c(=O)c(C#N)c(N2CCC(Oc3cccc(OC(F)(F)F)c3)CC2)c2ccc(P(C)(C)=O)cc21]

To a suspension of 150 mg 7-bromo-1-methyl-2-oxo-4-{4-[3-(trifluoromethoxy)phenoxy]-piperidin-1-yl}-1,2-dihydroquinoline-3-carbonitrile (287 μmol, example 61), 10 mg (9,9-dimethyl-9H-xanthene-4,5-diyl)bis(diphenylphosphine) (17 μmol), 3.2 mg palladium(II)diacetate (14 μmol) and 67 mg tripotassium phosphate (316 μmol) in 5 mL DMF was added 26.5 mg dimethylphosphine oxide (316 μmol, CAS 7211-39-4) and the mixture was stirred for 6 h at 130° C. Water was added, and the reaction was extracted with ethyl acetate (2×). The organic phase was washed with water and brine and dried over sodium sulfate. After evaporation of the solvent, the residue was purified by RP-HPLC (column: X-Bridge C18 5 μm 100×30 mm, mobile phase: acetonitrile/water (0.2 vol. % ammonia 32%)-gradient) to give 95 mg of the title compound (95% purity, 60% yield).

¹H NMR (DMSO-d₆) δ: 7.93-8.03 (m, 1H), 7.78-7.88 (m, 1H), 7.61-7.73 (m, 1H), 7.40-7.47 (m, 1H), 7.05-7.16 (m, 2H), 6.89-6.99 (m, 1H), 4.79-4.92 (m, 1H), 3.71-3.85 (m, 2H), 3.52-3.67 (m, 5H), 2.15-2.26 (m, 2H), 1.88-1.99 (m, 2H), 1.74 (d, 6H).

LC-MS (Method 2): Rₜ=1.19 min; MS (ESIpos): m/z=520.5 [M+H]⁺

### Example 79

### 7-(dimethylphosphoryl)-1-methyl-2-oxo-4-{4-[4-(trifluoromethoxy)phenoxy]piperidin-1-yl}-1,2-dihydroquinoline-3-carbonitrile

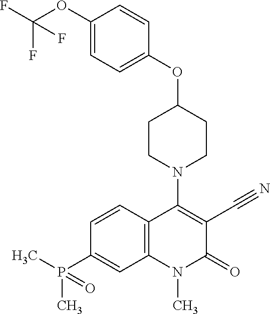 [SMILES: Cn1c(=O)c(C#N)c(N2CCC(Oc3ccc(OC(F)(F)F)cc3)CC2)c2ccc(P(C)(C)=O)cc21]

To a suspension of 140 mg 7-bromo-1-methyl-2-oxo-4-{4-[4-(trifluoromethoxy)phenoxy]-piperidin-1-yl}-1,2-dihydroquinoline-3-carbonitrile (268 μmol, example 58), 9.3 mg (9,9-dimethyl-9H-xanthene-4,5-diyl)bis(diphenylphosphine) (16 μmol), 3 mg palladium(II)diacetate (13 μmol) and 63 mg tripotassium phosphate (295 μmol) in 6 mL DMF was added 24.7 mg dimethylphosphine oxide (295 μmol, CAS 7211-39-4) and the mixture was stirred for 6 h at 130° C. Water was added, and the reaction was extracted with ethyl acetate (2×). The organic phase was washed with water and brine and dried over sodium sulfate. After evaporation of the solvent, the residue was purified by flash chromatography (silica, dichloromethane/methanol gradient 0-5%). The impure product was stirred in ethyl acetate overnight. The solid that precipitated from this procedure was collected by filtration and dried. 49 mg of the title compound were obtained (95% purity, 33% yield).

¹H NMR (DMSO-d₆) δ: 7.95-8.06 (m, 1H), 7.76-7.86 (m, 1H), 7.63-7.70 (m, 1H), 7.25-7.39 (m, 2H), 7.11-7.19 (m, 2H), 4.72-4.86 (m, 1H), 3.73-3.84 (m, 2H), 3.52-3.69 (m, 5H), 2.15-2.26 (m, 2H), 1.87-2.03 (m, 2H), 1.74 (d, 6H).

LC-MS (Method 2): Rₜ=1.16 min; MS (ESIpos): m/z=520.3 [M+H]⁺

### Example 80

### 4-[4-(3-chlorophenoxy)piperidin-1-yl]-1-methyl-7-(4-methylpiperazin-1-yl)-2-oxo-1,2-dihydroquinoline-3-carbonitrile

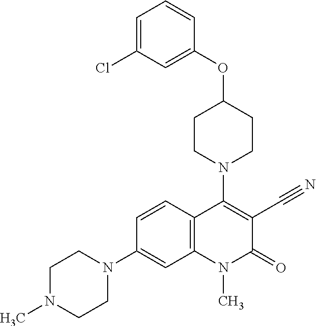 [SMILES: CN1CCN(c2ccc3c(N4CCC(Oc5cccc(Cl)c5)CC4)c(C#N)c(=O)n(C)c3c2)CC1]

A suspension of 90 mg 7-bromo-4-[4-(3-chlorophenoxy)piperidin-1-yl]-1-methyl-2-oxo-1,2-dihydroquinoline-3-carbonitrile (190 μmol, example 60), 23 mg 1-methylpiperazine (228 μmol, CAS 109-01-3), 15 mg chloro(2-dicyclohexylphosphino-2′,4′,6′-triisopropyl-1,1′-biphenyl)[2-(2′-amino-1,1′-biphenyl)]palladium(II) (19.0 μmol) and 124 mg cesium carbonate (381 μmol) in 1,4-dioxane was stirred for 4 h at 110° C. Water was added, and the reaction was extracted with ethyl acetate (2×). The organic phase was washed with water and brine and dried over sodium sulfate. After evaporation of the solvent, the residue was purified by RP-HPLC (column: X-Bridge C18 5 μm 100×30 mm, mobile phase: acetonitrile/water (0.2 vol. % ammonia 32%)-gradient). The impure product was purified by flash chromatography (silica, dichloromethane/methanol gradient 0-1%) to give 5 mg of the title compound (95% purity, 5% yield).

¹H NMR (DMSO-d₆) δ: 7.58-7.68 (m, 1H), 7.27-7.38 (m, 1H), 7.12-7.19 (m, 1H), 6.94-7.05 (m, 3H), 6.62-6.74 (m, 1H), 4.72-4.87 (m, 1H), 3.62-3.79 (m, 2H), 3.46-3.57 (m, 5H), 3.41-3.46 (m, 4H), 2.42-2.46 (m, 4H), 2.21-2.25 (m, 3H), 2.10-2.20 (m, 2H), 1.78-1.94 (m, 2H).

LC-MS (Method 2): Rₜ=1.33 min; MS (ESIpos): m/z=492.4 [M+H]⁺

### Example 81

### 7-(2-methoxyethoxy)-1-methyl-2-oxo-4-{4-[4-(trifluoromethoxy)phenoxy]piperidin-1-yl}-1,2-dihydroquinoline-3-carbonitrile

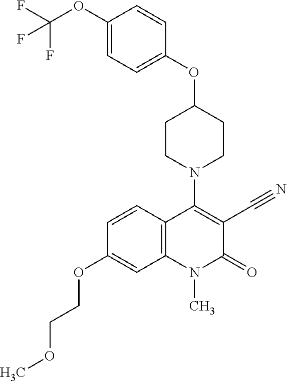 [SMILES: COCCOc1ccc2c(N3CCC(Oc4ccc(OC(F)(F)F)cc4)CC3)c(C#N)c(=O)n(C)c2c1]

A mixture of 100 mg 7-hydroxy-1-methyl-2-oxo-4-{4-[4-(trifluoromethoxy)phenoxy]piperidin-1-yl}-1,2-dihydroquinoline-3-carbonitrile (218 μmol, example 62), 200 μL 1-bromo-2-methoxyethane (2.2 mmol, CAS 6482-24-2) and 60 mg potassium carbonate (435 μmol) in 5.0 mL acetonitrile was refluxed for 2 h. Water was added. The mixture was extracted with dichloromethane. The combined organic phases were washed with water and brine, dried and concentrated under reduced pressure. The residue was purified by flash chromatography (silica, dichloromethane/methanol gradient 0-3%) to give 75 mg of the title compound (98% purity, 65% yield).

¹H NMR (DMSO-d₆) δ: 7.74-7.81 (m, 1H), 7.27-7.36 (m, 2H), 7.10-7.19 (m, 2H), 6.91-7.03 (m, 2H), 4.69-4.87 (m, 1H), 4.29 (dd, 2H), 3.65-3.82 (m, 4H), 3.49-3.57 (m, 5H), 3.32 (s, 3H), 2.11-2.26 (m, 2H), 1.83-1.96 (m, 2H).

LC-MS (Method 2): Rₜ=1.39 min; MS (ESIpos): m/z=518.5 [M+H]⁺

### Example 82

### 1-methyl-2-oxo-7-(2-oxopyrrolidin-1-yl)-4-{4-[4-(trifluoromethoxy)phenoxy]piperidin-1-yl}-1,2-dihydroquinoline-3-carbonitrile

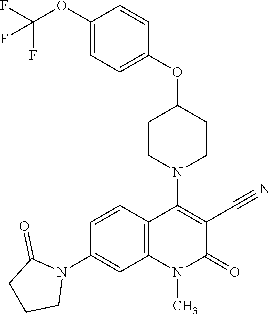 [SMILES: Cn1c(=O)c(C#N)c(N2CCC(Oc3ccc(OC(F)(F)F)cc3)CC2)c2ccc(N3CCCC3=O)cc21]

A suspension of 194 mg 7-bromo-1-methyl-2-oxo-4-{4-[4-(trifluoromethoxy)phenoxy]piperidin-1-yl}-1,2-dihydroquinoline-3-carbonitrile (371 μmol, example 58), 63 mg pyrrolidin-2-one (743 μmol, CAS 616-45-5), 160 μL N,N′-dimethylethane-1,2-diamine (1.5 mmol), 14 mg copper(I)iodide (74 μmol) and 113 mg potassium carbonate (817 μmol) in 6.3 mL toluene was stirred overnight at 110° C. The mixture was cooled down to rt, water was added and the mixture was extracted with dichloromethane. The combined organic phases were washed with brine, filtered (using a waterresistant filter) and concentrated under reduced pressure. The residue was purified by flash chromatography (silica, dichloromethane/ethanol gradient 0-5%). The impure product was stirred in ethanol, the precipitate was collected by filtration and dried in vacuum. 156 mg of the title compound were obtained (76% yield, 95% purity).

¹H NMR (DMSO-d₆) δ: 7.76-7.90 (m, 2H), 7.63-7.72 (m, 1H), 7.25-7.38 (m, 2H), 7.10-7.20 (m, 2H), 4.68-4.89 (m, 1H), 3.88-4.06 (m, 2H), 3.68-3.82 (m, 2H), 3.46-3.65 (m, 5H), 2.55-2.63 (m, 2H), 2.15-2.28 (m, 2H), 2.06-2.15 (m, 2H), 1.85-2.00 (m, 2H).

LC-MS (Method 4): Rₜ=1.31 min; MS (ESIpos): m/z=527 [M+H]⁺

### Example 83

### 1-methyl-2-oxo-7-(2-oxopyrrolidin-1-yl)-4-[4-(phenylsulfanyl)piperidin-1-yl]-1,2-dihydroquinoline-3-carbonitrile

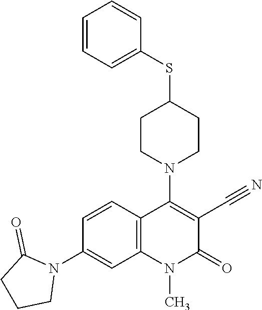 [SMILES: Cn1c(=O)c(C#N)c(N2CCC(Sc3ccccc3)CC2)c2ccc(N3CCCC3=O)cc21]

A suspension of 219 mg 7-bromo-1-methyl-2-oxo-4-[4-(phenylsulfanyl)piperidin-1-yl]-1,2-dihydroquinoline-3-carbonitrile (482 μmol, example 55), 82 mg pyrrolidin-2-one (964 μmol, CAS 616-45-5), 210 μL N,N′-dimethylethane-1,2-diamine (1.9 mmol), 18 mg copper(I)iodide (96 μmol) and 147 mg potassium carbonate (1.1 mmol) in 8.2 mL toluene was stirred overnight at 110′C. The mixture was cooled down to rt, water was added and the mixture was extracted with dichloromethane. The combined organic phases were washed with brine, filtered (using a waterresistant filter) and concentrated under reduced pressure. The residue was purified by flash chromatography (silica, dichloromethane/ethanol gradient 0-5%). The impure product was stirred in ethanol, the precipitate was collected by filtration and dried in vacuum. 180 mg of the title compound were obtained (77% yield, 95% purity).

¹H NMR (DMSO-d₆) δ: 7.75-7.84 (m, 2H), 7.61-7.72 (m, 1H), 7.44-7.53 (m, 2H), 7.33-7.41 (m, 2H), 7.22-7.31 (m, 1H), 3.89-4.03 (m, 2H), 3.70-3.82 (m, 2H), 3.60-3.70 (m, 1H), 3.42-3.58 (m, 5H), 2.55-2.64 (m, 2H), 2.03-2.17 (m, 4H), 1.73-1.87 (m, 2H).

LC-MS (Method 4): Rₜ=1.22 min; MS (ESIpos): m/z=459 [M+H]⁺

### Example 84

### 4-[4-(4-fluorophenoxy)piperidin-1-yl]-1-methyl-2-oxo-7-(2-oxopyrrolidin-1-yl)-1,2-dihydroquinoline-3-carbonitrile

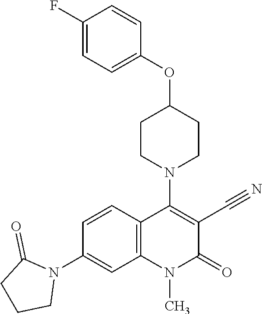 [SMILES: Cn1c(=O)c(C#N)c(N2CCC(Oc3ccc(F)cc3)CC2)c2ccc(N3CCCC3=O)cc21]

A suspension of 75 mg 7-bromo-4-[4-(4-fluorophenoxy)piperidin-1-yl]-1-methyl-2-oxo-1,2-dihydroquinoline-3-carbonitrile (164 μmol, example 56), 28 mg pyrrolidin-2-one (329 μmol, CAS 616-45-5), 71 μL N,N′-dimethylethane-1,2-diamine (660 μmol), 6.3 mg copper(I)iodide (33 μmol) and 50 mg potassium carbonate (362 μmol) in 2.8 mL toluene was stirred overnight at 110′C. The mixture was cooled down to rt, water was added and the mixture was extracted with dichloromethane. The combined organic phases were washed with brine, filtered (using a waterresistant filter) and concentrated under reduced pressure. The residue was purified by flash chromatography (silica, dichloromethane/ethanol gradient 0-5%). The impure product was stirred in ethanol, the precipitate was collected by filtration and dried in vacuum. 63 mg of the title compound were obtained (79% yield, 95% purity).

¹H NMR (DMSO-d₆) δ: 7.78-7.94 (m, 2H), 7.62-7.74 (m, 1H), 7.01-7.24 (m, 4H), 4.64-4.75 (m, 1H), 3.97 (t, 2H), 3.69-3.82 (m, 2H), 3.48-3.63 (m, 5H), 2.58-2.62 (m, 2H), 2.05-2.23 (m, 4H), 1.82-1.99 (m, 2H).

LC-MS (Method 4): Rₜ=1.16 min; MS (ESIpos): m/z=461 [M+H]⁺

### Example 85

### 4-[4-(4-chlorophenoxy)piperidin-1-yl]-1-methyl-2-oxo-7-(2-oxopyrrolidin-1-yl)-1,2-dihydroquinoline-3-carbonitrile

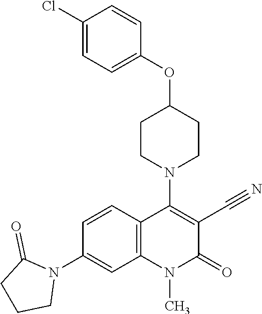 [SMILES: Cn1c(=O)c(C#N)c(N2CCC(Oc3ccc(Cl)cc3)CC2)c2ccc(N3CCCC3=O)cc21]

A suspension of 154 mg 7-bromo-4-[4-(4-chlorophenoxy)piperidin-1-yl]-1-methyl-2-oxo-1,2-dihydroquinoline-3-carbonitrile (164 μmol, example 57), 55 mg pyrrolidin-2-one (651 μmol, CAS 616-45-5), 140 μL N,N′-dimethylethane-1,2-diamine (1.3 mmol), 12 mg copper(I)iodide (65 μmol) and 99 mg potassium carbonate (717 μmol) in 5.6 mL toluene was stirred overnight at 110′C. The mixture was cooled down to rt, water was added and the mixture was extracted with dichloromethane. The combined organic phases were washed with brine, filtered (using a waterresistant filter) and concentrated under reduced pressure. The residue was purified by flash chromatography (silica, dichloromethane/ethanol gradient 0-5%). The impure product was stirred in ethanol, the precipitate was collected by filtration and dried in vacuum. 125 mg of the title compound were obtained (76% yield, 95% purity).

¹H NMR (DMSO-d₆) δ: 7.77-7.88 (m, 2H), 7.63-7.70 (m, 1H), 7.31-7.40 (m, 2H), 7.02-7.14 (m, 2H), 4.69-4.86 (m, 1H), 3.91-4.01 (m, 2H), 3.69-3.80 (m, 2H), 3.47-3.62 (m, 5H), 2.55-2.63 (m, 2H), 2.04-2.26 (m, 4H), 1.83-1.97 (m, 2H).

LC-MS (Method 4): Rₜ=1.25 min; MS (ESIpos): m/z=477 [M+H]⁺

### Example 86

### 1-methyl-2-oxo-7-(2-oxopyrrolidin-1-yl)-4-{4-[4-(trifluoromethyl)phenoxy]piperidin-1-yl}-1,2-dihydroquinoline-3-carbonitrile

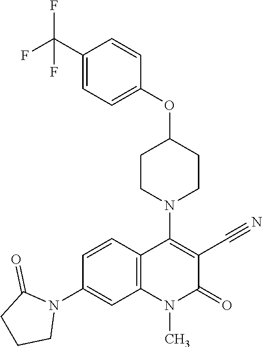 [SMILES: Cn1c(=O)c(C#N)c(N2CCC(Oc3ccc(C(F)(F)F)cc3)CC2)c2ccc(N3CCCC3=O)cc21]

A suspension of 155 mg 7-bromo-1-methyl-2-oxo-4-{4-[4-(trifluoromethyl)phenoxy]piperidin-1-yl}-1,2-dihydroquinoline-3-carbonitrile (306 μmol, example 59), 52 mg pyrrolidin-2-one (612 μmol, CAS 616-45-5), 130 μL N,N′-dimethylethane-1,2-diamine (1.2 mmol), 12 mg copper(I)iodide (61.2 μmol) and 93 mg potassium carbonate (673 μmol) in 5.2 mL toluene was stirred overnight at 110° C. The mixture was cooled down to rt, water was added and the mixture was extracted with dichloromethane. The combined organic phases were washed with brine, filtered (using a waterresistant filter) and concentrated under reduced pressure. The residue was purified by flash chromatography (silica, dichloromethane/ethanol gradient 0-5%). The impure product was stirred in ethanol, the precipitate was collected by filtration and dried in vacuum. 105 mg of the title compound were obtained (64% yield, 95% purity).

¹H NMR (DMSO-d₆) δ: 7.75-7.89 (m, 2H), 7.63-7.73 (m, 3H), 7.20-7.31 (m, 2H), 4.81-4.99 (m, 1H), 3.91-4.03 (m, 2H), 3.69-3.83 (m, 2H), 3.47-3.63 (m, 5H), 2.55-2.63 (m, 2H), 2.19-2.28 (m, 2H), 2.05-2.16 (m, 2H), 1.88-2.00 (m, 2H).

LC-MS (Method 4): Rₜ=1.29 min; MS (ESIpos): m/z=511 [M+H]⁺

### Example 87

### 7-cyclopropyl-1-methyl-2-oxo-4-{4-[4-(trifluoromethyl)phenoxy]piperidin-1-yl}-1,2-dihydroquinoline-3-carbonitrile

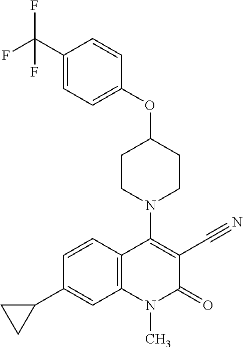 [SMILES: Cn1c(=O)c(C#N)c(N2CCC(Oc3ccc(C(F)(F)F)cc3)CC2)c2ccc(C3CC3)cc21]

mg 7-bromo-1-methyl-2-oxo-4-{4-[4-(trifluoromethyl)phenoxy]piperidin-1-yl}-1,2-dihydroquinoline-3-carbonitrile (59 μmol, example 59) and 5.2 mg di-μ-iodobis(tri-tert-butylphosphino)dipalladium(I) (5.9 μmol, CAS 166445-62-1) were sealed in a vessel and flushed with argon. 1.0 mL toluene was added and the mixture was stirred at rt. 360 μL bromo(cyclopropyl)zinc (0.50 M, 210 μmol, CAS 126403-68-7) was added dropwise. The mixture was stirred at rt for 1 h. The mixture was filtered via a silica column. The column was washed with dichloromethane and dichloromethane/methanol (9:1). The filtrate was concentrated under reduced pressure. The residue was purified by RP-HPLC (instrument: Waters Autopurificationsystem; column: YMC Triart C18 5μ 150×50 mm; eluent A: water (0.1 vol. % formic acid 99%), eluent B: acetonitrile; gradient: 0.00-0.50 min. 60% B, (50->100 mL/min), 0.51-13.0 min. 60-84% B (100 mL/min), DAD scan: 210-400 nm) to give 3.0 mg of the title compound (98% purity, 11% yield).

¹H NMR (ACETONITRILE-d₃) δ: 7.71-7.79 (m, 1H), 7.59-7.67 (m, 2H), 7.09-7.19 (m, 3H), 6.90-6.99 (m, 1H), 4.80 (tt, 1H), 3.72-3.88 (m, 2H), 3.52-3.64 (m, 5H), 2.25 (ddt, 2H), 2.05-2.10 (m, 1H), 1.97-2.04 (m, 2H), 1.08-1.15 (m, 2H), 0.83-0.89 (m, 2H).

LC-MS (Method 2): Rₜ=1.52 min; MS (ESIpos): m/z=468 [M+H]⁺

### Example 88

### 1,7-dimethyl-2-oxo-4-(4-phenoxypiperidin-1-yl)-1,2-dihydroquinoline-3-carbonitrile

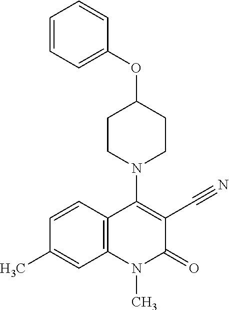 [SMILES: Cc1ccc2c(N3CCC(Oc4ccccc4)CC3)c(C#N)c(=O)n(C)c2c1]

30 mg 7-bromo-1-methyl-2-oxo-4-(4-phenoxypiperidin-1-yl)-1,2-dihydroquinoline-3-carbonitrile (68 μmol, example 54), 21 mg methylboronic acid (342 μmol, CAS 13061-96-6), 28 mg potassium carbonate (205 μmol) and 5.6 mg [1,1′-Bis(diphenylphosphino)ferrocene]dichloropalladium(II)-complex with dichloromethane (6.84 μmol, CAS 95464-05-4) were sealed in a vessel and degassed with argon. 600 μL 1,4-dioxane and 300 μL water (both degassed with argon) were added and the mixture was stirred at 130° C. for 1 h. The mixture was filtered via a water repellent filter. The filter was washed with dichloromethane. The filtrate was concentrated under reduced pressure. The residue was purified by RP-HPLC (column: X-Bridge C18 5 μm 100×30 mm, mobile phase: acetonitrile/water (0.2 vol. % ammonia 32%)-gradient) to give 16.5 mg of the title compound (100% purity, 65% yield).

¹H NMR (ACETONITRILE-d₃) δ: 7.61 (d, 1H), 7.09-7.19 (m, 3H), 6.92-7.01 (m, 1H), 6.76-6.88 (m, 3H), 4.44-4.60 (m, 1H), 3.59-3.70 (m, 2H), 3.33-3.47 (m, 5H), 2.32 (s, 3H), 2.01-2.12 (m, 2H), 1.79-1.85 (m, 2H).

LC-MS (Method 2): Rₜ=1.34 min; MS (ESIpos): m/z=374.3 [M+H]⁺

### Example 89

### 7-methoxy-1-methyl-2-oxo-4-(4-phenoxypiperidin-1-yl)-1,2-dihydroquinoline-3-carbonitrile

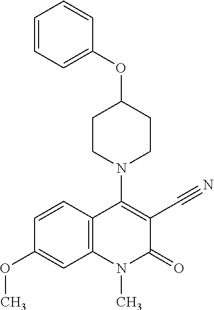 [SMILES: COc1ccc2c(N3CCC(Oc4ccccc4)CC3)c(C#N)c(=O)n(C)c2c1]

30 mg 7-bromo-1-methyl-2-oxo-4-(4-phenoxypiperidin-1-yl)-1,2-dihydroquinoline-3-carbonitrile (68 μmol, example 54), 5.9 mg (2′-amino[biphenyl]-2-yl)(methanesulfonato-kappaO)palladium-di-tert-butyl(2′,4′,6′-triisopropyl-3,6-dimethoxy[biphenyl]-2-yl)phosphine (1:1) (6.84 μmol, CAS 1536473-72-9), 31 mg potassium carbonate (96 μmol) and 3.3 mg di-tert-butyl[3,6-dimethoxy-2′,4′,6′-tri(propan-2-yl)biphenyl-2-yl]phosphine (6.8 μmol, CAS 1160861-53-9) were sealed in a vessel and degassed with argon. 1.0 mL toluene and 28 μL methanol were added and the mixture was stirred overnight at 60° C. The mixture was filtered via a silica column. The column was washed with dichloromethane/methanol (9:1). The filtrate was concentrated under reduced pressure. The residue was purified by RP-HPLC (column: X-Bridge C18 5 μm 100×30 mm, mobile phase: acetonitrile/water (0.2 vol. % ammonia 32%)-gradient) to give 19.2 mg of the title compound (100% purity, 72% yield).

¹H NMR (ACETONITRILE-d₃) δ: 7.49-7.60 (m, 1H), 6.99-7.12 (m, 2H), 6.56-6.82 (m, 5H), 4.43 (tt, 1H), 3.67 (s, 3H), 3.44-3.59 (m, 2H), 3.24-3.36 (m, 5H), 1.91-2.00 (m, 2H), 1.69-1.75 (m, 2H).

LC-MS (Method 2): Rₜ=1.31 min; MS (ESIpos): m/z=390.3 [M+H]⁺

### Example 90

### 7-methoxy-1-methyl-2-oxo-4-[4-(phenylsulfanyl)piperidin-1-yl]-1,2-dihydroquinoline-3-carbonitrile

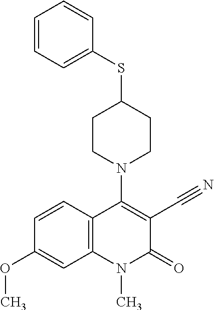 [SMILES: COc1ccc2c(N3CCC(Sc4ccccc4)CC3)c(C#N)c(=O)n(C)c2c1]

mg 7-bromo-1-methyl-2-oxo-4-[4-(phenylsulfanyl)piperidin-1-yl]-1,2-dihydroquinoline-3-carbonitrile (66 μmol, example 55), 5.6 mg (2′-amino[biphenyl]-2-yl)(methanesulfonato-kappaO)palladium-di-tert-butyl(2′,4′,6′-triisopropyl-3,6-dimethoxy[biphenyl]-2-yl)phosphine (1:1) (6.6 μmol, CAS 1536473-72-9), 30 mg potassium carbonate (92 μmol) and 3.3 mg di-tert-butyl[3,6-dimethoxy-2′,4′,6′-tri(propan-2-yl)biphenyl-2-yl]phosphine (6.6 μmol, CAS 1160861-53-9) were sealed in a vessel and degassed with argon. 1.0 mL toluene and 27 μL methanol were added and the mixture was stirred overnight at 60° C. The mixture was filtered via a silica column. The column was washed with dichloromethane/methanol (9:1). The filtrate was concentrated under reduced pressure. The residue was purified by RP-HPLC (column: X-Bridge C18 5 μm 100×30 mm, mobile phase: acetonitrile/water (0.2 vol. % ammonia 32%)-gradient) to give 8.3 mg of the title compound (98% purity, 30% yield).

¹H NMR (ACETONITRILE-d₃) δ: 7.72-7.79 (m, 1H), 7.41-7.53 (m, 2H), 7.24-7.39 (m, 3H), 6.81-6.90 (m, 2H), 3.92 (s, 3H), 3.71-3.80 (m, 2H), 3.57 (s, 3H), 3.41-3.55 (m, 3H), 2.09-2.14 (m, 2H), 1.78-1.89 (m, 2H).

LC-MS (Method 2): Rₜ=1.36 min; MS (ESIpos): m/z=406.3 [M+H]⁺

### Example 91

### 4-(4-benzylpiperidin-1-yl)-1-methyl-2-oxo-1,2-dihydroquinoline-3-carbonitrile

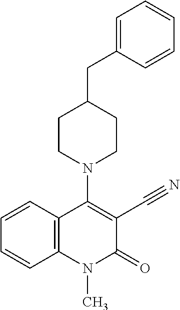 [SMILES: Cn1c(=O)c(C#N)c(N2CCC(Cc3ccccc3)CC2)c2ccccc21]

To a solution of 100 mg 4-chloro-1-methyl-2-oxo-1,2-dihydroquinoline-3-carbonitrile (412 μmol, CAS 150617-68-8, synthesis described in WO2012009649, example 1—compound Ill) and 79 mg 4-benzylpiperidine (453 μmol, CAS 31252-42-3) in 2.5 mL DMSO was added 92 μL triethylamine (660 μmol) and the mixture was stirred for 2 h at 90° C. and overnight at rt. The reaction was filtered and was purified by RP-HPLC (column: X-Bridge C18 5 μm 100×30 mm, mobile phase: acetonitrile/water (0.2 vol. % ammonia 32%)-gradient) to give 58 mg of the title compound (95% purity, 37% yield).

¹H NMR (DMSO-d₆) δ: 7.78-7.86 (m, 1H), 7.66-7.77 (m, 1H), 7.52-7.59 (m, 1H), 7.15-7.39 (m, 6H), 3.73 (br d, 2H), 3.56 (s, 3H), 3.32-3.33 (m, 2H), 2.60-2.65 (m, 2H), 1.80-2.01 (m, 1H), 1.67-1.79 (m, 2H), 1.44-1.56 (m, 2H).

LC-MS (Method 2): Rₜ=1.36 min; MS (ESIpos): m/z=358.7 [M+H]⁺

### Example 92

### 4-{4-[(4-fluorophenyl)methyl]piperidin-1-yl}-1-methyl-2-oxo-1,2-dihydroquinoline-3-carbonitrile

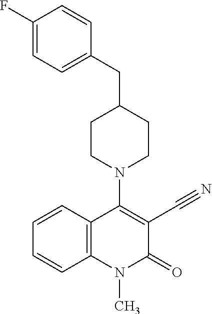 [SMILES: Cn1c(=O)c(C#N)c(N2CCC(Cc3ccc(F)cc3)CC2)c2ccccc21]

A suspension of 100 mg 4-chloro-1-methyl-2-oxo-1,2-dihydroquinoline-3-carbonitrile (457 μmol, CAS 150617-68-8, synthesis described in WO2012009649, example 1—compound Ill), 106 mg 4-[(4-fluorophenyl)methyl]piperidine (549 μmol, CAS 92822-02-1) and 0.24 mL N,N-diisopropylethylamine (1.4 mmol) in 2 mL 2-propanol was stirred for 2 h at 90° C. After this time, water was added and the reaction was stirred for some time. The residue was collected by filtration and washed with ethanol. The resulting solid was dried to give 155 mg of the title compound (95% yield, 86% purity).

¹H NMR (DMSO-d₆) δ: 7.67-7.86 (m, 2H), 7.51-7.62 (m, 1H), 7.23-7.38 (m, 3H), 7.06-7.19 (m, 2H), 3.67-3.79 (m, 2H), 3.55 (s, 3H), 3.29 (br s, 2H), 2.62 (d, 2H), 1.78-1.94 (m, 1H), 1.73 (br d, 2H), 1.41-1.57 (m, 2H).

LC-MS (Method 1): Rₜ=1.37 min; MS (ESIpos): m/z=376 [M+H]⁺

### Example 93

### 4-{4-[(1,3-benzothiazol-2-yl)methyl]piperidin-1-yl}-1-methyl-2-oxo-1,2-dihydroquinoline-3-carbonitrile

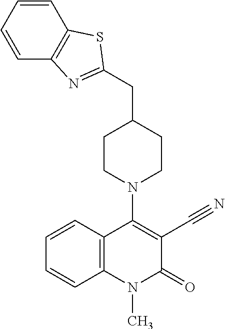 [SMILES: Cn1c(=O)c(C#N)c(N2CCC(Cc3nc4ccccc4s3)CC2)c2ccccc21]

A solution of 100 mg [1-(3-cyano-1-methyl-2-oxo-1,2-dihydroquinolin-4-yl)piperidin-4-yl]acetic acid (307 μmol, intermediate 42), 33 μL 2-aminobenzenethiol (310 μmol, CAS 137-07-5), 220 μL T3P (50% purity in ethyl acetate, 370 μmol) and 110 μL N,N-diisopropylethylamine (0.61 mmol) in 1.0 mL N,N-dimethylacetamide was stirred for 2 h at 100° C. The mixture was cooled down to rt and was concentrated under reduced pressure. The residue was purified by RP-HPLC (column: Chromatorex 125×30 mm, 10 μm mobile phase: acetonitrile/water (0.2 vol. % ammonia 32%)-gradient) to give 52.6 mg of the title compound (95% purity, 39% yield).

¹H NMR (DMSO-d₆) δ: 8.04-8.13 (m, 1H), 7.92-8.00 (m, 1H), 7.80-7.88 (m, 1H), 7.67-7.78 (m, 1H), 7.45-7.60 (m, 2H), 7.38-7.45 (m, 1H), 7.29-7.38 (m, 1H), 3.70-3.86 (m, 2H), 3.58 (s, 3H), 3.37-3.46 (m, 2H), 3.13-3.27 (m, 2H), 2.14-2.29 (m, 1H), 1.91 (br d, 2H), 1.54-1.70 (m, 2H).

LC-MS (Method 1): Rₜ=1.29 min; MS (ESIpos): m/z=416 [M+H]⁺

### Example 94

### 4-{4-[(2-methoxyphenyl)methyl]piperidin-1-yl}-1-methyl-2-oxo-1,2-dihydroquinoline-3-carbonitrile

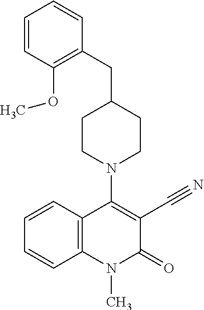 [SMILES: COc1ccccc1CC1CCN(c2c(C#N)c(=O)n(C)c3ccccc23)CC1]

A suspension of 36 mg 4-chloro-1-methyl-2-oxo-1,2-dihydroquinoline-3-carbonitrile (162 μmol, CAS 150617-68-8, synthesis described in WO2012009649, example 1—compound III), 100 mg 4-[(2-methoxyphenyl)methyl]piperidine (487 μmol, CAS 37581-33-2) and 23 μL triethylamine (160 μmol) in 1 mL 2-propanol was stirred for 6 h at 90° C. The mixture was cooled down to rt, diisopropyl ether was added and for 5 min. sonicated. The solid that precipitated from this procedure was collected by filtration and dried. 85 mg of the title compound were obtained (95% purity, 128% yield).

¹H NMR (DMSO-d₆) δ: 7.77-7.88 (m, 1H), 7.67-7.76 (m, 1H), 7.51-7.61 (m, 1H), 7.27-7.40 (m, 1H), 7.14-7.26 (m, 2H), 6.95-7.05 (m, 1H), 6.84-6.92 (m, 1H), 3.80 (s, 3H), 3.67-3.76 (m, 2H), 3.56 (s, 3H), 2.57-2.65 (m, 2H), 1.81-1.97 (m, 1H), 1.66-1.77 (m, 2H), 1.42-1.57 (m, 2H).

LC-MS (Method 2): Rₜ=1.34 min; MS (ESIpos): m/z=388.7 [M+H]⁺

### Example 95

### 4-{4-[(4-cyanophenyl)methyl]piperidin-1-yl}-1-methyl-2-oxo-1,2-dihydroquinoline-3-carbonitrile

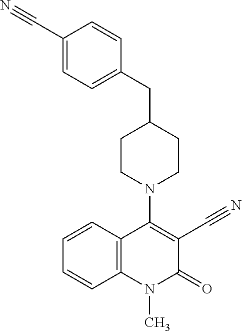 [SMILES: Cn1c(=O)c(C#N)c(N2CCC(Cc3ccc(C#N)cc3)CC2)c2ccccc21]

A suspension of 100 mg 4-chloro-1-methyl-2-oxo-1,2-dihydroquinoline-3-carbonitrile (457 μmol, CAS 150617-68-8, synthesis described in WO2012009649, example 1—compound Ill), 135 mg 4-[(piperidin-4-yl)methyl]benzonitrile hydrogen chloride salt (1:1) (549 μmol, CAS 333987-04-5) and 0.24 mL N,N-diisopropylethylamine (1.4 mmol) in 2.0 mL 2-propanol was stirred for 2 h at 90° C. After this time, water was added and the reaction was stirred for some time. The residue was collected by filtration and washed with ethanol. The resulting solid was dried. The impure product was purified by preparative TLC (dichloromethane/ethanol; 95:5) to give 117 mg of the title compound (64% yield, 95% purity).

¹H NMR (DMSO-d₆) δ: 7.69-7.90 (m, 4H), 7.53-7.61 (m, 1H), 7.41-7.52 (m, 2H), 7.26-7.38 (m, 1H), 3.67-3.84 (m, 2H), 3.56 (s, 3H), 3.29 (br s, 2H), 2.69-2.81 (m, 2H), 1.85-2.05 (m, 1H), 1.65-1.78 (m, 2H), 1.43-1.61 (m, 2H).

LC-MS (Method 1): Rₜ=1.26 min; MS (ESIpos): m/z=383 [M+H]⁺

### Example 96

### 1-methyl-2-oxo-7-(2-oxopyrrolidin-1-yl)-4-{4-[4-(trifluoromethoxy)phenoxy]piperidin-1-yl}-1,2-dihydroquinoline-3-carboxamide

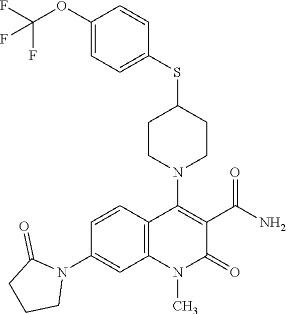 [SMILES: Cn1c(=O)c(C(N)=O)c(N2CCC(Sc3ccc(OC(F)(F)F)cc3)CC2)c2ccc(N3CCCC3=O)cc21]

A mixture of 50 mg 1-methyl-2-oxo-7-(2-oxopyrrolidin-1-yl)-4-{4-[4-(trifluoromethoxy)-phenoxy]piperidin-1-yl}-1,2-dihydroquinoline-3-carbonitrile (90 μmol, example 82), 5.1 mg palladium(II)diacetate (23 μmol) and 53 mg N-[(1E)-ethylidene]hydroxylamine (0.9 mmol, CAS 107-29-9) in 5 mL ethanol was stirred for 4 h at 80° C. Water was added and the reaction was extracted with ethyl acetate (2×). The organic phase was washed with brine and dried over sodium sulfate. After evaporation, the residue was purified by RP-HPLC (column: X-Bridge C18 5 μm 100×30 mm, mobile phase: acetonitrile/water (0.2 vol. % ammonia 32%)-gradient) to give 34 mg of the title compound (95% purity, 66% yield).

¹H NMR (DMSO-d₆) δ: 7.80-7.94 (m, 2H), 7.57-7.75 (m, 2H), 7.40-7.49 (m, 1H), 7.24-7.33 (m, 2H), 7.05-7.15 (m, 2H), 4.54-4.72 (m, 1H), 3.89-4.04 (m, 2H), 3.56 (s, 3H), 3.34-3.41 (m, 2H), 3.09-3.20 (m, 2H), 2.53-2.61 (m, 2H), 2.05-2.19 (m, 4H), 1.78-1.92 (m, 2H).

LC-MS (Method 2): Rₜ=1.20 min; MS (ESIpos): m/z=545.5 [M+H]⁺

TABLE 4
synthetic precedure for compound in analogy to example 96.

| | | | Starting | |
|---|---|---|---|---|
| Example | Structure | IUPAC-Name | Materials | Analytics |
| 97 | 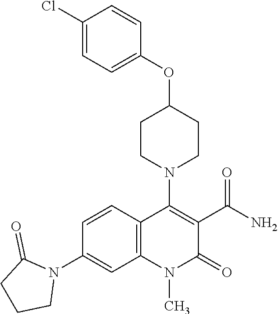 [SMILES: Cn1c(=O)c(C(N)=O)c(N2CCC(Oc3ccc(Cl)cc3)CC2)c2ccc(N3CCCC3=O)cc21] | 4-[4-(4- chlorophenoxy) piperidin-1-yl]- 1-methyl-2- oxo-7-(2- oxopyrrolidin-1- yl)-1,2- dihydroquinoline-3- carboxamide | 4-[4-(4- chlorophenoxy) piperidin-1- yl]-1-methyl- 2-oxo-7-(2- oxopyrrolidin- 1-yl)-1,2- dihydroquinoline- 3-carbonitrile (example 85) | ¹H NMR (DMSO-d₆) δ: 7.77-7.95 (m, 2H), 7.55- 7.72 (m, 2H), 7.38-7.51 (m, 1H), 7.28-7.38 (m, 2H), 6.99-7.10 (m, 2H), 4.53-4.68 (m, 1H), 3.89- 4.03 (m, 2H), 3.58 (s, 3H), 3.08-3.22 (m, 2H), 2.53- 2.60 (m, 4H), 2.05-2.17 (m, 4H), 1.76-1.91 (m, 2H). LC-MS (Method 2): Rₜ = 1.13 min; MS (ESIpos): m/z = 495.5 [M + H]⁺ |
| 98 | 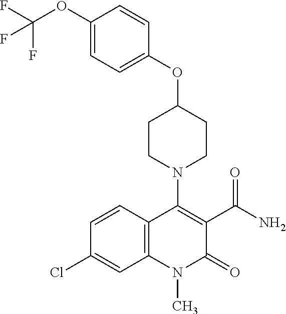 [SMILES: Cn1c(=O)c(C(N)=O)c(N2CCC(Oc3ccc(OC(F)(F)F)cc3)CC2)c2ccc(Cl)cc21] | 7-chloro-1- methyl-2-oxo- 4-{4-[4- (trifluorometho xy)phenoxy] piperidin-1-yl}-1,2- dihydroquinoline-3- carboxamide | 7-chloro-1- methyl-2-oxo- 4-{4-[4- (trifluoromethoxy) phenoxy] piperidin-1- yl}-1,2- dihydroquinoline- 3-carbonitrile (example 64) | ¹H NMR (DMSO-d₆) δ: 7.82-7.95 (m, 1H), 7.54- 7.61 (m, 1H), 7.22-7.38 (m, 3H), 7.04-7.11 (m, 2H), 4.51-4.71 (m, 1H), 3.54 (s, 3H), 3.28-3.41 (m, 2H), 3.11 (br t, 2H), 2.04- 2.22 (m, 2H), 1.74-1.97 (m, 2H). LC-MS (Method 2): Rₜ = 1.37 min; MS (ESIpos): m/z = 496.4 [M + H]⁺ |
| 99 | 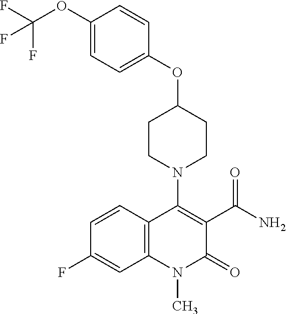 [SMILES: Cn1c(=O)c(C(N)=O)c(N2CCC(Oc3ccc(OC(F)(F)F)cc3)CC2)c2ccc(F)cc21] | 7-fluoro-1- methyl-2-oxo- 4-{4-[4- (trifluoromethoxy) phenoxy] piperidin-1-yl}-1,2- dihydroquinoline-3- carboxamide | 7-fluoro-1- methyl-2-oxo- 4-{4-[4- (trifluorometh oxy)phenoxy] piperidin-1- yl}-1,2- dihydroquinoline- 3-carbonitrile (example 63) | ¹H NMR (DMSO-d₆) δ: 7.94 (dd, 1H), 7.68 (s, 1H), 7.45-7.54 (m, 1H), 7.35- 7.42 (m, 1H), 7.29 (d, 2H), 7.05-7.21 (m, 3H), 4.62 (dt, 1H), 3.55 (s, 3H), 3.35-3.43 (m, 2H), 3.08- 3.22 (m, 2H), 2.07-2.19 (m, 2H), 1.77-1.92 (m, 2H). LC-MS (Method 2): Rₜ = 1.30 min; MS (ESIpos): m/z = 480.5 [M + H]⁺ |

### Example 100

### (rac)-1-methyl-2-oxo-4-{4-[4-(trifluoromethoxy)phenoxy]azepan-1-yl}-1,2-dihydroquinoline-3-carbonitrile

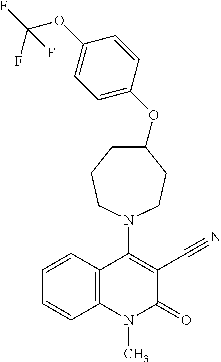 [SMILES: Cn1c(=O)c(C#N)c(N2CCCC(Oc3ccc(OC(F)(F)F)cc3)CC2)c2ccccc21]

A solution of 183 mg 4-chloro-1-methyl-2-oxo-1,2-dihydroquinoline-3-carbonitrile (796 μmol, CAS 150617-68-8, synthesis described in WO2012009649, example 1—compound III), 300 mg (rac)-4-[4-(trifluoromethoxy)phenoxy]azepane (1.04 mmol, intermediate 45) and 0.22 mL triethylamine (1.6 mmol) in 8 mL 2-propanol was stirred for 7 h at 90° C. After this time, water was added and the reaction was extracted with ethyl acetate. The organic phase was washed with water and brine and dried over sodium sulfate. After evaporation of the solvent, the residue was purified by flash chromatography (silica, dichloromethane/methanol gradient 0-3%). 192 mg of the title compound were obtained (95% purity, 50% yield).

¹H NMR (DMSO-d₆) δ: 7.98-8.02 (m, 1H), 7.69-7.78 (m, 1H), 7.55-7.60 (m, 1H), 7.24-7.39 (m, 3H), 7.05-7.12 (m, 2H), 4.74-4.84 (m, 1H), 3.64-3.84 (m, 4H), 3.57 (s, 3H), 2.13-2.28 (m, 2H), 1.78-2.08 (m, 4H).

LC-MS (Method 2): Rₜ=1.41 min; MS (ESIpos): m/z=458.5 [M+H]⁺

The title compound (192 mg) was separated into enantiomers by preparative chiral HPLC to give enantiomer 1 (50 mg, see example 102) and enantiomer 2 (55 mg, see example 101).

### Preparative Chiral HPLC Method

Instrument: Labomatic HD5000, Labocord-5000; Gilson GX-241, Labcol Vario 4000; column: Chiralpak IG 5μ 250×30 mm; eluent A: methanol; isocratic 100% A; flow 50.0 ml/min; UV 254 nm

### Analytical Chiral HPLC Method

Instrument: Agilent HPLC 1260; column: Chiralpak IG 3μ 100×4.6 mm; eluent A: methanol; isocratic 100% A; flow 1.4 ml/min; temperature: 25° C.; DAD 254 nm

### Example 101

### 1-methyl-2-oxo-4-{4-[4-(trifluoromethoxy)phenoxy]azepan-1-yl}-1,2-dihydroquinoline-3-carbonitrile, enantiomer 1

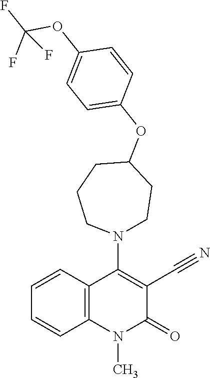 [SMILES: Cn1c(=O)c(C#N)c(N2CCCC(Oc3ccc(OC(F)(F)F)cc3)CC2)c2ccccc21]

For the preparation of the racemic title compound see example 100. Separation of enantiomers by preparative chiral HPLC (method see example 100) to give 55 mg of the title compound (99% purity, 15% yield).

Analytical chiral HPLC (method see example 100): Rₜ=3.41 min.

Optical rotation:[α]_{D}=+14.5° (c=10 mg/ml, methanol)

¹H NMR (DMSO-d₆) δ: 7.96-8.04 (m, 1H), 7.68-7.77 (m, 1H), 7.55-7.62 (m, 1H), 7.24-7.40 (m, 3H), 7.04-7.12 (m, 2H), 4.75-4.84 (m, 1H), 3.63-3.88 (m, 4H), 3.57 (s, 3H), 1.78-2.28 (m, 6H).

### Example 102

### 1-methyl-2-oxo-4-{4-[4-(trifluoromethoxy)phenoxy]azepan-1-yl}-1,2-dihydroquinoline-3-carbonitrile, enantiomer 2

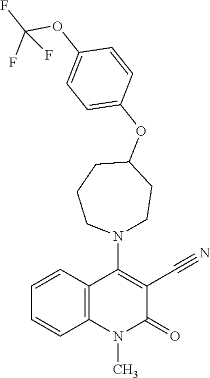 [SMILES: Cn1c(=O)c(C#N)c(N2CCCC(Oc3ccc(OC(F)(F)F)cc3)CC2)c2ccccc21]

For the preparation of the racemic title compound see example 100. Separation of enantiomers by preparative chiral HPLC (method see example 100) to give 50 mg of the title compound (99% purity, 14% yield).

Analytical chiral HPLC (method see example 100): Rₜ=2.85 min.

Optical rotation:[α]_{D}=−14.7°(c=10 mg/ml, methanol)

¹H NMR (DMSO-d₆) δ: 7.96-8.04 (m, 1H), 7.68-7.81 (m, 1H), 7.52-7.66 (m, 1H), 7.25-7.43 (m, 3H), 7.04-7.15 (m, 2H), 4.74-4.86 (m, 1H), 3.63-3.90 (m, 4H), 3.58 (s, 3H), 1.78-2.28 (m, 6H).

### Example 103

### (rac)-4-[4-(4-bromophenoxy)azepan-1-yl]-1-methyl-2-oxo-1,2-dihydroquinoline-3-carbonitrile

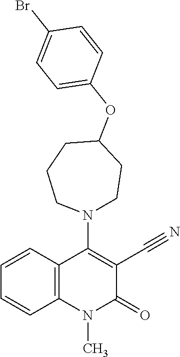 [SMILES: Cn1c(=O)c(C#N)c(N2CCCC(Oc3ccc(Br)cc3)CC2)c2ccccc21]

A solution of 212 mg 4-chloro-1-methyl-2-oxo-1,2-dihydroquinoline-3-carbonitrile (796 μmol, CAS 150617-68-8, synthesis described in WO2012009649, example 1—compound III), 486 mg (rac)-4-(4-bromophenoxy)azepane (1.26 mmol, intermediate 47) and 0.27 mL triethylamine (1.9 mmol) in 6.3 mL 2-propanol was stirred for 4 h at 90° C. After this time, water was added and the reaction was extracted with ethyl acetate. The organic phase was washed with water and brine and dried over sodium sulfate. After evaporation of the solvent, the residue was purified by flash chromatography (silica, dichloromethane/methanol gradient 0-3%). The crude product was purified by RP-HPLC (column: X-Bridge C18 5 μm 100×30 mm, mobile phase: acetonitrile/water (0.2 vol. % ammonia 32%)-gradient). 75 mg of the title compound were obtained (95% purity, 16% yield).

¹H NMR (DMSO-d₆) δ: 7.92-8.07 (m, 1H), 7.70-7.80 (m, 1H), 7.54-7.64 (m, 1H), 7.41-7.50 (m, 2H), 7.30-7.38 (m, 1H), 6.89-7.05 (m, 2H), 4.72-4.82 (m, 1H), 3.61-3.87 (m, 4H), 3.57 (s, 3H), 1.74-2.27 (m, 6H).

LC-MS (Method 2): Rₜ=1.42 min; MS (ESIpos): m/z=453.5 [M+H]⁺

### Example 104

### (rac)-4-{4-[4-(azetidin-1-yl)phenoxy]azepan-1-yl}-1-methyl-2-oxo-1,2-dihydroquinoline-3-carbonitrile

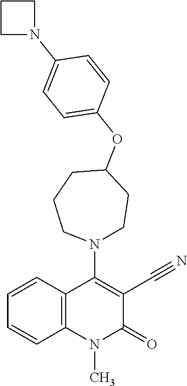 [SMILES: Cn1c(=O)c(C#N)c(N2CCCC(Oc3ccc(N4CCC4)cc3)CC2)c2ccccc21]

A mixture of 65 mg (rac)-4-[4-(4-bromophenoxy)azepan-1-yl]-1-methyl-2-oxo-1,2-dihydroquinoline-3-carbonitrile (137 μmol, example 103), 9.4 mg azetidine (164 μmol, CAS 503-29-7), 89 mg cesium carbonate (273 μmol) and 21.4 mg chloro(2-dicyclohexylphosphino-2′,4′,6′-triisopropyl-1,1′-biphenyl)[2-(2′-amino-1,1′-biphenyl)]palladium(II) (27.4 μmol, CAS 1310584-14-5) in 400 μL 1,4-dioxane was stirred for 16 h at 110° C. Water was added and the reaction was extracted with ethyl acetate (3×). The combined organic phases were washed with water and brine, dried and concentrated under reduced pressure. The residue was purified by flash chromatography (silica, dichloromethane/methanol gradient 0-3%) to give 10 mg of the title compound (95% purity, 16% yield).

¹H NMR (DMSO-d₆) δ: 7.97-8.04 (m, 1H), 7.69-7.78 (m, 1H), 7.53-7.62 (m, 1H), 7.30-7.39 (m, 1H), 6.81-6.88 (m, 2H), 6.30-6.42 (m, 2H), 4.47-4.62 (m, 1H), 3.78-3.88 (m, 1H), 3.61-3.78 (m, 7H), 3.55-3.60 (m, 3H), 2.21-2.29 (m, 2H), 1.76-2.19 (m, 6H).

LC-MS (Method 2): Rₜ=1.33 min; MS (ESIpos): m/z=229.6 [M+H]⁺

### Example 105

### (rac)-1-methyl-4-{4-[4-(1-methyl-1H-pyrazol-4-yl)phenoxy]azepan-1-yl}-2-oxo-1,2-dihydroquinoline-3-carbonitrile

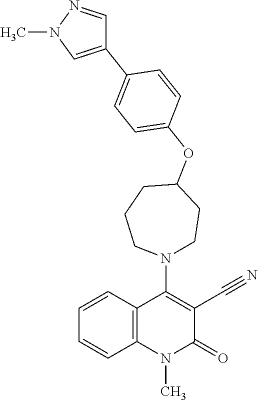 [SMILES: Cn1cc(-c2ccc(OC3CCCN(c4c(C#N)c(=O)n(C)c5ccccc45)CC3)cc2)cn1]

A mixture of 180 mg (rac)-4-[4-(4-bromophenoxy)azepan-1-yl]-1-methyl-2-oxo-1,2-dihydro-quinoline-3-carbonitrile (387 μmol, example 103), 60 mg (1-methyl-1H-pyrazol-4-yl)boronic acid (454 μmol, CAS 847818-55-7), 185 mg cesium carbonate (567 μmol), 30 mg chloro(2-dicyclohexylphosphino-2′,4′,6′-triisopropyl-1,1′-biphenyl)[2-(2′-amino-1,1′-biphenyl)]palladium(II) (38 μmol, CAS 1310584-14-5) and 22 mg palladium-triphenylphosphine (1:4) (19 μmol, CAS 14221-01-3) in 3 mL 1,4-dioxane was stirred for 3 h at 110° C. Water was added and the reaction was extracted with ethyl acetate (3×). The combined organic phases were washed with brine, dried and concentrated under reduced pressure. The residue was purified by flash chromatography (silica, dichloromethane/methanol gradient 0-3%) to give 42 mg of the title compound (95% purity, 23% yield).

¹H NMR (DMSO-d₆) δ: 7.95-8.06 (m, 2H), 7.70-7.77 (m, 2H), 7.52-7.59 (m, 1H), 7.44-7.51 (m, 2H), 7.30-7.38 (m, 1H), 6.94-7.03 (m, 2H), 4.71-4.82 (m, 1H), 3.79-3.91 (m, 4H), 3.63-3.76 (m, 3H), 3.58 (s, 3H), 2.11-2.27 (m, 2H), 1.92-2.08 (m, 3H), 1.77-1.90 (m, 1H).

LC-MS (Method 2): Rₜ=1.17 min; MS (ESIpos): m/z=454.6 [M+H]⁺

### Example 106

### 4-[4-(benzyloxy)piperidin-1-yl]-1-methyl-2-oxo-1,2-dihydroquinoline-3-carbonitrile

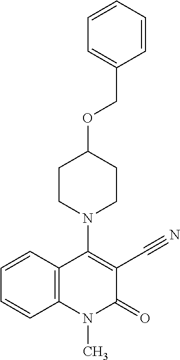 [SMILES: Cn1c(=O)c(C#N)c(N2CCC(OCc3ccccc3)CC2)c2ccccc21]

To a solution of 100 mg 4-(4-hydroxypiperidin-1-yl)-1-methyl-2-oxo-1,2-dihydroquinoline-3-carbonitrile (353 μmol, intermediate 1) and 46 μL (bromomethyl)benzene (390 μmol, CAS 100-39-0) in 2 mL DMF at 0° C. was added 28 mg sodium hydride (60% in mineral oil, 706 μmol) and the reaction was stirred for 2 h at rt. After this time, water was added and the mixture was stirred for some time. The residue was collected by filtration and washed with water. The resulting solid was dried under reduced pressure at 60° C. to give 106 mg of the title compound (77% yield, 96% purity).

1H-NMR (400 MHz, DMSO-d6): 5 [ppm]=1.75-1.88 (m, 2H), 2.12 (ddd, 2H), 3.40-3.49 (m, 2H), 3.56 (s, 3H), 3.68-3.81 (m, 3H), 4.59 (s, 2H), 7.24-7.42 (m, 6H), 7.56 (d, 1H), 7.73 (ddd, 1H), 7.85 (dd, 1H).

LC-MS (Method 2): Rₜ=1.26 min; MS (ESIpos): m/z=374.2 [M+H]⁺

### Example 107

### 1-methyl-4-{4-[(4-methylphenyl)methoxy]piperidin-1-yl}-2-oxo-1,2-dihydroquinoline-3-carbonitrile

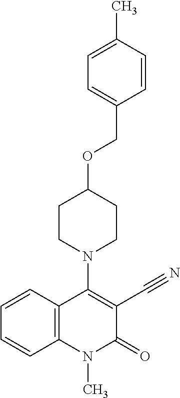 [SMILES: Cc1ccc(COC2CCN(c3c(C#N)c(=O)n(C)c4ccccc34)CC2)cc1]

To a solution of 100 mg 4-(4-hydroxypiperidin-1-yl)-1-methyl-2-oxo-1,2-dihydroquinoline-3-carbonitrile (353 μmol, intermediate 1) and 62 μL 1-(bromomethyl)-4-methylbenzene (460 μmol, CAS 104-81-4) in 2 mL DMF at 0° C. was added 28 mg sodium hydride (60% in mineral oil, 706 μmol) and the reaction was stirred for 2 h at rt. After this time, water was added and the mixture was stirred for some time. The residue was collected by filtration and washed with water. The resulting solid was dried under reduced pressure at 60° C. to give 114 mg of the title compound (82% yield, 98% purity).

1H-NMR (400 MHz, DMSO-d6): 5 [ppm]=1.72-1.91 (m, 2H), 2.10 (ddd, 2H), 2.30 (s, 3H), 3.43 (ddd, 2H), 3.56 (s, 3H), 3.72 (br dd, 3H), 4.54 (s, 2H), 7.17 (d, 2H), 7.23-7.29 (m, 2H), 7.30-7.39 (m, 1H), 7.55 (d, 1H), 7.69-7.76 (m, 1H), 7.84 (dd, 1H).

LC-MS (Method 2): Rₜ=1.34 min; MS (ESIpos): m/z=388.2 [M+H]⁺

### Example 108

### 1-methyl-4-{4-[(3-methylphenyl)methoxy]piperidin-1-yl}-2-oxo-1,2-dihydroquinoline-3-carbonitrile

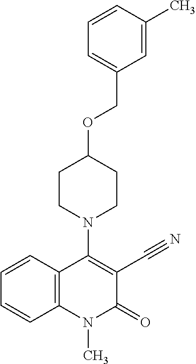 [SMILES: Cc1cccc(COC2CCN(c3c(C#N)c(=O)n(C)c4ccccc34)CC2)c1]

To a solution of 100 mg 4-(4-hydroxypiperidin-1-yl)-1-methyl-2-oxo-1,2-dihydroquinoline-3-carbonitrile (353 μmol, intermediate 1) and 52 μL 1-(bromomethyl)-3-methylbenzene 390 μmol, CAS 620-13-3) in 2 mL DMF at 0° C. was added 28 mg sodium hydride (60% in mineral oil, 706 μmol) and the reaction was stirred for 2 h at rt. After this time, water was added and the mixture was extracted with ethyl acetate (3×). The combined organic layers were filtered and concentrated under reduced pressure. 123 mg of the title compound were obtained (86% yield, 96% purity).

1H-NMR (400 MHz, DMSO-d6): 5 [ppm]=1.73-1.89 (m, 2H), 2.11 (ddd, 2H), 2.28-2.35 (m, 3H), 3.44 (ddd, 2H), 3.56 (s, 3H), 3.67-3.80 (m, 3H), 4.55 (s, 2H), 7.10 (d, 1H), 7.14-7.21 (m, 2H), 7.22-7.29 (m, 1H), 7.33 (ddd, 1H), 7.56 (dd, 1H), 7.73 (ddd, 1H), 7.85 (dd, 1H).

LC-MS (Method 2): Rₜ=1.35 min; MS (ESIpos): m/z=388.2 [M+H]⁺

TABLE 5
synthetic precedure for compound in analogy to example 108.

| Example | Structure | IUPAC-Name | Starting Materials | Analytics |
|---|---|---|---|---|
| 109 | 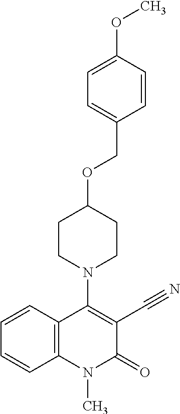 [SMILES: COc1ccc(COC2CCN(c3c(C#N)c(=O)n(C)c4ccccc34)CC2)cc1] | 4-{4-[(4- methoxyphenyl)m ethoxy]piperidin-1- yl}-1-methyl-2- oxo-1,2- dihydroquinoline- 3-carbonitrile | 4-(4-hydroxypiperidin- 1-yl)-1-methyl-2-oxo- 1,2-dihydroquinoline- 3-carbonitrile (Intermediate 1) and 1-(chloromethyl)-4- methoxybenzene (CAS 824-94-2) | ¹H NMR (DMSO-d₆) δ: 7.79-7.89 (m, 1H), 7.68-7.79 (m, 1H), 7.50-7.62 (m, 1H), 7.26-7.38 (m, 3H), 6.87-6.94 (m, 2H), 4.46-4.54 (m, 2H), 3.68-3.77 (m, 6H), 3.54-3.58 (m, 2H), 3.54-3.59 (m, 1H), 3.37-3.50 (m, 2H), 2.05-2.26 (m, 2H), 1.73-1.86 (m, 2H). |
| 110 | 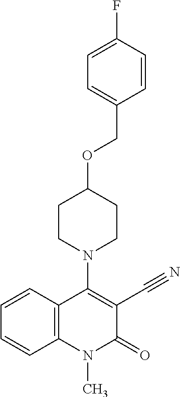 [SMILES: Cn1c(=O)c(C#N)c(N2CCC(OCc3ccc(F)cc3)CC2)c2ccccc21] | 4-{4-[(4- fluorophenyl)methoxy] piperidin-1-yl}- 1-methyl-2-oxo- 1,2- dihydroquinoline- 3-carbonitrile | 4-(4-hydroxypiperidin- 1-yl)-1-methyl-2-oxo- 1,2-dihydroquinoline- 3-carbonitrile (Intermediate 1) and 1-(bromomethyl)-4- fluorobenzene (CAS 459-46-1) | ¹H-NMR (400 MHz, DMSO-d6) δ [ppm]: 1.73-1.88 (m, 2H), 2.11 (ddd, 2H), 3.40- 3.50 (m, 2H), 3.56 (s, 3H), 3.67-3.83 (m, 3H), 4.57 (s, 2H), 7.13-7.26 (m, 2H), 7.28-7.36 (m, 1H), 7.38-7.46 (m, 2H), 7.55 (d, 1H), 7.73 (ddd, 1H), 7.80- 7.90 (m, 1H). |
| 111 | 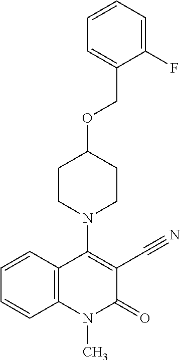 [SMILES: Cn1c(=O)c(C#N)c(N2CCC(OCc3ccccc3F)CC2)c2ccccc21] | 4-{4-[(2- fluorophenyl)methoxy] piperidin-1-yl}- 1-methyl-2-oxo- 1,2- dihydroquinoline- 3-carbonitrile | 4-(4-hydroxypiperidin- 1-yl)-1-methyl-2-oxo- 1,2-dihydroquinoline- 3-carbonitrile (Intermediate 1) and 1-(bromomethyl)-2- fluorobenzene (CAS 446-48-0) | ¹H-NMR (400 MHz, DMSO-d6) δ [ppm]: 1.73-1.89 (m, 2H), 2.06-2.20 (m, 2H), 3.45 (ddd, 2H), 3.56 (s, 3H), 3.66-3.87 (m, 3H),4.64 (s, 2H), 7.12-7.27 (m, 2H), 7.29-7.44 (m, 2H), 7.47-7.60 (m, 2H), 7.73 (ddd, 1H), 7.81-7.88 (m, 1H). |
| 112 | 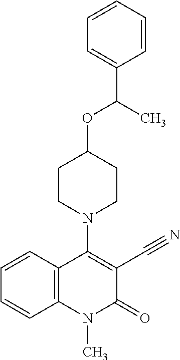 [SMILES: CC(OC1CCN(c2c(C#N)c(=O)n(C)c3ccccc23)CC1)c1ccccc1] | (rac)-1-methyl-2- oxo-4-[4-(1- phenylethoxy) piperidin-1-yl]-1,2- dihydroquinoline- 3-carbonitrile | 4-(4-hydroxypiperidin- 1-yl)-1-methyl-2-oxo- 1,2-dihydroquinoline- 3-carbonitrile (Intermediate 1) and (1- bromoethyl)benzene (CAS 585-71-7) | ¹H-NMR (400 MHz, DMSO-d6) δ [ppm]: 1.37 (d, 3H), 1.64- 1.75 (m, 1H), 1.75- 1.86 (m, 1H), 1.87- 1.95 (m, 1H), 2.06- 2.17 (m, 1H), 3.35- 3.46 (m, 2H), 3.50- 3.60 (m, 4H), 3.62- 3.78 (m, 2H), 4.72 (q, 1H), 7.23-7.43 (m, 6H), 7.55 (d, 1H), 7.72 (ddd, 1H), 7.82 (dd, 1H). |
| 113 | 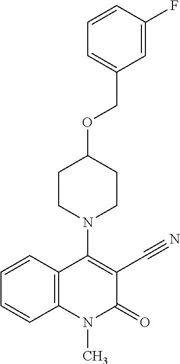 [SMILES: Cn1c(=O)c(C#N)c(N2CCC(OCc3cccc(F)c3)CC2)c2ccccc21] | 4-{4-[(3- fluorophenyl)methoxy] piperidin-1-yl}- 1-methyl-2-oxo- 1,2- dihydroquinoline- 3-carbonitrile | 4-(4-hydroxypiperidin- 1-yl)-1-methyl-2-oxo- 1,2-dihydroquinoline- 3-carbonitrile (Intermediate 1) and 1-(bromomethyl)-3- fluorobenzene (CAS 456-41-7) | ¹H-NMR (400 MHz, DMSO-d6) δ [ppm]: 7.85 (dd, 1 H), 7.73 (ddd, 1 H), 7.56 (dd, 1 H), 7.39- 7.45 (m, 1 H), 7.33 (ddd, 1 H), 7.17- 7.24(m, 2H), 7.07- 7.15 (m, 1 H), 4.62 (s, 2 H), 3.68- 3.83 (m, 3 H), 3.56 (s, 3 H), 3.45 (ddd, 2 H), 2.12 (ddd, 2 H), 1.76-1.91 (m, 2 H). |
| 114 | 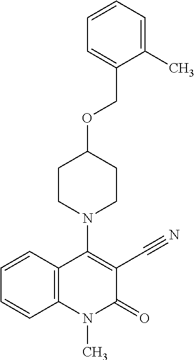 [SMILES: Cc1ccccc1COC1CCN(c2c(C#N)c(=O)n(C)c3ccccc23)CC1] | 1-methyl-4-{4-[(2- methylphenyl) methoxy]piperidin-1- yl}-2-oxo-1,2- dihydroquinoline- 3-carbonitrile | 4-(4-hydroxypiperidin- 1-yl)-1-methyl-2-oxo- 1,2-dihydroquinoline- 3-carbonitrile (Intermediate 1) and 1-(bromomethyl)-2- methylbenzene (CAS 89-92-9) | ¹H-NMR (400 MHz, DMSO-d6) δ [ppm]: 7.85 (dd, 1 H), 7.73 (ddd, 1 H), 7.56 (d, 1 H), 7.30-7.39 (m, 2 H), 7.15- 7.22 (m, 3 H), 4.57 (s, 2 H), 3.66-3.83 (m, 3 H), 3.56 (s, 3 H), 3.45 (ddd, 2 H), 2.32 (s, 3 H), 2.13 (ddd, 2H), 1.77- 1.92 (m, 2 H). |
| 115 | 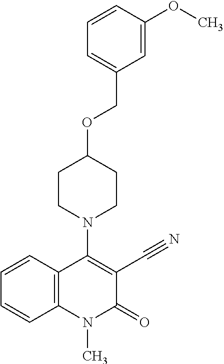 [SMILES: COc1cccc(COC2CCN(c3c(C#N)c(=O)n(C)c4ccccc34)CC2)c1] | 4-{4-[(3- methoxyphenyl) methoxy]piperidin-1- yl}-1-methyl-2- oxo-1,2- dihydroquinoline- 3-carbonitrile | 4-(4-hydroxypiperidin- 1-yl)-1-methyl-2-oxo- 1,2-dihydroquinoline- 3-carbonitrile (Intermediate 1) and 1-(bromomethyl)-3- methoxybenzene (CAS 874-98-6) | ¹H-NMR (400 MHz, DMSO-d6) δ [ppm]: 7.85 (dd, 1 H), 7.73 (ddd, 1 H), 7.56 (dd, 1 H), 7.33 (ddd, 1 H), 7.23- 7.30 (m, 1 H), 6.89- 6.97 (m, 2H), 6.81-6.88 (m, 1 H), 4.57 (s, 2 H), 3.69-3.79 (m, 6 H), 3.56 (s, 3 H), 3.44 (ddd, 2 H), 2.11 (ddd, 2 H), 1.76-1.87 (m, 2 H). |

### Example 116

### 4-{4-[(4-chlorophenoxy)methyl]piperidin-1-yl}-1-methyl-2-oxo-1,2-dihydroquinoline-3-carbonitrile

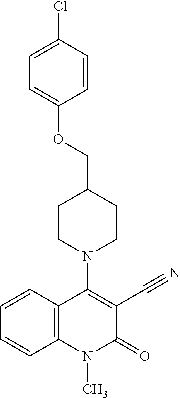 [SMILES: Cn1c(=O)c(C#N)c(N2CCC(COc3ccc(Cl)cc3)CC2)c2ccccc21]

To a suspension of 100 mg 4-[4-(hydroxymethyl)piperidin-1-yl]-1-methyl-2-oxo-1,2-dihydroquinoline-3-carbonitrile (336 μmol, intermediate 50), 82 mg 4-chlorophenol (639 μmol, CAS 106-48-9) and 176 mg triphenylphosphine (673 μmol) in 3.8 mL THF was added 130 μL diisopropyl azodicarboxylate (670 μmol). The reaction was stirred for 24 h at rt. The mixture was concentrated under reduced pressure. The residue was stirred in DMSO, the precipitate was collected by filtration and dried in vacuum. 63 mg of the title compound were obtained (44% yield, 95% purity).

¹H NMR (DMSO-d₆) δ: 7.78-7.90 (m, 1H), 7.66-7.77 (m, 1H), 7.51-7.61 (m, 1H), 7.30-7.38 (m, 3H), 6.94-7.07 (m, 2H), 3.92-3.98 (m, 2H), 3.76-3.86 (m, 2H), 3.58 (s, 3H), 3.39-3.48 (m, 2H), 2.05-2.22 (m, 1H), 1.90-2.00 (m, 2H), 1.54-1.69 (m, 2H).

LC-MS (Method 1): Rₜ=1.43 min; MS (ESIpos): m/z=408 [M+H]⁺

TABLE 6
examples prepared.

| | | | Starting | |
|---|---|---|---|---|
| | | | Materials, | |
| | | | procedure from | |
| Example | Structure | IUPAC-Name | example | Analytics |
| 117 | 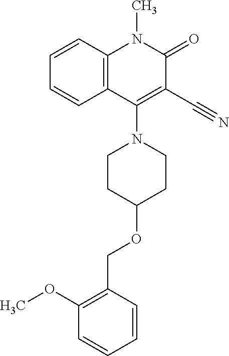 [SMILES: COc1ccccc1COC1CCN(c2c(C#N)c(=O)n(C)c3ccccc23)CC1] | 4-{4-[(2- methoxyphenyl) methoxy]piperidin-1- yl}-1-methyl-2- oxo-1,2- dihydroquinoline- 3-carbonitrile | In analogy to example 95 with 4- chloro-1-methyl-2- oxo-1,2- dihydroquinoline-3- carbonitrile (CAS 150617-68-8) and 4-[(2- methoxybenzyl)oxy] piperidine (CAS 86928-12-3) and with 1- methylpyrrolidin-2- one as solvent | |
| 118 | 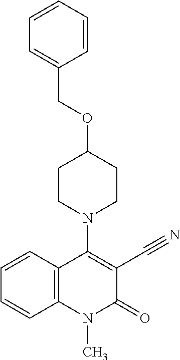 [SMILES: Cn1c(=O)c(C#N)c(N2CCC(OCc3ccccc3)CC2)c2ccccc21] | 1-methyl-2-oxo-4- [4- (phenoxymethyl) piperidin-1-yl]-1,2- dihydroquinoline- 3-carbonitrile | in analogy to example 116 with 4- [4- (hydroxymethyl) piperidin-1-yl]-1- methyl-2-oxo-1,2- dihydroquinoline-3- carbonitrile (intermediate 50) and phenol (CAS 108-95-2) | ¹H NMR (DMSO-d₆) δ: 7.83-7.91 (m, 1H), 7.68-7.78 (m, 1H), 7.51-7.61 (m, 1H), 7.24-7.38 (m, 3H), 6.89-7.02 (m, 3H), 3.89-3.98 (m, 2H), 3.75-3.85 (m, 2H), 3.58 (s, 3H), 3.39- 3.49 (m, 2H), 2.07- 2.25 (m, 1H), 1.93- 2.03 (m, 2H), 1.55- 1.72 (m, 2H). LC-MS (Method 1): Rₜ = 1.35 min; MS (ESIpos): m/z = 374.4 [M + H]⁺ |
| 119 | 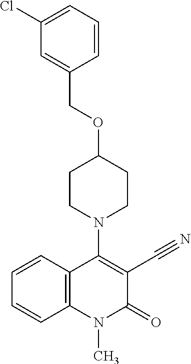 [SMILES: Cn1c(=O)c(C#N)c(N2CCC(OCc3cccc(Cl)c3)CC2)c2ccccc21] | 4-{4-[(3- chlorophenoxy) methyl]piperidin-1-yl}- 1-methyl-2-oxo- 1,2- dihydroquinoline- 3-carbonitrile | in analogy to example 116 with 4- [4- (hydroxymethyl) piperidin-1-yl]-1- methyl-2-oxo-1,2- dihydroquinoline-3- carbonitrile (intermediate 50) and 3-chlorophenol (CAS 108-43-0) | ¹H NMR (DMSO-d₆) δ: 7.80-7.90 (m, 1H), 7.69-7.78 (m, 1H), 7.50-7.60 (m, 1H), 7.22-7.42 (m, 2H), 7.02-7.10 (m, 1H), 6.92-7.01 (m, 2H), 3.94-4.03 (m, 2H), 3.75-3.86 (m, 2H), 3.57 (s, 3H), 3.37- 3.49 (m, 2H), 2.07- 2.21 (m, 1H), 1.88- 2.03 (m, 2H), 1.54- 1.71 (m, 2H). LC-MS (Method 1): Rₜ = 1.44 min; MS (ESIpos): m/z = 408 [M + H]⁺ |
| 120 | 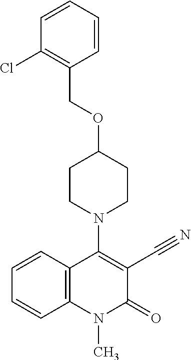 [SMILES: Cn1c(=O)c(C#N)c(N2CCC(OCc3ccccc3Cl)CC2)c2ccccc21] | 4-{4-[(2- chlorophenoxy) methyl]piperidin-1-yl}- 1-methyl-2-oxo- 1,2- dihydroquinoline- 3-carbonitrile | in analogy to example 116 with 4- [4- (hydroxymethyl) piperidin-1-yl]-1- methyl-2-oxo-1,2- dihydroquinoline-3- carbonitrile (intermediate 50) and 2-chlorophenol (CAS 95-57-8) | ¹H NMR (DMSO-d₆) δ: 7.85 (dd, J = 8.4, 1.3 Hz, 1H), 7.70-7.77 (m, 1H), 7.55-7.59 (m, 1H), 7.41-7.47 (m, 1H), 7.28-7.37 (m, 2H), 7.14-7.22 (m, 1H), 6.89-7.04 (m, 1H), 4.04 (d, J = 6.3 Hz, 2H), 3.82 (br d, J = 12.7 Hz, 2H), 3.57 (s, 3H), 3.40- 3.51 (m, 2H), 2.12- 2.26 (m, 1H), 2.00 (br dd, J = 10.8, 2.2 Hz, 2H), 1.59-1.76 (m, 2H). LC-MS (Method 1): Rₜ = 1.40 min; MS (ESIpos): m/z = 408 [M + H]⁺ |

### Example 121

### 4-[4-(benzyloxy)piperidin-1-yl]-1-methyl-2-oxo-1,2-dihydroquinoline-3-carboxamide

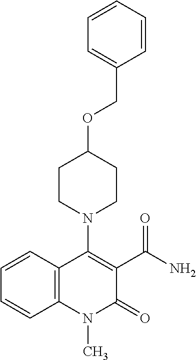 [SMILES: Cn1c(=O)c(C(N)=O)c(N2CCC(OCc3ccccc3)CC2)c2ccccc21]

A solution of 70 mg 4-[4-(benzyloxy)piperidin-1-yl]-1-methyl-2-oxo-1,2-dihydroquinoline-3-carbonitrile (189 μmol, example 106), 11 mg palladium(II)diacetate (47 μmol) and 56 mg N-[(1E)-ethylidene]hydroxylamine (943 μmol, CAS 107-29-9) in 2 mL ethanol was stirred for 3 h at 80° C. Water was added and the reaction was extracted with ethyl acetate (3×). The combined organic layers were filtered and concentrated under reduced pressure. The crude was purified by RP-HPLC (column: X-Bridge C18 5 μm 100×30 mm, mobile phase: acetonitrile/water (0.2 vol. % ammonia 32%)-gradient) to give 33.5 mg of the title compound (100% purity, 45% yield).

1H NMR (400 MHz, DMSO-d6) δ ppm 7.89 (dd, 1H), 7.58-7.68 (m, 2H), 7.51 (dd, 1H), 7.44 (d, 1H), 7.36 (d, 4H), 7.26-7.33 (m, 2H), 4.57 (s, 2H), 3.57 (s, 4H), 3.26-3.40 (m, 2H), 3.03 (ddd, 2H), 1.98-2.11 (m, 2H), 1.65-1.80 (m, 2H).

LC-MS (Method 3): Rₜ=1.20 min; MS (ESIpos): m/z=392 [M+H]⁺

### Example 122

### 1-methyl-4-{4-[(4-methylphenyl)methoxy]piperidin-1-yl}-2-oxo-1,2-dihydroquinoline-3-carboxamide

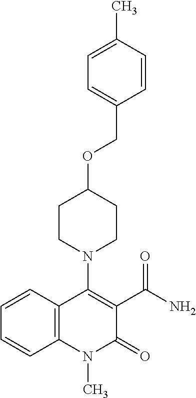 [SMILES: Cc1ccc(COC2CCN(c3c(C(N)=O)c(=O)n(C)c4ccccc34)CC2)cc1]

A solution of 78 mg 1-methyl-4-{4-[(4-methylphenyl)methoxy]piperidin-1-yl}-2-oxo-1,2-dihydroquinoline-3-carbonitrile (201 μmol, example 107), 11 mg palladium(II)diacetate (50 μmol) and 59 mg N-[(1E)-ethylidene]hydroxylamine (1.0 mmol, CAS 107-29-9) in 2 mL ethanol was stirred for 17 h at 80° C. Water was added and the reaction was extracted with ethyl acetate (3×). The combined organic layers were filtered and concentrated under reduced pressure. The crude was purified by RP-HPLC (column: X-Bridge C18 5 μm 100×30 mm, mobile phase: acetonitrile/water (0.2 vol. % ammonia 32%)-gradient) to give 10.5 mg of the title compound (100% purity, 13% yield).

1H NMR (400 MHz, DMSO-d6) δ ppm 7.88 (dd, 1H), 7.58-7.67 (m, 2H), 7.51 (d, 1H), 7.44 (br d, 1H), 7.27-7.34 (m, 1H), 7.22-7.27 (m, 2H), 7.12-7.19 (m, 2H), 4.51 (s, 2H), 3.53-3.59 (m, 4H), 3.30-3.41 (m, 2H), 2.97-3.07 (m, 2H), 2.30 (s, 3H), 1.97-2.07 (m, 2H), 1.65-1.78 (m, 2H).

LC-MS (Method 3): Rₜ=1.29 min; MS (ESIpos): m/z=406 [M+H]⁺

TABLE 7
in analogy to example 122

| | | | Starting | |
|---|---|---|---|---|
| Example | Structure | IUPAC-Name | Materials | Analytics |
| 123 | 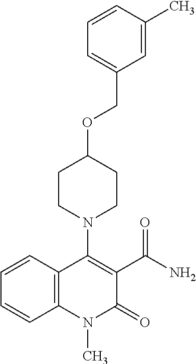 [SMILES: Cc1cccc(COC2CCN(c3c(C(N)=O)c(=O)n(C)c4ccccc34)CC2)c1] | 1-methyl-4-{4-[(3- methylphenyl) methoxy]piperidin-1- yl}-2-oxo-1,2- dihydroquinoline- 3-carboxamide | 1-methyl-4-{4- [(3- methylbenzyl) oxy]piperidin-1-yl}- 2-oxo-1,2- dihydroquinoline- 3-carbonitrile (example 108) | ¹H-NMR (400 MHz, DMSO-d6) δ [ppm]: 7.89 (dd, 1 H), 7.59-7.68 (m, 2 H), 7.51 (dd, 1 H), 7.44 (br d, 1 H), 7.27-7.32 (m, 1 H), 7.22-7.27 (m, 1 H), 7.12-7.19 (m, 2 H), 7.09 (d, 1 H), 4.52 (s, 2 H), 3.57 (s, 4 H), 3.28- 3.36 (m, 2 H), 2.93-3.12 (m, 2 H), 2.31 (s, 3 H), 1.97-2.11 (m, 2 H), 1.66- 1.84 (m, 2 H). LC-MS (Method 2): Rₜ = 1.16 min; MS (ESIpos): m/z = 406.3 [M + H]⁺ |
| 124 | 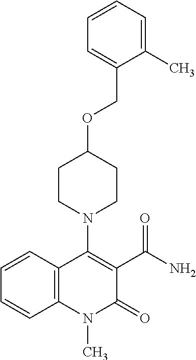 [SMILES: Cc1ccccc1COC1CCN(c2c(C(N)=O)c(=O)n(C)c3ccccc23)CC1] | 1-methyl-4-{4-[(2- methylphenyl) methoxy]piperidin-1- yl}-2-oxo-1,2- dihydroquinoline- 3-carboxamide | 1-methyl-4-{4- 1(2- methylbenzyl) oxy]piperidin-1-yl}- 2-oxo-1,2- dihydroquinoline- 3-carbonitrile (example 114) | ¹H NMR (DMSO-d₆) δ: 7.89 (dd, 1H), 7.59-7.67 (m, 2H), 7.51 (dd, J = 8.6, 0.8 Hz, 1H), 7.42-7.47 (m, 1H), 7.33-7.37 (m, 1H), 7.27-7.32 (m, 1H), 7.15-7.22 (m, 3H), 4.49- 4.62 (m, 2H), 3.52-3.64 (m, 4H), 3.34-3.37 (m, 1H), 3.32 (brs, 1H), 2.96-3.09 (m, 2H), 2.27- 2.35 (m, 3H), 1.97-2.10 (m, 2H), 1.66-1.82 (m, 2H). LC-MS (Method 2): Rₜ = 1.16 min; MS (ESIpos): m/z = 406.3 [M + H]⁺ |
| 125 | 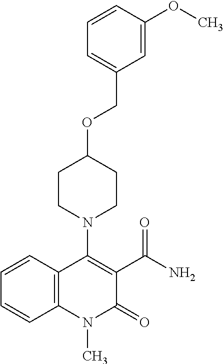 [SMILES: COc1cccc(COC2CCN(c3c(C(N)=O)c(=O)n(C)c4ccccc34)CC2)c1] | 4-{4-[(3- methoxyphenyl) methoxy]piperidin-1- yl}-1-methyl-2- oxo-1,2- dihydroquinoline- 3-carboxamide | 4-{4-[(3- methoxybenzyl) oxy]piperidin-1- yl}-1-methyl-2- oxo-1,2- dihydroquinoline- 3-carbonitrile (example 115) | ¹H-NMR (400 MHz, DMSO-d6) δ [ppm]: 7.89 (dd, 1 H), 7.57-7.68 (m, 2 H), 7.48-7.56 (m, 1 H), 7.44 (d, 1 H), 7.21- 7.34 (m, 2 H), 6.90-6.98 (m, 2 H), 6.85 (dt, 1 H), 4.54 (s, 2 H), 3.76 (s, 3 H), 3.57 (s, 4 H), 3.35 (br s, 1 H), 3.32 (brs, 1 H), 2.94-3.10 (m, 2 H), 1.94- 2.12 (m, 2 H), 1.62- 1.85 (m, 2 H). LC-MS (Method 2): Rₜ = 1.08 min; MS (ESIpos): m/z = 422.3 [M + H]⁺ |
| 126 | 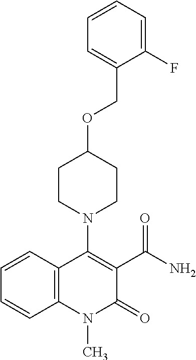 [SMILES: Cn1c(=O)c(C(N)=O)c(N2CCC(OCc3ccccc3F)CC2)c2ccccc21] | 4-{4-[(2- fluorophenyl)methoxy] piperidin-1-yl}- 1-methyl-2-oxo- 1,2- dihydroquinoline- 3-carboxamide | 4-{4-[(2- fluorobenzyl)oxy] piperidin-1-yl}- 1-methyl-2-oxo- 1,2- dihydroquinoline- 3-carbonitrile (example 111) | ¹H NMR (DMSO-d₆) δ: 7.85-7.91 (m, 1H), 7.58- 7.69 (m, 2H), 7.47-7.54 (m, 2H), 7.45 (d, 1H), 7.37 (tdd, 1H), 7.27-7.32 (m, 1H), 7.16-7.25 (m, 2H), 4.59-4.66 (m, 2H), 3.54-3.67 (m, 4H), 3.34- 3.37 (m, 1H), 3.29-3.32 (m, 1H), 2.99-3.10 (m, 2H), 2.00-2.11 (m, 2H), 1.68-1.82 (m, 2H). LC-MS (Method 2): Rₜ = 1.11 min; MS (ESIpos): m/z = 410.2 [M + H]⁺ |
| 127 | 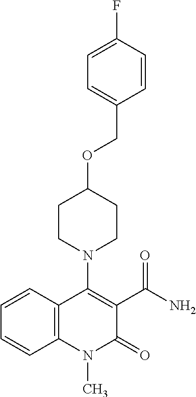 [SMILES: Cn1c(=O)c(C(N)=O)c(N2CCC(OCc3ccc(F)cc3)CC2)c2ccccc21] | 4-{4-[(4- fluorophenyl)methoxy] piperidin-1-yl}- 1-methyl-2-oxo- 1,2- dihydroquinoline- 3-carboxamide | 4-{4-[(4- fluorobenzyl)oxy] piperidin-1-yl}- 1-methyl-2-oxo- 1,2- dihydroquinoline- 3-carbonitrile (example 110) | ¹H-NMR (400 MHz, DMSO-d6) δ [ppm]: 7.88 (dd, 1 H), 7.58-7.67 (m, 2 H), 7.51 (dd, 1 H), 7.36- 7.46 (m,3H), 7.26- 7.34 (m, 1 H), 7.14-7.22 (m, 2 H), 4.55 (s, 2 H), 3.57 (s, 4 H), 3.34-3.38 (m, 1 H), 3.28-3.32 (m, 1 H), 3.03 (ddd, 2 H), 1.97-2.09 (m, 2 H), 1.65- 1.81 (m, 2 H). LC-MS (Method 2): Rₜ = 1.11 min; MS (ESIpos): m/z = 410.2 [M + H]⁺ |
| 128 | 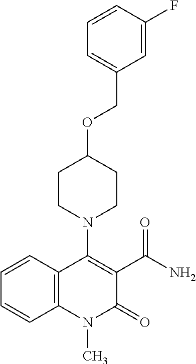 [SMILES: Cn1c(=O)c(C(N)=O)c(N2CCC(OCc3cccc(F)c3)CC2)c2ccccc21] | 4-{4-[(3- fluorophenyl)methoxy] piperidin-1-yl}- 1-methyl-2-oxo- 1,2- dihydroquinoline- 3-carboxamide | 4-{4-[(3- fluorobenzyl)oxy] piperidin-1-yl}- 1-methyl-2-oxo- 1,2- dihydroquinoline- 3-carbonitrile (example 113) | ¹H-NMR (400 MHz, DMSO-d6) δ [ppm]: 7.89 (dd, 1 H), 7.58-7.68 (m, 2 H), 7.49-7.56 (m, 1 H), 7.36-7.47 (m, 2 H), 7.30 (td, 1 H), 7.15-7.25 (m, 2 H), 7.06-7.14 (m, 1 H), 4.59 (s, 2 H), 3.54- 3.64 (m, 4 H), 3.35 (br s, 1 H), 3.27-3.32 (m, 1 H), 2.98-3.10 (m, 2 H), 1.98-2.11 (m, 2H), 1.68- 1.83 (m, 2 H). LC-MS (Method 2): Rₜ = 1.11 min; MS (ESIpos): m/z = 410.2 [M + H]⁺ |

### Example 129

### 8-fluoro-1-methyl-2-oxo-4-{4-[4-(trifluoromethoxy)phenoxy]piperidin-1-yl}-1,2-dihydroquinoline-3-carbonitrile

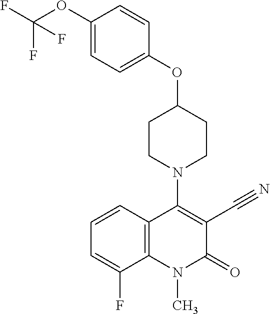 [SMILES: Cn1c(=O)c(C#N)c(N2CCC(Oc3ccc(OC(F)(F)F)cc3)CC2)c2cccc(F)c21]

A solution of 100 mg 4-chloro-8-fluoro-1-methyl-2-oxo-1,2-dihydroquinoline-3-carbonitrile (423 μmol, intermediate 53), 144 mg 4-[4-(trifluoromethoxy)phenoxy]piperidine (549 μmol, CAS 287952-67-4) and 0.12 mL triethylamine (850 μmol) in 2.8 mL 2-propanol was stirred for 3 h at 90° C. After this time, water was added and the reaction was extracted with ethyl acetate. The organic phase was washed with water and brine and dried over sodium sulfate. After evaporation of the solvent, the residue was purified by flash chromatography (silica, dichloromethane/methanol gradient 0-3%). 71 mg of the title compound were obtained (95% purity, 35% yield).

¹H NMR (DMSO-d₆) δ: 7.66-7.77 (m, 1H), 7.55-7.66 (m, 1H), 7.25-7.41 (m, 3H), 7.08-7.19 (m, 2H), 4.73-4.84 (m, 1H), 3.66-3.80 (m, 5H), 3.49-3.60 (m, 2H), 2.13-2.28 (m, 2H), 1.81-1.99 (m, 2H).

LC-MS (Method 2): Rₜ=1.44 min; MS (ESIpos): m/z=462.3 [M+H]⁺

### Example 130

### 8-bromo-1-methyl-2-oxo-4-{4-[4-(trifluoromethoxy)phenoxy]piperidin-1-yl}-1,2-dihydroquinoline-3-carbonitrile

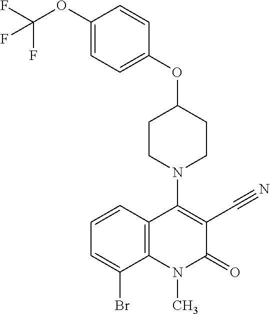 [SMILES: Cn1c(=O)c(C#N)c(N2CCC(Oc3ccc(OC(F)(F)F)cc3)CC2)c2cccc(Br)c21]

A suspension of 1.3 g 8-bromo-4-chloro-1-methyl-2-oxo-1,2-dihydroquinoline-3-carbonitrile (4.37 mmol, intermediate 56), 1.37 g 4-[4-(trifluoromethoxy)phenoxy]piperidine (5.24 mmol, CAS 287952-67-4) and 2.3 mL N,N-diisopropylethylamine (340 mmol) in 26 mL 2-propanol was stirred for 2 h at 90° C. After this time, water was added and the reaction was stirred for some time. The residue was collected by filtration and washed with ethanol. The resulting solid was dried to give 2.17 g of the title compound (90% yield, 95% purity).

¹H NMR (DMSO-d₆) δ: 7.94-8.07 (m, 1H), 7.81-7.90 (m, 1H), 7.20-7.34 (m, 3H), 7.09-7.19 (m, 2H), 4.71-4.87 (m, 1H), 3.64-3.82 (m, 5H), 3.49-3.62 (m, 2H), 2.14-2.26 (m, 2H), 1.84-1.99 (m, 2H).

LC-MS (Method 1): Rₜ=1.47 min; MS (ESIpos): m/z=524 [M+H]⁺

### Example 131

### 8-chloro-1-methyl-2-oxo-4-{4-[4-(trifluoromethoxy)phenoxy]piperidin-1-yl}-1,2-dihydroquinoline-3-carbonitrile

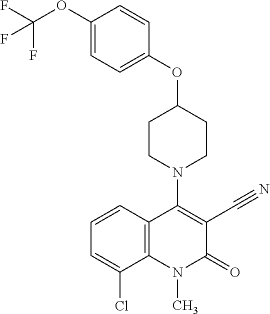 [SMILES: Cn1c(=O)c(C#N)c(N2CCC(Oc3ccc(OC(F)(F)F)cc3)CC2)c2cccc(Cl)c21]

A suspension of 120 mg 4,8-dichloro-1-methyl-2-oxo-1,2-dihydroquinoline-3-carbonitrile (446 μmol, intermediate 59), 144 mg 4-[4-(trifluoromethoxy)phenoxy]piperidine (535 μmol, CAS 287952-67-4) and 230 μL N,N-diisopropylethylamine (1.3 mmol) in 3 mL 2-propanol was stirred for 2 h at 90° C. After this time, water was added and the reaction was stirred for some time. The residue was collected by filtration and washed with water, ethanol and hexane. The resulting solid was dried to give 212 mg of the title compound (99% yield, 99% purity).

¹H NMR (DMSO-d₆) δ: 7.82 (ddd, 2H), 7.28-7.35 (m, 3H), 7.12-7.17 (m, 2H), 4.74-4.82 (m, 1H), 3.72-3.80 (m, 2H), 3.70 (s, 3H), 3.55 (ddd, 2H), 2.20 (ddd, 2H), 1.86-1.97 (m, 2H).

LC-MS (Method 1): Rₜ=1.49 min; MS (ESIpos): m/z=478.4 [M+H]⁺

### Example 132

### 1-methyl-2-oxo-4-{4-[4-(trifluoromethoxy)phenoxy]piperidin-1-yl}-1,2-dihydroquinoline-3,8-dicarbonitrile

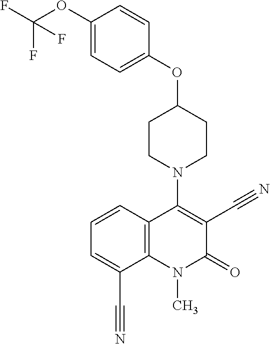 [SMILES: Cn1c(=O)c(C#N)c(N2CCC(Oc3ccc(OC(F)(F)F)cc3)CC2)c2cccc(C#N)c21]

To a solution of 120 mg 8-bromo-1-methyl-2-oxo-4-{4-[4-(trifluoromethoxy)phenoxy]piperidin-1-yl}-1,2-dihydroquinoline-3-carbonitrile (230 μmol, example 130) in 2.6 mL DMF was added 103 mg copper(I)cyanide (1.15 mmol, CAS 544-92-3) and the mixture was stirred for 12 h at 150° C. in the microwave. The mixture was cooled down to rt, a solution of 745 mg trichloroiron hexahydrate in 720 μL water and 120 μL hydrochloric acid (concentrated) was added and the mixture was stirred for 15 min. at 70° C. After cooled down to rt, water was added and the precipitate was collected by filtration and washed with ethanol. The resulting solid was dried to give 91 mg of the title compound (80% yield, 95% purity).

¹H NMR (DMSO-d₆) δ: 8.09-8.32 (m, 2H), 7.40-7.48 (m, 1H), 7.24-7.38 (m, 2H), 7.08-7.21 (m, 2H), 4.66-4.90 (m, 1H), 3.89 (s, 3H), 3.68-3.80 (m, 2H), 3.45-3.62 (m, 2H), 2.12-2.26 (m, 2H), 1.84-2.01 (m, 2H).

LC-MS (Method 1): Rₜ=1.39 min; MS (ESIpos): m/z=469 [M+H]⁺

### Example 133

### 1-methyl-2-oxo-8-(2-oxopyrrolidin-1-yl)-4-{4-[4-(trifluoromethoxy)phenoxy]piperidin-1-yl}-1,2-dihydroquinoline-3-carbonitrile

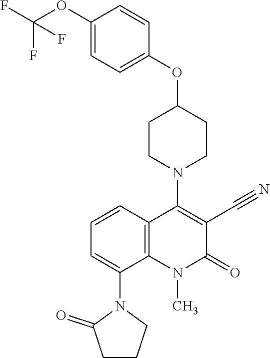 [SMILES: Cn1c(=O)c(C#N)c(N2CCC(Oc3ccc(OC(F)(F)F)cc3)CC2)c2cccc(N3CCCC3=O)c21]

A suspension of 100 mg 8-bromo-1-methyl-2-oxo-4-{4-[4-(trifluoromethoxy)phenoxy]piperidin-1-yl}-1,2-dihydroquinoline-3-carbonitrile (191 μmol, example 130), 32.6 mg pyrrolidin-2-one (383 μmol, CAS 616-45-5), 82 μL N,N′-dimethylethane-1,2-diamine (770 μmol), 7.3 mg copper(I)iodide (38 μmol) and 58 mg potassium carbonate (421 μmol) in 3.3 mL toluene was stirred overnight at 110° C. The mixture was cooled down to rt, water was added and the mixture was extracted with dichloromethane. The combined organic phases were washed with brine, filtered (using a waterresistant filter) and concentrated under reduced pressure. The residue was purified by flash chromatography (silica, hexane/ethyl acetate gradient 0-100%, dichloromethane/ethanol gradient 0-10%). The impure product was stirred in ethanol, the precipitate was collected by filtration and dried in vacuum. 30 mg of the title compound were obtained (28% yield, 95% purity).

¹H NMR (DMSO-d₆) δ: 7.78-7.89 (m, 1H), 7.63-7.71 (m, 1H), 7.24-7.41 (m, 3H), 7.10-7.19 (m, 2H), 4.71-4.91 (m, 1H), 3.84-3.93 (m, 1H), 3.68-3.82 (m, 3H), 3.48-3.63 (m, 2H), 3.35 (s, 3H), 2.43-2.47 (m, 2H), 2.12-2.28 (m, 4H), 1.85-2.00 (m, 2H).

LC-MS (Method 1): Rₜ=1.30 min; MS (ESIpos): m/z=528 [M+H]⁺

### Example 134

### 8-{[dimethyl(oxo)-λ⁶-sulfanylidene]amino}-1-methyl-2-oxo-4-{4-[4-(trifluoromethoxy)phenoxy]piperidin-1-yl}-1,2-dihydroquinoline-3-carbonitrile

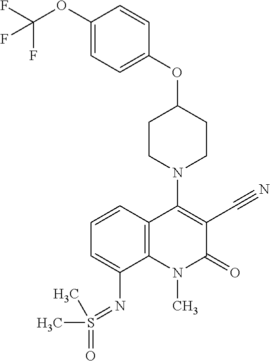 [SMILES: Cn1c(=O)c(C#N)c(N2CCC(Oc3ccc(OC(F)(F)F)cc3)CC2)c2cccc(N=S(C)(C)=O)c21]

To a solution of 100 mg 8-bromo-1-methyl-2-oxo-4-{4-[4-(trifluoromethoxy)phenoxy]piperidin-1-yl}-1,2-dihydroquinoline-3-carbonitrile (191 μmol, example 130) in 2.0 mL 1,4-dioxane was added 21.4 mg sulfonimidoyldimethane (230 μmol, CAS 1520-31-6), 12 mg tris(dibenzylideneacetone)dipalladium (13 μmol, CAS 51364-51-3), 11 mg biphenyl-2-yl(di-tert-butyl)phosphine (38 μmol, CAS 224311-51-7) and 26 mg sodium 2-methylpropan-2-olate (268 μmol). The reaction was stirred for 1.5 h at 80° C. in the microwave. The mixture was cooled down to rt and was concentrated under reduced pressure. To the residue was added water and the mixture was extracted with dichloromethane. The combined organic phases were washed with brine, filtered (using a waterresistant filter) and concentrated under reduced pressure. The residue was purified by RP-HPLC (column: X-Bridge C18 5 μm 100×30 mm, mobile phase: acetonitrile/water (0.1 vol. % formic acid)-gradient) to give 21 mg of the title compound (95% purity, 19% yield).

¹H NMR (DMSO-d₆) δ: 7.25-7.48 (m, 4H), 7.06-7.21 (m, 3H), 4.69-4.84 (m, 1H), 3.68-3.81 (m, 5H), 3.43-3.62 (m, 2H), 3.23 (s, 6H), 2.14-2.24 (m, 2H), 1.84-1.95 (m, 2H).

LC-MS (Method 1): Rₜ=1.33 min; MS (ESIpos): m/z=535 [M+H]⁺

### Example 135

### 8-(dimethylphosphoryl)-1-methyl-2-oxo-4-{4-[4-(trifluoromethoxy)phenoxy]piperidin-1-yl}-1,2-dihydroquinoline-3-carbonitrile

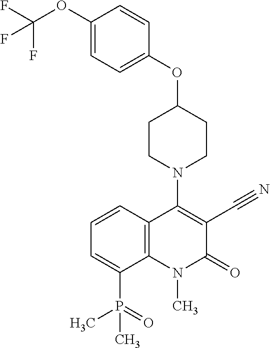 [SMILES: Cn1c(=O)c(C#N)c(N2CCC(Oc3ccc(OC(F)(F)F)cc3)CC2)c2cccc(P(C)(C)=O)c21]

To a mixture of 100 mg 8-bromo-1-methyl-2-oxo-4-{4-[4-(trifluoromethoxy)phenoxy]piperidin-1-yl}-1,2-dihydroquinoline-3-carbonitrile (191 μmol, example 130), 6.7 mg (9,9-dimethyl-9H-xanthene-4,5-diyl)bis(diphenylphosphine) (11.5 μmol), 2.2 mg palladium(II)diacetate (9.6 μmol) and 45 mg tripotassium phosphate (211 μmol) in 1.6 mL degassed DMF was added 18 mg dimethylphosphine oxide (211 μmol, CAS 7211-39-4) and the reaction was stirred for 40 min. at 130° C. in the microwave. The mixture was filtered and residue was purified by RP-HPLC (column: X-Bridge C18 5 μm 100×30 mm, mobile phase: acetonitrile/water (0.1 vol. % formic acid)-gradient) to give 20 mg of the title compound (95% purity, 19% yield).

¹H NMR (DMSO-d₆) δ: 8.02-8.14 (m, 1H), 7.94-8.02 (m, 1H), 7.22-7.39 (m, 3H), 7.08-7.18 (m, 2H), 4.75-4.85 (m, 1H), 3.70-3.82 (m, 5H), 3.49-3.62 (m, 2H), 2.14-2.27 (m, 2H), 1.87-1.99 (m, 2H), 1.81 (d, 6H).

LC-MS (Method 1): Rₜ=1.21 min; MS (ESIpos): m/z=520 [M+H]⁺

### Example 136

### 8-(methanesulfonyl)-1-methyl-2-oxo-4-{4-[4-(trifluoromethoxy)phenoxy]piperidin-1-yl}-1,2-dihydroquinoline-3-carbonitrile

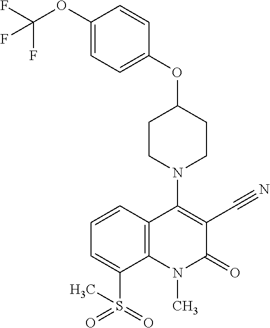 [SMILES: Cn1c(=O)c(C#N)c(N2CCC(Oc3ccc(OC(F)(F)F)cc3)CC2)c2cccc(S(C)(=O)=O)c21]

A solution of 940 mg 8-bromo-1-methyl-2-oxo-4-{4-[4-(trifluoromethoxy)phenoxy]piperidin-1-yl}-1,2-dihydroquinoline-3-carbonitrile (1.71 mmol, example 130) in 8 mL DMSO was degassed with argon. To the mixture was added 828 mg sodium methanesulfinate (7.68 mmol, CAS 20277-69-4), 252 μL (1S,2S)-cyclohexane-1,2-diamine (2.1 mmol, CAS 1121-22-8) and 258 mg copper(I)trifluoromethanesulfonate-benzene (2:2:1) (513 μmol). The reaction was stirred for 73 h at 110° C. The mixture was filtered and residue was purified by RP-HPLC (column: X-Bridge C18 5μ 100×30 mm; mobile phase: water (0.2 vol. % aq. ammonia 32%)/acetonitrile 45-85%) to give 100 mg of the title compound (98% purity, 11% yield).

¹H NMR (DMSO-d₆) δ: 8.35-8.46 (m, 1H), 8.04-8.16 (m, 1H), 7.37-7.47 (m, 1H), 7.27-7.36 (m, 2H), 7.11-7.20 (m, 2H), 4.74 (s, 1H), 3.69-3.82 (m, 2H), 3.51-3.65 (m, 8H), 2.16-2.27 (m, 2H), 1.86-2.02 (m, 2H).

LC-MS (Method 2): Rₜ=1.29 min; MS (ESIpos): m/z=522.5 [M+H]⁺

### Example 137

### 6-bromo-4-[4-(4-fluorophenoxy)piperidin-1-yl]-1-methyl-2-oxo-1,2-dihydroquinoline-3-carbonitrile

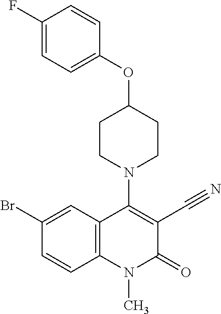 [SMILES: Cn1c(=O)c(C#N)c(N2CCC(Oc3ccc(F)cc3)CC2)c2cc(Br)ccc21]

A suspension of 300 mg 6-bromo-4-chloro-1-methyl-2-oxo-1,2-dihydroquinoline-3-carbonitrile (1.01 mmol, intermediate 62), 236 mg 4-(4-fluorophenoxy)piperidine (1.21 mmol, CAS 3202-34-4) and 530 μL N,N-diisopropylethylamine (3.0 mmol) in 10 mL 2-propanol was stirred for 2 h at 90° C. After this time, water was added and the reaction was stirred for some time. The residue was collected by filtration and washed with ethanol. The resulting solid was dried in vacuum at 100° C. to give 310 mg of the title compound (64% yield, 95% purity).

¹H NMR (DMSO-d₆) δ: 7.83-7.95 (m, 2H), 7.50-7.59 (m, 1H), 6.98-7.19 (m, 4H), 4.60-4.79 (m, 1H), 3.69-3.82 (m, 2H), 3.47-3.61 (m, 5H), 2.11-2.24 (m, 2H), 1.79-1.94 (m, 2H).

LC-MS (Method 1): Rₜ=1.41 min; MS (ESIpos): m/z=458 [M+H]⁺

### Example 138

### 6-bromo-4-[4-(3-methoxyphenoxy)piperidin-1-yl]-1-methyl-2-oxo-1,2-dihydroquinoline-3-carbonitrile

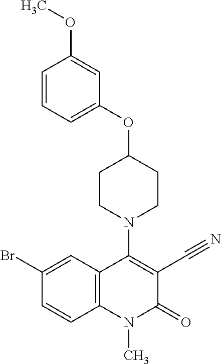 [SMILES: COc1cccc(OC2CCN(c3c(C#N)c(=O)n(C)c4ccc(Br)cc34)CC2)c1]

A suspension of 359 mg 6-bromo-4-chloro-1-methyl-2-oxo-1,2-dihydroquinoline-3-carbonitrile (1.21 mmol, intermediate 62), 250 mg 4-(3-methoxyphenoxy)piperidine (1.21 mmol, CAS 162402-37-1) and 340 μL N,N-diisopropylethylamine (2.4 mmol) in 8 mL 2-propanol was stirred for 3 h at 90° C. After this time, ethyl acetate was added and the reaction was refluxed for 15 min. The mixture was stirred for 48 h at rt. Water was added and the residue was collected by filtration. The impure product was purified by RP-HPLC (column: X-Bridge C18 5 μm 100×30 mm, mobile phase: acetonitrile/water (0.2 vol. % ammonia 32%)-gradient). 220 mg of the title compound were obtained (38% yield, 95% purity).

¹H NMR (DMSO-d₆) δ: 7.85-7.95 (m, 2H), 7.49-7.58 (m, 1H), 7.15-7.24 (m, 1H), 6.58-6.65 (m, 2H), 6.48-6.55 (m, 1H), 4.69-4.82 (m, 1H), 3.68-3.83 (m, 5H), 3.51-3.62 (m, 5H), 2.20 (ddd, 2H), 1.82-1.97 (m, 2H).

LC-MS (Method 2): Rₜ=1.34 min; MS (ESIpos): m/z=469.3 [M+H]⁺

### Example 139

### 6-bromo-1-methyl-2-oxo-4-{4-[4-(trifluoromethoxy)phenoxy]piperidin-1-yl}-1,2-dihydroquinoline-3-carbonitrile

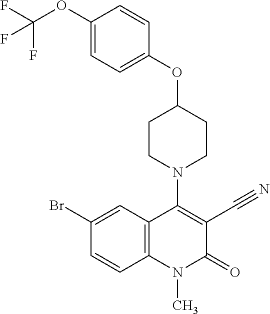 [SMILES: Cn1c(=O)c(C#N)c(N2CCC(Oc3ccc(OC(F)(F)F)cc3)CC2)c2cc(Br)ccc21]

A suspension of 260 mg 6-bromo-4-chloro-1-methyl-2-oxo-1,2-dihydroquinoline-3-carbonitrile (874 μmol, intermediate 62), 274 mg 4-[4-(trifluoromethoxy)phenoxy]piperidine (1.05 mmol, CAS 287952-67-4) and 460 μL N,N-diisopropylethylamine (2.6 mmol) in 10 mL 2-propanol was stirred for 2 h at 90° C. After this time, water was added and the reaction was stirred for some time. The residue was collected by filtration and washed with ethanol. The resulting solid was dried to give 320 mg of the title compound (67% yield, 95% purity).

¹H NMR (DMSO-d₆) δ: 7.82-7.96 (m, 2H), 7.51-7.60 (m, 1H), 7.27-7.36 (m, 2H), 7.10-7.20 (m, 2H), 4.74-4.87 (m, 1H), 3.70-3.84 (m, 2H), 3.50-3.65 (m, 5H), 2.21 (ddd, 2H), 1.82-1.98 (m, 2H).

LC-MS (Method 1): Rₜ=1.53 min; MS (ESIpos): m/z=524 [M+H]⁺

### Example 140

### 6-bromo-1-methyl-2-oxo-4-[4-(phenylsulfanyl)piperidin-1-yl]-1,2-dihydroquinoline-3-carbonitrile

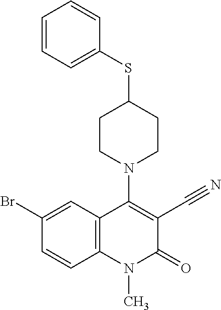 [SMILES: Cn1c(=O)c(C#N)c(N2CCC(Sc3ccccc3)CC2)c2cc(Br)ccc21]

A suspension of 300 mg 6-bromo-4-chloro-1-methyl-2-oxo-1,2-dihydroquinoline-3-carbonitrile (1.01 mmol, intermediate 62), 278 mg 4-(phenylsulfanyl)piperidine hydrogen chloride salt (1.21 mmol, CAS 101798-66-7) and 530 μL N,N-diisopropylethylamine (3.0 mmol) in 10 mL 2-propanol was stirred for 2 h at 90° C. After this ti me, water was added and the reaction was stirred for some time. The residue was collected by filtration and washed with ethanol. The resulting solid was dried to give 330 mg of the title compound (68% yield, 95% purity).

¹H NMR (DMSO-d₆) δ: 7.82-7.92 (m, 2H), 7.44-7.60 (m, 3H), 7.33-7.41 (m, 2H), 7.24-7.32 (m, 1H), 3.60-3.83 (m, 3H), 3.45-3.59 (m, 5H), 2.04-2.22 (m, 2H), 1.65-1.82 (m, 2H).

LC-MS (Method 1): Rₜ=1.47 min; MS (ESIpos): m/z=456 [M+H]⁺

### Example 141

### 4-[4-(4-fluorophenoxy)piperidin-1-yl]-1,6-dimethyl-2-oxo-1,2-dihydroquinoline-3-carbonitrile

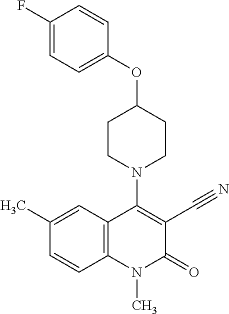 [SMILES: Cc1ccc2c(c1)c(N1CCC(Oc3ccc(F)cc3)CC1)c(C#N)c(=O)n2C]

A suspension of 142 mg 6-bromo-4-[4-(4-fluorophenoxy)piperidin-1-yl]-1-methyl-2-oxo-1,2-dihydroquinoline-3-carbonitrile (311 μmol, example 137), 177 mg 2,4,4,5,5-pentamethyl-1,3,2-dioxaborolane (1.24 mmol, CAS 94242-85-0), 1.1 mL tripotassium phosphate (0.50 M, 560 μmol) and 73.4 mg chloro(2-dicyclohexylphosphino-2′,4′,6′-triisopropyl-1,1′-biphenyl)[2-(2′-amino-1,1′-biphenyl)]palladium(II) (93 μmol, CAS 1310584-14-5) in 4.9 mL THF was stirred overnight at 70° C. Water was added and the reaction was extracted with dichloromethane (2×). The organic phase was washed with brine and dried over sodium sulfate. After evaporation, the residue was purified by flash chromatography (silica, hexane/ethyl acetate gradient 0-100%). The impure product was stirred in DMSO for some time. The precipitate was collected by filtration and dried in vacuum. 32 mg of the title compound were obtained (22% yield, 85% purity).

¹H NMR (DMSO-d₆) δ: 7.61-7.66 (m, 1H), 7.55-7.59 (m, 1H), 7.44-7.51 (m, 1H), 6.99-7.22 (m, 4H), 4.61-4.79 (m, 1H), 3.71-3.80 (m, 2H), 3.50-3.62 (m, 5H), 2.37-2.43 (m, 3H), 2.11-2.28 (m, 2H), 1.83-1.98 (m, 2H).

LC-MS (Method 1): Rₜ=1.32 min; MS (ESIpos): m/z=392 [M+H]⁺

### Example 142

### 1,6-dimethyl-2-oxo-4-[4-(phenylsulfanyl)piperidin-1-yl]-1,2-dihydroquinoline-3-carbonitrile

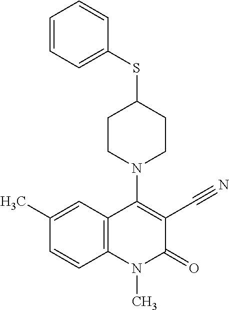 [SMILES: Cc1ccc2c(c1)c(N1CCC(Sc3ccccc3)CC1)c(C#N)c(=O)n2C]

A suspension of 138 mg 6-bromo-1-methyl-2-oxo-4-[4-(phenylsulfanyl)piperidin-1-yl]-1,2-dihydroquinoline-3-carbonitrile (304 μmol, example 140), 173 mg 2,4,4,5,5-pentamethyl-1,3,2-dioxaborolane (1.21 mmol, CAS 94242-85-0), 1.1 mL tripotassium phosphate (0.50 M, 550 μmol) and 72 mg chloro(2-dicyclohexylphosphino-2′,4′,6′-triisopropyl-1,1′-biphenyl)[2-(2′-amino-1,1′-biphenyl)]palladium(II) (91 μmol, CAS 1310584-14-5) in 4.8 mL THF was stirred overnight at 70° C. Water was added and the reaction was extracted with dichloromethane (2×). The organic phase was washed with brine and dried over sodium sulfate. After evaporation, the residue was purified by flash chromatography (silica, hexane/ethyl acetate gradient 0-100%). The impure product was purified by RP-HPLC (column: Chromatorex 125×30 mm, 10 μm mobile phase: acetonitrile/water (0.1 vol. % formic acid)-gradient). 7 mg of the title compound were obtained (6% yield, 95% purity).

¹H NMR (DMSO-d₆) δ: 7.52-7.61 (m, 2H), 7.43-7.51 (m, 3H), 7.33-7.42 (m, 2H), 7.22-7.32 (m, 1H), 3.60-3.84 (m, 3H), 3.41-3.57 (m, 5H), 2.37-2.43 (m, 3H), 2.06-2.21 (m, 2H), 1.71-1.91 (m, 2H).

LC-MS (Method 1): Rₜ=1.42 min; MS (ESIpos): m/z=390 [M+H]⁺

### Example 143

### 1,6-dimethyl-2-oxo-4-{4-[4-(trifluoromethoxy)phenoxy]piperidin-1-yl}-1,2-dihydroquinoline-3-carbonitrile

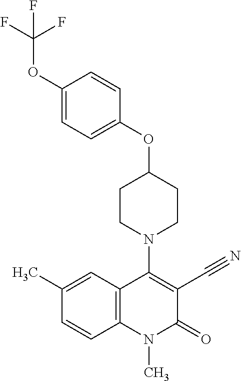 [SMILES: Cc1ccc2c(c1)c(N1CCC(Oc3ccc(OC(F)(F)F)cc3)CC1)c(C#N)c(=O)n2C]

A suspension of 142 mg 6-bromo-1-methyl-2-oxo-4-{4-[4-(trifluoromethoxy)phenoxy]piperidin-1-yl}-1,2-dihydroquinoline-3-carbonitrile (272 μmol, example 139), 77 mg 2,4,4,5,5-pentamethyl-1,3,2-dioxaborolane (544 μmol, CAS 94242-85-0), 1 mL tripotassium phosphate (0.50 M, 490 μmol) and 32 mg chloro(2-dicyclohexylphosphino-2′,4′,6′-triisopropyl-1,1′-biphenyl)[2-(2′-amino-1,1′-biphenyl)]palladium(II) (41 μmol, CAS 1310584-14-5) in 4.3 mL THF was stirred overnight at 70° C. Water was added and the reaction was extracted with dichloromethane (2×). The organic phase was washed with brine and dried over sodium sulfate. After evaporation, the residue was purified by flash chromatography (silica, hexane/ethyl acetate gradient 0-90%). The impure product was stirred in DMSO for some time. The precipitate was collected by filtration and dried in vacuum. 20 mg of the title compound were obtained (15% yield, 95% purity). The filtrate was purified by RP-HPLC (column: Chromatorex 125×30 mm, 10 μm mobile phase: acetonitrile/water (0.1 vol. % formic acid)-gradient). 34 mg of the title compound were obtained (26% yield, 95% purity).

¹H NMR (DMSO-d₆) δ: 7.62-7.66 (m, 1H), 7.54-7.60 (m, 1H), 7.45-7.52 (m, 1H), 7.26-7.37 (m, 2H), 7.09-7.19 (m, 2H), 4.68-4.89 (m, 1H), 3.69-3.88 (m, 2H), 3.48-3.63 (m, 5H), 2.39-2.44 (m, 3H), 2.16-2.28 (m, 2H), 1.84-2.01 (m, 2H).

LC-MS (Method 1): Rₜ=1.48 min; MS (ESIpos): m/z=458 [M+H]⁺

### Example 144

### 1-methyl-2-oxo-4-[4-(phenylsulfanyl)piperidin-1-yl]-1,2-dihydroquinoline-3,6-dicarbonitrile

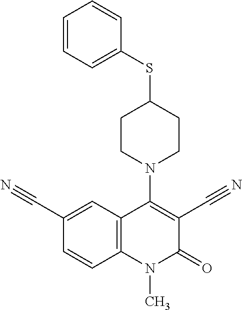 [SMILES: Cn1c(=O)c(C#N)c(N2CCC(Sc3ccccc3)CC2)c2cc(C#N)ccc21]

To a solution of 138 mg 6-bromo-1-methyl-2-oxo-4-[4-(phenylsulfanyl)piperidin-1-yl]-1,2-dihydroquinoline-3-carbonitrile (304 μmol, example 140) in 3.4 mL DMF was added 136 mg copper(I)cyanide (1.52 mmol, CAS 544-92-3) and the mixture was stirred for 12 h at 150° C. in the microwave. The mixture was cooled down to rt, a solution of 985 mg trichloroiron hexahydrate in 950 μL water and 160 μL concentrated hydrochloric acid was added and the mixture was stirred for 15 min. at 70° C. After cooled down to rt, water was added and the precipitate was collected by filtration and washed with ethanol. The resulting solid was purified by RP-HPLC (column: Chromatorex 125×30 mm, 10 μm mobile phase: acetonitrile/water (0.1 vol. % formic acid)-gradient). 13 mg of the title compound were obtained (10% yield, 95% purity).

¹H NMR (DMSO-d₆) δ: 8.15-8.22 (m, 1H), 8.06-8.13 (m, 1H), 7.65-7.76 (m, 1H), 7.43-7.50 (m, 2H), 7.33-7.41 (m, 2H), 7.24-7.31 (m, 1H), 3.73-3.85 (m, 2H), 3.60-3.73 (m, 1H), 3.48-3.59 (m, 5H), 2.07-2.17 (m, 2H), 1.76-1.91 (m, 2H).

LC-MS (Method 1): Rₜ=1.29 min; MS (ESIpos): m/z=401 [M+H]⁺

### Example 145

### 1-methyl-2-oxo-4-{4-[4-(trifluoromethoxy)phenoxy]piperidin-1-yl}-1,2-dihydroquinoline-3,6-dicarbonitrile

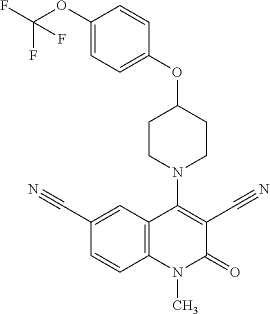 [SMILES: Cn1c(=O)c(C#N)c(N2CCC(Oc3ccc(OC(F)(F)F)cc3)CC2)c2cc(C#N)ccc21]

To a solution of 126 mg 6-bromo-1-methyl-2-oxo-4-{4-[4-(trifluoromethoxy)phenoxy]piperidin-1-yl}-1,2-dihydroquinoline-3-carbonitrile (241 μmol, example 139) in 2.7 mL DMF was added 108 mg copper(I)cyanide (1.21 mmol, CAS 544-92-3) and the mixture was stirred for 12 h at 150° C. in the microwave. The mixture was cooled down to rt, a solution of 782 mg trichloroiron hexahydrate in 760 μL water and 130 μL concentrated hydrochloric acid was added and the mixture was stirred for 15 min. at 70° C. After cooled down to rt, water was added and the precipitate was collected by filtration and washed with ethanol. The resulting solid was purified by RP-HPLC (column: Chromatorex 125×30 mm, 10 μm mobile phase: acetonitrile/water (0.1 vol. % formic acid)-gradient). 6 mg of the title compound were obtained (5% yield, 95% purity).

¹H NMR (DMSO-d₆) δ: 8.19-8.30 (m, 1H), 8.07-8.15 (m, 1H), 7.67-7.78 (m, 1H), 7.27-7.36 (m, 2H), 7.13-7.19 (m, 2H), 4.70-4.97 (m, 1H), 3.74-3.87 (m, 2H), 3.68 (br d, 5H), 2.16-2.29 (m, 2H), 1.87-2.03 (m, 2H).

LC-MS (Method 1): Rₜ=1.37 min; MS (ESIpos): m/z=469 [M+H]⁺

### Example 146

### 4-[4-(4-fluorophenoxy)piperidin-1-yl]-1-methyl-2-oxo-1,2-dihydroquinoline-3,6-dicarbonitrile

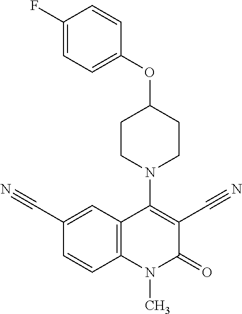 [SMILES: Cn1c(=O)c(C#N)c(N2CCC(Oc3ccc(F)cc3)CC2)c2cc(C#N)ccc21]

To a solution of 115 mg 6-bromo-4-[4-(4-fluorophenoxy)piperidin-1-yl]-1-methyl-2-oxo-1,2-dihydroquinoline-3-carbonitrile (252 μmol, example 137 in 2.9 mL DMF was added 113 mg copper(I)cyanide (1.26 mmol, CAS 544-92-3) and the mixture was stirred for 12 h at 150° C. in the microwave. The mixture was cooled down to rt, a solution of 817 mg trichloroiron hexahydrate in 790 μL water and 130 μL concentrated hydrochloric acid was added and the mixture was stirred for 15 min. at 70° C. After cooled down to rt, water was added and the precipitate was collected by filtration and washed with ethanol. The resulting solid was purified by RP-HPLC (column: Chromatorex 125×30 mm, 10 μm mobile phase: acetonitrile/water (0.1 vol. % formic acid)-gradient). 16 mg of the title compound were obtained (15% yield, 95% purity).

¹H NMR (DMSO-d₆) δ: 8.20-8.28 (m, 1H), 8.06-8.14 (m, 1H), 7.67-7.75 (m, 1H), 7.05-7.21 (m, 4H), 4.63-4.80 (m, 1H), 3.75-3.85 (m, 2H), 3.66 (s, 5H), 2.11-2.27 (m, 2H), 1.83-2.09 (m, 2H).

LC-MS (Method 1): Rₜ=1.24 min; MS (ESIpos): m/z=403 [M+H]⁺

### Example 147

### 6-cyclopropyl-1-methyl-2-oxo-4-[4-(phenylsulfanyl)piperidin-1-yl]-1,2-dihydroquinoline-3-carbonitrile

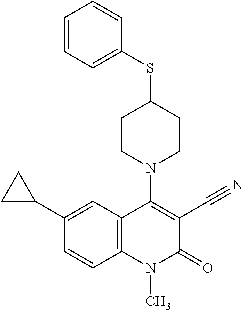 [SMILES: Cn1c(=O)c(C#N)c(N2CCC(Sc3ccccc3)CC2)c2cc(C3CC3)ccc21]

mg 6-bromo-1-methyl-2-oxo-4-[4-(phenylsulfanyl)piperidin-1-yl]-1,2-dihydroquinoline-3-carbonitrile (66.0 μmol, example 140 and 5.8 mg di-μ-iodobis(tri-tert-butylphosphino)dipalladium(I) (6.6 μmol, CAS 166445-62-1) were sealed in a vessel and degassed with argon. 1.0 mL toluene was added and the mixture was stirred at rt. 400 μL bromo(cyclopropyl)zinc (0.50 M, 200 μmol, CAS 126403-68-7) was added dropwise. The mixture was stirred at rt for 1 h. The mixture was filtered via a silica column. The column was washed with dichloromethane and dichloromethane/methanol (9:1). The filtrate was concentrated under reduced pressure. The residue was purified by RP-HPLC (column: X-Bridge C18 5 μm 100×30 mm, mobile phase: acetonitrile/water (0.1 vol. % formic acid)-gradient) to give 24.4 mg of the title compound (100% purity, 89% yield).

¹H NMR (ACETONITRILE-d₃) δ: 7.30-7.35 (m, 2H), 7.27-7.30 (m, 1H), 7.17-7.26 (m, 4H), 7.09-7.15 (m, 1H), 3.55-3.64 (m, 2H), 3.38-3.41 (m, 3H), 3.26-3.38 (m, 3H), 1.98-2.05 (m, 2H), 1.80-1.89 (m, 1H), 1.62-1.73 (m, 2H), 0.81-0.88 (m, 2H), 0.48-0.54 (m, 2H).

LC-MS (Method 1): Rₜ=1.47 min; MS (ESIpos): m/z=416.4 [M+H]⁺

### Example 148

### 6-cyclopropyl-4-[4-(4-fluorophenoxy)piperidin-1-yl]-1-methyl-2-oxo-1,2-dihydroquinoline-3-carbonitrile

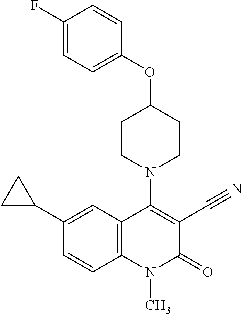 [SMILES: Cn1c(=O)c(C#N)c(N2CCC(Oc3ccc(F)cc3)CC2)c2cc(C3CC3)ccc21]

30 mg 6-bromo-4-[4-(4-fluorophenoxy)piperidin-1-yl]-1-methyl-2-oxo-1,2-dihydroquinoline-3-carbonitrile (65.7 μmol, example 137 and 5.7 mg di-μ-iodobis(tri-t-butylphosphino)dipalladium(I) (6.6 μmol, CAS 166445-62-1) were sealed in a vessel and degassed with argon. 1.0 mL toluene was added and the mixture was stirred at rt. 390 μL bromo(cyclopropyl)zinc (0.50 M, 200 μmol, CAS 126403-68-7) was added dropwise. The mixture was stirred at rt for 1 h. The mixture was filtered via a silica column. The column was washed with dichloromethane and dichloromethane/methanol (9:1). The filtrate was concentrated under reduced pressure. The residue was purified by RP-HPLC (column: X-Bridge C18 5 μm 100×30 mm, mobile phase: acetonitrile/water (0.1 vol. % formic acid)-gradient) to give 13.1 mg of the title compound (99% purity, 47% yield).

¹H NMR (ACETONITRILE-d₃) δ: 7.28-7.35 (m, 1H), 7.10-7.26 (m, 2H), 6.75-6.90 (m, 4H), 4.36-4.48 (m, 1H), 3.51-3.64 (m, 2H), 3.28-3.40 (m, 5H), 1.97-2.06 (m, 2H), 1.74-1.84 (m, 3H), 0.73-0.87 (m, 2H), 0.44-0.53 (m, 2H).

LC-MS (Method 1): Rₜ=1.42 min; MS (ESIpos): m/z=418.4 [M+H]⁺

### Example 149

### 1,6-dimethyl-4-[4-(3-methylphenoxy)piperidin-1-yl]-2-oxo-1,2-dihydroquinoline-3-carbonitrile

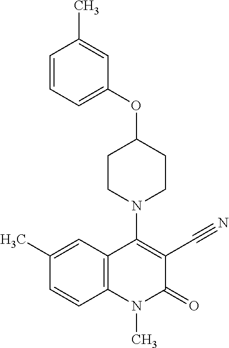 [SMILES: Cc1cccc(OC2CCN(c3c(C#N)c(=O)n(C)c4ccc(C)cc34)CC2)c1]

A solution of 150 mg 4-chloro-1,6-dimethyl-2-oxo-1,2-dihydroquinoline-3-carbonitrile (612 μmol, synthesis described in WO2004074218), 148 mg 4-(3-methylphenoxy)piperidine (735 μmol, CAS 63843-46-9) and 0.17 mL triethylamine (1.2 mmol) in 6.2 mL 2-propanol was stirred for 4 h at 90° C. After this time, water was added and the reaction was extracted with ethyl acetate. The organic phase was washed with water and brine and dried over sodium sulfate. After evaporation of the solvent, the residue was purified by RP-HPLC (column: X-Bridge C18 5 μm 100×30 mm, mobile phase: acetonitrile/water (0.2 vol. % ammonia 32%)-gradient). 110 mg of the title compound were obtained (95% purity, 44% yield).

¹H NMR (DMSO-d₆) δ: 7.52-7.66 (m, 2H), 7.42-7.49 (m, 1H), 7.11-7.22 (m, 1H), 6.79-6.90 (m, 2H), 6.71-6.77 (m, 1H), 4.67-4.81 (m, 1H), 3.70-3.82 (m, 2H), 3.50-3.64 (m, 5H), 2.39-2.42 (m, 3H), 2.27-2.30 (m, 3H), 2.15-2.24 (m, 2H), 1.86-1.96 (m, 2H).

LC-MS (Method 2): Rₜ=1.40 min; MS (ESIpos): m/z=388.5 [M+H]⁺

### Example 150

### 1,6-dimethyl-4-[4-(2-methylphenoxy)piperidin-1-yl]-2-oxo-1,2-dihydroquinoline-3-carbonitrile

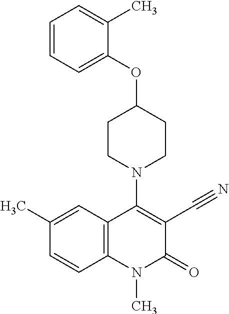 [SMILES: Cc1ccc2c(c1)c(N1CCC(Oc3ccccc3C)CC1)c(C#N)c(=O)n2C]

A solution of 150 mg 4-chloro-1,6-dimethyl-2-oxo-1,2-dihydroquinoline-3-carbonitrile (612 μmol, synthesis described in WO2004074218), 148 mg 4-(2-methylphenoxy)piperidine (735 μmol, CAS 63843-42-5) and 0.17 mL triethylamine (1.2 mmol) in 6.2 mL 2-propanol was stirred for 4.5 h at 90° C. After this time, water was added and the reaction was extracted with ethyl acetate. The organic phase was washed with water and brine and dried over sodium sulfate. After evaporation of the solvent, the residue was purified by RP-HPLC (column: X-Bridge C18 5 μm 100×30 mm, mobile phase: acetonitrile/water (0.2 vol. % ammonia 32%)-gradient). 40 mg of the title compound were obtained (95% purity, 16% yield).

¹H NMR (DMSO-d₆) δ: 7.61-7.66 (m, 1H), 7.53-7.60 (m, 1H), 7.40-7.49 (m, 1H), 7.11-7.22 (m, 2H), 7.04-7.09 (m, 1H), 6.80-6.89 (m, 1H), 4.69-4.86 (m, 1H), 3.70-3.81 (m, 2H), 3.48-3.61 (m, 5H), 2.37-2.44 (m, 3H), 2.14-2.26 (m, 5H), 1.86-1.99 (m, 2H).

LC-MS (Method 2): Rₜ=1.43 min; MS (ESIpos): m/z=388.5 [M+H]⁺

TABLE 8
synthesis in analogy to example 150.

| | | | Starting | |
|---|---|---|---|---|
| Example | Structure | IUPAC-Name | Materials | Analytics |
| 151 | 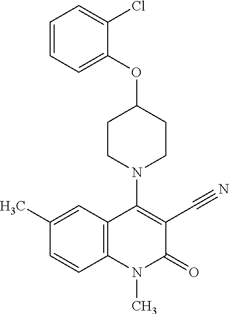 [SMILES: Cc1ccc2c(c1)c(N1CCC(Oc3ccccc3Cl)CC1)c(C#N)c(=O)n2C] | 4-[4-(2- chlorophenoxy) piperidin-1-yl]-1,6- dimethyl-2-oxo- 1,2- dihydroquinoline- 3-carbonitrile | 4-chloro-1,6- dimethyl-2-oxo- 1,2- dihydroquinoline- 3-carbonitrile (WO2004074218) and 4-(2- chlorophenoxy) piperidine (CAS 245057-65-2) | ¹H NMR (DMSO-d₆) δ: 7.54-7.67 (m, 2H), 7.40- 7.53 (m, 2H), 7.27-7.36 (m, 2H), 6.93-7.03 (m, 1H), 4.81-4.98 (m, 1H), 3.74-3.85 (m, 2H), 3.50- 3.66 (m, 5H), 2.37-2.44 (m, 3H), 2.16-2.28 (m, 2H), 1.91-2.03 (m, 2H). LC-MS (Method 2): Rₜ = 1.40 min; MS (ESIpos): m/z = 408.5 [M + H]⁺ |
| 152 | 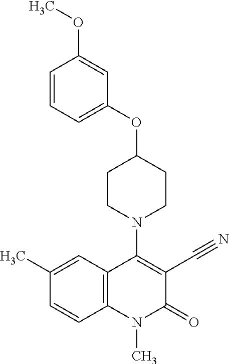 [SMILES: COc1cccc(OC2CCN(c3c(C#N)c(=O)n(C)c4ccc(C)cc34)CC2)c1] | 4-[4-(3- methoxyphenoxy) piperidin-1-yl]- 1,6-dimethyl-2- oxo-1 2- dihydroquinoline- 3-carbonitrile | 4-chloro-1,6- dimethyl-2-oxo- 1,2- dihydroquinoline- 3-carbonitrile (WO2004074218) and 4-(3- methoxyphenoxy) piperidine (CAS 162402- 37-1) | ¹H NMR (DMSO-d₆) δ: 7.52-7.70 (m, 2H), 7.44- 7.50 (m, 1H), 7.14-7.24 (m, 1H), 6.48-6.67 (m, 3H), 4.67-4.85 (m, 1H), 3.67-3.86 (m, 5H), 3.46- 3.61 (m, 5H), 2.37-2.45 (m, 3H), 2.13-2.28 (m, 2H), 1.84-1.98 (m, 2H). LC-MS (Method 2): Rₜ = 1.31 min; MS (ESIpos): m/z = 404.5 [M + H]⁺ |
| 153 | 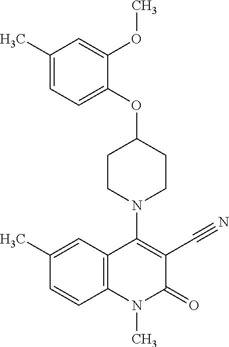 [SMILES: COc1cc(C)ccc1OC1CCN(c2c(C#N)c(=O)n(C)c3ccc(C)cc23)CC1] | 4-[4-(2-methoxy- 4- methylphenoxy) piperidin-1-yl]-1,6- dimethyl-2-oxo- 1,2- dihydroquinoline- 3-carbonitrile | 4-chloro-1,6- dimethyl-2-oxo- 1,2- dihydroquinoline- 3-carbonitrile (WO2004074218) and 4-(2- methoxy-4- methylphenoxy) piperidine (CAS 883543-21-3) | ¹H NMR (DMSO-d₆) δ: 7.53-7.67 (m, 2H), 7.43- 7.51 (m, 1H), 6.95-7.05 (m, 1H), 6.77-6.87 (m, 1H), 6.64-6.70 (m, 1H), 4.49-4.60 (m, 1H), 3.71- 3.83 (m, 5H), 3.44-3.59 (m, 5H), 2.37-2.45 (m, 3H), 2.23-2.29 (m, 3H), 2.08-2.22 (m, 2H), 1.81- 1.97 (m, 2H). LC-MS (Method 2): Rₜ = 1.36 min; MS (ESIpos): m/z = 418.5 [M + H]⁺ |
| 154 | 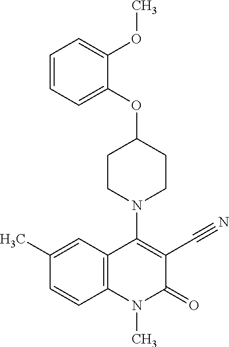 [SMILES: COc1ccccc1OC1CCN(c2c(C#N)c(=O)n(C)c3ccc(C)cc23)CC1] | 4-[4-(2- methoxyphenoxy) piperidin-1-yl]- 1,6-dimethyl-2- oxo-1,2- dihydroquinoline- 3-carbonitrile | 4-chloro-1,6- dimethyl-2-oxo- 1,2- dihydroquinoline- 3-carbonitrile (WO2004074218) and 4-(2- methoxyphenoxy) piperidine (CAS 28033-32- 1) | ¹H NMR (DMSO-d₆) δ: 7.52-7.68 (m, 2H), 7.44- 7.49 (m, 1H), 7.07-7.16 (m, 1H), 6.85-7.04 (m, 3H), 4.58-4.69 (m, 1H), 3.72-3.84 (m, 5H), 3.49- 3.59 (m, 5H), 2.41 (s, 3H), 2.17 (ddd, 2H), 1.85-2.01 (m, 2H). LC-MS (Method 2): Rₜ = 1.29 min; MS (ESIpos): m/z = 404.5 [M + H]⁺ |
| 155 | 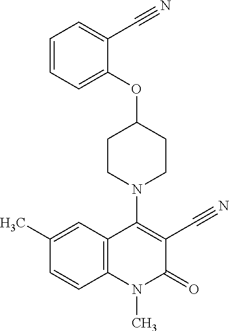 [SMILES: Cc1ccc2c(c1)c(N1CCC(Oc3ccccc3C#N)CC1)c(C#N)c(=O)n2C] | 4-[4-(2- cyanophenoxy) piperidin-1-yl]-1,6- dimethyl-2-oxo- 1,2- dihydroquinoline- 3-carbonitrile | 4-chloro-1,6- dimethyl-2-oxo- 1,2- dihydroquinoline- 3-carbonitrile (WO2004074218) and 2- (piperidin-4- yloxy)benzonitrile (CAS 900572- 37-4) | ¹H NMR (DMSO-d₆) δ: 7.76 (dd, J = 7.6, 1.5 Hz, 1H), 7.61-7.72 (m, 2H), 7.54-7.60 (m, 1H), 7.41- 7.52 (m, 2H), 7.07-7.16 (m, 1H), 4.92-5.04 (m, 1H), 3.71-3.85 (m, 2H), 3.54-3.65 (m, 5H), 2.41 (s, 3H), 2.21-2.31 (m, 2H), 1.92-2.05 (m, 2H). LC-MS (Method 2): Rₜ = 1.23 min; MS (ESIpos): m/z = 399.5 [M + H]⁺ |

### Example 156

### 6-methoxy-1-methyl-2-oxo-4-{4-[4-(trifluoromethoxy)phenoxy]piperidin-1-yl}-1,2-dihydroquinoline-3-carbonitrile

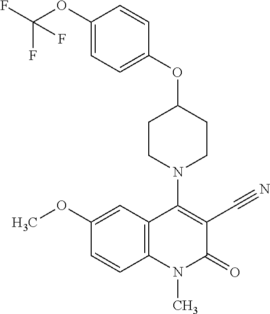 [SMILES: COc1ccc2c(c1)c(N1CCC(Oc3ccc(OC(F)(F)F)cc3)CC1)c(C#N)c(=O)n2C]

A solution of 100 mg 4-chloro-6-methoxy-1-methyl-2-oxo-1,2-dihydroquinoline-3-carbonitrile (402 μmol, intermediate 48), 126 mg 4-[4-(trifluoromethoxy)phenoxy]piperidine (483 μmol, CAS 287952-67-4) and 0.11 mL triethylamine (0.8 mmol) in 5.7 mL 2-propanol was stirred for 4.5 h at 90° C. After this time, water was added and the reaction was extracted with ethyl acetate. The organic phase was washed with water and brine and dried over sodium sulfate. After evaporation of the solvent, the residue was purified by RP-HPLC (column: X-Bridge C18 5 μm 100×30 mm, mobile phase: acetonitrile/water (0.2 vol. % ammonia 32%)-gradient). 35 mg of the title compound were obtained (95% purity, 17% yield).

¹H NMR (DMSO-d₆) δ: 7.48-7.56 (m, 1H), 7.36-7.43 (m, 1H), 7.27-7.34 (m, 2H), 7.20-7.25 (m, 1H), 7.12-7.19 (m, 2H), 4.72-4.84 (m, 1H), 3.87 (s, 3H), 3.70-3.82 (m, 2H), 3.50-3.62 (m, 5H), 2.23 (ddd, 2H), 1.83-2.01 (m, 2H).

LC-MS (Method 2): Rₜ=1.41 min; MS (ESIpos): m/z=474.5 [M+H]⁺

### Example 157

### 6-chloro-1-methyl-2-oxo-4-{4-[4-(trifluoromethoxy)phenoxy]piperidin-1-yl}-1,2-dihydroquinoline-3-carbonitrile

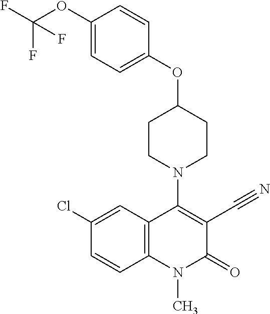 [SMILES: Cn1c(=O)c(C#N)c(N2CCC(Oc3ccc(OC(F)(F)F)cc3)CC2)c2cc(Cl)ccc21]

A solution of 90 mg 4,6-dichloro-1-methyl-2-oxo-1,2-dihydroquinoline-3-carbonitrile (356 μmol, intermediate 70), 111 mg 4-[4-(trifluoromethoxy)phenoxy]piperidine (427 μmol, CAS 287952-67-4) and 0.1 mL triethylamine (710 μmol) in 5 mL 2-propanol was stirred for 5 h at 90° C. After this time, water was added and the reaction was extracted with ethyl acetate. The organic phase was washed with water and brine and dried over sodium sulfate. After evaporation of the solvent, the residue was purified by RP-HPLC (column: X-Bridge C18 5 μm 100×30 mm, mobile phase: acetonitrile/water (0.2 vol. % ammonia 32%)-gradient). 82 mg of the title compound were obtained (95% purity, 46% yield).

¹H NMR (DMSO-d₆) δ: 7.84 (d, J=9.1 Hz, 1H), 7.51-7.59 (m, 1H), 7.26-7.37 (m, 3H), 7.10-7.22 (m, 2H), 4.73-4.90 (m, 1H), 3.95 (s, 3H), 3.82-3.92 (m, 2H), 3.55-3.73 (m, 2H), 2.17-2.30 (m, 2H), 1.83-2.04 (m, 2H).

LC-MS (Method 2): Rₜ=1.59 min; MS (ESIpos): m/z=478.5 [M+H]⁺

### Example 158

### 1,6-dimethyl-4-[4-(3-methylphenoxy)piperidin-1-yl]-2-oxo-1,2-dihydroquinoline-3-carboxamide

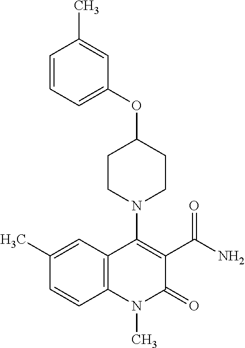 [SMILES: Cc1cccc(OC2CCN(c3c(C(N)=O)c(=O)n(C)c4ccc(C)cc34)CC2)c1]

A mixture of 71 mg 1,6-dimethyl-4-[4-(3-methylphenoxy)piperidin-1-yl]-2-oxo-1,2-dihydroquinoline-3-carbonitrile (174 μmol, example 149), 9.8 mg palladium(II)diacetate (44 μmol) and 103 mg N-[(1E)-ethylidene]hydroxylamine (1.74 mmol, CAS 107-29-9) in 10 mL ethanol was stirred for 4 h at 80° C. Water was added and the reaction was extracted with ethyl acetate (2×). The organic phase was washed with brine and dried over sodium sulfate. After evaporation the residue was purified by flash chromatography (silica, dichloromethane/methanol gradient 0-3%) to give 58 mg of the title compound (95% purity, 78% yield).

¹H NMR (DMSO-d₆) δ: 7.60-7.71 (m, 2H), 7.39-7.51 (m, 3H), 7.13-7.21 (m, 1H), 6.71-6.88 (m, 3H), 4.50-4.65 (m, 1H), 3.58 (s, 3H), 3.36 (br d, 2H), 3.04-3.19 (m, 2H), 2.41 (s, 3H), 2.27 (s, 3H), 2.08-2.17 (m, 2H), 1.78-1.93 (m, 2H).

LC-MS (Method 2): Rₜ=1.25 min; MS (ESIpos): m/z=406.6 [M+H]⁺

### Example 159

### 4-[4-(3-methoxyphenoxy)piperidin-1-yl]-1,6-dimethyl-2-oxo-1,2-dihydroquinoline-3-carboxamide

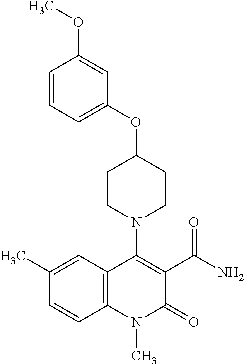 [SMILES: COc1cccc(OC2CCN(c3c(C(N)=O)c(=O)n(C)c4ccc(C)cc34)CC2)c1]

A mixture of 71 mg 4-[4-(3-methoxyphenoxy)piperidin-1-yl]-1,6-dimethyl-2-oxo-1,2-dihydroquinoline-3-carbonitrile (167 μmol, example 152), 9.4 mg palladium(II)diacetate (42 μmol) and 99 mg N-[(1E)-ethylidene]hydroxylamine (1.67 mmol, CAS 107-29-9) in 10 mL ethanol was stirred for 4 h at 80° C. Water was added and the reaction was extracted with ethyl acetate (2×). The organic phase was washed with brine and dried over sodium sulfate. After evaporation the residue was purified by flash chromatography (silica, dichloromethane/methanol gradient 0-3%) to give 60 mg of the title compound (95% purity, 81% yield).

¹H NMR (DMSO-d₆) δ: 7.61-7.72 (m, 2H), 7.38-7.49 (m, 3H), 7.13-7.23 (m, 1H), 6.46-6.64 (m, 3H), 4.49-4.68 (m, 1H), 3.73 (s, 3H), 3.58 (s, 3H), 3.34-3.41 (m, 2H), 3.09-3.20 (m, 2H), 2.41 (s, 3H), 2.09-2.17 (m, 2H), 1.79-1.92 (m, 2H).

LC-MS (Method 2): Rₜ=1.18 min; MS (ESIpos): m/z=422.6 [M+H]⁺

TABLE 9
synthesis in analogy to example 159.

| | | | Starting | |
|---|---|---|---|---|
| Example | Structure | IUPAC-Name | Materials | Analytics |
| 160 | 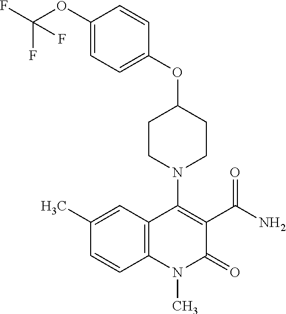 [SMILES: Cc1ccc2c(c1)c(N1CCC(Oc3ccc(OC(F)(F)F)cc3)CC1)c(C(N)=O)c(=O)n2C] | 1,6-dimethyl-2- oxo-4-{4-[4- (trifluoromethoxy) phenoxy] piperidin-1-yl}-1,2- dihydroquinoline-3- carboxamide | 1,6-dimethyl-2- oxo-4-{4-[4- (trifluoromethoxy) phenoxy]piperidin- 1-yl}-1,2- dihydroquinoline- 3-carbonitrile (example 143) | ¹H NMR (DMSO-d₆) δ: 7.67 (s, 2H), 7.39-7.50 (m, 3H), 7.27-7.33 (m, 2H), 7.07-7.19 (m, 2H), 4.56-4.69 (m, 1H), 3.57 (s, 3H), 3.38 (br s, 2H), 3.08-3.22 (m, 2H), 2.41 (s, 3H), 2.07-2.21 (m, 2H), 1.74-1.99 (m, 2H). LC-MS (Method 2): Rₜ = 1.34 min; MS (ESIpos): m/z = 476.5 [M + H]⁺ |
| 161 | 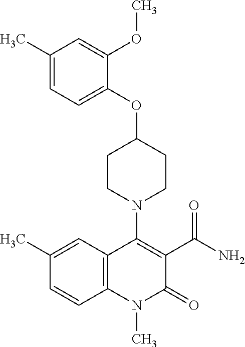 [SMILES: COc1cc(C)ccc1OC1CCN(c2c(C(N)=O)c(=O)n(C)c3ccc(C)cc23)CC1] | 4-[4-(2- methoxy-4- methylphenoxy) piperidin-1-yl]- 1,6-dimethyl-2- oxo-1,2- dihydroquinoline-3- carboxamide | 4-[4-(2-methoxy- 4- methylphenoxy) piperidin-1-yl]-1,6- dimethyl-2-oxo- 1,2- dihydroquinoline- 3-carbonitrile (example 153) | ¹H NMR (DMSO-d₆) δ: 7.60-7.73 (m, 2H), 7.37-7.52 (m, 3H), 6.88-7.00 (m, 1H), 6.78-6.86 (m, 1H), 6.66 (dt, 1H), 4.31-4.42 (m, 1H), 3.76 (s, 3H), 3.57 (s, 3H), 3.34-3.40 (m, 2H), 2.99-3.14 (m, 2H), 2.41 (s, 3H), 2.24 (s, 3H), 2.04-2.10 (m, 2H), 1.75-1.89 (m, 2H). LC-MS (Method 2): Rₜ = 1.20 min; MS (ESIpos): m/z = 436.6 [M + H]⁺ |
| 162 | 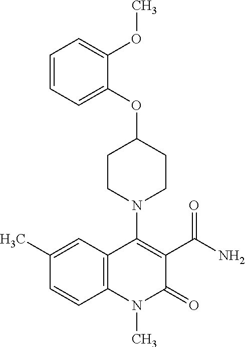 [SMILES: COc1ccccc1OC1CCN(c2c(C(N)=O)c(=O)n(C)c3ccc(C)cc23)CC1] | 4-[4-(2- methoxyphenoxy) piperidin-1- yl-1,6- dimethyl-2-oxo- 1,2- dihydroquinoline- 3-carboxamide | 4-[4-(2- methoxyphenoxy) piperidin-1-yl]- 1,6-dimethyl-2- oxo-1,2- dihydroquinoline- 3-carbonitrile (example 154) | ¹H NMR (DMSO-d₆) δ: 7.61-7.73 (m, 2H), 7.40-7.47 (m, 3H), 7.04-7.12 (m, 1H), 6.97-7.02 (m, 1H), 6.83-6.96 (m, 2H), 4.37-4.56 (m, 1H), 3.78 (s, 3H), 3.56 (s, 3H), 3.34-3.43 (m, 2H), 3.06-3.18 (m, 2H), 2.42 (s, 3H), 2.03-2.15 (m, 2H), 1.76-1.96 (m, 2H). LC-MS (Method 2): Rₜ = 1.12 min; MS (ESIpos): m/z = 422.5 [M + H]⁺ |
| 163 | 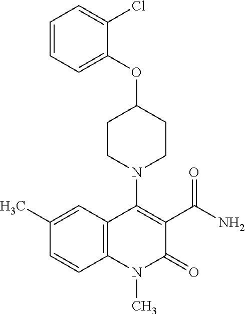 [SMILES: Cc1ccc2c(c1)c(N1CCC(Oc3ccccc3Cl)CC1)c(C(N)=O)c(=O)n2C] | 4-[4-(2- chlorophenoxy) piperidin-1-yl]- 1,6-dimethyl-2- oxo-1,2- dihydroquinoline- 3-carboxamide | 4-[4-(2- chlorophenoxy) piperidin-1-yl]-1,6- dimethyl-2-oxo- 1,2- dihydroquinoline- 3-carbonitrile (example 151) | ¹H NMR (DMSO-d₆) δ: 7.59-7.77 (m, 2H), 7.40-7.51 (m, 4H), 7.24-7.34 (m, 2H), 6.90-7.05 (m, 1H), 4.63-4.76 (m, 1H), 3.56 (s, 3H), 3.36-3.46 (m, 2H), 3.09-3.24 (m, 2H), 2.42 (s, 3H), 2.09-2.19 (m, 2H), 1.82-1.98 (m, 2H). LC-MS (Method 2): Rₜ = 1.24 min; MS (ESIpos): m/z = 426.4 [M + H]⁺ |
| 164 | 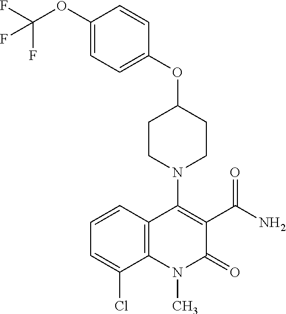 [SMILES: Cn1c(=O)c(C(N)=O)c(N2CCC(Oc3ccc(OC(F)(F)F)cc3)CC2)c2cccc(Cl)c21] | 8-chloro-1- methyl-2-oxo- 4-{4-[4- (trifluoromethoxy) phenoxy] piperidin-1-yl}-1,2- dihydroquinoline- 3-carboxamide | 8-chloro-1- methyl-2-oxo-4- (4-4- (trifluoromethoxy) phenoxy]piperidin- 1-yl}-1,2- dihydroquinoline- 3-carbonitrile (example 131) | ¹H NMR (DMSO-d₆) δ: 7.90 (dd, 1H), 7.65- 7.77 (m, 2H), 7.48-7.58 (m, 1H), 7.25-7.33 (m, 3H), 7.08-7.14 (m, 2H), 4.63 (dt, 1H), 3.73 (s, 3H), 3.08-3.19 (m, 2H), 2.07-2.17 (m, 2H), 1.79-1.91 (m, 2H). LC-MS (Method 2): Rₜ = 1.36 min; MS (ESIpos): m/z = 496.5 [M + H]⁺ |
| 165 | 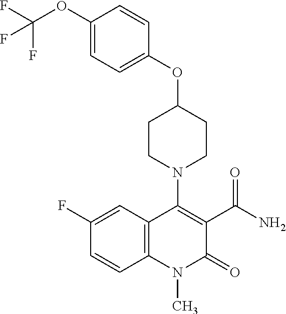 [SMILES: Cn1c(=O)c(C(N)=O)c(N2CCC(Oc3ccc(OC(F)(F)F)cc3)CC2)c2cc(F)ccc21] | 6-fluoro-1- methyl-2-oxo- 4-{4-[4- (trifluoromethoxy) phenoxy] piperidin-1-yl}-1,2- dihydroquinoline- 3-carboxamide | 6-fluoro-1- methyl-2-oxo-4- (4-4- (trifluoromethoxy) phenoxy]piperidin- 1-yl}-1,2- dihydroquinoline- 3-carbonitrile (example 166) | ¹H NMR (DMSO-d₆) δ: 7.47-7.60 (m, 3H), 7.28 (d, 2H), 7.06-7.12 (m, 2H), 4.56-4.65 (m, 1H), 3.57 (s, 3H),3.36 (br d, 2H), 3.06-3.17 (m, 2H), 2.12 (br dd, 2H), 1.78- 1.89 (m, 2H). LC-MS (Method 2): Rₜ = 1.29 min; MS (ESIpos): m/z = 480.5 [M + H]⁺ |

### Example 166

### 6-fluoro-1-methyl-2-oxo-4-{4-[4-(trifluoromethoxy)phenoxy]piperidin-1-yl}-1,2-dihydroquinoline-3-carbonitrile

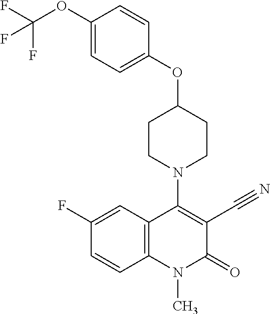 [SMILES: Cn1c(=O)c(C#N)c(N2CCC(Oc3ccc(OC(F)(F)F)cc3)CC2)c2cc(F)ccc21]

A suspension of 120 mg 4-chloro-6-fluoro-1-methyl-2-oxo-1,2-dihydroquinoline-3-carbonitrile (507 μmol, CAS 749865-80-3, synthesis described in WO2004074218), 164 mg 4-[4-(trifluoromethoxy)phenoxy]piperidine (535 μmol, CAS 287952-67-4) and 260 μL N,N-diisopropylethylamine (1.3 mmol) in 3 mL 2-propanol was stirred for 2 h at 90° C. After this time, water was added and the reaction was stirred for some time. The residue was collected by filtration and washed with water and ethanol. The resulting solid was dried to give 209 mg of the title compound (88% yield, 98% purity).

¹H NMR (DMSO-d₆) δ: 7.61-7.71 (m, 2H), 7.57 (dd, 1H), 7.31 (d, 2H), 7.12-7.18 (m, 2H), 4.77 (tt, 1H), 3.71-3.80 (m, 2H), 3.51-3.60 (m, 5H), 2.21 (ddd, 2H), 1.87-1.97 (m, 2H).

LC-MS (Method 2): Rₜ=1.43 min; MS (ESIpos): m/z=462.4 [M+H]⁺

### Example 167

### 6-hydroxy-1-methyl-2-oxo-4-{4-[4-(trifluoromethoxy)phenoxy]piperidin-1-yl}-1,2-dihydroquinoline-3-carbonitrile

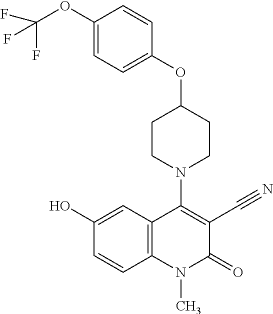 [SMILES: Cn1c(=O)c(C#N)c(N2CCC(Oc3ccc(OC(F)(F)F)cc3)CC2)c2cc(O)ccc21]

A solution of 100 mg 4-chloro-6-hydroxy-1-methyl-2-oxo-1,2-dihydroquinoline-3-carbonitrile (426 μmol, intermediate 63), 111 mg 4-[4-(trifluoromethoxy)phenoxy]piperidine (426 μmol, CAS 287952-67-4) and 0.12 mL triethylamine (850 μmol) in 5 mL 2-propanol was stirred for 2 h at 90° C. After this time, water and ethyl acetate were added and the reaction was stirred for some time. The solid that precipitated from this procedure was collected by filtration, the residue was purified by flash chromatography (silica, dichloromethane/methanol gradient 0-6%). 45 mg of the title compound were obtained (95% purity, 22% yield).

¹H NMR (DMSO-d₆) δ: 9.85 (br s, 1H), 7.40-7.50 (m, 1H), 7.28-7.37 (m, 2H), 7.10-7.26 (m, 4H), 4.72-4.91 (m, 1H), 3.65-3.79 (m, 2H), 3.42-3.59 (m, 5H), 2.14-2.29 (m, 2H), 1.84-1.99 (m, 2H).

LC-MS (Method 2): Rₜ=1.08 min; MS (ESIpos): m/z=460.4 [M+H]⁺

### Example 168

### 8-bromo-1,6-dimethyl-2-oxo-4-{4-[4-(trifluoromethoxy)phenoxy]piperidin-1-yl}-1,2-dihydroquinoline-3-carbonitrile

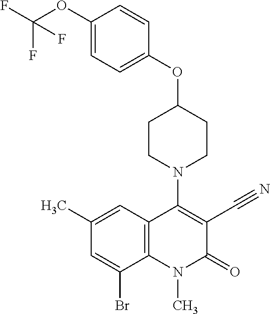 [SMILES: Cc1cc(Br)c2c(c1)c(N1CCC(Oc3ccc(OC(F)(F)F)cc3)CC1)c(C#N)c(=O)n2C]

A solution of 300 mg 8-bromo-4-chloro-1,6-dimethyl-2-oxo-1,2-dihydroquinoline-3-carbonitrile (963 μmol, intermediate 66), 291 mg 4-[4-(trifluoromethoxy)phenoxy]piperidine (1.06 mmol, CAS 287952-67-4) and 0.27 mL triethylamine (1.9 mmol) in 13 mL 2-propanol was stirred for 3 h at 90° C. After this time, water was added and the reaction was extracted with ethyl acetate. The organic phase was washed with water and brine and dried over sodium sulfate. After evaporation of the solvent, the residue was purified by flash chromatography (silica, dichloromethane/methanol gradient 0-2%). 520 mg of the title compound were obtained (95% purity, 96% yield).

¹H NMR (DMSO-d₆) δ: 7.81-7.95 (m, 1H), 7.56-7.68 (m, 1H), 7.24-7.36 (m, 2H), 7.08-7.19 (m, 2H), 4.69-4.83 (m, 1H), 3.71-3.82 (m, 2H), 3.68 (s, 3H), 3.50-3.61 (m, 2H), 2.38 (s, 3H), 2.14-2.25 (m, 2H), 1.83-2.02 (m, 2H).

LC-MS (Method 2): Rₜ=1.52 min; MS (ESIpos): m/z=538.4 [M+H]⁺

### Example 169

### 8-bromo-4-[4-(3-chlorophenoxy)piperidin-1-yl]-1,6-dimethyl-2-oxo-1,2-dihydroquinoline-3-carbonitrile

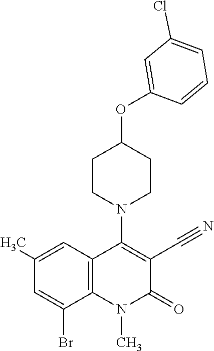 [SMILES: Cc1cc(Br)c2c(c1)c(N1CCC(Oc3cccc(Cl)c3)CC1)c(C#N)c(=O)n2C]

A solution of 300 mg 8-bromo-4-chloro-1,6-dimethyl-2-oxo-1,2-dihydroquinoline-3-carbonitrile (963 μmol, intermediate 66), 224 mg 4-(3-chlorophenoxy)piperidine (1.06 mmol, CAS 97840-40-9) and 0.27 mL triethylamine (1.9 mmol) in 13 mL 2-propanol was stirred for 3 h at 90° C. After this time, water was added and the reaction was extracted with ethyl acetate. The organic phase was washed with water and brine and dried over sodium sulfate. After evaporation of the solvent, the residue was purified by flash chromatography (silica, dichloromethane/methanol gradient 0-2%). 315 mg of the title compound were obtained (95% purity, 64% yield).

¹H NMR (DMSO-d₆) δ: 7.83-7.93 (m, 1H), 7.59-7.66 (m, 1H), 7.30-7.39 (m, 1H), 7.12-7.18 (m, 1H), 6.97-7.07 (m, 2H), 4.76-4.88 (m, 1H), 3.71-3.80 (m, 2H), 3.67 (s, 3H), 3.52-3.61 (m, 2H), 2.39 (s, 3H), 2.15-2.24 (m, 2H), 1.84-1.96 (m, 2H).

LC-MS (Method 2): Rₜ=1.52 min; MS (ESIpos): m/z=488.5 [M+H]⁺

### EXPERIMENTAL SECTION—BIOLOGICAL ASSAYS

### Human DGKα Kinase Activity Inhibition Assay

Human DGKα inhibitory activity of compounds of the present invention was quantified employing the human DGKα kinase activity assay as described in the following paragraphs. In essence, the enzyme activity was measured by quantification of the adenosine-di-phosphate (ADP) generated as a co-product of the enzyme reaction via the “ADP-Glo™ Kinase Assay” kit from the company Promega. This detection system works as follows: In a first step the ATP not consumed in the kinase reaction is quantitatively converted to cAMP employing an adenylate cyclase (“ADP-Glo-reagent”), then the adenylate cyclase is stopped and the ADP generated in the kinase reaction converted to ATP, which subsequently generates in a luciferase-based reaction a glow-luminescence signal (“Kinase Detection Reagent”).

C-terminally FLAG-tagged, recombinant full-length human DGKα (expressed in baculovirus infected insect cells, purified using anti-Flag pulldown and size exclusion chromatography as described below, DGKα_hu_1) was used as enzyme. As substrate for the kinase 1,2-dioleoyl-sn-glycerol, reconstituted in octyl-β-D-glucopyranoside micelles, was used. For the preparation of the micelles, 1 volume of a 16.1 mM solution of 1,2-dioleoyl-sn-glycerol (Avanti, Cat. #O8001-25G) in chloroform was slowly evaporated using a nitrogen stream. Subsequently, 22.55 volumes of a 510 mM solution of octyl-β-D-glucopyranoside (Sigma-Aldrich, Cat. #O8001-10G) in 50 mM MOPS buffer (pH 7.4) were added, and the mixture was sonicated in an ultrasonic bath for 20 s. Then 35 volumes of 50 mM MOPS buffer (pH 7.4) were added to yield a solution of 0.28 mM 1,2 dioleoyl-sn-glycerol and 200 mM octyl-β-D-glucopyranoside, which was aliquoted, flash-frozen in liquid nitrogen, and stored at −20° C. until use. For each experiment, a fresh aliquot was quickly thawed and diluted 24-fold with aqueous assay buffer (described below) containing 95.7 μM adenosine triphosphate (Promega) to yield a 1.67-fold concentrated substrate solution.

For the assay 50 nl of a 100-fold concentrated solution of the test compound in dimethyl sulfoxide (DMSO, Sigma) was pipetted into either a white 1536-well or a white low-volume 384-well microtiter plate (both Greiner Bio-One, Frickenhausen, Germany). Subsequently, 2 μl of a solution of human DGKα in aqueous assay buffer [50 mM (3-(N-morpholino)propanesulfonic acid (MOPS, pH 7.4, Sigma-Aldrich), 1 mM dithiothreitol (DTT, Sigma-Aldrich), 100 mM NaCl (Sigma-Aldrich), 10 mM MgCl₂ (Sigma-Aldrich), 0.1% (w/v) bovine gamma globulin (BGG, Sigma-Aldrich), 1 μM CaCl₂ (Sigma-Aldrich)] were added to the wells, and the mixture was incubated for 15 min at 22° C. to allow pre-binding of the test compounds to the enzyme. The reaction was initiated by the addition of 3 μl of substrate solution [preparation described above; 11.7 μM 1,2-dioleoyl-sn-glycerol (=>final conc. in the 5 μl assay volume is 7 μM), 8.33 mM octyl-β-D-glucopyranoside (=>final conc. in 5 μl assay volume is 5 mM), and 91.67 μM adenosine triphosphate (=>final conc. in 5 μl assay volume is 55 μM) in assay buffer] and the resulting mixture was incubated for a reaction time of 20 min at 22° C. The concentration of DGK a was adjusted depending of the activity of the enzyme lot and was chosen appropriate to have the assay in the linear range, a typical concentration is about 0.1 nM. The reaction was stopped by the addition of 2.5 μl of “ADP-Glo-reagent” (1 to 1.5 diluted with water) and the resulting mixture was incubated at 22° C. for 1 h to convert the ATP not consumed in the kinase reaction completely to cAMP. Subsequently 2.5 μl of the “kinase detection reagent” (1.2-fold more concentrated than recommended by the producer) were added, the resulting mixture was incubated at 22° C. for 1 h and then the luminescence measured with a suitable measurement instrument (e.g. Viewlux™ from Perkin-Elmer). The amount of emitted light was taken as a measure for the amount of ADP generated and thereby for the activity of the DGKα.

The data were normalised (enzyme reaction without inhibitor=0% inhibition, all other assay components but no enzyme=100% inhibition). Usually the test compounds were tested on the same microtiterplate in 11 different concentrations in the range of 20 μM to 0.07 nM (20 μM, 5.7 μM, 1.6 μM, 0.47 μM, 0.13 μM, 38 nM, 11 nM, 3.1 nM, 0.9 nM, 0.25 nM and 0.07 nM, the dilution series prepared separately before the assay on the level of the 100-fold concentrated solutions in DMSO by serial dilutions, exact concentrations may vary depending pipettors used) in duplicate values for each concentration and IC₅₀ values were calculated using Genedata Screener™ software.

TABLE 10
IC₅₀ values of examples in in vitro human
DGKα kinase activity inhibition assays.

| Example | IC₅₀ [nM] |
|---|---|
| 1 | 4.2 |
| 2 | 3.9 |
| 3 | 6.2 |
| 4 | 6.3 |
| 5 | 7.7 |
| 6 | 8.4 |
| 7 | 8.6 |
| 8 | 13 |
| 9 | 9.1 |
| 10 | 10 |
| 11 | 11 |
| 12 | 12 |
| 13 | 14 |
| 14 | 14 |
| 15 | 14 |
| 16 | 33 |
| 17 | 45 |
| 18 | 55 |
| 19 | 64 |
| 20 | 81 |
| 21 | 119 |
| 22 | 129 |
| 23 | 181 |
| 24 | 266 |
| 25 | 283 |
| 26 | 415 |
| 27 | 318 |
| 28 | 521 |
| 29 | 654 |
| 30 | 1530 |
| 31 | 6.8 |
| 32 | 31 |
| 33 | 13 |
| 34 | 9 |
| 35 | 646 |
| 36 | 164 |
| 37 | 10 |
| 38 | 18 |
| 39 | 4 |
| 40 | 11 |
| 41 | 173 |
| 42 | 27 |
| 43 | 339 |
| 44 | 45 |
| 45 | 31 |
| 46 | 32 |
| 47 | 8.6 |
| 48 | 0.9 |
| 49 | 1.7 |
| 50 | 3.1 |
| 51 | 6.1 |
| 52 | 3.8 |
| 53 | 1.1 |
| 54 | 5.8 |
| 55 | 4.4 |
| 56 | 9.9 |
| 57 | 17 |
| 58 | 19 |
| 59 | 18 |
| 60 | 27 |
| 61 | 22 |
| 62 | 2.3 |
| 63 | 12 |
| 64 | 16 |
| 65 | 7.3 |
| 66 | 14 |
| 67 | 6.4 |
| 68 | 29 |
| 69 | 507 |
| 70 | 17 |
| 71 | 5.7 |
| 72 | 16 |
| 73 | 13 |
| 74 | 3.5 |
| 75 | 3.7 |
| 76 | 6.8 |
| 77 | 17 |
| 78 | 1130 |
| 79 | 416 |
| 80 | 534 |
| 81 | 3 |
| 82 | 12 |
| 83 | 14 |
| 84 | 25 |
| 85 | 28 |
| 86 | 32 |
| 87 | 40 |
| 88 | 5.8 |
| 89 | 4.7 |
| 90 | 1.7 |
| 91 | 4.5 |
| 92 | 6.2 |
| 93 | 39 |
| 94 | 28 |
| 95 | 15 |
| 96 | 12 |
| 97 | 122 |
| 98 | 1.9 |
| 99 | 1.2 |
| 100 | 8.3 |
| 101 | 7.4 |
| 102 | 18 |
| 103 | 18 |
| 104 | 40 |
| 105 | 33 |
| 106 | 8.3 |
| 107 | 7.2 |
| 108 | 10 |
| 109 | 16 |
| 110 | 5.3 |
| 111 | 4.8 |
| 112 | 10 |
| 113 | 12 |
| 114 | 16 |
| 115 | 29 |
| 116 | 6.4 |
| 117 | 6. |
| 118 | 123 |
| 119 | 34 |
| 120 | 51 |
| 121 | 16 |
| 122 | 7.1 |
| 123 | 14 |
| 124 | 30 |
| 125 | 43 |
| 126 | 14 |
| 127 | 14 |
| 128 | 29 |
| 129 | 29 |
| 130 | 202 |
| 131 | 18 |
| 132 | 536 |
| 133 | 55 |
| 134 | 37 |
| 135 | 1370 |
| 136 | 2.5 |
| 137 | 1.9 |
| 138 | 1.5 |
| 139 | 2.5 |
| 140 | 1.4 |
| 141 | 7.7 |
| 142 | 2 |
| 143 | 4.7 |
| 144 | 3 |
| 145 | 4.4 |
| 146 | 17 |
| 147 | 32 |
| 148 | 73 |
| 149 | 7.7 |
| 150 | 50 |
| 151 | 60 |
| 152 | 3.8 |
| 153 | 10 |
| 154 | 13 |
| 155 | 9.3 |
| 156 | 7 |
| 157 | 1240 |
| 158 | 12 |
| 159 | 12 |
| 160 | 3.1 |
| 161 | 21 |
| 162 | 57 |
| 163 | 59 |
| 164 | 37 |
| 165 | 0.17 |
| 166 | 0.97 |
| 167 | 0.94 |
| 168 | 556 |
| 169 | 2530 |

### Transactivation Assay in Jurkat I1L2-Reporter Cell Line

Transactivation assays were carried out in Jurkat cells purchased from Promega (Promega, #CS187001) stably transfected with a firefly luciferase reporter gene construct under the control of the IL2-promoter. Cells were cultured as specified by the manufacturer. Bulk cells were harvested at a culture density of approx. 1E+06 cells/ml, suspended in cryo-storage medium (70% RPMI/20% FCS/10% DMSO), frozen at controlled rate of −1°/min in 1.8 ml cryo-vials with cell densities of 1E+07 to 1E+08 cells per vial, and stored at −150° C. or below until further use. Frozen cells were thawed and cultured in medium at a starting density of 3.5E+05 cells/ml for 6 days. On day 6 cells were centrifuged for 5 min at 300×g, medium was decanted and cell concentration was adjusted to 5.0E+06 cells/ml with fresh assay medium (500 ml RPMI (Gibco, #22400)+5 ml L-Glutamin (Sigma, #G7513)+5 ml Penicillin/Streptomycin (Sigma #P0781)+5 ml Non-essential amino acids (Invitrogen, #11140)+5 ml sodium-pyruvate (Gibco #1136088), 5 ml FBS (Biochrom, #S0615)). Cell working stock was split in two parts: neutral control and compounds with EC30 stimulation, high control with EC100 stimulation.

An antibody premix was prepared by diluting anti-CD3 (BD Pharmingen, #555329), anti-CD28 (BD Pharmingen, #555725) and goat anti mouse anti-IgG (ThermoFisher, #31160) antibodies at 1/1/4 ratio in assay medium at 2-fold of final concentration (final concentrations depend on cell batch, typically for neutral control 0.055/0.055/0.22 μg/ml, for high control 0.5/0.5/2 mg/ml). The premix solutions were added to the cells in 1+1 volume prior use.

Fifty nl of a 100-fold concentrated solution of the test compounds in DMSO were transferred into a white microtiter test plate (384, Greiner Bio-One, Germany). For this, either a Hummingbird liquid handler (Digilab, USA) or an Echo acoustic system (Labcyte, USA) was used. Five μl of the freshly prepared cell suspension was added to the wells of a test plate and incubated at 37° C. in a 5% CO₂ atmosphere. After completion of the incubation for 4 hours, 3 μl of Bio-Glo Luciferase assay reagent (Promega, #G7941, prepared as recommended by the supplier) were added to all wells. The test plate was incubated at 20° C. for 10 min before measurement of the luminescence in a microplate reader (typically Pherastar by BMG, Germany, or ViewLux by Perkin-Elmer, USA). Data were normalized (neutral control=0% effect, high control=100% effect). Compounds were tested in duplicates at up to 11 concentrations (typically 20 μM, 5.7 μM, 1.6 μM, 0.47 μM, 0.13 μM, 38 nM, 11 nM, 3.1 nM, 0.89 nM, 0.25 nM and 0.073 nM). Dilution series were made prior to the assay in a 100-fold concentrated form by serial dilution. EC₅₀ values were calculated by 4-Parameter fitting using a commercial software package (Genedata Analyzer, Switzerland).

### Polyclonal Activation of Human PBMCs

To test the effect of DGKα compounds on IL-2 and IFN-γ secretion of human Peripheral Blood Mononuclear Cells (PBMCs) a 24 h human PBMC assay is performed as screening assay. For this, a 96 well flat bottom plate is coated with a suboptimal stimulation condition (EC 10-30) of human aCD3 (Invitrogen, clone OKT3) antibody in 50 μl PBS/well at 4° C. overnight. PBMCs isolated and frozen at liquid N₂ from leucapherese samples is thawed and resuspended in culture medium (X-Vivo-20). 4×10⁵ cells/well are plated. Wells are treated with the respective compound concentrations (5-fold dilution steps from 10 μM to 3 nM) and the final DMSO concentration per well is 0.1%. Medium+DMSO (0.1%) is used as baseline value. As positive controls 1000 ng/ml aCD3+aCD28 (1 μg/ml) and a DGKα reference compound is used. After 24 h the medium is collected and hIL-2 or hIFN-γ ELISA are performed. The following parameters are calculated: EC₅₀ value, concentration at 50% increase; max increase in % and respective concentration and maximum effect normalized to max concentration (10 μM) of a selected DGKα reference compound.

### In Vitro Activation of Mouse OT-I Antigen-Specific T-Cells

To test the effect of DGKα compounds in murine antigen-specific T-cells, spleens and lymph nodes of OT-I mice are collected and mashed through a 40 μm cell strainer and incubated for 1 min in 1 ml ACK lysing buffer (Gibco)/spleen. 4×106 cells/ml are incubated in medium containing 0.05 ng/ml SIINFEKL in a 50 ml falcon at 37° C. for 30 min. Afterwards cells are centrifuged and 4×106 cells/ml are resuspended in fresh medium (DMEM; 10% FCS, 1% Pen/Strep, 0.1% β-mercaptoethanol, 1% HEPES). 4×105 cells are plated per well in a 96-well round bottom plate. Wells are treated with respective compound concentrations (5-fold dilution steps from 10 μM to 3 nM) in a final DMSO concentration of 0.1%. Medium+DMSO (0.1%) is used as baseline value. As positive controls cells incubated with the 4× SIINFEKL concentration (0.2 ng/ml) and a DGKα reference compound are used. The plates are centrifuged to reduce the distance between T-cells and APCs before incubation. After 24 h the medium is collected and mIL-2 or mIFN-γ ELISAs are performed. The following parameters are calculated: EC₅₀ value, concentration at 50% increase; max increase in % and respective concentration and maximum effect normalized to max concentration (10 μM) of a selected DGKα reference compound.

### DGKα Surface Plasmon Resonance Interaction Assay

The ability of the compounds described in this invention to bind to DGKα may be determined using surface plasmon resonance (SPR). This allows for the quantification of binding in terms of the equilibrium dissociation constant (K_{D} [M]), as well as association and dissociation rate constants (kₒₙ[1/Ms] and k_{off} [1/s], respectively). The measurements may be performed using Biacore® T200, Biacore® S200 or Biacore® 8K (GE Healthcare).

All buffers described in this section were prepared with 10×HBS-P+Buffer (GE Healthcare, #BR100671) supplemented with additional buffer components as indicated below, dithiothreitol (DTT from Sigma, #D0632-25G), Adenosine 5′-triphosphate (ATP from Sigma, #A26209-10G), MgCl₂ (Sigma, #M1028-100ML), dimethyl sulfoxide (DMSO from Biomol, #54686.500).

For SPR measurements, recombinant and biotinylated human DGKα (DGKα_hu_1 Avi) was immobilized via the streptavidin-biotin interaction onto a Series S Sensor Chip SA (GE Healthcare, #BR-1005-31). Briefly, DGKα was diluted to a concentration of 19 μg/ml in Immobilization Buffer (10 mM HEPES, 150 mM NaCl, 0.05% v/v Surfactant P20, 2 mM MgCl₂, 1 mM DTT, pH 7.4) and captured on the SA Chip surface using a flow rate of 10 μl/min for 500 seconds at a temperature of 10° C. Immobilization levels of approximately 8000-10000 RU were typically achieved. The reference surface consisted of a streptavidin surface without immobilized protein. Compounds were diluted from 10 mM DMSO stock solution into Running Buffer (10 mM HEPES, 150 mM NaCl, 0.05% v/v Surfactant P20, 2 mM MgCl₂, 1 mM DTT, 0.2 mM ATP and 1% v/v DMSO, pH 7.4). For SPR-binding measurements serial dilutions (typically 1:3 dilutions resulting in 8 concentrations up to 2 μM or 20 μM) were injected over immobilized protein. Binding affinity and kinetics were measured at 18° C. and at a flow rate of 100 μl/min.

For regeneration of slowly dissociating compounds an additional regeneration step was included by injection of Regeneration Buffer without ATP (10 mM HEPES, 150 mM NaCl, 0.05% v/v Surfactant P20, 1 mM DTT and 1% v/v DMSO, pH 7.4) for 200 s at a flow rate of 30 μl/min

The double-referenced sensorgrams were fit to a simple reversible Langmuir 1:1 reaction mechanism as implemented in the Biacore® T200, S200 and 8K evaluation software (Biacore T200 Evaluation Software version 2.0, Biacore S200 Evaluation Software version 1.0, Biacore 8K Evaluation Software v 1.1.1.7442, GE Healthcare).

### Expression of DGKα in Insect Cells Using the Baculovirus System

Expression Constructs:

The cDNA encoding the full length sequence of human DGKα (Uniprot P23743) was optimized for expression in eukaryotic cells and synthesized by the GeneArt Technology at Life Technologies.

The DNA sequence encoded the following sequence:

Construct DGKα_hu amino acid M1 to S735

Additionally the expression construct encoded: a Kozak DNA sequence for translation initiation (GCCACC), at the C-terminus a Flag (DYKDDDDK) sequence followed by two stop codons and additionally 5′ and 3′ att-DNA sequences for Gateway Cloning.

The DGKα construct was subcloned using the Gateway Technology into the Destination vector pD-INS. The vector pD-INS is a Baculovirus transfer vector (based on vector pVL1393, Pharmingen) which enables the expression of the DGK-Flag protein. The respective protein was named DNA_hu_1.

Additionally the DNA construct DGKα_hu with C-terminal Flag tag was also subcloned in to the Destination vector pD-INSA. This Baculovirus transfer vector is designed to fuse a His6 tag +Avi tag protein sequence to N-terminus of the DGKα_hu-Flag protein. The complete encoded protein was designated DGKα_hu_1Avi. The Avi-tag sequence enables a site-specific in-vitro biotinylation of the DGKα protein.

Generation of Recombinant Baculovirus

In separate approaches each of the two DGK transfer vectors was co-transfected in Sf9 cells with Baculovirus DNA (Flashbac Gold DNA, Oxford Expression Technologies) using Fugene HD (Roche). After 5 days the supernatant of the transfected cells containing the recombinant Baculovirus encoding the various DGK proteins was used for further infection of Sf9 cells for virus amplification whereby the virus titer was monitored using qPCR.

DGK Expression in Sf9 Cells Using Bioreactor

Sf9 cells cultured (Insect-xpress medium, Lonza, 27° C.) in a Wave-bioreactor with a disposable culture bag were infected at a cell density of 10⁶ cells/mL with one of the recombinant baculovirus stocks at a multiplicity of infection of 1 and incubated for 72. Subsequently the cells were harvested by centrifugation (800×g) and cell pellet frozen at −80° C.

To produce biotinylated DGKα_hu_1Avi the Sf9 cells in the bioreactor were co-infected with the Baculovirus encoding DGKα_hu_1Avi as well as with a Baculovirus encoding the biotinylation enzyme BirA.

Purification of the DGK-Flag Proteins:

Purification of the DGK-Flag proteins was achieved by a two-step chromatography procedure as follows.

The pelleted cells (from 8 L cell culture) were resuspended in Lysis-Buffer (50 mM Tris HCl 7.4; 150 mM NaCl; 10 mM MgCl₂; 1 μM CaCl₂; 1 mM DTT; 0.1% NP-40; 0.1% NP-40; Complete Protease Inhibitor Cocktail-(Roche)) and lysed by a freeze-thaw cycle followed by an incubation on ice for 60 min. The lysate was centrifuged at 63.000×g for 30 min. at 4° C. The soluble supernatant was than incubated with 25 mL anti-Flag M2 Agarose (Sigma) in a plastic flask rotating for 16 h at 4° C. for binding of the DGK-Flag proteins, subsequently rinsed with 10×25 mL Wash-Buffer (50 mM Tris HCl 7.4; 150 mM NaCl; 10 mM MgCl₂; 1 μM CaCl₂; 1 mM DTT) and finally the bound protein was eluted using Elusion-Buffer (Wash-Buffer with 300 μg/mL FLAG-Peptide, incubated 30 min. at 4° C. with 3×15 mL).

The elution fractions from the affinity chromatography were concentrated (using Amicon Ultra 15, Centrifugal Filters, 30 kDa MW cut-off; Millipore #UFC903024) to 10 mL and applied to a size exclusion chromatography column (S200 prep grade 26/60, GE Healthcare) and the resulting monomeric peak fraction was collected, pooled and again concentrated. Wash-buffer was used for size exclusion chromatography and the final concentrated sample. The final protein sample concentration was 5-10 mg/mL and the yield was 1-2 mg final protein per L cell culture.

For DGKα_hu_1Avi a biotinylation level of 100% was demonstrated by mass spectromentry.

In Vivo Activation of Murine Antigen Specific OT1 T Cells

Oral Administration of compounds enhances antigen-specific T cell activation in vivo.

Direct detection of antigen-specific T cell proliferation in vivo is technically challenging, since it requires the presence of T cells specific for a cognate antigen and also a specific measurement procedure for cell proliferation. Both these requirements are fulfilled in the OT-1 transfer model, which utilizes the direct transfer of CD8 T cells transgenic for a T cell receptor recognizing an Ovalbumin-derived peptide as antigen. Before transfer, these cells are labeled with the fluorescent dye CFSE, which is diluted by every cell division and therefore allows detection of cell proliferation. After transfer of the CFSE-labeled T cells, mice are vaccinated with the Ovalbumin antigen OVA-30. Only transferred OT-1 cells are able to recognize the OVA-antigen presented by APC and only these transferred T cells then get activated. Flow cytometric analysis of CFSE-levels in the OT-1 cells can be combined with measurement of multiple activation markers like CD69, CD25 and PD1.

In particular, mice receive 2×10×6 CFSE-labeled OT-1 T cells and are vaccinated one day later by intravenous application of 2.5 μg OVA-30. Mice are then divided into groups which receive vehicle only, compound alone or in combination with other immune modulating agents. Mice are treated for 2 to 20 days and T cell composition (incl. transferred OT-1 cells) of spleen, blood and lymphodes are analysed by FACS.

In Vivo Syngeneic Tumor Models

Animals are assigned to a study at the age of 6-8 weeks. Animal husbandry, feeding and health conditions are according to animal welfare guidelines. Syngeinic tumor cell lines are cultivated with appropriate medium and splitted at least 3 times before inoculation. Female mice are inoculated with appropriate amount of tumor cells in medium or a medium/matrigel mixture s.c, i.v. or i.p depending on the model. After 4-10 days the animals are randomized and therapeutic treatment starts when tumors reach a size of approx. 40-70 mm2.

Tumor size is measured using calipers determining length (a) and width (b). Tumor volume is calculated according to:

v=(a×b\hat{ }2)/2

Significance of monotherapies and combination treatment is calculated versus control group as determined by 2-Way ANOVA analysis.

1. A compound of formula (I): 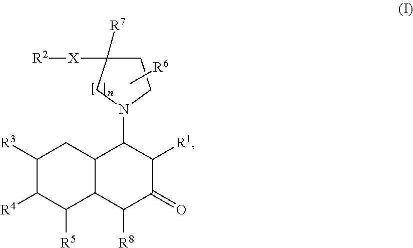
wherein:
n is selected from the group consisting of 1, 2, and 3;
X is selected from the group consisting of —O—, —S—, —S(═O)—, —S(═O)₂—, *-(C(R¹⁶)(R¹⁷))ₒ-#, *—O—C(R¹⁶)₂—(C(R¹⁶)(R¹⁷))ₚ-#, *-(C(R¹⁶)(R¹⁷))ₚ—C(R¹⁶)₂—O—#, 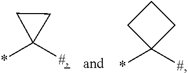
wherein * indicates the point of attachment to R² and #indicates the point of attachment to the pyrrolidine, piperidine, azepane moiety;
R¹ selected from the group consisting of cyano, —C(═O)NH₂, —C(═O)N(H)CH₃, —C(═O)N(H)C₂H₅, —C(═O)N(CH₃)₂, and —C(═O)OR¹⁵;
R² is selected from the group consisting of phenyl, naphthyl, and 5- to 10-membered heteroaryl,
wherein the 5- to 10-membered heteroaryl group is connected to the rest of the molecule via a carbon atom of said 5- to 10-membered heteroaryl group,
wherein the phenyl, naphthyl, and 5- to 10-membered heteroaryl group are optionally substituted, one, two, three, or four times, wherein each substituent independently selected from the group consisting of a halogen atom, C₁-C₆-alkyl, C₃-C₆-cycloalkyl, C₁-C₆-hydroxyalkyl, C₁-C₆-haloalkyl, (C₁-C₂-alkoxy)-(C₁-C₆-alkyl)-, C₁-C₆-alkoxy, (C₁-C₂-alkoxy)-(C₁-C₆-alkoxy)-, C₁-C₆-haloalkoxy, C₃-C₆-cycloalkyloxy, phenoxy, —SR¹⁴, —S(═O)R¹⁴, —S(═O)₂R¹⁴, —P(═O)(R¹⁴)₂, nitro, cyano, hydroxy, —N(R⁹)(R¹⁰), —C(═O)N(R⁹)(R¹⁰), —C(═O)R¹¹, —C(═O)₂R¹⁵, —N(R¹²)C(═O)R¹³, —N(R¹²)S(═O)₂R¹⁴, —N═S(═NH)(R¹⁴)₂, —N═S(═O)(R¹⁴)₂, —S(═O)(═NR¹⁴)R¹³,
4- to 7-membered heterocycloalkyl, 5- to 7-membered heterocycloalkenyl,
(4- to 7-membered heterocycloalkyl)oxy, phenyl, and 5- or 6-membered heteroaryl,
or two substituents of said phenyl group, when they are attached to adjacent ring atoms, are linked to one another in such a way that they jointly form a group selected from the group consisting of —(CH₂)₃—, —(CH₂)₄—, —O—(CH₂)₂—, —(CH₂)₂—O—, —CH₂—O—CH₂—, —O—(CH₂)₃—, —(CH₂)₃—O—, —CH₂—O—(CH₂)₂—, —(CH₂)₂O—CH₂—, —O—CH₂—O—, and —O—(CH₂)₂—O—,
wherein said 4- to 7-membered heterocycloalkyl group and 5- to 7-membered heterocycloalkenyl group are connected to the rest of the molecule via a carbon atom of said 4- to 7-membered heterocycloalkyl group or 5- to 7-membered heterocycloalkenyl group,
wherein said 4- to 7-membered heterocycloalkyl group, 5- to 7-membered heterocycloalkenyl group, and (4- to 7-membered heterocycloalkyl)oxy group are optionally substituted, one, two, or three times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, —N(R⁹)(R¹⁰), and oxo,
wherein said C₁-C₆-alkyl and C₁-C₆-alkoxy groups are optionally substituted with a group selected from the group consisting of C₃-C₄-cycloalkyl, phenyl, and 4- to 7-membered heterocycloalkyl,
wherein said 4- to 7-membered heterocycloalkyl group is connected to the rest of the molecule via a carbon atom of said 4- to 7-membered heterocycloalkyl group,
wherein said 4- to 7-membered heterocycloalkyl group is optionally substituted, one, two, or three times, each substituent independently selected from the group consisting of a halogen atom, C₁-C₂-alkyl, C₁-C₂-haloalkyl,
cyano, hydroxy, C₁-C₂-alkoxy, C₃-C₄-cycloalkyl, —N(R⁹)(R¹⁰), and oxo,
wherein the phenyl group is optionally substituted, one or two times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, and —N(R⁹)(R¹⁰),
which C₃-C₄-cycloalkyl group is optionally substituted, one or two times, wherein each substituent is independently selected from the group consisting of a halogen atom, cyano, and hydroxy,
wherein said C₃-C₆-cycloalkyl group is optionally substituted, one or two times, wherein each substituent is independently a halogen atom or a C₁-C₄-alkyl group,
wherein said phenyl, phenoxy, and 5- or 6-membered heteroaryl group are optionally substituted, one or two times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, and —N(R⁹)(R¹⁰);
R³ is selected from the group consisting of a hydrogen atom, a halogen atom, C₁-C₆-alkyl, C₂-C₆-alkenyl, C₂-C₆-alkynyl, C₃-C₆-cycloalkyl, C₄-C₆-cycloalkenyl, C₁-C₆-hydroxyalkyl, C₁-C₆-haloalkyl, (C₁-C₂-alkoxy)-(C₁-C₆-alkyl)-, C₁-C₆-alkoxy, (C₁-C₂-alkoxy)-(C₁-C₆-alkoxy)-, C₁-C₄-haloalkoxy, C₃-C₆-cycloalkyloxy, phenoxy, —SR¹⁴, —S(═O)R¹⁴, —S(═O)₂R¹⁴, cyano, hydroxy, —N(R⁹)(R¹⁰), —C(═O)N(R⁹)(R¹⁰), —C(═O)R¹¹, —N(R¹²)C(═O)R¹³, —N(R¹²)S(═O)₂R¹⁴, —N═S(═NH)(R¹⁴)₂, —N═S(═O)(R¹⁴)₂ and —P(═O)(R¹⁴)₂,
4- to 7-membered heterocycloalkyl, 5- to 7-membered heterocycloalkenyl,
(4- to 7-membered heterocycloalkyl)oxy, phenyl, and 5- or 6-membered heteroaryl,
wherein said 4- to 7-membered heterocycloalkyl group and 5- to 7-membered heterocycloalkenyl group are connected to the rest of the molecule via a carbon atom of said 4- to 7-membered heterocycloalkyl group or 5- to 7-membered heterocycloalkenyl group,
wherein said 4- to 7-membered heterocycloalkyl group, 5- to 7-membered heterocycloalkenyl group, and (4- to 7-membered heterocycloalkyl)oxy group are optionally substituted, one, two, or three times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, —N(R⁹)(R¹⁰), and oxo,
wherein said C₁-C₆-alkyl, C₂-C₆-alkenyl, C₂-C₆-alkynyl, and C₁-C₆-alkoxy group is optionally substituted with a group selected from C₃-C₄-cycloalkyl, phenyl, and 4- to 7-membered heterocycloalkyl,
wherein said 4- to 7-membered heterocycloalkyl group is connected to the rest of the molecule via a carbon atom of said 4- to 7-membered heterocycloalkyl group,
wherein said 4- to 7-membered heterocycloalkyl group is optionally substituted, one, two, or three times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, —N(R⁹)(R¹⁰), and oxo,
wherein the phenyl group is optionally substituted, one or two times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, and —N(R⁹)(R¹⁰),
wherein the C₃-C₄-cycloalkyl group is optionally substituted, one or two times, wherein each substituent is independently selected from the group consisting of a halogen atom, cyano, and hydroxy,
wherein said C₃-C₆-cycloalkyl and C₄-C₆-cycloalkenyl groups are optionally substituted, one or two times, wherein each substituent is independently a halogen atom or a C₁-C₄-alkyl group,
wherein said phenyl, phenoxy, and 5- or 6-membered heteroaryl group is optionally substituted, one or two times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, and —N(R⁹)(R¹⁰);
R⁴ is selected from the group consisting of a hydrogen atom, a halogen atom, C₁-C₆-alkyl, C₂-C₆-alkenyl, C₂-C₆-alkynyl, C₃-C₆-cycloalkyl, C₄-C₆-cycloalkenyl, C₁-C₆-hydroxyalkyl, C₁-C₆-haloalkyl, (C₁-C₂-alkoxy)-(C₁-C₆-alkyl)-, C₁-C₆-alkoxy, (C₁-C₂-alkoxy)-(C₁-C₆-alkoxy)-, C₁-C₄-haloalkoxy, C₃-C₆-cycloalkyloxy, —S(═O)R¹⁴, —S(═O)₂R¹⁴, cyano, hydroxy, —N(R⁹)(R¹⁰), —N(R¹⁸)(R¹⁹), —C(═O)N(R⁹)(R¹⁰), —C(═O)R¹¹, —N(R¹²)C(═O)R¹³, —N(R¹²)S(═O)₂R¹⁴, —N═S(═NH)(R¹⁴)₂, —N═S(═O)(R¹⁴)₂, —P(═O)(R¹⁴)₂, 4- to 7-membered heterocycloalkyl, 5- to 7-membered heterocycloalkenyl, (4- to 7-membered heterocycloalkyl)oxy, phenyl, and 5- or 6-membered heteroaryl,
wherein said 4- to 7-membered heterocycloalkyl group and 5- to 7-membered heterocycloalkenyl group is connected to the rest of the molecule via a carbon atom of said 4- to 7-membered heterocycloalkyl group or 5- to 7-membered heterocycloalkenyl group,
wherein said 4- to 7-membered heterocycloalkyl group, 5- to 7-membered heterocycloalkenyl group, and (4- to 7-membered heterocycloalkyl)oxy group is optionally substituted, one, two, or three times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, —N(R⁹)(R¹⁰), and oxo,
wherein said C₁-C₆-alkyl, C₂-C₆-alkenyl, C₂-C₆-alkynyl, and C₁-C₆-alkoxy groups are optionally substituted with a group selected from the group consisting of C₃-C₄-cycloalkyl, phenyl, and 4- to 7-membered heterocycloalkyl,
wherein said 4- to 7-membered heterocycloalkyl group is connected to the rest of the molecule via a carbon atom of said 4- to 7-membered heterocycloalkyl group,
wherein said 4- to 7-membered heterocycloalkyl group is optionally substituted, one, two, or three times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, —N(R⁹)(R¹⁰), and oxo,
wherein the phenyl group is optionally substituted, one or two times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, and —N(R⁹)(R¹⁰),
wherein the C₃-C₄-cycloalkyl group is optionally substituted, one or two times, wherein each substituent is independently selected from the group consisting of a halogen atom, cyano, and hydroxy,
wherein said C₃-C₆-cycloalkyl and C₄-C₆-cycloalkenyl groups are optionally substituted, one or two times, wherein each substituent is independently selected from a halogen atom or a C₁-C₄-alkyl group,
wherein said phenyl and 5- or 6-membered heteroaryl groups are optionally substituted, one or two times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, and —N(R⁹)(R¹⁰);
R⁵ is selected from the group consisting of a hydrogen atom, a halogen atom, C₁-C₆-alkyl, C₂-C₆-alkenyl, C₂-C₆-alkynyl, C₃-C₆-cycloalkyl, C₄-C₆-cycloalkenyl, C₁-C₆-hydroxyalkyl, C₁-C₆-haloalkyl, (C₁-C₂-alkoxy)-(C₁-C₆-alkyl)-, C₁-C₆-alkoxy, (C₁-C₂-alkoxy)-(C₁-C₆-alkoxy), —C₁-C₄-haloalkoxy, C₃-C₆-cycloalkyloxy, phenoxy, —SR¹⁴, —S(═O)R¹⁴, S(═O)₂R¹⁴, cyano, hydroxy, —N(R⁹)(R¹⁰), —C(═O)N(R⁹)(R¹⁰), —C(═O)R¹¹, —N(R¹²)C(═O)R¹³, —N(R¹²)S(═O)₂R¹⁴, —N═S(═NH)(R¹⁴)₂, —N═S(═O)(R¹⁴)₂, —P(═O)(R¹⁴)₂, 4- to 7-membered heterocycloalkyl, 5- to 7-membered heterocycloalkenyl, (4- to 7-membered heterocycloalkyl)oxy, phenyl, and 5- or 6-membered heteroaryl,
wherein said 4- to 7-membered heterocycloalkyl group and 5- to 7-membered heterocycloalkenyl group is connected to the rest of the molecule via a carbon atom of said 4- to 7-membered heterocycloalkyl group or 5- to 7-membered heterocycloalkenyl group,
wherein said 4- to 7-membered heterocycloalkyl group, 5- to 7-membered heterocycloalkenyl group, and (4- to 7-membered heterocycloalkyl)oxy group are optionally substituted, one, two, or three times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, —N(R⁹)(R¹⁰), and oxo,
wherein said C₁-C₆-alkyl, C₂-C₆-alkenyl, C₂-C₆-alkynyl, and C₁-C₆-alkoxy group is optionally substituted with a group selected from the group consisting of C₃-C₄-cycloalkyl, phenyl, and 4- to 7-membered heterocycloalkyl,
wherein said 4- to 7-membered heterocycloalkyl group is connected to the rest of the molecule via a carbon atom of said 4- to 7-membered heterocycloalkyl group,
wherein said 4- to 7-membered heterocycloalkyl group is optionally substituted, one, two, or three times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, —N(R⁹)(R¹⁰), and oxo,
wherein the phenyl group is optionally substituted, one or two times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, and —N(R⁹)(R¹⁰),
wherein the C₃-C₄-cycloalkyl group is optionally substituted, one or two times, wherein each substituent is independently selected from the group consisting of a halogen atom, cyano, and hydroxy,
wherein said C₃-C₆-cycloalkyl and C₄-C₆-cycloalkenyl group is optionally substituted, one or two times, wherein each substituent is independently a halogen atom or a C₁-C₄-alkyl group,
wherein said phenyl, phenoxy, and 5- or 6-membered heteroaryl group are optionally substituted, one or two times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, and —N(R⁹)(R¹⁰);
R⁶ is selected from the group consisting of a hydrogen atom, a fluorine atom, C₁-C₄-alkyl, C₁-C₄-hydroxyalkyl, C₁-C₄-alkoxy, hydroxy, and oxo;
R⁷ is selected from the group consisting of a hydrogen atom, a halogen atom, C₁-C₄-alkyl, C₁-C₄-alkoxy, hydroxy, and cyano,
R⁸ is methyl or ethyl;
R⁹ and R¹⁰ are each independently selected from the group consisting of a hydrogen atom, C₁-C₄-alkyl, (C₁-C₄-alkoxy)-(C₂-C₄-alkyl)-, C₃-C₄-cycloalkyl, and C₂-C₄-haloalkyl, or
R⁹ and R¹⁰ together with the nitrogen to which they are attached form a nitrogen containing 4- to 7-membered heterocycloalkyl group or 5- to 7-membered heterocycloalkenyl group,
wherein said nitrogen containing 4- to 7-membered heterocycloalkyl group and 5- to 7-membered heterocycloalkenyl group are optionally substituted, one, two, or three times, wherein each substituent is independently selected from the group consisting of a halogen atom, C₁-C₄-alkyl, C₃-C₄-cycloalkyl, hydroxy, and oxo,
or
two substituents, which are attached to the same carbon atom of said nitrogen containing 4- to 7-membered heterocycloalkyl group, together with the carbon atom to which they are attached, represent form a 4- to 7-membered heterocycloalkyl group,
wherein said 4- to 7-membered heterocycloalkyl group is optionally substituted, one or two times, wherein each substituent is independently selected from the group consisting of a halogen atom, C₁-C₄-alkyl, C₃-C₄-cycloalkyl, C₁-C₄-haloalkyl, hydroxy, and oxo;
R¹¹ is selected from the group consisting of a hydrogen atom, C₁-C₄-alkyl, C₁-C₄-hydroxyalkyl, C₁-C₄-haloalkyl, phenyl, and 5- or 6-membered heteroaryl,
wherein said phenyl group and 5- or 6-membered heteroaryl group is optionally substituted, one or two times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, and —N(R⁹)(R¹⁰);
R¹² is a hydrogen atom or a C₁-C₄-alkyl group;
R¹³ is selected from the group consisting of a hydrogen atom C₁-C₆-alkyl, phenyl, and 5- or 6-membered heteroaryl,
wherein said phenyl group and 5- or 6-membered heteroaryl group are optionally substituted, one or two times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, and —N(R⁹)(R¹⁰);
R¹⁴ is selected from the group consisting of C₁-C₆-alkyl, C₁-C₆-haloalkyl, C₃-C₆-cycloalkyl, phenyl, and 5- or 6-membered heteroaryl,
wherein said phenyl group and 5- or 6-membered heteroaryl group is optionally substituted, one or two times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, and —N(R⁹)(R¹⁰);
R¹⁵ is a hydrogen atom or a C₁-C₄-alkyl group;
each R¹⁶ is independently selected from the group consisting of a hydrogen atom, a halogen atom, C₁-C₂-alkyl, and C₁-C₂-haloalkyl;
each R¹⁷ is independently selected from the group consisting of a hydrogen atom, a halogen atom, C₁-C₂-alkyl, C₁-C₂-haloalkyl, C₁-C₂-alkoxy, C₁-C₂-haloalkoxy, and hydroxy;
R¹⁸ is selected from the group consisting of a hydrogen atom, C₁-C₄-alkyl, C₃-C₄-cycloalkyl, and C₂-C₄-haloalkyl;
R¹⁹ is a 4- to 7-membered heterocycloalkyl group,
wherein said 4- to 7-membered heterocycloalkyl group is optionally substituted, one, two, or three times, wherein each substituent is independently selected from the group consisting of a halogen atom, C₁-C₄-alkyl, C₃-C₄-cycloalkyl, C₁-C₄-alkoxy, hydroxy, and oxo,
and
wherein said 4- to 7-membered heterocycloalkyl group is connected to the rest of the molecule via a carbon atom of the 4- to 7-membered heterocycloalkyl group;
o is 1 or 2; and
p is 0 or 1,
or a stereoisomer, a tautomer, an N-oxide, a hydrate, a solvate, or a salt thereof, or a mixture of any of the foregoing.

2. The compound according to claim 1, wherein:
n is selected from the group consisting of 1, 2, and 3;
X is selected from the group consisting of —O—, —S—, —S(═O)—, —S(═O)₂—, *-(C(R¹⁶)(R¹⁷))ₒ-#, *—O—C(R¹⁶)₂—(C(R¹⁶)(R¹⁷))ₚ-#, *-(C(R¹⁶)(R¹⁷))ₚ—C(R¹⁶)₂—O—#, 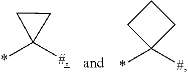
wherein * indicates the point of attachment to R² and #indicates the point of attachment to the pyrrolidine, piperidine, or azepane moiety;
R¹ is selected from the group consisting of cyano, —C(═O)NH₂, —C(═O)N(H)CH₃, —C(═O)N(H)C₂H₅, —C(═O)N(CH₃)₂, and —C(═O)OR¹⁵;
R² is selected from the group consisting of phenyl, naphthyl, and 5- to 10-membered heteroaryl,
wherein the 5- to 10-membered heteroaryl group is connected to the rest of the molecule via a carbon atom of said 5- to 10-membered heteroaryl group,
wherein the phenyl, naphthyl, and 5- to 10-membered heteroaryl group is optionally substituted, one, two, three, or four times, wherein each substituent is independently selected from the group consisting of a halogen atom, C₁-C₄-alkyl, C₃-C₅-cycloalkyl, C₁-C₄-hydroxyalkyl, C₁-C₄-haloalkyl, (C₁-C₂-alkoxy)-(C₁-C₄-alkyl)-, C₁-C₄-alkoxy, (C₁-C₂-alkoxy)-(C₁-C₄-alkoxy)-, C₁-C₄-haloalkoxy, C₃-C₅-cycloalkyloxy, phenoxy, —SR¹⁴, —S(═O)R¹⁴, —S(═O)₂R¹⁴, —P(═O)(R¹⁴)₂, nitro, cyano, hydroxy, —N(R⁹)(R¹⁰), —C(═O)N(R⁹)(R¹⁰), —C(═O)R¹¹, —C(═O)₂R¹⁵, —N(R¹²)C(═O)R¹³, —N(R¹²)S(═O)₂R¹⁴, —N═S(═NH)(R¹⁴)₂, —N═S(═O)(R¹⁴)₂, —S(═O)(═NR¹⁴)R¹³,
4- to 7-membered heterocycloalkyl, 5- to 7-membered heterocycloalkenyl,
(4- to 7-membered heterocycloalkyl)oxy, phenyl and 5- or 6-membered heteroaryl,
or two substituents of said phenyl group, when they are attached to adjacent ring atoms, are linked to one another in such a way that they jointly form a group selected from —(CH₂)₃—, —(CH₂)₄—, —O—(CH₂)₂—, —(CH₂)₂—O—, —CH₂—O—CH₂—, —O—(CH₂)₃—, —(CH₂)₃—O—, —CH₂—O—(CH₂)₂—, —(CH₂)₂O—CH₂—, —O—CH₂—O—, and —O—(CH₂)₂—O—,
wherein said 4- to 7-membered heterocycloalkyl group and 5- to 7-membered heterocycloalkenyl group is connected to the rest of the molecule via a carbon atom of said 4- to 7-membered heterocycloalkyl group or 5- to 7-membered heterocycloalkenyl group,
wherein said 4- to 7-membered heterocycloalkyl group, 5- to 7-membered heterocycloalkenyl group, and (4- to 7-membered heterocycloalkyl)oxy group is optionally substituted, one, two, or three times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, —N(R⁹)(R¹⁰), and oxo,
wherein said C₁-C₄-alkyl and C₁-C₄-alkoxy groups are optionally substituted with a group selected from the group consisting of C₃-C₄-cycloalkyl, phenyl, and 4- to 7-membered heterocycloalkyl,
wherein said 4- to 7-membered heterocycloalkyl group is connected to the rest of the molecule via a carbon atom of said 4- to 7-membered heterocycloalkyl group,
wherein said 4- to 7-membered heterocycloalkyl group is optionally substituted, one, two, or three times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, —N(R⁹)(R¹⁰), and oxo,
wherein the phenyl group is optionally substituted, one or two times, wherein each substituent independently is selected from a halogen atom, C₁-C₂-alkyl, C₁-C₂-haloalkyl, cyano, hydroxy, C₁-C₂-alkoxy, C₃-C₄-cycloalkyl, and —N(R⁹)(R¹⁰),
wherein the C₃-C₄-cycloalkyl group is optionally substituted, one or two times, wherein each substituent is independently selected from the group consisting of a halogen atom, cyano, and hydroxy,
wherein said C₃-C₅-cycloalkyl group is optionally substituted, one or two times, wherein each substituent is independently a halogen atom or a C₁-C₄-alkyl group,
wherein said phenyl, phenoxy, and 5- or 6-membered heteroaryl group is optionally substituted, one or two times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, and —N(R⁹)(R¹⁰);
R³ is selected from the group consisting of a hydrogen atom, a halogen atom, C₁-C₄-alkyl, C₂-C₄-alkenyl, C₂-C₄-alkynyl, C₃-C₅-cycloalkyl, C₄-C₅-cycloalkenyl, C₁-C₄-hydroxyalkyl, C₁-C₄-haloalkyl, (C₁-C₂-alkoxy)-(C₁-C₄-alkyl)-, C₁-C₄-alkoxy, (C₁-C₂-alkoxy)-(C₁-C₄-alkoxy)-, C₁-C₄-haloalkoxy, C₃-C₅-cycloalkyloxy, phenoxy, —SR¹⁴, —S(═O)R¹⁴, —S(═O)₂R¹⁴, cyano, hydroxy, —N(R⁹)(R¹⁰), —C(═O)N(R⁹)(R¹⁰), —C(═O)R¹¹, —N(R¹²)C(═O)R¹³, —N(R¹²)S(═O)₂R¹⁴, —N═S(═NH)(R¹⁴)₂, —N═S(═O)(R¹⁴)₂, —P(═O)(R¹⁴)₂, 4- to 7-membered heterocycloalkyl, 5- to 7-membered heterocycloalkenyl, (4- to 7-membered heterocycloalkyl)oxy, phenyl, and 5- or 6-membered heteroaryl,
wherein said 4- to 7-membered heterocycloalkyl group and 5- to 7-membered heterocycloalkenyl group are connected to the rest of the molecule via a carbon atom of said 4- to 7-membered heterocycloalkyl group or 5- to 7-membered heterocycloalkenyl group,
wherein said 4- to 7-membered heterocycloalkyl group, 5- to 7-membered heterocycloalkenyl group, and (4- to 7-membered heterocycloalkyl)oxy group are optionally substituted, one, two, or three times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, —N(R⁹)(R¹⁰)z and oxo,
wherein said C₁-C₄-alkyl, C₂-C₄-alkenyl, C₂-C₄-alkynyl, and C₁-C₄-alkoxy groups are optionally substituted with a group selected from the group consisting of C₃-C₄-cycloalkyl, phenyl, and 4- to 7-membered heterocycloalkyl,
wherein said 4- to 7-membered heterocycloalkyl group is connected to the rest of the molecule via a carbon atom of said 4- to 7-membered heterocycloalkyl group,
wherein said 4- to 7-membered heterocycloalkyl group is optionally substituted, one, two, or three times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, —N(R⁹)(R¹⁰), and oxo,
wherein the phenyl group is optionally substituted, one or two times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, and —N(R⁹)(R¹⁰),
wherein the C₃-C₄-cycloalkyl group is optionally substituted, one or two times, wherein each substituent is independently selected from the group consisting of a halogen atom, cyano, and hydroxy,
wherein said C₃-C₅-cycloalkyl and C₄-C₅-cycloalkenyl groups are optionally substituted, one or two times, wherein each substituent is independently a halogen atom or a C₁-C₄-alkyl group,
wherein said phenyl, phenoxy, and 5- or 6-membered heteroaryl group is optionally substituted, one or two times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, and —N(R⁹)(R¹⁰);
R⁴ is selected from the group consisting of a hydrogen atom, a halogen atom, C₁-C₄-alkyl, C₂-C₄-alkenyl, C₂-C₄-alkynyl, C₃-C₅-cycloalkyl, C₄-C₅-cycloalkenyl, C₁-C₄-hydroxyalkyl, C₁-C₄-haloalkyl, (C₁-C₂-alkoxy)-(C₁-C₄-alkyl)-, C₁-C₄-alkoxy, (C₁-C₂-alkoxy)-(C₁-C₄-alkoxy)-, C₁-C₄-haloalkoxy, C₃-C₅-cycloalkyloxy, —S(═O)R¹⁴, —S(═O)₂R¹⁴, cyano, hydroxy, —N(R⁹)(R¹⁰), —N(R¹⁸)(R¹⁹), —C(═O)N(R⁹)(R¹⁰), —C(═O)R¹¹, —N(R¹²)C(═O)R¹³, —N(R¹²)S(═O)₂R¹⁴, —N═S(═NH)(R¹⁴)₂, —N═S(═O)(R¹⁴)₂, —P(═O)(R¹⁴)₂, 4- to 7-membered heterocycloalkyl, 5- to 7-membered heterocycloalkenyl, (4- to 7-membered heterocycloalkyl)oxy, phenyl, and 5- or 6-membered heteroaryl,
wherein said 4- to 7-membered heterocycloalkyl group and 5- to 7-membered heterocycloalkenyl group is connected to the rest of the molecule via a carbon atom of said 4- to 7-membered heterocycloalkyl group or 5- to 7-membered heterocycloalkenyl group,
wherein said 4- to 7-membered heterocycloalkyl group, 5- to 7-membered heterocycloalkenyl group, and (4- to 7-membered heterocycloalkyl)oxy group is optionally substituted, one, two, or three times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, —N(R⁹)(R¹⁰), and oxo,
wherein said C₁-C₄-alkyl, C₂-C₄-alkenyl, C₂-C₄-alkynyl, and C₁-C₄-alkoxy groups are optionally substituted with a group selected from C₃-C₄-cycloalkyl, phenyl, and 4- to 7-membered heterocycloalkyl,
wherein said 4- to 7-membered heterocycloalkyl group is connected to the rest of the molecule via a carbon atom of said 4- to 7-membered heterocycloalkyl group,
wherein said 4- to 7-membered heterocycloalkyl group is optionally substituted, one, two, or three times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, —N(R⁹)(R¹⁰), and oxo,
wherein the phenyl group is optionally substituted, one or two times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, and —N(R⁹)(R¹⁰),
wherein the C₃-C₄-cycloalkyl group is optionally substituted, one or two times, wherein each substituent is independently selected from the group consisting of a halogen atom, cyano, and hydroxy,
wherein said C₃-C₅-cycloalkyl and C₄-C₅-cycloalkenyl groups are optionally substituted, one or two times, wherein each substituent is independently a halogen atom or a C₁-C₄-alkyl group,
wherein said phenyl and 5- or 6-membered heteroaryl groups are optionally substituted, one or two times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, and —N(R⁹)(R¹⁰);
R⁵ is selected from the group consisting of a hydrogen atom, a halogen atom, C₁-C₄-alkyl, C₂-C₄-alkenyl, C₂-C₄-alkynyl, C₃-C₅-cycloalkyl, C₄-C₅-cycloalkenyl, C₁-C₄-hydroxyalkyl, C₁-C₄-haloalkyl, (C₁-C₂-alkoxy)-(C₁-C₄-alkyl)-, C₁-C₄-alkoxy, (C₁-C₂-alkoxy)-(C₁-C₄-alkoxy), —C₁-C₄-haloalkoxy, C₃-C₅-cycloalkyloxy, phenoxy, —SR¹⁴, —S(═O)R¹⁴, S(═O)₂R¹⁴, cyano, hydroxy, —N(R⁹)(R¹⁰), —C(═O)N(R⁹)(R¹⁰), —C(═O)R¹¹, —N(R¹²)C(═O)R¹³, —N(R¹²)S(═O)₂R¹⁴, —N═S(═NH)(R¹⁴)₂, —N═S(═O)(R¹⁴)₂, —P(═O)(R¹⁴)₂, 4- to 7-membered heterocycloalkyl, 5- to 7-membered heterocycloalkenyl, (4- to 7-membered heterocycloalkyl)oxy, phenyl, and 5- or 6-membered heteroaryl,
wherein said 4- to 7-membered heterocycloalkyl group and 5- to 7-membered heterocycloalkenyl group are connected to the rest of the molecule via a carbon atom of said 4- to 7-membered heterocycloalkyl group or 5- to 7-membered heterocycloalkenyl group,
wherein said 4- to 7-membered heterocycloalkyl group, 5- to 7-membered heterocycloalkenyl group, and (4- to 7-membered heterocycloalkyl)oxy group are optionally substituted, one, two, or three times, wherein each substituent is independently selected from the group consisting a halogen atom, C₁-C₂-alkyl, C₁-C₂-haloalkyl, cyano, hydroxy, C₁-C₂-alkoxy, C₃-C₄-cycloalkyl, —N(R⁹)(R¹⁰), and oxo,
wherein said C₁-C₄-alkyl, C₂-C₄-alkenyl, C₂-C₄-alkynyl, and C₁-C₄-alkoxy group is optionally substituted with a group selected from the group consisting of C₃-C₄-cycloalkyl, phenyl, and 4- to 7-membered heterocycloalkyl,
wherein said 4- to 7-membered heterocycloalkyl group is connected to the rest of the molecule via a carbon atom of said 4- to 7-membered heterocycloalkyl group,
wherein said 4- to 7-membered heterocycloalkyl group is optionally substituted, one, two, or three times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, —N(R⁹)(R¹⁰), and oxo,
wherein the phenyl group is optionally substituted, one or two times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, and —N(R⁹)(R¹⁰),
wherein the C₃-C₄-cycloalkyl group is optionally substituted, one or two times, wherein each substituent is independently selected from the group consisting of a halogen atom, cyano, and hydroxy,
wherein said C₃-C₅-cycloalkyl and C₄-C₅-cycloalkenyl groups are optionally substituted, one or two times, wherein each substituent is independently a halogen atom or a C₁-C₄-alkyl group,
wherein said phenyl, phenoxy, and 5- or 6-membered heteroaryl group is optionally substituted, one or two times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, and —N(R⁹)(R¹⁰);
R⁶ is selected from the group consisting of a hydrogen atom, a fluorine atom, C₁-C₄-alkyl, C₁-C₄-hydroxyalkyl, C₁-C₄-alkoxy, hydroxy, and oxo;
R⁷ is selected from the group consisting of a hydrogen atom, a halogen atom, C₁-C₄-alkyl, C₁-C₄-alkoxy, hydroxy, and cyano;
R⁸ is methyl or ethyl;
R⁹ and R¹⁰ is selected from the group consisting of a hydrogen atom, C₁-C₄-alkyl, (C₁-C₄-alkoxy)-(C₂-C₄-alkyl)-, C₃-C₄-cycloalkyl, and C₂-C₄-haloalkyl, or
R⁹ and R¹⁰ together with the nitrogen to which they are attached form a nitrogen containing 4- to 7-membered heterocycloalkyl group or 5- to 7-membered heterocycloalkenyl group,
wherein said nitrogen containing 4- to 7-membered heterocycloalkyl group and 5- to 7-membered heterocycloalkenyl group are optionally substituted, one, two, or three times, wherein each substituent is independently selected from the group consisting of a halogen atom, C₁-C₄-alkyl, C₃-C₄-cycloalkyl, hydroxy, and oxo,
or
two substituents, which are attached to the same carbon atom of said nitrogen containing 4- to 7-membered heterocycloalkyl group, together with the carbon atom to which they are attached, form a 4- to 7-membered heterocycloalkyl group,
wherein said 4- to 7-membered heterocycloalkyl group is optionally substituted, one or two times, wherein each substituent is independently selected from the group consisting of a halogen atom, C₁-C₄-alkyl, C₃-C₄-cycloalkyl, C₁-C₄-haloalkyl, hydroxy, and oxo;
R¹¹ is selected from the group consisting of a hydrogen atom, C₁-C₄-alkyl, C₁-C₄-hydroxyalkyl, C₁-C₄-haloalkyl, phenyl, and 5- or 6-membered heteroaryl,
wherein said phenyl group and 5- or 6-membered heteroaryl groups are optionally substituted, one or two times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, and —N(R⁹)(R¹⁰);
R¹² is a hydrogen atom or a C₁-C₄-alkyl group,
R¹³ is selected from the group consisting of a hydrogen atom, C₁-C₄-alkyl, phenyl, and 5- or 6-membered heteroaryl,
wherein said phenyl group and 5- or 6-membered heteroaryl group is optionally substituted, one or two times, each substituent independently selected from a halogen atom or a group selected from
C₁-C₂-alkyl, C₁-C₂-haloalkyl, cyano, hydroxy, C₁-C₂-alkoxy, C₃-C₄-cycloalkyl and
—N(R⁹)(R¹⁰),
R¹⁴ represents a group selected from C₁-C₄-alkyl, C₁-C₄-haloalkyl, C₃-C₅-cycloalkyl, phenyl and 5- or 6-membered heteroaryl,
wherein said phenyl group and 5- or 6-membered heteroaryl groups are optionally substituted, one or two times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, and —N(R⁹)(R¹⁰);
R¹⁵ is a hydrogen atom or a C₁-C₄-alkyl group;
each R¹⁶ is independently selected from the group consisting of a hydrogen atom, a halogen atom, C₁-C₂-alkyl, and C₁-C₂-haloalkyl;
each R¹⁷ is independently selected from the group consisting of a hydrogen atom, a halogen atom, C₁-C₂-alkyl, C₁-C₂-haloalkyl, C₁-C₂-alkoxy, C₁-C₂-haloalkoxy, and hydroxy;
R¹⁸ is selected from the group consisting of a hydrogen atom, C₁-C₄-alkyl, C₃-C₄-cycloalkyl, and C₂-C₄-haloalkyl;
R¹⁹ is a 4- to 7-membered heterocycloalkyl group,
wherein said 4- to 7-membered heterocycloalkyl group is optionally substituted, one, two, or three times, wherein each substituent is independently selected from the group consisting of a halogen atom, C₁-C₄-alkyl, C₃-C₄-cycloalkyl, C₁-C₄-alkoxy, hydroxy, and oxo,
and
wherein said 4- to 7-membered heterocycloalkyl group is connected to the rest of the molecule via a carbon atom of the 4- to 7-membered heterocycloalkyl group;
o is 1 or 2, and
p is 0 or 1,
or a stereoisomer, a tautomer, an N-oxide, a hydrate, a solvate, or a salt thereof, or a mixture of any of the foregoing.

3. The compound according to claim 1, wherein:
n is selected from the group consisting of 1, 2, and 3;
X is selected from the group consisting of —O—, —S—, —S(═O)—, —S(═O)₂—, *-(C(R¹⁶)(R¹⁷))ₒ-#, *—O—C(R¹⁶)₂—(C(R¹⁶)(R¹⁷))ₚ-#, *-(C(R¹⁶)(R¹⁷))ₚ—C(R¹⁶)₂—O—#, 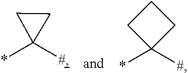
wherein * indicates the point of attachment to R² and #indicates the point of attachment to the pyrrolidine, piperidine, azepane moiety,
R¹ is selected from the group consisting of cyano, —C(═O)NH₂, —C(═O)N(H)CH₃, —C(═O)N(H)C₂H₅, —C(═O)N(CH₃)₂, and —C(═O)OR¹⁵;
R² is selected from the group consisting of phenyl, naphthyl, and 5- to 10-membered heteroaryl,
which 5- to 10-membered heteroaryl group is connected to the rest of the molecule via a carbon atom of said 5- to 10-membered heteroaryl group,
wherein the phenyl, naphthyl, and 5- to 10-membered heteroaryl group are optionally substituted, one, two, three, or four times, wherein each substituent is independently selected from the group consisting of a halogen atom, C₁-C₄-alkyl, C₃-C₅-cycloalkyl, C₁-C₄-haloalkyl, C₁-C₄-alkoxy, (C₁-C₂-alkoxy)-(C₁-C₄-alkoxy)-, C₁-C₄-haloalkoxy, —SR¹⁴, —S(═O)R¹⁴, —S(═O)₂R¹⁴, —P(═O)(R¹⁴)₂, nitro, cyano, hydroxy, —N(R⁹)(R¹⁰), —C(═O)N(R⁹)(R¹⁰), —C(═O)₂R¹⁵, —N(R¹²)S(═O)₂R¹⁴, —N═S(═O)(R¹⁴)₂, —S(═O)(═NR¹⁴)R¹³, 4- to 7-membered heterocycloalkyl, and 5- or 6-membered heteroaryl,
wherein said 4- to 7-membered heterocycloalkyl group is connected to the rest of the molecule via a carbon atom of said 4- to 7-membered heterocycloalkyl group,
wherein said 5- or 6-membered heteroaryl group is optionally substituted, one or two times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, and —N(R⁹)(R¹⁰);
R³ is selected from the group consisting of a hydrogen atom, a halogen atom, C₁-C₄-alkyl, C₃-C₅-cycloalkyl, (C₁-C₂-alkoxy)-(C₁-C₄-alkyl)-, C₁-C₄-alkoxy, —S(═O)₂R¹⁴, cyano, hydroxy, —N(R⁹)(R¹⁰), and —P(═O)(R¹⁴)₂,
R⁴ is selected from the group consisting of a hydrogen atom, a halogen atom, C₁-C₄-alkyl, C₃-C₅-cycloalkyl, (C₁-C₂-alkoxy)-(C₁-C₄-alkyl)-, C₁-C₄-alkoxy, (C₁-C₂-alkoxy)-(C₁-C₄-alkoxy)-, C₃-C₅-cycloalkyloxy, hydroxy, —N(R⁹)(R¹⁰), —N(R¹⁸)(R¹⁹), —P(═O)(R¹⁴)₂, 4- to 7-membered heterocycloalkyl, and (4- to 7-membered heterocycloalkyl)oxy,
wherein said 4- to 7-membered heterocycloalkyl group is connected to the rest of the molecule via a carbon atom of said 4- to 7-membered heterocycloalkyl group,
wherein said 4- to 7-membered heterocycloalkyl group and (4- to 7-membered heterocycloalkyl)oxy group is optionally substituted, one, two, or three times,
wherein each substituent is independently selected from the group consisting of a halogen atom, C₁-C₂-alkyl, C₁-C₂-haloalkyl, cyano,
hydroxy, C₁-C₂-alkoxy, C₃-C₄-cycloalkyl, —N(R⁹)(R¹⁰), and oxo;
R⁵ is selected from the group consisting of a hydrogen atom, a halogen atom, C₁-C₄-alkyl, C₃-C₅-cycloalkyl, C₁-C₄-alkoxy, S(═O)₂R¹⁴, cyano, —N(R⁹)(R¹⁰), —N═S(═O)(R¹⁴)₂, and —P(═O)(R¹⁴)₂;
R⁶ is selected from the group consisting of a hydrogen atom, C₁-C₄-alkyl, C₁-C₄-hydroxyalkyl, C₁-C₄-alkoxy, hydroxy, and oxo;
R⁷ is a hydrogen atom or a C₁-C₄-alkyl group;
R⁸ is methyl or ethyl;
R⁹ and R¹⁰ is a hydrogen atom or C₁-C₄-alkyl group,
or
R⁹ and R¹⁰ together with the nitrogen to which they are attached form a nitrogen containing 4- to 7-membered heterocycloalkyl group or 5- to 7-membered heterocycloalkenyl group,
wherein said nitrogen containing 4- to 7-membered heterocycloalkyl group and 5- to 7-membered heterocycloalkenyl group are optionally substituted, one, two, or three times, wherein each substituent is independently selected from the group consisting of a halogen atom, C₁-C₄-alkyl, C₃-C₄-cycloalkyl, hydroxy, and oxo,
or
two substituents, which are attached to the same carbon atom of said nitrogen containing 4- to 7-membered heterocycloalkyl group, together with the carbon atom to which they are attached, form a 4- to 7-membered heterocycloalkyl group,
wherein said 4- to 7-membered heterocycloalkyl group is optionally substituted, one or two times, wherein each substituent is independently selected from the group consisting of a halogen atom or a group selected from C₁-C₄-alkyl, C₃-C₄-cycloalkyl, C₁-C₄-haloalkyl, hydroxy, and oxo;
R¹² is a hydrogen atom or a C₁-C₄-alkyl group;
R¹³ represents is selected from the group consisting of a hydrogen atom, C₁-C₄-alkyl, phenyl, and 5- or 6-membered heteroaryl,
wherein said phenyl group and 5- or 6-membered heteroaryl group is optionally substituted, one or two times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, and —N(R⁹)(R¹⁰);
R¹⁴ is a C₁-C₄-alkyl group;
R¹⁵ represents is a hydrogen atom or a C₁-C₄-alkyl group;
each R¹⁶ is independently a hydrogen atom or a C₁-C₂-alkyl group;
each R¹⁷ is a hydroxy group;
R¹⁸ is a hydrogen atom or a C₁-C₄-alkyl group;
R¹⁹ is a 4- to 7-membered heterocycloalkyl group,
wherein said 4- to 7-membered heterocycloalkyl group is optionally substituted, one or two times, with a C₁-C₄-alkyl group,
and
wherein said 4- to 7-membered heterocycloalkyl group is connected to the rest of the molecule via a carbon atom of the 4- to 7-membered heterocycloalkyl group;
o is 1 or 2; and
p is 0 or 1,
or a stereoisomer, a tautomer, an N-oxide, a hydrate, a solvate, or a salt thereof, or a mixture of any of the foregoing.

4. The compound according to claim 1, wherein:
n selected from the group consisting of 1, 2, and 3;
X is selected from the group consisting of —O—, —S—, *-(C(R¹⁶)(R¹⁷))ₒ-#, *—O—C(R¹⁶)₂—(C(R¹⁶)(R¹⁷))ₚ-#and *-(C(R¹⁶)(R¹⁷))ₚ—C(R¹⁶)₂—O—#,
wherein * indicates the point of attachment to R² and #indicates the point of attachment to the pyrrolidine, piperidine, azepane moiety;
R¹ is selected from the group consisting of cyano, —C(═O)NH₂, —C(═O)N(H)CH₃, —C(═O)N(CH₃)₂, and —C(═O)OR¹⁵;
R² is selected from the group consisting of phenyl, naphthyl, and 5- to 10-membered heteroaryl,
wherein 5- to 10-membered heteroaryl group is connected to the rest of the molecule via a carbon atom of said 5- to 10-membered heteroaryl group,
wherein phenyl, naphthyl, and 5- to 10-membered heteroaryl group is optionally substituted, one, two, three, or four times, wherein each substituent is independently selected from the group consisting of a halogen atom, C₁-C₄-alkyl, C₃-C₅-cycloalkyl, C₁-C₄-haloalkyl, C₁-C₄-alkoxy, (C₁-C₂-alkoxy)-(C₁-C₄-alkoxy)-, C₁-C₄-haloalkoxy, —SR¹⁴, —S(═O)R¹⁴, —S(═O)₂R¹⁴, nitro, cyano, hydroxy, —N(R⁹)(R¹⁰), —C(═O)N(R⁹)(R¹⁰), —C(═O)₂R¹⁵, —N═S(═O)(R¹⁴)₂_{₂} and 5- or 6-membered heteroaryl,
wherein said 5- or 6-membered heteroaryl group is optionally substituted, one or two times, wherein each substituent is independently selected from the group consisting of a halogen atom, C₁-C₂-alkyl, C₁-C₂-haloalkyl, cyano, hydroxy, C₁-C₂-alkoxy, C₃-C₄-cycloalkyl, and —N(R⁹)(R¹⁰);
R³ is selected from the group consisting of a hydrogen atom, a halogen atom, C₁-C₄-alkyl, C₃-C₅-cycloalkyl, C₁-C₄-alkoxy, cyano, and hydroxy;
R⁴ is a hydrogen atom, a halogen atom, C₁-C₄-alkyl, C₃-C₅-cycloalkyl, (C₁-C₂-alkoxy)-(C₁-C₄-alkyl)-, C₁-C₄-alkoxy, (C₁-C₂-alkoxy)-(C₁-C₄-alkoxy)-, C₃-C₅-cycloalkyloxy, hydroxy, —N(R⁹)(R¹⁰), —N(R¹⁸)(R¹⁹), —P(═O)(R¹⁴)₂, 4- to 7-membered heterocycloalkyl, and (4- to 7-membered heterocycloalkyl)oxy,
wherein said 4- to 7-membered heterocycloalkyl group is connected to the rest of the molecule via a carbon atom of said 4- to 7-membered heterocycloalkyl group;
R⁵ represents is selected from the group consisting of a hydrogen atom, a halogen atom, S(═O)₂R¹⁴, cyano, —N(R⁹)(R¹⁰), —N═S(═O)(R¹⁴)₂, and —P(═O)(R¹⁴)₂;
R⁶ is a hydrogen atom;
R⁷ is a hydrogen atom or a C₁-C₄-alkyl group;
R⁸ is a methyl group;
R⁹ and R¹⁰ is a hydrogen atom or C₁-C₄-alkyl group,
or
R⁹ and R¹⁰ together with the nitrogen to which they are attached form a nitrogen containing 4- to 7-membered heterocycloalkyl group or 5- to 7-membered heterocycloalkenyl group,
wherein said nitrogen containing 4- to 7-membered heterocycloalkyl group and 5- to 7-membered heterocycloalkenyl group are optionally substituted, one, two, or three times, wherein each substituent is independently selected from the group consisting of a halogen atom, C₁-C₄-alkyl, and oxo;
R¹⁴ is a C₁-C₄-alkyl group;
R¹⁵ is C₁-C₄-alkyl group;
R¹⁶ is a hydrogen atom or a methyl group;
R¹⁷ is a hydroxy group;
R¹⁸ is a hydrogen atom or a C₁-C₄-alkyl group;
R¹⁹ is a 4- to 7-membered heterocycloalkyl group,
wherein said 4- to 7-membered heterocycloalkyl group is optionally substituted, one or two times, with a C₁-C₄-alkyl group,
and
wherein said 4- to 7-membered heterocycloalkyl group is connected to the rest of the molecule via a carbon atom of the 4- to 7-membered heterocycloalkyl group;
o is 1; and
p is 0,
or a stereoisomer, a tautomer, an N-oxide, a hydrate, a solvate, or a salt thereof, or a mixture of any of the foregoing.

5. The compound according to claim 1, wherein:
n is selected from the group consisting of 1, 2, and 3;
X is selected from the group consisting of —O—, —S—, *-(C(R¹⁶)(R¹⁷))ₒ-#, *—O—C(R¹⁶)₂—(C(R¹⁶)(R¹⁷))ₚ-#, and *-(C(R¹⁶)(R¹⁷))ₚ—C(R¹⁶)₂—O—#,
wherein * indicates the point of attachment to R² and #indicates the point of attachment to the pyrrolidine, piperidine, azepane moiety;
R¹ is selected from the group consisting of cyano, —C(═O)NH₂, —C(═O)N(H)CH₃, —C(═O)N(CH₃)₂, and —C(═O)OR¹⁵;
R² is selected from the group consisting of phenyl, naphthyl, and 5- to 10-membered heteroaryl,
wherein 5- to 10-membered heteroaryl group is selected from the group consisting of pyridinyl, pyrimidinyl, 1,2-benzoxazolyl, 1,3-benzoxazolyl, and 1,3-benzothiazolyl,
wherein phenyl, naphthyl, and 5- to 10-membered heteroaryl group is optionally substituted, one, two, three, or four times, wherein each substituent is independently selected from the group consisting of a halogen atom, C₁-C₄-alkyl, C₃-C₅-cycloalkyl, C₁-C₄-haloalkyl, C₁-C₄-alkoxy, (C₁-C₂-alkoxy)-(C₁-C₄-alkoxy)-, C₁-C₄-haloalkoxy, —SR¹⁴, —S(═O)R¹⁴, —S(═O)₂R¹⁴, nitro, cyano, hydroxy, —N(R⁹)(R¹⁰), —C(═O)N(R⁹)(R¹⁰), —C(═O)₂R¹⁵, —N═S(═O)(R¹⁴)₂_{₂} and 5- or 6-membered heteroaryl,
wherein said 5- or 6-membered heteroaryl group is optionally substituted, one or two times, wherein each substituent is independently a halogen atom or a C₁-C₂-alkyl group;
R³ is selected from the group consisting of a hydrogen atom, a halogen atom, C₁-C₄-alkyl, C₃-C₅-cycloalkyl, C₁-C₄-alkoxy, cyano, and hydroxy;
R⁴ is selected from the group consisting of a hydrogen atom, a halogen atom, C₁-C₄-alkyl, C₃-C₅-cycloalkyl, (C₁-C₂-alkoxy)-(C₁-C₄-alkyl)-, C₁-C₄-alkoxy, (C₁-C₂-alkoxy)-(C₁-C₄-alkoxy)-, C₃-C₅-cycloalkyloxy, hydroxy, —N(R⁹)(R¹⁰), —N(R¹⁸)(R¹⁹), —P(═O)(R¹⁴)₂, 4- to 7-membered heterocycloalkyl, and (4- to 7-membered heterocycloalkyl)oxy,
wherein said 4- to 7-membered heterocycloalkyl group is connected to the rest of the molecule via a carbon atom of said 4- to 7-membered heterocycloalkyl group;
R⁵ is selected from the group consisting of a hydrogen atom, a halogen atom, S(═O)₂R¹⁴, cyano, —N(R⁹)(R¹⁰), —N═S(═O)(R¹⁴)₂, and —P(═O)(R¹⁴)₂;
R⁶ is a hydrogen atom;
R⁷ is a hydrogen atom or C₁-C₄-alkyl group;
R⁸ represents a methyl group,
R⁹ and R¹⁰ are each independently a hydrogen atom or C₁-C₄-alkyl group,
or
R⁹ and R¹⁰ together with the nitrogen to which they are attached form a nitrogen containing 4- to 7-membered heterocycloalkyl group or 5- to 7-membered heterocycloalkenyl group,
wherein said nitrogen containing 4- to 7-membered heterocycloalkyl group and 5- to 7-membered heterocycloalkenyl group are optionally substituted, one, two, or three times, wherein each substituent is independently selected from the group consisting of a halogen atom, C₁-C₄-alkyl, and oxo;
R¹⁴ is a C₁-C₄-alkyl group;
R¹⁵ C₁-C₄-alkyl group;
each R¹⁶ is independently a hydrogen atom or a methyl group;
R¹⁷ is a hydroxy group;
R¹⁸ is a hydrogen atom or a C₁-C₄-alkyl group;
R¹⁹ is a 4- to 7-membered heterocycloalkyl group,
wherein said 4- to 7-membered heterocycloalkyl group is connected to the rest of the molecule via a carbon atom of the 4- to 7-membered heterocycloalkyl group;
o is 1; and
p is 0,
or a stereoisomer, a tautomer, an N-oxide, a hydrate, a solvate, or a salt thereof, or a mixture of any of the foregoing.

6. The compound according to claim 1, wherein:
n is selected from the group consisting of 1, 2, and 3;
X is selected from the group consisting of —O—, —S—, —CH₂—, —CH(OH)—, *—OCH₂-#, *-CH₂O—#, and *-CH(CH₃)O—#,
wherein * indicates the point of attachment to R² and #indicates the point of attachment to the pyrrolidine, piperidine, or azepane moiety;
R¹ is cyano or —C(═O)NH₂;
R² is selected from the group consisting of phenyl, pyridin-2-yl, pyridin-3-yl, pyrimidin-5-yl, 1,2-benzoxazol-6-yl, 1,3-benzoxazol-4-yl, and 1,3-benzothiazol-2-yl,
wherein R² is optionally substituted, one or two times, wherein each substituent is independently selected from the group consisting of a fluorine atom, a chlorine atom, a bromine atom, methyl, cyclopropyl, difluoromethyl, trifluoromethyl, methoxy, ethoxy, isopropoxy, 2-methoxyethoxy, difluoromethoxy, trifluormethoxy, methylsulfanyl, methylsulfinyl, methylsulfonyl, nitro, cyano, amino, dimethylamino, azetidin-1-yl, 2-oxopyrrolidin-1-yl, 3-methyl-2-oxo-2,3-dihydro-1H-imidazol-1-yl, 2-oxopiperidin-1-yl, 4-methyl-2-oxopiperazin-1-yl, morpholino-4-yl, 3-methyl-2-oxoimidazolidin-1-yl, 3-methyl-2-oxo-1,3-diazinan-1-yl, carbamoyl, dimethylcarbamoyl, ethoxycarbonyl, [dimethyl(oxido)-λ⁶-sulfanylidene]amino, 1H-imidazol-1-yl, 1-methyl-1H-pyrazol-4-yl, and 1H-1,2,4-triazol-1-yl;
R³ is selected from the group consisting of a hydrogen atom, a fluorine atom, a chlorine atom, a bromine atom, methyl, cyclopropyl, methoxy, cyano, and hydroxy;
R⁴ is selected from the group consisting of a hydrogen atom, a fluorine atom, a chlorine atom, a bromine atom, methyl, cyclopropyl, 2-methoxyethyl, methoxy, propoxy, 2-methoxyethoxy, cyclopropyloxy, hydroxy, 2-oxopyrrolidin-1-yl, 4-methylpiperazin-1-yl, methyl(tetrahydrofuran-3-yl)amino, dimethylphosphoryl, oxetan-3-yl, (oxetan-3-yl)oxy, tetrahydrofuranyl-3-oxy, (tetrahydro-2H-pyran-3-yl)oxy, and (tetrahydro-2H-pyran-4-yl)oxy;
R⁵ is selected from the group consisting of a hydrogen atom, a fluorine atom, a chlorine atom, a bromine atom, methanesulfonyl, cyano, 2-oxopyrrolidin-1-yl, [dimethyl(oxido)-λ⁶-sulfanylidene]amino, and dimethylphosphoryl;
R⁶ is a hydrogen atom;
R⁷ is a hydrogen atom or a methyl group; and
R⁸ is a methyl group,
or a stereoisomer, a tautomer, an N-oxide, a hydrate, a solvate, or a salt thereof, or a mixture of any of the foregoing.

7. The compound according to claim 1, wherein the compound is selected from the group consisting of:
1-methyl-4-[4-(4-methylphenoxy)piperidin-1-yl]-2-oxo-1,2-dihydroquinoline-3-carbonitrile;
1-methyl-2-oxo-4-{4-[4-(trifluoromethoxy)phenoxy]piperidin-1-yl}-1,2-dihydroquinoline-3-carbonitrile;
1-methyl-4-[4-(3-methylphenoxy)piperidin-1-yl]-2-oxo-1,2-dihydroquinoline-3-carbonitrile;
4-[4-(3-methoxyphenoxy)piperidin-1-yl]-1-methyl-2-oxo-1,2-dihydroquinoline-3-carbonitrile;
1-methyl-2-oxo-4-(4-phenoxypiperidin-1-yl)-1,2-dihydroquinoline-3-carbonitrile;
4-[4-(4-fluorophenoxy)piperidin-1-yl]-1-methyl-2-oxo-1,2-dihydroquinoline-3-carbonitrile;
4-[4-(3-fluorophenoxy)piperidin-1-yl]-1-methyl-2-oxo-1,2-dihydroquinoline-3-carbonitrile;
4-[4-(4-bromophenoxy)piperidin-1-yl]-1-methyl-2-oxo-1,2-dihydroquinoline-3-carbonitrile;
4-[4-(3-chlorophenoxy)piperidin-1-yl]-1-methyl-2-oxo-1,2-dihydroquinoline-3-carbonitrile;
4-[4-(4-chlorophenoxy)piperidin-1-yl]-1-methyl-2-oxo-1,2-dihydroquinoline-3-carbonitrile;
4-[4-(2-methoxyphenoxy)piperidin-1-yl]-1-methyl-2-oxo-1,2-dihydroquinoline-3-carbonitrile;
ethyl 4-{[1-(3-cyano-1-methyl-2-oxo-1,2-dihydroquinolin-4-yl)piperidin-4-yl]oxy}benzoate;
1-methyl-2-oxo-4-{4-[4-(trifluoromethyl)phenoxy]piperidin-1-yl}-1,2-dihydroquinoline-3-carbonitrile;
1-methyl-4-[4-(2-methylphenoxy)piperidin-1-yl]-2-oxo-1,2-dihydroquinoline-3-carbonitrile;
1-methyl-2-oxo-4-{4-[3-(trifluoromethyl)phenoxy]piperidin-1-yl}-1,2-dihydroquinoline-3-carbonitrile;
4-[4-(2-chlorophenoxy)piperidin-1-yl]-1-methyl-2-oxo-1,2-dihydroquinoline-3-carbonitrile;
1-methyl-2-oxo-4-{4-[4-(1H-1,2,4-triazol-1-yl)phenoxy]piperidin-1-yl}-1,2-dihydroquinoline-3-carbonitrile;
1-methyl-2-oxo-4-{4-[4-(2-oxopyrrolidin-1-yl)phenoxy]piperidin-1-yl}-1,2-dihydroquinoline-3-carbonitrile;
4-{4-[4-(1H-imidazol-1-yl)phenoxy]piperidin-1-yl}-1-methyl-2-oxo-1,2-dihydroquinoline-3-carbonitrile;
1-methyl-4-{4-[4-(morpholin-4-yl)phenoxy]piperidin-1-yl}-2-oxo-1,2-dihydroquinoline-3-carbonitrile;
1-methyl-4-{4-[4-(3-methyl-2-oxo-1,3-diazinan-1-yl)phenoxy]piperidin-1-yl}-2-oxo-1,2-dihydroquinoline-3-carbonitrile;
1-methyl-4-{4-[4-(3-methyl-2-oxoimidazolidin-1-yl)phenoxy]piperidin-1-yl}-2-oxo-1,2-dihydroquinoline-3-carbonitrile;
1-methyl-2-oxo-4-{4-[4-(2-oxopiperidin-1-yl)phenoxy]piperidin-1-yl}-1,2-dihydroquinoline-3-carbonitrile;
1-methyl-4-{4-[4-(4-methyl-2-oxopiperazin-1-yl)phenoxy]piperidin-1-yl}-2-oxo-1,2-dihydroquinoline-3-carbonitrile;
1-methyl-4-{4-[4-(3-methyl-2-oxo-2,3-dihydro-1H-imidazol-1-yl)phenoxy]piperidin-1-yl}-2-oxo-1,2-dihydroquinoline-3-carbonitrile;
4-{[1-(3-cyano-1-methyl-2-oxo-1,2-dihydroquinolin-4-yl)piperidin-4-yl]oxy}-N,N-dimethylbenzamide;
4-{4-[(1,3-benzoxazol-4-yl)oxy]piperidin-1-yl}-1-methyl-2-oxo-1,2-dihydroquinoline-3-carbonitrile;
1-methyl-2-oxo-4-(4-{[2-(trifluoromethyl)pyrimidin-5-yl]oxy}piperidin-1-yl)-1,2-dihydroquinoline-3-carbonitrile;
4-{4-[(1,2-benzoxazol-6-yl)oxy]piperidin-1-yl}-1-methyl-2-oxo-1,2-dihydroquinoline-3-carbonitrile;
4-[4-(4-{[dimethyl(oxo)-λ⁶-sulfanylidene]amino}phenoxy)piperidin-1-yl]-1-methyl-2-oxo-1,2-dihydroquinoline-3-carbonitrile;
4-[4-(2-methoxy-4-methylphenoxy)piperidin-1-yl]-1-methyl-2-oxo-1,2-dihydroquinoline-3-carbonitrile;
4-[4-(4-cyanophenoxy)piperidin-1-yl]-1-methyl-2-oxo-1,2-dihydroquinoline-3-carbonitrile;
4-[4-(2-fluorophenoxy)piperidin-1-yl]-1-methyl-2-oxo-1,2-dihydroquinoline-3-carbonitrile;
4-[4-(2-cyanophenoxy)piperidin-1-yl]-1-methyl-2-oxo-1,2-dihydroquinoline-3-carbonitrile;
4-{[1-(3-cyano-1-methyl-2-oxo-1,2-dihydroquinolin-4-yl)piperidin-4-yl]oxy}benzamide;
1-methyl-2-oxo-4-{4-[2-(trifluoromethyl)phenoxy]piperidin-1-yl}-1,2-dihydroquinoline-3-carbonitrile;
4-[4-(4-cyclopropylphenoxy)piperidin-1-yl]-1-methyl-2-oxo-1,2-dihydroquinoline-3-carbonitrile;
1-methyl-2-oxo-4-{4-[3-(trifluoromethoxy)phenoxy]piperidin-1-yl}-1,2-dihydroquinoline-3-carbonitrile;
4-{4-[4-(difluoromethoxy)phenoxy]piperidin-1-yl}-1-methyl-2-oxo-1,2-dihydroquinoline-3-carbonitrile;
1-methyl-4-[4-methyl-4-(4-nitrophenoxy)piperidin-1-yl]-2-oxo-1,2-dihydroquinoline-3-carbonitrile;
4-[4-(4-aminophenoxy)-4-methylpiperidin-1-yl]-1-methyl-2-oxo-1,2-dihydroquinoline-3-carbonitrile;
4-[4-(4-bromophenoxy)-4-methylpiperidin-1-yl]-1-methyl-2-oxo-1,2-dihydroquinoline-3-carbonitrile;
4-{4-[4-(methanesulfonyl)phenoxy]piperidin-1-yl}-1-methyl-2-oxo-1,2-dihydroquinoline-3-carbonitrile;
4-{4-[4-(2-methoxyethoxy)phenoxy]piperidin-1-yl}-1-methyl-2-oxo-1,2-dihydroquinoline-3-carbonitrile;
4-{4-[4-(dimethylamino)phenoxy]piperidin-1-yl}-1-methyl-2-oxo-1,2-dihydroquinoline-3-carbonitrile;
4-[4-(4-methoxyphenoxy)piperidin-1-yl]-1-methyl-2-oxo-1,2-dihydroquinoline-3-carbonitrile;
1-methyl-2-oxo-4-(4-{4-[(propan-2-yl)oxy]phenoxy}piperidin-1-yl)-1,2-dihydroquinoline-3-carbonitrile;
1-methyl-2-oxo-4-{4-[4-(trifluoromethoxy)phenoxy]piperidin-1-yl}-1,2-dihydroquinoline-3-carboxamide;
4-[4-(3-chlorophenoxy)piperidin-1-yl]-1-methyl-2-oxo-1,2-dihydroquinoline-3-carboxamide;
4-[4-(4-chlorophenoxy)piperidin-1-yl]-1-methyl-2-oxo-1,2-dihydroquinoline-3-carboxamide;
ethyl 4-{[1-(3-carbamoyl-1-methyl-2-oxo-1,2-dihydroquinolin-4-yl)piperidin-4-yl]oxy}benzoate;
4-{4-[4-(difluoromethoxy)phenoxy]piperidin-1-yl}-1-methyl-2-oxo-1,2-dihydroquinoline-3-carboxamide;
1-methyl-2-oxo-4-[4-(phenylsulfanyl)piperidin-1-yl]-1,2-dihydroquinoline-3-carbonitrile;
7-bromo-1-methyl-2-oxo-4-(4-phenoxypiperidin-1-yl)-1,2-dihydroquinoline-3-carbonitrile;
7-bromo-1-methyl-2-oxo-4-[4-(phenylsulfanyl)piperidin-1-yl]-1,2-dihydroquinoline-3-carbonitrile;
7-bromo-4-[4-(4-fluorophenoxy)piperidin-1-yl]-1-methyl-2-oxo-1,2-dihydroquinoline-3-carbonitrile;
7-bromo-4-[4-(4-chlorophenoxy)piperidin-1-yl]-1-methyl-2-oxo-1,2-dihydroquinoline-3-carbonitrile;
7-bromo-1-methyl-2-oxo-4-{4-[4-(trifluoromethoxy)phenoxy]piperidin-1-yl}-1,2-dihydroquinoline-3-carbonitrile;
7-bromo-1-methyl-2-oxo-4-{4-[4-(trifluoromethyl)phenoxy]piperidin-1-yl}-1,2-dihydroquinoline-3-carbonitrile;
7-bromo-4-[4-(3-chlorophenoxy)piperidin-1-yl]-1-methyl-2-oxo-1,2-dihydroquinoline-3-carbonitrile;
7-bromo-1-methyl-2-oxo-4-{4-[3-(trifluoromethoxy)phenoxy]piperidin-1-yl}-1,2-dihydroquinoline-3-carbonitrile;
7-hydroxy-1-methyl-2-oxo-4-{4-[4-(trifluoromethoxy)phenoxy]piperidin-1-yl}-1,2-dihydroquinoline-3-carbonitrile;
7-fluoro-1-methyl-2-oxo-4-{4-[4-(trifluoromethoxy)phenoxy]piperidin-1-yl}-1,2-dihydroquinoline-3-carbonitrile;
7-chloro-1-methyl-2-oxo-4-{4-[4-(trifluoromethoxy)phenoxy]piperidin-1-yl}-1,2-dihydroquinoline-3-carbonitrile;
4-[4-(4-chlorophenoxy)piperidin-1-yl]-7-methoxy-1-methyl-2-oxo-1,2-dihydroquinoline-3-carbonitrile;
7-methoxy-1-methyl-2-oxo-4-{4-[4-(trifluoromethyl)phenoxy]piperidin-1-yl}-1,2-dihydroquinoline-3-carbonitrile;
1,7-dimethyl-2-oxo-4-{4-[4-(trifluoromethoxy)phenoxy]piperidin-1-yl}-1,2-dihydroquinoline-3-carbonitrile;
7-cyclopropyl-1-methyl-2-oxo-4-[4-(phenylsulfanyl)piperidin-1-yl]-1,2-dihydroquinoline-3-carbonitrile;
7-butoxy-1-methyl-2-oxo-4-[4-(phenylsulfanyl)piperidin-1-yl]-1,2-dihydroquinoline-3-carbonitrile;
7-(cyclopropyloxy)-1-methyl-2-oxo-4-(4-phenoxypiperidin-1-yl)-1,2-dihydroquinoline-3-carbonitrile;
7-methoxy-1-methyl-2-oxo-4-{4-[4-(trifluoromethoxy)phenoxy]piperidin-1-yl}-1,2-dihydroquinoline-3-carbonitrile;
4-[4-(4-chlorophenoxy)piperidin-1-yl]-1-methyl-7-(oxetan-3-yl)-2-oxo-1,2-dihydroquinoline-3-carbonitrile;
4-[4-(3-chlorophenoxy)piperidin-1-yl]-1-methyl-7-(oxetan-3-yl)-2-oxo-1,2-dihydroquinoline-3-carbonitrile;
4-[4-(3-chlorophenoxy)piperidin-1-yl]-7-(2-methoxyethyl)-1-methyl-2-oxo-1,2-dihydroquinoline-3-carbonitrile;
4-[4-(4-chlorophenoxy)piperidin-1-yl]-7-(2-methoxyethyl)-1-methyl-2-oxo-1,2-dihydroquinoline-3-carbonitrile;
7-(2-methoxyethyl)-1-methyl-2-oxo-4-{4-[3-(trifluoromethoxy)phenoxy]piperidin-1-yl}-1,2-dihydroquinoline-3-carbonitrile;
1-methyl-7-(oxetan-3-yl)-2-oxo-4-{4-[3-(trifluoromethoxy)phenoxy]piperidin-1-yl}-1,2-dihydroquinoline-3-carbonitrile;
7-(dimethylphosphoryl)-1-methyl-2-oxo-4-{4-[3-(trifluoromethoxy)phenoxy]piperidin-1-yl}-1,2-dihydroquinoline-3-carbonitrile;
7-(dimethylphosphoryl)-1-methyl-2-oxo-4-{4-[4-(trifluoromethoxy)phenoxy]piperidin-1-yl}-1,2-dihydroquinoline-3-carbonitrile;
4-[4-(3-chlorophenoxy)piperidin-1-yl]-1-methyl-7-(4-methylpiperazin-1-yl)-2-oxo-1,2-dihydroquinoline-3-carbonitrile;
7-(2-methoxyethoxy)-1-methyl-2-oxo-4-{4-[4-(trifluoromethoxy)phenoxy]piperidin-1-yl}-1,2-dihydroquinoline-3-carbonitrile;
1-methyl-2-oxo-7-(2-oxopyrrolidin-1-yl)-4-{4-[4-(trifluoromethoxy)phenoxy]piperidin-1-yl}-1,2-dihydroquinoline-3-carbonitrile;
1-methyl-2-oxo-7-(2-oxopyrrolidin-1-yl)-4-[4-(phenylsulfanyl)piperidin-1-yl]-1,2-dihydroquinoline-3-carbonitrile;
4-[4-(4-fluorophenoxy)piperidin-1-yl]-1-methyl-2-oxo-7-(2-oxopyrrolidin-1-yl)-1,2-dihydroquinoline-3-carbonitrile;
4-[4-(4-chlorophenoxy)piperidin-1-yl]-1-methyl-2-oxo-7-(2-oxopyrrolidin-1-yl)-1,2-dihydroquinoline-3-carbonitrile;
1-methyl-2-oxo-7-(2-oxopyrrolidin-1-yl)-4-{4-[4-(trifluoromethyl)phenoxy]piperidin-1-yl}-1,2-dihydroquinoline-3-carbonitrile;
7-cyclopropyl-1-methyl-2-oxo-4-{4-[4-(trifluoromethyl)phenoxy]piperidin-1-yl}-1,2-dihydroquinoline-3-carbonitrile;
1,7-dimethyl-2-oxo-4-(4-phenoxypiperidin-1-yl)-1,2-dihydroquinoline-3-carbonitrile[[,]];
7-methoxy-1-methyl-2-oxo-4-(4-phenoxypiperidin-1-yl)-1,2-dihydroquinoline-3-carbonitrile;
7-methoxy-1-methyl-2-oxo-4-[4-(phenylsulfanyl)piperidin-1-yl]-1,2-dihydroquinoline-3-carbonitrile;
4-(4-benzylpiperidin-1-yl)-1-methyl-2-oxo-1,2-dihydroquinoline-3-carbonitrile;
4-{4-[(4-fluorophenyl)methyl]piperidin-1-yl}-1-methyl-2-oxo-1,2-dihydroquinoline-3-carbonitrile;
4-{4-[(1,3-benzothiazol-2-yl)methyl]piperidin-1-yl}-1-methyl-2-oxo-1,2-dihydroquinoline-3-carbonitrile;
4-{4-[(2-methoxyphenyl)methyl]piperidin-1-yl}-1-methyl-2-oxo-1,2-dihydroquinoline-3-carbonitrile;
4-{4-[(4-cyanophenyl)methyl]piperidin-1-yl}-1-methyl-2-oxo-1,2-dihydroquinoline-3-carbonitrile;
1-methyl-2-oxo-7-(2-oxopyrrolidin-1-yl)-4-{4-[4-(trifluoromethoxy)phenoxy]piperidin-1-yl}-1,2-dihydroquinoline-3-carboxamide;
4-[4-(4-chlorophenoxy)piperidin-1-yl]-1-methyl-2-oxo-7-(2-oxopyrrolidin-1-yl)-1,2-dihydroquinoline-3-carboxamide;
7-chloro-1-methyl-2-oxo-4-{4-[4-(trifluoromethoxy)phenoxy]piperidin-1-yl}-1,2-dihydroquinoline-3-carboxamide;
7-fluoro-1-methyl-2-oxo-4-{4-[4-(trifluoromethoxy)phenoxy]piperidin-1-yl}-1,2-dihydroquinoline-3-carboxamide;
(rac)-1-methyl-2-oxo-4-{4-[4-(trifluoromethoxy)phenoxy]azepan-1-yl}-1,2-dihydroquinoline-3-carbonitrile;
1-methyl-2-oxo-4-{(4S)-4-[4-(trifluoromethoxy)phenoxy]azepan-1-yl}-1,2-dihydroquinoline-3-carbonitrile;
1-methyl-2-oxo-4-{(4R)-4-[4-(trifluoromethoxy)phenoxy]azepan-1-yl}-1,2-dihydroquinoline-3-carbonitrile;
(rac)-4-[4-(4-bromophenoxy)azepan-1-yl]-1-methyl-2-oxo-1,2-dihydroquinoline-3-carbonitrile;
(rac)-4-{4-[4-(azetidin-1-yl)phenoxy]azepan-1-yl}-1-methyl-2-oxo-1,2-dihydroquinoline-3-carbonitrile;
(rac)-1-methyl-4-{4-[4-(1-methyl-1H-pyrazol-4-yl)phenoxy]azepan-1-yl}-2-oxo-1,2-dihydroquinoline-3-carbonitrile;
4-[4-(benzyloxy)piperidin-1-yl]-1-methyl-2-oxo-1,2-dihydroquinoline-3-carbonitrile;
1-methyl-4-{4-[(4-methylphenyl)methoxy]piperidin-1-yl}-2-oxo-1,2-dihydroquinoline-3-carbonitrile;
1-methyl-4-{4-[(3-methylphenyl)methoxy]piperidin-1-yl}-2-oxo-1,2-dihydroquinoline-3-carbonitrile;
4-{4-[(4-methoxyphenyl)methoxy]piperidin-1-yl}-1-methyl-2-oxo-1,2-dihydroquinoline-3-carbonitrile;
4-{4-[(4-fluorophenyl)methoxy]piperidin-1-yl}-1-methyl-2-oxo-1,2-dihydroquinoline-3-carbonitrile;
4-{4-[(2-fluorophenyl)methoxy]piperidin-1-yl}-1-methyl-2-oxo-1,2-dihydroquinoline-3-carbonitrile;
(rac)-1-methyl-2-oxo-4-{4-[1-phenylethoxy]piperidin-1-yl}-1,2-dihydroquinoline-3-carbonitrile;
4-{4-[(3-fluorophenyl)methoxy]piperidin-1-yl}-1-methyl-2-oxo-1,2-dihydroquinoline-3-carbonitrile;
1-methyl-4-{4-[(2-methylphenyl)methoxy]piperidin-1-yl}-2-oxo-1,2-dihydroquinoline-3-carbonitrile;
4-{4-[(3-methoxyphenyl)methoxy]piperidin-1-yl}-1-methyl-2-oxo-1,2-dihydroquinoline-3-carbonitrile;
4-{4-[(4-chlorophenoxy)methyl]piperidin-1-yl}-1-methyl-2-oxo-1,2-dihydroquinoline-3-carbonitrile;
4-{4-[(2-methoxyphenyl)methoxy]piperidin-1-yl}-1-methyl-2-oxo-1,2-dihydroquinoline-3-carbonitrile;
1-methyl-2-oxo-4-[4-(phenoxymethyl)piperidin-1-yl]-1,2-dihydroquinoline-3-carbonitrile;
4-{4-[(3-chlorophenoxy)methyl]piperidin-1-yl}-1-methyl-2-oxo-1,2-dihydroquinoline-3-carbonitrile;
4-{4-[(2-chlorophenoxy)methyl]piperidin-1-yl}-1-methyl-2-oxo-1,2-dihydroquinoline-3-carbonitrile;
4-[4-(benzyloxy)piperidin-1-yl]-1-methyl-2-oxo-1,2-dihydroquinoline-3-carboxamide;
1-methyl-4-{4-[(4-methylphenyl)methoxy]piperidin-1-yl}-2-oxo-1,2-dihydroquinoline-3-carboxamide;
1-methyl-4-{4-[(3-methylphenyl)methoxy]piperidin-1-yl}-2-oxo-1,2-dihydroquinoline-3-carboxamide;
1-methyl-4-{4-[(2-methylphenyl)methoxy]piperidin-1-yl}-2-oxo-1,2-dihydroquinoline-3-carboxamide;
4-{4-[(3-methoxyphenyl)methoxy]piperidin-1-yl}-1-methyl-2-oxo-1,2-dihydroquinoline-3-carboxamide;
4-{4-[(2-fluorophenyl)methoxy]piperidin-1-yl}-1-methyl-2-oxo-1,2-dihydroquinoline-3-carboxamide;
4-{4-[(4-fluorophenyl)methoxy]piperidin-1-yl}-1-methyl-2-oxo-1,2-dihydroquinoline-3-carboxamide;
4-{4-[(3-fluorophenyl)methoxy]piperidin-1-yl}-1-methyl-2-oxo-1,2-dihydroquinoline-3-carboxamide;
8-fluoro-1-methyl-2-oxo-4-{4-[4-(trifluoromethoxy)phenoxy]piperidin-1-yl}-1,2-dihydroquinoline-3-carbonitrile;
8-bromo-1-methyl-2-oxo-4-{4-[4-(trifluoromethoxy)phenoxy]piperidin-1-yl}-1,2-dihydroquinoline-3-carbonitrile;
8-chloro-1-methyl-2-oxo-4-{4-[4-(trifluoromethoxy)phenoxy]piperidin-1-yl}-1,2-dihydroquinoline-3-carbonitrile;
1-methyl-2-oxo-4-{4-[4-(trifluoromethoxy)phenoxy]piperidin-1-yl}-1,2-dihydroquinoline-3,8-dicarbonitrile;
1-methyl-2-oxo-8-(2-oxopyrrolidin-1-yl)-4-{4-[4-(trifluoromethoxy)phenoxy]piperidin-1-yl}-1,2-dihydroquinoline-3-carbonitrile;
8-{[dimethyl(oxo)-λ⁶-sulfanylidene]amino}-1-methyl-2-oxo-4-{4-[4-(trifluoromethoxy)phenoxy]piperidin-1-yl}-1,2-dihydroquinoline-3-carbonitrile;
8-(dimethylphosphoryl)-1-methyl-2-oxo-4-{4-[4-(trifluoromethoxy)phenoxy]piperidin-1-yl}-1,2-dihydroquinoline-3-carbonitrile;
8-(methanesulfonyl)-1-methyl-2-oxo-4-{4-[4-(trifluoromethoxy)phenoxy]piperidin-1-yl}-1,2-dihydroquinoline-3-carbonitrile;
6-bromo-4-[4-(4-fluorophenoxy)piperidin-1-yl]-1-methyl-2-oxo-1,2-dihydroquinoline-3-carbonitrile;
6-bromo-4-[4-(3-methoxyphenoxy)piperidin-1-yl]-1-methyl-2-oxo-1,2-dihydroquinoline-3-carbonitrile;
6-bromo-1-methyl-2-oxo-4-{4-[4-(trifluoromethoxy)phenoxy]piperidin-1-yl}-1,2-dihydroquinoline-3-carbonitrile;
6-bromo-1-methyl-2-oxo-4-[4-(phenylsulfanyl)piperidin-1-yl]-1,2-dihydroquinoline-3-carbonitrile;
4-[4-(4-fluorophenoxy)piperidin-1-yl]-1,6-dimethyl-2-oxo-1,2-dihydroquinoline-3-carbonitrile;
1,6-dimethyl-2-oxo-4-[4-(phenylsulfanyl)piperidin-1-yl]-1,2-dihydroquinoline-3-carbonitrile;
1,6-dimethyl-2-oxo-4-{4-[4-(trifluoromethoxy)phenoxy]piperidin-1-yl}-1,2-dihydroquinoline-3-carbonitrile;
1-methyl-2-oxo-4-[4-(phenylsulfanyl)piperidin-1-yl]-1,2-dihydroquinoline-3,6-dicarbonitrile;
1-methyl-2-oxo-4-{4-[4-(trifluoromethoxy)phenoxy]piperidin-1-yl}-1,2-dihydroquinoline-3,6-dicarbonitrile;
4-[4-(4-fluorophenoxy)piperidin-1-yl]-1-methyl-2-oxo-1,2-dihydroquinoline-3,6-dicarbonitrile
6-cyclopropyl-1-methyl-2-oxo-4-[4-(phenylsulfanyl)piperidin-1-yl]-1,2-dihydroquinoline-3-carbonitrile;
6-cyclopropyl-4-[4-(4-fluorophenoxy)piperidin-1-yl]-1-methyl-2-oxo-1,2-dihydroquinoline-3-carbonitrile;
1,6-dimethyl-4-[4-(3-methylphenoxy)piperidin-1-yl]-2-oxo-1,2-dihydroquinoline-3-carbonitrile;
1,6-dimethyl-4-[4-(2-methylphenoxy)piperidin-1-yl]-2-oxo-1,2-dihydroquinoline-3-carbonitrile;
4-[4-(2-chlorophenoxy)piperidin-1-yl]-1,6-dimethyl-2-oxo-1,2-dihydroquinoline-3-carbonitrile;
4-[4-(3-methoxyphenoxy)piperidin-1-yl]-1,6-dimethyl-2-oxo-1,2-dihydroquinoline-3-carbonitrile;
4-[4-(2-methoxy-4-methylphenoxy)piperidin-1-yl]-1,6-dimethyl-2-oxo-1,2-dihydroquinoline-3-carbonitrile;
4-[4-(2-methoxyphenoxy)piperidin-1-yl]-1,6-dimethyl-2-oxo-1,2-dihydroquinoline-3-carbonitrile;
4-[4-(2-cyanophenoxy)piperidin-1-yl]-1,6-dimethyl-2-oxo-1,2-dihydroquinoline-3-carbonitrile;
6-methoxy-1-methyl-2-oxo-4-{4-[4-(trifluoromethoxy)phenoxy]piperidin-1-yl}-1,2-dihydroquinoline-3-carbonitrile;
6-chloro-1-methyl-2-oxo-4-{4-[4-(trifluoromethoxy)phenoxy]piperidin-1-yl}-1,2-dihydroquinoline-3-carbonitrile;
1,6-dimethyl-4-[4-(3-methylphenoxy)piperidin-1-yl]-2-oxo-1,2-dihydroquinoline-3-carboxamide;
4-[4-(3-methoxyphenoxy)piperidin-1-yl]-1,6-dimethyl-2-oxo-1,2-dihydroquinoline-3-carboxamide;
1,6-dimethyl-2-oxo-4-{4-[4-(trifluoromethoxy)phenoxy]piperidin-1-yl}-1,2-dihydroquinoline-3-carboxamide;
4-[4-(2-methoxy-4-methylphenoxy)piperidin-1-yl]-1,6-dimethyl-2-oxo-1,2-dihydroquinoline-3-carboxamide;
4-[4-(2-methoxyphenoxy)piperidin-1-yl]-1,6-dimethyl-2-oxo-1,2-dihydroquinoline-3-carboxamide;
4-[4-(2-chlorophenoxy)piperidin-1-yl]-1,6-dimethyl-2-oxo-1,2-dihydroquinoline-3-carboxamide;
8-chloro-1-methyl-2-oxo-4-{4-[4-(trifluoromethoxy)phenoxy]piperidin-1-yl}-1,2-dihydroquinoline-3-carboxamide;
6-fluoro-1-methyl-2-oxo-4-{4-[4-(trifluoromethoxy)phenoxy]piperidin-1-yl}-1,2-dihydroquinoline-3-carboxamide;
6-fluoro-1-methyl-2-oxo-4-{4-[4-(trifluoromethoxy)phenoxy]piperidin-1-yl}-1,2-dihydroquinoline-3-carbonitrile;
6-hydroxy-1-methyl-2-oxo-4-{4-[4-(trifluoromethoxy)phenoxy]piperidin-1-yl}-1,2-dihydroquinoline-3-carbonitrile;
8-bromo-1,6-dimethyl-2-oxo-4-{4-[4-(trifluoromethoxy)phenoxy]piperidin-1-yl}-1,2-dihydroquinoline-3-carbonitrile; and
8-bromo-4-[4-(3-chlorophenoxy)piperidin-1-yl]-1,6-dimethyl-2-oxo-1,2-dihydroquinoline-3-carbonitrile,
or a stereoisomer, a tautomer, an N-oxide, a hydrate, a solvate, or a salt thereof, or a mixture of any of the foregoing.

8. A method for treatment or prophylaxis of a disease, comprising administering a therapeutically effective amount of the compound of claim 1, or a pharmaceutically acceptable salt thereof, to a mammal in need thereof.

9. A pharmaceutical composition comprising the compound of claim 1, or a pharmaceutically acceptable salt thereof, and one or more pharmaceutically acceptable excipients.

10. A pharmaceutical combination comprising:
one or more compounds of claim 1, or a pharmaceutically acceptable salt thereof, and
one or more further active ingredients.

11-14. (canceled)

15. The method of claim 8, wherein the disease is cancer, a condition with dysregulated immune response, or a disorder associated with aberrant DGKα signalling.

16. The pharmaceutical combination of claim 10, wherein the one or more further active ingredients comprises an immune checkpoint inhibitor.

17. The pharmaceutical combination of claim 10, wherein the immune checkpoint inhibitor is an aPD-1/L-1 axis antagonist.

18. The pharmaceutical combination of claim 10, wherein the immune checkpoint inhibitor is an inhibitor of DKGζ.
